(19) United States
(12) Patent Application Publication
(10) Pub. No.: US 2021/0275657 A1
(43) Pub. Date: Sep. 9, 2021

(54) NEOANTIGENS AND USES THEREOF

(71) Applicant: BioNTech US Inc., Cambridge, MA (US)

(72) Inventors: Vikram Juneja, Waltham, MA (US); Zhengxin Dong, Holliston, MA (US); Robyn Jessica Eisert, Somerville, MA (US)

(21) Appl. No.: 17/253,922

(22) PCT Filed: Jun. 19, 2019
(86) PCT No.: PCT/US2019/038061

§ 371 Date: Dec. 18, 2020

Related U.S. Application Data
(60) Provisional application No. 62/687,191, filed on Jun. 19, 2018.
(60) Provisional application No. 62/702,567, filed on Jul. 24, 2018.
(60) Provisional application No. 62/726,804, filed on Sep. 4, 2018.
(60) Provisional application No. 62/789,162, filed on Jan. 7, 2019.
(60) Provisional application No. 62/800,700, filed on Feb. 4, 2019.
(60) Provisional application No. 62/800,792, filed on Feb. 4, 2019.
(60) Provisional application No. 62/801,981, filed on Feb. 6, 2019.

(51) Int. Cl. A61K 39/00; A61K 39/39; C07K 14/47; A61K 31/4196; A61K 31/565; A61K 31/519; A61K 31/506; A61K 31/635; A61K 31/661; A61K 31/4025; A61K 31/473; A61K 31/52; A61K 31/4745; A61K 31/5377; A61K 31/453; A61K 31/498; A61K 31/343; A61K 31/585; A61K 31/58; A61P 35/00

(52) U.S. Cl. CPC A61K 39/001152; A61K 2039/55561; C07K 14/4748; A61K 31/4196; A61K 31/565; A61K 31/519; A61K 31/506; A61K 31/635; A61K 31/661; A61K 31/4025; A61K 31/473; A61K 31/52; A61K 31/4745; A61K 31/5377; A61K 31/453; A61K 31/498; A61K 31/343; A61K 31/585; A61K 31/58; A61P 35/00; A61K 39/39

(57) ABSTRACT

The disclosure herein relates to immunotherapeutic compositions comprising immunotherapeutic peptides comprising neoepitopes. Also disclosed herein are polynucleotides encoding the immunotherapeutic peptides. Also disclosed herein are methods of synthesis of immunotherapeutic peptides comprising neoepitopes and use of the immunotherapeutic compositions including methods of treatment.

### CROSS-REFERENCE

This application claims the benefit of U.S. Provisional Application No. 62/687,191, filed Jun. 19, 2018, U.S. Provisional Application No. 62/702,567, filed Jul. 24, 2018, U.S. Provisional Application No. 62/726,804, filed Sep. 4, 2018, U.S. Provisional Application No. 62/789,162, filed Jan. 7, 2019, U.S. Provisional Application No. 62/801,981, filed Feb. 6, 2019, U.S. Provisional Application No. 62/800,700, filed Feb. 4, 2019, and U.S. Provisional Application No. 62/800,792, filed Feb. 4, 2019, each of which application is incorporated herein by reference in their entirety.

### BACKGROUND

Cancer immunotherapy is the use of the immune system to treat cancer. Immunotherapies exploit the fact that cancer cells often have molecules on their surface that can be detected by the immune system, known as tumor antigens, which are often proteins or other macromolecules (e.g. carbohydrates). Active immunotherapy directs the immune system to attack tumor cells by targeting tumor antigens. Passive immunotherapies enhance existing anti-tumor responses and include the use of monoclonal antibodies, lymphocytes and cytokines. Tumor vaccines are typically composed of tumor antigens and immunostimulatory molecules (e.g., adjuvants, cytokines or TLR ligands) that work together to induce antigen-specific cytotoxic T cells (CTLs) that recognize and lyse tumor cells. One of the critical barriers to developing curative and tumor-specific immunotherapy is the identification and selection of highly specific and restricted tumor antigens to avoid autoimmunity.

Tumor neoantigens, which arise as a result of genetic change (e.g., inversions, translocations, deletions, missense mutations, splice site mutations, etc.) within malignant cells, represent the most tumor-specific class of antigens and can be patient-specific or shared. Tumor neoantigens are unique to the tumor cell as the mutation and its corresponding protein are present only in the tumor. They also avoid central tolerance and are therefore more likely to be immunogenic. Therefore, tumor neoantigens provide an excellent target for immune recognition including by both humoral and cellular immunity. However, tumor neoantigens have rarely been used in cancer vaccine or immunogenic compositions due to technical difficulties in identifying them, selecting optimized antigens, and producing neoantigens for use in a vaccine or immunogenic composition. Accordingly, there is still a need for developing additional cancer therapeutics.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In one aspect provided herein is a pharmaceutical composition comprising (a) at least one polypeptide or a pharmaceutically acceptable salt thereof comprising a first mutant GATA3 peptide sequence and a second mutant GATA3 peptide sequence, wherein (i) the first mutant GATA3 peptide sequence and the second mutant GATA3 peptide sequence each comprise at least 8 contiguous amino acids of SEQ ID NO: 1, and (ii) a C-terminal sequence of the first mutant GATA3 peptide sequence overlaps with an N-terminal sequence of the second mutant GATA3 peptide sequence; wherein the at least 8 contiguous amino acids of SEQ ID NO: 1 comprises at least one amino acid of sequence: PGRPLQTHVLPEPHLALQPLQPHADHAHADAPAIQPVLWTTPPLQHGHRHGLEPCSMLTGPPARVPA VPFDLHFCRSSIMKPKRDGYMFLKAESKIMFATLQRSSLWCLCSNH (SEQ ID NO: 2), or (b) at least one polynucleotide comprising a sequence encoding the at least one polypeptide.

In some embodiments, the first mutant GATA3 peptide sequence or the second mutant GATA3 peptide sequence comprises at least 8 contiguous amino acids of SEQ ID NO: 2. In some embodiments, the first mutant GATA3 peptide sequence and the second mutant peptide sequence comprises at least 8 contiguous amino acids of SEQ ID NO: 2.

In some embodiments, the at least 8 contiguous amino acids of SEQ ID NO: 2 comprises at least 8 contiguous amino acids of sequence:

(SEQ ID NO: 3)
PGRPLQTHVLPEPHLALQPLQPHADHAHADAPAIQPVLWTTPPLQHGHRH
GL.

In some embodiments, the at least 8 contiguous amino acids of SEQ ID NO: 2 comprises at least one amino acid of sequence:

(SEQ ID NO: 4)
EPCSMLTGPPARVPAVPFDLHFCRSSIMKPKRDGYMFLKAESKIMFATLQ
RSSLWCLCSNH.

In some embodiments, at least one of the first mutant GATA3 peptide sequence and the second mutant GATA3 peptide sequence comprise at least 14 mutant amino acids. In some embodiments, the at least one polypeptide comprises at least 3 mutant GATA3 peptide sequences. In some embodiments, the at least one polypeptide comprises at least two polypeptides. In some embodiments, the at least one polypeptide further comprises a third mutant GATA3 peptide sequence, wherein the third mutant GATA3 peptide sequence comprises at least 8 contiguous amino acids of SEQ ID NO: 1, wherein the at least 8 contiguous amino acids of SEQ ID NO: 1 comprises at least one amino acid of sequence SEQ ID NO: 2. In some embodiments, the third GATA3 mutant peptide comprises at least 8 contiguous amino acids of SEQ ID NO: 2.

In some embodiments, the at least one polypeptide comprises at least one mutant GATA3 peptide sequence that binds to or is predicted to bind to a protein encoded by an HLA-A02:01 allele, an HLA-A24:02 allele, an HLA-A03:01 allele, an HLA-B07:02 allele and/or an HLA-B08:01 allele. In some embodiments, the at least one polypeptide comprises at least one mutant GATA3 peptide sequence that binds to or is predicted to bind to a protein encoded by: (a) an HLA-A02:01 allele and an HLA-A24:02 allele, (b) an HLA-A02:01 allele and an HLA-B08:01 allele, (c) an HLA-A24:02 allele and an HLA-B08:01 allele, or (d) HLA-A02:01 allele, an HLA-A24:02 allele and an HLA-B08:01 allele. In some embodiments, (a) the first mutant GATA3 peptide sequence binds to or is predicted to bind to a protein encoded by an HLA-A02:01 allele, an HLA-A24:02 allele, an HLA-A03:01 allele, an HLA-B07:02 allele or an HLA-B08:01 allele; and (b) the second GATA3 peptide sequence binds to or is predicted to bind to a protein encoded by an HLA-A02:01 allele, an HLA-A24:02 allele, an HLA-A03:01 allele, an HLA-B07:02 allele or an HLA-B08:01 allele; wherein the first mutant GATA3 peptide sequence binds to or is predicted to bind to a protein encoded by different HLA allele than the second mutant GATA3 peptide sequence.

In some embodiments, at least one of the first mutant GATA3 peptide sequence and the second mutant GATA 3 peptide sequence binds to a protein encoded by an HLA allele with an affinity of less than 500 nM.

In some embodiments, at least one of the first mutant GATA3 peptide sequence and the second mutant peptide sequence binds to a protein encoded by an HLA allele with a stability of greater than 1 hour.

In some embodiments, the at least one polypeptide comprises at least one of the following sequences: (a) TLQRSSLWCL, VLPEPHLAL, HVLPEPHLAL, ALQPLQPHA, AIQPVLWTT, APAIQPVLWTT, SMLTGPPARV, MLTGPPARV, and/or YMFLKAESKI, and/or (b) MFLKAESKI and/or YMFLKAESKI, and/or (c) VLWTTPPLQH, YMFLKAESK and/or KIMFATLQR, and/or (d) FATLQRSSL, EPHLALQPL, QPVLWTTPPL, GPPARVPAV, MFATLQRSSL KPKRDGYMF and/or KPKRDGYMFL, and/or (e) IMKPKRDGYM, MFATLQRSSL, FLKAESKIMF, LHFCRSSIM, EPHLALQPL, FATLQRSSL, ESKIMFATL, FLKAESKIM and/or YMFLKAESKI.

In some embodiments, the at least one polypeptide comprises at least two of the following sequences: (a) TLQRSSLWCL, VLPEPHLAL, HVLPEPHLAL, ALQPLQPHA, AIQPVLWTT, APAIQPVLWTT, SMLTGPPARV, MLTGPPARV, and/or YMFLKAESKI, and/or (b) MFLKAESKI and/or YMFLKAESKI, and/or (c) VLWTTPPLQH, YMFLKAESK and/or KIMFATLQR, and/or (d) FATLQRSSL, EPHLALQPL, QPVLWTTPPL, GPPARVPAV, MFATLQRSSL KPKRDGYMF and/or KPKRDGYMFL, and/or (e) IMKPKRDGYM, MFATLQRSSL, FLKAESKIMF, LHFCRSSIM EPHLALQPL, FATLQRSSL, ESKIMFATL, FLKAESKIM and/or YMFLKAESKI.

In some embodiments, the mutant GATA3 peptide sequences comprise, (a) the first mutant GATA3 peptide sequence from (a) and the second mutant GATA3 peptide sequence from (b), (b) the first mutant GATA3 peptide sequence from (a) and the second mutant GATA3 peptide sequence from (c), (c) the first mutant GATA3 peptide sequence from (a) and the second mutant GATA3 peptide sequence from (d), (d) the first mutant GATA3 peptide sequence from (a) and the second mutant GATA3 peptide sequence from (e), (e) the first mutant GATA3 peptide sequence from (b) and the second mutant GATA3 peptide sequence from (c), (f) the first mutant GATA3 peptide sequence from (b) and the second mutant GATA3 peptide sequence from (d), (g) the first mutant GATA3 peptide sequence from (b) and the second mutant GATA3 peptide sequence from (e), (h) the first mutant GATA3 peptide sequence from (c) and the second mutant GATA3 peptide sequence from (d), (i) the first mutant GATA3 peptide sequence from (c) and the second mutant GATA3 peptide sequence from (e), or (j) the first mutant GATA3 peptide sequence from (d) and the second mutant GATA3 peptide sequence from (e).

In some embodiments, the first mutant GATA3 peptide sequences, and the second mutant GATA 3 peptide sequence comprises a peptide of Table 5 and/or Table 6. In some embodiments, the first mutant GATA3 peptide sequence comprises a first neoepitope of GATA3 protein and the second peptide mutant GATA3 peptide sequence comprises a second neoepitope of a mutant GATA protein, wherein the first mutant GATA3 peptide sequence is different from the second mutant GATA3 peptide sequence, and wherein the first neoepitope comprises at least one mutant amino acid and the second neoepitope comprises the same mutant amino acid.

In some embodiments, each of the first mutant GATA3 peptide sequence and the second mutant GATA3 peptide sequences comprising the at least eight contiguous amino acids are represented by a formula of: [Xaa]F-[Xaa]N-[Xaa]C or [Xaa]N-[Xaa]C-[Xaa]F, wherein each Xaa is an amino acid, wherein [Xaa]N and [Xaa]C each comprise an amino acid sequence encoded by a different portion of the GATA3 gene, wherein [Xaa]F is any amino acid sequence, wherein [Xaa]N is encoded in a non-wild type reading frame of the GATA3 gene, wherein [Xaa]C comprises the at least one mutant amino acid and is encoded in a non-wild type reading frame of the GATA3 gene, wherein N is an integer of from 0-100, wherein C is an integer of from 1-100, wherein F is an integer of from 0-100, wherein the sum of N and M is at least 8.

In some embodiments, each Xaa of [Xaa]F is a lysine residue and F is an integer of from 1-100, 1-10, 9, 8, 7, 6, 5, 4, 3, 2 or 1. In some embodiments, F is 3, 4 or 5.

In some embodiments, each of the mutant GATA3 peptide sequences are present at a concentration of at least 50 μg/mL-400 μg/mL. In some embodiments, the first mutant GATA3 peptide sequences and the second mutant GATA3 peptide sequence comprises a sequence of Table 1 or 2. In some embodiments, the composition further comprises an immunomodulatory agent or an adjuvant. In some embodiments, the adjuvant is polyICLC.

In one aspect, provided herein is a pharmaceutical composition comprising: one or more mutant GATA3 peptide sequence, the one or more mutant GATA3 peptide sequence comprises a sequence selected from group consisting of ESKIMFATLQRSSL, KPKRDGYMFLKAESKI, SMLTGPPARVPAVPFDLH, EPCSMLTGPPARVPAVPFDLH, LHFCRSSIMKPKRDGYMFLKAESKI, GPPARVPAVPFDLHFCRSSIMKPKRD, and KPKRDGYMFLKAESKIMFATLQRSSLWCLCSNH.

In some embodiments, the one or more mutant GATA3 peptide sequence is ESKIMFATLQRSSL. In some embodiments, the one or more mutant GATA3 peptide sequence is KPKRDGYMFLKAESKI. In some embodiments, the one or more mutant GATA3 peptide sequence is SMLTGPPARVPAVPFDLH. In some embodiments, the one or more mutant GATA3 peptide sequence is EPCSMLTGPPARVPAVPFDLH. In some embodiments, the one or more mutant GATA3 peptide sequence is LHFCRSSIMKPKRDGYMFLKAESKI. In some embodiments, the one or more mutant GATA3 peptide sequence is GPPARVPAVPFDLHFCRSSIMKPKRD. In some embodiments, the one or more mutant GATA3 peptide sequence is KPKRDGYMFLKAESKIMFATLQRSSLWCLCSNH.

In some embodiments, the pharmaceutical composition comprises a pH modifier present at a concentration of from 0.1 mM-1 mM. In some embodiments, the pharmaceutical composition comprises a pH modifier present at a concentration of from 1 mM-10 mM.

In one aspect provided herein is a method of synthesizing a GATA3 peptide, wherein the peptide comprises a sequence of at least two contiguous amino acids selected from the group consisting of Xaa-Cys, Xaa-Ser, and Xaa-Thr, wherein Xaa is any amino acid, the method comprising: (a) coupling at least one di-peptide or derivative thereof to an amino acid or derivative thereof of a GATA3 peptide or derivative thereof to obtain a pseudo-proline containing GATA3 peptide or derivative thereof, wherein the di-peptide or derivative thereof comprises a pseudo-proline moiety, (b) coupling one or more selected amino acids, small peptides or derivatives thereof to the pseudo-proline containing GATA3 peptide or derivative thereof, and (c) cleaving the pseudo-proline containing GATA3 peptide or derivative thereof from the resin. In some embodiments, the method comprises deprotecting the pseudo-proline containing GATA3 peptide or derivative thereof.

In some embodiments, the amino acid or derivative thereof to which at least one di-peptide or derivative thereof is coupled is selected from the group consisting of Ala, Cys, Asp, Glu, Phe, Gly, Ile, Lys, Leu, Met, Asn, Pro, Gln, Arg, Ser, Thr, Trp, Tyr, His, and Val. In some embodiments, the one or more selected amino acids, small peptides or derivatives thereof optionally coupled to the pseudo-proline containing GATA3 peptide or derivative thereof comprise Fmoc-Ala-OH.H2O, Fmoc-Cys(Trt)-OH, Fmoc-Asp(OtBu)-OH, Fmoc-Asp(OMpe)-OH, Fmoc-Glu(OtBu)-OH, Fmoc-Phe-OH, Fmoc-Gly-OH, Fmoc-Ile-OH, Fmoc-Lys(Boc)-OH, Fmoc-Leu-OH, Fmoc-Met-OH, Fmoc-Asn(Trt)-OH, Fmoc-Pro-OH, Fmoc-Gln(Trt)-OH, Fmoc-Arg(Pbf)-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-His(Trt)-OH and Fmoc-His(Boc)-OH.

In some embodiments, an N-terminal amino acid or derivative thereof of the GATA3 peptide or derivative thereof is selected from the group consisting of Fmoc-Ala-OH.H2O, Fmoc-Cys(Trt)-OH, Fmoc-Asp(OtBu)-OH, Fmoc-Asp(OMpe)-OH, Fmoc-Glu(OtBu)-OH, Fmoc-Phe-OH, Fmoc-Gly-OH, Fmoc-Ile-OH, Fmoc-Lys(Boc)-OH, Fmoc-Leu-OH, Fmoc-Met-OH, Fmoc-Asn(Trt)-OH, Fmoc-Pro-OH, Fmoc-Gln(Trt)-OH, Fmoc-Arg(Pbf)-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-His(Trt)-OH and Fmoc-His(Boc)-OH.

In some embodiments, the pseudo-proline moiety is (a) Fmoc-Ser(tBu)-Ser(psi(Me,Me)pro)-OH, (b) Fmoc-Ala-Thr(psi(Me,Me)pro)-OH, (c) Fmoc-Glu(OtBu)-Ser(psi(Me,Me)pro)-OH, (d) Fmoc-Leu-Thr(psi(Me,Me)pro)-OH, (e) Fmoc-Leu-Cys(psi(Dmp,H)pro)-OH. In some embodiments, (a) Xaa-Ser is Ser-Ser, (b) Xaa-Ser is Glu-Ser, (c) Xaa-Thr is Ala-Thr, (d) Xaa-Thr is Leu-Thr, or (e) Xaa-Cys is Leu-Cys.

In one aspect provided herein is a method of treating a subject with cancer comprising administering to the subject the pharmaceutical composition of any one of aspects described above.

In one aspect, provided herein is a method of identifying a subject with cancer as a candidate for a therapeutic, the method comprising identifying the subject as one that expresses a protein encoded by an HLA-A02:01 allele, an HLA-A24:02 allele, an HLA-A03:01 allele, an HLA-B07:02 allele and/or an HLA-B08:01 allele, wherein the therapeutic comprises (a) at least one polypeptide comprising one or more mutant GATA3 peptide sequences, wherein each of the one or more mutant GATA3 peptide sequences comprises at least one mutant amino acid and is fragment of at least 8 contiguous amino acids of a mutant GATA3 protein arising from a mutation in a GATA3 gene of a cancer cell; or (b) at least one polynucleotide comprising a sequence encoding the at least one polypeptide, wherein each of the one or more mutant GATA3 peptide sequences or a portion thereof binds to a protein encoded by an HLA-A02:01 allele, an HLA-A24:02 allele, an HLA-A03:01 allele, an HLA-B07:02 allele and/or an HLA-B08:01 allele. In some embodiments, the method further comprises administering the therapeutic to the subject.

In one aspect provided herein is a method of treating a subject with cancer comprising administering to the subject a pharmaceutical composition comprising: (a) at least one polypeptide comprising a first mutant GATA3 peptide sequence and a second mutant GATA3 peptide sequence, wherein (i) the first mutant GATA3 peptide sequence and the second mutant GATA3 peptide sequence each comprise at least 8 contiguous amino acids of SEQ ID NO: 1, and (ii) a C-terminal sequence of the first mutant GATA3 peptide sequence overlaps with an N-terminal sequence of the second mutant GATA3 peptide sequence; wherein the at least 8 contiguous amino acids of SEQ ID NO: 1 comprises at least one amino acid of sequence PGRPLQTHVLPEPHLALQPLQPHADHAHADAPAIQPVLWTTPPLQHGHRHGLEPCSMLTGPPARVPA VPFDLHFCRSSIMKPKRDGYMFLKAESKIMFAT LQRSSLWCLCSNH (SEQ ID NO: 2), or (b) at least one polynucleotide comprising a sequence encoding the at least one polypeptide, wherein HLA alleles expressed by subject are unknown at the time of administering.

In some embodiments, the at least 8 contiguous amino acid of SEQ ID NO: 1 comprises at least one amino acid of sequence:

(SEQ ID NO: 2)
PGRPLQTHVLPEPHLALQPLQPHADHAHADAPAIQPVLWTTPPLQHGHRH
GLEPCSMLTGPPARVPAVPFDLHFCRSSIMKPKRDGYMFLKAESKIMFAT
LQRSSLWCLCSNH.

In some embodiments, the cancer is selected from the group consisting of melanoma, ovarian cancer, lung cancer, prostate cancer, breast cancer, colorectal cancer, endometrial cancer, and chronic lymphocytic leukemia (CLL). In some embodiments, the subject has a breast cancer that is resistant to anti-estrogen therapy, is an MSI breast cancer, is a metastatic breast cancer, is a Her2 negative breast cancer, is a Her2 positive breast cancer, is an ER negative breast cancer, is an ER positive breast cancer, is a PR positive breast cancer, is a PR negetive breast cancer or any combination thereof.

In some embodiments, the breast cancer expresses an estrogen receptor with a mutation. In some embodiments, the method of aspects described above further comprises administering at least one additional therapeutic agent or modality. In some embodiments, the at least one additional therapeutic agent or modality is surgery, a checkpoint inhibitor, an antibody or fragment thereof, a chemotherapeutic agent, radiation, a vaccine, a small molecule, a T cell, a vector, and APC, a polynucleotide, an oncolytic virus or any combination thereof. In some embodiments, the at least one additional therapeutic agent is an anti-PD-1 agent and anti-PD-L1 agent, an anti-CTLA-4 agent, an anti-CD40 agent, letrozole, fulvestrant, a PI3 kinase inhibitor and/or a CDK 4/6 inhibitor. In some embodiments, the at least one additional therapeutic agent is palbociclib, ribociclib, abemaciclib, seliciclib, dinaciclib, milciclib, roniciclib, atuveciclib, briciclib, riviciclib, seliciclib, trilaciclib, voruciclib or any combination thereof.

In some embodiments, the at least one additional therapeutic agent is palbociclib (PD0332991); abemaciclib (LY2835219); ribociclib (LEE 011); voruciclib (P1446A-05); fascaplysin; arcyriaflavin; 2-bromo-12,13-dihydro-5H-indolo[2,3-a]pyrrolo[3,4-c]carbazole-5,7(6H)-dione; 3-amino thioacridone (3-ATA), trans-4-((6-(ethylamino)-2-((1-(phenylmethyl)-1H-indol-5-yl)amino)-4-pyrimidinyl)amino)-cyclohexano (CINK4); 1,4-dimethoxyacridine-9(10H)-thione (NSC 625987); 2-methyl-5-(p-tolylamino)benzo[d]thiazole-4,7-dione (ryuvidine); flavopiridol (alvocidib); seliciclib; dinaciclib; milciclib; roniciclib; atuveciclib; briciclib; riviciclib; trilaciclib (G1T28); or any combination thereof.

In some embodiments, the at least one additional therapeutic agent is Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136.

In some embodiments, the cancer is recurrent or metastatic breast cancer. In some embodiments, the subject is a subject that has had disease progression following endocrine therapy in combination with a CDK 4/6 inhibitor; or wherein the subject has not received prior systemic therapy. In some embodiments, the method comprises determining a mutation status of an estrogen receptor gene of cells of the subject. In some embodiments, the cells are isolated cells or cells enriched for expression of estrogen receptor.

In aspects, provided herein is a composition comprising at least one polypeptide comprising one or more mutant GATA3 peptide sequences, wherein each of the one or more mutant GATA3 peptide sequences comprises at least one mutant amino acid, and is a fragment of at least 8 contiguous amino acids of a mutant GATA3 protein arising from a mutation in a GATA3 gene of a cancer cell; at least one polynucleotide comprising a sequence encoding the at least one polypeptide; one or more APCs comprising the at least one polypeptide; or a T cell receptor (TCR) specific for an neoepitope of the at least one polypeptide in complex with an HLA protein.

In some embodiments, the one or more mutant GATA3 peptide sequences comprises two or more mutant GATA3 peptide sequences. In some embodiments, each of the one or more mutant GATA3 peptide sequence comprises at least 8 contiguous amino acids of SEQ ID NO: 1 or 2.

In aspects, provided herein is a composition comprising at least one polypeptide comprising two or more mutant GATA3 peptide sequences, wherein each of the two or more mutant GATA3 peptide sequences comprise at least 8 contiguous amino acids of SEQ ID NO: 1, and a C-terminal sequence of a first GATA3 peptide sequence overlaps with an N-terminal sequence of a second GATA3 peptide sequence; at least one polynucleotide comprising a sequence encoding the at least one polypeptide one or more APCs comprising the at least one polypeptide; or a T cell receptor (TCR) specific for an neoepitope of the at least one polypeptide in complex with an HLA protein.

In some embodiments, the mutant GATA3 peptide sequences comprise a fragment of a mutant GATA3 protein arising from a frameshift mutation in a GATA3 gene of a cancer cell. In some embodiments, the at least 8 contiguous amino acids comprise at least one amino acid encoded by a GATA3 neoORF sequence. In some embodiments, the mutation in a GATA3 gene of a cancer cell is a frameshift mutation. In some embodiments, the mutation in a GATA3 gene of a cancer cell is a missense mutation, a splice site mutation, or a gene fusion mutation. In some embodiments, each of the mutant GATA3 peptide sequences comprise at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mutant amino acids.

In some embodiments, the at least one polypeptide comprises at least 3, 4, 5, 6, 7, 8, 9, or 10 mutant GATA3 peptide sequences. In some embodiments, the at least one polypeptide comprises at least two polypeptides, or the at least one polynucleotide comprises at least two polynucleotides. In some embodiments, at least one of the one or more GATA3 peptide sequences or at least one of the two or more GATA3 peptide sequences comprises at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a GATA3 protein. In some embodiments, at least two of the GATA3 peptide sequences comprise at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a GATA3 protein.

In some embodiments, each of the GATA3 peptide sequences comprise at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a GATA3 protein. In some embodiments, at least one of the two or more mutant GATA3 peptide sequence comprises at least 8 contiguous amino acids of SEQ ID NO: 2. In some embodiments, at least 3, 4, 5, 6, 7, 8, 9, or 10 of the two or more mutant GATA3 peptide sequence comprises at least 8 contiguous amino acids of SEQ ID NO: 2. In some embodiments, each of one of the two or more mutant GATA3 peptide sequence comprises at least 8 contiguous amino acids of SEQ ID NO: 2. In some embodiments, at least one of the two or more mutant GATA3 peptide sequence comprises at least 8 contiguous amino acids of SEQ ID NO: 3.

In some embodiments, at least one of the at least 8 contiguous amino acids is an amino acid of SEQ ID NO: 4. In some embodiments, a contiguous amino acid of the at least 8 contiguous amino acids is not an amino acid of SEQ ID NO: 4. In some embodiments, the at least one polypeptide comprises at least one mutant GATA3 peptide sequence that binds to or is predicted to bind to a protein encoded by an HLA-A02:01 allele, an HLA-A24:02 allele, an HLA-A03:01 allele, an HLA-B07:02 allele and/or an HLA-B08:01 allele. In some embodiments, the at least one polypeptide comprises at least one mutant GATA3 peptide sequence that binds to or is predicted to bind to a protein encoded by: an HLA-A02:01 allele and an HLA-A24:02 allele; an HLA-A02:01 allele and an HLA-B08:01 allele; an HLA-A24:02 allele and an HLA-B08:01 allele; or an HLA-A02:01 allele, an HLA-A24:02 allele and an HLA-B08:01 allele.

In some embodiments, the two or more mutant GATA3 peptide sequences comprise a first mutant GATA3 peptide sequence that binds to or is predicted to bind to a protein encoded by an HLA-A02:01 allele, an HLA-A24:02 allele, an HLA-A03:01 allele, an HLA-B07:02 allele or an HLA-B08:01 allele; and a second GATA3 peptide sequence that binds to or is predicted to bind to a protein encoded by an HLA-A02:01 allele, an HLA-A24:02 allele, an HLA-A03:01 allele, an HLA-B07:02 allele or an HLA-B08:01 allele; wherein the first mutant GATA3 peptide sequence binds to or is predicted to bind to a protein encoded by different HLA allele than the second mutant GATA3 peptide sequence.

In some embodiments, the at least one polypeptide comprises at least one mutant GATA3 peptide sequence that binds to a protein encoded by an HLA allele with an affinity of less than 10 μM, less than 1 μM, less than 500 nM, less than 400 nM, less than 300 nM, less than 250 nM, less than 200 nM, less than 150 nM, less than 100 nM, or less than 50 nM. In some embodiments, the at least one polypeptide comprises at least one mutant GATA3 peptide sequence that binds to a protein encoded by an HLA allele with a stability of greater than 24 hours, greater than 12 hours, greater than 9 hours, greater than 6 hours, greater than 5 hours, greater than 4 hours, greater than 3 hours, greater than 2 hours, greater than 1 hour, greater than 45 minutes, greater than 30 minutes, greater than 15 minutes, or greater than 10 minutes. In some embodiments, the HLA allele is selected from the group consisting of HLA-A02:01, HLA-A24:02, HLA-A03:01, HLA-B07:02, HLA-B08:01 and any combination thereof.

In some embodiments, the at least one polypeptide comprises at least one of the following sequences: TLQRSSLWCL, VLPEPHLAL, HVLPEPHLAL, ALQPLQPHA, AIQPVLWTT, APAIQPVLWTT, SMLTGPPARV, MLTGPPARV, and/or YMFLKAESKI; and/or MFLKAESKI and/or YMFLKAESKI VLWTTPPLQH, YMFLKAESK and/or KIMFATLQR; and/or FATLQRSSL, EPHLALQPL, QPVLWTTPPL, GPPARVPAV, MFATLQRSSL KPKRDGYMF and/or KPKRDGYMFL and/or IMKPKRDGYM, MFATLQRSSL, FLKAESKIMF, LHFCRSSIM, EPHLALQPL, FATLQRSSL, ESKIMFATL, FLKAESKIM and/or YMFLKAESKI.

In some embodiments, the two or more mutant GATA3 peptide sequences comprise at least two of the following sequences: TLQRSSLWCL, VLPEPHLAL, HVLPEPHLAL, ALQPLQPHA, AIQPVLWTT, APAIQPVLWTT, SMLTGPPARV, MLTGPPARV, and/or YMFLKAESKI; and/or MFLKAESKI and/or YMFLKAESKI VLWTTPPLQH, YMFLKAESK and/or KIMFATLQR; and/or FATLQRSSL, EPHLALQPL, QPVLWTTPPL, GPPARVPAV, MFATLQRSSL KPKRDGYMF and/or KPKRDGYMFL and/or IMKPKRDGYM, MFATLQRSSL, FLKAESKIMF, LHFCRSSIM EPHLALQPL, FATLQRSSL, ESKIMFATL, FLKAESKIM and/or YMFLKAESKI.

In some embodiments, the mutant GATA3 peptide sequences comprise at least two of the following sequences EPCSMLTGPPARVPAVPFDLH, SMLTGPPARVPAVPFDLH, GPPARVPAVPFDLHFCRSSIMKPKRD, DLHFCRSSIMKPKRDGYMFLKAESKI, KPKRDGYMFLKAESKIMFATLQRSSLWCLCSNH, FLKAESKIMFATLQRS, and KPKRDGYMFLKAESKI.

In some embodiments, the mutant GATA3 peptide sequences comprise at least two sequences of Table 5 and/or Table 6. In some embodiments, a first mutant GATA3 peptide sequence of the two or more mutant GATA3 peptide sequence comprises a first neoepitope of GATA3 protein and a second peptide mutant GATA3 peptide sequence comprises a second neoepitope of a mutant GATA protein, wherein the first mutant GATA3 peptide sequence is different from the mutant GATA3 peptide sequence, and wherein the first neoepitope comprises at least one mutant amino acid and the second neoepitope comprises the same mutant amino acid.

In aspects, provided herein is a composition comprising at least one polypeptide comprising one or more mutant GATA3 peptide sequences, wherein the at least one polypeptide is represented by a formula of

[Xaa]F-[Xaa]N-[Xaa]C, wherein each Xaa is independently any amino acid, wherein [Xaa]N-[Xaa]C represents the one or more mutant GATA3 peptide sequences, wherein [Xaa]N and [Xaa]C each comprise a contiguous amino acid sequence encoded by a different portion of the GATA3 gene, wherein [Xaa]N is encoded in a non-wild type reading frame, wherein [Xaa]C comprises the at least one mutant amino acid and is encoded in a non-wild type reading frame, wherein N is an integer of from 0-100, wherein C is an integer of from 1-100, wherein F is an integer of from 0-100, wherein the sum of N and M is at least 8.

In some embodiments, each of the mutant GATA3 peptide sequences the at least eight contiguous amino acids are represented by a formula of [Xaa]F-[Xaa]N-[Xaa]C or [Xaa]N-[Xaa]C-[Xaa]F, wherein each Xaa is an amino acid, wherein [Xaa]N and [Xaa]C each comprise an amino acid sequence encoded by a different portion of the GATA3 gene, wherein [Xaa]F is any amino acid sequence, wherein [Xaa]N is encoded in a non-wild type reading frame of the GATA3 gene, wherein [Xaa]C comprises the at least one mutant amino acid and is encoded in a non-wild type reading frame of the GATA3 gene, wherein N is an integer of from 0-100, wherein C is an integer of from 1-100, wherein F is an integer of from 0-100, wherein the sum of N and M is at least 8. In some embodiments, each Xaa of [Xaa]F is a lysine residue and F is an integer of from 1-100, 1-10, 9, 8, 7, 6, 5, 4, 3, 2 or 1. In some embodiments, F is 3, 4 or 5.

In some embodiments, the at least one mutant amino acid comprises at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous mutant amino acids. In some embodiments, each of the mutant GATA3 peptide sequences are present at a concentration at least 1 μg/mL, at least 10 μg/mL, at least 25 μg/mL, at least 50 μg/mL, at least 100 μg/mL, at least 200 μg/mL, at least 250 μg/mL, at least 300 μg/mL or at least 400 μg/mL. In some embodiments, each of the mutant GATA3 peptide sequences are present at a concentration at most 5000 μg/mL, at most 2500 μg/mL, at most 1000 μg/mL, at most 750 μg/mL, at most 500 μg/mL, at most 400 μg/mL, or at most 300 μg/mL. In some embodiments, each of the mutant GATA3 peptide sequences are present at a concentration of from 10 g/mL to 5000 μg/mL, 10 μg/mL to 4000 μg/mL, 10 μg/mL to 3000 μg/mL, 10 μg/mL to 2000 μg/mL, 10 g/mL to 1000 μg/mL, 25 μg/mL to 500 μg/mL, 50 μg/mL to 500 μg/mL, 100 μg/mL to 500 μg/mL, 200 g/mL to 500 μg/mL, 200 μg/mL to 400 μg/mL or 3000 μg/mL to 400 μg/mL.

In some embodiments, the composition further comprising an immunomodulatory agent or an adjuvant. In some embodiments, the adjuvant is polyICLC. In aspects, provided herein is a pharmaceutical composition comprising a composition described herein, and a pharmaceutically acceptable excipient. In some embodiments, the pharmaceutical composition comprises a pH modifier present at a concentration of less than 1 mM or greater than 1 mM. In some embodiments, the pharmaceutical composition is a vaccine composition. In some embodiments, the pharmaceutical composition is aqueous.

In some embodiments, one or more of the at least one polypeptide is bounded by pI>5 and HYDRO>−6, pI>8 and HYDRO>−8, pI<5 and HYDRO>−5, pI>9 and HYDRO<−8, pI>7 and a HYDRO value of >−5.5, pI<4.3 and −4≥HYDRO≥−8, pI>0 and HYDRO<−8, pI>0 and HYDRO>−4, or pI>4.3 and −4≥HYDRO≥−8, pI>0 and HYDRO>−4, or pI>4.3 and HYDRO≤−4, pI>0 and HYDRO>−4, or pI>4.3 and −4≥HYDRO≥−9, 5≥pI≥12 and −4≥HYDRO≥−9.

In some embodiments, the pH modifier is a base. In some embodiments, the pH modifier is a conjugate base of a weak acid. In some embodiments, the pH modifier is a pharmaceutically acceptable salt. In some embodiments, the pH modifier is a dicarboxylate or tricarboxylate salt. In some embodiments, the pH modifier is citric acid and/or a citrate salt. In some embodiments, the citrate salt is disodium citrate and/or trisodium citrate. In some embodiments, the pH modifier is succinic acid and/or a succinate salt. In some embodiments, the succinate salt is a disodium succinate and/or a monosodium succinate. In some embodiments, the succinate salt is disodium succinate hexahydrate. In some embodiments, the pH modifier is present at a concentration of from 0.1 mM-10 mM. In some embodiments, the pH modifier is present at a concentration of from 0.1 mM-5 mM. In some embodiments, the pH modifier is present at a concentration of from 0.1 mM-1 mM. In some embodiments, the pH modifier is present at a concentration of from 1 mM-10 mM. In some embodiments, the pH modifier is present at a concentration of from 1 mM-5 mM.

In some embodiments, the pharmaceutically acceptable carrier comprises a liquid. In some embodiments, the pharmaceutically acceptable carrier comprises water. In some embodiments, the pharmaceutically acceptable carrier comprises a sugar. In some embodiments, the sugar comprises dextrose or mannitol. In some embodiments, the dextrose or mannitol is present at a concentration of from 1-10% w/v. In some embodiments, the sugar comprises trehalose. In some embodiments, the sugar comprises sucrose. In some embodiments, the pharmaceutically acceptable carrier comprises dimethyl sulfoxide (DMSO).

In some embodiments, the DMSO is present at a concentration from 0.1% to 10%, 0.5% to 5%, 1% to 5%, 2% to 5%, 2% to 4%, or 2% to 4%. In some embodiments, the pharmaceutically acceptable carrier does not comprise dimethyl sulfoxide (DMSO). In some embodiments, the pharmaceutical composition is lyophilizable. In some embodiments, the pharmaceutical composition further comprises an immunomodulator or adjuvant. In some embodiments, the immunomodulator or adjuvant is selected from the group consisting of poly-ICLC, 1018 ISS, aluminum salts, Amplivax, AS15, BCG, CP-870,893, CpG7909, CyaA, ARNAX, STING agonists, dSLIM, GM-CSF, IC30, IC31, Imiquimod, ImuFact IMP321, IS Patch, ISS, ISCOMATRIX, JuvImmune, LipoVac, MF59, monophosphoryllipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, resiquimod, SRL172, Virosomes and other Virus-like particles, YF-17D, VEGF trap, R848, beta-glucan, Pam3Cys, and Aquila's QS21 stimulon.

In some embodiments, the immunomodulator or adjuvant comprises poly-ICLC. In some embodiments, a ratio of poly-ICLC to peptides in the pharmaceutical composition is from 2:1 to 1:10 v:v. In some embodiments, the ratio of poly-ICLC to peptides in the pharmaceutical composition is about 1:1, 1:1.5, 1:2, 1:3, 1:4 or 1:5 v:v. In some embodiments, the ratio of poly-ICLC to peptides in the pharmaceutical composition is about 1:3 v:v.

In aspects, provided herein is a method of synthesizing a GATA3 peptide, wherein the peptide comprises a sequence of at least two contiguous amino acids selected from the group consisting of Xaa-Cys, Xaa-Ser, and Xaa-Thr, wherein Xaa is any amino acid, the method comprising: coupling at least one di-peptide or derivative thereof to an amino acid or derivative thereof of a GATA3 peptide or derivative thereof to obtain a pseudo-proline containing GATA3 peptide or derivative thereof, wherein the di-peptide or derivative thereof comprises a pseudo-proline moiety; coupling one or more selected amino acids, small peptides or derivatives thereof to the pseudo-proline containing GATA3 peptide or derivative thereof, and cleaving the pseudo-proline containing GATA3 peptide or derivative thereof from the resin.

In some embodiments, the method comprises deprotecting the pseudo-proline containing GATA3 peptide or derivative thereof. In some embodiments, the GATA3 peptide is a peptide of the at least one polypeptide of a composition described herein or of the pharmaceutical composition herein. In some embodiments, an N-terminal amino acid or derivative thereof of the GATA3 peptide or derivative thereof is attached to a resin. In some embodiments, the resin is a Wang resin or a 2-chlorotrityl resin (2-Cl-Trt resin). In some embodiments, a starting material for the coupling is Fmoc-His(Trt)-Wang resin, H-His(Trt)-2Cl-Trt resin, Fmoc-Asp(OtBu)-Wang resin, Fmoc-Ile-Wang resin, Fmoc-Ser(tBu)-Wang resin, or Fmoc-Leu-Wang resin. In some embodiments, the amino acid or derivative thereof to which at least one di-peptide or derivative thereof is coupled is selected from the group consisting of Ala, Cys, Asp, Glu, Phe, Gly, Ile, Lys, Leu, Met, Asn, Pro, Gln, Arg, Ser, Thr, Trp, Tyr, His, and Val.

In some embodiments, the one or more selected amino acids, small peptides or derivatives thereof optionally coupled to the pseudo-proline containing GATA3 peptide or derivative thereof comprise Fmoc-Ala-OH.H2O, Fmoc-Cys(Trt)-OH, Fmoc-Asp(OtBu)-OH, Fmoc-Asp(OMpe)-OH, Fmoc-Glu(OtBu)-OH, Fmoc-Phe-OH, Fmoc-Gly-OH, Fmoc-Ile-OH, Fmoc-Lys(Boc)-OH, Fmoc-Leu-OH, Fmoc-Met-OH, Fmoc-Asn(Trt)-OH, Fmoc-Pro-OH, Fmoc-Gln(Trt)-OH, Fmoc-Arg(Pbf)-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-His(Trt)-OH and Fmoc-His(Boc)-OH.

In some embodiments, an N-terminal amino acid or derivative thereof of the GATA3 peptide or derivative thereof is selected from the group consisting of Fmoc-Ala-OH.H2O, Fmoc-Cys(Trt)-OH, Fmoc-Asp(OtBu)-OH, Fmoc-Asp(OMpe)-OH, Fmoc-Glu(OtBu)-OH, Fmoc-Phe-OH, Fmoc-Gly-OH, Fmoc-Ile-OH, Fmoc-Lys(Boc)-OH, Fmoc-Leu-OH, Fmoc-Met-OH, Fmoc-Asn(Trt)-OH, Fmoc-Pro-OH, Fmoc-Gln(Trt)-OH, Fmoc-Arg(Pbf)-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-His(Trt)-OH and Fmoc-His(Boc)-OH.

In some embodiments, the pseudo-proline moiety is Fmoc-Ser(tBu)-Ser(psi(Me,Me)pro)-OH. In some embodiments, the pseudo-proline moiety is Fmoc-Ala-Thr(psi(Me,Me)pro)-OH. In some embodiments, the pseudo-proline moiety is Fmoc-Glu(OtBu)-Ser(psi(Me,Me)pro)-OH. In some embodiments, the pseudo-proline moiety is Fmoc-Leu-Thr(psi(Me,Me)pro)-OH. In some embodiments, the pseudo-proline moiety is Fmoc-Leu-Cys(psi(Dmp,H)pro)-OH.

In some embodiments, Xaa-Ser is Ser-Ser. In some embodiments, Xaa-Ser is Glu-Ser. In some embodiments, Xaa-Thr is Ala-Thr. In some embodiments, Xaa-Thr is Leu-Thr. In some embodiments, Xaa-Cys is Leu-Cys.

In aspects, provided herein is a method of treating a subject with cancer comprising administering to the subject a pharmaceutical composition described herein.

In aspects, provided herein is a method of identifying a subject with cancer as a candidate for a therapeutic, the method comprising identifying the subject as one that expresses a protein encoded by an HLA-A02:01 allele, an HLA-A24:02 allele, an HLA-A03:01 allele, an HLA-B07:02 allele and/or an HLA-B08:01 allele, wherein the therapeutic comprises at least one polypeptide comprising one or more mutant GATA3 peptide sequences, wherein each of the one or more mutant GATA3 peptide sequences comprises at least one mutant amino acid and is fragment of at least 8 contiguous amino acids of a mutant GATA3 protein arising from a mutation in a GATA3 gene of a cancer cell; at least one polynucleotide comprising a sequence encoding the at least one polypeptide; one or more APCs comprising the at least one polypeptide; or a T cell receptor (TCR) specific for an neoepitope of the at least one polypeptide in complex with an HLA protein; wherein each of the one or more mutant GATA3 peptide sequences or a portion thereof binds to a protein encoded by an HLA-A02:01 allele, an HLA-A24:02 allele, an HLA-A03:01 allele, an HLA-B07:02 allele and/or an HLA-B08:01 allele. In some embodiments, the method further comprises administering the therapeutic to the subject.

In aspects, provided herein is a method of treating a subject with cancer comprising administering to the subject a composition comprising: at least one polypeptide comprising one or more mutant GATA3 peptide sequences, wherein each of the one or more mutant GATA3 peptide sequences comprises at least one mutant amino acid and is fragment of at least 8 contiguous amino acids of a mutant GATA3 protein arising from a mutation in a GATA3 gene of a cancer cell; at least one polynucleotide comprising a sequence encoding the at least one polypeptide; one or more APCs comprising the at least one polypeptide; or a T cell receptor (TCR) specific for an neoepitope of the at least one polypeptide in complex with an HLA protein; wherein the mutant GATA3 peptide or as portion thereof binds to a protein encoded by an HLA-A02:01 allele, an HLA-A24:02 allele, an HLA-A03:01 allele, an HLA-B07:02 allele and/or an HLA-B08:01 allele; wherein the subject is identified as expressing an HLA-A02:01 allele, an HLA-A24:02 allele, an HLA-A03:01 allele, an HLA-B07:02 allele and/or an HLA-B08:01 allele.

In aspects, provided herein is a method of treating a subject with cancer comprising administering to the subject a composition comprising at least one polypeptide comprising two or more mutant GATA3 peptide sequences, wherein each of the two or more mutant GATA3 peptide sequence comprises at least 8 contiguous amino acids of SEQ ID NO: 1, and a C-terminal sequence of a first GATA3 peptide sequence overlaps with an N-terminal sequence of a second GATA3 peptide sequence; at least one polynucleotide comprising a sequence encoding the at least one polypeptide; one or more APCs comprising the at least one polypeptide; or a T cell receptor (TCR) specific for an neoepitope of the at least one polypeptide in complex with an HLA protein; wherein HLA alleles expressed by subject are unknown at the time of administering.

In some embodiments, an immune response is elicited in the subject. In some embodiments, the immune response is a humoral response. In some embodiments, the mutant GATA3 peptide sequences are administered simultaneously, separately or sequentially. In some embodiments, the first peptide is sequentially administered after a time period sufficient for the second peptide to activate the second T cells. In some embodiments, the cancer is selected from the group consisting of melanoma, ovarian cancer, lung cancer, prostate cancer, breast cancer, colorectal cancer, endometrial cancer, and chronic lymphocytic leukemia (CLL). In some embodiments, the subject has a breast cancer that is resistant to anti-estrogen therapy, is an MSI breast cancer, is a metastatic breast cancer, is a Her2 negative breast cancer, is a Her2 positive breast cancer, is an ER negative breast cancer, is an ER positive breast cancer, is a PR positive breast cancer, is a PR negetive breast cancer or any combination thereof. In some embodiments, the breast cancer expresses an estrogen receptor with a mutation. In some embodiments, the method further comprises administering at least one additional therapeutic agent or modality.

In some embodiments, the at least one additional therapeutic agent or modality is surgery, a checkpoint inhibitor, an antibody or fragment thereof, a chemotherapeutic agent, radiation, a vaccine, a small molecule, a T cell, a vector, and APC, a polynucleotide, an oncolytic virus or any combination thereof. In some embodiments, the at least one additional therapeutic agent is an anti-PD-1 agent and anti-PD-L1 agent, an anti-CTLA-4 agent, an anti-CD40 agent, letrozole, fulvestrant, and/or a CDK 4/6 inhibitor. In some embodiments, the at least one additional therapeutic agent is selected from the group consisting of palbociclib (PD0332991); abemaciclib (LY2835219); ribociclib (LEE 011); voruciclib (P1446A-05); fascaplysin; arcyriaflavin; 2-bromo-12,13-dihydro-5H-indolo[2,3-a]pyrrolo[3,4-c]carbazole-5,7(6H)-dione; 3-amino thioacridone (3-ATA), trans-4-((6-(ethylamino)-2-((1-(phenylmethyl)-1H-indol-5-yl)amino)-4-pyrimidinyl)amino)-cyclohexano (CINK4); 1,4-dimethoxyacridine-9(10H)-thione (NSC 625987); 2-methyl-5-(p-tolylamino)benzo[d]thiazole-4,7-dione (ryuvidine); and flavopiridol (alvocidib); seliciclib; dinaciclib; milciclib; roniciclib; atuveciclib; briciclib; riviciclib; trilaciclib (G1T28); and any combination thereof.

In some embodiments, the additional therapeutic agent is administered before, simultaneously, or after administering the mutant GATA3 peptide sequences. In some embodiments, administering comprises administering subcutaneously or intravenously. In some embodiments, the cancer is recurrent or metastatic breast cancer. In some embodiments, the subject is a subject that has had disease progression following endocrine therapy in combination with a CDK 4/6 inhibitor.

A mutation common for CLL and certain lymphomas is a Cysteine to Serine change at position 481 (C481S) in the BTK (Bruton's Tyrosine Kinase) gene. The mutation is harbored in a region having the amino acid sequence: IFIITEYMANGSLLNYLREMRHR, the mutated Serine is underlined. This change produces a number of binding peptides which bind to a range of HLA molecules.

In one aspect, provided herein is a composition comprising a polypeptide, comprising one or more mutant BTK peptide sequences from a C481S mutant BTK protein, the one or more mutant BTK peptide sequences comprising at least 8 contiguous amino acids of the mutant BTK protein, wherein the amino acid sequences of the peptides are: ANGSLLNY; ANGSLLNYL; ANGSLLNYLR; EYMANGSL; EYMANGSLLN; EYMANGSLLNY; GSLLNYLR; GSLLNYLREM; ITEYMANGS; ITEYMANGSL; ITEYMANGSLL; MANGSLLNYL; MANGSLLNYLR; NGSLLNYL; NGSLLNYL; SLLNYLREMR; TEYMANGSLL; TEYMANGSLLNY; YMANGSLL; or YMANGSLLN, listed in Table 34.

In some embodiments, the one or more mutant BTK peptide sequences comprise: (a) ANGSLLNY and binds to or is predicted to bind to a protein encoded by an HLA-A36:01 allele, (b) ANGSLLNYL and binds to or is predicted to bind to a protein encoded by an HLA allele selected from a group consisting of HLA-C15:02, HLA-C08:01, HLA-C06:02, HLA-A02:04, HLA-C12:02, HLA-B44:02, HLA-C17:01 and HLA-B38:01, (c) ANGSLLNYLR and binds to or is predicted to bind to a protein encoded by an HLA-A74:01 allele, or an HLA-A31:01 allele, (d) EYMANGSL and binds to or is predicted to bind to a protein encoded by an HLA allele selected from a group consisting of HLA-C14:02, HLA-C14:03 and HLA-A24:02, (e) EYMANGSLLN and binds to or is predicted to bind to a protein encoded by an HLA-A24:02 allele or an HLA-A23:01 allele, (f) EYMANGSLLNY and binds to or is predicted to bind to a protein encoded by an HLA-A29:02 allele, (g) GSLLNYLR and binds to or is predicted to bind to a protein encoded by an HLA-A31:01 allele or an HLA-A74:01 allele, (h) GSLLNYLREM and binds to or is predicted to bind to a protein encoded by an HLA-B58:02 allele or an HLA-B57:01 allele, (i) ITEYMANGS and binds to or is predicted to bind to a protein encoded by an HLA-A01:01 allele, (j) ITEYMANGSL and binds to or is predicted to bind to a protein encoded by an HLA-A01:01 allele, (k) ITEYMANGSLL and binds to or is predicted to bind to a protein encoded by an HLA-A01:01 allele, (1) MANGSLLNYL and binds to or is predicted to bind to a protein encoded by an HLA allele selected from a group consisting of HLA-C17:01, HLA-C02:02, HLA-B35:01, HLA-C03:03, HLA-C08:01, HLA-B35:03, HLA-C12:02, HLA-C01:02, HLA-C03:04 and HLA-C08:02, (m) MANGSLLNYLR and binds to or is predicted to bind to a protein encoded by an HLA-A33:03 allele or an HLA-A74:01 allele, (n) NGSLLNYL and binds to or is predicted to bind to a protein encoded by an HLA-B14:02 allele, (o) NGSLLNYL and binds to or is predicted to bind to a protein encoded by an HLA allele selected from a group consisting of: HLA-A68:01, HLA-A33:03, HLA-A31:01 and HLA-A74:01, (p) SLLNYLREMR and binds to or is predicted to bind to a protein encoded by an HLA-A74:01 allele or an HLA-A31:01 allele, (q) TEYMANGSLL and binds to or is predicted to bind to a protein encoded by an HLA allele selected from a group consisting of: HLA-B40:01, HLA-B44:03, HLA-B49:01, HLA-B44:02 and HLA-B40:02, (r) TEYMANGSLLNY and binds to or is predicted to bind to a protein encoded by an HLA-B44:03 allele, (s) YMANGSLL and binds to or is predicted to bind to a protein encoded by an HLA allele selected from a group consisting of HLA-B15:09, HLA-C03:04, HLA-C03:03, HLA-C17:01, HLA-C03:02, HLA-C14:03, HLA-C14:02, HLA-C04:01, HLA-C02:02, HLA-A01:01, or (t) YMANGSLLN and binds to or is predicted to bind to a protein encoded by an HLA-A29:02 allele or an HLA-A01:01 allele.

In some embodiments, the one or more mutant BTK peptide sequences is specific for a cognate T cell receptor in complex with an HLA protein. In some embodiments, the composition comprises two or more mutant BTK peptide sequences.

In one aspect, provided herein is a composition comprising: at least one polypeptide comprising one or more mutant BTK peptide sequences, each having at least 8 contiguous amino acids from a C481S mutant BTK protein, the one or more mutant BTK peptide sequences selected from Table 34, further comprising three or more amino acid residues that are heterologous to the mutant BTK protein, linked to the N-terminus or C-terminus of a mutant BTK peptide sequence, wherein the three or more amino acid residues enhance processing of the mutant BTK peptide sequences inside a cell and/or enhance presentation of an epitope of the mutant BTK peptide sequences. In some embodiments, the three or more amino acid residues that are heterologous to the mutant BTK protein comprise an amino acid sequence from CMV-pp65, HIV, MART-1 or a non-viral, non-BTK endogenous peptide.

In some embodiments, the three or more amino acid residues that are heterologous to the mutant BTK protein comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

In some embodiments, the three or more amino acid residues that are heterologous to the mutant BTK protein comprise at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, or 100 amino acids.

In one aspect, provided herein is a composition comprising: at least one polypeptide of the formula (N-terminal Xaa)_{N}-(Xaa_{BTK})_{P}-(Xaa-C terminal)_{C} wherein, P is an integer greater than 7; (Xaa_{BTK})_{P} is a mutant BTK peptide sequence comprising at least 8 contiguous amino acids selected from the sequence IFIITEYMANGSLLNYLREMRHR of a mutant BTK protein comprising the C481S mutant amino acid; N is (i) 0 or (ii) an integer greater than 2; (N-terminal Xaa)_{N} is any amino acid sequence heterologous to the mutant BTK protein; C is (i) 0 or (ii) an integer greater than 2; (Xaa-C terminal)_{C} is any amino acid sequence heterologous to the mutant BTK protein; and both N and C are 0.

In some embodiments, the (N-terminal Xaa)_{N} and/or (Xaa-C terminal)_{C} comprises an amino acid sequence of a CMV-pp65, HIV, MART-1 or a non-viral, non-BTK endogenous protein or peptide.

In some embodiments, the N and/or C is an integer greater than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

In some embodiments, the N and/or C is an integer less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, or 100. In some embodiments, the composition of any one of claims 8-10, wherein N is 0. In some embodiments, the 8-10, wherein C is 0.

In one aspect, provided herein is a composition comprising a polynucleotide sequence encoding the polypeptide of claim 1. In one aspect, the composition comprises a polynucleotide sequence encoding one or more peptide sequences of any of the mutant BTK peptides described above, and in Tables 34 and Table 36. In some embodiments, the at least one polypeptide comprises at least 3, 4, 5, 6, 7, 8, 9, or 10 mutant BTK peptide sequences. In some embodiments, the at least one of the mutant BTK peptide sequences comprises at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a mutant BTK protein. In some embodiments, at least 2, 3, 4, 5, 6, 7, 8, 9, or 10 of the mutant BTK peptide sequences comprise at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a mutant BTK protein. In some embodiments, the each of the mutant BTK peptide sequences or each of the two or more BTK peptide sequences comprises at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a mutant BTK protein. In some embodiments, the at least one polypeptide comprises at least one mutant BTK peptide sequence that binds to or is predicted to bind to a protein encoded by an HLA allele listed in Table 35 with an affinity of 150 nM or less and/or a half-life of 2 hours or more. In some embodiments, the mutant BTK peptide sequences comprises (a) a first mutant BTK peptide sequence selected from Table 34 and binds to or is predicted to bind to a protein encoded by an HLA allele; and (b) a second BTK peptide having a C481S mutation, wherein the first mutant BTK peptide sequence and the second mutant BTK peptide sequence are non-identical.

In some embodiments, the at least one polypeptide comprises at least one mutant BTK peptide sequence that binds to a protein encoded by an HLA allele with an affinity of less than 10 μM, less than 1 μM, less than 500 nM, less than 400 nM, less than 300 nM, less than 250 nM, less than 200 nM, less than 150 nM, less than 100 nM, or less than 50 nM.

In some embodiments, the at least one polypeptide comprises at least one mutant BTK peptide sequence that binds to a protein encoded by an HLA allele with a stability of greater than 24 hours, greater than 12 hours, greater than 9 hours, greater than 6 hours, greater than 5 hours, greater than 4 hours, greater than 3 hours, greater than 2 hours, greater than 1 hour, greater than 45 minutes, greater than 30 minutes, greater than 15 minutes, or greater than 10 minutes.

In some embodiments, the (N-terminal Xaa)_{N} comprises an amino acid sequence of IDIIMKIRNA, FFFFFFFFFFFFFFFFFFFFIIFFIFFWMC, FFFFFFFFFFFFFFFFFFFFFFFFAAFWFW, IFFIFFIIFFFFFFFFFFFFIIIIIIIWEC, FIFFFIIFFFFFIFFFFFIFIIIIIIFWEC, TEY, WQAGILAR, HSYTTAE, PLTEEKIK, GALHFKPGSR, RRANKDATAE, KAFISHEEKR, TDLSSRFSKS, FDLGGGTFDV, CLLLHYSVSK, or MTEYKLVVV. In some embodiments, the (C-terminal Xaa)_{C} comprises an amino acid sequence of KKNKKDDIKD, AGNDDDDDDDDDDDDDDDDDKKDKDDDDDD, AGNKKKKKKKNNNNNNNNNNNN NNNNN, AGRDDDDDDDDDDDDDDDDDDDDDDDDDDD, GKSALTIQL, GKSALTI, QGQNLKYQ, ILGVLLLI, EKEGKISK, AASDFIFLVT, KELKQVASPF, KKKLINEKKE, KKCDISLQFF, KSTAGDTHLG, ATFYVAVTVP, LTIQLIQNHFVDEYDPTIEDSYRKQVVIDG, or TIQLIQNHFVDEYDPTIEDSYRKQVVIDGE.

In some embodiments, the at least one of the mutant BTK peptide sequences comprises a mutant amino acid not encoded by the genome of a cancer cell of a subject.

In some embodiments, each of the mutant BTK peptide sequences are present at a concentration at least 1 μg/mL, at least 10 μg/mL, at least 25 μg/mL, at least 50 μg/mL, or at least 100 μg/mL. In some embodiments, the each of the mutant BTK peptide sequences are present at a concentration at most 5000 g/mL, at most 2500 μg/mL, at most 1000 μg/mL, at most 750 μg/mL, at most 500 μg/mL, at most 400 g/mL, or at most 300 μg/mL. In some embodiments, the each of the mutant BTK peptide sequences are present at a concentration of from 10 μg/mL to 5000 μg/mL, 10 μg/mL to 4000 μg/mL, 10 μg/mL to 3000 g/mL, 10 μg/mL to 2000 μg/mL, 10 μg/mL to 1000 μg/mL, 25 μg/mL to 500 μg/mL, or 50 μg/mL to 300 g/mL. In some embodiments, the composition further comprises an immunomodulatory agent or an adjuvant. In some embodiments, the adjuvant is polyICLC.

In one aspect, provided herein is a pharmaceutical composition comprising: (a) the composition described above, and (b) a pharmaceutically acceptable excipient. In some embodiments, the pharmaceutical composition further comprises a pH modifier. In some embodiments, the pharmaceutical composition is a vaccine composition. In some embodiments, the pharmaceutical composition is aqueous. In some embodiments, the pharmaceutical composition comprises the one or more of the at least one polypeptide is bounded by (a) pI>5 and HYDRO>−6, (b) pI>8 and HYDRO>−8, (c) pI<5 and HYDRO>−5, (d) pI>9 and HYDRO<−8, (e) pI>7 and a HYDRO value of >−5.5, (f) pI<4.3 and −4≥HYDRO≥−8, (g) pI>0 and HYDRO≤−8, pI>0 and HYDRO>−4, or pI>4.3 and −4≥HYDRO≥−8, (h) pI>0 and HYDRO>−4, or pI>4.3 and HYDRO≤−4, (i) pI>0 and HYDRO>−4, or pI>4.3 and −4≥HYDRO≥−9, (j) 5>pI>12 and −4≥HYDRO≥−9.

In some embodiments, the pH modifier is a base. In some embodiments, the pH modifier is a conjugate base of a weak acid. In some embodiments, the pH modifier is a pharmaceutically acceptable salt. In some embodiments, the pH modifier is a dicarboxylate or tricarboxylate salt. In some embodiments, the pH modifier is citric acid and/or a citrate salt. In some embodiments, the citrate salt is disodium citrate and/or trisodium citrate. In some embodiments, the pH modifier is succinic acid and/or a succinate salt. In some embodiments, the succinate salt is a disodium succinate and/or a monosodium succinate. In some embodiments, wherein the succinate salt is disodium succinate hexahydrate. In some embodiments, the pH modifier is present at a concentration of from 0.1 mM-1 mM. In some embodiments, the pharmaceutically acceptable carrier comprises a liquid. In some embodiments, the pharmaceutically acceptable carrier comprises water.

In some embodiments, the pharmaceutically acceptable carrier comprises a sugar. In some embodiments, the sugar comprises dextrose. In some embodiments, the dextrose is present at a concentration of from 1-10% w/v. In some embodiments, the sugar comprises trehalose. In some embodiments, the sugar comprises sucrose.

In some embodiments, the pharmaceutically acceptable carrier comprises dimethyl sulfoxide (DMSO). In some embodiments, the DMSO is present at a concentration from 0.1% to 10%, 0.5% to 5%, or 1% to 3%. In some embodiments, the pharmaceutically acceptable carrier does not comprise dimethyl sulfoxide (DMSO). In some embodiments, the pharmaceutical composition is lyophilizable. In some embodiments, the pharmaceutical composition further comprises an immunomodulator or adjuvant. In some embodiments, wherein the immunomodulator or adjuvant is selected from the group consisting of poly-ICLC, 1018 ISS, aluminum salts, Amplivax, AS15, BCG, CP-870,893, CpG7909, CyaA, ARNAX, STING agonists, dSLIM, GM-CSF, IC30, IC31, Imiquimod, ImuFact IMP321, IS Patch, ISS, ISCOMATRIX, JuvImmune, LipoVac, MF59, monophosphoryllipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, resiquimod, SRL172, Virosomes and other Virus-like particles, YF-17D, VEGF trap, R848, beta-glucan, Pam3Cys, and Aquila's QS21 stimulon.

In some embodiments, the immunomodulator or adjuvant comprises poly-ICLC. In some embodiments, a ratio of poly-ICLC to peptides in the pharmaceutical composition is from 2:1 to 1:10 v:v. In some embodiments, the ratio of poly-ICLC to peptides in the pharmaceutical composition is about 1:1, 1:2, 1:3, 1:4 or 1:5 v:v. In some embodiments, the ratio of poly-ICLC to peptides in the pharmaceutical composition is about 1:3 v:v.

In one aspect, provided herein is a method of treating a cancer in a subject, comprising administering to the subject the pharmaceutical composition as described above.

In one aspect, provided herein is a method of treating a cancer in a subject, the method comprising: administering to the subject in need thereof a composition comprising a peptide having a sequence selected from Table 34, 36 or 37 left column; wherein the subject expresses a protein encoded by any one of HLA alleles listed in the right column corresponding to the peptide within the table. In some embodiments, the invention provides a method of treating cancer in a subject, comprising: administering to the subject in need thereof, a composition comprising one or more mutant BTK peptides, or one or more nucleic acids encoding the one or more mutant BTK peptides, wherein each mutant BTK peptide comprises at least 8 contiguous amino acids of a mutant BTK protein comprising a mutation C481S, wherein at least one of the one or more peptides binds to a protein encoded by an HLA allele listed in Table 34, 36 or 37, which is expressed by the subject. In some embodiments, the peptide binds to HLA protein with an affinity of 150 nM or less and/or a half-life of 2 hours or more.

In one aspect, provided herein is a method of treating a cancer in a subject, comprising administering to the subject in need thereof, a first and a second peptide or a nucleic acid encoding the first and the second peptide, wherein the first peptide has an amino acid sequence selected from: Tables 34, 36 or 37; and the second peptide has an amino acid sequence selected from any one of Tables 34, 36 or 37.

In some embodiments, an immune response is elicited in the subject. In some embodiments, the immune response is a humoral response.

In some embodiments, the one or more mutant BTK peptides are administered simultaneously, separately or sequentially.

In some embodiments, the second peptide is sequentially administered after a time period sufficient for the first peptide to activate the second T cells.

In some embodiments, the cancer is selected from the group consisting of certain types of lymphoma and certain types of leukemia. In some embodiments, the cancer is an acute lymphoblastic leukemia (ALL), a mantle cell lymphoma (MCL), a chronic lymphocytic lymphoma or a B-cell non-Hodgkin's lymphoma.

In some embodiments, the further comprising administering at least one additional therapeutic agent or modality.

In some embodiments, the at least one additional therapeutic agent or modality is surgery, a checkpoint inhibitor, an antibody or fragment thereof, a chemotherapeutic agent, radiation, a vaccine, a small molecule, a T cell, a vector, and APC, a polynucleotide, an oncolytic virus or any combination thereof.

In some embodiments, the at least one additional therapeutic agent is an anti-PD-1 agent and anti-PD-L1 agent, an anti-CTLA-4 agent, or an anti-CD40 agent. In some embodiments, the additional therapeutic agent is administered before, simultaneously, or after administering the mutant BTK peptide sequences.

In one aspect, provided herein is a method of treating a cancer in a subject, comprising the steps of: (a) identifying a first protein expressed by the subject, wherein the first protein is encoded by a first HLA allele of the subject and wherein the first HLA allele is an HLA allele provided in any one of one of Tables 34, 37 or 38, (b) administering to the subject (i) a first mutant BTK peptide, wherein the first mutant BTK peptide is a peptide to the first HLA allele provided according any one of one of Tables 34, 36 or 37; or (ii) a polynucleic acid encoding the first mutant BTK peptide.

In one aspect, provided herein is a method of identifying a subject with cancer as a candidate for a therapeutic, the method comprising identifying the subject as a subject that expresses a protein encoded by an HLA of one of Tables 34, 36 or 37, wherein the therapeutic is a mutant BTK peptide or a nucleic acid encoding the mutant BTK peptide, wherein the mutant BTK peptide comprises at least 8 contiguous amino acids of a mutant BTK protein comprising a mutation at C481, wherein the peptide (i) comprises a mutation of C481S, (ii) comprises a sequence of a peptide of any one of Tables 34, 36 or 37 and (iii) binds to a corresponding protein encoded by the HLA of any one of Tables 34, 36 or 37.

In some aspects, provided herein is a composition comprising a polypeptide comprising one or more mutant EGFR peptide sequences from a T790M mutant EGFR protein, the one or more mutant EGFR peptide sequences comprising at least 8 contiguous amino acids selected from the group consisting of:

LIMQLMPF,
TVQLIMQL,
TSTVQLIMQL,
TVQLIMQLM,
VQLIMQLM,
STVQLIMQL,
and
LTSTVQLIM.

In some embodiments, the one or more mutant EGFR peptide sequences are specific for a cognate T cell receptor in complex with an HLA protein.

In some embodiments, the composition comprises a mixture of two or three or more mutant EGFR peptide sequences. In some embodiments, the composition comprises at least 2, 3, 4, 5, 6, 7, 8, 9, or 10 mutant EGFR peptide sequences. In some embodiments at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a mutant EGFR protein.

In some aspects, provided herein is a composition comprising at least one polypeptide comprising one or more mutant EGFR peptide sequences from a T790M mutant EGFR protein, the one or more mutant EGFR peptide sequence comprising at least 8 contiguous amino acids selected from the group consisting of: LIMQLMPF, TVQLIMQL, TSTVQLIMQL, TVQLIMQLM, VQLIMQLM, STVQLIMQL and LTSTVQLIM, further comprising three or more amino acid residues that are heterologous to the mutant EGFR protein, linked to the N-terminus or C-terminus of a mutant EGFR peptide sequence, wherein the three or more amino acid residues enhance processing of the mutant EGFR peptide sequences inside a cell and/or enhance presentation of an epitope of the mutant EGFR peptide sequences.

In some embodiments, the three or more amino acid residues that are heterologous to the mutant EGFR protein comprise an amino acid sequence from CMV-pp65, HIV, MART-1 or a non-viral, non-EGFR endogenous peptide.

In some embodiments, the three or more amino acid residues that are heterologous to the mutant EGFR protein comprise at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

In some embodiments, the three or more amino acid residues that are heterologous to the mutant EGFR protein are linked to the N-terminus or C-terminus of the two or more mutant EGFR peptide sequences comprises at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, or 100 amino acids.

In some embodiments, (Xaa-C terminal)_{C} is any amino acid sequence heterologous to the mutant EGFR protein; and, both N and C are not 0.

In some embodiments, (N-terminal Xaa)_{N} and/or (Xaa-C terminal)_{C} comprises an amino acid sequence of a CMV-pp65, HIV, MART-1 or a non-viral, non-EGFR endogenous protein or peptide.

In some embodiments, N and/or C is an integer greater than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

In some embodiments, N and/or C is an integer less than 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, or 100. In some embodiments, N is 0. In some embodiments, C is 0.

In one aspect, provided herein is a composition comprising a polynucleotide sequence encoding the polypeptide described above. In one embodiment, composition comprises a polynucleotide sequence encoding one or more mutant EGFR peptide sequences disclosed herein.

In some embodiments, the composition comprising one or more mutant EGFR peptide sequences further comprises one or more mutant EGFR peptides selected from the Table 40A-40D.

In some embodiments, the at least one polypeptide comprises at least 3, 4, 5, 6, 7, 8, 9, or 10 mutant EGFR peptide sequences.

In some embodiments, at least one of the mutant EGFR peptide sequences comprises at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a mutant EGFR protein. In some embodiments, at least 2, 3, 4, 5, 6, 7, 8, 9, or 10 of the mutant EGFR peptide sequences comprise at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a mutant EGFR protein. In some embodiments, each of the mutant EGFR peptide sequences or each of the two or more EGFR peptide sequences comprises at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous amino acids of a mutant EGFR protein.

In some embodiments, the at least one polypeptide comprises at least one mutant EGFR peptide sequence that binds to or is predicted to bind to a protein encoded by an HLA allele listed in Table 41 with an affinity of 150 nM or less and/or a half-life of 2 hours or more.

In some embodiments, the mutant EGFR peptide sequences comprise a first mutant EGFR peptide sequence that selected from a group consisting of STVQLIMQL, LIMQLMPF, LTSTVQLIM, TVQLIMQL, TSTVQLIMQL, TVQLIMQLM, and VQLIMQLM and a second mutant EGFR peptide sequence having a T790M mutation.

In some embodiments, the mutant EGFR peptide sequences comprise: (a) a first mutant EGFR peptide sequence that selected from a group consisting of STVQLIMQL, LIMQLMPF, LTSTVQLIM, TVQLIMQL, TSTVQLIMQL, TVQLIMQLM, and VQLIMQLM, wherein the first mutant EGFR peptide sequence binds to or is predicted to bind to a protein encoded by an HLA-A68:02, HLA-C15:02, HLA-A25:01, HLA-B57:03, HLA-C12:02, HLA-C03:02, HLA-A26:01, HLA-C12:03, HLA-C06:02, HLA-C03:03, HLA-B52:01, HLA-A30:01, HLA-C02:02, HLA-C12:03, HLA-A11:01, HLA-A32:01, HLA-A02:04, HLA-A68:01, HLA-B15:09, HLA-C17:01, HLA-C03:04, HLA-B08:01, HLA-A01:01, HLA-B42:01, HLA-B57:01, HLA-B15:01, HLA-B14:02, HLA-B37:01, HLA-A36:01, HLA-C15:02, HLA-B15:09, HLA-C12:02, HLA-B38:01, HLA-C03:03, HLA-A02:03, HLA-B58:02, HLA-C08:01, HLA-B35:01, HLA-B40:01, and/or an HLA-B35:03 allele; and (b) a second EGFR peptide sequence comprising a T790M mutation, wherein the first and the second peptides are not identical.

In some embodiments, the at least one polypeptide comprises at least one mutant EGFR peptide sequence that binds to a protein encoded by an HLA allele with an affinity of less than 10 μM, less than 1 μM, less than 500 nM, less than 400 nM, less than 300 nM, less than 250 nM, less than 200 nM, less than 150 nM, less than 100 nM, or less than 50 nM.

In some embodiments, the at least one polypeptide comprises at least one mutant EGFR peptide sequence that binds to a protein encoded by an HLA allele with a stability of greater than 24 hours, greater than 12 hours, greater than 9 hours, greater than 6 hours, greater than 5 hours, greater than 4 hours, greater than 3 hours, greater than 2 hours, greater than 1 hour, greater than 45 minutes, greater than 30 minutes, greater than 15 minutes, or greater than 10 minutes.

In some embodiments, (N-terminal Xaa)_{N} comprises an amino acid sequence of IDIIMKIRNA, FFFFFFFFFFFFFFFFFFFFIIFFIFFWMC, FFFFFFFFFFFFFFFFFFFFFFFFAAFWFW, IFFIFFIIFFFFFFFFFFFFIIIIIIIWEC, FIFFFIIFFFFFIFFFFFIFIIIIIIFWEC, TEY, WQAGILAR, HSYTTAE, PLTEEKIK, GALHFKPGSR, RRANKDATAE, KAFISHEEKR, TDLSSRFSKS, FDLGGGTFDV, CLLLHYSVSK, or MTEYKLVVV.

In some embodiments, (C-terminal Xaa)_{C} comprises an amino acid sequence of KKNKKDDIKD, AGNDDDDDDDDDDDDDDDDDKKDKDDDDDD, AGNKKKKKKKNNNNNNNNNNNNNNNNN, AGRDDDDDDDDDDDDDDDDDDDDDDDDDDD, GKSALTIQL, GKSALTI, QGQNLKYQ, ILGVLLLI, EKEGKISK, AASDFIFLVT, KELKQVASPF, KKKLINEKKE, KKCDISLQFF, KSTAGDTHLG, ATFYVAVTVP, LTIQLIQNHFVDEYDPTIEDSYRKQVVIDG, or TIQLIQNHFVDEYDPTIEDSYRKQVVIDGE.

In some embodiments, at least one of the mutant EGFR peptide sequences comprises a mutant amino acid not encoded by the genome of a cancer cell of a subject.

In some embodiments, the mutant EGFR peptide sequences are present at a concentration at least 1 μg/mL, at least 10 μg/mL, at least 25 μg/mL, at least 50 μg/mL, or at least 100 μg/mL.

In some embodiments, wherein each of the mutant EGFR peptide sequences are present at a concentration at most 5000 μg/mL, at most 2500 μg/mL, at most 1000 μg/mL, at most 750 μg/mL, at most 500 μg/mL, at most 400 μg/mL, or at most 300 μg/mL.

In some embodiments, each of the mutant EGFR peptide sequences are present at a concentration of from 10 μg/mL to 5000 μg/mL, 10 μg/mL to 4000 μg/mL, 10 μg/mL to 3000 μg/mL, 10 μg/mL to 2000 g/mL, 10 μg/mL to 1000 μg/mL, 25 μg/mL to 500 μg/mL, or 50 μg/mL to 300 μg/mL.

In some embodiments, the composition further comprises an immunomodulatory agent or an adjuvant. In some embodiments, the adjuvant is polyICLC.

In one aspect, provided herein is a pharmaceutical composition comprising: (a) the composition comprising the at least one polypeptide comprises at least one mutant EGFR peptide sequence as described above, and (b) a pharmaceutically acceptable excipient.

In some embodiments, the pharmaceutical composition further comprises a pH modifier.

In some embodiments, the pharmaceutical composition is a vaccine composition.

In some embodiments, the pharmaceutical composition is aqueous.

In some embodiments, the one or more of the at least one polypeptide is bounded by pI>5 and HYDRO>−6, pI>8 and HYDRO>−8, pI<5 and HYDRO>−5, pI>9 and HYDRO<−8, pI>7 and a HYDRO value of >−5.5, pI<4.3 and −4≥HYDRO≥−8, pI>0 and HYDRO<−8, pI>0 and HYDRO>−4, or pI>4.3 and −4≥HYDRO≥−8, pI>0 and HYDRO>−4, or pI>4.3 and HYDRO≤−4, pI>0 and HYDRO>−4, or pI>4.3 and −4≥HYDRO≥−9, 5>pI>12 and −4≥HYDRO≥−9.

In some embodiments, the pharmaceutical composition comprises a pH modifier, which is a base.

In some embodiments, the pH modifier is a conjugate base of a weak acid.

In some embodiments, the pH modifier is a pharmaceutically acceptable salt.

In some embodiments, the pH modifier is a dicarboxylate or tricarboxylate salt.

In some embodiments, the pH modifier is citric acid and/or a citrate salt.

In some embodiments, the citrate salt is disodium citrate and/or trisodium citrate.

In some embodiments, the pH modifier is succinic acid and/or a succinate salt.

In some embodiments, the succinate salt is a disodium succinate and/or a monosodium succinate.

In some embodiments, the succinate salt is disodium succinate hexahydrate.

In some embodiments, the pH modifier is present at a concentration of from 0.1 mM-1 mM.

In some embodiments, the pharmaceutical composition comprises the pharmaceutically acceptable carrier comprises a liquid.

In some embodiments, the pharmaceutically acceptable carrier comprises water.

In some embodiments, the pharmaceutically acceptable carrier comprises a sugar.

In some embodiments, the sugar comprises dextrose.

In some embodiments, the dextrose is present at a concentration of from 1-10% w/v.

In some embodiments, the sugar comprises trehalose.

In some embodiments, the sugar comprises sucrose.

In some embodiments, the pharmaceutically acceptable carrier comprises dimethyl sulfoxide (DMSO).

In some embodiments, the DMSO is present at a concentration from 0.1% to 10%, 0.5% to 5%, or 1% to 3%.

In some embodiments, the pharmaceutically acceptable carrier does not comprise dimethyl sulfoxide (DMSO).

In some embodiments, the pharmaceutical composition is lyophilizable.

In some embodiments, the pharmaceutical composition further comprises an immunomodulator or adjuvant.

In some embodiments, the immunomodulator or adjuvant is selected from the group consisting of poly-ICLC, 1018 ISS, aluminum salts, Amplivax, AS15, BCG, CP-870,893, CpG7909, CyaA, ARNAX, STING agonists, dSLIM, GM-CSF, IC30, IC31, Imiquimod, ImuFact IMP321, IS Patch, ISS, ISCOMATRIX, JuvImmune, LipoVac, MF59, monophosphoryllipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, resiquimod, SRL172, Virosomes and other Virus-like particles, YF-17D, VEGF trap, R848, beta-glucan, Pam3Cys, and Aquila's QS21 stimulon.

In some embodiments, the immunomodulator or adjuvant comprises poly-ICLC. In some embodiments, a ratio of poly-ICLC to peptides in the pharmaceutical composition is from 2:1 to 1:10 v:v. In some embodiments, the ratio of poly-ICLC to peptides in the pharmaceutical composition is about 1:1, 1:2, 1:3, 1:4 or 1:5 v:v. In some embodiments, the ratio of poly-ICLC to peptides in the pharmaceutical composition is about 1:3 v:v.

In one aspect, provided herein is a method of treating a cancer in a subject, comprising administering to the subject the pharmaceutical composition described above.

In one aspect, provided herein is a method of treating cancer in a subject, comprising administering to the subject in need thereof, a composition comprising one or more mutant EGFR peptides, or one or more nucleic acids encoding the one or more mutant EGFR peptides, wherein each mutant EGFR peptide comprises at least 8 contiguous amino acids of a mutant EGFR protein comprising a mutation T790M, wherein the one or more mutant EGFR peptides have an amino acid sequence set forth in Table 40A-40D; wherein at least one of the one or more peptides binds with an affinity of 150 nM or less and/or a half-life of 2 hours or more to a protein encoded by an binds to or is predicted to bind to a protein encoded by an HLA-A68:02, HLA-C15:02, HLA-A25:01, HLA-B57:03, HLA-C12:02, HLA-C03:02, HLA-A26:01, HLA-C12:03, HLA-C06:02, HLA-C03:03, HLA-B52:01, HLA-A30:01, HLA-C02:02, HLA-C12:03, HLA-A11:01, HLA-A32:01, HLA-A02:04, HLA-A68:01, HLA-B15:09, HLA-C17:01, HLA-C03:04, HLA-B08:01, HLA-A01:01, HLA-B42:01, HLA-B57:01, HLA-B15:01, HLA-B14:02, HLA-B37:01, HLA-A36:01, HLA-C15:02, HLA-B15:09, HLA-C12:02, HLA-B38:01, HLA-C03:03, HLA-A02:03, HLA-B58:02, HLA-C08:01, HLA-B35:01, HLA-B40:01, and/or an HLA-B35:03 allele; and, wherein said allele is expressed by the subject.

In one aspect, provided herein is a method of treating a subject with cancer, wherein the method comprises: administering to the subject in need thereof, a polypeptide comprising a mutant EGFR peptide sequence, or a polynucleotide encoding the mutant EGFR peptide, wherein (a) the mutant EGFR peptide has the sequence LIMQLMPF and the subject expresses a protein encoded by an HLA-C03:02 allele, (b) the mutant EGFR peptide has the sequence LTSTVQLIM and the subject expresses a protein encoded by an HLA allele selected from a group consisting of: HLA-C12:03, HLA-C15:02, HLA-B57:01, HLA-B57:01, HLA-A36:01, HLA-C12:02, HLA-C03:03 and HLA-B58:02, (c) the mutant EGFR peptide has the sequence QLIMQLMPF and the subject expresses a protein encoded by an HLA-A26:01 allele, (d) the mutant EGFR peptide has the sequence STVQLIMQL and the subject expresses a protein encoded by an HLA allele selected from a group consisting of: HLA-A68:02, HLA-C15:02, HLA-A25:01, HLA-B57:03, HLA-C12:02, HLA-A26:01, HLA-C12:03, HLA-C06:02, HLA-C03:03, HLA-A30:01, HLA-C02:02, HLA-A11:01, HLA-A32:01, HLA-A02:04, HLA-A68:01, HLA-B15:09, HLA-C03:04, HLA-B38:01, HLA-B57:01, HLA-A02:03, HLA-C08:01, HLA-B35:01 and HLA-B40:01, (e) the mutant EGFR peptide has the sequence STVQLIMQLM and the subject expresses a protein encoded by an HLA-B57:01 allele, (f) the mutant EGFR peptide has the sequence TSTVQLIMQL and the subject expresses a protein encoded by an HLA-C15:02 allele, (g) the mutant EGFR peptide has the sequence TVQLIMQL and the subject expresses a protein encoded by an HLA allele selected from a group consisting of: HLA-C17:01, HLA-B08:01, HLA-B42:01, HLA-B14:02, HLA-B37:01, HLA-B15:09, (h) the mutant EGFR peptide has the sequence TVQLIMQLM and the subject expresses a protein encoded by an HLA-B35:03 allele, or (i) the mutant EGFR peptide has the sequence VQLIMQLM and the subject expresses a protein encoded by an HLA allele selected from a group consisting of HLA-B52:01, HLA-B14:02 and HLA-B37:01.

In some embodiments, the method further comprises administering a second polypeptide composition comprising at least one mutant EGFR peptide, wherein the second mutant EGFR peptide is selected from Table 40A-40D.

In one aspect, provided herein is a method of treating cancer in a subject, the method comprising the steps of (a) identifying a first protein expressed by the subject, wherein the first protein is encoded by a first HLA allele of the subject and wherein the first HLA allele is an HLA allele provided in any one of one of Tables 41 to 43; and (b) administering to the subject (i) a first mutant EGFR peptide, wherein the first mutant EGFR peptide is a peptide to the first HLA allele provided according any one of the Tables 42Ai and ii, 42B or 43, or (ii) a polynucleic acid encoding the first mutant EGFR peptide. In some embodiments, the method of treating a cancer in a subject comprising the steps of: identifying one or more specific HLA subtypes expressed in the subject; administering to the subject, a composition comprising one or more mutant EGFR peptide described herein, such that the one or more peptide binds to at least one HLA subtype expressed by the subject with an affinity of 150 nM or less and/or a half-life of 2 hours or more.

In one aspect, provided herein is a method of treating a cancer in a subject, the method comprises the steps of (a) identifying the subject to express a protein encoded by an HLA-B57:01 allele of the subject's genome; (b) administering to the subject a composition comprising a peptide having a sequence STVQLIMQLM. In one embodiment, the method comprises the steps of (a) identifying if the subject expresses a protein encoded by an HLA-A26:01 allele of the subject's genome; (b) administering to the subject a composition comprising a peptide having a sequence QLIMQLMPF.

In some embodiments, an immune response is elicited in the subject. In one embodiment, the immune response is a humoral response.

In some embodiments, the one or more mutant EGFR peptide sequences are administered simultaneously, separately or sequentially. In some embodiments, the second peptide is sequentially administered after a time period sufficient for the first peptide to activate the second T cells.

In some embodiments, the cancer is selected from the group consisting of is selected from the group consisting of glioblastoma, lung adenocarcinoma, non-small cell lung cancer, lung squamous cell carcinoma, kidney carcinoma, head and neck cancers, ovarian cancers, cervical cancers, bladder cancers, gastric cancers, breast cancers, colorectal cancers, endometrial cancers and esophageal cancers.

In some embodiments, the method further comprises administering at least one additional therapeutic agent or modality.

In some embodiments, the at least one additional therapeutic agent or modality is surgery, a checkpoint inhibitor, an antibody or fragment thereof, a chemotherapeutic agent, radiation, a vaccine, a small molecule, a T cell, a vector, and APC, a polynucleotide, an oncolytic virus or any combination thereof. In some embodiments, the at least one additional therapeutic agent is an anti-PD-1 agent and anti-PD-L1 agent, an anti-CTLA-4 agent, or an anti-CD40 agent. In some embodiments, the additional therapeutic agent is administered before, simultaneously, or after administering the mutant EGFR peptide sequences.

In one aspect, provided herein is a method of identifying a subject with cancer as a candidate for a therapeutic, the method comprising identifying the subject as a subject that expresses a protein encoded by an HLA of one of Tables 41, 42Ai, 42Aii, 42B, or 43, wherein the therapeutic is a mutant EGFR peptide or a nucleic acid encoding the mutant EGFR peptide, wherein the mutant EGFR peptide comprises at least 8 contiguous amino acids of a mutant EGFR protein comprising a mutation at T790, wherein the peptide (i) comprises a mutation of T790M, (ii) comprises a sequence of a peptide of any one of Tables 42Ai, 42Aii, 42B, 43, and 44 and (iii) binds to a corresponding protein encoded by the HLA of any one of Tables 42Ai, 42Aii, 42B, 43, and 44.

In one aspect, provided herein is a method of identifying a subject as a candidate for a therapeutic, the method comprising determining that the subject expresses a protein encoded by an HLA-B57:01 allele, wherein the therapeutic comprises a mutant EGFR peptide having the amino acid sequence STVQLIMQLM.

In one aspect, provided herein is a method of identifying a subject as a candidate for a therapeutic, the method comprising determining that the subject expresses a protein encoded by an HLA-A26:01 allele, wherein the therapeutic comprises a mutant EGFR peptide having the amino acid sequence QLIMQLMPF.

### BRIEF DESCRIPTION OF THE DRAWINGS

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIG. 1 illustrates an exemplary workflow for determination of GATA3 epitopes that can induce CD8+ and/or CD4+ T cells.

FIG. 2 illustrates an exemplary workflow of experiments for determining whether epitopes are processed and presented (top) and whether epitopes are recognized by T cells (bottom). This workflow confirmed that GATA3 neoantigens were processed and presented (detected by mass spectrometry) and GATA3 neoantigens bound to an HLA multimer could be recognized by a recombinant T cell receptor (TCR) expressed in a Jurkat cell line.

FIG. 3 illustrates an exemplary schematic of a workflow for detection of GATA3 neoORF epitopes by mass spectrometry. For peptide isolation, batch lysis was performed and an HLA class I pan antibody (W6/32) is used for immunoprecipitation.

FIG. 4 illustrates an exemplary schematic of a workflow for GATA3 antigen-specific expansion of CD8+ T cells.

FIG. 5 illustrates a summary of experiments showing that predicted GATA3 epitopes to HLA-A02 (left), HLA-B07 (middle) and HLA-B08 (right) can be detected by mass spectrometry.

FIG. 6 is an illustration of the GATA3 neoORF. The shaded region represents the portion of the GATA3 neoORF sequence portion shared by all patients (common region) and shared by some patients (variable region).

FIG. 7A is an illustration of the GATA3 neoORF sequence (SEQ ID NO: 2) with the variable region sequence (SEQ ID NO: 3) and common region sequences (SEQ ID NO: 4).

FIG. 7B depicts a schematic showing the GATA3 sequence (SEQ ID NO: 1) with the neoORF sequence (SEQ ID NO: 2) and that 3 predicted HLA-02:01 epitopes, 2 predicted HLA-B07:02 epitopes and 1 predicted HLA-B08:01 epitopes were observed by mass spectrometry. This data shows that the epitopes are targetable.

FIG. 7C is an illustration of an example of a peptide design scheme of overlapping peptides (OLPs) across the entire GATA3 neoORF region.

FIG. 7D is an exemplary amino acid sequence of variable region of GATA3 neo ORF (SEQ ID NO: 3)

FIG. 7E is an exemplary amino acid sequence of common region of GATA3 neo ORF (SEQ ID NO: 4)

FIG. 8 is a graph depicting the number of therapeutic class I GATA3 neoORF epitopes vs percent of patients containing these epitopes. Most patients will have 4-5 epitopes.

FIG. 9A depicts example results showing antigen specific CD8⁺ T cell responses to the indicated peptide using a PBMC sample from a human donor.

FIG. 9B depicts example results showing antigen specific CD8⁺ T cell responses to the indicated peptides using PBMC samples from human donors.

FIG. 9C depicts example results showing antigen specific CD8⁺ T cell responses to the indicated peptides using PBMC samples from human donors.

FIG. 10A depicts example results showing antigen specific CD8⁺ T cell responses to the indicated peptides using PBMC samples from human donors.

FIG. 10B depicts example results showing antigen specific CD8⁺ T cell responses to the indicated peptides using PBMC samples from human donors.

FIG. 11 depicts a FACS analysis of antigen-specific induction of IFNγ and TNFα levels of CD4⁺ cells from a healthy HLA-A02:01 donor stimulated with APCs loaded with or without a GATA3 neoORF peptide.

FIG. 12A shows that the indicated peptides were soluble at the indicated peptide concentrations in the pharmaceutical compositions with a 5 mM or 0.25 mM succinate, no DMSO, 5% dextrose in water (5DW) and no polyICLC.

FIG. 12B shows that the indicated peptides were soluble at the indicated peptide concentrations in the pharmaceutical compositions with a 5 mM or 0.25 mM succinate, no DMSO, 5% dextrose in water (5DW) and with polyICLC.

FIG. 12C shows that the indicated peptides were soluble at the indicated peptide concentrations in the pharmaceutical compositions with a 5 mM or 0.25 mM succinate, no DMSO, 5% dextrose in water (5DW) and with polyICLC at the indicated peptide:polyICLC ratio.

FIG. 13 shows amino acid sequence of the common region of GATA3 frame-shift mutations (SEQ ID NO: 4).

FIG. 14 shows Kaplan-Meier survival curve for patients in the MSK-IMPACT breast cancer dataset.

FIG. 15 shows simulated count of presented epitopes per patient.

FIG. 16 shows alignment of GATA3 wild-type and mutation nucleotide sequences.

FIG. 17 shows alignment of GATA3 wild-type and mutation amino acid sequences.

FIG. 18 shows GATA3 mutation encoded plasmid map.

FIG. 19 shows multi-alignment of GATA3 mutation gene and DNA sequencing data of GATA3 mutation plasmid construct.

FIG. 20 shows the restriction enzyme digestion of GATA3 mutation plasmid with AfIII.

FIG. 21 shows MHC class I and MHC class II expression of the GATA3 transduced HEK 293T cells.

FIGS. 22A-22D show HLA-A02 and MHC-ABC expression profile of HLA-A02.01, HLA-B07.02, and HLA-B08.01 transfected GATA3 HEK293T cells.

FIG. 22A shows Non-transfected GATA3 HEK293T cells.

FIG. 22B shows HLA-A02.01 transfected GATA3 HEK293T cells.

FIG. 22C shows HLA-B07.02 transfected GATA3 HEK293T cells.

FIG. 22D shows HLA-B08.01 transfected GATA3 HEK293T cells.

FIG. 23 shows detection of predicted peptide epitopes derived from the common region of the GATA3 neoORF stably expressed in HEK293T cells. The sequence in light gray and black indicate the variable and common regions of the GATA3 neoORF, respectively.

FIG. 24A shows MS/MS spectra for the endogenously processed peptide epitope SMLTGPPARV (bottom) and its corresponding synthetic peptide (top).

FIG. 24B shows head-to-toe plot of MS/MS spectral match.

FIG. 25A shows MS/MS spectra for the endogenously processed peptide epitope MLTGPPARV (bottom) and its corresponding synthetic peptide (top).

FIG. 25B shows Head-to-toe plot of spectral match.

FIG. 26A shows MS/MS spectra for the endogenously processed peptide epitope KPKRDGYMF (bottom) and its corresponding synthetic peptide (top).

FIG. 26B shows Head-to-toe plot of spectral match.

FIG. 27A shows MS/MS spectra for the endogenously processed peptide epitope KPKRDGYMFL (bottom) and its corresponding synthetic peptide (top).

FIG. 27B shows Head-to-toe plot of spectral match.

FIG. 28A shows MS/MS spectra for the endogenously processed peptide epitope ESKImFATL (bottom) and its corresponding synthetic peptide (top).

FIG. 28B shows Head-to-toe plot of spectral match.

FIG. 29A shows representative induction of CD8+ responses with GATA3 neoORF specific peptide (FLT-mDC GATA3 Stim2 Multimer).

FIG. 29 B shows negative control with no induction of CD8+ responses in PBMC and dendritic cells.

FIG. 30A shows induction of antigen specific CD4 T cells with no peptide.

FIG. 30B shows induction of antigen specific CD4 T cells with GATA3 neoORF specific peptide.

FIGS. 31A-31D show GATA3 specific CD8+ T cells by multimer staining.

FIG. 31A shows GATA3 specific CD8+ T cells were observed at average of 1.16% positive after long term stimulation for healthy donor HD47.

FIG. 31B shows GATA3 specific CD8+ T cells were observed at average of 1.29%, positive after long term stimulation for healthy donor HD50.

FIG. 31C shows GATA3 specific CD8+ T cells were observed at average of 1.9% positive after long term stimulation for healthy donor HD51.

FIG. 31D shows GATA3 specific CD8+ T cells were observed at average of 4.5% positive after long term stimulation for healthy donor HD51 at a different concentration of peptide than in FIG. 31C.

FIG. 32 shows comparison of Caspase-3 positive fraction of live target cells. 4 different GATA3 induced healthy donor PBMC 1 to 4 were co-cultured with GATA3 mutation transduced HEK 293T cells (GATA3Trd) or non-transduced HEK 293T cells (NoTRd293T) as negative control group.

FIG. 33 shows significant difference between GATA3 transduced HEK293T cells and non-transduced HEK293T cells.

FIG. 34 shows CD107a expression difference of CD8+ T cells co-culture with GATA3 transduced HEK293T cells or non-transduced HEK293T cells.

FIG. 35 shows IFN-γ concentration difference in co-culture condition between GATA3 transduced HEK293T cells and non-transduced HEK293T cells with GATA3 induced T cells.

FIG. 36 shows overview of GATA3 specific TCR cloning. The details are described in Example 26.

FIG. 37 shows exemplary methods for generating GATA3 specific TCR transduced Jurkat and PBMC. The details are described in Example 26.

FIG. 38 shows overview of functional assay with TCR transduced Jurkat.

FIG. 39 shows GATA3 specific CD8+ T cell by multimer staining for sorting.

FIG. 40 shows GATA3 specific TCR construct for lenti-virus.

FIG. 41A shows multi-alignment of GATA3 TCR alpha sequence and wild type DNA sequence.

FIG. 41B shows multi-alignment of GATA3 TCR beta sequence and wild type DNA sequence.

FIG. 42 shows restriction enzyme digestion of GATA3 TCR plasmid with AfIII.

FIG. 43 shows GATA3 specific TCR transduced Jurkat stained with GATA3 multimer PE and GATA3 multimer BV650.

FIG. 44 shows GATA3 specific TCR peptide titration assay.

FIG. 45 shows IL-2 release assay of GATA3 specific TCR transduced Jurkat cells and GATA3 mutation transduced target cells.

FIG. 46 shows stereochemistry of exemplary GATA3 neo ORF peptide. The peptide consists of 14 amino acids and sequence of ESKIMFATLQRSSL. The peptide has a molecular formula of C₇₀H₁₁₉N₁₉O₂₂S and molecular weight of 1610.89 g/mol. The peptide is in the trifluoroacetic acid (TFA) salt form.

FIG. 47 shows stereochemistry of exemplary GATA3 neo ORF peptide. The peptide consists of 16 amino acids and sequence of KPKRDGYMFLKAESKI. The peptide has a molecular formula of C₈₇H₁₄₃N₂₃O₂₃S and molecular weight of 1911.30 g/mol. The peptide is in the trifluoroacetic acid (TFA) salt form.

FIG. 48 shows stereochemistry of exemplary GATA3 neo ORF peptide. The peptide consists of 18 amino acids and sequence of SMLTGPPARVPAVPFDLH. The peptide has a molecular formula of C₈₇H₁₃₇N₂₃O₂₃S and molecular weight of 1905.25 g/mol. The peptide is in the trifluoroacetic acid (TFA) salt form.

FIG. 49 shows stereochemistry of exemplary GATA3 neo ORF peptide. The peptide consists of 21 amino acids and sequence of EPCSMLTGPPARVPAVPFDLH. The peptide has a molecular formula of C₁₀₀H₁₅₆N₂₆O₂₈S₂ and molecular weight of 2234.62 g/mol. The peptide is in the trifluoroacetic acid (TFA) salt form.

FIG. 50 shows stereochemistry of exemplary GATA3 neo ORF peptide. The peptide consists of 25 amino acids and sequence of LHFCRSSIMKPKRDGYMFLKAESKI. The peptide has a molecular formula of C₁₃₄H₂₁₇N₃₇O₃₄S₃ and molecular weight of 2986.62 g/mol. The peptide is in the trifluoroacetic acid (TFA) salt form.

FIG. 51 shows stereochemistry of exemplary GATA3 neo ORF peptide. The peptide consists of 26 amino acids and sequence of GPPARVPAVPFDLHFCRSSIMKPKRD. The peptide has a molecular formula of C₁₃₁H₂₀₉N₃₉O₃₃S₂ and molecular weight of 2922.47 g/mol. The peptide is in the trifluoroacetic acid (TFA) salt form.

FIG. 52 shows stereochemistry of exemplary GATA3 neo ORF peptide. The peptide consists of 33 amino acids and sequence of KPKRDGYMFLKAESKIMFATLQRSSLWCLCSNH. The peptide has a molecular formula of C₁₇₃H₂₇₄N₄₈O₄₆S₄ and molecular weight of 3890.63 g/mol. The peptide is in the trifluoroacetic acid (TFA) salt form.

FIG. 53 shows BTK antigen peptide specific CD8⁺ T cell responses using PBMC samples from human donors.

FIG. 54 shows EGFR antigen peptide specific CD8⁺ T cell responses using PBMC samples from human donors.

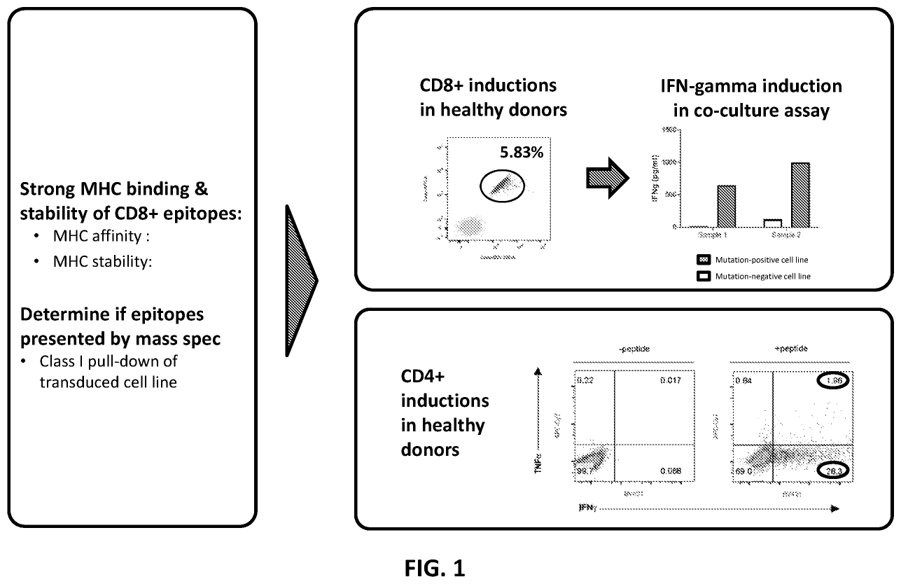

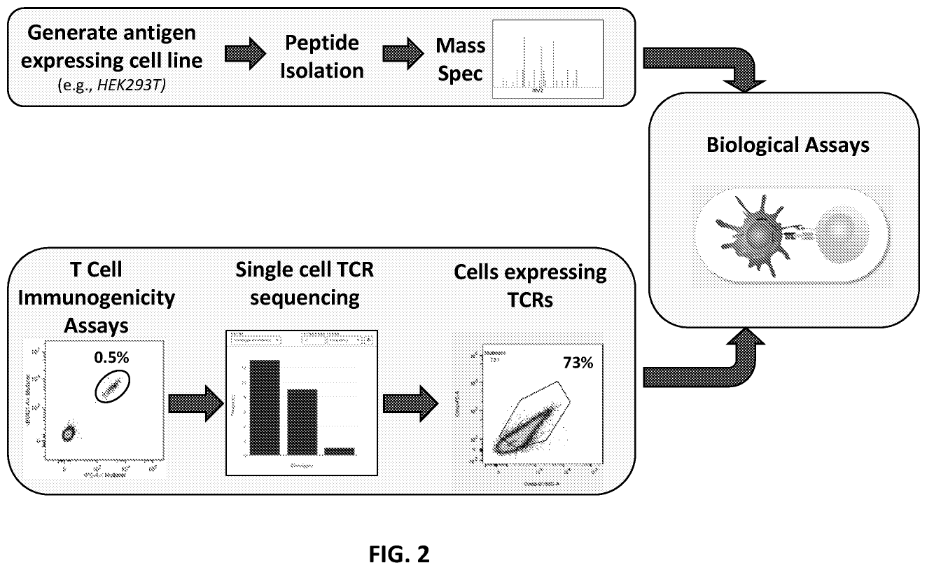

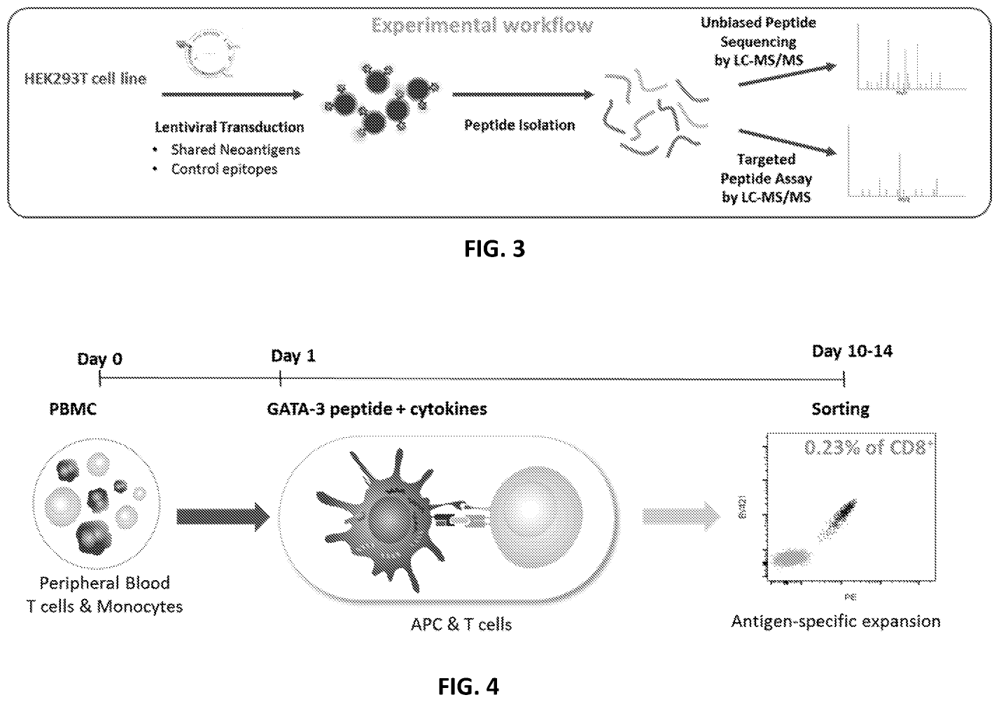

### DETAILED DESCRIPTION

GATA3 is a gene that is highly expressed in breast cancer, and is one of the most frequently mutated genes in these cancers. The most common classes of mutations in this gene are insertions or deletions between nucleotides encoding amino acids 393 and 445 (the natural stop codon). When these shift the open reading frame to the +1 frame, they result in an extended novel reading frame (“neoORF”) that leads at least 61 and as many as 113 amino acids that are not normally expressed in healthy cells. The 61 amino acids are shared between all patients (conserved region), while each patient will have 0-52 additional amino acids (variable region). Epitopes that are processed and presented from this neoORF are therefore neoantigens that are shared between some or all patients that harbor this same class of mutations. The GATA3 neoORF appears to be an adverse prognostic factor in breast cancer. GATA3 wild-type is a highly expressed gene and the GATA3 neoORF retains high expression. The GATA3 neoORF is translated and is associated with increased risk of breast cancer.

In some embodiments, overlapping long peptides (OLPs) that cover the entire neoORF can be used for treating cancer. In some aspects, the OLPs described herein have been designed to include epitopes on the ends of peptides that simplify the process of processing and presentation (as only one cleavage event is necessary). In some aspects, short peptides (e.g., 9-11 amino acids) can be administered to a subject to treat cancer that bind to an MHC class I protein. The approaches described herein can be used to target many neoantigens without needing to select patients based on their HLA composition.

In some embodiments, peptides described herein can comprise a modification that may increase immunogenicity (e.g., lipidation). In some embodiments, a polynucleotide encoding a polypeptide encoded by the entire GATA3 neoORF (e.g., polybodies) is provided. In some embodiments, a cell-based therapy, such as engineered T cells expressing TCRs targeting specific epitopes can be used to treat a subject with cancer.

Synthetic long peptides (SLPs) that cover the common region of GATA3 protein are disclosed herein. These peptides are soluble in the formulations described herein and compatible with polyICLC for s.c. injections. High purities and synthesis yields of one or more of these peptides can be achieved by adopting pseudo-proline building blocks during the solid phase peptide synthesis (SPPS). Purification conditions of each of these peptides have been developed as well.

Described herein are new immunotherapeutic agents and uses thereof based on the discovery of neoantigens arising from mutational events unique to an individual's tumor. Accordingly, the present disclosure described herein provides peptides, polynucleotides encoding the peptides, and peptide binding agents that can be used, for example, to stimulate an immune response to a tumor associated antigen or neoepitope, to create an immunogenic composition or cancer vaccine for use in treating disease.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### Definitions

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

“Major Histocompatibility Complex” or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, Fundamental Immunology, 3^{rd} Ed., Raven Press, New York (1993). “Proteins or molecules of the major histocompatibility complex (MHC)”, “MHC molecules”, “MHC proteins” or “HLA proteins” are to be understood as meaning proteins capable of binding peptides resulting from the proteolytic cleavage of protein antigens and representing potential lymphocyte epitopes, (e.g., T cell epitope and B cell epitope) transporting them to the cell surface and presenting them there to specific cells, in particular cytotoxic T-lymphocytes, T-helper cells, or B cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The cellular biology and the expression patterns of the two MHC classes are adapted to these different roles.

“Human Leukocyte Antigen” or “HLA” is a human class I or class II Major Histocompatibility Complex (MHC) protein (see, e.g., Stites, et al., Immunology, 8^{th} Ed., Lange Publishing, Los Altos, Calif. (1994).

“Polypeptide”, “peptide” and their grammatical equivalents as used herein refer to a polymer of amino acid residues, typically L-amino acids, connected one to the other, typically by peptide bonds between the α-amino and carboxyl groups of adjacent amino acids. Polypeptides and peptides include, but are not limited to, “mutant peptides”, “neoantigen peptides” and “neoantigenic peptides”. Polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. A “mature protein” is a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, 0-phenylserine O-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitination, addition of pyrrolidone carboxylic acid, formation of disulfide bridges, sulfation, myristoylation, palmitoylation, isoprenylation, farnesylation, geranylation, glypiation, lipoylation and iodination.

A peptide or polypeptide may comprise at least one flanking sequence. The term “flanking sequence” as used herein refers to a fragment or region of a peptide that is not a part of an epitope.

An “immunogenic” peptide or an “immunogenic” epitope or “peptide epitope” is a peptide that comprises an allele-specific motif such that the peptide will bind an HLA molecule and induce a cell-mediated or humoral response, for example, cytotoxic T lymphocyte (CTL (e.g., CD8⁺)), helper T lymphocyte (Th (e.g., CD4⁺)) and/or B lymphocyte response. Thus, immunogenic peptides described herein are capable of binding to an appropriate HLA molecule and thereafter inducing a CTL (cytotoxic) response, or a HTL (and humoral) response, to the peptide.

“Neoantigen” means a class of tumor antigens which arise from tumor-specific changes in proteins. Neoantigens encompass, but are not limited to, tumor antigens which arise from, for example, substitution in the protein sequence, frame shift mutation, fusion polypeptide, in-frame deletion, insertion, expression of endogenous retroviral polypeptides, and tumor-specific overexpression of polypeptides.

The term “residue” refers to an amino acid residue or amino acid mimetic residue incorporated into a peptide or protein by an amide bond or amide bond mimetic, or nucleic acid (DNA or RNA) that encodes the amino acid or amino acid mimetic.

A “neoepitope”, “tumor specific neoepitope” or “tumor antigen” refers to an epitope or antigenic determinant region that is not present in a reference, such as a non-diseased cell, e.g., a non-cancerous cell or a germline cell, but is found in a diseased cell, e.g., a cancer cell. This includes situations where a corresponding epitope is found in a normal non-diseased cell or a germline cell but, due to one or more mutations in a diseased cell, e.g., a cancer cell, the sequence of the epitope is changed so as to result in the neoepitope. The term “neoepitope” as used herein refers to an antigenic determinant region within the peptide or neoantigenic peptide. A neoepitope may comprise at least one “anchor residue” and at least one “anchor residue flanking region.” A neoepitope may further comprise a “separation region.” The term “anchor residue” refers to an amino acid residue that binds to specific pockets on HLAs, resulting in specificity of interactions with HLAs. In some cases, an anchor residue may be at a canonical anchor position. In other cases, an anchor residue may be at a non-canonical anchor position. Neoepitopes may bind to HLA molecules through primary and secondary anchor residues protruding into the pockets in the peptide-binding grooves. In the peptide-binding grooves, specific amino acids compose pockets that accommodate the corresponding side chains of the anchor residues of the presented neoepitopes. Peptide-binding preferences exist among different alleles of both of HLA I and HLA II molecules. HLA class I molecules bind short neoepitopes, whose N- and C-terminal ends are anchored into the pockets located at the ends of the neoepitope binding groove. While the majority of the HLA class I binding neoepitopes are of about 9 amino acids, longer neoepitopes can be accommodated by the bulging of their central portion, resulting in binding neoepitopes of about 8 to 12 amino acids. Neoepitopes binding to HLA class II proteins are not constrained in size and can vary from about 16 to 25 amino acids. The neoepitope binding groove in the HLA class II molecules is open at both ends, which enables binding of peptides with relatively longer length. Though the core 9 amino acid residues long segment contributes the most to the recognition of the neoepitope, the anchor residue flanking regions are also important for the specificity of the peptide to the HLA class II allele. In some cases, the anchor residue flanking region is N-terminus residues. In another case, the anchor residue flanking region is C-terminus residues. In yet another case, the anchor residue flanking region is both N-terminus residues and C-terminus residues. In some cases, the anchor residue flanking region is flanked by at least two anchor residues. An anchor residue flanking region flanked by anchor residues is a “separation region.”

A “reference” can be used to correlate and compare the results obtained in the methods of the present disclosure from a tumor specimen. Typically the “reference” may be obtained on the basis of one or more normal specimens, in particular specimens which are not affected by a cancer disease, either obtained from a patient or one or more different individuals, for example, healthy individuals, in particular individuals of the same species. A “reference” can be determined empirically by testing a sufficiently large number of normal specimens.

An “epitope” is the collective features of a molecule, such as primary, secondary and tertiary peptide structure, and charge, that together form a site recognized by, for example, an immunoglobulin, T cell receptor, HLA molecule, or chimeric antigen receptor. Alternatively, an epitope can be defined as a set of amino acid residues which is involved in recognition by a particular immunoglobulin, or in the context of T cells, those residues necessary for recognition by T cell receptor proteins, chimeric antigen receptors, and/or Major Histocompatibility Complex (MHC) receptors. A “T cell epitope” is to be understood as meaning a peptide sequence which can be bound by the MHC molecules of class I or II in the form of a peptide-presenting MHC molecule or MHC complex and then, in this form, be recognized and bound by T cells, such as T-lymphocytes or T-helper cells. Epitopes can be prepared by isolation from a natural source, or they can be synthesized according to standard protocols in the art. Synthetic epitopes can comprise artificial amino acid residues, “amino acid mimetics,” such as D isomers of naturally-occurring L amino acid residues or non-naturally-occurring amino acid residues such as cyclohexylalanine. Throughout this disclosure, epitopes may be referred to in some cases as peptides or peptide epitopes. It is to be appreciated that proteins or peptides that comprise an epitope or an analog described herein as well as additional amino acid(s) are still within the bounds of the present disclosure. In certain embodiments, the peptide comprises a fragment of an antigen. In certain embodiments, there is a limitation on the length of a peptide of the present disclosure. The embodiment that is length-limited occurs when the protein or peptide comprising an epitope described herein comprises a region (i.e., a contiguous series of amino acid residues) having 100% identity with a native sequence. In order to avoid the definition of epitope from reading, e.g., on whole natural molecules, there is a limitation on the length of any region that has 100% identity with a native peptide sequence. Thus, for a peptide comprising an epitope described herein and a region with 100% identity with a native peptide sequence, the region with 100% identity to a native sequence generally has a length of: less than or equal to 600 amino acid residues, less than or equal to 500 amino acid residues, less than or equal to 400 amino acid residues, less than or equal to 250 amino acid residues, less than or equal to 100 amino acid residues, less than or equal to 85 amino acid residues, less than or equal to 75 amino acid residues, less than or equal to 65 amino acid residues, and less than or equal to 50 amino acid residues. In certain embodiments, an “epitope” described herein is comprised by a peptide having a region with less than 51 amino acid residues that has 100% identity to a native peptide sequence, in any increment down to 5 amino acid residues; for example 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 amino acid residues.

The nomenclature used to describe peptides or proteins follows the conventional practice wherein the amino group is presented to the left (the amino- or N-terminus) and the carboxyl group to the right (the carboxy- or C-terminus) of each amino acid residue. When amino acid residue positions are referred to in a peptide epitope they are numbered in an amino to carboxyl direction with position one being the residue located at the amino terminal end of the epitope, or the peptide or protein of which it can be a part. In the formula representing selected specific embodiments of the present disclosure, the amino- and carboxyl-terminal groups, although not specifically shown, are in the form they would assume at physiologic pH values, unless otherwise specified. In the amino acid structure formula, each residue is generally represented by standard three letter or single letter designations. The L-form of an amino acid residue is represented by a capital single letter or a capital first letter of a three-letter symbol, and the D-form for those amino acid residues having D-forms is represented by a lower case single letter or a lower case three letter symbol. However, when three letter symbols or full names are used without capitals, they can refer to L amino acid residues. Glycine has no asymmetric carbon atom and is simply referred to as “Gly” or “G”. The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine.)

The term “mutation” refers to a change of or difference in the nucleic acid sequence (nucleotide substitution, addition or deletion) compared to a reference. A “somatic mutation” can occur in any of the cells of the body except the germ cells (sperm and egg) and therefore are not passed on to children. These alterations can (but do not always) cause cancer or other diseases. In some embodiments, a mutation is a non-synonymous mutation. The term “non-synonymous mutation” refers to a mutation, for example, a nucleotide substitution, which does result in an amino acid change such as an amino acid substitution in the translation product. A “frameshift” occurs when a mutation disrupts the normal phase of a gene's codon periodicity (also known as “reading frame”), resulting in the translation of a non-native protein sequence. It is possible for different mutations in a gene to achieve the same altered reading frame.

A “conservative” amino acid substitution is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate peptide function are well-known in the art.

As used herein, the term “affinity” refers to a measure of the strength of binding between two members of a binding pair, for example, an HLA-binding peptide and a class I or II HLA. K_{D} is the dissociation constant and has units of molarity. The affinity constant is the inverse of the dissociation constant. An affinity constant is sometimes used as a generic term to describe this chemical entity. It is a direct measure of the energy of binding. Affinity may be determined experimentally, for example by surface plasmon resonance (SPR) using commercially available Biacore SPR units. Affinity may also be expressed as the inhibitory concentration 50 (IC₅₀), that concentration at which 50% of the peptide is displaced. Likewise, ln(IC₅₀) refers to the natural log of the IC₅₀. K_{off} refers to the off-rate constant, for example, for dissociation of an HLA-binding peptide and a class I or II HLA. Throughout this disclosure, “binding data” results can be expressed in terms of “IC₅₀.” IC₅₀ is the concentration of the tested peptide in a binding assay at which 50% inhibition of binding of a labeled reference peptide is observed. Given the conditions in which the assays are run (i.e., limiting HLA protein and labeled reference peptide concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC₅₀, relative to the IC₅₀ of a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)). “Cross-reactive binding” indicates that a peptide is bound by more than one HLA molecule; a synonym is degenerate binding.

The term “derived” and its grammatical equivalents when used to discuss an epitope is a synonym for “prepared” and its grammatical equivalents. A derived epitope can be isolated from a natural source, or it can be synthesized according to standard protocols in the art. Synthetic epitopes can comprise artificial amino acid residues “amino acid mimetics,” such as D isomers of natural occurring L amino acid residues or non-natural amino acid residues such as cyclohexylalanine. A derived or prepared epitope can be an analog of a native epitope.

A “native” or a “wild type” sequence refers to a sequence found in nature. Such a sequence can comprise a longer sequence in nature.

A “receptor” is to be understood as meaning a biological molecule or a molecule grouping capable of binding a ligand. A receptor may serve, to transmit information in a cell, a cell formation or an organism. The receptor comprises at least one receptor unit, for example, where each receptor unit may consist of a protein molecule. The receptor has a structure which complements that of a ligand and may complex the ligand as a binding partner. The information is transmitted in particular by conformational changes of the receptor following complexation of the ligand on the surface of a cell. In some embodiments, a receptor is to be understood as meaning in particular proteins of MHC classes I and II capable of forming a receptor/ligand complex with a ligand, in particular a peptide or peptide fragment of suitable length.

A “ligand” is to be understood as meaning a molecule which has a structure complementary to that of a receptor and is capable of forming a complex with this receptor. In some embodiments, a ligand is to be understood as meaning a peptide or peptide fragment which has a suitable length and suitable binding motifs in its amino acid sequence, so that the peptide or peptide fragment is capable of forming a complex with proteins of MHC class I or MHC class II.

In some embodiments, a “receptor/ligand complex” is also to be understood as meaning a “receptor/peptide complex” or “receptor/peptide fragment complex”, including a peptide- or peptide fragment-presenting MHC molecule of class I or of class II.

“Synthetic peptide” refers to a peptide that is obtained from a non-natural source, e.g., is man-made. Such peptides can be produced using such methods as chemical synthesis or recombinant DNA technology. “Synthetic peptides” include “fusion proteins”.

The term “motif” refers to a pattern of residues in an amino acid sequence of defined length, for example, a peptide of less than about 15 amino acid residues in length, or less than about 13 amino acid residues in length, for example, from about 8 to about 13 amino acid residues (e.g., 8, 9, 10, 11, 12, or 13) for a class I HLA motif and from about 6 to about 25 amino acid residues (e.g., 6, 7, 8, 9, 10, 11, 12, 13, 14, 15, 16, 17, 18, 19, 20, 21, 22, 23, 24, or 25) for a class II HLA motif, which is recognized by a particular HLA molecule. Motifs are typically different for each HLA protein encoded by a given human HLA allele. These motifs differ in their pattern of the primary and secondary anchor residues. In some embodiments, an MHC class I motif identifies a peptide of 9, 10, or 11 amino acid residues in length.

The term “naturally occurring” and its grammatical equivalents as used herein refer to the fact that an object can be found in nature. For example, a peptide or nucleic acid that is present in an organism (including viruses) and can be isolated from a source in nature and which has not been intentionally modified by man in the laboratory is naturally occurring.

According to the present disclosure, the term “vaccine” relates to a pharmaceutical preparation (pharmaceutical composition) or product that upon administration induces an immune response, for example, a cellular or humoral immune response, which recognizes and attacks a pathogen or a diseased cell such as a cancer cell. A vaccine may be used for the prevention or treatment of a disease. The term “individualized cancer vaccine” or “personalized cancer vaccine” concerns a particular cancer patient and means that a cancer vaccine is adapted to the needs or special circumstances of an individual cancer patient.

“Antigen processing” or “processing” and its grammatical equivalents refers to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, for example, antigen presenting cells, to specific T cells.

“Antigen presenting cells” (APC) are cells which present peptide fragments of protein antigens in association with MHC molecules on their cell surface. Some APCs may activate antigen specific T cells. Professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen presenting cell. An additional co-stimulatory signal is then produced by the antigen presenting cell, leading to activation of the T cell. The expression of co-stimulatory molecules is a defining feature of professional antigen-presenting cells. The main types of professional antigen-presenting cells are dendritic cells, which have the broadest range of antigen presentation, and are probably the most important antigen presenting cells, macrophages, B-cells, and certain activated epithelial cells. Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes. However, this nomenclature should not be construed to exclude all possible intermediate stages of differentiation. Immature dendritic cells are characterized as antigen presenting cells with a high capacity for antigen uptake and processing, which correlates with the high expression of Fc receptor (FcR) and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1 BB).

The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci. U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can have 50%, 60%, 70%, 75%, 80%, 85%, 90%, 98%, 99% or 100% sequence identity to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure. In some embodiments, an “isolated polynucleotide” encompasses a PCR or quantitative PCR reaction comprising the polynucleotide amplified in the PCR or quantitative PCR reaction.

The term “isolated”, “biologically pure” or their grammatical equivalents refers to material which is substantially or essentially free from components which normally accompany the material as it is found in its native state. Thus, isolated peptides described herein do not contain some or all of the materials normally associated with the peptides in their in situ environment. An “isolated” epitope refers to an epitope that does not include the whole sequence of the antigen from which the epitope was derived. Typically the “isolated” epitope does not have attached thereto additional amino acid residues that result in a sequence that has 100% identity over the entire length of a native sequence. The native sequence can be a sequence such as a tumor-associated antigen from which the epitope is derived. Thus, the term “isolated” means that the material is removed from its original environment (e.g., the natural environment if it is naturally occurring). An “isolated” nucleic acid is a nucleic acid removed from its natural environment. For example, a naturally-occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. Isolated RNA molecules include in vivo or in vitro RNA transcripts of the DNA molecules described herein, and further include such molecules produced synthetically.

The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

The term “substantially pure” as used herein refers to material which is at least 50% pure (i.e., free from contaminants), at least 90% pure, at least 95% pure, at least 98% pure, or at least 99% pure.

The terms “polynucleotide”, “nucleotide”, “nucleic acid”, “polynucleic acid” or “oligonucleotide” and their grammatical equivalents are used interchangeably herein and refer to polymers of nucleotides of any length, and include DNA and RNA, for example, mRNA. Thus, these terms include double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein may be introduced into a cell by, for example, transfection, transformation, or transduction. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered using the methods of the present disclosure is mRNA.

“Transfection,” “transformation,” or “transduction” as used herein refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder; and (2) prophylactic or preventative measures that prevent or slow the development of a targeted pathologic condition or disorder. Thus those in need of treatment include those already with the disorder; those prone to have the disorder; and those in whom the disorder is to be prevented.

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

### Neoantigens and Uses Thereof

One of the critical barriers to developing curative and tumor-specific immunotherapy is the identification and selection of highly specific and restricted tumor antigens to avoid autoimmunity. Tumor neoantigens, which arise as a result of genetic change (e.g., inversions, translocations, deletions, missense mutations, splice site mutations, etc.) within malignant cells, represent the most tumor-specific class of antigens. Neoantigens have rarely been used in cancer vaccine or immunogenic compositions due to technical difficulties in identifying them, selecting optimized antigens, and producing neoantigens for use in a vaccine or immunogenic composition. These problems may be addressed by: identifying mutations in neoplasias/tumors which are present at the DNA level in tumor but not in matched germline samples from a high proportion of subjects having cancer; analyzing the identified mutations with one or more peptide-MHC binding prediction algorithms to generate a plurality of neoantigen T cell epitopes that are expressed within the neoplasia/tumor and that bind to a high proportion of patient HLA alleles; and synthesizing the plurality of neoantigenic peptides selected from the sets of all neoantigen peptides and predicted binding peptides for use in a cancer vaccine or immunogenic composition suitable for treating a high proportion of subjects having cancer.

For example, translating peptide sequencing information into a therapeutic vaccine may include prediction of mutated peptides that can bind to HLA molecules of a high proportion of individuals. Efficiently choosing which particular mutations to utilize as immunogen requires the ability to predict which mutated peptides would efficiently bind to a high proportion of patient's HLA alleles. Recently, neural network based learning approaches with validated binding and non-binding peptides have advanced the accuracy of prediction algorithms for the major HLA-A and -B alleles. However, even using advanced neural network-based algorithms to encode HLA-peptide binding rules, several factors limit the power to predict peptides presented on HLA alleles.

Another example of translating peptide sequencing information into a therapeutic vaccine may include formulating the drug as a multi-epitope vaccine of long peptides. Targeting as many mutated epitopes as practically as possible takes advantage of the enormous capacity of the immune system, prevents the opportunity for immunological escape by down-modulation of an immune targeted gene product, and compensates for the known inaccuracy of epitope prediction approaches. Synthetic peptides provide a useful means to prepare multiple immunogens efficiently and to rapidly translate identification of mutant epitopes to an effective vaccine. Peptides can be readily synthesized chemically and easily purified utilizing reagents free of contaminating bacteria or animal substances. The small size allows a clear focus on the mutated region of the protein and also reduces irrelevant antigenic competition from other components (non-mutated protein or viral vector antigens).

Yet another example of translating peptide sequencing information into a therapeutic vaccine may include a combination with a strong vaccine adjuvant. Effective vaccines may require a strong adjuvant to initiate an immune response. For example, poly-ICLC, an agonist of TLR3 and the RNA helicase-domains of MDA5 and RIG3, has shown several desirable properties for a vaccine adjuvant. These properties include the induction of local and systemic activation of immune cells in vivo, production of stimulatory chemokines and cytokines, and stimulation of antigen-presentation by DCs. Furthermore, poly-ICLC can induce durable CD4⁺ and CD8⁺ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NYESO-1 peptide vaccine (in addition to Montanide) showed induction of CD4⁺ and CD8⁺ T cell, as well as antibody responses to the peptide in a recent phase 1 study. At the same time, poly-ICLC has been extensively tested in more than 25 clinical trials to date and exhibited a relatively benign toxicity profile.

In some aspects, provided herein is a composition comprising: a first peptide comprising a first neoepitope of a protein and a second peptide comprising a second neoepitope of the same protein, a polynucleotide encoding the first peptide and the second peptide, one or more APCs comprising the first peptide and the second peptide, or a first T cell receptor (TCR) specific for the first neoepitope in complex with an HLA protein and a second TCR specific for the second neoepitope in complex with an HLA protein; wherein the first peptide is different from the second peptide, and wherein the first neoepitope comprises a mutation and the second neoepitope comprises the same mutation.

In some aspects, provided herein is a composition comprising: a first peptide comprising a first neoepitope of a region of a protein and a second peptide comprising a second neoepitope of the region of the same protein, wherein the first neoepitope and the second neoepitope comprise at least one amino acid of the region that is the same, a polynucleotide encoding the first peptide and the second peptide, on or more APCs comprising the first peptide and the second peptide, or a first T cell receptor (TCR) specific for the first neoepitope in complex with an HLA protein and a second TCR specific for the second neoepitope in complex with an HLA protein; wherein the first peptide is different from the second peptide, and wherein the first neoepitope comprises a first mutation and the second neoepitope comprises a second mutation.

In some embodiments, the first mutation and the second mutation are the same. In some embodiments, the first peptide and the second peptide are different molecules. In some embodiments, the first neoepitope comprises a first neoepitope of a region of the same protein, wherein the second neoepitope comprises a second neoepitope of the region of the same protein. In some embodiments, the first neoepitope and the second neoepitope comprise at least one amino acid of the region that is the same. In some embodiments, the region of the protein comprises at least 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, 600, 700, 800, 900, or 1,000 contiguous amino acids of the protein. In some embodiments, the region of the protein comprises at most 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, 600, 700, 800, 900, or 1,000 contiguous amino acids of the protein. In some embodiments, the first neoepitope binds to a class I HLA protein to form a class I HLA-peptide complex. In some embodiments, the second neoepitope binds to a class II HLA protein to form a class II HLA-peptide complex. In some embodiments, the second neoepitope binds to a class I HLA protein to form a class I HLA-peptide complex. In some embodiments, the first neoepitope binds to a class II HLA protein to form a class II HLA-peptide complex. In some embodiments, the first neoepitope is a first neoepitope peptide processed from the first peptide and/or the second neoepitope is a second neoepitope peptide processed from the second peptide. In some embodiments, the first neoepitope is shorter in length than first peptide and/or the second neoepitope is shorter in length than second peptide. In some embodiments, the first neoepitope peptide is processed by an antigen presenting cell (APC) comprising the first peptide and/or the second neoepitope peptide is processed by an APC comprising the second peptide. In some embodiments, the first neoepitope activates CD8⁺ T cells. In some embodiments, the second neoepitope activates CD4⁺ T cells. In some embodiments, the second neoepitope activates CD8⁺ T cells. In some embodiments, the first neoepitope activates CD4⁺ T cells. In some embodiments, a TCR of a CD4⁺ T cell binds to a class II HLA-peptide complex comprising the first or second peptide. In some embodiments, a TCR of a CD8⁺ T cell binds to a class I HLA-peptide complex comprising the first or second peptide. In some embodiments, a TCR of a CD4⁺ T cell binds to a class I HLA-peptide complex comprising the first or second peptide. In some embodiments, a TCR of a CD8⁺ T cell binds to a class II HLA-peptide complex comprising the first or second peptide. In some embodiments, the one or more APCs comprise a first APC comprising the first peptide and a second APC comprising the second peptide. In some embodiments, the mutation is selected from the group consisting of a point mutation, a splice-site mutation, a frameshift mutation, a read-through mutation, a gene fusion mutation and any combination thereof. In some embodiments, the first neoepitope and the second neoepitope comprises a sequence encoded by a gene of Table 1 or 2. In some embodiments, the protein is encoded by a gene of Table 1 or 2. In some embodiments, the mutation is a mutation of column 2 of Table 1 or 2. In some embodiments, the protein is GATA3. In some embodiments, the first neoepitope and the second neoepitope comprises a sequence encoded by a gene of Table 34 or Table 36. In some embodiments, the protein is encoded by a gene of Table 34 or Table 36. In some embodiments, the mutation is a mutation of column 2 of Table 34 or Table 36. In some embodiments, the protein is BTK. In some embodiments, the first neoepitope and the second neoepitope comprises a sequence encoded by a gene of Table 40A-40D. In some embodiments, the protein is encoded by a gene of Table 3 or 35. In some embodiments, the mutation is a mutation of column 2 of Table 3 or 35. In some embodiments, the protein is EGFR. In some embodiments, a single polypeptide comprises the first peptide and the second peptide, or a single polynucleotide encodes the first peptide and the second peptide. In some embodiments, the first peptide and the second peptide are encoded by a sequence transcribed from a same transcription start site. In some embodiments, the first peptide is encoded by a sequence transcribed from a first transcription start site and the second peptide is encoded by a sequence transcribed from a second transcription start site. In some embodiments, the single polypeptide has a length of at least 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the polypeptide comprises a first sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to a first corresponding wild-type sequence; and a second sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to a corresponding second wild-type sequence. In some embodiments, the polypeptide comprises a first sequence of at least 8 or 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to a corresponding first wild-type sequence; and a second sequence of at least 16 or 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to a corresponding second wild-type sequence. In some embodiments, the second peptide is longer than the first peptide In some embodiments, the first peptide is longer than the second peptide. In some embodiments, the first peptide has a length of at least 9; 10; 11; 12; 13; 14; 15; 16; 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the second peptide has a length of at least 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the first peptide comprises a sequence of at least 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a corresponding wild-type sequence. In some embodiments, the second peptide comprises a sequence of at least 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a corresponding wild-type sequence. In some embodiments, the second neoepitope is longer than the first neoepitope. In some embodiments, the first neoepitope has a length of at least 8 amino acids. In some embodiments, the first neoepitope has a length of from 8 to 12 amino acids. In some embodiments, the first neoepitope comprises a sequence of at least 8 contiguous amino acids, wherein at least 2 of the 8 contiguous amino acids are different at corresponding positions of a wild-type sequence. In some embodiments, the second neoepitope has a length of at least 16 amino acids. In some embodiments, the second neoepitope has a length of from 16 to 25 amino acids. In some embodiments, the second neoepitope comprises a sequence of at least 16 contiguous amino acids, wherein at least 2 of the 16 contiguous amino acids are different at corresponding positions of a wild-type sequence.

In some embodiments, the first peptide comprises at least one an additional mutation. In some embodiments, one or more of the at least one additional mutation is not a mutation in the first neoepitope. In some embodiments, one or more of the at least one additional mutation is a mutation in the first neoepitope. In some embodiments, the second peptide comprises at least one additional mutation. In some embodiments, one or more of the at least one additional mutation is not a mutation in the second neoepitope. In some embodiments, one or more of the at least one additional mutation is a mutation in the second neoepitope. In some embodiments, the first peptide, the second peptide or both comprise at least one flanking sequence, wherein the at least one flanking sequence is upstream or downstream of the neoepitope. In some embodiments, the at least one flanking sequence has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a corresponding wild-type sequence. In some embodiments, the at least one flanking sequence comprises a non-wild-type sequence. In some embodiments, the at least one flanking sequence is a N-terminus flanking sequence. In some embodiments, the at least one flanking sequence is a C-terminus flanking sequence. In some embodiments, the at least one flanking sequence of the first peptide has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the at least one flanking sequence of the second peptide. In some embodiments, the at least one flanking region of the first peptide is different from the at least one flanking region of the second peptide. In some embodiments, the at least one flanking residue comprises the mutation. In some embodiments, the first neoepitope, the second neoepitope or both comprises at least one anchor residue. In some embodiments, the at least one anchor residue of the first neoepitope is at a canonical anchor position. In some embodiments, the at least one anchor residue of the first neoepitope is at a non-canonical anchor position. In some embodiments, the at least one anchor residue of the second neoepitope is at a canonical anchor position. In some embodiments, the at least one anchor residue of the second neoepitope is at a non-canonical anchor position. In some embodiments, the at least one anchor residue of the first neoepitope is different from the at least one anchor residue of the second neoepitope. In some embodiments, the at least one anchor residue is a wild-type residue. In some embodiments, the at least one anchor residue is a substitution. In some embodiments, the first neoepitope and/or the second neoepitope binds to an HLA protein with a greater affinity than a corresponding neoepitope without the substitution. In some embodiments, the first neoepitope and/or the second neoepitope binds to an HLA protein with a greater affinity than a corresponding wild-type sequence without the substitution. In some embodiments, at least one anchor residue does not comprise the mutation. In some embodiments, the first neoepitope, the second neoepitope or both comprise at least one anchor residue flanking region. In some embodiments, the neoepitope comprises at least one anchor residue. In some embodiments, the at least one anchor residues comprises at least two anchor residues. In some embodiments, the at least two anchor residues are separated by a separation region comprising at least 1 amino acid. In some embodiments, the at least one anchor residue flanking region is not within the separation region. In some embodiments, the at least one anchor residue flanking region is upstream of a N-terminal anchor residue of the at least two anchor residues downstream of a C-terminal anchor residue of the at least two anchor residue both (a) and (b).

In some embodiments, composition comprises an adjuvant. In some embodiments, the composition comprises one or more additional peptides, wherein the one or more additional peptides comprise a third neoepitope. In some embodiments, the first and/or second neoepitope binds to an HLA protein with a greater affinity than a corresponding wild-type sequence. In some embodiments, the first and/or second neoepitope binds to an HLA protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class I protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class II protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to a protein encoded by an HLA allele expressed by a subject. In some embodiments, the mutation is not present in non-cancer cells of a subject. In some embodiments, the first and/or second neoepitope is encoded by a gene or an expressed gene of a subject's cancer cells. In some embodiments, the composition comprises a first T cell comprising the first TCR. In some embodiments, the composition comprises a second T cell comprising the second TCR. In some embodiments, the first TCR comprises a non-native intracellular domain and/or the second TCR comprises a non-native intracellular domain. In some embodiments, the first TCR is a soluble TCR and/or the second TCR is a soluble TCR. In some embodiments, the first and/or second T cell is a cytotoxic T cell. In some embodiments, the first and/or second T cell is a gamma delta T cell. In some embodiments, the first and/or second T cell is a helper T cell. In some embodiments, the first T cell is a T cell stimulated, expanded or induced with the first neoepitope and/or the second T cell is a T cell stimulated, expanded or induced with the second neoepitope. In some embodiments, the first and/or second T cell is an autologous T cell. In some embodiments, the first and/or second T cell is an allogenic T cell. In some embodiments, the first and/or second T cell is an engineered T cell. In some embodiments, the first and/or second T cell is a T cell of a cell line. In some embodiments, the first and/or second TCR binds to an HLA-peptide complex with a K_{D} or an IC₅₀ of less than 1000 nM, 900 nM, 800 nM, 700 nM, 600 nM, 500 nM, 250 nM, 150 nM, 100 nM, 50 nM, 25 nM or 10 nM. In some aspects, provided herein is a vector comprising a polynucleotide encoding a first and a second peptide described herein. In some embodiments, the polynucleotide is operably linked to a promoter. In some embodiments, the vector is a self-amplifying RNA replicon, plasmid, phage, transposon, cosmid, virus, or virion. In some embodiments, the vector is a viral vector. In some embodiments, the vector is derived from a retrovirus, lentivirus, adenovirus, adeno-associated virus, herpes virus, pox virus, alpha virus, vaccinia virus, hepatitis B virus, human papillomavirus or a pseudotype thereof. In some embodiments, the vector is a non-viral vector. In some embodiments, the non-viral vector is a nanoparticle, a cationic lipid, a cationic polymer, a metallic nanopolymer, a nanorod, a liposome, a micelle, a microbubble, a cell-penetrating peptide, or a liposphere.

In some aspects, provided herein is a pharmaceutical composition comprising: a composition described herein, or a vector described herein; and a pharmaceutically acceptable excipient.

In some embodiments, the plurality of cells is autologous cells. In some embodiments, the plurality of APC cells is autologous cells. In some embodiments, the plurality of T cells is autologous cells. In some embodiments, the pharmaceutical composition further comprises an immunomodulatory agent or an adjuvant. In some embodiments, the immunomodulatory agent is a cytokine. In some embodiments, the adjuvant is polyICLC. In some embodiments, the adjuvant is Hiltonol.

In some aspects, provided herein is a method of treating cancer, the method comprising administering to a subject in need thereof a pharmaceutical composition described herein.

In some aspects, provided herein is a method of preventing resistance to a cancer therapy, the method comprising administering to a subject in need thereof a pharmaceutical composition described herein.

In some aspects, provided herein is a method of inducing an immune response, the method comprising administering to a subject in need thereof a pharmaceutical composition described herein.

In some embodiments, the immune response is a humoral response. In some embodiments, the first peptide and the second peptide are administered simultaneously, separately or sequentially. In some embodiments, the first peptide is sequentially administered after the second peptide. In some embodiments, the second peptide is sequentially administered after the first peptide. In some embodiments, the first peptide is sequentially administered after a time period sufficient for the second peptide to activate the T cells. In some embodiments, the second peptide is sequentially administered after a time period sufficient for the first peptide to activate the T cells. In some embodiments, the first peptide is sequentially administered after the second peptide to restimulate the T cells. In some embodiments, the second peptide is sequentially administered after the first peptide to restimulate the T cells. In some embodiments, the first peptide is administered to stimulate the T cells and the second peptide is administered after the first peptide to restimulate the T cells. In some embodiments, the second peptide is administered to stimulate the T cells and the first peptide is administered after the second peptide to restimulate the T cells.

In some embodiments, the subject has cancer, wherein the cancer is selected from the group consisting of melanoma, ovarian cancer, lung cancer, prostate cancer, breast cancer, colorectal cancer, endometrial cancer, and chronic lymphocytic leukemia (CLL). In some embodiments, the cancer is a breast cancer that is resistant to anti-estrogen therapy, is an MSI breast cancer, is a metastatic breast cancer, is a Her2 negative breast cancer, is a Her2 positive breast cancer, is an ER negative breast cancer, is an ER positive breast cancer, is a PR positive breast cancer, is a PR negetive breast cancer or any combination thereof. In some embodiments, the breast cancer expresses an estrogen receptor with a mutation. In some embodiments, the subject has a breast cancer that is resistant to anti-estrogen therapy. In some embodiments, the breast cancer expresses an estrogen receptor with a mutation. In some embodiments, the subject has a CLL that is resistant to ibrutinib therapy. In some embodiments, the CLL expresses a Bruton tyrosine kinase with a mutation, such as a C481S mutation. In some embodiments, the subject has a lung cancer that is resistant to a tyrosine kinase inhibitor. In some embodiments, the lung cancer expresses an epidermal growth factor receptor (EGFR) with a mutation, such as a T790M mutation. In some embodiments, the plurality of APC cells comprising the first peptide and the plurality of APC cells comprising the second peptide are administered simultaneously, separately or sequentially. In some embodiments, the plurality of T cells comprising the first TCR and the plurality of T cells comprising the second TCR are administered simultaneously, separately or sequentially. In some embodiments, the method further comprises administering at least one additional therapeutic agent or modality. In some embodiments, the at least one additional therapeutic agent or modality is surgery, a checkpoint inhibitor, an antibody or fragment thereof, a chemotherapeutic agent, radiation, a vaccine, a small molecule, a T cell, a vector, and APC, a polynucleotide, an oncolytic virus or any combination thereof. In some embodiments, the at least one additional therapeutic agent is an anti-PD-1 agent and anti-PD-L1 agent, an anti-CTLA-4 agent, or an anti-CD40 agent. In some embodiments, the additional therapeutic agent is administered before, simultaneously, or after administering a pharmaceutical composition according described herein.

### Peptides

In aspects, the present disclosure provides isolated peptides that comprise a tumor specific mutation from Table 1 or 2. In aspects, the present disclosure provides isolated peptides that comprise a tumor specific mutation from Table 34. In aspects, the present disclosure provides isolated peptides that comprise a tumor specific mutation from Table 40A-40D. These peptides and polypeptides are referred to herein as “neoantigenic peptides” or “neoantigenic polypeptides”. “Polypeptide”, “peptide” and their grammatical equivalents as used herein refer to a polymer of amino acid residues, typically L-amino acids, connected one to the other, typically by peptide bonds between the α-amino and carboxyl groups of adjacent amino acids. Polypeptides and peptides include, but are not limited to, “mutant peptides”, “neoantigen peptides” and “neoantigenic peptides”, Polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. A peptide or polypeptide may comprise at least one flanking sequence. The term “flanking sequence” as used herein refers to a fragment or region of a peptide that is not a part of an epitope.

TABLE 1
lists GATA3 neoORF Peptides

| Type | Sequences |
|---|---|
| 8 mers | EPCSMLTG, PCSMLTGP, CSMLTGPP, SMLTGPPA, MLTGPPAR, LTGPPARV, TGPPARVP, |
| | GPPARVPA, PPARVPAV, PARVPAVP, ARVPAVPF, RVPAVPFD, VPAVPFDL, PAVPFDLH, |
| | AVPFDLHF, VPFDLHFC, PFDLHFCR, FDLHFCRS, DLHFCRSS, LHFCRSSI, HFCRSSIM, |
| | FCRSSIMK, CRSSIMKP, RSSIMKPK, SSIMKPKR, SIMKPKRD, IMKPKRDG, MKPKRDGY, |
| | KPKRDGYM, PKRDGYMF, KRDGYMFL, RDGYMFLK, DGYMFLKA, GYMFLKAE, |
| | YMFLKAES, MFLKAESK, FLKAESKI, LKAESKIM, KAESKIMF, AESKIMFA, ESKIMFAT, |
| | SKIMFATL, KIMFATLQ, IMFATLQR, MFATLQRS, FATLQRSS, ATLQRSSL, TLQRSSLW, |
| | LQRSSLWC, QRSSLWCL, RSSLWCLC, SSLWCLCS, SLWCLCSN |
| 9 mers | EPCSMLTGP, PCSMLTGPP, CSMLTGPPA, SMLTGPPAR, MLTGPPARV, LTGPPARVP, |
| | TGPPARVPA, GPPARVPAV, PPARVPAVP, PARVPAVPF, ARVPAVPFD, RVPAVPFDL, |
| | VPAVPFDLH, PAVPFDLHF, AVPFDLHFC, VPFDLHFCR, PFDLHFCRS, FDLHFCRSS, |
| | DLHFCRSSI, LHFCRSSIM, HFCRSSIMK, FCRSSIMKP, CRSSIMKPK, RSSIMKPKR, |
| | SSIMKPKRD, SIMKPKRDG, IMKPKRDGY, MKPKRDGYM, KPKRDGYMF, PKRDGYMFL, |
| | KRDGYMFLK, RDGYMFLKA, DGYMFLKAE, GYMFLKAES, YMFLKAESK, MFLKAESKI, |
| | FLKAESKIM, LKAESKIMF, KAESKIMFA, AESKIMFAT, ESKIMFATL, SKIMFATLQ, |
| | KIMFATLQR, IMFATLQRS, MFATLQRSS, FATLQRSSL, ATLQRSSLW, TLQRSSLWC, |
| | LQRSSLWCL, QRSSLWCLC, RSSLWCLCS, SSLWCLCSN, SLWCLCSNH |
| 10 mers | EPCSMLTGPP, PCSMLTGPPA, CSMLTGPPAR, SMLTGPPARV, MLTGPPARVP, |
| | LTGPPARVPA, TGPPARVPAV, GPPARVPAVP, PPARVPAVPF, PARVPAVPFD, |
| | ARVPAVPFDL, RVPAVPFDLH, VPAVPFDLHF, PAVPFDLHFC, AVPFDLHFCR, |
| | VPFDLHFCRS, PFDLHFCRSS, FDLHFCRSSI, DLHFCRSSIM, LHFCRSSIMK, |
| | HFCRSSIMKP, FCRSSIMKPK, CRSSIMKPKR, RSSIMKPKRD, SSIMKPKRDG, |
| | SIMKPKRDGY, IMKPKRDGYM, MKPKRDGYMF, KPKRDGYMFL, PKRDGYMFLK, |
| | KRDGYMFLKA, RDGYMFLKAE, DGYMFLKAES, GYMFLKAESK, YMFLKAESKI, |
| | MFLKAESKIM, FLKAESKIMF, LKAESKIMFA, KAESKIMFAT, AESKIMFATL, |
| | ESKIMFATLQ, SKIMFATLQR, KIMFATLQRS, IMFATLQRSS, MFATLQRSSL, |
| | FATLQRSSLW, ATLQRSSLWC, TLQRSSLWCL, LQRSSLWCLC, QRSSLWCLCS, |
| | RSSLWCLCSN, SSLWCLCSNH, SLWCLCSNH |
| 11 mers | EPCSMLTGPPA, PCSMLTGPPAR, CSMLTGPPARV, SMLTGPPARVP, MLTGPPARVPA, |
| | LTGPPARVPAV, TGPPARVPAVP, GPPARVPAVPF, PPARVPAVPFD, PARVPAVPFDL, |
| | ARVPAVPFDLH, RVPAVPFDLHF, VPAVPFDLHFC, PAVPFDLHFCR, AVPFDLHFCRS, |
| | VPFDLHFCRSS, PFDLHFCRSSI, FDLHFCRSSIM, DLHFCRSSIMK, LHFCRSSIMKP, |
| | HFCRSSIMKPK, FCRSSIMKPKR, CRSSIMKPKRD, RSSIMKPKRDG, SSIMKPKRDGY, |
| | SIMKPKRDGYM, IMKPKRDGYMF, MKPKRDGYMFL, KPKRDGYMFLK, |
| | PKRDGYMFLKA, KRDGYMFLKAE, RDGYMFLKAES, DGYMFLKAESK, GYMFLKAESKI, |
| | YMFLKAESKIM, MFLKAESKIMF, FLKAESKIMFA, LKAESKIMFAT, KAESKIMFATL, |
| | AESKIMFATLQ, ESKIMFATLQR, SKIMFATLQRS, KIMFATLQRSS, IMFATLQRSSL, |
| | MFATLQRSSLW, FATLQRSSLWC, ATLQRSSLWCL, TLQRSSLWCLC, LQRSSLWCLCS, |
| | QRSSLWCLCSN, RSSLWCLCSNH |
| 12 mers | EPCSMLTGPPAR, PCSMLTGPPARV, CSMLTGPPARVP, SMLTGPPARVPA, |
| | MLTGPPARVPAV, LTGPPARVPAVP, TGPPARVPAVPF, GPPARVPAVPFD, |
| | PPARVPAVPFDL, PARVPAVPFDLH, ARVPAVPFDLHF, RVPAVPFDLHFC, |
| | VPAVPFDLHFCR, PAVPFDLHFCRS, AVPFDLHFCRSS, VPFDLHFCRSSI, PFDLHFCRSSIM, |
| | FDLHFCRSSIMK, DLHFCRSSIMKP, LHFCRSSIMKPK, HFCRSSIMKPKR, FCRSSIMKPKRD, |
| | CRSSIMKPKRDG, RSSIMKPKRDGY, SSIMKPKRDGYM, SIMKPKRDGYMF, |
| | IMKPKRDGYMFL, MKPKRDGYMFLK, KPKRDGYMFLKA, PKRDGYMFLKAE, |
| | KRDGYMFLKAES, RDGYMFLKAESK, DGYMFLKAESKI, GYMFLKAESKIM, |
| | YMFLKAESKIMF, MFLKAESKIMFA, FLKAESKIMFAT, LKAESKIMFATL, |
| | KAESKIMFATLQ, AESKIMFATLQR, ESKIMFATLQRS, SKIMFATLQRSS, KIMFATLQRSSL, |
| | IMFATLQRSSLW, MFATLQRSSLWC, FATLQRSSLWCL, ATLQRSSLWCLC, |
| | TLQRSSLWCLCS, LQRSSLWCLCSN, QRSSLWCLCSNH |
| 13 mers | EPCSMLTGPPARV, PCSMLTGPPARVP, CSMLTGPPARVPA, SMLTGPPARVPAV, |
| | MLTGPPARVPAVP, LTGPPARVPAVPF, TGPPARVPAVPFD, GPPARVPAVPFDL, |
| | PPARVPAVPFDLH, PARVPAVPFDLHF, ARVPAVPFDLHFC, RVPAVPFDLHFCR, |
| | VPAVPFDLHFCRS, PAVPFDLHFCRSS, AVPFDLHFCRSSI, VPFDLHFCRSSIM, |
| | PFDLHFCRSSIMK, FDLHFCRSSIMKP, DLHFCRSSIMKPK, LHFCRSSIMKPKR, |
| | HFCRSSIMKPKRD, FCRSSIMKPKRDG, CRSSIMKPKRDGY, RSSIMKPKRDGYM, |
| | SSIMKPKRDGYMF, SIMKPKRDGYMFL, IMKPKRDGYMFLK, MKPKRDGYMFLKA, |
| | KPKRDGYMFLKAE, PKRDGYMFLKAES, KRDGYMFLKAESK, RDGYMFLKAESKI, |
| | DGYMFLKAESKIM, GYMFLKAESKIMF, YMFLKAESKIMFA, MFLKAESKIMFAT, |
| | FLKAESKIMFATL, LKAESKIMFATLQ, KAESKIMFATLQR, AESKIMFATLQRS, |
| | ESKIMFATLQRSS, SKIMFATLQRSSL, KIMFATLQRSSLW, IMFATLQRSSLWC, |
| | MFATLQRSSLWCL, FATLQRSSLWCLC, ATLQRSSLWCLCS, TLQRSSLWCLCSN, |
| | LQRSSLWCLCSNH |
| 14 mers | EPCSMLTGPPARVP, PCSMLTGPPARVPA, CSMLTGPPARVPAV, SMLTGPPARVPAVP, |
| | MLTGPPARVPAVPF, LTGPPARVPAVPFD, TGPPARVPAVPFDL, GPPARVPAVPFDLH, |
| | PPARVPAVPFDLHF, PARVPAVPFDLHFC, ARVPAVPFDLHFCR, RVPAVPFDLHFCRS, |
| | VPAVPFDLHFCRSS, PAVPFDLHFCRSSI, AVPFDLHFCRSSIM, VPFDLHFCRSSIMK, |
| | PFDLHFCRSSIMKP, FDLHFCRSSIMKPK, DLHFCRSSIMKPKR, LHFCRSSIMKPKRD, |
| | HFCRSSIMKPKRDG, FCRSSIMKPKRDGY, CRSSIMKPKRDGYM, RSSIMKPKRDGYMF, |
| | SSIMKPKRDGYMFL, SIMKPKRDGYMFLK, IMKPKRDGYMFLKA, MKPKRDGYMFLKAE, |
| | KPKRDGYMFLKAES, PKRDGYMFLKAESK, KRDGYMFLKAESKI, RDGYMFLKAESKIM, |
| | DGYMFLKAESKIMF, GYMFLKAESKIMFA, YMFLKAESKIMFAT, MFLKAESKIMFATL, |
| | FLKAESKIMFATLQ, LKAESKIMFATLQR, KAESKIMFATLQRS, AESKIMFATLQRSS, |
| | ESKIMFATLQRSSL, SKIMFATLQRSSLW, KIMFATLQRSSLWC, IMFATLQRSSLWCL, |
| | MFATLQRSSLWCLC, FATLQRSSLWCLCS, ATLQRSSLWCLCSN, TLQRSSLWCLCSNH |
| 15 mers | EPCSMLTGPPARVPA, PCSMLTGPPARVPAV, CSMLTGPPARVPAVP, |
| | SMLTGPPARVPAVPF, MLTGPPARVPAVPFD, LTGPPARVPAVPFDL, |
| | TGPPARVPAVPFDLH, GPPARVPAVPFDLHF, PPARVPAVPFDLHFC, PARVPAVPFDLHFCR, |
| | ARVPAVPFDLHFCRS, RVPAVPFDLHFCRSS, VPAVPFDLHFCRSSI, PAVPFDLHFCRSSIM, |
| | AVPFDLHFCRSSIMK. VPFDLHFCRSSIMKP, PFDLHFCRSSIMKPK, FDLHFCRSSIMKPKR, |
| | DLHFCRSSIMKPKRD, LHFCRSSIMKPKRDG, HFCRSSIMKPKRDGY, |
| | FCRSSIMKPKRDGYM, CRSSIMKPKRDGYMF, RSSIMKPKRDGYMFL, |
| | SSIMKPKRDGYMFLK, SIMKPKRDGYMFLKA, IMKPKRDGYMFLKAE, |
| | MKPKRDGYMFLKAES, KPKRDGYMFLKAESK, PKRDGYMFLKAESKI, |
| | KRDGYMFLKAESKIM, RDGYMFLKAESKIMF, DGYMFLKAESKIMFA, |
| | GYMFLKAESKIMFAT, YMFLKAESKIMFATL, MFLKAESKIMFATLQ, |
| | FLKAESKIMFATLQR, LKAESKIMFATLQRS, KAESKIMFATLQRSS, AESKIMFATLQRSSL, |
| | ESKIMFATLQRSSLW, SKIMFATLQRSSLWC, KIMFATLQRSSLWCL, |
| | IMFATLQRSSLWCLC, MFATLQRSSLWCLCS, FATLQRSSLWCLCSN, |
| | ATLQRSSLWCLCSNH |
| 16 mers | EPCSMLTGPPARVPAV, PCSMLTGPPARVPAVP, CSMLTGPPARVPAVPF, |
| | SMLTGPPARVPAVPFD, MLTGPPARVPAVPFDL, LTGPPARVPAVPFDLH, |
| | TGPPARVPAVPFDLHF, GPPARVPAVPFDLHFC, PPARVPAVPFDLHFCR, |
| | PARVPAVPFDLHFCRS, ARVPAVPFDLHFCRSS, RVPAVPFDLHFCRSSI, |
| | VPAVPFDLHFCRSSIM, PAVPFDLHFCRSSIMK, AVPFDLHFCRSSIMKP, |
| | VPFDLHFCRSSIMKPK, PFDLHFCRSSIMKPKR, FDLHFCRSSIMKPKRD, |
| | DLHFCRSSIMKPKRDG, LHFCRSSIMKPKRDGY, HFCRSSIMKPKRDGYM, |
| | FCRSSIMKPKRDGYMF, CRSSIMKPKRDGYMFL, RSSIMKPKRDGYMFLK, |
| | SSIMKPKRDGYMFLKA, SIMKPKRDGYMFLKAE, IMKPKRDGYMFLKAES, |
| | MKPKRDGYMFLKAESK, KPKRDGYMFLKAESKI, PKRDGYMFLKAESKIM, |
| | KRDGYMFLKAESKIMF, RDGYMFLKAESKIMFA, DGYMFLKAESKIMFAT, |
| | GYMFLKAESKIMFATL, YMFLKAESKIMFATLQ, MFLKAESKIMFATLQR, |
| | FLKAESKIMFATLQRS, LKAESKIMFATLQRSS, KAESKIMFATLQRSSL, |
| | AESKIMFATLQRSSLW, ESKIMFATLQRSSLWC, SKIMFATLQRSSLWCL, |
| | KIMFATLQRSSLWCLC, IMFATLQRSSLWCLCS, MFATLQRSSLWCLCSN, |
| | FATLQRSSLWCLCSNH |
| 17 mers | EPCSMLTGPPARVPAVP, PCSMLTGPPARVPAVPF, CSMLTGPPARVPAVPFD, |
| | SMLTGPPARVPAVPFDL, MLTGPPARVPAVPFDLH, LTGPPARVPAVPFDLHF, |
| | TGPPARVPAVPFDLHFC, GPPARVPAVPFDLHFCR, PPARVPAVPFDLHFCRS, |
| | PARVPAVPFDLHFCRSS, ARVPAVPFDLHFCRSSI, RVPAVPFDLHFCRSSIM, |
| | VPAVPFDLHFCRSSIMK, PAVPFDLHFCRSSIMKP, AVPFDLHFCRSSIMKPK, |
| | VPFDLHFCRSSIMKPKR, PFDLHFCRSSIMKPKRD, FDLHFCRSSIMKPKRDG, |
| | DLHFCRSSIMKPKRDGY, LHFCRSSIMKPKRDGYM, HFCRSSIMKPKRDGYMF, |
| | FCRSSIMKPKRDGYMFL, CRSSIMKPKRDGYMFLK, RSSIMKPKRDGYMFLKA, |
| | SSIMKPKRDGYMFLKAE, SIMKPKRDGYMFLKAES, IMKPKRDGYMFLKAESK, |
| | MKPKRDGYMFLKAESKI, KPKRDGYMFLKAESKIM, PKRDGYMFLKAESKIMF, |
| | KRDGYMFLKAESKIMFA, RDGYMFLKAESKIMFAT, DGYMFLKAESKIMFATL, |
| | GYMFLKAESKIMFATLQ, YMFLKAESKIMFATLQR, MFLKAESKIMFATLQRS, |
| | FLKAESKIMFATLQRSS, LKAESKIMFATLQRSSL, KAESKIMFATLQRSSLW, |
| | AESKIMFATLQRSSLWC, ESKIMFATLQRSSLWCL, SKIMFATLQRSSLWCLC, |
| | KIMFATLQRSSLWCLCS, IMFATLQRSSLWCLCSN, MFATLQRSSLWCLCSNH |
| 18 mers | EPCSMLTGPPARVPAVPF, PCSMLTGPPARVPAVPFD, CSMLTGPPARVPAVPFDL, |
| | SMLTGPPARVPAVPFDLH, MLTGPPARVPAVPFDLHF, LTGPPARVPAVPFDLHFC, |
| | TGPPARVPAVPFDLHFCR, GPPARVPAVPFDLHFCRS, PPARVPAVPFDLHFCRSS, |
| | PARVPAVPFDLHFCRSSI, ARVPAVPFDLHFCRSSIM, RVPAVPFDLHFCRSSIMK, |
| | VPAVPFDLHFCRSSIMKP, PAVPFDLHFCRSSIMKPK, AVPFDLHFCRSSIMKPKR, |
| | VPFDLHFCRSSIMKPKRD, PFDLHFCRSSIMKPKRDG, FDLHFCRSSIMKPKRDGY, |
| | DLHFCRSSIMKPKRDGYM, LHFCRSSIMKPKRDGYMF, HFCRSSIMKPKRDGYMFL, |
| | FCRSSIMKPKRDGYMFLK, CRSSIMKPKRDGYMFLKA, RSSIMKPKRDGYMFLKAE, |
| | SSIMKPKRDGYMFLKAES, SIMKPKRDGYMFLKAESK, IMKPKRDGYMFLKAESKI, |
| | MKPKRDGYMFLKAESKIM, KPKRDGYMFLKAESKIMF, PKRDGYMFLKAESKIMFA, |
| | KRDGYMFLKAESKIMFAT, RDGYMFLKAESKIMFATL, DGYMFLKAESKIMFATLQ, |
| | GYMFLKAESKIMFATLQR, YMFLKAESKIMFATLQRS, MFLKAESKIMFATLQRSS, |
| | FLKAESKIMFATLQRSSL, LKAESKIMFATLQRSSLW, KAESKIMFATLQRSSLWC, |
| | AESKIMFATLQRSSLWCL, ESKIMFATLQRSSLWCLC, SKIMFATLQRSSLWCLCS, |
| | KIMFATLQRSSLWCLCSN, IMFATLQRSSLWCLCSNH |
| 19 mers | EPCSMLTGPPARVPAVPFD, PCSMLTGPPARVPAVPFDL, CSMLTGPPARVPAVPFDLH, |
| | SMLTGPPARVPAVPFDLHF, MLTGPPARVPAVPFDLHFC, LTGPPARVPAVPFDLHFCR, |
| | TGPPARVPAVPFDLHFCRS, GPPARVPAVPFDLHFCRSS, PPARVPAVPFDLHFCRSSI, |
| | PARVPAVPFDLHFCRSSIM, ARVPAVPFDLHFCRSSIMK, RVPAVPFDLHFCRSSIMKP, |
| | VPAVPFDLHFCRSSIMKPK, PAVPFDLHFCRSSIMKPKR, AVPFDLHFCRSSIMKPKRD, |
| | VPFDLHFCRSSIMKPKRDG, PFDLHFCRSSIMKPKRDGY, FDLHFCRSSIMKPKRDGYM, |
| | DLHFCRSSIMKPKRDGYMF, LHFCRSSIMKPKRDGYMFL, HFCRSSIMKPKRDGYMFLK, |
| | FCRSSIMKPKRDGYMFLKA, CRSSIMKPKRDGYMFLKAE, RSSIMKPKRDGYMFLKAES, |
| | SSIMKPKRDGYMFLKAESK, SIMKPKRDGYMFLKAESKI, IMKPKRDGYMFLKAESKIM, |
| | MKPKRDGYMFLKAESKIMF, KPKRDGYMFLKAESKIMFA, PKRDGYMFLKAESKIMFAT, |
| | KRDGYMFLKAESKIMFATL, RDGYMFLKAESKIMFATLQ, DGYMFLKAESKIMFATLQR, |
| | GYMFLKAESKIMFATLQRS, YMFLKAESKIMFATLQRSS, MFLKAESKIMFATLQRSSL, |
| | FLKAESKIMFATLQRSSLW, LKAESKIMFATLQRSSLWC, KAESKIMFATLQRSSLWCL, |
| | AESKIMFATLQRSSLWCLC, ESKIMFATLQRSSLWCLCS, SKIMFATLQRSSLWCLCSN, |
| | KIMFATLQRSSLWCLCSNH |
| 20 mers | EPCSMLTGPPARVPAVPFDL, PCSMLTGPPARVPAVPFDLH, CSMLTGPPARVPAVPFDLHF, |
| | SMLTGPPARVPAVPFDLHFC, MLTGPPARVPAVPFDLHFCR, LTGPPARVPAVPFDLHFCRS, |
| | TGPPARVPAVPFDLHFCRSS, GPPARVPAVPFDLHFCRSSI, PPARVPAVPFDLHFCRSSIM, |
| | PARVPAVPFDLHFCRSSIMK, ARVPAVPFDLHFCRSSIMKP, RVPAVPFDLHFCRSSIMKPK, |
| | VPAVPFDLHFCRSSIMKPKR, PAVPFDLHFCRSSIMKPKRD, AVPFDLHFCRSSIMKPKRDG, |
| | VPFDLHFCRSSIMKPKRDGY, PFDLHFCRSSIMKPKRDGYM, FDLHFCRSSIMKPKRDGYMF, |
| | DLHFCRSSIMKPKRDGYMFL, LHFCRSSIMKPKRDGYMFLK, |
| | HFCRSSIMKPKRDGYMFLKA, FCRSSIMKPKRDGYMFLKAE, |
| | CRSSIMKPKRDGYMFLKAES, RSSIMKPKRDGYMFLKAESK, SSIMKPKRDGYMFLKAESKI, |
| | SIMKPKRDGYMFLKAESKIM, IMKPKRDGYMFLKAESKIMF, |
| | MKPKRDGYMFLKAESKIMFA, KPKRDGYMFLKAESKIMFAT, |
| | PKRDGYMFLKAESKIMFATL, KRDGYMFLKAESKIMFATLQ, |
| | RDGYMFLKAESKIMFATLQR, DGYMFLKAESKIMFATLQRS, |
| | GYMFLKAESKIMFATLQRSS, YMFLKAESKIMFATLQRSSL, MFLKAESKIMFATLQRSSLW, |
| | FLKAESKIMFATLQRSSLWC, LKAESKIMFATLQRSSLWCL, KAESKIMFATLQRSSLWCLC, |
| | AESKIMFATLQRSSLWCLCS, ESKIMFATLQRSSLWCLCSN, SKIMFATLQRSSLWCLCSNH |
| 21 mers | EPCSMLTGPPARVPAVPFDLH, PCSMLTGPPARVPAVPFDLHF, |
| | CSMLTGPPARVPAVPFDLHFC, SMLTGPPARVPAVPFDLHFCR, |
| | MLTGPPARVPAVPFDLHFCRS, LTGPPARVPAVPFDLHFCRSS, |
| | TGPPARVPAVPFDLHFCRSSI, GPPARVPAVPFDLHFCRSSIM, |
| | PPARVPAVPFDLHFCRSSIMK, PARVPAVPFDLHFCRSSIMKP, |
| | ARVPAVPFDLHFCRSSIMKPK, RVPAVPFDLHFCRSSIMKPKR, |
| | VPAVPFDLHFCRSSIMKPKRD, PAVPFDLHFCRSSIMKPKRDG, |
| | AVPFDLHFCRSSIMKPKRDGY, VPFDLHFCRSSIMKPKRDGYM, |
| | PFDLHFCRSSIMKPKRDGYMF, FDLHFCRSSIMKPKRDGYMFL, |
| | DLHFCRSSIMKPKRDGYMFLK, LHFCRSSIMKPKRDGYMFLKA, |
| | HFCRSSIMKPKRDGYMFLKAE, FCRSSIMKPKRDGYMFLKAES, |
| | CRSSIMKPKRDGYMFLKAESK, RSSIMKPKRDGYMFLKAESKI, |
| | SSIMKPKRDGYMFLKAESKIM, SIMKPKRDGYMFLKAESKIMF, |
| | IMKPKRDGYMFLKAESKIMFA, MKPKRDGYMFLKAESKIMFAT, |
| | KPKRDGYMFLKAESKIMFATL, PKRDGYMFLKAESKIMFATLQ, |
| | KRDGYMFLKAESKIMFATLQR, RDGYMFLKAESKIMFATLQRS, |
| | DGYMFLKAESKIMFATLQRSS, GYMFLKAESKIMFATLQRSSL, |
| | YMFLKAESKIMFATLQRSSLW, MFLKAESKIMFATLQRSSLWC, |
| | FLKAESKIMFATLQRSSLWCL, LKAESKIMFATLQRSSLWCLC, |
| | KAESKIMFATLQRSSLWCLCS, AESKIMFATLQRSSLWCLCSN, |
| | ESKIMFATLQRSSLWCLCSNH |
| 22 mers | EPCSMLTGPPARVPAVPFDLHF, PCSMLTGPPARVPAVPFDLHFC, |
| | CSMLTGPPARVPAVPFDLHFCR, SMLTGPPARVPAVPFDLHFCRS, |
| | MLTGPPARVPAVPFDLHFCRSS, LTGPPARVPAVPFDLHFCRSSI, |
| | TGPPARVPAVPFDLHFCRSSIM, GPPARVPAVPFDLHFCRSSIMK, |
| | PPARVPAVPFDLHFCRSSIMKP, PARVPAVPFDLHFCRSSIMKPK, |
| | ARVPAVPFDLHFCRSSIMKPKR, RVPAVPFDLHFCRSSIMKPKRD, |
| | VPAVPFDLHFCRSSIMKPKRDG, PAVPFDLHFCRSSIMKPKRDGY, |
| | AVPFDLHFCRSSIMKPKRDGYM, VPFDLHFCRSSIMKPKRDGYMF, |
| | PFDLHFCRSSIMKPKRDGYMFL, FDLHFCRSSIMKPKRDGYMFLK, |
| | DLHFCRSSIMKPKRDGYMFLKA, LHFCRSSIMKPKRDGYMFLKAE, |
| | HFCRSSIMKPKRDGYMFLKAES, FCRSSIMKPKRDGYMFLKAESK, |
| | CRSSIMKPKRDGYMFLKAESKI, RSSIMKPKRDGYMFLKAESKIM, |
| | SSIMKPKRDGYMFLKAESKIMF, SIMKPKRDGYMFLKAESKIMFA, |
| | IMKPKRDGYMFLKAESKIMFAT, MKPKRDGYMFLKAESKIMFATL, |
| | KPKRDGYMFLKAESKIMFATLQ, PKRDGYMFLKAESKIMFATLQR, |
| | KRDGYMFLKAESKIMFATLQRS, RDGYMFLKAESKIMFATLQRSS, |
| | DGYMFLKAESKIMFATLQRSSL, GYMFLKAESKIMFATLQRSSLW, |
| | YMFLKAESKIMFATLQRSSLWC, MFLKAESKIMFATLQRSSLWCL, |
| | FLKAESKIMFATLQRSSLWCLC, LKAESKIMFATLQRSSLWCLCS, |
| | KAESKIMFATLQRSSLWCLCSN, AESKIMFATLQRSSLWCLCSNH |
| 23 mers | EPCSMLTGPPARVPAVPFDLHFC, PCSMLTGPPARVPAVPFDLHFCR, |
| | CSMLTGPPARVPAVPFDLHFCRS, SMLTGPPARVPAVPFDLHFCRSS, |
| | MLTGPPARVPAVPFDLHFCRSSI, LTGPPARVPAVPFDLHFCRSSIM, |
| | TGPPARVPAVPFDLHFCRSSIMK, GPPARVPAVPFDLHFCRSSIMKP, |
| | PPARVPAVPFDLHFCRSSIMKPK, PARVPAVPFDLHFCRSSIMKPKR, |
| | ARVPAVPFDLHFCRSSIMKPKRD, RVPAVPFDLHFCRSSIMKPKRDG, |
| | VPAVPFDLHFCRSSIMKPKRDGY, PAVPFDLHFCRSSIMKPKRDGYM, |
| | AVPFDLHFCRSSIMKPKRDGYMF, VPFDLHFCRSSIMKPKRDGYMFL, |
| | PFDLHFCRSSIMKPKRDGYMFLK, FDLHFCRSSIMKPKRDGYMFLKA, |
| | DLHFCRSSIMKPKRDGYMFLKAE, LHFCRSSIMKPKRDGYMFLKAES, |
| | HFCRSSIMKPKRDGYMFLKAESK, FCRSSIMKPKRDGYMFLKAESKI, |
| | CRSSIMKPKRDGYMFLKAESKIM, RSSIMKPKRDGYMFLKAESKIMF, |
| | SSIMKPKRDGYMFLKAESKIMFA, SIMKPKRDGYMFLKAESKIMFAT, |
| | IMKPKRDGYMFLKAESKIMFATL, MKPKRDGYMFLKAESKIMFATLQ, |
| | KPKRDGYMFLKAESKIMFATLQR, PKRDGYMFLKAESKIMFATLQRS, |
| | KRDGYMFLKAESKIMFATLQRSS, RDGYMFLKAESKIMFATLQRSSL, |
| | DGYMFLKAESKIMFATLQRSSLW, GYMFLKAESKIMFATLQRSSLWC, |
| | YMFLKAESKIMFATLQRSSLWCL, MFLKAESKIMFATLQRSSLWCLC, |
| | FLKAESKIMFATLQRSSLWCLCS, LKAESKIMFATLQRSSLWCLCSN, |
| | KAESKIMFATLQRSSLWCLCSNH |
| 24 mers | EPCSMLTGPPARVPAVPFDLHFCR, PCSMLTGPPARVPAVPFDLHFCRS, |
| | CSMLTGPPARVPAVPFDLHFCRSS, SMLTGPPARVPAVPFDLHFCRSSI, |
| | MLTGPPARVPAVPFDLHFCRSSIM, LTGPPARVPAVPFDLHFCRSSIMK, |
| | TGPPARVPAVPFDLHFCRSSIMKP, GPPARVPAVPFDLHFCRSSIMKPK, |
| | PPARVPAVPFDLHFCRSSIMKPKR, PARVPAVPFDLHFCRSSIMKPKRD, |
| | ARVPAVPFDLHFCRSSIMKPKRDG, RVPAVPFDLHFCRSSIMKPKRDGY, |
| | VPAVPFDLHFCRSSIMKPKRDGYM, PAVPFDLHFCRSSIMKPKRDGYMF, |
| | AVPFDLHFCRSSIMKPKRDGYMFL, VPFDLHFCRSSIMKPKRDGYMFLK, |
| | PFDLHFCRSSIMKPKRDGYMFLKA, FDLHFCRSSIMKPKRDGYMFLKAE, |
| | DLHFCRSSIMKPKRDGYMFLKAES, LHFCRSSIMKPKRDGYMFLKAESK, |
| | HFCRSSIMKPKRDGYMFLKAESKI, FCRSSIMKPKRDGYMFLKAESKIM, |
| | CRSSIMKPKRDGYMFLKAESKIMF, RSSIMKPKRDGYMFLKAESKIMFA, |
| | SSIMKPKRDGYMFLKAESKIMFAT, SIMKPKRDGYMFLKAESKIMFATL, |
| | IMKPKRDGYMFLKAESKIMFATLQ, MKPKRDGYMFLKAESKIMFATLQR, |
| | KPKRDGYMFLKAESKIMFATLQRS, PKRDGYMFLKAESKIMFATLQRSS, |
| | KRDGYMFLKAESKIMFATLQRSSL, RDGYMFLKAESKIMFATLQRSSLW, |
| | DGYMFLKAESKIMFATLQRSSLWC, GYMFLKAESKIMFATLQRSSLWCL, |
| | YMFLKAESKIMFATLQRSSLWCLC, MFLKAESKIMFATLQRSSLWCLCS, |
| | FLKAESKIMFATLQRSSLWCLCSN, LKAESKIMFATLQRSSLWCLCSNH |
| 25 mers | EPCSMLTGPPARVPAVPFDLHFCRS, PCSMLTGPPARVPAVPFDLHFCRSS, |
| | CSMLTGPPARVPAVPFDLHFCRSSI, SMLTGPPARVPAVPFDLHFCRSSIM, |
| | MLTGPPARVPAVPFDLHFCRSSIMK, LTGPPARVPAVPFDLHFCRSSIMKP, |
| | TGPPARVPAVPFDLHFCRSSIMKPK, GPPARVPAVPFDLHFCRSSIMKPKR, |
| | PPARVPAVPFDLHFCRSSIMKPKRD, PARVPAVPFDLHFCRSSIMKPKRDG, |
| | ARVPAVPFDLHFCRSSIMKPKRDGY, RVPAVPFDLHFCRSSIMKPKRDGYM, |
| | VPAVPFDLHFCRSSIMKPKRDGYMF, PAVPFDLHFCRSSIMKPKRDGYMFL, |
| | AVPFDLHFCRSSIMKPKRDGYMFLK, VPFDLHFCRSSIMKPKRDGYMFLKA, |
| | PFDLHFCRSSIMKPKRDGYMFLKAE, FDLHFCRSSIMKPKRDGYMFLKAES, |
| | DLHFCRSSIMKPKRDGYMFLKAESK, LHFCRSSIMKPKRDGYMFLKAESKI, |
| | HFCRSSIMKPKRDGYMFLKAESKIM, FCRSSIMKPKRDGYMFLKAESKIMF, |
| | CRSSIMKPKRDGYMFLKAESKIMFA, RSSIMKPKRDGYMFLKAESKIMFAT, |
| | SSIMKPKRDGYMFLKAESKIMFATL, SIMKPKRDGYMFLKAESKIMFATLQ, |
| | IMKPKRDGYMFLKAESKIMFATLQR, MKPKRDGYMFLKAESKIMFATLQRS, |
| | KPKRDGYMFLKAESKIMFATLQRSS, PKRDGYMFLKAESKIMFATLQRSSL, |
| | KRDGYMFLKAESKIMFATLQRSSLW, RDGYMFLKAESKIMFATLQRSSLWC, |
| | DGYMFLKAESKIMFATLQRSSLWCL, GYMFLKAESKIMFATLQRSSLWCLC, |
| | YMFLKAESKIMFATLQRSSLWCLCS, MFLKAESKIMFATLQRSSLWCLCSN, |
| | FLKAESKIMFATLQRSSLWCLCSNH |
| 26 mers | EPCSMLTGPPARVPAVPFDLHFCRSS, PCSMLTGPPARVPAVPFDLHFCRSSI, |
| | CSMLTGPPARVPAVPFDLHFCRSSIM, SMLTGPPARVPAVPFDLHFCRSSIMK, |
| | MLTGPPARVPAVPFDLHFCRSSIMKP, LTGPPARVPAVPFDLHFCRSSIMKPK, |
| | TGPPARVPAVPFDLHFCRSSIMKPKR, GPPARVPAVPFDLHFCRSSIMKPKRD, |
| | PPARVPAVPFDLHFCRSSIMKPKRDG, PARVPAVPFDLHFCRSSIMKPKRDGY, |
| | ARVPAVPFDLHFCRSSIMKPKRDGYM, RVPAVPFDLHFCRSSIMKPKRDGYMF, |
| | VPAVPFDLHFCRSSIMKPKRDGYMFL, PAVPFDLHFCRSSIMKPKRDGYMFLK, |
| | AVPFDLHFCRSSIMKPKRDGYMFLKA, VPFDLHFCRSSIMKPKRDGYMFLKAE, |
| | PFDLHFCRSSIMKPKRDGYMFLKAES, FDLHFCRSSIMKPKRDGYMFLKAESK, |
| | DLHFCRSSIMKPKRDGYMFLKAESKI, LHFCRSSIMKPKRDGYMFLKAESKIM, |
| | HFCRSSIMKPKRDGYMFLKAESKIMF, FCRSSIMKPKRDGYMFLKAESKIMFA, |
| | CRSSIMKPKRDGYMFLKAESKIMFAT, RSSIMKPKRDGYMFLKAESKIMFATL, |
| | SSIMKPKRDGYMFLKAESKIMFATLQ, SIMKPKRDGYMFLKAESKIMFATLQR, |
| | IMKPKRDGYMFLKAESKIMFATLQRS, MKPKRDGYMFLKAESKIMFATLQRSS, |
| | KPKRDGYMFLKAESKIMFATLQRSSL, PKRDGYMFLKAESKIMFATLQRSSLW, |
| | KRDGYMFLKAESKIMFATLQRSSLWC, RDGYMFLKAESKIMFATLQRSSLWCL, |
| | DGYMFLKAESKIMFATLQRSSLWCLC, GYMFLKAESKIMFATLQRSSLWCLCS, |
| | YMFLKAESKIMFATLQRSSLWCLCSN, MFLKAESKIMFATLQRSSLWCLCSNH |
| 27 mers | EPCSMLTGPPARVPAVPFDLHFCRSSI, PCSMLTGPPARVPAVPFDLHFCRSSIM, |
| | CSMLTGPPARVPAVPFDLHFCRSSIMK, SMLTGPPARVPAVPFDLHFCRSSIMKP, |
| | MLTGPPARVPAVPFDLHFCRSSIMKPK, LTGPPARVPAVPFDLHFCRSSIMKPKR, |
| | TGPPARVPAVPFDLHFCRSSIMKPKRD, GPPARVPAVPFDLHFCRSSIMKPKRDG, |
| | PPARVPAVPFDLHFCRSSIMKPKRDGY, PARVPAVPFDLHFCRSSIMKPKRDGYM, |
| | ARVPAVPFDLHFCRSSIMKPKRDGYMF, RVPAVPFDLHFCRSSIMKPKRDGYMFL, |
| | VPAVPFDLHFCRSSIMKPKRDGYMFLK, PAVPFDLHFCRSSIMKPKRDGYMFLKA, |
| | AVPFDLHFCRSSIMKPKRDGYMFLKAE, VPFDLHFCRSSIMKPKRDGYMFLKAES, |
| | PFDLHFCRSSIMKPKRDGYMFLKAESK, FDLHFCRSSIMKPKRDGYMFLKAESKI, |
| | DLHFCRSSIMKPKRDGYMFLKAESKIM, LHFCRSSIMKPKRDGYMFLKAESKIMF, |
| | HFCRSSIMKPKRDGYMFLKAESKIMFA, FCRSSIMKPKRDGYMFLKAESKIMFAT, |
| | CRSSIMKPKRDGYMFLKAESKIMFATL, RSSIMKPKRDGYMFLKAESKIMFATLQ, |
| | SSIMKPKRDGYMFLKAESKIMFATLQR, SIMKPKRDGYMFLKAESKIMFATLQRS, |
| | IMKPKRDGYMFLKAESKIMFATLQRSS, MKPKRDGYMFLKAESKIMFATLQRSSL, |
| | KPKRDGYMFLKAESKIMFATLQRSSLW, PKRDGYMFLKAESKIMFATLQRSSLWC, |
| | KRDGYMFLKAESKIMFATLQRSSLWCL, RDGYMFLKAESKIMFATLQRSSLWCLC, |
| | DGYMFLKAESKIMFATLQRSSLWCLCS, GYMFLKAESKIMFATLQRSSLWCLCSN, |
| | YMFLKAESKIMFATLQRSSLWCLCSNH |
| 28 mers | EPCSMLTGPPARVPAVPFDLHFCRSSIM, PCSMLTGPPARVPAVPFDLHFCRSSIMK, |
| | CSMLTGPPARVPAVPFDLHFCRSSIMKP, SMLTGPPARVPAVPFDLHFCRSSIMKPK, |
| | MLTGPPARVPAVPFDLHFCRSSIMKPKR, LTGPPARVPAVPFDLHFCRSSIMKPKRD, |
| | TGPPARVPAVPFDLHFCRSSIMKPKRDG, GPPARVPAVPFDLHFCRSSIMKPKRDGY, |
| | PPARVPAVPFDLHFCRSSIMKPKRDGYM, PARVPAVPFDLHFCRSSIMKPKRDGYMF, |
| | ARVPAVPFDLHFCRSSIMKPKRDGYMFL, RVPAVPFDLHFCRSSIMKPKRDGYMFLK, |
| | VPAVPFDLHFCRSSIMKPKRDGYMFLKA, PAVPFDLHFCRSSIMKPKRDGYMFLKAE, |
| | AVPFDLHFCRSSIMKPKRDGYMFLKAES, VPFDLHFCRSSIMKPKRDGYMFLKAESK, |
| | PFDLHFCRSSIMKPKRDGYMFLKAESKI, FDLHFCRSSIMKPKRDGYMFLKAESKIM, |
| | DLHFCRSSIMKPKRDGYMFLKAESKIMF, LHFCRSSIMKPKRDGYMFLKAESKIMFA, |
| | HFCRSSIMKPKRDGYMFLKAESKIMFAT, FCRSSIMKPKRDGYMFLKAESKIMFATL, |
| | CRSSIMKPKRDGYMFLKAESKIMFATLQ, RSSIMKPKRDGYMFLKAESKIMFATLQR, |
| | SSIMKPKRDGYMFLKAESKIMFATLQRS, SIMKPKRDGYMFLKAESKIMFATLQRSS, |
| | IMKPKRDGYMFLKAESKIMFATLQRSSL, MKPKRDGYMFLKAESKIMFATLQRSSLW, |
| | KPKRDGYMFLKAESKIMFATLQRSSLWC, PKRDGYMFLKAESKIMFATLQRSSLWCL, |
| | KRDGYMFLKAESKIMFATLQRSSLWCLC, RDGYMFLKAESKIMFATLQRSSLWCLCS, |
| | DGYMFLKAESKIMFATLQRSSLWCLCSN, GYMFLKAESKIMFATLQRSSLWCLCSNH |
| 29 mers | EPCSMLTGPPARVPAVPFDLHFCRSSIMK, PCSMLTGPPARVPAVPFDLHFCRSSIMKP, |
| | CSMLTGPPARVPAVPFDLHFCRSSIMKPK, SMLTGPPARVPAVPFDLHFCRSSIMKPKR, |
| | MLTGPPARVPAVPFDLHFCRSSIMKPKRD, LTGPPARVPAVPFDLHFCRSSIMKPKRDG, |
| | TGPPARVPAVPFDLHFCRSSIMKPKRDGY, GPPARVPAVPFDLHFCRSSIMKPKRDGYM, |
| | PPARVPAVPFDLHFCRSSIMKPKRDGYMF, PARVPAVPFDLHFCRSSIMKPKRDGYMFL, |
| | ARVPAVPFDLHFCRSSIMKPKRDGYMFLK, RVPAVPFDLHFCRSSIMKPKRDGYMFLKA, |
| | VPAVPFDLHFCRSSIMKPKRDGYMFLKAE, PAVPFDLHFCRSSIMKPKRDGYMFLKAES, |
| | AVPFDLHFCRSSIMKPKRDGYMFLKAESK, VPFDLHFCRSSIMKPKRDGYMFLKAESKI, |
| | PFDLHFCRSSIMKPKRDGYMFLKAESKIM, FDLHFCRSSIMKPKRDGYMFLKAESKIMF, |
| | DLHFCRSSIMKPKRDGYMFLKAESKIMFA, LHFCRSSIMKPKRDGYMFLKAESKIMFAT, |
| | HFCRSSIMKPKRDGYMFLKAESKIMFATL, FCRSSIMKPKRDGYMFLKAESKIMFATLQ, |
| | CRSSIMKPKRDGYMFLKAESKIMFATLQR, RSSIMKPKRDGYMFLKAESKIMFATLQRS, |
| | SSIMKPKRDGYMFLKAESKIMFATLQRSS, SIMKPKRDGYMFLKAESKIMFATLQRSSL, |
| | IMKPKRDGYMFLKAESKIMFATLQRSSLW, MKPKRDGYMFLKAESKIMFATLQRSSLWC, |
| | KPKRDGYMFLKAESKIMFATLQRSSLWCL, PKRDGYMFLKAESKIMFATLQRSSLWCLC, |
| | KRDGYMFLKAESKIMFATLQRSSLWCLCS, RDGYMFLKAESKIMFATLQRSSLWCLCSN, |
| | DGYMFLKAESKIMFATLQRSSLWCLCSNH |
| 30 mers | EPCSMLTGPPARVPAVPFDLHFCRSSIMKP, PCSMLTGPPARVPAVPFDLHFCRSSIMKPK, |
| | CSMLTGPPARVPAVPFDLHFCRSSIMKPKR, SMLTGPPARVPAVPFDLHFCRSSIMKPKRD, |
| | MLTGPPARVPAVPFDLHFCRSSIMKPKRDG, LTGPPARVPAVPFDLHFCRSSIMKPKRDGY, |
| | TGPPARVPAVPFDLHFCRSSIMKPKRDGYM, GPPARVPAVPFDLHFCRSSIMKPKRDGYMF, |
| | PPARVPAVPFDLHFCRSSIMKPKRDGYMFL, PARVPAVPFDLHFCRSSIMKPKRDGYMFLK, |
| | ARVPAVPFDLHFCRSSIMKPKRDGYMFLKA, RVPAVPFDLHFCRSSIMKPKRDGYMFLKAE, |
| | VPAVPFDLHFCRSSIMKPKRDGYMFLKAES, PAVPFDLHFCRSSIMKPKRDGYMFLKAESK, |
| | AVPFDLHFCRSSIMKPKRDGYMFLKAESKI, VPFDLHFCRSSIMKPKRDGYMFLKAESKIM, |
| | PFDLHFCRSSIMKPKRDGYMFLKAESKIMF, FDLHFCRSSIMKPKRDGYMFLKAESKIMFA, |
| | DLHFCRSSIMKPKRDGYMFLKAESKIMFAT, LHFCRSSIMKPKRDGYMFLKAESKIMFATL, |
| | HFCRSSIMKPKRDGYMFLKAESKIMFATLQ, FCRSSIMKPKRDGYMFLKAESKIMFATLQR, |
| | CRSSIMKPKRDGYMFLKAESKIMFATLQRS, RSSIMKPKRDGYMFLKAESKIMFATLQRSS, |
| | SSIMKPKRDGYMFLKAESKIMFATLQRSSL, SIMKPKRDGYMFLKAESKIMFATLQRSSLW, |
| | IMKPKRDGYMFLKAESKIMFATLQRSSLWC, |
| | MKPKRDGYMFLKAESKIMFATLQRSSLWCL, |
| | KPKRDGYMFLKAESKIMFATLQRSSLWCLC, PKRDGYMFLKAESKIMFATLQRSSLWCLCS, |
| | KRDGYMFLKAESKIMFATLQRSSLWCLCSN, RDGYMFLKAESKIMFATLQRSSLWCLCSNH |
| 31 mers | EPCSMLTGPPARVPAVPFDLHFCRSSIMKPK, PCSMLTGPPARVPAVPFDLHFCRSSIMKPKR, |
| | CSMLTGPPARVPAVPFDLHFCRSSIMKPKRD, |
| | SMLTGPPARVPAVPFDLHFCRSSIMKPKRDG, |
| | MLTGPPARVPAVPFDLHFCRSSIMKPKRDGY, |
| | LTGPPARVPAVPFDLHFCRSSIMKPKRDGYM, |
| | TGPPARVPAVPFDLHFCRSSIMKPKRDGYMF, |
| | GPPARVPAVPFDLHFCRSSIMKPKRDGYMFL, |
| | PPARVPAVPFDLHFCRSSIMKPKRDGYMFLK, |
| | PARVPAVPFDLHFCRSSIMKPKRDGYMFLKA, |
| | ARVPAVPFDLHFCRSSIMKPKRDGYMFLKAE, |
| | RVPAVPFDLHFCRSSIMKPKRDGYMFLKAES, |
| | VPAVPFDLHFCRSSIMKPKRDGYMFLKAESK, |
| | PAVPFDLHFCRSSIMKPKRDGYMFLKAESKI, |
| | AVPFDLHFCRSSIMKPKRDGYMFLKAESKIM, |
| | VPFDLHFCRSSIMKPKRDGYMFLKAESKIMF, |
| | PFDLHFCRSSIMKPKRDGYMFLKAESKIMFA, |
| | FDLHFCRSSIMKPKRDGYMFLKAESKIMFAT, |
| | DLHFCRSSIMKPKRDGYMFLKAESKIMFATL, |
| | LHFCRSSIMKPKRDGYMFLKAESKIMFATLQ, |
| | HFCRSSIMKPKRDGYMFLKAESKIMFATLQR, |
| | FCRSSIMKPKRDGYMFLKAESKIMFATLQRS, |
| | CRSSIMKPKRDGYMFLKAESKIMFATLQRSS, |
| | RSSIMKPKRDGYMFLKAESKIMFATLQRSSL, |
| | SSIMKPKRDGYMFLKAESKIMFATLQRSSLW, |
| | SIMKPKRDGYMFLKAESKIMFATLQRSSLWC, |
| | IMKPKRDGYMFLKAESKIMFATLQRSSLWCL, |
| | MKPKRDGYMFLKAESKIMFATLQRSSLWCLC, |
| | KPKRDGYMFLKAESKIMFATLQRSSLWCLCS, |
| | PKRDGYMFLKAESKIMFATLQRSSLWCLCSN, |
| | KRDGYMFLKAESKIMFATLQRSSLWCLCSNH |
| 32 mers | EPCSMLTGPPARVPAVPFDLHFCRSSIMKPKR, |
| | PCSMLTGPPARVPAVPFDLHFCRSSIMKPKRD, |
| | CSMLTGPPARVPAVPFDLHFCRSSIMKPKRDG, |
| | SMLTGPPARVPAVPFDLHFCRSSIMKPKRDGY, |
| | MLTGPPARVPAVPFDLHFCRSSIMKPKRDGYM, |
| | LTGPPARVPAVPFDLHFCRSSIMKPKRDGYMF, |
| | TGPPARVPAVPFDLHFCRSSIMKPKRDGYMFL, |
| | GPPARVPAVPFDLHFCRSSIMKPKRDGYMFLK, |
| | PPARVPAVPFDLHFCRSSIMKPKRDGYMFLKA, |
| | PARVPAVPFDLHFCRSSIMKPKRDGYMFLKAE, |
| | ARVPAVPFDLHFCRSSIMKPKRDGYMFLKAES, |
| | RVPAVPFDLHFCRSSIMKPKRDGYMFLKAESK, |
| | VPAVPFDLHFCRSSIMKPKRDGYMFLKAESKI, |
| | PAVPFDLHFCRSSIMKPKRDGYMFLKAESKIM, |
| | AVPFDLHFCRSSIMKPKRDGYMFLKAESKIMF, |
| | VPFDLHFCRSSIMKPKRDGYMFLKAESKIMFA, |
| | PFDLHFCRSSIMKPKRDGYMFLKAESKIMFAT, |
| | FDLHFCRSSIMKPKRDGYMFLKAESKIMFATL, |
| | DLHFCRSSIMKPKRDGYMFLKAESKIMFATLQ, |
| | LHFCRSSIMKPKRDGYMFLKAESKIMFATLQR, |
| | HFCRSSIMKPKRDGYMFLKAESKIMFATLQRS, |
| | FCRSSIMKPKRDGYMFLKAESKIMFATLQRSS, |
| | CRSSIMKPKRDGYMFLKAESKIMFATLQRSSL, |
| | RSSIMKPKRDGYMFLKAESKIMFATLQRSSLW, |
| | SSIMKPKRDGYMFLKAESKIMFATLQRSSLWC, |
| | SIMKPKRDGYMFLKAESKIMFATLQRSSLWCL, |
| | IMKPKRDGYMFLKAESKIMFATLQRSSLWCLC, |
| | MKPKRDGYMFLKAESKIMFATLQRSSLWCLCS, |
| | KPKRDGYMFLKAESKIMFATLQRSSLWCLCSN, |
| | PKRDGYMFLKAESKIMFATLQRSSLWCLCSNH |
| 33 mers | EPCSMLTGPPARVPAVPFDLHFCRSSIMKPKRD, |
| | PCSMLTGPPARVPAVPFDLHFCRSSIMKPKRDG, |
| | CSMLTGPPARVPAVPFDLHFCRSSIMKPKRDGY, |
| | SMLTGPPARVPAVPFDLHFCRSSIMKPKRDGYM, |
| | MLTGPPARVPAVPFDLHFCRSSIMKPKRDGYMF, |
| | LTGPPARVPAVPFDLHFCRSSIMKPKRDGYMFL, |
| | TGPPARVPAVPFDLHFCRSSIMKPKRDGYMFLK, |
| | GPPARVPAVPFDLHFCRSSIMKPKRDGYMFLKA, |
| | PPARVPAVPFDLHFCRSSIMKPKRDGYMFLKAE, |
| | PARVPAVPFDLHFCRSSIMKPKRDGYMFLKAES, |
| | ARVPAVPFDLHFCRSSIMKPKRDGYMFLKAESK, |
| | RVPAVPFDLHFCRSSIMKPKRDGYMFLKAESKI, |
| | VPAVPFDLHFCRSSIMKPKRDGYMFLKAESKIM, |
| | PAVPFDLHFCRSSIMKPKRDGYMFLKAESKIMF, |
| | AVPFDLHFCRSSIMKPKRDGYMFLKAESKIMFA, |
| | VPFDLHFCRSSIMKPKRDGYMFLKAESKIMFAT, |
| | PFDLHFCRSSIMKPKRDGYMFLKAESKIMFATL, |
| | FDLHFCRSSIMKPKRDGYMFLKAESKIMFATLQ, |
| | DLHFCRSSIMKPKRDGYMFLKAESKIMFATLQR, |
| | LHFCRSSIMKPKRDGYMFLKAESKIMFATLQRS, |
| | HFCRSSIMKPKRDGYMFLKAESKIMFATLQRSS, |
| | FCRSSIMKPKRDGYMFLKAESKIMFATLQRSSL, |
| | CRSSIMKPKRDGYMFLKAESKIMFATLQRSSLW, |
| | RSSIMKPKRDGYMFLKAESKIMFATLQRSSLWC, |
| | SSIMKPKRDGYMFLKAESKIMFATLQRSSLWCL, |
| | SIMKPKRDGYMFLKAESKIMFATLQRSSLWCLC, |
| | IMKPKRDGYMFLKAESKIMFATLQRSSLWCLCS, |
| | MKPKRDGYMFLKAESKIMFATLQRSSLWCLCSN, |
| | KPKRDGYMFLKAESKIMFATLQRSSLWCLCSNH |
| 34 mers | EPCSMLTGPPARVPAVPFDLHFCRSSIMKPKRDG, |
| | PCSMLTGPPARVPAVPFDLHFCRSSIMKPKRDGY, |
| | CSMLTGPPARVPAVPFDLHFCRSSIMKPKRDGYM, |
| | SMLTGPPARVPAVPFDLHFCRSSIMKPKRDGYMF, |
| | MLTGPPARVPAVPFDLHFCRSSIMKPKRDGYMFL, |
| | LTGPPARVPAVPFDLHFCRSSIMKPKRDGYMFLK, |
| | TGPPARVPAVPFDLHFCRSSIMKPKRDGYMFLKA, |
| | GPPARVPAVPFDLHFCRSSIMKPKRDGYMFLKAE, |
| | PPARVPAVPFDLHFCRSSIMKPKRDGYMFLKAES, |
| | PARVPAVPFDLHFCRSSIMKPKRDGYMFLKAESK, |
| | ARVPAVPFDLHFCRSSIMKPKRDGYMFLKAESKI, |
| | RVPAVPFDLHFCRSSIMKPKRDGYMFLKAESKIM, |
| | VPAVPFDLHFCRSSIMKPKRDGYMFLKAESKIMF, |
| | PAVPFDLHFCRSSIMKPKRDGYMFLKAESKIMFA, |
| | AVPFDLHFCRSSIMKPKRDGYMFLKAESKIMFAT, |
| | VPFDLHFCRSSIMKPKRDGYMFLKAESKIMFATL, |
| | PFDLHFCRSSIMKPKRDGYMFLKAESKIMFATLQ, |
| | FDLHFCRSSIMKPKRDGYMFLKAESKIMFATLQR, |
| | DLHFCRSSIMKPKRDGYMFLKAESKIMFATLQRS, |
| | LHFCRSSIMKPKRDGYMFLKAESKIMFATLQRSS, |
| | HFCRSSIMKPKRDGYMFLKAESKIMFATLQRSSL, |
| | FCRSSIMKPKRDGYMFLKAESKIMFATLQRSSLW, |
| | CRSSIMKPKRDGYMFLKAESKIMFATLQRSSLWC, |
| | RSSIMKPKRDGYMFLKAESKIMFATLQRSSLWCL, |
| | SSIMKPKRDGYMFLKAESKIMFATLQRSSLWCLC, |
| | SIMKPKRDGYMFLKAESKIMFATLQRSSLWCLCS, |
| | IMKPKRDGYMFLKAESKIMFATLQRSSLWCLCSN, |
| | MKPKRDGYMFLKAESKIMFATLQRSSLWCLCSNH |
| 35 mers | EPCSMLTGPPARVPAVPFDLHFCRSSIMKPKRDGY, |
| | PCSMLTGPPARVPAVPFDLHFCRSSIMKPKRDGYM, |
| | CSMLTGPPARVPAVPFDLHFCRSSIMKPKRDGYMF, |
| | SMLTGPPARVPAVPFDLHFCRSSIMKPKRDGYMFL, |
| | MLTGPPARVPAVPFDLHFCRSSIMKPKRDGYMFLK, |
| | LTGPPARVPAVPFDLHFCRSSIMKPKRDGYMFLKA, |
| | TGPPARVPAVPFDLHFCRSSIMKPKRDGYMFLKAE, |
| | GPPARVPAVPFDLHFCRSSIMKPKRDGYMFLKAES, |
| | PPARVPAVPFDLHFCRSSIMKPKRDGYMFLKAESK, |
| | PARVPAVPFDLHFCRSSIMKPKRDGYMFLKAESKI, |
| | ARVPAVPFDLHFCRSSIMKPKRDGYMFLKAESKIM, |
| | RVPAVPFDLHFCRSSIMKPKRDGYMFLKAESKIMF, |
| | VPAVPFDLHFCRSSIMKPKRDGYMFLKAESKIMFA, |
| | PAVPFDLHFCRSSIMKPKRDGYMFLKAESKIMFAT, |
| | AVPFDLHFCRSSIMKPKRDGYMFLKAESKIMFATL, |
| | VPFDLHFCRSSIMKPKRDGYMFLKAESKIMFATLQ, |
| | PFDLHFCRSSIMKPKRDGYMFLKAESKIMFATLQR, |
| | FDLHFCRSSIMKPKRDGYMFLKAESKIMFATLQRS, |
| | DLHFCRSSIMKPKRDGYMFLKAESKIMFATLQRSS, |
| | LHFCRSSIMKPKRDGYMFLKAESKIMFATLQRSSL, |
| | HFCRSSIMKPKRDGYMFLKAESKIMFATLQRSSLW, |
| | FCRSSIMKPKRDGYMFLKAESKIMFATLQRSSLWC, |
| | CRSSIMKPKRDGYMFLKAESKIMFATLQRSSLWCL, |
| | RSSIMKPKRDGYMFLKAESKIMFATLQRSSLWCLC, |
| | SSIMKPKRDGYMFLKAESKIMFATLQRSSLWCLCS, |
| | SIMKPKRDGYMFLKAESKIMFATLQRSSLWCLCSN, |
| | IMKPKRDGYMFLKAESKIMFATLQRSSLWCLCSNH |
| 36 mers | EPCSMLTGPPARVPAVPFDLHFCRSSIMKPKRDGYM, |
| | PCSMLTGPPARVPAVPFDLHFCRSSIMKPKRDGYMF, |
| | CSMLTGPPARVPAVPFDLHFCRSSIMKPKRDGYMFL, |
| | SMLTGPPARVPAVPFDLHFCRSSIMKPKRDGYMFLK, |
| | MLTGPPARVPAVPFDLHFCRSSIMKPKRDGYMFLKA, |
| | LTGPPARVPAVPFDLHFCRSSIMKPKRDGYMFLKAE, |
| | TGPPARVPAVPFDLHFCRSSIMKPKRDGYMFLKAES, |
| | GPPARVPAVPFDLHFCRSSIMKPKRDGYMFLKAESK, |
| | PPARVPAVPFDLHFCRSSIMKPKRDGYMFLKAESKI, |
| | PARVPAVPFDLHFCRSSIMKPKRDGYMFLKAESKIM, |
| | ARVPAVPFDLHFCRSSIMKPKRDGYMFLKAESKIMF, |
| | RVPAVPFDLHFCRSSIMKPKRDGYMFLKAESKIMFA, |
| | VPAVPFDLHFCRSSIMKPKRDGYMFLKAESKIMFAT, |
| | PAVPFDLHFCRSSIMKPKRDGYMFLKAESKIMFATL, |
| | AVPFDLHFCRSSIMKPKRDGYMFLKAESKIMFATLQ, |
| | VPFDLHFCRSSIMKPKRDGYMFLKAESKIMFATLQR, |
| | PFDLHFCRSSIMKPKRDGYMFLKAESKIMFATLQRS, |
| | FDLHFCRSSIMKPKRDGYMFLKAESKIMFATLQRSS, |
| | DLHFCRSSIMKPKRDGYMFLKAESKIMFATLQRSSL, |
| | LHFCRSSIMKPKRDGYMFLKAESKIMFATLQRSSLW, |
| | HFCRSSIMKPKRDGYMFLKAESKIMFATLQRSSLWC, |
| | FCRSSIMKPKRDGYMFLKAESKIMFATLQRSSLWCL, |
| | CRSSIMKPKRDGYMFLKAESKIMFATLQRSSLWCLC, |
| | RSSIMKPKRDGYMFLKAESKIMFATLQRSSLWCLCS, |
| | SSIMKPKRDGYMFLKAESKIMFATLQRSSLWCLCSN, |
| | SIMKPKRDGYMFLKAESKIMFATLQRSSLWCLCSNH |
| 37 mers | EPCSMLTGPPARVPAVPFDLHFCRSSIMKPKRDGYMF, |
| | PCSMLTGPPARVPAVPFDLHFCRSSIMKPKRDGYMFL, |
| | CSMLTGPPARVPAVPFDLHFCRSSIMKPKRDGYMFLK, |
| | SMLTGPPARVPAVPFDLHFCRSSIMKPKRDGYMFLKA, |
| | MLTGPPARVPAVPFDLHFCRSSIMKPKRDGYMFLKAE, |
| | LTGPPARVPAVPFDLHFCRSSIMKPKRDGYMFLKAES, |
| | TGPPARVPAVPFDLHFCRSSIMKPKRDGYMFLKAESK, |
| | GPPARVPAVPFDLHFCRSSIMKPKRDGYMFLKAESKI, |
| | PPARVPAVPFDLHFCRSSIMKPKRDGYMFLKAESKIM, |
| | PARVPAVPFDLHFCRSSIMKPKRDGYMFLKAESKIMF, |
| | ARVPAVPFDLHFCRSSIMKPKRDGYMFLKAESKIMFA, |
| | RVPAVPFDLHFCRSSIMKPKRDGYMFLKAESKIMFAT, |
| | VPAVPFDLHFCRSSIMKPKRDGYMFLKAESKIMFATL, |
| | PAVPFDLHFCRSSIMKPKRDGYMFLKAESKIMFATLQ, |
| | AVPFDLHFCRSSIMKPKRDGYMFLKAESKIMFATLQR, |
| | VPFDLHFCRSSIMKPKRDGYMFLKAESKIMFATLQRS, |
| | PFDLHFCRSSIMKPKRDGYMFLKAESKIMFATLQRSS, |
| | FDLHFCRSSIMKPKRDGYMFLKAESKIMFATLQRSSL, |
| | DLHFCRSSIMKPKRDGYMFLKAESKIMFATLQRSSLW, |
| | LHFCRSSIMKPKRDGYMFLKAESKIMFATLQRSSLWC, |
| | HFCRSSIMKPKRDGYMFLKAESKIMFATLQRSSLWCL, |
| | FCRSSIMKPKRDGYMFLKAESKIMFATLQRSSLWCLC, |
| | CRSSIMKPKRDGYMFLKAESKIMFATLQRSSLWCLCS, |
| | RSSIMKPKRDGYMFLKAESKIMFATLQRSSLWCLCSN, |
| | SSIMKPKRDGYMFLKAESKIMFATLQRSSLWCLCSNH |
| 38 mers | EPCSMLTGPPARVPAVPFDLHFCRSSIMKPKRDGYMFL, |
| | PCSMLTGPPARVPAVPFDLHFCRSSIMKPKRDGYMFLK, |
| | CSMLTGPPARVPAVPFDLHFCRSSIMKPKRDGYMFLKA, |
| | SMLTGPPARVPAVPFDLHFCRSSIMKPKRDGYMFLKAE, |
| | MLTGPPARVPAVPFDLHFCRSSIMKPKRDGYMFLKAES, |
| | LTGPPARVPAVPFDLHFCRSSIMKPKRDGYMFLKAESK, |
| | TGPPARVPAVPFDLHFCRSSIMKPKRDGYMFLKAESKI, |
| | GPPARVPAVPFDLHFCRSSIMKPKRDGYMFLKAESKIM, |
| | PPARVPAVPFDLHFCRSSIMKPKRDGYMFLKAESKIMF, |
| | PARVPAVPFDLHFCRSSIMKPKRDGYMFLKAESKIMFA, |
| | ARVPAVPFDLHFCRSSIMKPKRDGYMFLKAESKIMFAT, |
| | RVPAVPFDLHFCRSSIMKPKRDGYMFLKAESKIMFATL, |
| | VPAVPFDLHFCRSSIMKPKRDGYMFLKAESKIMFATLQ, |
| | PAVPFDLHFCRSSIMKPKRDGYMFLKAESKIMFATLQR, |
| | AVPFDLHFCRSSIMKPKRDGYMFLKAESKIMFATLQRS, |
| | VPFDLHFCRSSIMKPKRDGYMFLKAESKIMFATLQRSS, |
| | PFDLHFCRSSIMKPKRDGYMFLKAESKIMFATLQRSSL, |
| | FDLHFCRSSIMKPKRDGYMFLKAESKIMFATLQRSSLW, |
| | DLHFCRSSIMKPKRDGYMFLKAESKIMFATLQRSSLWC, |
| | LHFCRSSIMKPKRDGYMFLKAESKIMFATLQRSSLWCL, |
| | HFCRSSIMKPKRDGYMFLKAESKIMFATLQRSSLWCLC, |
| | FCRSSIMKPKRDGYMFLKAESKIMFATLQRSSLWCLCS, |
| | CRSSIMKPKRDGYMFLKAESKIMFATLQRSSLWCLCSN, |
| | RSSIMKPKRDGYMFLKAESKIMFATLQRSSLWCLCSNH |
| 39 mers | EPCSMLTGPPARVPAVPFDLHFCRSSIMKPKRDGYMFLK, |
| | PCSMLTGPPARVPAVPFDLHFCRSSIMKPKRDGYMFLKA, |
| | CSMLTGPPARVPAVPFDLHFCRSSIMKPKRDGYMFLKAE, |
| | SMLTGPPARVPAVPFDLHFCRSSIMKPKRDGYMFLKAES, |
| | MLTGPPARVPAVPFDLHFCRSSIMKPKRDGYMFLKAESK, |
| | LTGPPARVPAVPFDLHFCRSSIMKPKRDGYMFLKAESKI, |
| | TGPPARVPAVPFDLHFCRSSIMKPKRDGYMFLKAESKIM, |
| | GPPARVPAVPFDLHFCRSSIMKPKRDGYMFLKAESKIMF, |
| | PPARVPAVPFDLHFCRSSIMKPKRDGYMFLKAESKIMFA, |
| | PARVPAVPFDLHFCRSSIMKPKRDGYMFLKAESKIMFAT, |
| | ARVPAVPFDLHFCRSSIMKPKRDGYMFLKAESKIMFATL, |
| | RVPAVPFDLHFCRSSIMKPKRDGYMFLKAESKIMFATLQ, |
| | VPAVPFDLHFCRSSIMKPKRDGYMFLKAESKIMFATLQR, |
| | PAVPFDLHFCRSSIMKPKRDGYMFLKAESKIMFATLQRS, |
| | AVPFDLHFCRSSIMKPKRDGYMFLKAESKIMFATLQRSS, |
| | VPFDLHFCRSSIMKPKRDGYMFLKAESKIMFATLQRSSL, |
| | PFDLHFCRSSIMKPKRDGYMFLKAESKIMFATLQRSSLW, |
| | FDLHFCRSSIMKPKRDGYMFLKAESKIMFATLQRSSLWC, |
| | DLHFCRSSIMKPKRDGYMFLKAESKIMFATLQRSSLWCL, |
| | LHFCRSSIMKPKRDGYMFLKAESKIMFATLQRSSLWCLC, |
| | HFCRSSIMKPKRDGYMFLKAESKIMFATLQRSSLWCLCS, |
| | FCRSSIMKPKRDGYMFLKAESKIMFATLQRSSLWCLCSN, |
| | CRSSIMKPKRDGYMFLKAESKIMFATLQRSSLWCLCSNH |
| 40 mers | EPCSMLTGPPARVPAVPFDLHFCRSSIMKPKRDGYMFLKA, |
| | PCSMLTGPPARVPAVPFDLHFCRSSIMKPKRDGYMFLKAE, |
| | CSMLTGPPARVPAVPFDLHFCRSSIMKPKRDGYMFLKAES, |
| | SMLTGPPARVPAVPFDLHFCRSSIMKPKRDGYMFLKAESK, |
| | MLTGPPARVPAVPFDLHFCRSSIMKPKRDGYMFLKAESKI, |
| | LTGPPARVPAVPFDLHFCRSSIMKPKRDGYMFLKAESKIM, |
| | TGPPARVPAVPFDLHFCRSSIMKPKRDGYMFLKAESKIMF, |
| | GPPARVPAVPFDLHFCRSSIMKPKRDGYMFLKAESKIMFA, |
| | PPARVPAVPFDLHFCRSSIMKPKRDGYMFLKAESKIMFAT, |
| | PARVPAVPFDLHFCRSSIMKPKRDGYMFLKAESKIMFATL, |
| | ARVPAVPFDLHFCRSSIMKPKRDGYMFLKAESKIMFATLQ, |
| | RVPAVPFDLHFCRSSIMKPKRDGYMFLKAESKIMFATLQR, |
| | VPAVPFDLHFCRSSIMKPKRDGYMFLKAESKIMFATLQRS, |
| | PAVPFDLHFCRSSIMKPKRDGYMFLKAESKIMFATLQRSS, |
| | AVPFDLHFCRSSIMKPKRDGYMFLKAESKIMFATLQRSSL, |
| | VPFDLHFCRSSIMKPKRDGYMFLKAESKIMFATLQRSSLW, |
| | PFDLHFCRSSIMKPKRDGYMFLKAESKIMFATLQRSSLWC, |
| | FDLHFCRSSIMKPKRDGYMFLKAESKIMFATLQRSSLWCL, |
| | DLHFCRSSIMKPKRDGYMFLKAESKIMFATLQRSSLWCLC, |
| | LHFCRSSIMKPKRDGYMFLKAESKIMFATLQRSSLWCLCS, |
| | HFCRSSIMKPKRDGYMFLKAESKIMFATLQRSSLWCLCSN, |
| | FCRSSIMKPKRDGYMFLKAESKIMFATLQRSSLWCLCSNH |

### Table 2 Below Lists Exemplary Selected Peptides

TABLE 2

| | Exemplary | Mutation Sequence | | |
|---|---|---|---|---|
| | Protein | Context | Peptides (HLA allele | |
| Gene | Change | FRAMESHIFT¹ | example(s)) | Exemplary Diseases |
| GATA3 | L328fs | AQAKAVCSQESRDVL | CLQCLWALL (A02.01) | Breast Cancer |
| | N334fs | CELSDHHNHTLEEEC | CQWGPCLQCL (A02.01) | |
| | | QWGPCLQCLWALLQ | QWGPCLQCL (A24.02) | |
| | | ASQY* | QWGPCLQCLW (A24.02) | |
| GATA3 | H400fs | PGRPLQTHVLPEPHLA | AIQPVLWTT (A02.01) | Breast Cancer |
| | S408fs | LQPLQPHADHAHADA | ALQPLQPHA (A02.01) | |
| | S408fs | PAIQPVLWTTPPLQHG | DLHFCRSSIM (B08.01) | |
| | S430fs | HRHGLEPCSMLTGPP | EPHLALQPL (B07.02, B08.01) | |
| | H434fs | ARVPAVPFDLHFCRSS | ESKIMFATL (B08.01) | |
| | H435fs | IMKPKRDGYMFLKAE | FATLQRSSL (B07.02, B08.01) | |
| | | SKIMFATLQRSSLWCL | FLKAESKIM (B08.01) | |
| | | CSNH* | FLKAESKIMF (B08.01) | |
| | | | GPPARVPAV (B07.02) | |
| | | | IMKPKRDGYM (B08.01) | |
| | | | KIMFATLQR (A03.01) | |
| | | | KPKRDGYMF (B07.02) | |
| | | | KPKRDGYMFL (B07.02) | |
| | | | LHFCRSSIM (B08.01) | |
| | | | LQHGHRHGL (B08.01) | |
| | | | MFATLQRSSL (B07.02, | |
| | | | B08.01) | |
| | | | MFLKAESKI (A24.02) | |
| | | | MLTGPPARV (A02.01) | |
| | | | QPVLWTTPPL (B07.02) | |
| | | | SMLTGPPARV (A02.01) | |
| | | | TLQRSSLWCL (A02.01) | |
| | | | VLPEPHLAL (A02.01) | |
| | | | VPAVPFDLHF (B07.02) | |
| | | | YMFLKAESK (A03.01) | |
| | | | YMFLKAESKI (A02.01, | |
| | | | A03.01, A24.02, B08.01) | |

¹Underlined AAs represent non-native AAs

TABLE 3

| | | | | |
|---|---|---|---|---|
| | Exemplary | | | |
| | Protein | Mutation Sequence | | |
| Gene | Change | Context | Peptides (HLA allele example(s)) | Exemplary Diseases |

Table 3A

| | | POINT MUTATIONS¹ | | |
|---|---|---|---|---|
| AKT1 | E17K | MSDVAIVKEGWLHKR | KYIKTWRPRY (A24.02) | BRCA, CESC, HNSC, |
| | | GKYIKTWRPRYFLLK | WLHKRGKYI (A02.01, B07.02, | LUSC, PRAD, SKCM, |
| | | NDGTFIGYKERPQDV | B08.01) | THCA |
| | | DQREAPLNNFSVAQC | WLHKRGKYIK (A03.01) | |
| | | QLMKTER | | |
| ANAPC1 | T537A | TMLVLEGSGNLVLYT | APKPLSKLL (B07.02) | GBM, LUSC, PAAD, |
| | | GVVRVGKVFIPGLPAP | GVSAPKPLSK (A03.01) | PRAD, SKCM |
| | | SLTMSNTMPRPSTPLD | VSAPKPLSK (A03.01) | |
| | | GVSAPKPLSKLLGSLD | | |
| | | EVVLLSPVPELRDSSK | | |
| | | LHDSLYNEDCTFQQL | | |
| | | GTYIHSI | | |
| FGFR3 | S249C | HRIGGIKLRHQQWSL | CPHRPILQA (B07.02) | BLCA, HNSC, KIRP, |
| | | VMESVVPSDRGNYTC | | LUSC |
| | | VVENKFGSIRQTYTLD | | |
| | | VLERCPHRPILQAGLP | | |
| | | ANQTAVLGSDVEFHC | | |
| | | KVYSDAQPHIQWLKH | | |
| | | VEVNGSKVG | | |
| FRG1B | I10T | MREPIYMHSTMVFLP | KLSDSRTAL (A02.01, B07.02, | KIRP, PRAD, SKCM |
| | | WELHTKKGPSPPEQF | B08.01) | |
| | | MAVKLSDSRTALKSG | KLSDSRTALK (A03.01) | |
| | | YGKYLGINSDELVGH | LSDSRTALK (A01.01, A03.01) | |
| | | SDAIGPREQWEPVFQ | RTALKSGYGK (A03.01) | |
| | | NGKMALLASNSCFIR | TALKSGYGK (A03.01) | |
| FRG1B | L52S | AVKLSDSRIALKSGYG | ALSASNSCF (A02.01, A24.02, | GBM, KIRP, PRAD, |
| | | KYLGINSDELVGHSD | B07.02) | SKCM |
| | | AIGPREQWEPVFQNG | ALSASNSCFI (A02.01) | |
| | | KMALSASNSCFIRCNE | FQNGKMALSA (A02.01, B08.01) | |
| | | AGDIEAKSKTAGEEE | | |
| | | MIKIRSCAEKETKKKD | | |
| | | DIPEEDKG | | |
| HER2 | L755S | AMPNQAQMRILKETE | KVSRENTSPK (A03.01) | BRCA |
| | (Resistance) | LRKVKVLGSGAFGTV | | |
| | | YKGIWIPDGENVKIPV | | |
| | | AIKVSRENTSPKANKE | | |
| | | ILDEAYVMAGVGSPY | | |
| | | VSRLLGICLTSTVQLV | | |
| | | TQLMPYGC | | |
| IDH1 | R132G | RVEEFKLKQMWKSPN | KPIIIGGHAY (B07.02) | BLCA, BRCA, CRC, |
| | | GTIRNILGGTVFREAII | | GBM, HNSC, LUAD, |
| | | CKNIPRLVSGWVKPIII | | PAAD, PRAD, UCEC |
| | | GGHAYGDQYRATDF | | |
| | | VVPGPGKVEITYTPSD | | |
| | | GTQKVTYLVHNFEEG | | |
| | | GGVAMGM | | |
| KRAS | G12C | MTEYKLVVVGACGV | KLVVVGACGV (A02.01) | BRCA, CESC, CRC, |
| | | GKSALTIQLIQNHFVD | LVVVGACGV (A02.01) | HNSC, LUAD, PAAD, |
| | | EYDPTIEDSYRKQVVI | VVGACGVGK (A03.01, A11.01) | UCEC |
| | | DGETCLLDILDTAGQE | VVVGACGVGK (A03.01) | |
| KRAS | G12D | MTEYKLVVVGADGV | VVGADGVGK (A11.01) | BLCA, BRCA, CESC, |
| | | GKSALTIQLIQNHFVD | VVVGADGVGK (A11.01) | CRC, GBM, HNSC, |
| | | EYDPTIEDSYRKQVVI | KLVVVGADGV (A02.01) | KIRP, LIHC, LUAD, |
| | | DGETCLLDILDTAGQE | LVVVGADGV (A02.01) | PAAD, SKCM, UCEC |
| KRAS | G12V | MTEYKLVVVGAVGV | KLVVVGAVGV (A02.01) | BRCA, CESC, CRC, |
| | | GKSALTIQLIQNHFVD | LVVVGAVGV (A02.01) | LUAD, PAAD, THCA, |
| | | EYDPTIEDSYRKQVVI | VVGAVGVGK (A03.01, A11.01) | UCEC |
| | | DGETCLLDILDTAGQE | VVVGAVGVGK (A03.01, A11.01) | |
| KRAS | Q61H | AGGVGKSALTIQLIQN | ILDTAGHEEY (A01.01) | CRC, LUSC, PAAD, |
| | | HFVDEYDPTIEDSYRK | | SKCM, UCEC |
| | | QVVIDGETCLLDILDT | | |
| | | AGHEEYSAMRDQYM | | |
| | | RTGEGFLCVFAINNTK | | |
| | | SFEDIHHYREQIKRVK | | |
| | | DSEDVPM | | |
| KRAS | Q61L | AGGVGKSALTIQLIQN | ILDTAGLEEY (A01.01) | CRC, GBM, HNSC, |
| | | HFVDEYDPTIEDSYRK | LLDILDTAGL (A02.01) | LUAD, SKCM, UCEC |
| | | QVVIDGETCLLDILDT | | |
| | | AGLEEYSAMRDQYM | | |
| | | RTGEGFLCVFAINNTK | | |
| | | SFEDIHHYREQIKRVK | | |
| | | DSEDVPM | | |
| NRAS | Q61K | AGGVGKSALTIQLIQN | ILDTAGKEEY (A01.01) | BLCA, CRC, LIHC, |
| | | HFVDEYDPTIEDSYRK | | LUAD, LUSC, SKCM, |
| | | QVVIDGETCLLDILDT | | THCA, UCEC |
| | | AGKEEYSAMRDQYM | | |
| | | RTGEGFLCVFAINNSK | | |
| | | SFADINLYREQIKRVK | | |
| | | DSDDVPM | | |
| NRAS | Q61R | AGGVGKSALTIQLIQN | ILDTAGREEY (A01.01) | BLCA, CRC, LUSC, |
| | | HFVDEYDPTIEDSYRK | | PAAD, PRAD, SKCM, |
| | | QVVIDGETCLLDILDT | | THCA, UCEC |
| | | AGREEYSAMRDQYM | | |
| | | RTGEGFLCVFAINNSK | | |
| | | SFADINLYREQIKRVK | | |
| | | DSDDVPM | | |
| PIK3CA | E542K | IEEHANWSVSREAGFS | AISTRDPLSK (A03.01) | BLCA, BRCA, CESC, |
| | | YSHAGLSNRLARDNE | | CRC, GBM, HNSC, |
| | | LRENDKEQLKAISTRD | | KIRC, KIRP, LIHC, |
| | | PLSKITEQEKDFLWSH | | LUAD, LUSC, PRAD, |
| | | RHYCVTIPEILPKLLLS | | UCEC |
| | | VKWNSRDEVAQMYC | | |
| | | LVKDWPP | | |
| PTEN | R130Q | KFNCRVAQYPFEDHN | QTGVMICAY (A01.01) | BRCA, CESC, CRC, |
| | | PPQLELIKPFCEDLDQ | | GBM, KIRC, LUSC, |
| | | WLSEDDNHVAAIHCK | | UCEC |
| | | AGKGQTGVMICAYLL | | |
| | | HRGKFLKAQEALDFY | | |
| | | GEVRTRDKKGVTIPSQ | | |
| | | RRYVYYYSY | | |
| RAC1 | P29S | MQAIKCVVVGDGAV | FSGEYIPTV (A02.01) | Melanoma |
| | | GKTCLLISYTTNAFSG | TTNAFSGEY (A01.01) | |
| | | EYIPTVFDNYSANVM | YTTNAFSGEY (A01.01) | |
| | | VDGKPVNLGLWDTA | | |
| | | GQEDYDRLRPLSYPQ | | |
| | | TVGET | | |
| SF3B1 | K700E | AVCKSKKSWQARHT | GLVDEQQEV (A02.01) | AML associated with |
| | | GIKIVQQIAILMGCAIL | | MDS; Chronic |
| | | PHLRSLVEIIEHGLVD | | lymphocytic leukemia- |
| | | EQQEVRTISALAIAAL | | small lymphocytic |
| | | AEAATPYGIESFDSVL | | lymphoma; |
| | | KPLWKGIRQHRGKGL | | Myelodysplastic |
| | | AAFLKAI | | syndrome; AML; |
| | | | | Luminal NS carcinoma |
| | | | | of breast; Chronic |
| | | | | myeloid leukemia; |
| | | | | Ductal carcinoma of |
| | | | | pancreas; Chronic |
| | | | | myelomonocytic |
| | | | | leukemia; Chronic |
| | | | | lymphocytic leukemia- |
| | | | | small lymphocytic |
| | | | | lymphoma; |
| | | | | Myelofibrosis; |
| | | | | Myelodysplastic |
| | | | | syndrome; PRAD; |
| | | | | Essential |
| | | | | thrombocythaemia; |
| | | | | Medullomyoblastoma |
| SPOP | F133L | YLSLYLLLVSCPKSEV | FVQGKDWGL (A02.01, B08.01) | PRAD |
| | | RAKFKFSILNAKGEET | | |
| | | KAMESQRAYRFVQG | | |
| | | KDWGLKKFIRRDFLL | | |
| | | DEANGLLPDDKLTLF | | |
| | | CEVSVVQDSVNISGQ | | |
| | | NTMNMVKVPE | | |
| SPOP | F133V | YLSLYLLLVSCPKSEV | FVQGKDWGV (A02.01) | PRAD |
| | | RAKFKFSILNAKGEET | | |
| | | KAMESQRAYRFVQG | | |
| | | KDWGVKKFIRRDFLL | | |
| | | DEANGLLPDDKLTLF | | |
| | | CEVSVVQDSVNISGQ | | |
| | | NTMNMVKVPE | | |
| TP53 | G245S | IRVEGNLRVEYLDDR | CMGSMNRRPI (A02.01, B08.01) | BLCA, BRCA, CRC, |
| | | NTFRHSVVVPYEPPEV | GSMNRRPIL (B08.01) | GBM, HNSC, LUSC, |
| | | GSDCTTIHYNYMCNS | MGSMNRRPI (B08.01) | PAAD, PRAD |
| | | SCMGSMNRRPILTIITL | MGSMNRRPIL (B08.01) | |
| | | EDSSGNLLGRNSFEVR | SMNRRPILTI (A02.01, A24.02, | |
| | | VCACPGRDRRTEEEN | B08.01) | |
| | | LRKKGEP | | |
| TP53 | R248Q | EGNLRVEYLDDRNTF | CMGGMNQRPI (A02.01, B08.01) | BLCA, BRCA, CRC, |
| | | RHSVVVPYEPPEVGS | GMNQRPILTI (A02.01, B08.01) | GBM, HNSC, KIRC, |
| | | DCTTIHYNYMCNSSC | NQRPILTII (A02.01, B08.01) | LIHC, LUSC, PAAD, |
| | | MGGMNQRPILTIITLE | | PRAD, UCEC |
| | | DSSGNLLGRNSFEVR | | |
| | | VCACPGRDRRTEEEN | | |
| | | LRKKGEPHHE | | |
| TP53 | R248W | EGNLRVEYLDDRNTF | CMGGMNWRPI (A02.01, A24.02, | BLCA, BRCA, CRC, |
| | | RHSVVVPYEPPEVGS | B08.01) | GBM, HNSC, LIHC, |
| | | DCTTIHYNYMCNSSC | GMNWRPILTI (A02.01, B08.01) | LUSC, PAAD, SKCM, |
| | | MGGMNWRPILTIITLE | MNWRPILTI (A02.01, A24.02, | UCEC |
| | | DSSGNLLGRNSFEVR | B08.01) | |
| | | VCACPGRDRRTEEEN | MNWRPILTII (A02.01, A24.02) | |
| | | LRKKGEPHHE | | |
| TP53 | R273C | PEVGSDCTTIHYNYM | NSFEVCVCA (A02.01) | BLCA, BRCA, CRC, |
| | | CNSSCMGGMNRRPIL | | GBM, HNSC, LUSC, |
| | | TIITLEDSSGNLLGRNS | | PAAD, UCEC |
| | | FEVCVCACPGRDRRT | | |
| | | EEENLRKKGEPHHELP | | |
| | | PGSTKRALPNNTSSSP | | |
| | | QPKKKPL | | |
| TP53 | R273H | PEVGSDCTTIHYNYM | NSFEVHVCA (A02.01) | BRCA, CRC, GBM, |
| | | CNSSCMGGMNRRPIL | | HNSC, LIHC, LUSC, |
| | | TIITLEDSSGNLLGRNS | | PAAD, UCEC |
| | | FEVHVCACPGRDRRT | | |
| | | EEENLRKKGEPHHELP | | |
| | | PGSTKRALPNNTSSSP | | |
| | | QPKKKPL | | |
| TP53 | Y220C | TEVVRRCPHHERCSD | VVPCEPPEV (A02.01) | BLCA, BRCA, GBM, |
| | | SDGLAPPQHLIRVEGN | VVVPCEPPEV (A02.01) | HNSC, LIHC, LUAD, |
| | | LRVEYLDDRNTFRHS | | LUSC, PAAD, SKCM, |
| | | VVVPCEPPEVGSDCTT | | UCEC |
| | | IHYNYMCNSSCMGG | | |
| | | MNRRPILTIITLEDSSG | | |
| | | NLLGRNSF | | |

Table 3B

| | | MSI-ASSOCIATED | | |
|---|---|---|---|---|
| | | FRAMESHIFTS¹ | | |
| MSH6 | F1088fs; +1 | YNFDKNYKDWQSAV | ILLPEDTPPL (A02.01) | MSI + CRC, MSI + |
| | | ECIAVLDVLLCLANYS | LLPEDTPPL (A02.01) | Uterine/Endometrium |
| | | RGGDGPMCRPVILLPE | | Cancer, MSI + Stomach |
| | | DTPPLLRA | | Cancer, Lynch |
| | | | | syndrome |

Table 3C

| | | FRAMESHIFT¹ | | |
|---|---|---|---|---|
| APC | F1354fs | AKFQQCHSTLEPNPA | APFRVNHAV (B07.02) | CRC, LUAD, UCEC, |
| | | DCRVLVYLQNQPGTK | CLADVLLSV (A02.01) | STAD |
| | | LLNFLQERNLPPKVVL | FLQERNLPPK (A03.01) | |
| | | RHPKVHLNTMFRRPH | HLIVLRVVRL (A02.01, B08.01) | |
| | | SCLADVLLSVHLIVLR | HPKVHLNTM (B07.02, B08.01) | |
| | | VVRLPAPFRVNHAVE | HPKVHLNTMF (B07.02, B08.01) | |
| | | W* | KVHLNTMFR (A03.01) | |
| | | | KVHLNTMFRR (A03.01) | |
| | | | LPAPFRVNHA (B07.02) | |
| | | | MFRRPHSCL (B07.02, B08.01) | |
| | | | MFRRPHSCLA (B08.01) | |
| | | | NTMFRRPHSC (B08.01) | |
| | | | RPHSCLADV (B07.02) | |
| | | | RPHSCLADVL (B07.02) | |
| | | | RVVRLPAPFR (A03.01) | |
| | | | SVHLIVLRV (A02.01) | |
| | | | TMFRRPHSC (B08.01) | |
| | | | TMFRRPHSCL (A02.01, B08.01) | |
| | | | VLLSVHLIV (A02.01) | |
| | | | VLLSVHLIVL (A02.01) | |
| | | | VLRVVRLPA (B08.01) | |
| | | | VVRLPAPFR (A03.01) | |
| ARID1A | Y1324fs | ALGPHSRISCLPTQTR | AMPILPLPQL (A02.01) | STAD, UCEC, BLCA, |
| | | GCILLAATPRSSSSSSS | APLLAAPSPA (B07.02) | BRCA, LUSC, CESC, |
| | | NDMIPMAISSPPKAPL | APRTNFHSS (B07.02) | KIRC, UCS |
| | | LAAPSPASRLQCINSN | APRTNFHSSL (B07.02, B08.01) | |
| | | SRITSGQWMAHMALL | CPQPSPSLPA (B07.02) | |
| | | PSGTKGRCTACHTAL | GQWMAHMAL (A02.01) | |
| | | GRGSLSSSSCPQPSPSL | GQWMAHMALL (A02.01) | |
| | | PASNKLPSLPLSKMYT | HMALLPSGTK (A03.01) | |
| | | TSMAMPILPLPQLLLS | HTALGRGSL (B07.02) | |
| | | ADQQAAPRTNFHSSL | IPMAISSPP (B07.02) | |
| | | AETVSLHPLAPMPSKT | IPMAISSPPK (B07.02) | |
| | | CHHK* | KLPSLPLSK (A03.01) | |
| | | | KLPSLPLSKM (A02.01) | |
| | | | KMYTTSMAM (A02.01, A03.01) | |
| | | | LLAAPSPASR (A03.01) | |
| | | | LLLSADQQAA (A02.01) | |
| | | | LLSADQQAA (A02.01) | |
| | | | LPASNKLPS (B07.02) | |
| | | | LPASNKLPSL (B07.02, B08.01) | |
| | | | LPLPQLLLSA (B07.02) | |
| | | | LPSLPLSKM (B07.02) | |
| | | | LSKMYTTSM (B08.01) | |
| | | | MALLPSGTK (A03.01) | |
| | | | MPILPLPQL (B07.02) | |
| | | | MPILPLPQLL (B07.02) | |
| | | | MYTTSMAMPI (A24.02) | |
| | | | PMAISSPPK (A03.01) | |
| | | | QWMAHMALL (A24.02) | |
| | | | SKMYTTSMAM (B07.02) | |
| | | | SMAMPILPL (A02.01, B07.02, | |
| | | | B08.01) | |
| | | | SNKLPSLPL (B08.01) | |
| | | | SPASRLQCI (B07.02, B08.01) | |
| | | | SPPKAPLLAA (B07.02) | |
| | | | SPSLPASNKL (B07.02) | |
| | | | YTTSMAMPI (A02.01) | |
| | | | YTTSMAMPIL (A02.01) | |
| ARID1A | G1848fs | RSYRRMIHLWWTAQI | CLPGLTHPA (A02.01) | STAD, UCEC, BLCA, |
| | | SLGVCRSLTVACCTG | GLTHPAHQPL (A02.01) | BRCA, LUSC, CESC, |
| | | GLVGGTPLSISRPTSR | HPAHQPLGSM (B07.02) | KIRC, UCS |
| | | ARQSCCLPGLTHPAH | LTHPAHQPL (B07.02) | |
| | | QPLGSM* | RPTSRARQSC (B07.02) | |
| | | | RQSCCLPGL (A02.01) | |
| | | | TSRARQSCCL (B08.01) | |
| β2M | L13fs | QHSGRDVSLRGLSCA | ELLCVWVSSI (A02.01) | CRC, STAD, SKCM, |
| | | RATLSFWPGGYPAYS | EWKVKFPEL (B08.01) | HNSC |
| | | KDSGLLTSSSREWKV | KFPELLCVW (A24.02) | |
| | | KFPELLCVWVSSIRH* | LLCVWVSSI (A02.01) | |
| | | | LLTSSSREWK (A03.01) | |
| | | | LTSSSREWK (A03.01) | |
| | | | YPAYSKDSGL (B07.02) | |
| GATA3 | L328fs | AQAKAVCSQESRDVL | CLQCLWALL (A02.01) | Breast Cancer |
| | N334fs | CELSDHHNHTLEEEC | CQWGPCLQCL (A02.01) | |
| | | QWGPCLQCLWALLQ | QWGPCLQCL (A24.02) | |
| | | ASQY* | QWGPCLQCLW (A24.02) | |
| GATA3 | H400fs | PGRPLQTHVLPEPHLA | AIQPVLWTT (A02.01) | |
| | S408fs | LQPLQPHADHAHADA | ALQPLQPHA (A02.01) | |
| | S408fs | PAIQPVLWTTPPLQHG | DLHFCRSSIM (B08.01) | |
| | S430fs | HRHGLEPCSMLTGPP | EPHLALQPL (B07.02, B08.01) | |
| | H434fs | ARVPAVPFDLHFCRSS | ESKIMFATL (B08.01) | |
| | H435fs | IMKPKRDGYMFLKAE | FATLQRSSL (B07.02, B08.01) | |
| | | SKIMFATLQRSSLWCL | FLKAESKIM (B08.01) | |
| | | CSNH* | FLKAESKIMF (B08.01) | |
| | | | GPPARVPAV (B07.02) | |
| | | | IMKPKRDGYM (B08.01) | |
| | | | KIMFATLQR (A03.01) | |
| | | | KPKRDGYMF (B07.02) | |
| | | | KPKRDGYMFL (B07.02) | |
| | | | LHFCRSSIM (B08.01) | Breast Cancer |
| | | | LQHGHRHGL (B08.01) | |
| | | | MFATLQRSSL (B07.02, B08.01) | |
| | | | MFLKAESKI (A24.02) | |
| | | | MLTGPPARV (A02.01) | |
| | | | QPVLWTTPPL (B07.02) | |
| | | | SMLTGPPARV (A02.01) | |
| | | | TLQRSSLWCL (A02.01) | |
| | | | VLPEPHLAL (A02.01) | |
| | | | VPAVPFDLHF (B07.02) | |
| | | | YMFLKAESK (A03.01) | |
| | | | YMFLKAESKI (A02.01, A03.01, | |
| | | | A24.02, B08.01) | |
| MLL2 | P647fs | TRRCHCCPHLRSHPCP | APGPRGRTC (B07.02) | STAD, BLCA, CRC, |
| | L656fs | HHLRNHPRPHHLRHH | CLRSHTCPPR (A03.01) | HNSC, BRCA |
| | | ACHHHLRNCPHPHFL | CLWCHACLHR (A03.01) | |
| | | RHCTCPGRWRNRPSL | CPHLGSHPC (B07.02) | |
| | | RRLRSLLCLPHLNHHL | CPLGLKSPL (B07.02) | |
| | | FLHWRSRPCLHRKSH | CPRSCRCPH (B07.02) | |
| | | PHLLHLRRLYPHHLK | CPRSCRCPHL (B07.02, B08.01) | |
| | | HRPCPHHLKNLLCPR | CSLPLGNHPY (A01.01) | |
| | | HLRNCPLPRHLKHLA | GLRNRICPL (A02.01, B07.02, | |
| | | CLHHLRSHPCPLHLKS | B08.01) | |
| | | HPCLHHRRHLVCSHH | GLRSHTYLR (A03.01) | |
| | | LKSLLCPLHLRSLPFP | GLRSHTYLRR (A03.01) | |
| | | HHLRHHACPHHLRTR | GPRGRTCHPG (B07.02) | |
| | | LCPHHLKNHLCPPHL | HLGSHPCRL (B08.01) | |
| | | RYRAYPPCLWCHACL | HLRLHASPH (A03.01) | |
| | | HRLRNLPCPHRLRSLP | HLRSCPCSL (B07.02, B08.01) | |
| | | RPLHLRLHASPHHLRT | HLRTHLLPH (A03.01) | |
| | | PPHPHHLRTHLLPHHR | HLRTHLLPHH (A03.01) | |
| | | RTRSCPCRWRSHPCC | HLRYRAYPP (B08.01) | |
| | | HYLRSRNSAPGPRGR | HLRYRAYPPC (B08.01) | |
| | | TCHPGLRSRTCPPGLR | HPHHLRTHL (B07.02) | |
| | | SHTYLRRLRSHTCPPS | HPHHLRTHLL (B07.02, B08.01) | |
| | | LRSHAYALCLRSHTCP | HTYLRRLRSH (A03.01) | |
| | | PRLRDHICPLSLRNCT | LPCPHRLRSL (B07.02, B08.01) | |
| | | CPPRLRSRTCLLCLRS | LPHHRRTRSC (B07.02, B08.01) | |
| | | HACPPNLRNHTCPPSL | LPLGNHPYL (B07.02) | |
| | | RSHACPPGLRNRICPL | LPRPLHLRL (B07.02, B08.01) | |
| | | SLRSHPCPLGLKSPLR | NLRNHTCPP (B08.01) | |
| | | SQANALHLRSCPCSLP | PPRLRSRTCL (B07.02, B08.01) | |
| | | LGNHPYLPCLESQPCL | RLHASPHHL (A02.01) | |
| | | SLGNHLCPLCPRSCRC | RLHASPHHLR (A03.01) | |
| | | PHLGSHPCRLS* | RLRDHICPL (A02.01, B07.02, | |
| | | | B08.01) | |
| | | | RLRNLPCPH (A03.01) | |
| | | | RLRNLPCPHR (A03.01) | |
| | | | RLRSHTCPP (B08.01) | |
| | | | RLRSLPRPL (B07.02, B08.01) | |
| | | | RLRSLPRPLH (A03.01) | |
| | | | RLRSRTCLL (B07.02, B08.01) | |
| | | | RNRICPLSL (B07.02, B08.01) | |
| | | | RPLHLRLHA (B07.02) | |
| | | | RPLHLRLHAS (B07.02) | |
| | | | RSHACPPGLR (A03.01) | |
| | | | RSHACPPNLR (A03.01) | |
| | | | RSHAYALCLR (A03.01) | |
| | | | RSHPCCHYLR (A03.01) | |
| | | | RSHPCPLGLK (A03.01) | |
| | | | RSHTCPPSLR (A03.01) | |
| | | | RSLPRPLHLR (A03.01) | |
| | | | RSRTCLLCL (B07.02) | |
| | | | RSRTCLLCLR (A03.01) | |
| | | | RSRTCPPGL (B07.02) | |
| | | | RSRTCPPGLR (A03.01) | |
| | | | RTHLLPHHRR (A03.01) | |
| | | | RTRSCPCRWR (A03.01) | |
| | | | RYRAYPPCL (A24.02) | |
| | | | RYRAYPPCLW (A24.02) | |
| | | | SLGNHLCPL (A02.01, B07.02, | |
| | | | B08.01) | |
| | | | SLPLGNHPYL (A02.01) | |
| | | | SLPRPLHLRL (A02.01) | |
| | | | SLRNCTCPPR (A03.01) | |
| | | | SLRSHAYAL (A02.01, B07.02, | |
| | | | B08.01) | |
| | | | SLRSHPCPL (A02.01, B07.02, | |
| | | | B08.01) | |
| | | | SPHHLRTPP (B07.02) | |
| | | | SPHHLRTPPH (B07.02) | |
| | | | SPLRSQANAL (B07.02, B08.01) | |
| | | | YLRRLRSHTC (B08.01) | |
| | | | YLRSRNSAP (B08.01) | |
| | | | YLRSRNSAPG (B08.01) | |
| MLL2 | P2354fs | GPRSHPLPRLWHLLL | ALAPTLTHM (A02.01) | STAD, BLCA, CRC, |
| | | QVTQTSFALAPTLTH | ALAPTLTHML (A02.01) | HNSC, BRCA |
| | | MLSPH* | LLQVTQTSFA (A02.01) | |
| | | | LQVTQTSFAL (A02.01) | |
| | | | RLWHLLLQV (A02.01) | |
| | | | RLWHLLLQVT (A02.01) | |
| RNF43 | G659fs | PLGLVPWTRWCPQGK | CTQLARFFPI (A24.02) | STAD |
| | | PRFPAMSTTTATGTTT | FFPITPPVW (A24.02) | |
| | | TKSGSSGMAGSLAQK | FPITPPVWHI (B07.02) | |
| | | PESPSPGLLFLGHSPSQ | GPRMQLCTQL (B07.02, B08.01) | |
| | | SHLLLISKSPDPTQQPL | ITPPVWHIL (A24.02) | |
| | | RGGSLTHSAPGPSLSQ | LALGPRMQL (B07.02) | |
| | | PLAQLTPPASAPVPAV | MQLCTQLARF (A24.02) | |
| | | CSTCKNPASLPDTHRG | RFFPITPPV (A02.01, A24.02) | |
| | | KGGGVPPSPPLALGPR | RFFPITPPVW (A24.02) | |
| | | MQLCTQLARFFPITPP | RMQLCTQLA (A02.01) | |
| | | VWHILGPQRHTP* | RMQLCTQLAR (A03.01) | |
| | | | SPPLALGPRM (B07.02) | |
| | | | TQLARFFPI (A02.01, A24.02, | |
| | | | B08.01) | |
| SMAP1 | E169fs | KYEKKKYYDKNAIAI | KSRQNHLQL (B07.02) | MSI + CRC, MSI + |
| | | TNISSSDAPLQPLVSSP | ALKKLRSPL (B08.01, B07.02) | Uterine/Endometrium |
| | | SLQAAVDKNKLEKEK | HLQLKSCRRK (A03.01) | Cancer, MSI + Stomach |
| | | EKKRKRKREKRSQKS | KISNWSLKK (A03.01, A11.01) | Cancer |
| | | RQNHLQLKSCRRKISN | KISNWSLKKV (A03.01) | |
| | | WSLKKVPALKKLRSP | KLRSPLWIF (A24.02) | |
| | | LWIF | KSRQNHLQLK (A03.01) | |
| | | | NWSLKKVPAL (B08.01) | |
| | | | SLKKVPALK (A03.01, A11.01) | |
| | | | SLKKVPALKK (A03.01) | |
| | | | SQKSRQNHL (B08.01) | |
| | | | WSLKKVPAL (B08.01) | |
| | | | WSLKKVPALK (A03.01) | |
| TP53 | P58fs | CCPRTILNNGSLKTQV | KLPECQRLL (A02.01) | BRCA, CRC, LUAD, |
| | P72fs | QMKLPECQRLLPPWP | KPTRAATVSV (B07.02) | PRAD, HNSC, LUSC, |
| | G108fs | LHQQLLHRRPLHQPPP | LPPWPLHQQL (B07.02) | PAAD, STAD, BLCA, |
| | R110fs | GPCHLLSLPRKPTRAA | LPRKPTRAA (B07.02, B08.01) | OV, LIHC, SKCM, |
| | | TVSVWASCILGQPSL* | LPRKPTRAAT (B07.02) | UCEC, LAML, UCS, |
| | | | QQLLHRRPL (B08.01) | KICH, GBM, ACC |
| | | | RLLPPWPLH (A03.01) | |
| TP53 | P152fs | LARTPLPSTRCFANWP | APASAPWPST (B07.02) | BRCA, CRC, LUAD, |
| | | RPALCSCGLIPHPRPA | APWPSTSSH (B07.02) | PRAD, HNSC, LUSC, |
| | | PASAPWPSTSSHST* | RPAPASAPW (B07.02) | PAAD, STAD, BLCA, |
| | | | WPSTSSHST (B07.02) | OV, LIHC, SKCM, |
| | | | | UCEC, LAML, UCS, |
| | | | | KICH, GBM, ACC |
| UBR5 | K2120fs | SQGLYSSSASSGKCL | RVQNQGHLL (B07.02) | |
| | | MEVTVDRNCLEVLPT | | |
| | | KMSYAANLKNVMNM | | |
| | | QNRQKKKGKNSPCCQ | | |
| | | KKLRVQNQGHLLMIL | | |
| | | LHN* | | |
| VHL | L116fs | TRASPPRSSSAIAVRA | FLPISHCQCI (A02.01) | KIRC, KIRP |
| | G123fs | SCCPYGSTSTASRSPT | FWLTKLNYL (A24.02, B08.01) | |
| | | QRCRLARAAASTATE | HLSMLTDSL (A02.01) | |
| | | VTFGSSEMQGHTMGF | HTMGFWLTK (A03.01) | |
| | | WLTKLNYLCHLSMLT | HTMGFWLTKL (A02.01) | |
| | | DSLFLPISHCQCIL* | KLNYLCHLSM (A02.01) | |
| | | | LPISHCQCI (B07.02, B08.01) | |
| | | | LPISHCQCIL (B07.02, B08.01) | |
| | | | LTDSLFLPI (A01.01, A02.01) | |
| | | | LTKLNYLCHL (B08.01) | |
| | | | MLTDSLFLPI (A01.01, A02.01, | |
| | | | B08.01) | |
| | | | MQGHTMGFWL (A02.01) | |
| | | | NYLCHLSML (A24.02) | |
| | | | SMLTDSLFL (A02.01) | |
| | | | TMGFWLTKL (A02.01) | |
| | | | YLCHLSMLT (A02.01) | |

TABLE 3D

| | | INSERT¹ | | |
|---|---|---|---|---|
| HER2 | G776insYVM | LGSGAFGTVYKGIWIP | ILDEAYVMAY (A01.01) | Lung Cancer |
| | A | DGENVKIPVAIKVLRE | VMAYVMAGV (A02.01) | |
| | | NTSPKANKEILDEAYV | YVMAYVMAG (A02.01, B07.02, | |
| | | MAYVMAGVGSPYVS | B08.01) | |
| | | RLLGICLTSTVQLVTQ | YVMAYVMAGV (A02.01, | |
| | | LMPYGCLLDHVRENR | B07.02, B08.01) | |
| | | GRLGSQDLLNW | | |

¹Underlined AAs represent non-native AAs
²Bolded AAs represent native AAs of the amino acid sequence encoded by the second of the two fused genes
³Bolded and underlined AAs represent non-native AAs of the amino acid sequence encoded by the second of the two fused genes due to a frameshift.

A very common mutation to ibrutinib, a molecule targeting Bruton's Tyrosine Kinase (BTK) and used for CLL and certain lymphomas, is a Cysteine to Serine change at position 481 (C481S). This change produces a number of binding peptides which bind to a range of HLA molecules. The mutation is harbored in a region having the amino acid sequence: IFIITEYMANGSLLNYLREMRHR, the mutated Serine is underlined.

Exemplary neoantigenic peptides corresponding to the C481S mutation are presented in Table 34. The table also provides a list of HLA alleles, the encoded protein products of which can bind to the peptides. In some embodiments, the disclosure provides C481S neoepitopes for cancer therapeutics, such as, ANGSLLNY; ANGSLLNYL; ANGSLLNYLR; EYMANGSL; EYMANGSLLN; EYMANGSLLNY; GSLLNYLR; GSLLNYLREM; ITEYMANGS; ITEYMANGSL; ITEYMANGSLL; MANGSLLNYL; MANGSLLNYLR; NGSLLNYL; NGSLLNYL; SLLNYLREMR; TEYMANGSLL; TEYMANGSLLNY; YMANGSLL; and YMANGSLLN. Tables 35 and 3 provide exemplary neoantigen candidates corresponding to other cancer associated gene mutations. Table 36 provides a list of selected HLA-restricted BTK peptides for the purpose of this Application and the corresponding protein encoded by the HLA allele to which the mutant BTK peptide binds or is predicted to bind. Table 37 provides a list of selected BTK peptides and the corresponding preferred protein encoded by the HLA allele to which the peptide binds or is predicted to bind, as applicable to the context of this Application.

### Table 34 Below Lists Exemplary Neoantigenic Peptides Corresponding to the C481S Mutation

| BTK Peptides | HLA allele |
|---|---|
| ANGSLLNY | HLA-A36:01 |
| ANGSLLNYL | HLA-C15:02; HLA-008:01; HLA-006:02; |
| | HLA-A02:04; HLA-C12:02; |
| | HLA-B44:02; HLA-C17:01; HLA-B38:01 |
| ANGSLLNYLR | HLA-A74 :01, HLA-A31:01 |
| EYMANGSL | HLA-C14:02; HLA-C14:03; HLA-A24:02 |
| EYMANGSLL | HLA-A24:02; HLA-A23:01; HLA-A68:04; |
| | HLA-C14:02, HLA-C14:03, HLA-A33:03, |
| | HLA-004:01, HLA-B15:09, HLA-B38:01, |
| EYMANGSLLN | HLA-A23:01, HLA-A24:02 |
| EYMANGSLLNY | HLA-A29:02 |
| GSLLNYLR | HLA-A74:01, HLA-A31:01 |
| GSLLNYLREM | HLA-B57:01, HLA-B58:02 |
| ITEYMANGS | HLA-A01:01 |
| ITEYMANGSL | HLA-A01:01 |
| ITEYMANGSLL | HLA-A01:01 |
| MANGSLLNY | HLA-C02:02, HLA-C03:02, HLA-B53:01, |
| | HLA-C12:02, HLA-C12:03, HLA-A36:01, |
| | HLA-A26:01, HLA-A25:01, HLA-A03:01, |
| | HLA-B46:01, HLA-B15:03, HLA-A33:03, |
| | HLA-B35:03, HLA-A11:01, HLA-B15:01, |
| | HLA-B35:03, HLA-A29:02, HLA-B58:01, |
| | HLA-A30:02, HLA-B35:01 |
| MANGSLLNYL | HLA-C17:01, HLA-CO2:02, HLA-B35:01, |
| | HLA-C03:03, HLA-C08:01, HLA-B35:03, |
| | HLA-C12:02, HLA-C01:02, HLA-C03:04, |
| | HLA-C08:02 |
| MANGSLLNYLR | HLA-A33:03, HLA-A74:01 |
| NGSLLNYL | HLA-B14:02 |
| NGSLLNYLR | HLA-A68:01, HLA-A33:03, HLA-A31:01, |
| | HLA-A74:01 |
| SLLNYLREM | HLA-A02:04, HLA-A02:03, HLA-C03:02, |
| | HLA-A03:01, HLA-A32:01, HLA-A02:07, |
| | HLA-C14:03, HLA-C14:02, HLA-A31:01, |
| | HLA-A30:02, HLA-A74:01, HLA-C06:02, |
| | HLA-B15:03, HLA-B46:01, HLA-B13:02, |
| | HLA-A25:01, HLA-A29:02, HLA-C01:02, |
| | HLA-A02:01 |
| SLLNYLREMR | HLA-A74:01, HLA-A31:01 |
| TEYMANGSL | HLA-B14:02, HLA-B49:01, HLA-B44:03, |
| | HLA-B44:02, HLA-B37:01, HLA-B15:09, |
| | HLA-B41:01, HLA-B50:01, HLA-B18:01, |
| | HLA-B40:01, HLA-B40:02 |
| TEYMANGSLL | HLA-B40:02, HLA-B44:03, HLA-B49:01, |
| | HLA-B44:02, HLA-B49:01 |
| TEYMANGSLLNY | HLA-B44:03 |
| YMANGSLL | HLA-A01:01, HLA-C02:02, HLA-C04:01, |
| | HLA-C14:02, HLA-C14:03, HLA-C03:02, |
| | HLA-C17:01, HLA-C03:03, HLA-C03:04, |
| | HLA-B15:09 |
| YMANGSLLN | HLA-A01:01, HLA-A29:02 |
| YMANGSLLNY | HLA-A29:02, HLA-A36:01, HLA-B46:01, |
| | HLA-A25:01, HLA-B15:01, HLA-A26:01, |
| | HLA-A30:02, HLA-A32:01 |

TABLES 35
provide exemplary neoantigen candidates corresponding to other cancer associated gene mutations

| | | | | |
|---|---|---|---|---|
| | Exemplary | | | |
| | Protein | Mutation Sequence | Peptides (HLA allele | |
| Gene | Change | Context | example(s)) | Exemplary Diseases |

TABLE 35A

| | | POINT MUTATION ¹ | | |
|---|---|---|---|---|
| ABL1 | E255K | VADGLITTLHYPAPKR | GQYGKVYEG (A02.01) | Chronic myeloid |
| | | NKPTVYGVSPNYDKW | GQYGKVYEGV | leukemia (CML), |
| | | EMERTDITMKHKLGG | (A02.01) | Acute lymphocytic |
| | | GQYGKVYEGVWKKY | KLGGGQYGK (A03.01) | leukemia (ALL), |
| | | SLTVAVKTLKEDTME | KLGGGQYGKV | Gastrointestinal |
| | | VEEFLKEAAVMKEIK | (A02.01) | stromal tumors (GIST) |
| | | HPNLVQLLGVC | KVYEGVWKK (A02.01, | |
| | | | A03.01) | |
| | | | KVYEGVWKKY | |
| | | | (A03.01) | |
| | | | QYGKVYEGV (A24.02) | |
| | | | QYGKVYEGVW | |
| | | | (A24.02) | |
| ABL1 | E255V | VADGLITTLHYPAPKR | GQYGVVYEG (A02.01) | Chronic myeloid |
| | | NKPTVYGVSPNYDKW | GQYGVVYEGV | leukemia (CML), |
| | | EMERTDITMKHKLGG | (A02.01) | Acute lymphocytic |
| | | GQYGVVYEGVWKKY | KLGGGQYGV (A02.01) | leukemia (ALL), |
| | | SLTVAVKTLKEDTME | KLGGGQYGVV | Gastrointestinal |
| | | VEEFLKEAAVMKEIK | (A02. 01) | stromal tumors (GIST) |
| | | HPNLVQLLGVC | QYGVVYEGV (A24.02) | |
| | | | QYGVVYEGVW | |
| | | | (A24.02) | |
| | | | VVYEGVWKK (A02.01, | |
| | | | A03.01) | |
| | | | VVYEGVWKKY | |
| | | | (A03.01) | |
| ABL1 | M351T | LLGVCTREPPFYIITEF | ATQISSATEY (A01.01) | Chronic myeloid |
| | | MTYGNLLDYLRECNR | ISSATEYLEK (A03.01) | leukemia (CML), |
| | | QEVNAVVLLYMATQI | SSATEYLEK (A03.01) | Acute lymphocytic |
| | | SSATEYLEKKNFIHRD | TQISSATEYL (A02.01) | leukemia (ALL), |
| | | LAARNCLVGENHLVK | YMATQISSAT (A02.01) | Gastrointestinal |
| | | VADFGLSRLMTGDTY | | stromal tumors (GIST) |
| | | TAHAGAKF | | |
| ABL1 | T315I | SLTVAVKTLKEDTME | FYIIIEFMTY (A24.02) | Chronic myeloid |
| | | VEEFLKEAAVMKEIK | IIEFMTYGNL (A02.01) | leukemia (CML), |
| | | HPNLVQLLGVCTREPP | IIIEFMTYG (A02.01) | Acute lymphocytic |
| | | FYIIIEFMTYGNLLDYL | IIIEFMTYGN (A02.01) | leukemia (ALL), |
| | | RECNRQEVNAVVLLY | YIIIEFMTYG (A02.01) | Gastrointestinal |
| | | MATQISSAMEYLEKK | | stromal tumors (GIST) |
| | | NFIHRDLA | | |
| ABL1 | Y253H | STVADGLITTLHYPAP | GQHGEVYEGV | Chronic myeloid |
| | | KRNKPTVYGVSPNYD | (A02.01) | leukemia (CML), |
| | | KWEMERTDITMKHKL | KLGGGQHGEV | Acute lymphocytic |
| | | GGGQHGEVYEGVWK | (A02.01) | leukemia (ALL), |
| | | KYSLTVAVKTLKEDT | | Gastrointestinal |
| | | MEVEEFLKEAAVMKE | | stromal tumors (GIST) |
| | | IKHPNLVQLLG | | |
| ALK | G1269A | SSLAMLDLLHVARDI | KIADFGMAR (A03.01) | NSCLC |
| | | ACGCQYLEENHFIHR | RVAKIADFGM | |
| | | DIAARNCLLTCPGPGR | (A02.01, B07.02) | |
| | | VAKIADFGMARDIYR | | |
| | | ASYYRKGGCAMLPVK | | |
| | | WMPPEAFMEGIFTSKT | | |
| | | DTWSFGVLL | | |
| ALK | L1196M | QVAVKTLPEVCSEQD | FILMELMAGG | NSCLC |
| | | ELDFLMEALIISKFNH | (A02.01) | |
| | | QNIVRCIGVSLQSLPRF | ILMELMAGG (A02.01) | |
| | | ILMELMAGGDLKSFL | ILMELMAGGD | |
| | | RETRPRPSQPSSLAML | (A02.01) | |
| | | DLLHVARDIACGCQY | LMELMAGGDL | |
| | | LEENHFI | (A02.01) | |
| | | | LPRFILMEL (B07.02, | |
| | | | B08.01) | |
| | | | LPRFILMELM (B07.02) | |
| | | | LQSLPRFILM (A02.01, | |
| | | | B08.01) | |
| | | | SLPRFILMEL (A02.01, | |
| | | | A24.02, B07.02, B08.01) | |
| BRAF | V600E | MIKLIDIARQTAQGMD | LATEKSRWS (A02.01, | CRC, GBM, KIRP, |
| | | YLHAKSIIHRDLKSNN | B08.01) | LUAD, SKCM, |
| | | IFLHEDLTVKIGDFGL | LATEKSRWSG | THCA |
| | | ATEKSRWSGSHQFEQ | (A02.01, B08.01) | |
| | | LSGSILWMAPEVIRMQ | | |
| | | DKNPYSFQSDVYAFGI | | |
| | | VLYELM | | |
| BTK | C481S | MIKEGSMSEDEFIEEA | EYMANGSLL (A24.02) | BTK |
| | | KVMMNLSHEKLVQL | | |
| | | YGVCTKQRPIFIITEY | | |
| | | MANGSLLNYLREMRH | | |
| | | RFQTQQLLEMCKDVC | | |
| | | EAMEYLESKQFLHRD | | |
| | | LAARNCLVND | | |
| EEF1B2 | S43G | MGFGDLKSPAGLQVL | GPPPADLCHAL | BLCA, KIRP, PRAD, |
| | | NDYLADKSYIEGYVPS | (B07.02) | SKCM |
| | | QADVAVFEAVSGPPP | | |
| | | ADLCHALRWYNHIKS | | |
| | | YEKEKASLPGVKKAL | | |
| | | GKYGPADVEDTTGSG | | |
| | | AT | | |
| ERBB3 | V104M | ERCEVVMGNLEIVLT | | CRC, Stomach Cancer |
| | | GHNADLSFLQWIREV | | |
| | | TGYVLVAMNEFSTLP | | |
| | | LPNLRMVRGTQVYDG | | |
| | | KFAIFVMLNYNTNSSH | | |
| | | ALRQLRLTQLTEILSG | | |
| | | GVYIEKNDK | | |
| ESR1 | D538G | HLMAKAGLTLQQQH | GLLLEMLDA (A02.01) | Breast Cancer |
| | | QRLAQLLLILSHIRHM | LYGLLLEML (A24.02) | |
| | | SNKGMEHLYSMKCK | NVVPLYGLL (A02.01) | |
| | | NVVPLYGLLLEMLDA | PLYGLLLEM (A02.01) | |
| | | HRLHAPTSRGGASVE | PLYGLLLEML (A02.01, | |
| | | ETDQSHLATAGSTSSH | A24.02) | |
| | | SLQKYYITGEA | VPLYGLLLEM (B07.02) | |
| | | | VVPLYGLLL (A02.01, | |
| | | | A24.02) | |
| ESR1 | S463P | NQGKCVEGMVEIFDM | FLPSTLKSL (A02.01, | Breast Cancer |
| | | LLATSSRFRMMNLQG | A24.02, B08.01) | |
| | | EEFVCLKSIILLNSGVY | GVYTFLPST (A02.01) | |
| | | TFLPSTLKSLEEKDHIH | GVYTFLPSTL (A02.01, | |
| | | RVLDKITDTLIHLMAK | A24.02) | |
| | | AGLTLQQQHQRLAQL | TFLPSTLKSL (A24.02) | |
| | | LLILSH | VYTFLPSTL (A24.02) | |
| | | | YTFLPSTLK (A03.01) | |
| ESR1 | Y537C | IHLMAKAGLTLQQQH | NVVPLCDLL (A02.01) | Breast Cancer |
| | | QRLAQLLLILSHIRHM | NVVPLCDLLL (A02.01) | |
| | | SNKGMEHLYSMKCK | PLCDLLLEM (A02.01) | |
| | | NVVPLCDLLLEMLDA | PLCDLLLEML (A02.01) | |
| | | HRLHAPTSRGGASVE | VPLCDLLLEM (B07.02) | |
| | | ETDQSHLATAGSTSSH | VVPLCDLLL (A02.01, | |
| | | SLQKYYITGE | A24.02) | |
| ESR1 | Y537N | IHLMAKAGLTLQQQH | NVVPLNDLL (A02.01) | Breast Cancer |
| | | QRLAQLLLILSHIRHM | NVVPLNDLLL (A02.01) | |
| | | SNKGMEHLYSMKCK | PLNDLLLEM (A02.01) | |
| | | NVVPLNDLLLEMLDA | PLNDLLLEML (A02.01) | |
| | | HRLHAPTSRGGASVE | VPLNDLLLEM (B07.02) | |
| | | ETDQSHLATAGSTSSH | | |
| | | SLQKYYITGE | | |
| ESR1 | Y537S | IHLMAKAGLTLQQQH | NVVPLSDLL (A02.01) | Breast Cancer |
| | | QRLAQLLLILSHIRHM | NVVPLSDLLL (A02.01) | |
| | | SNKGMEHLYSMKCK | PLSDLLLEM (A02.01) | |
| | | NVVPLSDLLLEMLDA | PLSDLLLEML (A02.01) | |
| | | HRLHAPTSRGGASVE | VPLSDLLLEM (B07.02) | |
| | | ETDQSHLATAGSTSSH | VVPLSDLLL (A02.01, | |
| | | SLQKYYITGE | A24.02) | |
| FGFR3 | S249C | HRIGGIKLRHQQWSL | VLERCPHRPI (A02.01, | BLCA, HNSC, KIRP, |
| | | VMESVVPSDRGNYTC | B08.01) | LUSC |
| | | VVENKFGSIRQTYTLD | YTLDVLERC (A02.01) | |
| | | VLERCPHRPILQAGLP | | |
| | | ANQTAVLGSDVEFHC | | |
| | | KVYSDAQPHIQWLKH | | |
| | | VEVNGSKVG | | |
| FRG1B | L52S | AVKLSDSRIALKSGYG | FQNGKMALS (A02.01) | GBM, KIRP, PRAD, |
| | | KYLGINSDELVGHSD | | SKCM |
| | | AIGPREQWEPVFQNG | | |
| | | KMALSASNSCFIRCNE | | |
| | | AGDIEAKSKTAGEEE | | |
| | | MIKIRSCAEKETKKKD | | |
| | | DIPEEDKG | | |
| HER2 | V777L | GSGAFGTVYKGIWIPD | VMAGLGSPYV | BRCA |
| | (Resistance) | GENVKIPVAIKVLREN | (A02.01, A03.01) | |
| | | TSPKANKEILDEAYV | | |
| | | MAGLGSPYVSRLLGIC | | |
| | | LTSTVQLVTQLMPYG | | |
| | | CLLDHVRENRGRLGS | | |
| | | QDLLNWCM | | |
| IDH1 | R132H | RVEEFKLKQMWKSPN | KPIIIGHHA (B07.02) | BLCA, GBM, PRAD |
| | | GTIRNILGGTVFREAII | | |
| | | CKNIPRLVSGWVKPIII | | |
| | | GHHAYGDQYRATDF | | |
| | | VVPGPGKVEITYTPSD | | |
| | | GTQKVTYLVHNFEEG | | |
| | | GGVAMGM | | |
| IDH1 | R132C | RVEEFKLKQMWKSPN | KPIIIGCHA (B07.02) | BLCA, GBM, PRAD |
| | | GTIRNILGGTVFREAII | | |
| | | CKNIPRLVSGWVKPIII | | |
| | | GCHAYGDQYRATDFV | | |
| | | VPGPGKVEITYTPSDG | | |
| | | TQKVTYLVHNFEEGG | | |
| | | GVAMGM | | |
| IDH1 | R132G | RVEEFKLKQMWKSPN | KPIIIGGHA (B07.02) | BLCA, BRCA, CRC, |
| | | GTIRNILGGTVFREAII | | GBM, HNSC, LUAD, |
| | | CKNIPRLVSGWVKPIII | | PAAD, PRAD, UCEC |
| | | GGHAYGDQYRATDF | | |
| | | VVPGPGKVEITYTPSD | | |
| | | GTQKVTYLVHNFEEG | | |
| | | GGVAMGM | | |
| IDH1 | R132S | RVEEFKLKQMWKSPN | KPIIIGSHA (B07.02) | BLCA, BRCA, GBM, |
| | | GTIRNILGGTVFREAII | | HNSC, LIHC, LUAD, |
| | | CKNIPRLVSGWVKPIII | | LUSC, PAAD, |
| | | GSHAYGDQYRATDFV | | SKCM, UCEC |
| | | VPGPGKVEITYTPSDG | | |
| | | TQKVTYLVHNFEEGG | | |
| | | GVAMGM | | |
| KIT | T670I | VAVKMLKPSAHLTER | IIEYCCYGDL (A02.01) | Gastrointestinal |
| | | EALMSELKVLSYLGN | TIGGPTLVII (A02.01) | stromal tumors (GIST) |
| | | HMNIVNLLGACTIGGP | VIIEYCCYG (A02.01) | |
| | | TLVIIEYCCYGDLLNF | | |
| | | LRRKRDSFICSKQEDH | | |
| | | AEAALYKNLLHSKES | | |
| | | SCSDSTNE | | |
| KIT | V654A | VEATAYGLIKSDAAM | HMNIANLLGA | Gastrointestinal |
| | | TVAVKMLKPSAHLTE | (A02.01) | stromal tumors (GIST) |
| | | REALMSELKVLSYLG | IANLLGACTI (A02.01) | |
| | | NHMNIANLLGACTIG | MNIANLLGA (A02.01) | |
| | | GPTLVITEYCCYGDLL | YLGNHMNIA (A02.01, | |
| | | NFLRRKRDSFICSKQE | B08.01) | |
| | | DHAEAALYK | YLGNHMNIAN | |
| | | | (A02.01) | |
| MEK | C121S | ISELGAGNGGVVFKVS | VLHESNSPY (A03.01) | Melanoma |
| | | HKPSGLVMARKLIHL | VLHESNSPYI (A02.01) | |
| | | EIKPAIRNQIIRELQVL | | |
| | | HESNSPYIVGFYGAFY | | |
| | | SDGEISICMEHMDGGS | | |
| | | LDQVLKKAGRIPEQIL | | |
| | | GKVSI | | |
| MEK | P124L | LGAGNGGVVFKVSHK | LQVLHECNSL (A02.01, | Melanoma |
| | | PSGLVMARKLIHLEIK | B08.01) | |
| | | PAIRNQIIRELQVLHEC | LYIVGFYGAF (A24.02) | |
| | | NSLYIVGFYGAFYSDG | NSLYIVGFY (A01.01) | |
| | | EISICMEHMDGGSLDQ | QVLHECNSL (A02.01, | |
| | | VLKKAGRIPEQILGKV | B08.01) | |
| | | SIAVI | SLYIVGFYG (A02.01) | |
| | | | SLYIVGFYGA (A02.01) | |
| | | | VLHECNSLY (A03.01) | |
| | | | VLHECNSLYI (A02.01, | |
| | | | A03.01) | |
| MYC | E39D | MPLNVSFTNRNYDLD | FYQQQQQSDL | Lymphoid Cancer; |
| | | YDSVQPYFYCDEEEN | (A24.02) | Burkitt Lymphoma |
| | | FYQQQQQSDLQPPAPS | QQQSDLQPPA | |
| | | EDIWKKFELLPTPPLSP | (A02. 01) | |
| | | SRRSGLCSPSYVAVTP | QQSDLQPPA (A02.01) | |
| | | FSLRGDNDGG | YQQQQQSDL (A02.01, | |
| | | | B08.01) | |
| MYC | P57S | FTNRNYDLDYDSVQP | FELLSTPPL (A02.01, | Lymphoid Cancer |
| | | YFYCDEEENFYQQQQ | B08.01) | |
| | | QSELQPPAPSEDIWKK | LLSTPPLSPS (A02.01) | |
| | | FELLSTPPLSPSRRSGL | | |
| | | CSPSYVAVTPFSLRGD | | |
| | | NDGGGGSFSTADQLE | | |
| | | MVTELLG | | |
| MYC | T58I | TNRNYDLDYDSVQPY | FELLPIPPL (A02.01) | Neuroblastoma |
| | | FYCDEEENFYQQQQQ | IWKKFELLPI (A24.02) | |
| | | SELQPPAPSEDIWKKF | LLPIPPLSPS (A02.01, | |
| | | ELLPIPPLSPSRRSGLC | B07.02) | |
| | | SPSYVAVTPFSLRGDN | LPIPPLSPS (B07.02) | |
| | | DGGGGSFSTADQLEM | | |
| | | VTELLGG | | |
| PDGFRa | T674I | VAVKMLKPTARSSEK | IIEYCFYGDL (A02.01) | Chronic Eosinophilic |
| | | QALMSELKIMTHLGP | IIIEYCFYG (A02.01) | Leukemia |
| | | HLNIVNLLGACTKSGP | IYIIIEYCF (A24.02) | |
| | | IYIIIEYCFYGDLVNYL | IYIIIEYCFY (A24.02) | |
| | | HKNRDSFLSHHPEKPK | YIIIEYCFYG (A02.01) | |
| | | KELDIFGLNPADESTR | | |
| | | SYVILS | | |
| PIK3CA | E542K | IEEHANWSVSREAGFS | KITEQEKDFL (A02.01) | BLCA, BRCA, CESC, |
| | | YSHAGLSNRLARDNE | | CRC, GBM, HNSC, |
| | | LRENDKEQLKAISTRD | | KIRC, KIRP, LIHC, |
| | | PLSKITEQEKDFLWSH | | LUAD, LUSC, PRAD, |
| | | RHYCVTIPEILPKLLLS | | UCEC |
| | | VKWNSRDEVAQMYC | | |
| | | LVKDWPP | | |
| PIK3CA | E545K | HANWSVSREAGFSYS | STRDPLSEITK (A03.01) | BLCA, BRCA, CESC, |
| | | HAGLSNRLARDNELR | DPLSEITK (A03.01) | CRC, GBM, HNSC, |
| | | ENDKEQLKAISTRDPL | | KIRC, KIRP, LIHC, |
| | | SEITKQEKDFLWSHRH | | LUAD, LUSC, PRAD, |
| | | YCVTIPEILPKLLLSVK | | SKCM, UCEC |
| | | WNSRDEVAQMYCLV | | |
| | | KDWPPIKP | | |
| PIK3CA | H1047R | LFINLFSMMLGSGMPE | | BRCA, CESC, CRC, |
| | | LQSFDDIAYIRKTLAL | | GBM, HNSC, LIHC, |
| | | DKTEQEALEYFMKQM | | LUAD, LUSC, PRAD, |
| | | NDARHGGWTTKMDW | | UCEC |
| | | IFHTIKQHALN | | |
| POLE | P286R | QRGGVITDEEETSKKI | LPLKFRDAET (B07.02) | Colorectal |
| | | ADQLDNIVDMREYDV | | adenocarcinoma; |
| | | PYHIRLSIDIETTKLPL | | Uterine/Endometrium |
| | | KFRDAETDQIMMISY | | Adenocarcinoma; |
| | | MIDGQGYLITNREIVS | | Colorectal |
| | | EDIEDFEFTPKPEYEGP | | adenocarcinoma, |
| | | FCVFN | | MSI+; |
| | | | | Uterine/Endometrium |
| | | | | Adenocarcinoma, |
| | | | | MSI+; Endometrioid |
| | | | | carcinoma; |
| | | | | Endometrium Serous |
| | | | | carcinoma; |
| | | | | Endometrium |
| | | | | Carcinosarcoma- |
| | | | | malignant mesodermal |
| | | | | mixed tumor; Glioma; |
| | | | | Astrocytoma; GBM |
| PTEN | R130Q | KFNCRVAQYPFEDHN | QTGVMICAYL | BRCA, CESC, CRC, |
| | | PPQLELIKPFCEDLDQ | (A02.01) | GBM, KIRC, LUSC, |
| | | WLSEDDNHVAAIHCK | | UCEC |
| | | AGKGQTGVMICAYLL | | |
| | | HRGKFLKAQEALDFY | | |
| | | GEVRTRDKKGVTIPSQ | | |
| | | RRYVYYYSY | | |
| RAC1 | P29S | MQAIKCVVVGDGAV | AFSGEYIPTV (A02.01, | Melanoma |
| | | GKTCLLISYTTNAFSG | A24.02) | |
| | | EYIPTVFDNYSANVM | | |
| | | VDGKPVNLGLWDTA | | |
| | | GQEDYDRLRPLSYPQ | | |
| | | TVGET | | |
| TP53 | G245S | IRVEGNLRVEYLDDR | SMNRRPILT (A02.01, | BLCA, BRCA, CRC, |
| | | NTFRHSVVVPYEPPEV | B08.01) | GBM, HNSC, LUSC, |
| | | GSDCTTIHYNYMCNS | YMCNSSCMGS | PAAD, PRAD |
| | | SCMGSMNRRPILTIITL | (A02.01) | |
| | | EDSSGNLLGRNSFEVR | | |
| | | VCACPGRDRRTEEEN | | |
| | | LRKKGEP | | |
| TP53 | R175H | TYSPALNKMFCQLAK | | BLCA, BRCA, CRC, |
| | | TCPVQLWVDSTPPPGT | | GBM, HNSC, LUAD, |
| | | RVRAMAIYKQSQHMT | | PAAD, PRAD, UCEC |
| | | EVVRHCPHHERCSDS | | |
| | | DGLAPPQHLIRVEGNL | | |
| | | RVEYLDDRNTFRHSV | | |
| | | VVPYEPPEV | | |
| TP53 | R248Q | EGNLRVEYLDDRNTF | GMNQRPILT (A02.01) | BLCA, BRCA, CRC, |
| | | RHSVVVPYEPPEVGSD | | GBM, HNSC, KIRC, |
| | | CTTIHYNYMCNSSCM | | LIHC, LUSC, PAAD, |
| | | GGMNQRPILTIITLEDS | | PRAD, UCEC |
| | | SGNLLGRNSFEVRVC | | |
| | | ACPGRDRRTEEENLR | | |
| | | KKGEPHHE | | |
| TP53 | R248W | EGNLRVEYLDDRNTF | GMNWRPILT (A02.01) | BLCA, BRCA, CRC, |
| | | RHSVVVPYEPPEVGSD | | GBM, HNSC, LIHC, |
| | | CTTIHYNYMCNSSCM | | LUSC, PAAD, |
| | | GGMNWRPILTIITLED | | SKCM, UCEC |
| | | SSGNLLGRNSFEVRVC | | |
| | | ACPGRDRRTEEENLR | | |
| | | KKGEPHHE | | |
| TP53 | R273C | PEVGSDCTTIHYNYM | LLGRNSFEVC (A02.01) | BLCA, BRCA, CRC, |
| | | CNSSCMGGMNRRPIL | | GBM, HNSC, LUSC, |
| | | TIITLEDSSGNLLGRNS | | PAAD, UCEC |
| | | FEVCVCACPGRDRRT | | |
| | | EEENLRKKGEPHHELP | | |
| | | PGSTKRALPNNTSSSP | | |
| | | QPKKKPL | | |

TABLE 35B

| | | MSI-ASSOCIATED | | |
|---|---|---|---|---|
| | | FRAMESHIFTS ¹ | | |
| ACVR2A | D96fs; +1 | GVEPCYGDKDKRRHC | | MSI+ CRC, MSI+ |
| | | FATWKNISGSIEIVKQ | | Uterine/Endometrium |
| | | GCWLDDINCYDRTDC | | Cancer, MSI+ |
| | | VEKKRQP* | | Stomach Cancer, |
| | | | | Lynch syndrome |
| ACVR2A | D96fs; −1 | GVEPCYGDKDKRRHC | ALKYIFVAV (A02.01, | MSI+ CRC, MSI+ |
| | | FATWKNISGSIEIVKQ | B08.01) | Uterine/Endometrium |
| | | GCWLDDINCYDRTDC | ALKYIFVAVR (A03.01) | Cancer, MSI+ |
| | | VEKKTALKYIFVAVR | AVRAICVMK (A03.01) | Stomach Cancer, |
| | | AICVMKSFLIFRRWKS | AVRAICVMKS | Lynch syndrome |
| | | HSPLQIQLHLSHPITTS | (A03.01) | |
| | | CSIPWCHLC* | CVEKKTALK (A03.01) | |
| | | | CVEKKTALKY | |
| | | | (A01.01) | |
| | | | CVMKSFLIF (A24.02, | |
| | | | B08.01) | |
| | | | CVMKSFLIFR (A03.01) | |
| | | | FLIFRRWKS (A02.01, | |
| | | | B08.01) | |
| | | | FRRWKSHSPL (B08.01) | |
| | | | FVAVRAICV (A02.01, | |
| | | | B08.01) | |
| | | | FVAVRAICVM | |
| | | | (B08.01) | |
| | | | IQLHLSHPI (A02.01) | |
| | | | KSFLIFRRWK (A03.01) | |
| | | | KTALKYIFV (A02.01) | |
| | | | KYIFVAVRAI (A24.02) | |
| | | | RWKSHSPLQI (A24.02) | |
| | | | TALKYIFVAV (A02.01, | |
| | | | B08.01) | |
| | | | VAVRAICVMK | |
| | | | (A03.01) | |
| | | | VMKSFLIFR (A03.01) | |
| | | | VMKSFLIFRR (A03.01) | |
| | | | YIFVAVRAI (A02.01) | |
| C15ORF40 | L132fs; +1 | TAEAVNVAIAAPPSEG | ALFFFFFET (A02.01) | MSI+ CRC, MSI+ |
| | | EANAELCRYLSKVLE | ALFFFFFETK (A03.01) | Uterine/Endometrium |
| | | LRKSDVVLDKVGLAL | AQAGVQWRSL | Cancer, MSI+ |
| | | FFFFFETKSCSVAQAG | (A02.01) | Stomach Cancer, |
| | | VQWRSLGSLQPPPPGF | CLANFCIFNR (A03.01) | Lynch syndrome |
| | | KLFSCLSFLSSWDYRR | CLSFLSSWDY (A01.01, | |
| | | MPPCLANFCIFNRDGV | A03.01) | |
| | | SPCWSGWS* | FFETKSCSV (B08.01) | |
| | | | FFFETKSCSV (A02.01) | |
| | | | FKLFSCLSFL (A02.01) | |
| | | | FLSSWDYRRM | |
| | | | (A02.01) | |
| | | | GFKLFSCLSF (A24.02) | |
| | | | KLFSCLSFL (A02.01, | |
| | | | A03.01) | |
| | | | KLFSCLSFLS (A02.01, | |
| | | | A03.01) | |
| | | | LALFFFFFET (A02.01) | |
| | | | LFFFFFETK (A03.01) | |
| | | | LSFLSSWDY (A01.01) | |
| | | | LSFLSSWDYR (A03.01) | |
| | | | RMPPCLANF (A24.02) | |
| | | | RRMPPCLANF | |
| | | | (A24.02) | |
| | | | SLQPPPPGFK (A03.01) | |
| | | | VQWRSLGSL (A02.01) | |
| CNOT1 | L1544fs; +1 | LSVIIFFFVYIWHWAL | FFFSVIFST (A02.01) | MSI+ CRC, MSI+ |
| | | PLILNNHHICLMSSIIL | MSVCFFFFSV (A02.01) | Uterine/Endometrium |
| | | DCNSVRQSIMSVCFFF | SVCFFFFSV (A02.01, | Cancer, MSI+ |
| | | FSVIFSTRCLTDSRYPN | B08.01) | Stomach Cancer, |
| | | ICWFK* | SVCFFFFSVI (A02.01) | Lynch syndrome |
| CNOT1 | L1544fs; −1 | LSVIIFFFVYIWHWAL | FFCYILNTMF (A24.02) | MSI+ CRC, MSI+ |
| | | PLILNNHHICLMSSIIL | MSVCFFFFCY (A01.01) | Uterine/Endometrium |
| | | DCNSVRQSIMSVCFFF | SVCFFFFCYI (A02.01) | Cancer, MSI+ |
| | | FCYILNTMFDR* | | Stomach Cancer, |
| | | | | Lynch syndrome |
| EIF2B3 | A151fs; −1 | VLVLSCDLITDVALHE | KQWSSVTSL (A02.01) | MSI+ CRC, MSI+ |
| | | VVDLFRAYDASLAML | VLWMPTSTV (A02.01) | Uterine/Endometrium |
| | | MRKGQDSIEPVPGQK | | Cancer, MSI+ |
| | | GKKKQWSSVTSLEWT | | Stomach Cancer, |
| | | AQERGCSSWLMKQT | | Lynch syndrome |
| | | WMKSWSLRDPSYRSI | | |
| | | LEYVSTRVLWMPTST | | |
| | | V* | | |
| EPHB2 | K1020fs; −1 | SIQVMRAQMNQIQSV | ILIRKAMTV | MSI+ CRC, MSI+ |
| | | EGQPLARRPRATGRT | (A02.01) | Uterine/Endometrium |
| | | KRCQPRDVTKKTCNS | | Cancer, MSI+ |
| | | NDGKKREWEKRKQIL | | Stomach Cancer, |
| | | GGGGKYKEYFLKRILI | | Lynch syndrome |
| | | RKAMTVLAGDKKGL | | |
| | | GRFMRCVQSETKAVS | | |
| | | LQLPLGR* | | |
| ESRP1 | N512fs; +1 | LDFLGEFATDIRTHGV | | MSI+ CRC, MSI+ |
| | | HMVLNHQGRPSGDAF | | Uterine/Endometrium |
| | | IQMKSADRAFMAAQK | | Cancer, MSI+ |
| | | CHKKKHEGQIC* | | Stomach Cancer, |
| | | | | Lynch syndrome |
| ESRP1 | N512fs; −1 | LDFLGEFATDIRTHGV | | MSI+ CRC, MSI+ |
| | | HMVLNHQGRPSGDAF | | Uterine/Endometrium |
| | | IQMKSADRAFMAAQK | | Cancer, MSI+ |
| | | CHKKT* | | Stomach Cancer, |
| | | | | Lynch syndrome |
| FAM111 | A273fs; −1 | GALCKDGRFRSDIGEF | RMKVPLMK (A03.01) | MSI+ CRC, MSI+ |
| B | | EWKLKEGHKKIYGKQ | | Uterine/Endometrium |
| | | SMVDEVSGKVLEMDI | | Cancer, MSI+ |
| | | SKKKHYNRKISIKKLN | | Stomach Cancer, |
| | | RMKVPLMKLITRV* | | Lynch syndrome |
| GBP3 | T585fs; −1 | RERAQLLEEQEKTLTS | TLKKKPRDI | MSI+ CRC, MSI+ |
| | | KLQEQARVLKERCQG | (B08.01) | Uterine/Endometrium |
| | | ESTQLQNEIQKLQKTL | | Cancer, MSI+ |
| | | KKKPRDICRIS* | | Stomach Cancer, |
| | | | | Lynch syndrome |
| JAK1 | P861fs; +1 | VNTLKEGKRLPCPPNC | LIEGFEALLK (A03.01) | MSI+ CRC, MSI+ |
| | | PDEVYQLMRKCWEFQ | | Uterine/Endometrium |
| | | PSNRTSFQNLIEGFEAL | | Cancer, MSI+ |
| | | LKTSN* | | Stomach Cancer, |
| | | | | Lynch syndrome |
| JAK1 | K860fs; −1 | CRPVTPSCKELADLM | QQLKWTPHI (A02.01) | MSI+ CRC MSI+ |
| | | TRCMNYDPNQRPFFR | QLKWTPHILK (A03.01) | Uterine/Endometnum |
| | | AIMRDINKLEEQNPDI | IVSEKNQQLK (A03.01) | Cancer, MSI+ |
| | | VSEKNQQLKWTPHIL | QLKWTPHILK (A03.01) | Stomach Cancer, |
| | | KSAS* | QQLKWTPHI (A24.02) | Lynch syndrome |
| | | | NQQLKWTPHIL | |
| | | | (B08.01) | |
| | | | NQQLKWTPHI (B08.01) | |
| | | | QLKWTPHIL (B08.01) | |
| LMAN1 | E305fs; +1 | DDHDVLSFLTFQLTEP | GPPRPPRAAC (B07.02) | MSI+ CRC, MSI+ |
| | | GKEPPTPDKEISEKEK | PPRPPRAAC (B07.02) | Uterine/Endometrium |
| | | EKYQEEFEHFQQELD | | Cancer, MSI+ |
| | | KKKRGIPEGPPRPPRA | | Stomach Cancer, |
| | | ACGGNI* | | Lynch syndrome |
| LMAN1 | E305fs; −1 | DDHDVLSFLTFQLTEP | SLRRKYLRV (B08.01) | MSI+ CRC, MSI+ |
| | | GKEPPTPDKEISEKEK | | Uterine/Endometrium |
| | | EKYQEEFEHFQQELD | | Cancer, MSI+ |
| | | KKKRNSRRATPTSKG | | Stomach Cancer, |
| | | SLRRKYLRV* | | Lynch syndrome |
| MSH3 | N385fs; +1 | TKSTLIGEDVNPLIKL | SAACHRRGCV | MSI+ CRC, MSI+ |
| | | DDAVNVDEIMTDTST | (B08.01) | Uterine/Endometrium |
| | | SYLLCISENKENVRDK | | Cancer, MSI+ |
| | | KKGQHFYWHCGSAA | | Stomach Cancer, |
| | | CHRRGCV* | | Lynch syndrome |
| MSH3 | K383fs; −1 | LYTKSTLIGEDVNPLI | ALWECSLPQA | MSI+ CRC, MSI+ |
| | | KLDDAVNVDEIMTDT | (A02.01) | Uterine/Endometrium |
| | | STSYLLCISENKENVR | CLIVSRTLL (B08.01) | Cancer, MSI+ |
| | | DKKRATFLLALWECS | CLIVSRTLLL (A02.01, | Stomach Cancer, |
| | | LPQARLCLIVSRTLLL | B08.01) | Lynch syndrome |
| | | VQS* | FLLALWECS (A02.01) | |
| | | | FLLALWECSL (A02.01, | |
| | | | B08.01) | |
| | | | IVSRTLLLV (A02.01) | |
| | | | LIVSRTLLL (A02.01, | |
| | | | B08.01) | |
| | | | LIVSRTLLLV (A02.01) | |
| | | | LLALWECSL (A02.01, | |
| | | | B08.01) | |
| | | | LPQARLCLI (B08.01, | |
| | | | B07.02) | |
| | | | LPQARLCLIV (B08.01) | |
| | | | NVRDKKRATF | |
| | | | (B08.01) | |
| | | | SLPQARLCLI (A02.01, | |
| | | | B08.01) | |
| NDUFC2 | A70fs; +1 | LPPPKLTDPRLLYIGFL | FFCWILSCK (A03.01) | MSI+ CRC, MSI+ |
| | | GYCSGLIDNLIRRRPIA | FFFCWILSCK (A03.01) | Uterine/Endometrium |
| | | TAGLHRQLLYITAFFF | ITAFFFCWI (A02.01) | Cancer, MSI+ |
| | | CWILSCKT* | LYITAFFFCW (A24.02) | Stomach Cancer, |
| | | | YITAFFFCWI (A02.01) | Lynch syndrome |
| NDUFC2 | F69fs; −1 | SLPPPKLTDPRLLYIGF | ITAFFLLDI (A02.01) | MSI+ CRC, MSI+ |
| | | LGYCSGLIDNLIRRRPI | LLYITAFFL (A02.01, | Uterine/Endometrium |
| | | ATAGLHRQLLYITAFF | B08.01) | Cancer, MSI+ |
| | | LLDIIL* | LLYITAFFLL (A02.01, | Stomach Cancer, |
| | | | A24. 02) | Lynch syndrome |
| | | | LYITAFFLL (A24.02) | |
| | | | LYITAFFLLD (A24.02) | |
| | | | YITAFFLLDI (A02.01) | |
| RBM27 | Q817; +1 | NQSGGAGEDCQIFSTP | GSNEVTTRY (A01.01) | MSI+ CRC, MSI+ |
| | | GHPKMIYSSSNLKTPS | MPKDVNIQV (B07.02) | Uterine/Endometrium |
| | | KLCSGSKSHDVQEVL | TGSNEVTTRY (A01.01) | Cancer, MSI+ |
| | | KKKTGSNEVTTRYEE | | Stomach Cancer, |
| | | KKTGSVRKANRMPKD | | Lynch syndrome |
| | | VNIQVRKKQKHETRR | | |
| | | KSKYNEDFERAWRED | | |
| | | LTIKR* | | |
| RPL22 | K16fs; +1 | MAPVKKLVVKGGKK | | MSI+ CRC, MSI+ |
| | | KEASSEVHS* | | Uterine/Endometrium |
| | | | | Cancer, MSI+ |
| | | | | Stomach Cancer, |
| | | | | Lynch syndrome |
| RPL22 | K15fs; −1 | MAPVKKLVVKGGKK | | MSI+ CRC, MSI+ |
| | | RSKF* | | Uterine/Endometrium |
| | | | | Cancer, MSI+ |
| | | | | Stomach Cancer, |
| | | | | Lynch syndrome |
| SEC31A | I462fs; +1 | MPSHQGAEQQQQQH | | MSI+ CRC, MSI+ |
| | | HVFISQVVTEKEFLSR | | Uterine/Endometrium |
| | | SDQLQQAVQSQGFIN | | Cancer, MSI+ |
| | | YCQKKN* | | Stomach Cancer, |
| | | | | Lynch syndrome |
| SEC31A | I462fs; −1 | MPSHQGAEQQQQQH | KKLMLLRLNL | MSI+ CRC, MSI+ |
| | | HVFISQVVTEKEFLSR | (A02.01) | Uterine/Endometrium |
| | | SDQLQQAVQSQGFIN | KLMLLRLNL (A02.01, | Cancer, MSI+ |
| | | YCQKKLMLLRLNLRK | A03.01, B07.02, B08.01) | Stomach Cancer, |
| | | MCGPF* | KLMLLRLNLR | Lynch syndrome |
| | | | (A03.01) | |
| | | | LLRLNLRKM (B08.01) | |
| | | | LMLLRLNL (B08.01) | |
| | | | LMLLRLNLRK | |
| | | | (A03.01) | |
| | | | LNLRKMCGPF | |
| | | | (B08.01) | |
| | | | MLLRLNLRK (A03.01) | |
| | | | MLLRLNLRKM | |
| | | | (A02.01, A03.01, | |
| | | | B08.01) | |
| | | | NLRKMCGPF (B08.01) | |
| | | | NYCQKKLMLL | |
| | | | (A24.02) | |
| | | | YCQKKLMLL (B08.01) | |
| SEC63 | K530fs; +1 | AEVFEKEQSICAAEEQ | FKKKTYTCAI (B08.01) | MSI+ CRC, MSI+ |
| | | PAEDGQGETNKNRTK | ITTVKATETK (A03.01) | Uterine/Endometrium |
| | | GGWQQKSKGPKKTA | KSKKKETFK (A03.01) | Cancer, MSI+ |
| | | KSKKKETFKKKTYTC | KSKKKETFKK | Stomach Cancer, |
| | | AITTVKATETKAGKW | (A03.01) | Lynch syndrome |
| | | SRWE* | KTYTCAITTV (A02.01, | |
| | | | A24.02) | |
| | | | TFKKKTYTC (B08.01) | |
| | | | TYTCAITTV (A24.02) | |
| | | | TYTCAITTVK (A03.01) | |
| | | | YTCAITTVK (A03.01) | |
| SEC63 | K529fs; −1 | MAEVFEKEQSICAAEE | TAKSKKRNL (B08.01) | MSI+ CRC, MSI+ |
| | | QPAEDGQGETNKNRT | | Uterine/Endometrium |
| | | KGGWQQKSKGPKKT | | Cancer, MSI+ |
| | | AKSKKRNL* | | Stomach Cancer, |
| | | | | Lynch syndrome |
| SLC35F5 | C248fs; −1 | NIMEIRQLPSSHALEA | FALCGFWQI (A02.01) | MSI+ CRC, MSI+ |
| | | KLSRMSYPVKEQESIL | | Uterine/Endometrium |
| | | KTVGKLTATQVAKISF | | Cancer, MSI+ |
| | | FFALCGFWQICHIKKH | | Stomach Cancer, |
| | | FQTHKLL* | | Lynch syndrome |
| SMAP1 | K172fs; +1 | YEKKKYYDKNAIAIT | | MSI+ CRC, MSI+ |
| | | NISSSDAPLQPLVSSPS | | Uterine/Endometrium |
| | | LQAAVDKNKLEKEKE | | Cancer, MSI+ |
| | | KKKGREKERKGARKA | | Stomach Cancer, |
| | | GKTTYS* | | Lynch syndrome |
| SMAP1 | K171fs; −1 | KYEKKKYYDKNAIAI | LKKLRSPL (B08.01) | MSI+ CRC, MSI+ |
| | | TNISSSDAPLQPLVSSP | SLKKVPAL (B08.01) | Uterine/Endometrium |
| | | SLQAAVDKNKLEKEK | RKISNWSLKK (A03.01) | Cancer, MSI+ |
| | | EKKRKRKREKRSQKS | VPALKKLRSPL | Stomach Cancer, |
| | | RQNHLQLKSCRRKISN | (B07.02) | Lynch syndrome |
| | | WSLKKVPALKKLRSP | | |
| | | LWIF* | | |
| TFAM | E148fs; +1 | IYQDAYRAEWQVYKE | KRVNTAWKTK | MSI+ CRC, MSI+ |
| | | EISRFKEQLTPSQIMSL | (A03.01) | Uterine/Endometrium |
| | | EKEIMDKHLKRKAMT | MTKKKRVNTA | Cancer, MSI+ |
| | | KKKRVNTAWKTKKT | (B08.01) | Stomach Cancer, |
| | | SFSL* | RVNTAWKTK (A03.01) | Lynch syndrome |
| | | | RVNTAWKTKK | |
| | | | (A03.01) | |
| | | | TKKKRVNTA (B08.01) | |
| | | | WKTKKTSFSL (B08.01) | |
| TFAM | E148fs; −1 | IYQDAYRAEWQVYKE | | MSI+ CRC, MSI+ |
| | | EISRFKEQLTPSQIMSL | | Uterine/Endometrium |
| | | EKEIMDKHLKRKAMT | | Cancer, MSI+ |
| | | KKKS* | | Stomach Cancer, |
| | | | | Lynch syndrome |
| TGFBR2 | P129fs; +1 | KPQEVCVAVWRKND | | MSI+ CRC, MSI+ |
| | | ENITLETVCHDPKLPY | | Uterine/Endometrium |
| | | HDFILEDAASPKCIMK | | Cancer, MSI+ |
| | | EKKKAW* | | Stomach Cancer, |
| | | | | Lynch syndrome |
| TGFBR2 | K128fs: −1 | EKPQEVCVAVWRKN | ALMSAMTTS (A02.01) | MSI+ CRC, MSI+ |
| | | DENITLETVCHDPKLP | AMTTSSSQK (A03.01, | Uterine/Endometrium |
| | | YHDFILEDAASPKCIM | A11.01) | Cancer, MSI+ |
| | | KEKKSLVRLSSCVPVA | AMTTSSSQKN | Stomach Cancer, |
| | | LMSAMTTSSSQKNITP | (A03.01) | Lynch syndrome |
| | | AILTCC* | CIMKEKKSL (B08.01) | |
| | | | CIMKEKKSLV (B08.01) | |
| | | | IMKEKKSL (B08.01) | |
| | | | IMKEKKSLV (B08.01) | |
| | | | KSLVRLSSCV (A02.01) | |
| | | | LVRLSSCVPV (A02.01) | |
| | | | RLSSCVPVA (A02.01, | |
| | | | A03.01) | |
| | | | RLSSCVPVAL (A02.01) | |
| | | | SAMTTSSSQK (A03.01, | |
| | | | A11.01) | |
| | | | SLVRLSSCV (A02.01) | |
| | | | VPVALMSAM (B07.02) | |
| | | | VRLSSCVPVA (A02.01) | |
| THAP5 | K99fs; −1 | VPSKYQFLCSDHFTPD | KMRKKYAQK (A03.01) | MSI+ CRC, MSI+ |
| | | SLDIRWGIRYLKQTAV | | Uterine/Endometrium |
| | | PTIFSLPEDNQGKDPS | | Cancer, MSI+ |
| | | KKNPRRKTWKMRKK | | Stomach Cancer, |
| | | YAQKPSQKNHLY* | | Lynch syndrome |
| TTK | R854fs; −1 | GTTEEMKYVLGQLVG | FVMSDTTYK (A03.01) | MSI+ CRC, MSI+ |
| | | LNSPNSILKAAKTLYE | FVMSDTTYKI (A02.01) | Uterine/Endometrium |
| | | HYSGGESHNSSSSKTF | KTFEKKGEK (A03.01) | Cancer, MSI+ |
| | | EKKGEKNDLQLFVMS | LFVMSDTTYK | Stomach Cancer, |
| | | DTTYKIYWTVILLNPC | (A03.01) | Lynch syndrome |
| | | GNLHLKTTSL* | MSDTTYKIY (A01.01) | |
| | | | VMSDTTYKI (A02.01) | |
| | | | VMSDTTYKIY (A01.01) | |
| XPOT | F126fs; −1 | QQLIRETLISWLQAQM | YLTKWPKFFL (A02.01) | MSI+ CRC, MSI+ |
| | | LNPQPEKTFIRNKAAQ | | Uterine/Endometrium |
| | | VFALLFVTEYLTKWP | | Cancer, MSI+ |
| | | KFFLTFSQ* | | Stomach Cancer, |
| | | | | Lynch syndrome |

TABLE 35C

| | | FRAMESHIFT ¹ | | |
|---|---|---|---|---|
| APC | V1352fs | AKFQQCHSTLEPNPA | FLQERNLPP (A02.01) | CRC, LUAD, UCEC, |
| | F1354fs | DCRVLVYLQNQPGTK | FRRPHSCLA (B08.01) | STAD |
| | Q1378fs | LLNFLQERNLPPKVVL | LIVLRVVRL (B08.01) | |
| | S1398fs | RHPKVHLNTMFRRPH | LLSVHLIVL (A02.01, | |
| | | SCLADVLLSVHLIVLR | B08.01) | |
| | | VVRLPAPFRVNHAVE | | |
| | | W* | | |
| APC | S1421fs | APVIFQIALDKPCHQA | EVKHLHEILL (B08.01) | CRC, LUAD, UCEC, |
| | R1435fs | EVKHLHEILLKQLKPS | HLHEILLKQLK | STAD |
| | T1438fs | EKYLKIKHLLLKRERV | (A03.01) | |
| | P1442fs | DLSKLQ* | HLLLKRERV (B08.01) | |
| | P1443fs | | KIKHLLLKR (A03.01) | |
| | V1452fs | | KPSEKYLKI (B07.02) | |
| | P1453fs | | KYLKIKHLL (A24.02) | |
| | K1462fs | | KYLKIKHLLL (A24.02) | |
| | E1464fs | | LLKQLKPSEK (A03.01) | |
| | | | LLKRERVDL (B08.01) | |
| | | | LLLKRERVDL (B08.01) | |
| | | | QLKPSEKYLK (A03.01) | |
| | | | YLKIKHLLL (A02.01, | |
| | | | B08.01) | |
| | | | YLKIKHLLLK (A03.01) | |
| APC | T1487fs | MLQFRGSRFFQMLILY | ILPRKVLQM (B08.01) | CRC, LUAD, UCEC, |
| | H1490fs | YILPRKVLQMDFLVHP | KVLQMDFLV (A02.01, | STAD |
| | L1488fs | A* | A24.02) | |
| | | | LPRKVLQMDF | |
| | | | (B07.02, B08.01) | |
| | | | LQMDFLVHPA | |
| | | | (A02.01) | |
| | | | QMDFLVHPA (A02.01) | |
| | | | YILPRKVLQM (A02.01, | |
| | | | B08.01) | |
| ARID1A | Q1306fs | ALGPHSRISCLPTQTR | APSPASRLQC (B07.02) | STAD, UCEC, BLCA, |
| | S1316fs | GCILLAATPRSSSSSSS | HPLAPMPSKT (B07.02) | BRCA, LUSC, CESC, |
| | Y1324fs | NDMIPMAISSPPKAPL | ILPLPQLLL (A02.01) | KIRC, UCS |
| | T1348fs | LAAPSPASRLQCINSN | LLLSADQQA (A02.01) | |
| | G1351fs | SRITSGQWMAHMALL | LPTQTRGCI (B07.02) | |
| | G1378fs | PSGTKGRCTACHTAL | LPTQTRGCIL (B07.02) | |
| | P1467fs | GRGSLSSSSCPQPSPSL | RISCLPTQTR (A03.01) | |
| | | PASNKLPSLPLSKMYT | SLAETVSLH (A03.01) | |
| | | TSMAMPILPLPQLLLS | TPRSSSSSS (B07.02) | |
| | | ADQQAAPRTNFHSSL | TPRSSSSSSS (B07.02) | |
| | | AETVSLHPLAPMPSKT | | |
| | | CHHK* | | |
| ARID1A | S674fs | AHQGFPAAKESRVIQL | ALPPVLLSL (A02.01) | STAD, UCEC, BLCA, |
| | P725fs | SLLSLLIPPLTCLASEA | ALPPVLLSLA (A02.01) | BRCA, LUSC, CESC, |
| | R727fs | LPRPLLALPPVLLSLA | ALPRPLLAL (A02.01) | KIRC, UCS |
| | I736fs | QDHSRLLQCQATRCH | ASRTASCIL (B07.02) | |
| | | LGHPVASRTASCILP* | EALPRPLLAL (B08.01) | |
| | | | HLGHPVASR (A03.01) | |
| | | | HPVASRTAS (B07.02) | |
| | | | HPVASRTASC (B07.02) | |
| | | | IIQLSLLSLL (A02.01) | |
| | | | IQLSLLSLL (A02.01) | |
| | | | IQLSLLSLLI (A02.01, | |
| | | | A24.02) | |
| | | | LLALPPVLL (A02.01) | |
| | | | LLIPPLTCL (A02.01) | |
| | | | LLIPPLTCLA (A02.01) | |
| | | | LLSLLIPPL (A02.01) | |
| | | | LLSLLIPPLT (A02.01) | |
| | | | LPRPLLALPP (B07.02) | |
| | | | QLSLLSLLI (A02.01) | |
| | | | RLLQCQATR (A03.01) | |
| | | | RPLLALPPV (B07.02) | |
| | | | RPLLALPPVL (B07.02) | |
| | | | SLAQDHSRL (A02.01) | |
| | | | SLAQDHSRLL (A02.01) | |
| | | | SLLIPPLTCL (A02.01) | |
| | | | SLLSLLIPP (A02.01) | |
| | | | SLLSLLIPPL (A02.01, | |
| | | | B08.01) | |
| ARID1A | G414fs | PILAATGTSVRTAART | AAATSAASTL (B07.02) | STAD, UCEC, BLCA, |
| | Q473fs | WVPRAAIRVPDPAAV | AAIPASTSAV (B07.02) | BRCA, LUSC, CESC, |
| | H477fs | PDDHAGPGAECHGRP | AIPASTSAV (A02.01) | KIRC, UCS |
| | S499fs | LLYTADSSLWTTRPQ | ALPAGCVSSA (A02.01) | |
| | P504fs | RVWSTGPDSILQPAKS | APLLTATGSV (B07.02) | |
| | Q548fs | SPSAAAATLLPATTVP | APVLSASIL (B07.02) | |
| | P549fs | DPSCPTFVSAAATVST | ATLLPATTV (A02.01) | |
| | | TTAPVLSASILPAAIPA | ATVSTTTAPV (A02.01) | |
| | | STSAVPGSIPLPAVDD | AVPANCLFPA (A02.01) | |
| | | TAAPPEPAPLLTATGS | CLFPAALPST (A02.01) | |
| | | VSLPAAATSAASTLDA | CPTFVSAAA (B07.02) | |
| | | LPAGCVSSAPVSAVPA | FPAALPSTA (B07.02) | |
| | | NCLFPAALPSTAGAIS | FPAALPSTAG (B07.02) | |
| | | RFIWVSGILSPLNDLQ* | GAECHGRPL (B07.02) | |
| | | | GAISRFIWV (A02.01) | |
| | | | ILPAAIPAST (A02.01) | |
| | | | IWVSGILSPL (A24.02) | |
| | | | LLTATGSVSL (A02.01) | |
| | | | LLYTADSSL (A02.01) | |
| | | | LPAAATSAA (B07.02) | |
| | | | LPAAATSAAS (B07.02) | |
| | | | LPAAIPAST (B07.02) | |
| | | | LPAGCVSSA (B07.02) | |
| | | | LPAGCVSSAP (B07.02) | |
| | | | LYTADSSLW (A24.02) | |
| | | | QPAKSSPSA (B07.02) | |
| | | | QPAKSSPSAA (B07.02) | |
| | | | RFIWVSGIL (A24.02) | |
| | | | RPQRVWSTG (B07.02) | |
| | | | RVWSTGPDSI (A02.01) | |
| | | | SAVPGSIPL (B07.02) | |
| | | | SILPAAIPA (A02.01) | |
| | | | SLPAAATSA (A02.01) | |
| | | | SLPAAATSAA (A02.01) | |
| | | | SLWTTRPQR (A03.01) | |
| | | | SLWTTRPQRV | |
| | | | (A02.01) | |
| | | | SPSAAAATL (B07.02) | |
| | | | SPSAAAATLL (B07.02) | |
| | | | TLDALPAGCV | |
| | | | (A02.01) | |
| | | | TVSTTTAPV (A02.01) | |
| | | | VLSASILPA (A02.01) | |
| | | | VLSASILPAA (A02.01) | |
| | | | VPANCLFPA (B07.02) | |
| | | | VPANCLFPAA (B07.02) | |
| | | | VPDPSCPTF (B07.02) | |
| | | | VPGSIPLPA (B07.02) | |
| | | | VPGSIPLPAV (B07.02) | |
| | | | WVSGILSPL (A02.01) | |
| | | | YTADSSLWTT | |
| | | | (A02.01) | |
| | T433fs | PCRAGRRVPWAASLI | APAGMVNRA (B07.02) | STAD, UCEC, BLCA, |
| ARID1A | A441fs | HSRFLLMDNKAPAGM | ASLHRRSYL (B08.01) | BRCA, LUSC, CESC, |
| | Y447fs | VNRARLHITTSKVLTL | ASLHRRSYLK (A03.01) | KIRC, UCS |
| | P483fs | SSSSHPTPSNHRPRPL | FLLMDNKAPA | |
| | P484fs | MPNLRISSSHSLNHHS | (A02.01) | |
| | P504fs | SSPLSLHTPSSHPSLHI | HPRRSPSRL (B07.02, | |
| | S519fs | SSPRLHTPPSSRRHSST | B08.01) | |
| | H544fs | PRASPPTHSHRLSLLTS | HPSLHISSP (B07.02) | |
| | P549fs | SSNLSSQHPRRSPSRL | HRRSYLKIHL (B08.01) | |
| | P554fs | RILSPSLSSPSKLPIPSS | HSRFLLMDNK | |
| | Q563fs | ASLHRRSYLKIHLGLR | (A03.01) | |
| | | HPQPPQ* | KLPIPSSASL (A02.01) | |
| | | | KVLTLSSSSH (A03.01) | |
| | | | LIHSRFLLM (B08.01) | |
| | | | LLMDNKAPA (A02.01) | |
| | | | LMDNKAPAGM | |
| | | | (A02.01) | |
| | | | LPIPSSASL (B07.02) | |
| | | | MPNLRISSS (B07.02, | |
| | | | B08.01) | |
| | | | MPNLRISSSH (B07.02) | |
| | | | NLRISSSHSL (B07.02, | |
| | | | B08.01) | |
| | | | PPTHSHRLSL (B07.02) | |
| | | | RAGRRVPWAA | |
| | | | (B08.01) | |
| | | | RARLHITTSK (A03.01) | |
| | | | RISSSHSLNH (A03.01) | |
| | | | RLHTPPSSR (A03.01) | |
| | | | RLHTPPSSRR (A03.01) | |
| | | | RLRILSPSL (A02.01, | |
| | | | B07.02, B08.01) | |
| | | | RPLMPNLRI (B07.02) | |
| | | | RPRPLMPNL (B07.02) | |
| | | | SASLHRRSYL (B07.02, | |
| | | | B08.01) | |
| | | | SLHISSPRL (A02.01) | |
| | | | SLHRRSYLK (A03.01) | |
| | | | SLHRRSYLKI (B08.01) | |
| | | | SLIHSRFLL (A02.01) | |
| | | | SLIHSRFLLM (A02.01, | |
| | | | B08.01) | |
| | | | SLLTSSSNL (A02.01) | |
| | | | SLNI-IHSSSPL (A02.01, | |
| | | | B07.02, B08.01) | |
| | | | SLSSPSKLPI (A02.01) | |
| | | | SPLSLHTPS (B07.02) | |
| | | | SPLSLHTPSS (B07.02) | |
| | | | SPPTHSHRL (B07.02) | |
| | | | SPRLHTPPS (B07.02) | |
| | | | SPRLHTPPSS (B07.02) | |
| | | | SPSLSSPSKL (B07.02) | |
| | | | SYLKIHLGL (A24.02) | |
| | | | TPSNHRPRPL (B07.02, | |
| | | | B08.01) | |
| | | | TPSSHPSLHI (B07.02) | |
| ARID1A | A2137fs | RTNPTVRMRPHCVPF | CVPFWTGRIL (B07.02) | STAD, UCEC, BLCA, |
| | P2139fs | WTGRILLPSAASVCPIP | HCVPFWTGRIL | BRCA, LUSC, CESC, |
| | L1970fs | FEACHLCQAMTLRCP | (B07.02) | KIRC, UCS |
| | V1994fs | NTQGCCSSWAS* | ILLPSAASV (A02.01) | |
| | | | ILLPSAASVC (A02.01) | |
| | | | LLPSAASVCPI | |
| | | | (A02.01) | |
| | | | LPSAASVCPI (B07.02) | |
| | | | MRPHCVPF (B08.01) | |
| | | | RILLPSAASV (A02.01) | |
| | | | RMRPHCVPF (A24.02, | |
| | | | B07.02, B08.01) | |
| | | | RMRPHCVPFW | |
| | | | (A24.02) | |
| | | | RTNPTVRMR (A03.01) | |
| | | | SVCPIPFEA (A02.01) | |
| | | | TVRMRPHCV (B08.01) | |
| | | | TVRMRPHCVPF | |
| | | | (B08.01) | |
| | | | VPFWTGRIL (B07.02) | |
| | | | VPFWTGRILL (B07.02) | |
| | | | VRMRPHCVPF | |
| | | | (B08.01) | |
| ARID1A | N756fs | TNQALPKIEVICRGTP | AMVPRGVSM (B07.02, | STAD, UCEC, BLCA, |
| | S764fs | RCPSTVPPSPAQPYLR | B08.01) | BRCA, LUSC, CESC, |
| | T783fs | VSLPEDRYTQAWAPT | AMVPRGVSMA | KIRC, UCS |
| | Q799fs | SRTPWGAMVPRGVS | (A02.01) | |
| | A817fs | MAHKVATPGSQTIMP | AWAPTSRTPW | |
| | | CPMPTTPVQAWLEA* | (A24.02) | |
| | | | CPMPTTPVQA (B07.02) | |
| | | | CPSTVPPSPA (B07.02) | |
| | | | GAMVPRGVSM | |
| | | | (B07.02, B08.01) | |
| | | | MPCPMPTTPV (B07.02) | |
| | | | MPTTPVQAW (B07.02) | |
| | | | MPTTPVQAWL | |
| | | | (B07.02) | |
| | | | SLPEDRYTQA (A02.01) | |
| | | | SPAQPYLRV (B07.02) | |
| | | | SPAQPYLRVS (B07.02) | |
| | | | TIMPCPMPT (A02.01) | |
| | | | TPVQAWLEA (B07.02) | |
| | | | TSRTPWGAM (B07.02) | |
| | | | VPPSPAQPYL (B07.02) | |
| | | | VPRGVSMAH (B07.02) | |
| β2M | N62fs | RMERELKKWSIQTCL | CLSARTGLSI (B08.01) | CRC, STAD, SKCM, |
| | E67fs | SARTGLSISCTTLNSPP | CTTLNSPPLK (A03.01) | HNSC |
| | L74fs | LKKMSMPAV* | GLSISCTTL (A02.01) | |
| | F82fs | | SPPLKKMSM (B07.02, | |
| | T91fs | | B08.01) | |
| | E94fs | | TLNSPPLKK (A03.01) | |
| | | | TTLNSPPLK (A03.01) | |
| | | | TTLNSPPLKK (A03.01) | |
| β2M | L13fs | LCSRYSLFLAWRLSSV | LQRFRFTHV (B08.01) | CRC, STAD, SKCM, |
| | S14fs | LQRFRFTHVIQQRMES | LQRFRFTHVI (B08.01) | HNSC |
| | | QIS* | RLSSVLQRF (A24.02) | |
| | | | RLSSVLQRFR (A03.01) | |
| | | | VLQRFRFTHV (A02.01, | |
| | | | B08.01) | |
| CDH1 | A691fs | RSACVTVKGPLASVG | ASVGRHSLSK (A03.01) | ILC LumA Breast |
| | P708fs | RHSLSKQDCKFLPFW | KFLPFWGFL (A24.02) | Cancer |
| | L711fs | GFLEEFLLC* | LASVGRHSL (B07.02) | |
| | | | LPFWGFLEEF (B07.02) | |
| | | | PFWGFLEEF (A24.02) | |
| | | | SVGRHSLSK (A03.01) | |
| CDH1 | H121fs | IQWGTTTAPRPIRPPFL | APRPIRPPF (B07.02) | ILC LumA Breast |
| | P126fs | ESKQNCSHFPTPLLAS | APRPIRPPFL (B07.02) | Cancer |
| | H128fs | EDRRETGLFLPSAAQK | AQKMKKAHFL | |
| | N144fs | MKKAHFLKTWFRSNP | (B08.01) | |
| | V157fs | TKTKKARFSTASLAKE | FLPSAAQKM (A02.01) | |
| | P159fs | LTHPLLVSLLLKEKQD | GLFLPSAAQK (A03.01) | |
| | N166fs | G* | HPLLVSLLL (B07.02) | |
| | N181fs | | KAHFLKTWFR | |
| | F189fs | | (A03.01) | |
| | P201fs | | KARFSTASL (B07.02) | |
| | F205fs | | KMKKAHFLK (A03.01) | |
| | | | KTWFRSNPTK | |
| | | | (A03.01) | |
| | | | LAKELTHPL (B07.02, | |
| | | | B08.01) | |
| | | | LAKELTHPLL (B08.01) | |
| | | | NPTKTKKARF (B07.02) | |
| | | | QKMKKAHFL (B08.01) | |
| | | | RFSTASLAK (A03.01) | |
| | | | RPIRPPFLES (B07.02) | |
| | | | RSNPTKTKK (A03.01) | |
| | | | SLAKELTHPL (A02.01, | |
| | | | B08.01) | |
| | | | TKKARFSTA (B08.01) | |
| CDH1 | V114fs | PTDPFLGLRLGLHLQK | GLRFWNPSR (A03.01) | ILC LumA Breast |
| | P127fs | VFHQSHAEYSGAPPPP | ISQLLSWPQK (A03.01) | Cancer |
| | V132fs | PAPSGLRFWNPSRIAH | RIAHISQLL (A02.01) | |
| | P160fs | ISQLLSWPQKTEERLG | RLGYSSHQL (A02.01) | |
| | | YSSHQLPRK* | SQLLSWPQK (A03.01) | |
| | | | SRIAHISQL (B08.01) | |
| | | | WPQKTEERL (B07.02) | |
| | | | YSSHQLPRK (A03.01) | |
| CDH1 | L731fs | FCCSCCFFGGERWSKS | CPQRMTPGTT (B07.02) | ILC LumA Breast |
| | R749fs | PYCPQRMTPGTTFITM | EAEKRTRTL (B08.01) | Cancer |
| | E757fs | MKKEAEKRTRTLT* | GTTFITMMK (A03.01) | |
| | G759fs | | GTTFITMMKK | |
| | | | (A03.01) | |
| | | | ITMMKKEAEK | |
| | | | (A03.01) | |
| | | | RMTPGTTFI (A02.01) | |
| | | | SPYCPQRMT (B07.02) | |
| | | | TMMKKEAEK (A03.01) | |
| | | | TPGTTFITM (B07.02) | |
| | | | TPGTTFITMM (B07.02) | |
| | | | TTFITMMKK (A03.01) | |
| CDH1 | S19fs | WRRNCKAPVSLRKSV | CPGATWREA (B07.02) | ILC LumA Breast |
| | E24fs | QTPARSSPARPDRTRR | CPGATWREAA | Cancer |
| | S36fs | LPSLGVPGQPWALGA | (B07.02) | |
| | | AASRRCCCCCRSPLGS | RSRCPGATWR | |
| | | ARSRSPATLALTPRAT | (A03.01) | |
| | | RSRCPGATWREAASW | TPRATRSRC (B07.02) | |
| | | AE* | | |
| GATA3 | P394fs | PGRPLQTHVLPEPHLA | HVLPEPHLAL (B07.02) | Breast Cancer |
| | P387fs | LQPLQPHADHAHADA | RPLQTHVLPE (B07.02) | |
| | S398fs | PAIQPVLWTTPPLQHG | VLWTTPPLQH | |
| | H400fs | HRHGLEPCSMLTGPP | (A03.01) | |
| | M401fs | ARVPAVPFDLHFCRSS | | |
| | S408fs | IMKPKRDGYMFLKAE | | |
| | P409fs | SKIMFATLQRSSLWCL | | |
| | S408fs | CSNH* | | |
| | P409fs | | | |
| | T419fs | | | |
| | H424fs | | | |
| | P425fs | | | |
| | S427fs | | | |
| | F431fs | | | |
| | S430fs | | | |
| | H434fs | | | |
| | H435fs | | | |
| | S438fs | | | |
| | M443fs | | | |
| | G444fs | | | |
| | *445fs | | | |
| GATA3 | P426fs | PRPRRCTRHPACPLDH | APSESPCSPF (B07.02) | Breast Cancer |
| | H434fs | TTPPAWSPPWVRALL | CPLDHTTPPA (B07.02) | |
| | P433fs | DAHRAPSESPCSPFRL | FLQEQYHEA (A02.01, | |
| | T441fs | AFLQEQYHEA* | B08.01) | |
| | | | RLAFLQEQYH | |
| | | | (A03.01) | |
| | | | SPCSPFRLAF (B07.02) | |
| | | | SPPWVRALL (B07.02) | |
| | | | YPACPLDHTT (B07.02) | |
| MLL2 | P519fs | TRRCHCCPHLRSHP | CPALHLRSCPC (B08.01) | STAD, BLCA, CRC, |
| | E524fs | HHLRNHPRPHHLRHH | CLHHRRHLV (B08.01) | HNSC, BRCA |
| | P647fs | ACHHHLRNCPHPHFL | CLEIHRRHLVC | |
| | S654fs | RHCTCPGRWRNRPSL | (B08.01) | |
| | L656fs | RRLRSLLCLPHLNHHL | CLHRKSHPHL (B08.01) | |
| | R755fs | FLHWRSRPCLHRKSH | CLRSHACPP (B08.01) | |
| | L761fs | PHLLHLRRLYPHHLK | CLRSHTCPP (B08.01) | |
| | Q773fs | HRPCPHHLKNLLCPR | CLWCHACLH (A03.01) | |
| | | HLRNCPLPRHLKHLA | CPHHLKNHL (B07.02) | |
| | | CLHHLRSHPCPLHLKS | CPHHLKNLL (B07.02) | |
| | | HPCLHHRRHLVCSHH | CPHHLRTRL (B07.02, | |
| | | LKSLLCPLHLRSLPFP | B08.01) | |
| | | HHLRHHACPHHLRTR | CPLHLRSLPF (B07.02, | |
| | | LCPHHLKNHLCPPHLR | B08.01) | |
| | | YRAYPPCLWCHACLH | CPLPRHLKHL (B07.02, | |
| | | RLRNLPCPHRLRSLPR | B08.01) | |
| | | PLHLRLHASPHHLRTP | CPLSLRSHPC (B07.02) | |
| | | PHPHHLRTHLLPHHRR | CPRHLRNCPL (B07.02, | |
| | | TRSCPCRWRSHPCCH | B08.01) | |
| | | YLRSRNSAPGPRGRTC | FPHHLRHHA (B07.02, | |
| | | HPGLRSRTCPPGLRSH | B08.01) | |
| | | TYLRRLRSHTCPPSLR | FPFHLRHHAC (B07.02, | |
| | | SHAYALCLRSHTCPPR | B08.01) | |
| | | LRDHICPLSLRNCTCP | GLRSRTCPP (B08.01) | |
| | | PRLRSRTCLLCLRSHA | HACLHRLRNL | |
| | | CPPNLRNHTCPPSLRS | (B08.01) | |
| | | HACPPGLRNRICPLSL | HLACLHHLR (A03.01) | |
| | | RSHPCPLGLKSPLRSQ | HLCPPHLRY (A03.01) | |
| | | ANALHLRSCPCSLPLG | HLCPPHLRYR (A03.01) | |
| | | NHPYLPCLESQPCLSL | HLKHLACLH (A03.01) | |
| | | GNHLCPLCPRSCRCPH | HLKHRPCPH (B08.01) | |
| | | LGSHPCRLS* | HLKNHLCPP (B08.01) | |
| | | | HLKSHPCLH (A03.01) | |
| | | | HLKSLLCPL (A02.01, | |
| | | | B08.01) | |
| | | | HLLHLRRLY (A03.01) | |
| | | | HLRNCPLPR (A03.01) | |
| | | | HLRNCPLPRH (A03.01) | |
| | | | HLRRLYPHHL (B08.01) | |
| | | | HLRSHPCPL (B07.02, | |
| | | | B08.01) | |
| | | | HLRSHPCPLH (A03.01) | |
| | | | HLRSLPFPH (A03.01) | |
| | | | HLRTRLCPH (A03.01, | |
| | | | B08.01) | |
| | | | HLVCSHHLK (A03.01) | |
| | | | HPCLHHRRHL (B07.02, | |
| | | | B08.01) | |
| | | | HPGLRSRTC (B07.02) | |
| | | | HPHLLHLRRL (B07.02, | |
| | | | B08.01) | |
| | | | HRKSHPHLL (B08.01) | |
| | | | HRRTRSCPC (B08.01) | |
| | | | KSHPHLLHLR (A03.01) | |
| | | | KSLLCPLHLR (A03.01) | |
| | | | LLCPLHLRSL (A02.01, | |
| | | | B08.01) | |
| | | | LLHLRRLYPH (B08.01) | |
| | | | LPRHLKHLA (B07.02) | |
| | | | LPRHLKHLAC (B07.02, | |
| | | | B08.01) | |
| | | | LRRLRSHTC (B08.01) | |
| | | | LRRLYPHHL (B08.01) | |
| | | | LVCSHHLKSL (B08.01) | |
| | | | NLRNHTCPPS (B08.01) | |
| | | | PLHLRSLPF (B08.01) | |
| | | | RLCPHHLKNH | |
| | | | (A03.01) | |
| | | | RLYPHHLKH (A03.01) | |
| | | | RLYPHHLKHR | |
| | | | (A03.01) | |
| | | | RPCPHHLKNL (B07.02) | |
| | | | RSHPCPLHLK (A03.01) | |
| | | | RSLPFPHHLR (A03.01) | |
| | | | RTRLCPHHL (B07.02) | |
| | | | RTRLCPHHLK (A03.01) | |
| | | | SLLCPLHLR (A03.01) | |
| | | | SLRSHACPP (B08.01) | |
| | | | SPLRSQANA (B07.02) | |
| | | | YLRRLRSHT (B08.01) | |
| | | | YPHHLKHRPC (B07.02, | |
| | | | B08.01) | |
| PTEN | I122fs | SWKGTNWCNDMCIFI | FITSGQIFK (A03.01) | UCEC, PRAD, |
| | I135fs | TSGQIFKGTRGPRFLW | IFITSGQIF (A24.02) | SKCM, STAD, |
| | A148fs | GSKDQRQKGSNYSQS | SQSEALCVL (A02.01) | BRCA, LUSC, KIRC, |
| | L152fs | EALCVLL* | SQSEALCVLL (A02.01) | LIHC, KIRP, GBM |
| | D162fs | | | |
| | I168fs | | | |
| PTEN | L265fs | KRTKCFTFG* | | UCEC, PRAD, |
| | K266fs | | | SKCM, STAD, |
| | | | | BRCA, LUSC, KIRC, |
| | | | | LIHC, KIRP, GBM |
| PTEN | A39fs | PIFIQTLLLWDFLQKD | AYTGTILMM (A24.02) | UCEC, PRAD, |
| | E40fs | LKAYTGTILMM* | DLKAYTGTIL (B08.01) | SKCM, STAD, |
| | V45fs | | | BRCA, LUSC, KIRC, |
| | R47fs | | | LIHC, KIRP, GBM |
| | N48fs | | | |
| PTEN | T319fs | QKMILTKQIKTKPTDT | ILTKQIKTK (A03.01) | UCEC, PRAD, |
| | T321fs | FLQILR* | KMILTKQIK (A03.01) | SKCM, STAD, |
| | K327fs | | KPTDTFLQI (B07.02) | BRCA, LUSC, KIRC, |
| | A328fs | | KPTDTFLQIL (B07.02) | LIHC, KIRP, GBM |
| | A333fs | | MILTKQIKTK (A03.01) | |
| PTEN | N63fs | GFWIQSIKTITRYTIFV | ITRYTIFVLK (A03.01) | UCEC, PRAD, |
| | E73fs | LKDIMTPPNLIAELHNI | LIAELHNIL (A02.01) | SKCM, STAD, |
| | A86fs | LLKTITHHS* | LIAELHNILL (A02.01) | BRCA, LUSC, KIRC, |
| | N94fs | | MTPPNLIAEL (A02.01) | LIHC, KIRP, GBM |
| | | | NLIAELHNI (A02.01) | |
| | | | NLIAELHNIL (A02.01) | |
| | | | RYTIFVLKDI (A24.02) | |
| | | | TITRYTIFVL (A02.01) | |
| | | | TPPNLIAEL (B07.02) | |
| PTEN | T202fs | NYSNVQWRNLQSSVC | FLQFRTHTT (A02.01, | UCEC, PRAD, |
| | G209fs | GLPAKGEDIFLQFRTH | B08.01) | SKCM, STAD, |
| | C211fs | TTGRQVHVL* | LPAKGEDIFL (B07.02) | BRCA, LUSC, KIRC, |
| | I224fs | | LQFRTHTTGR (A03.01) | LIHC, KIRP, GBM |
| | G230fs | | NLQSSVCGL (A02.01) | |
| | P231fs | | SSVCGLPAK (A03.01) | |
| | R233fs | | VQWRNLQSSV | |
| | D236fs | | (A02.01) | |
| PTEN | G251fs | YQSRVLPQTEQDAKK | GQNVSLLGK (A03.01) | UCEC, PRAD, |
| | E256fs | GQNVSLLGKYILHTRT | HTRTRGNLRK | SKCM, STAD, |
| | K260fs | RGNLRKSRKWKSM* | (A03.01) | BRCA, LUSC, KIRC, |
| | Q261fs | | ILHTRTRGNL (B08.01) | LIHC, KIRP, GBM |
| | L265fs | | KGQNVSLLGK | |
| | M270fs | | (A03.01) | |
| | H272fs | | LLGKYILHT (A02.01) | |
| | T286fs | | LRKSRKWKSM | |
| | E288fs | | (B08.01) | |
| | | | SLLGKYILH (A03.01) | |
| | | | SLLGKYILHT (A02.01) | |
| TP53 | A70fs | SSQNARGCSPRGPCTS | CTSPLLAPV (A02.01) | BRCA, CRC, LUAD, |
| | P72fs | SSYTGGPCTSPLLAPVI | FPENLPGQL (B07.02) | PRAD, HNSC, LUSC, |
| | A76fs | FCPFPENLPGQLRFPS | GLLAFWDSQV | PAAD, STAD, BLCA, |
| | A79fs | GLLAFWDSQVCDLHV | (A02.01) | OV, LIHC, SKCM, |
| | P89fs | LPCPQQDVLPTGQDLP | IFCPFPENL (A24.02) | UCEC, LAML, UCS, |
| | W91fs | CAAVG* | LLAFWDSQV (A02.01) | KICH, GBM, ACC |
| | S96fs | | LLAPVIFCP (A02.01) | |
| | V97fs | | LLAPVIFCPF (A02.01, | |
| | V97fs | | A24.02) | |
| | G108fs | | LPCPQQDVL ( B07.02) | |
| | G117fs | | RFPSGLLAF ( A24.02) | |
| | S121fs | | RFPSGLLAFW (A24.02) | |
| | V122fs | | SPLLAPVIF (B07.02) | |
| | C124fs | | SPRGPCTSS (B07.02) | |
| | K139fs | | SPRGPCTSSS (B07.02) | |
| | V143fs | | SQVCDLHVL (A02.01) | |
| | | | VIFCPFPENL (A02.01) | |
| TP53 | V173fs | GAAPTMSAAQIAMV | AMVWPLLSI (A02.01) | BRCA, CRC, LUAD, |
| | H178fs | WPLLSILSEWKEICVW | AMVWPLLSIL (A02.01) | PRAD, HNSC, LUSC, |
| | D186fs | SIWMTETLFDIVWWC | AQIAMVWPL (A02.01, | PAAD, STAD, BLCA, |
| | H193fs | PMSRLRLALTVPPSTT | A24.02) | OV, LIHC, SKCM, |
| | L194fs | TTCVTVPAWAA* | AQIAMVWPLL | UCEC, LAML, UCS, |
| | E198fs | | (A02.01) | KICH, GBM, ACC |
| | V203fs | | CPMSRLRLA (B07.02, | |
| | E204fs | | B08.01) | |
| | L206fs | | CPMSRLRLAL (B07.02, | |
| | D207fs | | B08.01) | |
| | N210fs | | IAMVWPLLSI (A02.01, | |
| | T211fs | | A24.02, B08.01) | |
| | F212fs | | ILSEWKEICV (A02.01) | |
| | V225fs | | IVWWCPMSR (A03.01) | |
| | S241fs | | IVWWCPMSRL | |
| | | | (A02.01) | |
| | | | IWMTETLFDI (A24.02) | |
| | | | LLSILSEWK (A03.01) | |
| | | | MSAAQIAMV (A02.01) | |
| | | | MSRLRLALT (B08.01) | |
| | | | MSRLRLALTV (B08.01) | |
| | | | MVWPLLSIL (A02.01) | |
| | | | RLALTVPPST (A02.01) | |
| | | | TLFDIVWWC (A02.01) | |
| | | | TLFDIVWWCP | |
| | | | (A02.01) | |
| | | | TMSAAQIAMV | |
| | | | (A02.01) | |
| | | | VWSIWMTETL | |
| | | | (A24.02) | |
| | | | WMTETLFDI (A02.01, | |
| | | | A24.02) | |
| | | | WMTETLFDIV (A01.01, | |
| | | | A02.01) | |
| TP53 | R248fs | TGGPSSPSSHWKTPVV | ALRCVFVPV (A02.01, | BRCA, CRC, LUAD, |
| | P250fs | IYWDGTALRCVFVPV | B08.01) | PRAD, HNSC, LUSC, |
| | S260fs | LGETGAQRKRISARK | ALRCVFVPVL (A02.01, | PAAD, STAD, BLCA, |
| | N263fs | GSLTTSCPQGALSEHC | B08.01) | OV, LIHC, SKCM, |
| | G266fs | PTTPAPLPSQRRNHW | ALSEHCPTT (A02.01) | UCEC, LAML, UCS, |
| | N268fs | MENISPFRSVGVSASR | AQRKRISARK (A03.01) | KICH, GBM, ACC |
| | V272fs | CSES* | GAQRKRISA (B08.01) | |
| | V274fs | | HWMENISPF (A24.02) | |
| | P278fs | | LPSQRRNHW (B07.02) | |
| | D281fs | | LPSQRRNHWM | |
| | R282fs | | (B07.02, B08.01) | |
| | T284fs | | NISPFRSVGV (A02.01) | |
| | E285fs | | RISARKGSL (B07.02, | |
| | L289fs | | B08.01) | |
| | K292fs | | SPFRSVGVSA (B07.02) | |
| | P301fs | | SPSSHWKTPV (B07.02, | |
| | S303fs | | B08.01) | |
| | T312fs | | TALRCVFVPV (A02.01) | |
| | S314fs | | VIYWDGTAL (A02.01) | |
| | K319fs | | VIYWDGTALR | |
| | K320fs | | (A03.01) | |
| | P322fs | | VLGETGAQRK | |
| | Y327fs | | (A03.01) | |
| | F328fs | | | |
| | L330fs | | | |
| | R333fs | | | |
| | R335fs | | | |
| | R337fs | | | |
| | E339fs | | | |
| TP53 | S149fs | FHTPARHPRPRHGHL | HPRPRHGHL (B07.02, | BRCA, CRC, LUAD, |
| | P151fs | QAVTAHDGGCEALPP | B08.01) | PRAD, HNSC, LUSC, |
| | P152fs | P* | HPRPRHGHLQ | PAAD, STAD, BLCA, |
| | V157fs | | (B07.02) | OV, LIHC, SKCM, |
| | Q165fs | | RPRHGHLQA (B07.02) | UCEC, LAML, UCS, |
| | S166fs | | RPRHGHLQAV | KICH, GBM, ACC |
| | H168fs | | (B07.02, B08.01) | |
| | V173fs | | | |
| TP53 | P47fs | CCPRTILNNGSLKTQV | GSLKTQVQMK | BRCA, CRC, LUAD, |
| | D48fs | QMKLPECQRLLPPWP | (A03.01) | PRAD, HNSC, LUSC, |
| | D49fs | LHQQLLHRRPLHQPPP | PPGPCHLLSL (B07.02) | PAAD, STAD, BLCA, |
| | Q52fs | GPCHLLSLPRKPTRAA | RTILNNGSLK (A03.01) | OV, LIHC, SKCM, |
| | F54fs | TVSVWASCILGQPSL* | SLKTQVQMK (A03.01) | UCEC, LAML, UCS, |
| | E56fs | | SLKTQVQMKL | KICH, GBM, ACC |
| | P58fs | | (B08.01) | |
| | P60fs | | TILNNGSLK (A03.01) | |
| | E62fs | | | |
| | M66fs | | | |
| | P72fs | | | |
| | V73fs | | | |
| | P75fs | | | |
| | A78fs | | | |
| | P82fs | | | |
| | P85fs | | | |
| | S96fs | | | |
| | P98fs | | | |
| | T102fs | | | |
| | Y103fs | | | |
| | G108fs | | | |
| | F109fs | | | |
| | R110fs | | | |
| | G117fs | | | |
| TP53 | L26fs | VRKHFQTYGNYFLKT | CPPCRPKQWM | BRCA, CRC, LUAD, |
| | P27fs | TFCPPCRPKQWMI* | (B07.02) | PRAD, HNSC, LUSC, |
| | P34fs | | TTFCPPCRPIK (A03.01) | PAAD, STAD, BLCA, |
| | P36fs | | | OV, LIHC, SKCM, |
| | A39fs | | | UCEC, LAML, UCS, |
| | Q38fs | | | KICH, GBM, ACC |
| TP53 | C124fs | LARTPLPSTRCFANWP | CFANWPRPAL | BRCA, CRC, LUAD, |
| | L130fs | RPALCSCGLIPHPRPAP | (A24.02) | PRAD HNSC LUSC, |
| | N131fs | ASAPWPSTSSHST* | FANWPRPAL (B07.02, | PAAD, STAD, BLCA, |
| | C135fs | | B08.01) | OV, LIHC, SKCM, |
| | K139fs | | GLIPHPRPA (A02.01) | UCEC, LAML, UCS, |
| | A138fs | | HPRPAPASA (B07.02, | KICH, GBM, ACC |
| | T140fs | | B08.01) | |
| | V143fs | | HPRPAPASAP (B07.02) | |
| | Q144fs | | IPHPRPAPA (B07.02, | |
| | V147fs | | B08.01) | |
| | T150fs | | IPHPRPAPAS (B07.02) | |
| | P151fs | | RPALCSCGL (B07.02) | |
| | P152fs | | RPALCSCGLI (B07.02) | |
| | G154fs | | TPLPSTRCF (B07.02) | |
| | R156fs | | WPRPALCSC (B07.02) | |
| | R158fs | | WPRPALCSCG | |
| | A161fs | | (B07.02) | |
| VHL | L178fs | ELQETGHRQVALRRS | ALRRSGRPPK (A03.01) | KIRC, KIRP |
| | D179fs | GRPPKCAERPGAADT | GLVPSLVSK (A03.01) | |
| | L184fs | GAHCTSTDGRLKISVE | KISVETYTV (A02.01) | |
| | T202fs | TYTVSSQLLMVLMSL | LLMVLMSLDL | |
| | R205fs | DLDTGLVPSLVSKCLI | (A02.01, B08.01) | |
| | D213fs | LRVK* | LMSLDLDTGL | |
| | G212fs | | (A02.01) | |
| | | | LMVLMSLDL (A02.01) | |
| | | | LVSKCLILRV (A02.01) | |
| | | | QLLMVLMSL (A02.01, | |
| | | | B08.01) | |
| | | | RPGAADTGA (B07.02) | |
| | | | RPGAADTGAH | |
| | | | (B07.02) | |
| | | | SLDLDTGLV (A02.01) | |
| | | | SLVSKCLIL (A02.01, | |
| | | | B08.01) | |
| | | | SQLLMVLMSL | |
| | | | (A02.01) | |
| | | | TVSSQLLMV (A02.01) | |
| | | | TYTVSSQLL (A24.02) | |
| | | | TYTVSSQLLM (A24.02) | |
| | | | VLMSLDLDT (A02.01) | |
| | | | VPSLVSKCL (B07.02) | |
| | | | VSKCLILRVK (A03.01) | |
| | | | YTVSSQLLM (A01.01) | |
| | | | YTVSSQLLMV | |
| | | | (A02.01) | |
| VHL | L158fs | KSDASRLSGA* | | KIRC, KIRP |
| | K159fs | | | |
| | R161fs | | | |
| | Q164fs | | | |
| VHL | P146fs | RTAYFCQYHTASVYS | FCQYHTASV (B08.01) | KIRC, KIRP |
| | I147fs | ERAMPPGCPEPSQA* | | |
| | F148fs | | | |
| | L158fs | | | |
| VHL | S68fs | TRASPPRSSSAIAVRAS | CPYGSTSTA (B07.02) | KIRC, KIRP |
| | S72fs | CCPYGSTSTASRSPTQ | CPYGSTSTAS (B07.02) | |
| | I75fs | RCRLARAAASTATEV | LARAAASTAT (B07.02) | |
| | S80fs | TFGSSEMQGHTMGFW | MLTDSLFLP (A02.01) | |
| | P86fs | LTKLNYLCHLSMLTD | PPRSSSAIAV (B07.02) | |
| | P97fs | SLFLPISHCQCIL* | RAAASTATEV (B07.02) | |
| | I109fs | | SPPRSSSAI (B07.02) | |
| | H115fs | | SPPRSSSAIA (B07.02) | |
| | L116fs | | SPTQRCRLA (B07.02) | |
| | G123fs | | TQRCRLARA (B08.01) | |
| | T124fs | | TQRCRLARAA | |
| | N131fs | | (B08.01) | |
| | L135fs | | | |
| | V137fs | | | |
| | G144fs | | | |
| | D143fs | | | |
| | I147fs | | | |
| VHL | K171fs | SSLRITGDWTSSGRST | KIWKTTQMCR | KIRC, KIRP |
| | P172fs | KIWKTTQMCRKTWSG | (A03.01) | |
| | N174fs | * | WTSSGRSTK (A03.01) | |
| | L178fs | | | |
| | D179fs | | | |
| | L188fs | | | |
| VHL | V62fs | RRRRGGVGRRGVRPG | ALGELARAL (A02.01) | KIRC, KIRP |
| | V66fs | RVRPGGTGRRGGDGG | AQLRRRAAA (B08.01) | |
| | Q73fs | RAAAARAALGELARA | AQLRRRAAAL | |
| | V84fs | LPGHLLQSQSARRAA | (B08.01) | |
| | F91fs | RMAQLRRRAAALPNA | ARRAARMAQL | |
| | T100fs | AAWHGPPHPQLPRSP | (B08.01) | |
| | P103fs | LALQRCRDTRWASG* | HPQLPRSPL (B07.02, | |
| | S111fs | | B08.01) | |
| | L116fs | | HPQLPRSPLA (B07.02) | |
| | H115fs | | LARALPGHL (B07.02) | |
| | D126fs | | LARALPGHLL (B07.02) | |
| | | | MAQLRRRAA (B07.02, | |
| | | | B08.01) | |
| | | | MAQLRRRAAA | |
| | | | (B07.02, B08.01) | |
| | | | QLRRRAAAL (B07.02, | |
| | | | B08.01) | |
| | | | RAAALPNAAA | |
| | | | (B07.02) | |
| | | | RMAQLRRRAA | |
| | | | (B07.02, B08.01) | |
| | | | SQSARRAARM | |
| | | | (B08.01) | |

TABLE 35D

| | | CRYPTIC EXON ¹ | | |
|---|---|---|---|---|
| AR-v7 | cryptic final | SCKVFFKRAAEGKQK | GMTLGEKFRV | Prostate Cancer, |
| | exon | YLCASRNDCTIDKFRR | (A02:01) | Castration-resistant |
| | | KNCPSCRLRKCYEAG | RVGNCKHLK (A03.01) | Prostate Cancer |
| | | MTLGEKFRVGNCKHL | | |
| | | KMTRP* | | |

TABLE 35E

| | | OUT OF FRAME | | |
|---|---|---|---|---|
| | | FUSIONS ^{1,3} | | |
| AC01199 | AC011997.1:L | MAGAPPPASLPPCSLIS | | |
| 7.1:LRRC | RRC69 | DCCASNQRDSVGVGP | GPSEPGNNI (B07.02) | LUSC, Breast Cancer, |
| 69 | *out-of-frame | SEP:G:NNIKICNESAS | KICNESASRK (A03.01) | Head and Neck |
| | | RK* | | Cancer, LUAD |
| EEF1DP3 | EEF1DP3:FR | HGWRPFLPVRARSRW | GIQVLNVSLK (A03.01) | Breast Cancer |
| | Y *out-of- | NRRLDVTVANGR:S:W | IQVLNVSLK (A03.01) | |
| | frame | KYGWSLLRVPQVNG | KSSSNVISY (A01.01, | |
| | | IQVLNVSLKSSSNVIS | A03.01) | |
| | | YE* | KYGWSLLRV (A24.02) | |
| | | | RSWKYGWSL (A02.01) | |
| | | | SLKSSSNVI (B08.01) | |
| | | | SWKYGWSLL (A24.02) | |
| | | | TVANGRSWK (A03.01) | |
| | | | VPQVNGIQV (B07.02) | |
| | | | VPQVNGIQVL (B07.02) | |
| | | | VTVANGRSWK | |
| | | | (A03.01) | |
| | | | WSLLRVPQV (B08.01) | |
| MAD1L1: | MAD1L1:MA | RLKEVFQTKIQEFRKA | HPGDCLIFKL (B07.02) | CLL |
| MAFK | FK | CYTLTGYQIDITTENQ | KLRVPGSSV (B07.02) | |
| | | YRLTSLYAEHPGDCLI | KLRVPGSSVL (B07.02) | |
| | | FK::LRVPGSSVLVTV | RVPGSSVLV (A02.01) | |
| | | PGL* | SVLVTVPGL (A02.01) | |
| | | | VPGSSVLVTV (B07.02) | |
| PPP1R1B | PPP1R1B:ST | AEVLKVIRQSAGQKT | ALLLRPRPPR (A03.01) | Breast Cancer |
| :STARD3 | ARD3 | TCGQGLEGPWERPPPL | ALSALLLRPR (A03.01) | |
| | | DESERDGGSEDQVED | | |
| | | PALS:A:LLLRPRPPRP | | |
| | | EVGAHQDEQAAQGA | | |
| | | DPRLGAQPACRGLP | | |
| | | GLLTVPQPEPLLAPP | | |
| | | SAA* | | |

Table 35F

| | | IN FRAME | | |
|---|---|---|---|---|
| | | DELETIONS and | | |
| | | FUSIONS ^{1,2} | | |
| BCR:ABL | BCR:ABL | ERAEWRENIREQQKK | LTINKEEAL (A02.01, | CML, AML |
| | | CFRSFSLTSVELQMLT | B08.01) | |
| | | NSCVKLQTVHSIPLTI | | |
| | | NKE::EALQRPVASDF | | |
| | | EPQGLSEAARWNSK | | |
| | | ENLLAGPSENDPNLF | | |
| | | VALYDFVASG | | |
| BCR:ABL | BCR:ABL | ELQMLTNSCVKLQTV | IVHSATGFK (A03.01) | CML, AML |
| | | HSIPLTINKEDDESPGL | ATGFKQSSK (A03.01) | |
| | | YGFLNVIVHSATGFKQ | | |
| | | SS:K:ALQRPVASDFE | | |
| | | PQGLSEAARWNSKE | | |
| | | NLLAGPSENDPNLFV | | |
| | | ALYDFVASGD | | |
| C11orf95: | C11orf95:REL | ISNSWDAHLGLGACG | ELFPLIFPA (A02.01, | Supretentorial |
| RELA | A | EAEGLGVQGAEEEEE | B08.01) | ependyomas |
| | | EEEEEEEEGAGVPACP | KGPELFPLI (A02.01, | |
| | | PKGP:E:LFPLIFPAEP | A24.02) | |
| | | AQASGPYVEIIEQPK | KGPELFPLIF (A24.02) | |
| | | QRGMRFRYKCEGRS | | |
| | | AGSIPGERSTD | | |
| CBFB:M | (variant “type | LQRLDGMGCLEFDEE | | AML |
| YH11 | a”) | RAQQEDALAQQAFEE | | |
| | | ARRRTREFEDRDRSH | | |
| | | REEME::VHELEKSKR | | |
| | | ALETQMEEMKTQLE | | |
| | | ELEDELQATEDAKL | | |
| | | RLEVNMQALKGQF | | |
| CD74:RO | (exon6:exon32) | KGSFPENLRHLKNTM | KPTDAPPKAGV | NSCLC, Crizotinib |
| S1 | | ETIDWKVFESWMHH | (B07.02) | resistance |
| | | WLLFEMSRHSLEQKP | | |
| | | TDAPPK::AGVPNKPG | | |
| | | IPKLLEGSKNSIQWE | | |
| | | KAEDNGCRITYYILEI | | |
| | | RKSTSNNLQNQ | | |
| EML4:ALK | EML4:ALK | SWENSDDSRNKLSKIP | QVYRRKHQEL | NSCLC |
| | | STPKLIPKVTKTADKH | (B08.01) | |
| | | KDVIINQAKMSTREK | STREKNSQV (B08.01) | |
| | | NSQ:V:YRRKHQELQ | VYRRKHQEL (A24.02, | |
| | | AMQMELQSPEYKLS | B08.01) | |
| | | KLRTSTIMTDYNPNY | | |
| | | CFAGKTSSISDL | | |
| FGFR3:T | FGFR3:TACC | EGHRMDKPANCTHDL | VLTVTSTDV (A02.01) | Bladder Cancer, |
| ACC3 | 3 | YMIMRECWHAAPSQR | VLTVTSTDVK (A03.01) | LUSC |
| | | PTFKQLVEDLDRVLT | | |
| | | VTSTD::VKATQEENR | | |
| | | ELRSRCEELHGKNLE | | |
| | | LGKIMDRFEEVVYQ | | |
| | | AMEEVQKQKELS | | |
| NAB:ST | NAB:STAT6 | RDNTLLLRRVELFSLS | IMSLWGLVS (A02.01) | Solitary fibrous |
| AT6 | “” | RQVARESTYLSSLKGS | IMSLWGLVSK | tumors |
| | | RLHPEELGGPPLKKLK | (A03.01) | |
| | | QE::ATSKSQIMSLWG | KLKQEATSK (A03.01) | |
| | | LVSKMPPEKVQRLY | QIMSLWGLV (A02.01) | |
| | | VDFPQHLRHLLGDW | SQIMSLWGL (A02.01, | |
| | | LESQPWEFLVGSDAF | A24.02, B08.01) | |
| | | CC | SQIMSLWGLV | |
| | | | (A02.01) | |
| | | | TSKSQIMSL (B08.01) | |
| NDRG1:E | NDRG1:ERG | MSREMQDVDLAEVKP | LLQEFDVQEA (A02.01) | Prostate Cancer |
| RG | | LVEKGETITGLLQEFD | LQEFDVQEAL (A02.01) | |
| | | VQ::EALSVVSEDQSL | | |
| | | FECAYGTPHLAKTE | | |
| | | MTASSSSDYGQTSK | | |
| | | MSPRVPQQDW | | |
| PML:RA | PML:RARA | VLDMHGFLRQALCRL | | Acute promyelocytic |
| RA | (exon3:exon3) | RQEEPQSLQAAVRTD | | leukemia |
| | | GFDEFKVRLQDLSSCI | | |
| | | TQGK:A:IETQSSSSEE | | |
| | | IVPSPPSPPPLPRIYKP | | |
| | | CFVCQDKSSGYHYG | | |
| | | VSACEGCKG | | |
| PML:RA | PML:RARA | RSSPEQPRPSTSKAVSP | | Acute promyelocytic |
| RA | (exon6:exon3) | PHLDGPPSPRSPVIGSE | | leukemia |
| | | VFLPNSNHVASGAGE | | |
| | | A:A:IETQSSSSEEIVPS | | |
| | | PPSPPPLPRIYKPCFV | | |
| | | CQDKSSGYHYGVSA | | |
| | | CEGCKG | | |
| RUNX1 | RUNX1(ex5)- | VARFNDLRFVGRSGR | GPREPRNRT (B07.02) | AML |
| | RUNX1T1(ex | GKSFTLTITVFTNPPQ | RNRTEKHSTM | |
| | 2) | VATYHRAIKITVDGPR | (B08.01) | |
| | | EPR:N:RTEKHSTMPD | | |
| | | SPVDVKTQSRLTPPT | | |
| | | MPPPPTTQGAPRTSS | | |
| | | FTPTTLTNGT | | |
| TMPRSS | TMPRSS2:ER | MALNS::EALSVVSED | ALNSEALSV (A02.01) | Prostate Cancer |
| 2:ERG | G | QSLFECAYGTPHLAKT | ALNSEALSVV (A02.01) | |
| | | EMTASSSSDYGQTSK | MALNSEALSV | |
| | | MSPRVPQQDW | (A02.01, B08.01) | |

¹ Underlined AAs represent non-native AAs
² Bolded AAs represent native AAs of the amino acid sequence encoded by the second of the two fused genes
³ Bolded and underlined AAs represent non-native AAs of the amino acid sequence encoded by the second of the two fused genes due to a frameshift.

Table 36 below provides a list of selected HLA-restricted BTK peptides for the purpose of this Application and the corresponding protein encoded by the HLA allele to which the mutant BTK peptide binds or is predicted to bind.

TABLE 36

| BTK PEPTIDE | HLA allele |
|---|---|
| SLLNYLREM | HLA-A02:04 |
| | HLA-A02:03 |
| | HLA-C03:02 |
| | HLA-A03:01 |
| | HLA-A32:01 |
| | HLA-A02:07 |
| | HLA-C14:03 |
| | HLA-C14:02 |
| | HLA-A31:01 |
| | HLA-A30:02 |
| | HLA-A74:01 |
| | HLA-C06:02 |
| | HLA-B15:03 |
| | HLA-B46:01 |
| | HLA-B13:02 |
| | HLA-A25:01 |
| | HLA-A29:02 |
| | HLA-C01:02 |
| EYMANGSLL | HLA-C14:02 |
| | HLA-C14:03 |
| | HLA-A33:03 |
| | HLA-C04:01 |
| | HLA-B15:09 |
| | HLA-B38:01 |
| TEYMANGSL | HLA-B14:02 |
| | HLA-B49:01 |
| | HLA-B44:03 |
| | HLA-B44:02 |
| | HLA-B37:01 |
| | HLA-B15:09 |
| | HLA-B41:01 |
| | HLA-B50:01 |
| MANGSLLNY | HLA-C02:02 |
| | HLA-C03:02 |
| | HLA-B53:01 |
| | HLA-C12:02 |
| | HLA-C12:03 |
| | HLA-A36:01 |
| | HLA-A26:01 |
| | HLA-A25:01 |
| | HLA-B57:01 |
| | HLA-A03:01 |
| | HLA-B46:01 |
| | HLA-B15:03 |
| | HLA-A33:03 |
| | HLA-B35:03 |
| | HLA-A11:01 |
| YMANGSLLNY | HLA-A29:02 |
| | HLA-A36:01 |
| | HLA-B46:01 |
| | HLA-A25:01 |
| | HLA-B15:01 |
| | HLA-A26:01 |
| | HLA-A30:02 |
| | HLA-A32:01 |

Table 37 provides a list of selected BTK peptides and the corresponding preferred protein encoded by the HLA allele to which the peptide binds or is predicted to bind, as applicable to the context of this Application.

TABLE 37

| PEPTIDE | ALLELE |
|---|---|
| ANGSLLNY | HLA-A36:01 |
| ANGSLLNYL | HLA-C15:02 |
| | HLA-C08:01 |
| | HLA-C06:02 |
| | HLA-A02:04 |
| | HLA-C12:02 |
| | HLA-B44:02 |
| | HLA-C17:01 |
| | HLA-B38:01 |
| ANGSLLNYLR | HLA-A74:01 |
| | HLA-A31:01 |
| EYMANGSL | HLA-C14:02 |
| | HLA-C14:03 |
| | HLA-A24:02 |
| EYMANGSLL | HLA-C14:02 |
| | HLA-C14:03 |
| | HLA-A33:03 |
| | HLA-C04:01 |
| | HLA-B15:09 |
| | HLA-B38:01 |
| EYMANGSLLN | HLA-A24:02 |
| | HLA-A23:01 |
| EYMANGSLLNY | HLA-A29:02 |
| GSLLNYLR | HLA-A31:01 |
| | HLA-A74:01 |
| GSLLNYLREM | HLA-B58:02 |
| | HLA-B57:01 |
| ITEYMANGS | HLA-A01:01 |
| ITEYMANGSL | HLA-A01:01 |
| ITEYMANGSLL | HLA-A01:01 |
| MANGSLLNY | HLA-C02:02 |
| | HLA-C03:02 |
| | HLA-B53:01 |
| | HLA-C12:02 |
| | HLA-C12:03 |
| | HLA-A36:01 |
| | HLA-A26:01 |
| | HLA-A25:01 |
| | HLA-B57:01 |
| | HLA-A03:01 |
| | HLA-B46:01 |
| | HLA-B15:03 |
| | HLA-A33:03 |
| | HLA-B35:03 |
| | HLA-A11:01 |
| MANGSLLNYL | HLA-C17:01 |
| | HLA-C02:02 |
| | HLA-B35:01 |
| | HLA-C03:03 |
| | HLA-C08:01 |
| | HLA-B35:03 |
| | HLA-C12:02 |
| | HLA-C01:02 |
| | HLA-C03:04 |
| | HLA-C08:02 |
| MANGSLLNYLR | HLA-A33:03 |
| | HLA-A74:01 |
| NGSLLNYL | HLA-B14:02 |
| NGSLLNYLR | HLA-A68:01 |
| | HLA-A33:03 |
| | HLA-A31:01 |
| | HLA-A74:01 |
| SLLNYLREM | HLA-A02:04 |
| | HLA-A02:03 |
| | HLA-C03:02 |
| | HLA-A03:01 |
| | HLA-A32:01 |
| | HLA-A02:07 |
| | HLA-C14:03 |
| | HLA-C14:02 |
| | HLA-A31:01 |
| | HLA-A30:02 |
| | HLA-A74:01 |
| | HLA-C06:02 |
| | HLA-B15:03 |
| | HLA-B46:01 |
| | HLA-B13:02 |
| | HLA-A25:01 |
| | HLA-A29:02 |
| | HLA-C01:02 |
| SLLNYLREMR | HLA-A74:01 |
| | HLA-A31:01 |
| TEYMANGSL | HLA-B14:02 |
| | HLA-B49:01 |
| | HLA-B44:03 |
| | HLA-B44:02 |
| | HLA-B37:01 |
| | HLA-B15:09 |
| | HLA-B41:01 |
| | HLA-B50:01 |
| TEYMANGSLL | HLA-B40:01 |
| | HLA-B44:03 |
| | HLA-B49:01 |
| | HLA-B44:02 |
| | HLA-B40:02 |
| TEYMANGSLLNY | HLA-B44:03 |
| YMANGSLL | HLA-B15:09 |
| | HLA-C03:04 |
| | HLA-C03:03 |
| | HLA-C17:01 |
| | HLA-C03:02 |
| | HLA-C14:03 |
| | HLA-C14:02 |
| | HLA-C04:01 |
| | HLA-C02:02 |
| | HLA-A01:01 |
| YMANGSLLN | HLA-A29:02 |
| | HLA-A01:01 |
| YMANGSLLNY | HLA-A29:02 |
| | HLA-A36:01 |
| | HLA-B46:01 |
| | HLA-A25:01 |
| | HLA-B15:01 |
| | HLA-A26:01 |
| | HLA-A30:02 |
| | HLA-A32:01 |

Exemplary mutations in the EGFR gene, which are prevalent in various types of cancer are presented in Table 40A-40D. The table also provides exemplary EGFR neoantigenic peptides. Mutations involving single amino acid substitutions prevalent in cancer are listed in Tables 40A-40C. Exemplary mutations involving a deletion or deletion and insertion are resented in Table 40D.

TABLE 40A
Exemplary EGFR point mutations in cancer and mutant peptides

| | | Mutation | | | |
|---|---|---|---|---|---|
| | | (amino | Mutation Sequence | | |
| Gene | Nucleotide | acid) | Context | Neopeptides | Cancer |
| EGFR | c.1786C > T | p.P596S | CTGRGPDNCIQCAHYID | CVKTCSAGV, | GBM |
| | | | GPRCVRTC[p.P596S] | VKTCSAGVM, | |
| | | | SAGVMGENNTLVWKYAD | CVKTCSAGVM | |
| | | | AGHVCHLCH | | |
| EGFR | c.1787C > T | p.P596L | CTGRCPDNCIQCAHYID | CVKTCLAGV, | GBM |
| | | | GPHCVKTC[p.S596L] | GPHCVKTCL, | |
| | | | LAGVMGENNTLVWKYAD | VKTCLAGVM | |
| | | | AGHVCHLCH | | |
| EGFR | c.1793G > C | p.G598A | GRGPDNCIQCAHYIDGP | CVKTCPAAV, | GBM |
| | | | HCVKTCPA[p.G598A] | VKTCPAAVM, | |
| | | | AVMGENNTLVWKYADAG | AVMGENNTL, | |
| | | | HVCHLCHPN | AVMGENNTLV, | |
| | | | | CVKTCPAAVM, | |
| | | | | AAVMGENNTL | |
| EGFR | c.1793G > T | p.G598V | GRGPDNCIQCAHYIDGP | CVKCPAVV, | GBM |
| | | | HCVKTCPA[p.G598V] | VKTCPAVVM, | |
| | | | VVMGENNTLVWKYADAG | VVMGENNTLV, | |
| | | | HVCHLCHPN | CVKTCPAVVM | |
| EGFR | c.185T > G | p.162R | KLTQLGTFEDHFLSLQR | MFNNCEVVR, | GBM |
| | | | MFNNCLVV[p.L62R]R | EVVRGNLEI, | |
| | | | GNLEITYVQRNYDLSFL | VRGNLETTY, | |
| | | | KTQEVAG | RMFNNCEVVR, | |
| | | | | VVRGNLEITY, | |
| | | | | CEVVRGNIE | |
| EGFR | c.2125G > A | p.E709K | QERELVEPLTPSGEAPN | RILKKTEFK, | GBM |
| | | | QALLRILK[p.E709K] | ILKKTEFKK, | |
| | | | KTEFKKIKVLGSGAFGT | QALLRILKK, | |
| | | | VYKGLWIP | LRILKTEF, | |
| | | | | RILKKTEFKK, | |
| | | | | NQALLRILKK, | |
| | | | | LLRLKKTEF | |
| EGFR | c.2156G > C | p.G719A | PSGEAPNQALLRILKET | A5GAFGTVY, | LUAD |
| | | | EFKKIKVL[p.G719A] | VLASGAFGT, | |
| | | | ASGAFGTVYKGLWIPEG | LASAFGTY, | |
| | | | EKVKIPVAI | KIKVLASGA, | |
| | | | | KVLASGAFG, | |
| | | | | IKVLASGAF, | |
| | | | | KKIKVLASG, | |
| | | | | VLASG, | |
| | | | | VLASGAFGTV, | |
| | | | | ASGAFGTVYK, | |
| | | | | KIKVLASGAF, | |
| | | | | LASGAFGTVY, | |
| | | | | KKIKVLASGA, | |
| | | | | TEFKKIKVIA | |
| EGFR | c.2235, | p.ELREA746deI | GAFGTVYKGLWIPEGEK | AIKTSPKANK, | LUAD |
| | 2249 > \| | | VKIPVAIK | KVKIPVAIKT, | |
| | GGAATTA | | [p.ELREA745del]TS | KT5PKANKEI | |
| | AGAGAAG | | PKANKEILDEAYVMASV | | |
| | C | | DNPHVCRLLGICLTSTV | | |
| | | | QLIT | | |
| EGFR | c.2303G > T | p.57681 | AIKELREQATSPKANKE | MAIVDNPHV, | LUAD |
| | | | ILDEAYVMA[p.57681] | VMAIVDNPH, | |
| | | | VDNPHVCRLLGICLTST | DEAYVMAIV, | |
| | | | VQLITQLM | LDEAYYMAI, | |
| | | | | RDEAYVMAI, | |
| | | | | VMAIVDNPHV, | |
| | | | | AIVDNPHVCR, | |
| | | | | YVMAIVDNPH, | |
| | | | | DEAYVMAIVD | |
| EGFR | c.2512C > A | p.L838M | YLLNVVCVQLAKGMNYL | RLVHRDMAA, | KIRC |
| | | | EDRRLVHRD[p.L838M] | DMAARNVLV, | |
| | | | MAARNVLVKTPQHVKIT | MAARNVLVK, | |
| | | | DFGLAKLLG | LVHRDMARR, | |
| | | | | RDMAARNVL, | |
| | | | | RLVHRDMAAR, | |
| | | | | DMAARNVLVK, | |
| | | | | HRDMAARNVL, | |
| | | | | RDMAARNVLV | |
| EGFR | c.2573T > G | p.L858R | LVHRDLAARNVLVKTPQ | KITDGRAK, | LUAD |
| | | | HIVKITDEG[p.L858R] | HVKITDFGR, | |
| | | | RAKLGAEEKEYHAFGGR | FGRAKLLGA, | |
| | | | VPIKWMAL | HVKITDFGRA, | |
| | | | | RAKLIGAEEK | |
| EGFR | c.2582T > A | p.L861Q | RDLAARNVLVKTPQHVR | LAKQLGAEEK, | LUSC |
| | | | ITDFGLAK[p.L861Q] | KQLGAEEKEY | |
| | | | QLGAEEKEYHAEGGKVP | | |
| | | | IKWMALES1 | | |
| EGFR | c.323G > A | p.R108K | QEVAGYVLIALNTVERI | QHKGNMYY, | GBM |
| | | | PLENLQAIE[p.R108K] | LQHKGNMY, | |
| | | | KGNMYYENSYALVLSNY | LQHKGNMYY, | |
| | | | DANKTGLK | KGNMYYENSY | |
| EGFR | c.754C > T | p.R252C | SPSDECLMNQCAAGEIG | RESDCLVCC | GBM |
| | | | PNESDLVC[p.R252C] | | |
| | | | CKFRDEATCKDTCPPLM | | |
| | | | LYNPTTYQM | | |
| EGFR | c.865G > A | p.A289T | CPPLMLYNPTTYQMDVN | YSFGTTCVK, | GBM |
| | | | PEGKYSFG[p.A289T] | TTCVKKCPR, | |
| | | | TTCVKKCPRNYVVTDHG | GKYSFGTTC, | |
| | | | SCVRACGAD | YSFGTTCVKK, | |
| | | | | KYSFGTTCVK, | |
| | | | | GTTCVKKCPR, | |
| | | | | GKYSFGTTCV | |
| EGFR | c.866C > A | p.A289D | CPPLMLYNPTTYQMDVN | YSFGDTCVK, | GBM |
| | | | PEGKYSFG[p.A289D] | DTCVKKCPR, | |
| | | | TCVKKCPRNYVVTDHGS | GKYSFGDTC, | |
| | | | CVRACGAD | YSFGDTCVKK, | |
| | | | | KYSFGTCVK, | |
| | | | | GKYSFGDTCV | |
| EGFR | c.866C > T | p.A289V | CPPLMLYNPTTYQMDVN | YSFGVTCVK, | GBM |
| | | | PEGKYSFG[p.A289V] | KYSFGVTCV, | |
| | | | VTCVKKCPRNYVVTDHG | VTCVKKCPR, | |
| | | | SCVRACGAD | GKYSFGVTC, | |
| | | | | YSFGVTCVKK, | |
| | | | | KYSFGVTCVK, | |
| | | | | GVTCVKKCPR, | |
| | | | | GKYSFGVTCV | |
| EGFR | c.910C > T | p.H304Y | VNPEGKYSFGATCVKKI | VVTDYGSCV, | GBM |
| | | | CPRNYVVTD[p.H304Y] | YVVTDYGSCV, | |
| | | | YGSCVRACGADSYEMEE | VVTDYGSCVR, | |
| | | | DGVRKCKKC | CPRNYVVTDY | |

TABLE 40B
Exemplary EGFR point
mutations in cancer and mutant peptides

| Mutation | | | |
|---|---|---|---|
| amino acid | Neo- | | |
| (nucleotide) | peptides | Cancer | Allele |
| EGFRp.L858R | FGRAKLLGA | Lung | HLA.B08.01 |
| (uc003tqk.2) | | adeno- | |
| | | carcinoma | |
| EGFRp.L858R | KITDFGRAK | Lung | HLA.A03.01 |
| (uc003tqk.2) | | adeno- | HLA.A11.01 |
| | | carcinoma | HLA.A30.01 |

TABLE 40C
Exemplary EGFR point
mutations in cancer and mutant peptides

| | Mutation | | |
|---|---|---|---|
| EGFR | Sequence | | |
| Mutation | Context | Neopeptides | Disease |
| EGFR, | GICLTSTV | VQLIMQLMPF, | CRC |
| T790M | QLIMQLMP | STVQLIMQLM, | |
| | FGCLLDY | QLIMQLMPF, | |
| | | MQLMPFGCLL, | |
| | | LIMQLMPF, | |
| | | LTSTVQLIM, | |
| | | STVQLIMQL, | |
| | | TSTVQLIMQL, | |
| | | TVQLIMQL, | |
| | | TVQLIMQLM, | |
| | | VQLIMQLM, | |
| | | CLTSTVQLIM, | |
| | | IMQLMPFGC, | |
| | | IMQLMPFGCL, | |
| | | LIMQLMPFG, | |
| | | LIMQLMPFGC, | |
| | | QLIMQLMPFG | |
| EGFR, | SLNITSLG | IIRNRGENSCK | NSCLC, |
| S492R | LRSLKEIS | | PRAD |
| | DGDVIISG | | |
| | NKNLCYAN | | |
| | TINWKKLF | | |
| | GTSGQKTK | | |
| | IIRNRGEN | | |
| | SCKATGQV | | |
| | CHALCSPE | | |
| | GCWGPEPR | | |
| | DCVSCRNV | | |
| | SRGRECVD | | |
| | KCNLL | | |

TABLE 40D
Exemplary EGFR deletion mutation,
fusion mutations in cancer

| | Mutation | | |
|---|---|---|---|
| EGFR | Sequence | Neo- | |
| Mutation | Context | peptides | Disease |
| EGFRvIII | MRPSGTAGAALLALLAALC | ALEEKKGNYV | GBM |
| (internal | PASRALEEKK:G:NYVVTD | | |
| deletion) | HGSCVRACGADSYEMEEDG | | |
| | VRKCKKCEGPCRKVCNGIG | | |
| | IGEFKD | | |
| EGFR: | LPQPPICTIDVYMIMVKCW | IQLQDKFEHL | GBM, |
| SEPT14 | MIDADSRPKFRELIIEFSK | QLQDKFEHL | Glioma, |
| | MARDPQRYLVIQ::LQDKF | QLQDKFEHLK | Head and |
| | EHLKMIQQEEIRKLEEEKK | YLVIQLQDKF | Neck |
| | QLEGEIIDFYKMKAASEAL | | Cancer |
| | QTQLSTD | | |

the Tables above, for one or more of the exemplary fusions, a sequence that comes before the first “:” belongs to an exon sequence of a polypeptide encoded by a first gene, a sequence that comes after the second “:” belongs to an exon sequence of a polypeptide encoded by a second gene, and an amino acid that appears between “:” symbols is encoded by a codon that is split between the exon sequence of a polypeptide encoded by a first gene and the exon sequence of a polypeptide encoded by a second gene.

However, in some embodiments, for example, NAB:STAT6, the NAB exon is linked to the 5′ UTR of STAT6 and the first amino acid that appears after the Junction is the normal start codon of STAT6 (there is no frame present at this site (as it is not normally translated).

AR-V7 in the tables above can also be considered, in some embodiments, a splice variant of the AR gene that encodes a protein that lacks the ligand binding domain found in full length AR.

In some embodiments, sequencing methods are used to identify tumor specific mutations. Any suitable sequencing method can be used according to the present disclosure, for example, Next Generation Sequencing (NGS) technologies. Third Generation Sequencing methods might substitute for the NGS technology in the future to speed up the sequencing step of the method. For clarification purposes: the terms “Next Generation Sequencing” or “NGS” in the context of the present disclosure mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, for example, within 1-7 days or within less than 24 hours and allow, in principle, single cell sequencing approaches. Multiple NGS platforms which are commercially available or which are mentioned in the literature can be used in the context of the present disclosure e.g. those described in detail in WO 2012/159643.

In certain embodiments, the peptide described herein can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120, about 150, about 200, about 300, about 350, about 400, about 450, about 500, about 600, about 700, about 800, about 900, about 1,000, about 1,500, about 2,000, about 2,500, about 3,000, about 4,000, about 5,000, about 7,500, about 10,000 amino acids or greater amino acid residues, and any range derivable therein. In specific embodiments, a neoantigenic peptide molecule is equal to or less than 100 amino acids.

In some embodiments, the peptides can be from about 8 and about 50 amino acid residues in length, or from about 8 and about 30, from about 8 and about 20, from about 8 and about 18, from about 8 and about 15, or from about 8 and about 12 amino acid residues in length. In some embodiments, the peptides can be from about 8 and about 500 amino acid residues in length, or from about 8 and about 450, from about 8 and about 400, from about 8 and about 350, from about 8 and about 300, from about 8 and about 250, from about 8 and about 200, from about 8 and about 150, from about 8 and about 100, from about 8 and about 50, or from about 8 and about 30 amino acid residues in length.

In some embodiments, the peptides can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues in length. In some embodiments, the peptides can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or more amino acid residues in length. In some embodiments, the peptides can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or less amino acid residues in length. In some embodiments, the peptides can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or less amino acid residues in length.

In some embodiments, the peptides has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, or at least 500 amino acids.

In some embodiments, the peptides has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, or at most 500 amino acids.

A longer peptide can be designed in several ways. In some embodiments, when HLA-binding peptides are predicted or known, a longer peptide comprises (1) individual binding peptides with extensions of 2-5 amino acids toward the N- and C-terminus of each corresponding gene product; or (2) a concatenation of some or all of the binding peptides with extended sequences for each. In other embodiments, when sequencing reveals a long (>10 residues) neoepitope sequence present in the tumor (e.g., due to a frameshift, read-through or intron inclusion that leads to a novel peptide sequence), a longer peptide could consist of the entire stretch of novel tumor-specific amino acids as either a single longer peptide or several overlapping longer peptides. In some embodiments, use of a longer peptide is presumed to allow for endogenous processing by patient cells and can lead to more effective antigen presentation and induction of T cell responses. In some embodiments, two or more peptides can be used, where the peptides overlap and are tiled over the long neoantigenic peptide.

In some embodiments, the peptides can have a pI value of from about 0.5 to about 12, from about 2 to about 10, or from about 4 to about 8. In some embodiments, the peptides can have a pI value of at least 4.5, 5, 5.5, 6, 6.5, 7, 7.5, or more. In some embodiments, the peptides can have a pI value of at most 4.5, 5, 5.5, 6, 6.5, 7, 7.5, or less.

In some embodiments, the peptide described herein can be in solution, lyophilized, or can be in crystal form. In some embodiments, the peptide described herein can be prepared synthetically, by recombinant DNA technology or chemical synthesis, or can be isolated from natural sources such as native tumors or pathogenic organisms. Neoepitopes can be synthesized individually or joined directly or indirectly in the peptide. Although the peptide described herein can be substantially free of other naturally occurring host cell proteins and fragments thereof, in some embodiments, the peptide can be synthetically conjugated to be joined to native fragments or particles.

In some embodiments, the peptide described herein can be prepared in a wide variety of ways. In some embodiments, the peptides can be synthesized in solution or on a solid support according to conventional techniques. Various automatic synthesizers are commercially available and can be used according to known protocols. See, for example, Stewart & Young, Solid Phase Peptide Synthesis, 2d. Ed., Pierce Chemical Co., 1984. Further, individual peptides can be joined using chemical ligation to produce larger peptides that are still within the bounds of the present disclosure.

Alternatively, recombinant DNA technology can be employed wherein a nucleotide sequence which encodes the peptide inserted into an expression vector, transformed or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1989). Thus, recombinant peptides, which comprise one or more neoantigenic peptides described herein, can be used to present the appropriate T cell epitope.

In some embodiments, the peptide is encoded by a gene with a point mutation resulting in an amino acid substitution of the native peptide. In some embodiments, the peptide is encoded by a gene with a point mutation resulting in frame shift mutation. A frameshift occurs when a mutation disrupts the normal phase of a gene's codon periodicity (also known as “reading frame”), resulting in the translation of a non-native protein sequence. It is possible for different mutations in a gene to achieve the same altered reading frame. In some embodiments, the peptide is encoded by a gene with a mutation resulting in fusion polypeptide, in-frame deletion, insertion, expression of endogenous retroviral polypeptides, and tumor-specific overexpression of polypeptides. In some embodiments, the peptide is encoded by a fusion of a first gene with a second gene. In some embodiments, the peptide is encoded by an in-frame fusion of a first gene with a second gene. In some embodiments, the peptide is encoded by a fusion of a first gene with an exon of a splice variant of the first gene. In some embodiments, the peptide is encoded by a fusion of a first gene with a cryptic exon of the first gene. In some embodiments, the peptide is encoded by a fusion of a first gene with a second gene, wherein the peptide comprises an amino acid sequence encoded by an out of frame sequence resulting from the fusion.

In some aspects, the present disclosure provides a composition comprising at least two or more than two peptides. In some embodiments, the composition described herein contains at least two distinct peptides. In some embodiments, the composition described herein contains a first peptide comprising a first neoepitope and a second peptide comprising a second neoepitope. In some embodiments, the first and second peptides are derived from the same protein. The at least two distinct peptides may vary by length, amino acid sequence or both. The peptides can be derived from any protein known to or have been found to contain a tumor specific mutation. In some embodiments, the composition described herein comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, the composition described herein comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof.

In some embodiments, the peptide can be derived from a protein with a substitution mutation, e.g., the KRAS G12C, G12D, G12V, Q61H or Q61L mutation, or the NRAS Q61K or Q61R mutation, or BTK C481S mutation, or EGFR S492R, or the EGFR T490M mutation. The substitution may be positioned anywhere along the length of the peptide. For example, it can be located in the N terminal third of the peptide, the central third of the peptide or the C terminal third of the peptide. In another embodiment, the substituted residue is located 2-5 residues away from the N terminal end or 2-5 residues away from the C terminal end. The peptides can be similarly derived from tumor specific insertion mutations where the peptide comprises one or more, or all of the inserted residues.

In some embodiments, the first peptide comprises at least one an additional mutation. In some embodiments, one or more of the at least one additional mutation is not a mutation in the first neoepitope. In some embodiments, one or more of the at least one additional mutation is a mutation in the first neoepitope. In some embodiments, the second peptide comprises at least one additional mutation. In some embodiments, one or more of the at least one additional mutation is not a mutation in the second neoepitope. In some embodiments, one or more of the at least one additional mutation is a mutation in the second neoepitope.

In some aspects, the present disclosure provides a composition comprising a single polypeptide comprises the first peptide and the second peptide, or a single polynucleotide encodes the first peptide and the second peptide. In some embodiments, the composition provided herein comprises one or more additional peptides, wherein the one or more additional peptides comprise a third neoepitope. In some embodiments, the first peptide and the second peptide are encoded by a sequence transcribed from the same transcription start site. In some embodiments, the first peptide is encoded by a sequence transcribed from a first transcription start site and the second peptide is encoded by a sequence transcribed from a second transcription start site. In some embodiments, wherein the polypeptide has a length of at least 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the polypeptide comprises a first sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence; and a second sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence. In some embodiments, the polypeptide comprises a first sequence of at least 8 or 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence; and a second sequence of at least 16 or 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence.

In some embodiments, the second peptide is longer than the first peptide. In some embodiments, the first peptide is longer than the second peptide. In some embodiments, the first peptide has a length of at least 9; 10; 11; 12; 13; 14; 15; 16; 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the second peptide has a length of at least 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the first peptide comprises a sequence of at least 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a corresponding wild-type sequence. In some embodiments, the second peptide comprises a sequence of at least 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence.

In some embodiments, the first peptide, the second peptide or both comprise at least one flanking sequence, wherein the at least one flanking sequence is upstream or downstream of the neoepitope. In some embodiments, the at least one flanking sequence has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence. In some embodiments, the at least one flanking sequence comprises a non-wild-type sequence. In some embodiments, the at least one flanking sequence is a N-terminus flanking sequence. In some embodiments, the at least one flanking sequence is a C-terminus flanking sequence. In some embodiments, the at least one flanking sequence of the first peptide has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the at least one flanking sequence of the second peptide. In some embodiments, the at least one flanking region of the first peptide is different from the at least one flanking region of the second peptide. In some embodiments, the at least one flanking residue comprises the mutation.

In some embodiments, neoantigenic peptide with the flanking sequences comprises a polypeptide, which can be represented by a formula (N-terminal Xaa)_{N}-(Xaa_{BTK})_{P}-(Xaa-C terminal)c, where (Xaa_{BTK})_{P} is a mutant BTK peptide sequence comprising at least 8 contiguous amino acids of a mutant BTK protein, P is an integer greater than 7; N is (i) 0 or (ii) an integer greater than 2; (N-terminal Xaa)_{N} is any amino acid sequence heterologous to the mutant protein; C is (i) 0 or (ii) an integer greater than 2; (Xaa-C terminal)_{C} is any amino acid sequence heterologous to the mutant BTK protein; and, both N and C are not 0.

In some embodiments, neoantigenic peptide with the flanking sequences comprises a polypeptide, which can be represented by a formula (N-terminal Xaa)_{N}-(Xaa_{EGFR})_{P}-(Xaa-C terminal)c, where (Xaa_{EGFR})_{P} is a mutant EGFR peptide sequence comprising at least 8 contiguous amino acids of a mutant EGFR protein, P is an integer greater than 7; N is (i) 0 or (ii) an integer greater than 2; (N-terminal Xaa)_{N} is any amino acid sequence heterologous to the mutant EGFR protein; C is (i) 0 or (ii) an integer greater than 2; (Xaa-C terminal)_{C} is any amino acid sequence heterologous to the mutant EGFR protein; and, both N and C are not 0.

In some embodiments, a peptide comprises a neoepitope sequence comprising at least one mutant amino acid. In some embodiments, a peptide comprises a neoepitope sequence comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more mutant amino acids. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid. In some embodiments, a peptide comprises a neoepitope sequence derived from a protein comprising at least one mutant amino acid; at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid; and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid.

In some embodiments, a peptide comprises a neoantigenic peptide sequence depicted in Tables 1 or 2. In some embodiments, a peptide comprises a neoepitope sequence depicted in Tables 1 or 2. In some embodiments, a peptide comprises a neoepitope sequence comprising at least one mutant amino acid (underlined amino acid) as depicted in Tables 1 or 2. In some embodiments, a peptide comprises a neoepitope sequence comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more mutant amino acids (underlined amino acids) as depicted in Tables 1 or 2. In some embodiments, a peptide comprises a neoantigenic peptide sequence depicted in Tables 34 or 36. In some embodiments, a peptide comprises a neoepitope BTK sequence depicted in Tables 34 or 36. In some embodiments, a peptide comprises a neoepitope sequence comprising at least one mutant amino acid as depicted in Tables 34 or 36. In some embodiments, a peptide comprises a neoepitope sequence comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more mutant amino In some embodiments, a peptide comprises a neoepitope sequence comprising at least one mutant amino acid (underlined amino acid) and at least one bolded amino acid as depicted in Tables 1 or 2. In some embodiments, a peptide comprises a neoepitope sequence derived from a protein comprising at least one mutant amino acid (underlined amino acid) and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids as depicted in Tables 1 or 2. In some embodiments, a peptide comprises a neoepitope sequence derived from a protein comprising at least one mutant amino acid (underlined amino acid) and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid as depicted in Tables 1 or 2. In some embodiments, a peptide comprises a neoepitope sequence derived from a protein comprising at least one mutant amino acid (underlined amino acid) and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid as depicted in Tables 1 or 2. In some embodiments, a peptide comprises a neoepitope sequence derived from a protein comprising at least one mutant amino acid (underlined amino acid), at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid, and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid as depicted in Tables 1 or 2.

In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid as depicted in Tables 34 or 36. In some embodiments, a peptide comprises a neoepitope sequence derived from a protein comprising at least one mutant amino acid, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid, and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid as depicted in Tables 34 or 36.

In some embodiments, a peptide comprises a neoantigenic peptide sequence depicted in Tables 40A-40D, 32, or 3A-3D. In some embodiments, a peptide comprises a neoepitope EGFR sequence depicted in Tables 40A-40D. In some embodiments, a peptide comprises a neoepitope sequence comprising at least one mutant amino acid as depicted in Tables 40A-40D. In some embodiments, a peptide comprises a neoepitope sequence comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more mutant amino acids (for example, an underlined amino acid in any one of Tables 40A-40D). In some embodiments, an EGFR peptide comprises a neoepitope sequence comprising at least one mutant amino acid depicted in bold letter as depicted in Tables 40D. In some embodiments, a peptide comprises a neoepitope sequence derived from a protein comprising at least one mutant amino acid (underlined amino acid) and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids as depicted in Tables 40A-40D. In some embodiments, a peptide comprises a neoepitope sequence derived from a protein comprising at least one mutant amino acid (for example, underlined amino acid in Table 40C) and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid as depicted in Tables 40A-40D. In some embodiments, a peptide comprises a neoepitope sequence derived from a protein comprising at least one mutant amino acid (underlined amino acid) and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid as depicted in Tables 40A-40D. In some embodiments, a peptide comprises a neoepitope sequence derived from a protein comprising at least one mutant amino acid (underlined amino acid), at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid, and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid as depicted in Tables 40A-40D.

In some embodiments, a peptide comprises a neoepitope sequence derived from a protein comprising at least one mutant amino acid and a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid, a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

In some embodiments, a peptide comprises a neoepitope sequence derived from a protein comprising at least one mutant amino acid and a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid, a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

In some embodiments, a peptide comprises a neoepitope sequence derived from a protein comprising at least one mutant amino acid (underlined amino acid) as depicted in Tables 1 or 2 and a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid (underlined amino acid) as depicted in Tables 1 or 2 and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid (underlined amino acid) as depicted in Tables 1 or 2, a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

In some embodiments, a peptide comprises a neoepitope sequence derived from a protein comprising at least one mutant amino acid (underlined amino acid) as depicted in Tables 1 or 2 and a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid (underlined amino acid) as depicted in Tables 1 or 2 and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid (underlined amino acid) as depicted in Tables 1 or 2, a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

In some embodiments, an BTK peptide comprises a neoepitope sequence derived from a protein comprising at least one mutant amino acid (underlined amino acid) as depicted in Tables 34 or 36 and a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid as depicted in Tables 34 or 36 and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid as depicted in Tables 34 or 36, a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

In some embodiments, a peptide comprises a neoepitope sequence derived from a protein comprising at least one mutant amino acid (underlined amino acid) as depicted in Tables 34 or 36, and a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid as depicted in Table 34 or 36 and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid (underlined amino acid) as depicted in Table 34 or 36, a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

Exemplary neoantigenic peptides corresponding to the C481S mutation are presented in Table 34. The table also provides a list of HLA alleles, the encoded protein products of which can bind to the peptides. In some embodiments, a peptide comprising a C481S mutation is: MIKEGSMSEDEFIEEAKVMMNLSHEKLVQLYGVCTKQRPIFIITEYMANGSLLNYLREMRHRFQTQQ LLEMCKDVCEAMEYLESKQFLHRDLAARNCLVND. In some embodiments, a peptide comprising a BTK mutation comprises a neoepitope sequence of ANGSLLNY. In some embodiments, a peptide comprising a C481S BTK mutation comprises a neoepitope sequence of ANGSLLNYL. In some embodiments, a peptide comprising a C481S BTK mutation comprises a neoepitope sequence of ANGSLLNYLR. In some embodiments, a peptide comprising a C481S BTK mutation comprises a neoepitope sequence of EYMANGSL. In some embodiments, a peptide comprising a C481S BTK mutation comprises a neoepitope sequence of EYMANGSLLN. In some embodiments, a peptide comprising a C481S BTK mutation comprises a neoepitope sequence of EYMANGSLLNY. In some embodiments, a peptide comprising a C481S BTK mutation comprises a neoepitope sequence of GSLLNYLR. In some embodiments, a peptide comprising a C481S BTK mutation comprises a neoepitope sequence of GSLLNYLREM. In some embodiments, a peptide comprising a C481S BTK mutation comprises a neoepitope sequence of ITEYMANGS. In some embodiments, a peptide comprising a C481S BTK mutation comprises a neoepitope sequence of ITEYMANGSL. In some embodiments, a peptide comprising a C481S BTK mutation comprises a neoepitope sequence of ITEYMANGSLL. MANGSLLNYL. In some embodiments, a peptide comprising a C481S BTK mutation comprises a neoepitope sequence of MANGSLLNYLR. In some embodiments, a peptide comprising a C481S BTK mutation comprises a neoepitope sequence of NGSLLNYL. In some embodiments, a peptide comprising a C481S BTK mutation comprises a neoepitope sequence of NGSLLNYL. In some embodiments, a peptide comprising a C481S BTK mutation comprises a neoepitope sequence of SLLNYLREMR. In some embodiments, a peptide comprising a C481S BTK mutation comprises a neoepitope sequence of TEYMANGSLL; TEYMANGSLLNY. In some embodiments, a peptide comprising a C481S BTK mutation comprises a neoepitope sequence of YMANGSLL.

In some embodiments, an EGFR peptide comprises a neoepitope sequence derived from a protein comprising at least one mutant amino acid (underlined amino acid) as depicted in Tables 40A-40D and a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid (underlined amino acid) as depicted in Tables 40A-40D and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid as depicted in Tables 40A-40D, a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

In some embodiments, a peptide comprises a neoepitope sequence derived from a protein comprising at least one mutant amino acid (underlined amino acid) as depicted in Tables 40A-40D and a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid (underlined amino acid) as depicted in Tables 40A-40D and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid (underlined amino acid) as depicted in Tables 40A-40D, a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

In some embodiments, a peptide comprising an EGFR T790M mutation comprises a sequence of GICLTSTVQLIMQLMPFGCLLDY. In some embodiments, a peptide comprising an EGFR T790M mutation comprises a neoepitope sequence of VQLIMQLMPF. In some embodiments, a peptide comprising an EGFR T790M mutation comprises a neoepitope sequence of STVQLIMQLM. In some embodiments, a mutant EGFR peptide comprising an EGFR T790M mutation comprises a neoepitope sequence of QLIMQLMPF. In some embodiments, a peptide comprising an EGFR T790M mutation comprises a neoepitope sequence of MQLMPFGCLL. In some embodiments, a peptide comprising an EGFR T790M mutation comprises a neoepitope sequence of LIMQLMPF. In some embodiments, a peptide comprising an EGFR T790M mutation comprises a neoepitope sequence of LTSTVQLIM. In some embodiments, a peptide comprising an EGFR T790M mutation comprises a neoepitope sequence of STVQLIMQL. In some embodiments, a peptide comprising an EGFR T790M mutation comprises a neoepitope sequence of TSTVQLIMQL. In some embodiments, a peptide comprising an EGFR T790M mutation comprises a neoepitope sequence of TVQLIMQL. In some embodiments, a peptide comprising an EGFR T790M mutation comprises a neoepitope sequence of TVQLIMQLM. In some embodiments, a peptide comprising an EGFR T790M mutation comprises a neoepitope sequence of VQLIMQLM. In some embodiments, a peptide comprising an EGFR T790M mutation comprises a neoepitope sequence of CLTSTVQLIM. In some embodiments, a peptide comprising an EGFR T790M mutation comprises a neoepitope sequence of IMQLMPFGC. In some embodiments, a peptide comprising an EGFR T790M mutation comprises a neoepitope sequence of IMQLMPFGC. In some embodiments, a peptide comprising an EGFR T790M mutation comprises a neoepitope sequence of IMQLMPFGCL. In some embodiments, a peptide comprising an EGFR T790M mutation comprises a neoepitope sequence of LIMQLMPFG. In some embodiments, a peptide comprising an EGFR T790M mutation comprises a neoepitope sequence of LIMQLMPFGC. In some embodiments, a peptide comprising an EGFR T790M mutation comprises a neoepitope sequence of QLIMQLMPFG.

In some embodiments, a peptide comprising an EGFR, S492R mutation comprises a sequence of SLNITSLGLRSLKEISDGDVIISGNKNLCYANTINWKKLFGTSGQKTKIIRNRGENSCKATGQVCHALC SPEGCWGPEPRDCVSCRNVSRGRECVDKCNLL. In some embodiments, a peptide comprising an EGFR S492R mutation comprises a neoepitope sequence of IIRNRGENSCK.

In some embodiments, an EGFR neopeptide is selected from Table 40A-40D.

In some embodiments, a peptide comprising a deletion mutation in EGFR, such as deletion of G in EGFRvIII (internal deletion), MRPSGTAGAALLALLAALCPASRALEEKK:G:NYVVTDHGSCVRACGADSYEMEEDGVRKCKKCEG PCRKVCNGIGIGEFKD, comprises a neoepitope sequence of ALEEKKGNYV.

In some embodiments, a peptide comprising a mutation depicted in the sequence: LPQPPICTIDVYMIMVKCWMIDADSRPKFRELIIEFSKMARDPQRYLVIQ::LQDKFEHLKMIQQEEIR KLEEEKKQLEGEIIDFYKMKAASEALQTQLSTD, comprises a neoepitope sequence of IQLQDKFEHL. In some embodiments, a peptide comprising a mutation depicted in the sequence: LPQPPICTIDVYMIMVKCWMIDADSRPKFRELIIEFSKMARDPQRYLVIQ::LQDKFEHLKMIQQEEIR KLEEEKKQLEGEIIDFYKMKAASEALQTQLSTD, comprises a neoepitope sequence of QLQDKFEHL. In some embodiments, a peptide comprising a mutation depicted in the sequence: LPQPPICTIDVYMIMVKCWMIDADSRPKFRELIIEFSKMARDPQRYLVIQ::LQDKFEHLKMIQQEEIR KLEEEKKQLEGEIIDFYKMKAASEALQTQLSTD, comprises a neoepitope sequence of QLQDKFEHLK. In some embodiments, a peptide comprising a mutation depicted in the sequence: LPQPPICTIDVYMIMVKCWMIDADSRPKFRELIIEFSKMARDPQRYLVIQ::LQDKFEHLKMIQQEEIR KLEEEKKQLEGEIIDFYKMKAASEALQTQLSTD, comprises a neoepitope sequence of

### Peptide Modification

In some embodiments, the present disclosure includes modified peptides. A modification can include a covalent chemical modification that does not alter the primary amino acid sequence of the antigenic peptide itself. Modifications can produce peptides with desired properties, for example, prolonging the in vivo half-life, increasing the stability, reducing the clearance, altering the immunogenicity or allergenicity, enabling the raising of particular antibodies, cellular targeting, antigen uptake, antigen processing, HLA affinity, HLA stability or antigen presentation. In some embodiments, a peptide may comprise one or more sequences that enhance processing and presentation of epitopes by APCs, for example, for generation of an immune response.

In some embodiments, the peptide may be modified to provide desired attributes. For instance, the ability of the peptides to induce CTL activity can be enhanced by linkage to a sequence which contains at least one epitope that is capable of inducing a T helper cell response. In some embodiments, immunogenic peptides/T helper conjugates are linked by a spacer molecule. In some embodiments, a spacer comprises relatively small, neutral molecules, such as amino acids or amino acid mimetics, which are substantially uncharged under physiological conditions. Spacers can be selected from, e.g., Ala, Gly, or other neutral spacers of nonpolar amino acids or neutral polar amino acids. It will be understood that the optionally present spacer need not be comprised of the same residues and thus may be a hetero- or homo-oligomer. The neoantigenic peptide may be linked to the T helper peptide either directly or via a spacer either at the amino or carboxy terminus of the peptide. The amino terminus of either the neoantigenic peptide or the T helper peptide may be acylated. Examples of T helper peptides include tetanus toxoid residues 830-843, influenza residues 307-319, and malaria circumsporozoite residues 382-398 and residues 378-389.

The peptide sequences of the present disclosure may optionally be altered through changes at the DNA level, particularly by mutating the DNA encoding the peptide at preselected bases such that codons are generated that will translate into the desired amino acids.

In some embodiments, the peptide described herein can contain substitutions to modify a physical property (e.g., stability or solubility) of the resulting peptide. For example, the peptides can be modified by the substitution of a cysteine (C) with α-amino butyric acid (“B”). Due to its chemical nature, cysteine has the propensity to form disulfide bridges and sufficiently alter the peptide structurally so as to reduce binding capacity. Substituting α-amino butyric acid for C not only alleviates this problem, but actually improves binding and cross-binding capability in certain instances. Substitution of cysteine with α-amino butyric acid can occur at any residue of a neoantigenic peptide, e.g., at either anchor or non-anchor positions of an epitope or analog within a peptide, or at other positions of a peptide.

The peptide may also be modified by extending or decreasing the compound's amino acid sequence, e.g., by the addition or deletion of amino acids. The peptides or analogs can also be modified by altering the order or composition of certain residues. It will be appreciated by the skilled artisan that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, but may include non-natural amino acids as well, such as D-isomers, β-γ-δ-amino acids, as well as many derivatives of L-α-amino acids.

In some embodiments, the peptide may be modified using a series of peptides with single amino acid substitutions to determine the effect of electrostatic charge, hydrophobicity, etc. on HLA binding. For instance, a series of positively charged (e.g., Lys or Arg) or negatively charged (e.g., Glu) amino acid substitutions may be made along the length of the peptide revealing different patterns of sensitivity towards various HLA molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an HLA molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding. Amino acid substitutions are typically of single residues. Substitutions, deletions, insertions or any combination thereof may be combined to arrive at a final peptide.

In some embodiments, the peptide described herein can comprise amino acid mimetics or unnatural amino acid residues, e.g. D- or L-naphylalanine; D- or L-phenylglycine; D- or L-2-thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoro-methyl)-phenylalanine; D-ρ-fluorophenylalanine; D- or L-ρ-biphenyl-phenylalanine; D- or L-ρ-methoxybiphenylphenylalanine; D- or L-2-indole(allyl)alanines; and, D- or L-alkylalanines, where the alkyl group can be a substituted or unsubstituted methyl, ethyl, propyl, hexyl, butyl, pentyl, isopropyl, iso-butyl, sec-isotyl, iso-pentyl, or a non-acidic amino acid residues. Aromatic rings of a non-natural amino acid include, e.g., thiazolyl, thiophenyl, pyrazolyl, benzimidazolyl, naphthyl, furanyl, pyrrolyl, and pyridyl aromatic rings. Modified peptides that have various amino acid mimetics or unnatural amino acid residues may have increased stability in vivo. Such peptides may also have improved shelf-life or manufacturing properties.

In some embodiments, a peptide described herein can be modified by terminal-NH₂ acylation, e.g., by alkanoyl (C₁-C₂₀) or thioglycolyl acetylation, terminal-carboxyl amidation, e.g., ammonia, methylamine, etc. In some embodiments these modifications can provide sites for linking to a support or other molecule. In some embodiments, the peptide described herein can contain modifications such as but not limited to glycosylation, side chain oxidation, biotinylation, phosphorylation, addition of a surface active material, e.g. a lipid, or can be chemically modified, e.g., acetylation, etc. Moreover, bonds in the peptide can be other than peptide bonds, e.g., covalent bonds, ester or ether bonds, disulfide bonds, hydrogen bonds, ionic bonds, etc.

In some embodiments, a peptide described herein can comprise carriers such as those well known in the art, e.g., thyroglobulin, albumins such as human serum albumin, tetanus toxoid, polyamino acid residues such as poly L-lysine and poly L-glutamic acid, influenza virus proteins, hepatitis B virus core protein, and the like.

The peptides can be further modified to contain additional chemical moieties not normally part of a protein. Those derivatized moieties can improve the solubility, the biological half-life, absorption of the protein, or binding affinity. The moieties can also reduce or eliminate any desirable side effects of the peptides and the like. An overview for those moieties can be found in Remington's Pharmaceutical Sciences, 20th ed., Mack Publishing Co., Easton, Pa. (2000). For example, neoantigenic peptides having the desired activity may be modified as necessary to provide certain desired attributes, e.g. improved pharmacological characteristics, while increasing or at least retaining substantially all of the biological activity of the unmodified peptide to bind the desired HLA molecule and activate the appropriate T cell. For instance, the peptide may be subject to various changes, such as substitutions, either conservative or non-conservative, where such changes might provide for certain advantages in their use, such as improved HLA binding. Such conservative substitutions may encompass replacing an amino acid residue with another amino acid residue that is biologically and/or chemically similar, e.g., one hydrophobic residue for another, or one polar residue for another. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, III., Pierce), 2d Ed. (1984).

In some embodiments, the peptide described herein may be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Changes to the peptide that may include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids.

Glycosylation can affect the physical properties of proteins and can also be important in protein stability, secretion, and subcellular localization. Proper glycosylation can be important for biological activity. In fact, some genes from eukaryotic organisms, when expressed in bacteria (e.g., E. coli) which lack cellular processes for glycosylating proteins, yield proteins that are recovered with little or no activity by virtue of their lack of glycosylation. Addition of glycosylation sites can be accomplished by altering the amino acid sequence. The alteration to the peptide or protein may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. Embodiments of the present disclosure comprise the generation and use of N-glycosylation variants. Removal of carbohydrates may be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases.

Additional suitable components and molecules for conjugation include, for example, molecules for targeting to the lymphatic system, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemagglutinin, influenza virus nucleoprotein; Keyhole Limpet Hemocyanin (KLH); and hepatitis B virus core protein and surface antigen; or any combination of the foregoing.

Another type of modification is to conjugate (e.g., link) one or more additional components or molecules at the N- and/or C-terminus of a polypeptide sequence, such as another protein (e.g., a protein having an amino acid sequence heterologous to the subject protein), or a carrier molecule. Thus, an exemplary polypeptide sequence can be provided as a conjugate with another component or molecule. In some embodiments, fusion of albumin to the peptide or protein of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Furthermore, albumin itself may be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin (see, e.g., WO2011/051489). Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics.

Additional candidate components and molecules for conjugation include those suitable for isolation or purification. Non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes. Purification methods such as cation exchange chromatography may be used to separate conjugates by charge difference, which effectively separates conjugates into their various molecular weights. The content of the fractions obtained by cation exchange chromatography may be identified by molecular weight using conventional methods, for example, mass spectroscopy, SDS-PAGE, or other known methods for separating molecular entities by molecular weight.

In some embodiments, the amino- or carboxyl-terminus of the peptide or protein sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamics properties of the biopharmaceutical as compared to traditional Fc-fusion conjugates.

The present disclosure contemplates the use of other modifications, currently known or developed in the future, of the peptides to improve one or more properties. One such method for prolonging the circulation half-life, increasing the stability, reducing the clearance, or altering the immunogenicity or allergenicity of the peptide of the present disclosure involves modification of the peptide sequences by hesylation, which utilizes hydroxyethyl starch derivatives linked to other molecules in order to modify the molecule's characteristics. Various aspects of hesylation are described in, for example, U.S. Patent Appln. Nos. 2007/0134197 and 2006/0258607.

Peptide stability can be assayed in a number of ways. For instance, peptidases and various biological media, such as human plasma and serum, have been used to test stability. See, e.g., Verhoef, et al., Eur. J. Drug Metab. Pharmacokinetics 11:291 (1986). Half-life of the peptides described herein is conveniently determined using a 25% human serum (v/v) assay. The protocol is as follows: pooled human serum (Type AB, non-heat inactivated) is dilapidated by centrifugation before use. The serum is then diluted to 25% with RPMI-1640 or another suitable tissue culture medium. At predetermined time intervals, a small amount of reaction solution is removed and added to either 6% aqueous trichloroacetic acid (TCA) or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

Issues associated with short plasma half-life or susceptibility to protease degradation may be overcome by various modifications, including conjugating or linking the peptide or protein sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide or protein sequence are generally soluble in water at room temperature, and have the general formula R—(O—CH₂—CH₂)ₙ—O—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. The present disclosure also contemplates compositions of conjugates wherein the PEGs have different n values and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. For example, cation exchange chromatography may be used to separate conjugates, and a fraction is then identified which contains the conjugate having, for example, the desired number of PEGs attached, purified free from unmodified protein sequences and from conjugates having other numbers of PEGs attached.

PEG may be bound to the peptide or protein of the present disclosure via a terminal reactive group (a “spacer”). The spacer is, for example, a terminal reactive group which mediates a bond between the free amino or carboxyl groups of one or more of the polypeptide sequences and PEG. The PEG having the spacer which may be bound to the free amino group includes N-hydroxysuccinylimide PEG which may be prepared by activating succinic acid ester of PEG with N-hydroxysuccinylimide. Another activated PEG which may be bound to a free amino group is 2,4-bis(O-methoxypolyethyleneglycol)-6-chloro-s-triazine which may be prepared by reacting PEG monomethyl ether with cyanuric chloride. The activated PEG which is bound to the free carboxyl group includes polyoxyethylenediamine.

Conjugation of one or more of the peptide or protein sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to peptide/protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C.

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

### Neoepitopes

A neoepitope comprises a neoantigenic determinant part of a neoantigenic peptide or neoantigenic polypeptide that is recognized by immune system. A neoepitope refers to an epitope that is not present in a reference, such as a non-diseased cell, e.g., a non-cancerous cell or a germline cell, but is found in a diseased cell, e.g., a cancer cell. This includes situations where a corresponding epitope is found in a normal non-diseased cell or a germline cell but, due to one or more mutations in a diseased cell, e.g., a cancer cell, the sequence of the epitope is changed so as to result in the neoepitope. The term “neoepitope” is used interchangeably with “tumor specific neoepitope” in the present specification to designate a series of residues, typically L-amino acids, connected one to the other, typically by peptide bonds between the α-amino and carboxyl groups of adjacent amino acids. The neoepitope can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. The present disclosure provides isolated neoepitopes that comprise a tumor specific mutation from Table 1 or 2. The present disclosure also provided exemplary isolated neoepitopes that comprise a tumor specific mutation from Table 34. This disclosure also provides Exemplary isolated neoepitopes that comprise a tumor specific mutation from Tables 40A-40D and Table 3A-3D.

In some embodiments, neoepitopes described herein for HLA Class I are 13 residues or less in length and usually consist of between about 8 and about 12 residues, particularly 9 or 10 residues. In some embodiments, neoepitopes described herein for HLA Class II are 25 residues or less in length and usually consist of between about 16 and about 25 residues.

In some embodiments, the composition described herein comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, the composition described herein comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof.

In some embodiments, the first neoepitope binds to a class I HLA protein to form a class I HLA-peptide complex. In some embodiments, the second neoepitope binds to a class II HLA a protein to form a class II HLA-peptide complex. In some embodiments, the second neoepitope binds to a class I HLA protein to form a class I HLA-peptide complex. In some embodiments, the first neoepitope binds to a class II HLA protein to form a class II HLA-peptide complex. In some embodiments, the first neoepitope activates CD8⁺ T cells. In some embodiments, the first neoepitope activates CD4⁺ T cells. In some embodiments, the second neoepitope activates CD4⁺ T cells. In some embodiments, the second neoepitope activates CD8⁺ T cells. In some embodiments, a TCR of a CD4⁺ T cell binds to a class II HLA-peptide complex. In some embodiments, a TCR of a CD8⁺ T cell binds to a class II HLA-peptide complex. In some embodiments, a TCR of a CD8⁺ T cell binds to a class I HLA-peptide complex. In some embodiments, a TCR of a CD4⁺ T cell binds to a class I HLA-peptide complex. In some embodiments, a composition comprising neoantigenic C481S BTK peptides comprises a first BTK neoepitope and a second BTK neoepitope. In some embodiments, the first BTK neoepitope comprises a neoepitope selected from Table 34. In some embodiments, the second BTK neoepitope comprises a neoepitope selected from Table 34.

In some embodiments, the first mutant BTK peptide sequence that is selected from Table 34 binds to or is predicted to bind to a protein encoded by an HLA allele listed in Table 34, corresponding to the respective peptide (left column versus right).

In some embodiments, a composition comprising neoantigenic EGFR peptides comprises a first EGFR neoepitope and a second EGFR neoepitope. In some embodiments, the first EGFR neoepitope comprises a neoepitope selected from Table 40A-40D. In some embodiments, the second EGFR neoepitope comprises a neoepitope selected from Table 40A-40D.

In some embodiments, a first mutant EGFR neoepitope is selected from a group consisting of STVQLIMQL, LIMQLMPF, LTSTVQLIM, TVQLIMQL, TSTVQLIMQL, TVQLIMQLM and VQLIMQLM.

In some embodiments, the first mutant EGFR peptide sequence that is selected from a group consisting of STVQLIMQL, LIMQLMPF, LTSTVQLIM, TVQLIMQL, TSTVQLIMQL, TVQLIMQLM and VQLIMQLM, binds to or is predicted to bind to a protein encoded by an HLA-A68:01 allele, an HLA-B15:02 allele, an HLA-A25:01 allele, an HLA-B57:03 allele, an HLA-C12:02 allele, an HLA-C03:02 allele, and HLA-A26:01 allele, an HLA-C12:03 allele, an HLA-C06:02 allele, an HLA-C03:03, an HLA-B52:01 allele, HLA-A30:01 allele, an HLA-C02:02 allele, an HLA-C12:03 allele, an HLA-A11:01 allele, an HLA-A32:01 allele, an HLA-A02:04 allele, an HLA-B15:09 allele, HLA-C17:01 allele, an HLA-C03:04 allele, an HLA-B08:01 allele, an HLA-A01:01 allele, an HLA-B42:01 allele, an HLA-B57:01 allele, an HLA-B14:02 allele, an HLA-B37:01 allele, an HLA-B36:01 allele, an HLA-B38:01 allele, an HLA-C03:03 allele, an HLA-B14:02 allele, an HLA-B37:01 allele, an HLA-A02:03 allele, an HLA-B58:02 allele, an HLA-C08:01 allele, an HLA-B35:01 allele, an HLA-B40:01 allele, and/or an HLA-B35:03 allele.

Table 41 provides a list of exemplary HLA alleles encoding an HLA protein that can bind or is predicted to bind to an EGFR neoantigenic peptide.

HLA-A23:01
HLA-A25:01
HLA-A26:01
HLA-A32:01
HLA-B15:01
HLA-B15:02
HLA-B38:01
HLA-B39:01
HLA-B39:06
HLA-B40:02
HLA-C03:02
HLA-C12:03
HLA-A01:01
HLA-C15:02
HLA-B57:01
HLA-B57:03
HLA-A36:01
HLA-C12:02
HLA-C03:03
HLA-B58:02
HLA-B15:01
HLA-A26:01
HLA-A68:02
HLA-C15:02
HLA-A25:01
HLA-B57:03
HLA-C12:02
HLA-A26:01
HLA-C12:03
HLA-C06:02
HLA-C03:03
HLA-A30:01
HLA-C02:02
HLA-A11:01
HLA-A32:01
HLA-A02:04
HLA-A68:01
HLA-B15:09
HLA-C03:04
HLA-B38:01
HLA-B57:01
HLA-A02:03
HLA-C08:01
HLA-B35:01
HLA-B40:01
HLA-A26:01
HLA-B57:01
HLA-C15:02
HLA-C17:01
HLA-B08:01
HLA-B42:01
HLA-B14:02
HLA-B37:01
HLA-B15:09
HLA-B35:03
HLA-B52:01
HLA-B14:02
HLA-B37:01

Tables 42Ai, 42Aii and 42B show EGFR neoepitopes with predicted HLA subtype specificity.
Tables 5Ai, 5Aii and 5B show EGFR neoepitopes with predicted HLA subtype specificity.

TABLE 42Ai

| | Mutation | | |
|---|---|---|---|
| EGFR | Sequence | | HLA |
| mutation | Context | Peptides | allele |
| S492R | SLNITSLGLRSLKEISDGDVI | IIRNRGENSCK | A03.01 |
| | ISGNKNLCYANTINWKKLFGT | | |
| | SGQKTKIIRNRGENSCKATGQ | | |
| | VCHALCSPEGCWGPEPRDCVS | | |
| | CRNVSRGRECVDKCNLL | | |

TABLE 42Aii

| | Mutation | | |
|---|---|---|---|
| EGFR | Sequence | | HLA |
| mutation | Context | Peptides | allele |
| T790M | IPVAIKELREATSP | CLTSTVQLIM | A01.01, A02.01 |
| | KANKEILDEAYVM | IMQLMPFGC | A02.01 |
| | ASVDNPHVCRLLG | IMQLMPFGCL | A02.01, A24.02, |
| | ICLTSTVQLIMQLM | | B08.01 |
| | PFGCLLDYVREHK | LIMQLMPFG | A02.01 |
| | DNIGSQYLLNWCV | LIMQLMPFGC | A02.01 |
| | QIAKGMNYLEDRR | LTSTVQLIM | A01.01 |
| | LVHRDLAA | MQLMPFGCL | A02.01, B07.02, |
| | | | B08.01 |
| | | MQLMPFGCLL | A02.01, A24.02, |
| | | | B08.01 |
| | | VQLIMQLMPF | A02.01, A24.02, |
| | | | B08.01 |
| | | LIMQLMPF | HLA-C03:02 |
| | | LTSTVQLIM | HLA-C12:03, |
| | | | HLA-A01:01, |
| | | | HLA-C15:02, |
| | | | HLA-B57:01 |
| | | | HLA-B57:03, |
| | | | HLA-A36:01, |
| | | | HLA-C12:02, |
| | | | HLA-C03:03, |
| | | | HLA-B58:02, |
| | | QLIMQLMPF | HLA-A26:01 |
| | | STVQLIMQL | HLA-A68:02, |
| | | | HLA-C15:02, |
| | | | HLA-A25:01, |
| | | | HLA-B57:03, |
| | | | HLA-C12:02, |
| | | | HLA-A26:01, |
| | | | HLA-C12:03, |
| | | | HLA-C06:02, |
| | | | HLA-C03:03, |
| | | | HLA-A30:01, |
| | | | HLA-C02:02, |
| | | | HLA-A11:01, |
| | | | HLA-A32:01, |
| | | | HLA-A02:04, |
| | | | HLA-A68:01, |
| | | | HLA-B15:09, |
| | | | HLA-C03:04, |
| | | | HLA-B38:01, |
| | | | HLA-B57:01, |
| | | | HLA-A02:03, |
| | | | HLA-C08:01, |
| | | | HLA-B35:01, |
| | | | HLA-B40:01 |
| | | STVQLIMQLM | HLA-B57:01 |
| | | TSTVQLIMQL | HLA-C15:02 |
| | | TVQLIMQL | HLA-C17:01, |
| | | | HLA-B08:01, |
| | | | HLA-B42:01, |
| | | | HLA-B14:02, |
| | | | HLA-B37:01, |
| | | | HLA-B15:09 |
| | | TVQLIMQLM | HLA-B35:03 |
| | | VQLIMQLM | HLA-B52:01, |
| | | | HLA-B14:02, |
| | | | HLA-B37:01 |

TABLE 42B

| | Mutation | | |
|---|---|---|---|
| EGFR | Sequence | | HLA |
| mutation | Context | Peptides | allele |
| EGFRvIII | MRPSGTAGAALLALLA | ALEEKKGNYV | A02.01 |
| (internal | ALCPASRALEEKK:G: | | |
| deletion) | NYVVTDHGSCVRACGA | | |
| | DSYEMEEDGVRKCKKC | | |
| | EGPCRKVCNGIGIGEF | | |
| | KD | | |
| EGFR: | LPQPPICTIDVYMIMV | IQLQDKFEHL | A02.01, |
| SEPT14 | KCWMIDADSRPKFREL | | B08.01 |
| | IIEFSKMARDPQRYLV | QLQDKFEHL | A02.01, |
| | IQ::LQDKFEHLKMIQ | | B08.01 |
| | QEEIRKLEEEKKQLEG | QLQDKFEHLK | A03.01 |
| | EIIDFYKMKAASEALQ | YLVIQLQDKF | A02.01, |
| | TQLSTD | | A24.02 |

In some embodiments, the first and the second neoepitopes are different epitopes. In some embodiments, the second neoepitope is longer than the first neoepitope. In some embodiments, the first neoepitope has a length of at least 8 amino acids. In some embodiments, the first neoepitope has a length of from 8 to 12 amino acids. In some embodiments, the first neoepitope comprises a sequence of at least 8 contiguous amino acids, wherein at least 1 of the 8 contiguous amino acids are different at corresponding positions of a wild-type sequence. In some embodiments, the first neoepitope comprises a sequence of at least 8 contiguous amino acids, wherein at least 2 of the 8 contiguous amino acids are different at corresponding positions of a wild-type sequence. In some embodiments, the second neoepitope has a length of at least 16 amino acids. In some embodiments, the second neoepitope has a length of from 16 to 25 amino acids. In some embodiments, the second neoepitope comprises a sequence of at least 16 contiguous amino acids, wherein at least 1 of the 16 contiguous amino acids are different at corresponding positions of a wild-type sequence. In some embodiments, the second neoepitope comprises a sequence of at least 16 contiguous amino acids, wherein at least 2 of the 16 contiguous amino acids are different at corresponding positions of a wild-type sequence.

In some embodiments, the neoepitope comprises at least one anchor residue. In some embodiments, the first neoepitope, the second neoepitope or both comprises at least one anchor residue. In one embodiment, the at least one anchor residue of the first neoepitope is at a canonical anchor position or a non-canonical anchor position. In another embodiment, the at least one anchor residue of the second neoepitope is at a canonical anchor position or a non-canonical anchor position. In yet another embodiment, the at least one anchor residue of the first neoepitope is different from the at least one anchor residue of the second neoepitope.

In some embodiments, the at least one anchor residue is a wild-type residue. In some embodiments, the at least one anchor residue is a substitution. In some embodiments, at least one anchor residue does not comprise the mutation.

In some embodiments, the first or the second neoepitope or both comprise at least one anchor residue flanking region. In some embodiments, the neoepitope comprises at least one anchor residue. In some embodiments, the at least one anchor residues comprises at least two anchor residues. In some embodiments, the at least two anchor residues are separated by a separation region comprising at least 1 amino acid. In some embodiments, the at least one anchor residue flanking region is not within the separation region. In some embodiments, the at least one anchor residue flanking region is (a) upstream of a N-terminal anchor residue of the at least two anchor residues; (b) downstream of a C-terminal anchor residue of the at least two anchor residues; or both (a) and (b). In some embodiments, the second neopeptide is selected from Table 34.

In some embodiments, the second neoepitope comprises a mutation T790M. In some embodiments, the second neoepitope comprising an EGFR T790M mutation comprises a sequence of VQLIMQLMPF. In some embodiments the second neoepitope comprising an EGFR T790M mutation comprises a sequence of STVQLIMQLM. In some embodiments, the second neoepitope comprising a EGFR T790M mutation comprises a sequence of QLIMQLMPF. In some embodiments, the second neoepitope comprising an EGFR T790M mutation comprises a sequence of MQLMPFGCLL. In some embodiments, the second neoepitope comprising an EGFR T790M mutation comprises a sequence of LIMQLMPF. In some embodiments, the second neoepitope comprising an EGFR T790M mutation comprises a neoepitope sequence of LTSTVQLIM. In some embodiments, the second neopeptide comprising an EGFR T790M mutation comprises a sequence of STVQLIMQL. In some embodiments, the second neoepitope comprising an EGFR T790M mutation comprises a sequence of TSTVQLIMQL. In some embodiments the second neoepitope comprising an EGFR T790M mutation comprises a sequence of TVQLIMQL. In some embodiments the second neoepitope comprising an EGFR T790M mutation comprises a sequence of TVQLIMQLM. In some embodiments the second neoepitope comprising an EGFR T790M mutation comprises a sequence of VQLIMQLM. In some embodiments, the second neoepitope comprising an EGFR T790M mutation comprises a sequence of CLTSTVQLIM. In some embodiments, the second neoepitope comprising an EGFR T790M mutation comprises a sequence of IMQLMPFGC. In some embodiments, the second neoepitope comprising an EGFR T790M mutation comprises a sequence of IMQLMPFGC. In some embodiments, the second neoepitope comprising an EGFR T790M mutation comprises a sequence of IMQLMPFGCL. In some embodiments the second neoepitope comprising an EGFR T790M mutation comprises a neoepitope sequence of LIMQLMPFG. In some embodiments the second neoepitope comprising an EGFR T790M mutation comprises a sequence of LIMQLMPFGC. In some embodiments, the second neoepitope comprising an EGFR T790M mutation comprises a sequence of QLIMQLMPFG.

In some embodiments, the second neoepitope comprising an EGFR S492R mutation. In some embodiments, a peptide comprising an EGFR S492R mutation comprises a neoepitope sequence of IIRNRGENSCK.

In some embodiments, the second EGFR neoepitope comprising a deletion mutation in EGFR, such as deletion of G in EGFRvIII (internal deletion), wherein the neoepitope sequence is ALEEKKGNYV.

In some embodiments, a second neoepitope comprising a mutation depicted in the sequence: LPQPPICTIDVYMIMVKCWMIDADSRPKFRELIIEFSKMARDPQRYLVIQ::LQDKFEHLKMIQQEEIR KLEEEKKQLEGEIIDFYKMKAASEALQTQLSTD, wherein the neoepitope sequence is IQLQDKFEHL. In some embodiments, the second neoepitope sequence is QLQDKFEHL. In some embodiments, the second neoepitope sequence is QLQDKFEHLK. In some embodiments, the second neoepitope sequence is YLVIQLQDKF.

In some embodiments, the second neopeptide is selected from Table 35 or Table 3A-Table 3D.

In some embodiments, the neoepitopes bind an HLA protein (e.g., HLA class I or HLA class II). In some embodiments, the neoepitopes bind an HLA protein with greater affinity than the corresponding wild-type peptide. In some embodiments, the neoepitope has an IC₅₀ of less than 5,000 nM, less than 1,000 nM, less than 500 nM, less than 100 nM, less than 50 nM, or less.

In some embodiments, the neoepitope can have an HLA binding affinity of between about 1 μM and about 1 mM, about 100 μM and about 500 μM, about 500 μM and about 10 μM, about 1 nM and about 1 μM, or about 10 nM and about 1 μM. In some embodiments, the neoepitope can have an HLA binding affinity of at least 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900, or 1,000 nM, or more. In some embodiments, the neoepitope can have an HLA binding affinity of at most 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900, or 1,000 nM.

In some embodiments, the first and/or second neoepitope binds to an HLA protein with a greater affinity than a corresponding wild-type neoepitope. In some embodiments, the first and/or second neoepitope binds to an HLA protein with a K_{D} or an IC₅₀ less than 1,000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class I protein with a K_{D} or an IC₅₀ less than 1,000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class II protein with a K_{D} or an IC₅₀ less than 2,000 nM, 1,500 nM, 1,000 nM, 900 nM, 800 nM, 700 nM, 600 nM, 500 nM, 250 nM, 150 nM, 100 nM, 50 nM, 25 nM or 10 nM.

In an aspect, the first and/or second neoepitope binds to a protein encoded by an HLA allele expressed by a subject. In another aspect, the mutation is not present in non-cancer cells of a subject. In yet another aspect, the first and/or second neoepitope is encoded by a gene or an expressed gene of a subject's cancer cells.

In some embodiments, the first neoepitope comprises a mutation as depicted in column 2 of Table 1 or 2. In some embodiments, the second neoepitope comprises a mutation as depicted in column 2 of Table 1 or 2. In some embodiments, certain antigenic peptides are paired with specific alleles.

A substitution may be positioned anywhere along the length of the neoepitope. For example, it can be located in the N terminal third of the peptide, the central third of the peptide or the C terminal third of the peptide. In another embodiment, the substituted residue is located 2-5 residues away from the N terminal end or 2-5 residues away from the C terminal end. The peptides can be similarly derived from tumor specific insertion mutations where the peptide comprises one or more, or all of the inserted residues.

In some embodiments, the peptide as described herein can be readily synthesized chemically utilizing reagents that are free of contaminating bacterial or animal substances (Merrifield R B: Solid phase peptide synthesis. I. The synthesis of a tetrapeptide. J. Am. Chem. Soc. 85:2149-54, 1963). In some embodiments, peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

### Polynucleotides

Alternatively, a nucleic acid (e.g., a polynucleotide) encoding the peptide of the present disclosure may be used to produce the neoantigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In some embodiments in vitro translation is used to produce the peptide.

Provided herein are neoantigenic polynucleotides encoding each of the neoantigenic peptides described in the present disclosure. The term “polynucleotide”, “nucleotides” or “nucleic acid” is used interchangeably with “mutant polynucleotide”, “mutant nucleotide”, “mutant nucleic acid”, “neoantigenic polynucleotide”, “neoantigenic nucleotide” or “neoantigenic mutant nucleic acid” in the present disclosure. Various nucleic acid sequences can encode the same peptide due to the redundancy of the genetic code. Each of these nucleic acids falls within the scope of the present disclosure. Nucleic acids encoding peptides can be DNA or RNA, for example, mRNA, or a combination of DNA and RNA. In some embodiments, a nucleic acid sequence encoding a peptide is a self-amplifying mRNA (Brito et al., Adv. Genet. 2015; 89:179-233). Any suitable polynucleotide that encodes a peptide described herein falls within the scope of the present disclosure.

The term “RNA” includes and in some embodiments relates to “mRNA.” The term “mRNA” means “messenger-RNA” and relates to a “transcript” which is generated by using a DNA template and encodes a peptide or polypeptide. Typically, an mRNA comprises a 5′-UTR, a protein coding region, and a 3′-UTR. mRNA only possesses limited half-life in cells and in vitro. In some embodiments, the mRNA is self-amplifying mRNA. In the context of the present disclosure, mRNA may be generated by in vitro transcription from a DNA template. The in vitro transcription methodology is known to the skilled person. For example, there is a variety of in vitro transcription kits commercially available.

The stability and translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448, incorporated herein by reference. In order to increase expression of the RNA used according to the present disclosure, it may be modified within the coding region, i.e., the sequence encoding the expressed peptide or protein, without altering the sequence of the expressed peptide or protein, so as to increase the GC-content to increase mRNA stability and to perform a codon optimization and, thus, enhance translation in cells.

The term “modification” in the context of the RNA used in the present disclosure includes any modification of an RNA which is not naturally present in said RNA. In some embodiments, the RNA does not have uncapped 5′-triphosphates. Removal of such uncapped 5′-triphosphates can be achieved by treating RNA with a phosphatase. In other embodiments, the RNA may have modified ribonucleotides in order to increase its stability and/or decrease cytotoxicity. In some embodiments, 5-methylcytidine can be substituted partially or completely in the RNA, for example, for cytidine. Alternatively, pseudouridine is substituted partially or completely, for example, for uridine.

In some embodiments, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In some embodiments, this guanosine is methylated at the 7-position. The term “conventional 5′-cap” refers to a naturally occurring RNA 5′-cap, to the 7-methylguanosine cap (m G). In the context of the present disclosure, the term “5′-cap” includes a 5′-cap analog that resembles the RNA cap structure and is modified to possess the ability to stabilize RNA and/or enhance translation of RNA if attached thereto, in vivo and/or in a cell.

In certain embodiments, an mRNA encoding a neoantigenic peptide of the present disclosure is administered to a subject in need thereof. In some embodiments, the present disclosure provides RNA, oligoribonucleotide, and polyribonucleotide molecules comprising a modified nucleoside, gene therapy vectors comprising same, gene therapy methods and gene transcription silencing methods comprising same. In some embodiments, the mRNA to be administered comprises at least one modified nucleoside.

The polynucleotides encoding peptides described herein can be synthesized by chemical techniques, for example, the phosphotriester method of Matteucci, et al., J. Am. Chem. Soc. 103:3185 (1981). Polynucleotides encoding peptides comprising or consisting of an analog can be made simply by substituting the appropriate and desired nucleic acid base(s) for those that encode the native epitope.

Polynucleotides described herein can comprise one or more synthetic or naturally-occurring introns in the transcribed region. The inclusion of mRNA stabilization sequences and sequences for replication in mammalian cells can also be considered for increasing polynucleotide expression. In addition, a polynucleotide described herein can comprise immunostimulatory sequences (ISSs or CpGs). These sequences can be included in the vector, outside the polynucleotide coding sequence to enhance immunogenicity.

In some embodiments, the polynucleotides may comprise the coding sequence for the peptide or protein fused in the same reading frame to a polynucleotide which aids, for example, in expression and/or secretion of the peptide or protein from a host cell (e.g., a leader sequence which functions as a secretory sequence for controlling transport of a polypeptide from the cell). The polypeptide having a leader sequence is a pre-protein and can have the leader sequence cleaved by the host cell to form the mature form of the polypeptide.

In some embodiments, the polynucleotides can comprise the coding sequence for the peptide or protein fused in the same reading frame to a marker sequence that allows, for example, for purification of the encoded peptide, which may then be incorporated into a personalized disease vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In some embodiments, the polynucleotides may comprise the coding sequence for one or more the presently described peptides or proteins fused in the same reading frame to create a single concatamerized neoantigenic peptide construct capable of producing multiple neoantigenic peptides.

In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585. In another embodiment, a DNA sequence encoding the peptide or protein of interest would be constructed by chemical synthesis using an oligonucleotide synthesizer. Such oligonucleotides can be designed based on the amino acid sequence of the desired peptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Thus, the present disclosure is also directed to vectors, and expression vectors useful for the production and administration of the neoantigenic peptides and neoepitopes described herein, and to host cells comprising such vectors.

### Vectors

In some embodiments, an expression vector capable of expressing the peptide or protein as described herein can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

A large number of vectors and host systems suitable for producing and administering a neoantigenic peptide described herein are known to those of skill in the art, and are commercially available. The following vectors are provided by way of example. Bacterial: pQE70, pQE60, pQE-9 (Qiagen), pBS, pD10, phagescript, psiX174, pBluescript SK, pbsks, pNH8A, pNH16a, pNH18A, pNH46A (Stratagene); ptrc99a, pKK223-3, pKK233-3, pDR540, pRIT5 (Pharmacia); pCR (Invitrogen). Eukaryotic: pWLNEO, pSV2CAT, pOG44, pXT1, pSG (Stratagene) pSVK3, pBPV, pMSG, pSVL (Pharmacia); p75.6 (Valentis); pCEP (Invitrogen); pCEI (Epimmune). However, any other plasmid or vector can be used as long as it is replicable and viable in the host.

For expression of the neoantigenic peptides described herein, the coding sequence will be provided operably linked start and stop codons, promoter and terminator regions, and in some embodiments, and a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts.

Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. Such promoters can also be derived from viral sources, such as, e.g., human cytomegalovirus (CMV-IE promoter) or herpes simplex virus type-1 (HSV TK promoter). Nucleic acid sequences derived from the SV40 splice, and polyadenylation sites can be used to provide the required nontranscribed genetic elements.

Recombinant expression vectors may be used to amplify and express DNA encoding the peptide or protein as described herein. Recombinant expression vectors are replicable DNA constructs which have synthetic or cDNA-derived DNA fragments encoding a peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), acid phosphatase, or heat shock proteins, among others. The heterologous structural sequence is assembled in appropriate phase with translation initiation and termination sequences, and in some embodiments, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Polynucleotides encoding neoantigenic peptides described herein can also comprise a ubiquitination signal sequence, and/or a targeting sequence such as an endoplasmic reticulum (ER) signal sequence to facilitate movement of the resulting peptide into the endoplasmic reticulum.

In some embodiments, the neoantigenic peptide described herein can also be administered and/or expressed by viral or bacterial vectors. Examples of expression vectors include attenuated viral hosts, such as vaccinia or fowlpox. As an example of this approach, vaccinia virus is used as a vector to express nucleotide sequences that encode the neoantigenic peptides described herein. Vaccinia vectors and methods useful in immunization protocols are described in, e.g., U.S. Pat. No. 4,722,848. Another vector is BCG (Bacille Calmette Guerin). BCG vectors are described by Stover et al., Nature 351:456-460 (1991).

A wide variety of other vectors useful for therapeutic administration or immunization of the neoantigenic polypeptides described herein, e.g. adeno and adeno-associated virus vectors, retroviral vectors, Salmonella Typhimurium vectors, detoxified anthrax toxin vectors, Sendai virus vectors, poxvirus vectors, canarypox vectors, and fowlpox vectors, and the like, will be apparent to those skilled in the art from the description herein. In some embodiments, the vector is Modified Vaccinia Ankara (VA) (e.g. Bavarian Noridic (MVA-BN)).

Among vectors that may be used in the practice of the present disclosure, integration in the host genome of a cell is possible with retrovirus gene transfer methods, often resulting in long term expression of the inserted transgene. In some embodiments, the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the present disclosure). Moreover, lentiviral vectors are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the present disclosure include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the present disclosure is a minimal non-primate lentiviral vector, such as a lentiviral vector based on the equine infectious anemia virus (EIAV). The vectors may have cytomegalovirus (CMV) promoter driving expression of the target gene. Accordingly, the present disclosure contemplates amongst vector(s) useful in the practice of the present disclosure: viral vectors, including retroviral vectors and lentiviral vectors.

Also useful in the practice of the present disclosure is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the present disclosure, mention is made of U.S. Pat. No. 6,955,808. The adenovirus vector used can be selected from the group consisting of the Ad5, Ad35, Ad11, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Ad11 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4; E1 and E3; and E1 and E4 can be used in accordance with the present disclosure.

Furthermore, “gutless” adenovirus vectors, in which all viral genes are deleted, can also be used in accordance with the present disclosure. Such vectors require a helper virus for their replication and require a special human 293 cell line expressing both Ela and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present disclosure, and can even be used for co-delivery of a large number of heterologous inserts/genes.

In some embodiments, the delivery is via an adenovirus, which may be at a single booster dose. In some embodiments, the adenovirus is delivered via multiple doses. In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element.

For ubiquitous expression, the following promoters can be used: CMV, CAG, CBh, PGK, SV40, Ferritin heavy or light chains, etc. For brain expression, the following promoters can be used: Synapsin I for all neurons, CaMK II alpha for excitatory neurons, GAD67 or GAD65 or VGAT for GABAergic neurons, etc. Promoters used to drive RNA synthesis can include: Pol III promoters such as U6 or H1. The use of a Pol II promoter and intronic cassettes can be used to express guide RNA (gRNA). With regard to AAV vectors useful in the practice of the present disclosure, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein. As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. In some embodiments the delivery is via an AAV. The dosage may be adjusted to balance the therapeutic benefit against any side effects.

In some embodiments, a Poxvirus is used in the presently described composition. These include orthopoxvirus, avipox, vaccinia, MVA, NYVAC, canarypox, ALVAC, fowlpox, TROVAC, etc. (see e.g., Verardi et al., Hum. Vaccin. Immunother. 2012 July; 8(7):961-70; and Moss, Vaccine. 2013; 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels. Information concerning poxviruses that may be used in the practice of the present disclosure, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC.1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature.

In some embodiments, the vaccinia virus is used in the disease vaccine or immunogenic composition to express an antigen. (Rolph et al., Recombinant viruses as vaccines and immunological tools. Curr. Opin. Immunol. 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In some embodiments, ALVAC is used as a vector in a disease vaccine or immunogenic composition. ALVAC is a canarypox virus that can be modified to express foreign transgenes and has been used as a method for vaccination against both prokaryotic and eukaryotic antigens (Hong H, Lee D S, Conkright W, et al. Phase I clinical trial of a recombinant canarypoxvirus (ALVAC) vaccine expressing human carcinoembryonic antigen and the B7.1 co-stimulatory molecule. Cancer Immunol. Immunother. 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin. Cancer. Res. 2000; 6:2219-28; Musey L, Ding Y, Elizaga M, et al. HIV-1 vaccination administered intramuscularly can induce both systemic and mucosal T cell immunity in HIV-1-uninfected individuals. J. Immunol. 2003; 171:1094-101; Paoletti E. Applications of pox virus vectors to vaccination: an update. Proc. Natl. Acad. Sci. USA 1996; 93:11349-53; U.S. Pat. No. 7,255,862). In a phase I clinical trial, an ALVAC virus expressing the tumor antigen CEA showed an excellent safety profile and resulted in increased CEA-specific T cell responses in selected patients; objective clinical responses, however, were not observed (Marshall J L, Hawkins M J, Tsang K Y, et al. Phase I study in cancer patients of a replication-defective avipox recombinant vaccine that expresses human carcinoembryonic antigen. J. Clin. Oncol. 1999; 17:332-7).

In some embodiments, a Modified Vaccinia Ankara (MVA) virus may be used as a viral vector for an antigen vaccine or immunogenic composition. MVA is a member of the Orthopoxvirus family and has been generated by about 570 serial passages on chicken embryo fibroblasts of the Ankara strain of Vaccinia virus (CVA) (see, e.g., Mayr, A., et al., Infection 3, 6-14, 1975). As a consequence of these passages, the resulting MVA virus contains 31 kilobases less genomic information compared to CVA, and is highly host cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol. Immunother. January 2012; 61(1): 19-29). Methods to make and use recombinant MVA has been described (e.g., see U.S. Pat. Nos. 8,309,098 and 5,185,146 hereby incorporated in its entirety).

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

Host cells are genetically engineered (transduced or transformed or transfected) with the vectors which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants or amplifying the polynucleotides. The culture conditions, such as temperature, pH and the like, are those previously used with the host cell selected for expression, and will be apparent to the ordinarily skilled artisan.

As representative examples of appropriate hosts, there can be mentioned: bacterial cells, such as E. coli, Bacillus subtilis, Salmonella typhimurium and various species within the genera Pseudomonas, Streptomyces, and Staphylococcus; fungal cells, such as yeast; insect cells such as Drosophila and Sf9; animal cells such as COS-7 lines of monkey kidney fibroblasts, described by Gluzman, Cell 23:175 (1981), and other cell lines capable of expressing a compatible vector, for example, the C127, 3T3, CHO, HeLa and BHK cell lines or Bowes melanoma; plant cells, etc. The selection of an appropriate host is deemed to be within the scope of those skilled in the art from the teachings herein.

Yeast, insect or mammalian cell hosts can also be used, employing suitable vectors and control sequences. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines.

Polynucleotides described herein can be administered and expressed in human cells (e.g., immune cells, including dendritic cells). A human codon usage table can be used to guide the codon choice for each amino acid. Such polynucleotides comprise spacer amino acid residues between epitopes and/or analogs, such as those described above, or can comprise naturally-occurring flanking sequences adjacent to the epitopes and/or analogs (and/or CTL (e.g., CD8⁺), Th (e.g., CD4⁺), and B cell epitopes).

Standard regulatory sequences well known to those of skill in the art can be included in the vector to ensure expression in the human target cells. Several vector elements are desirable: a promoter with a downstream cloning site for polynucleotide, e.g., minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences. In some embodiments, the promoter is the CMV-IE promoter.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR1, pBR322, pMB9 and their derivatives, wider host range plasmids, such as M13 and filamentous single-stranded DNA phages.

Vectors may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (IM) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Sharei et al., Ex Vivo Cytosolic Delivery of Functional Macromolecules to Immune Cells, PLOS ONE (2015)).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5: 505-10 (1991)). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol.

In some embodiments, a vector comprises a polynucleotide encoding a first peptide comprising a first neoepitope and a second peptide comprising a second neoepitope. In some embodiments, the first and second peptides are derived from the same protein. The at least two distinct peptides may vary by length, amino acid sequence or both. The peptides are derived from any protein known to or have been found to contain a tumor specific mutation. In some embodiments, a vector comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, a vector comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof.

In some embodiments, a vector comprises a polynucleotide operably linked to a promoter. In some embodiments, the vector is a self-amplifying RNA replicon, plasmid, phage, transposon, cosmid, virus, or virion. In some embodiments, the vector is derived from a retrovirus, lentivirus, adenovirus, adeno-associated virus, herpes virus, pox virus, alpha virus, vaccinia virus, hepatitis B virus, human papillomavirus or a pseudotype thereof. In some embodiments, the vector is a non-viral vector. In some embodiments, the non-viral vector is a nanoparticle, a cationic lipid, a cationic polymer, a metallic nanopolymer, a nanorod, a liposome, a micelle, a microbubble, a cell-penetrating peptide, or a liposphere.

### T Cell Receptors

In one aspect, the present disclosure provides cells expressing a neoantigen-recognizing receptor that activates an immunoresponsive cell (e.g., T cell receptor (TCR) or chimeric antigen receptor (CAR)), and methods of using such cells for the treatment of a disease that requires an enhanced immune response. Such cells include genetically modified immunoresponsive cells (e.g., T cells, Natural Killer (NK) cells, cytotoxic T lymphocytes (CTL (e.g., CD8⁺)) cells, helper T lymphocyte (Th (e.g., CD4⁺)) cells) expressing an antigen-recognizing receptor (e.g., TCR or CAR) that binds one of the neoantigenic peptides described herein, and methods of use therefore for the treatment of neoplasia and other pathologies where an increase in an antigen-specific immune response is desired. T cell activation is mediated by a TCR or a CAR targeted to an antigen.

The present disclosure provides cells expressing a combination of an antigen-recognizing receptor that activates an immunoresponsive cell (e.g., TCR, CAR) and a chimeric co-stimulating receptor (CCR), and methods of using such cells for the treatment of a disease that requires an enhanced immune response. In some embodiments, tumor antigen-specific T cells, NK cells, CTL cells or other immunoresponsive cells are used as shuttles for the selective enrichment of one or more co-stimulatory ligands for the treatment or prevention of neoplasia. Such cells are administered to a human subject in need thereof for the treatment or prevention of a particular cancer.

In some embodiments, the tumor antigen-specific human lymphocytes that can be used in the methods of the present disclosure include, without limitation, peripheral donor lymphocytes genetically modified to express chimeric antigen receptors (CARs) (Sadelain, M., et al. 2003 Nat Rev Cancer 3:35-45), peripheral donor lymphocytes genetically modified to express a full-length tumor antigen-recognizing T cell receptor complex comprising the a and p heterodimer (Morgan, R. A., et al. 2006 Science 314:126-129), lymphocyte cultures derived from tumor infiltrating lymphocytes (TILs) in tumor biopsies (Panelli, M. C., et al. 2000 J Immunol 164:495-504; Panelli, M. C., et al. 2000 J Immunol 164:4382-4392), and selectively in vitro-expanded antigen-specific peripheral blood leukocytes employing artificial antigen-presenting cells (AAPCs) or pulsed dendritic cells (Dupont, J., et al. 2005 Cancer Res 65:5417-5427; Papanicolaou, G. A., et al. 2003 Blood 102:2498-2505). The T cells may be autologous, allogeneic, or derived in vitro from engineered progenitor or stem cells.

In some embodiments, the immunotherapeutic is an engineered receptor. In some embodiments, the engineered receptor is a chimeric antigen receptor (CAR), a T cell receptor (TCR), or a B-cell receptor (BCR), an adoptive T cell therapy (ACT), or a derivative thereof. In other aspects, the engineered receptor is a chimeric antigen receptor (CAR). In some aspects, the CAR is a first generation CAR. In other aspects, the CAR is a second generation CAR. In still other aspects, the CAR is a third generation CAR. In some aspects, the CAR comprises an extracellular portion, a transmembrane portion, and an intracellular portion. In some aspects, the intracellular portion comprises at least one T cell co-stimulatory domain. In some aspects, the T cell co-stimulatory domain is selected from the group consisting of CD27, CD28, TNFRS9 (4-1BB), TNFRSF4 (OX40), TNFRSF8 (CD30), CD40LG (CD40L), ICOS, ITGB2 (LFA-1), CD2, CD7, KLRC2 (NKG2C), TNFRS18 (GITR), TNFRSF14 (HVEM), or any combination thereof.

In some aspects, the engineered receptor binds a target. In some aspects, the binding is specific to a peptide specific to one or more subjects suffering from a disease or condition.

In some aspects, the immunotherapeutic is a cell as described in detail herein. In some aspects, the immunotherapeutic is a cell comprising a receptor that specifically binds a peptide or neoepitope described herein. In some aspects, the immunotherapeutic is a cell used in combination with the peptides/nucleic acids of the present disclosure. In some embodiments, the cell is a patient cell. In some embodiments, the cell is a T cell. In some embodiments, the cell is tumor infiltrating lymphocyte.

In some aspects, a subject with a condition or disease is treated based on a T cell receptor repertoire of the subject. In some embodiments, a peptide or neoepitope is selected based on a T cell receptor repertoire of the subject. In some embodiments, a subject is treated with T cells expressing TCRs specific to a peptide or neoepitope as described herein. In some embodiments, a subject is treated with a peptide or neoepitope specific to TCRs, e.g., subject specific TCRs. In some embodiments, a subject is treated with a peptide or neoepitope specific to T cells expressing TCRs, e.g., subject specific TCRs. In some embodiments, a subject is treated with a peptide or neoepitope specific to subject specific TCRs.

In some embodiments, the composition as described herein is selected based on TCRs identified in one or more subjects. In some embodiments, identification of a T cell repertoire and testing in functional assays is used to determine the composition to be administered to one or more subjects with a condition or disease. In some embodiments, the composition is an antigen vaccine comprising one or more peptides or proteins as described herein. In some embodiments, the vaccine comprises subject specific neoantigenic peptides. In some embodiments, the peptides to be included in the vaccine are selected based on a quantification of subject specific TCRs that bind to the neoepitopes. In some embodiments, the peptides are selected based on a binding affinity of the peptide to a TCR. In some embodiments, the selecting is based on a combination of both the quantity and the binding affinity. For example, a TCR that binds strongly to a neoepitope in a functional assay, but that is not highly represented in a TCR repertoire may be a good candidate for an antigen vaccine because T cells expressing the TCR would be advantageously amplified.

In some embodiments, the peptide or protein is selected for administering to one or more subjects based on binding to TCRs. In some embodiments, T cells, such as T cells from a subject with a disease or condition, can be expanded. Expanded T cells that express TCRs specific to a neoantigenic peptide or neoepitope can be administered back to a subject. In some embodiments, suitable cells, e.g., PBMCs, are transduced or transfected with polynucleotides for expression of TCRs specific to a neoantigenic peptide or neoepitope and administered to a subject. T cells expressing TCRs specific to a neoantigenic peptide or neoepitope can be expanded and administered back to a subject. In some embodiments, T cells that express TCRs specific to a neoantigenic peptide or neoepitope that result in cytolytic activity when incubated with autologous diseased tissue can be expanded and administered to a subject. In some embodiments, T cells used in functional assays result in binding to a neoantigenic peptide or neoepitope can be expanded and administered to a subject. In some embodiments, TCRs that have been determined to bind to subject specific neoantigenic peptides or neoepitopes can be expressed in T cells and administered to a subject.

In an embodiment, the present disclosure provides a composition comprising a first peptide comprising a first neoepitope and a second peptide comprising a second neoepitope, wherein the first peptide is different from the second peptide, and wherein the first neoepitope comprises a mutation and the second neoepitope comprises the same mutation. In some embodiments, the composition as provided herein comprises a first T cell comprising a first T cell receptor (TCR) specific for the first neoepitope and a second T cell comprising a second TCR specific for the second neoepitope. In some embodiments, the first and second peptides are derived from the same protein.

In another embodiment, the present disclosure provides a composition comprising a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the composition as provided herein comprises a first T cell comprising a first T cell receptor (TCR) specific for the first neoepitope and a second T cell comprising a second TCR specific for the second neoepitope. In some embodiments, the first mutation and the second mutation are the same.

In some embodiments, the first neoepitope binds to a class I HLA protein to form a class I HLA-peptide complex. In some embodiments, the first neoepitope binds to a class II HLA protein to form a class II HLA-peptide complex. In some embodiments, the second neoepitope binds to a class II HLA a protein to form a class II HLA-peptide complex. In some embodiments, the second neoepitope binds to a class I HLA protein to form a class I HLA-peptide complex. In some embodiments, the first neoepitope activates CD8⁺ T cells. In some embodiments, the first neoepitope activates CD4⁺ T cells. In some embodiments, the second neoepitope activates CD4⁺ T cells. In some embodiments, the second neoepitope activates CD8⁺ T cells. In some embodiments, a TCR of a CD4⁺ T cell binds to a class II HLA-peptide complex. In some embodiments, a TCR of a CD8⁺ T cell binds to a class II HLA-peptide complex. In some embodiments, a TCR of a CD8⁺ T cell binds to a class I HLA-peptide complex. In some embodiments, a TCR of a CD4⁺ T cell binds to a class I HLA-peptide complex.

In some embodiments, the first TCR is a first chimeric antigen receptor specific for the first neoepitope and the second TCR is a second chimeric antigen receptor specific for the second neoepitope. In some embodiments, the first T cell is a cytotoxic T cell. In some embodiments, the first T cell is a gamma delta T cell. In some embodiments, the second T cell is a helper T cell. In some embodiments, the first and/or second TCR binds to an HLA-peptide complex with a K_{D} or an IC₅₀ of less than 1,000 nM, 900 nM, 800 nM, 700 nM, 600 nM, 500 nM, 250 nM, 150 nM, 100 nM, 50 nM, 25 nM or 10 nM. In some embodiments, the first and/or second TCR binds to an HLA class I-peptide complex with a K_{D} or an IC₅₀ of less than 1,000 nM, 900 nM, 800 nM, 700 nM, 600 nM, 500 nM, 250 nM, 150 nM, 100 nM, 50 nM, 25 nM or 10 nM. In some embodiments, the first and/or second TCR binds to an HLA class II-peptide complex with a K_{D} or an IC₅₀ of less than 2,000, 1,500, 1,000 nM, 900 nM, 800 nM, 700 nM, 600 nM, 500 nM, 250 nM, 150 nM, 100 nM, 50 nM, 25 nM or 10 nM.

### Antigen Presenting Cells

The neoantigenic peptide or protein can be provided as antigen presenting cells (e.g., dendritic cells) containing such peptides, proteins or polynucleotides as described herein. In other embodiments, such antigen presenting cells are used to stimulate T cells for use in patients. Thus, one embodiment of the present disclosure is a composition containing at least one antigen presenting cell (e.g., a dendritic cell) that is pulsed or loaded with one or more neoantigenic peptides or polynucleotides described herein. In some embodiments, such APCs are autologous (e.g., autologous dendritic cells). Alternatively, peripheral blood mononuclear cells (PBMCs) isolated from a patient can be loaded with neoantigenic peptides or polynucleotides ex vivo. In related embodiments, such APCs or PBMCs are injected back into the patient. In some embodiments, the antigen presenting cells are dendritic cells. In related embodiments, the dendritic cells are autologous dendritic cells that are pulsed with the neoantigenic peptide or nucleic acid. The neoantigenic peptide can be any suitable peptide that gives rise to an appropriate T cell response. T cell therapy using autologous dendritic cells pulsed with peptides from a tumor associated antigen is disclosed in Murphy et al. (1996) The Prostate 29, 371-380 and Tjua et al. (1997) The Prostate 32, 272-278. In some embodiments, the T cell is a CTL (e.g., CD8⁺). In some embodiments, the T cell is a helper T lymphocyte (Th (e.g., CD4⁺)).

In some embodiments, the present disclosure provides a composition comprising a cell-based immunogenic pharmaceutical composition that can also be administered to a subject. For example, an antigen presenting cell (APC) based immunogenic pharmaceutical composition can be formulated using any of the well-known techniques, carriers, and excipients as suitable and as understood in the art. APCs include monocytes, monocyte-derived cells, macrophages, and dendritic cells. Sometimes, an APC based immunogenic pharmaceutical composition can be a dendritic cell-based immunogenic pharmaceutical composition.

A dendritic cell-based immunogenic pharmaceutical composition can be prepared by any methods well known in the art. In some cases, dendritic cell-based immunogenic pharmaceutical compositions can be prepared through an ex vivo or in vivo method. The ex vivo method can comprise the use of autologous DCs pulsed ex vivo with the polypeptides described herein, to activate or load the DCs prior to administration into the patient. The in vivo method can comprise targeting specific DC receptors using antibodies coupled with the polypeptides described herein. The DC-based immunogenic pharmaceutical composition can further comprise DC activators such as TLR3, TLR-7-8, and CD40 agonists. The DC-based immunogenic pharmaceutical composition can further comprise adjuvants, and a pharmaceutically acceptable carrier.

Antigen presenting cells (APCs) can be prepared from a variety of sources, including human and non-human primates, other mammals, and vertebrates. In certain embodiments, APCs can be prepared from blood of a human or non-human vertebrate. APCs can also be isolated from an enriched population of leukocytes. Populations of leukocytes can be prepared by methods known to those skilled in the art. Such methods typically include collecting heparinized blood, apheresis or leukopheresis, preparation of buffy coats, rosetting, centrifugation, density gradient centrifugation (e.g., using Ficoll, colloidal silica particles, and sucrose), differential lysis non-leukocyte cells, and filtration. A leukocyte population can also be prepared by collecting blood from a subject, defibrillating to remove the platelets and lysing the red blood cells. The leukocyte population can optionally be enriched for monocytic dendritic cell precursors.

Blood cell populations can be obtained from a variety of subjects, according to the desired use of the enriched population of leukocytes. The subject can be a healthy subject. Alternatively, blood cells can be obtained from a subject in need of immunostimulation, such as, for example, a cancer patient or other patient for which immunostimulation will be beneficial. Likewise, blood cells can be obtained from a subject in need of immune suppression, such as, for example, a patient having an autoimmune disorder (e.g., rheumatoid arthritis, diabetes, lupus, multiple sclerosis, and the like). A population of leukocytes also can be obtained from an HLA-matched healthy individual.

When blood is used as a source of APC, blood leukocytes may be obtained using conventional methods that maintain their viability. According to one aspect of the present disclosure, blood can be diluted into medium that may or may not contain heparin or other suitable anticoagulant. The volume of blood to medium can be about 1 to 1. Cells can be concentrated by centrifugation of the blood in medium at about 1,000 rpm (150 g) at 4° C. Platelets and red blood cells can be depleted by resuspending the cells in any number of solutions known in the art that will lyse erythrocytes, for example ammonium chloride. For example, the mixture may be medium and ammonium chloride at about 1:1 by volume. Cells may be concentrated by centrifugation and washed in the desired solution until a population of leukocytes, substantially free of platelets and red blood cells, is obtained. Any isotonic solution commonly used in tissue culture may be used as the medium for separating blood leukocytes from platelets and red blood cells. Examples of such isotonic solutions can be phosphate buffered saline, Hanks balanced salt solution, and complete growth media. APCs and/or APC precursor cells may also purified by elutriation.

In one embodiment, the APCs can be non-nominal APCs under inflammatory or otherwise activated conditions. For example, non-nominal APCs can include epithelial cells stimulated with interferon-gamma, T cells, B cells, and/or monocytes activated by factors or conditions that induce APC activity. Such non-nominal APCs can be prepared according to methods known in the art.

The APCs can be cultured, expanded, differentiated and/or, matured, as desired, according to the according to the type of APC. The APCs can be cultured in any suitable culture vessel, such as, for example, culture plates, flasks, culture bags, and bioreactors.

In certain embodiments, APCs can be cultured in suitable culture or growth medium to maintain and/or expand the number of APCs in the preparation. The culture media can be selected according to the type of APC isolated. For example, mature APCs, such as mature dendritic cells, can be cultured in growth media suitable for their maintenance and expansion. The culture medium can be supplemented with amino acids, vitamins, antibiotics, divalent cations, and the like. In addition, cytokines, growth factors and/or hormones, can be included in the growth media. For example, for the maintenance and/or expansion of mature dendritic cells, cytokines, such as granulocyte/macrophage colony stimulating factor (GM-CSF) and/or interleukin 4 (IL-4), can be added. In other embodiments, immature APCs can be cultured and/or expanded. Immature dendritic cells can they retain the ability to uptake target mRNA and process new antigen. In some embodiments, immature dendritic cells can be cultured in media suitable for their maintenance and culture. The culture medium can be supplemented with amino acids, vitamins, antibiotics, divalent cations, and the like. In addition, cytokines, growth factors and/or hormones, can be included in the growth media.

Other immature APCs can similarly be cultured or expanded. Preparations of immature APCs can be matured to form mature APCs. Maturation of APCs can occur during or following exposure to the neoantigenic peptides. In certain embodiments, preparations of immature dendritic cells can be matured. Suitable maturation factors include, for example, cytokines TNF-α, bacterial products (e.g., BCG), and the like. In another aspect, isolated APC precursors can be used to prepare preparations of immature APCs. APC precursors can be cultured, differentiated, and/or matured. In certain embodiments, monocytic dendritic cell precursors can be cultured in the presence of suitable culture media supplemented with amino acids, vitamins, cytokines, and/or divalent cations, to promote differentiation of the monocytic dendritic cell precursors to immature dendritic cells. In some embodiments, the APC precursors are isolated from PBMCs. The PBMCs can be obtained from a donor, for example, a human donor, and can be used freshly or frozen for future usage. In some embodiments, the APC is prepared from one or more APC preparations. In some embodiments, the APC comprises an APC loaded with the first and second neoantigenic peptides comprising the first and second neoepitopes or polynucleotides encoding the first and second neoantigenic peptides comprising the first and second neoepitopes. In some embodiments, the APC is an autologous APC, an allogenic APC, or an artificial APC.

In an embodiment, the present disclosure provides a composition comprising an APC comprising a first peptide comprising a first neoepitope and a second peptide comprising a second neoepitope, wherein the first peptide is different from the second peptide, and wherein the first neoepitope comprises a mutation and the second neoepitope comprises the same mutation. In some embodiments, the first and second peptides are derived from the same protein. In another embodiment, the present disclosure provides a composition comprising an APC comprising a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same.

### Adjuvants

An adjuvant can be used to enhance the immune response (humoral and/or cellular) elicited in a patient receiving a composition as provided herein. Sometimes, adjuvants can elicit a Th1-type response. Other times, adjuvants can elicit a Th2-type response. A Th1-type response can be characterized by the production of cytokines such as IFN-γ as opposed to a Th2-type response which can be characterized by the production of cytokines such as IL-4, IL-5 and IL-10.

In some aspects, lipid-based adjuvants, such as MPLA and MDP, can be used with the immunogenic pharmaceutical compositions disclosed herein. Monophosphoryl lipid A (MPLA), for example, is an adjuvant that causes increased presentation of liposomal antigen to specific T Lymphocytes. In addition, a muramyl dipeptide (MDP) can also be used as a suitable adjuvant in conjunction with the immunogenic pharmaceutical formulations described herein.

Suitable adjuvants are known in the art (see, WO 2015/095811) and include, but are not limited to poly(I:C), poly-ICLC, Hiltonol, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®. vector system, PLG microparticles, resiquimod, SRL172, virosomes and other virus-like particles, YF-17D, VEGF trap, R848, beta-glucan, Pam3Cys, Pam3CSK4, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants also include incomplete Freund's or GM-CSF. Several immunological adjuvants (e.g., MF59) specific for dendritic cells and their preparation have been described previously (Dupuis M, et al., Cell Immunol. 1998; 186(1):18-27; Allison A C; Dev. Biol. Stand. 1998; 92:3-11) (Mosca et al. Frontiers in Bioscience, 2007; 12:4050-4060) (Gamvrellis et al. Immunol & Cell Biol. 2004; 82: 506-516). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, PGE1, PGE2, IL-1, IL-1b, IL-4, IL-6 and CD40L) (U.S. Pat. No. 5,849,589 incorporated herein by reference in its entirety) and acting as immunoadjuvants (e.g., IL-12) (Gabrilovich D I, et al., J. Immunother. Emphasis Tumor Immunol. 1996 (6):414-418).

Adjuvant can also comprise stimulatory molecules such as cytokines. Non-limiting examples of cytokines include: CCL20, α-interferon (IFN-α), β-interferon (IFN-β), γ-interferon, platelet derived growth factor (PDGF), TNFα, TNFβ (lymphotoxin alpha (LTα)), GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-la, MIP-1-, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-I, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAPI, and TAP2.

Additional adjuvants include: MCP-1, MIP-la, MIP-lp, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-1, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some aspects, an adjuvant can be a modulator of a toll like receptor. Examples of modulators of toll-like receptors include TLR-9 agonists and are not limited to small molecule modulators of toll-like receptors such as Imiquimod. Other examples of adjuvants that are used in combination with an immunogenic pharmaceutical composition described herein can include and are not limited to saponin, CpG ODN and the like. Sometimes, an adjuvant is selected from bacteria toxoids, polyoxypropylene-polyoxyethylene block polymers, aluminum salts, liposomes, CpG polymers, oil-in-water emulsions, or a combination thereof. Sometimes, an adjuvant is an oil-in-water emulsion. The oil-in-water emulsion can include at least one oil and at least one surfactant, with the oil(s) and surfactant(s) being biodegradable (metabolisable) and biocompatible. The oil droplets in the emulsion can be less than 5 μm in diameter, and can even have a sub-micron diameter, with these small sizes being achieved with a microfluidiser to provide stable emulsions. Droplets with a size less than 220 nm can be subjected to filter sterilization.

### Methods of Treatment and Pharmaceutical Compositions

The neoantigen therapeutics (e.g., peptides, polynucleotides, TCR, CAR, cells containing TCR or CAR, APC or dendritic cell containing polypeptide, dendritic cell containing polynucleotide, antibody, etc.) described herein are useful in a variety of applications including, but not limited to, therapeutic treatment methods, such as the treatment of cancer. In some embodiments, the therapeutic treatment methods comprise immunotherapy. In certain embodiments, a neoantigenic peptide is useful for activating, promoting, increasing, and/or enhancing an immune response, redirecting an existing immune response to a new target, increasing the immunogenicity of a tumor, inhibiting tumor growth, reducing tumor volume, increasing tumor cell apoptosis, and/or reducing the tumorigenicity of a tumor. The methods of use can be in vitro, ex vivo, or in vivo methods.

In some aspects, the present disclosure provides methods for activating an immune response in a subject using a neoantigenic peptide or protein described herein. In some embodiments, the present disclosure provides methods for promoting an immune response in a subject using a neoantigenic peptide described herein. In some embodiments, the present disclosure provides methods for increasing an immune response in a subject using a neoantigenic peptide described herein. In some embodiments, the present disclosure provides methods for enhancing an immune response using a neoantigenic peptide. In some embodiments, the activating, promoting, increasing, and/or enhancing of an immune response comprises increasing cell-mediated immunity. In some embodiments, the activating, promoting, increasing, and/or enhancing of an immune response comprises increasing T cell activity or humoral immunity. In some embodiments, the activating, promoting, increasing, and/or enhancing of an immune response comprises increasing CTL or Th activity. In some embodiments, the activating, promoting, increasing, and/or enhancing of an immune response comprises increasing NK cell activity. In some embodiments, the activating, promoting, increasing, and/or enhancing of an immune response comprises increasing T cell activity and increasing NK cell activity. In some embodiments, the activating, promoting, increasing, and/or enhancing of an immune response comprises increasing CTL activity and increasing NK cell activity. In some embodiments, the activating, promoting, increasing, and/or enhancing of an immune response comprises inhibiting or decreasing the suppressive activity of T regulatory (Treg) cells. In some embodiments, the immune response is a result of antigenic stimulation. In some embodiments, the antigenic stimulation is a tumor cell. In some embodiments, the antigenic stimulation is cancer.

In some embodiments, the present disclosure provides methods of activating, promoting, increasing, and/or enhancing of an immune response using a neoantigenic peptide described herein. In some embodiments, a method comprises administering to a subject in need thereof a therapeutically effective amount of a neoantigenic peptide that delivers a neoantigenic peptide or polynucleotide to a tumor cell. In some embodiments, a method comprises administering to a subject in need thereof a therapeutically effective amount of a neoantigenic peptide internalized by the tumor cell. In some embodiments, a method comprises administering to a subject in need thereof a therapeutically effective amount of a neoantigenic peptide that is internalized by a tumor cell, and the neoantigenic peptide is processed by the cell. In some embodiments, a method comprises administering to a subject in need thereof a therapeutically effective amount of a neoantigenic polypeptide that is internalized by a tumor cell and a neoepitope is presented on the surface of the tumor cell. In some embodiments, a method comprises administering to a subject in need thereof a therapeutically effective amount of a neoantigenic polypeptide that is internalized by the tumor cell, is processed by the cell, and an antigenic peptide is presented on the surface of the tumor cell.

In some embodiments, a method comprises administering to a subject in need thereof a therapeutically effective amount of a neoantigenic peptide or polynucleotide described herein that delivers an exogenous polypeptide comprising at least one neoantigenic peptide to a tumor cell, wherein at least one neoepitope derived from the neoantigenic peptide is presented on the surface of the tumor cell. In some embodiments, the antigenic peptide is presented on the surface of the tumor cell in complex with a MHC class I molecule. In some embodiments, the neoepitope is presented on the surface of the tumor cell in complex with a MHC class II molecule.

In some embodiments, a method comprises contacting a tumor cell with a neoantigenic polypeptide or polynucleotide described herein that delivers an exogenous polypeptide comprising at least one neoantigenic peptide to the tumor cell, wherein at least one neoepitope derived from the at least one neoantigenic peptide is presented on the surface of the tumor cell. In some embodiments, the neoepitope is presented on the surface of the tumor cell in complex with a MHC class I molecule. In some embodiments, the neoepitope is presented on the surface of the tumor cell in complex with a MHC class II molecule.

In some embodiments, a method comprises administering to a subject in need thereof a therapeutically effective amount of a neoantigenic polypeptide or polynucleotide described herein that delivers an exogenous polypeptide comprising at least one antigenic peptide to a tumor cell, wherein the neoepitope is presented on the surface of the tumor cell, and an immune response against the tumor cell is induced. In some embodiments, the immune response against the tumor cell is increased. In some embodiments, the neoantigenic polypeptide or polynucleotide delivers an exogenous polypeptide comprising at least one neoantigenic peptide to a tumor cell, wherein the neoepitope is presented on the surface of the tumor cell, and tumor growth is inhibited.

In some embodiments, a method comprises administering to a subject in need thereof a therapeutically effective amount of a neoantigenic polypeptide or polynucleotide described herein that delivers an exogenous polypeptide comprising at least one neoantigenic peptide to a tumor cell, wherein the neoepitope derived from the at least one neoantigenic peptide is presented on the surface of the tumor cell, and T cell killing directed against the tumor cell is induced. In some embodiments, T cell killing directed against the tumor cell is enhanced. In some embodiments, T cell killing directed against the tumor cell is increased.

In some embodiments, a method of increasing an immune response in a subject comprises administering to the subject a therapeutically effective amount of a neoantigenic therapeutic described herein, wherein the agent is an antibody that specifically binds the neoantigen described herein. In some embodiments, a method of increasing an immune response in a subject comprises administering to the subject a therapeutically effective amount of the antibody.

The present disclosure provides methods of redirecting an existing immune response to a tumor. In some embodiments, a method of redirecting an existing immune response to a tumor comprises administering to a subject a therapeutically effective amount of a neoantigen therapeutic described herein. In some embodiments, the existing immune response is against a virus. In some embodiments, the virus is selected from the group consisting of: measles virus, varicella-zoster virus (VZV; chickenpox virus), influenza virus, mumps virus, poliovirus, rubella virus, rotavirus, hepatitis A virus (HAV), hepatitis B virus (HBV), Epstein Barr virus (EBV), and cytomegalovirus (CMV). In some embodiments, the virus is varicella-zoster virus. In some embodiments, the virus is cytomegalovirus. In some embodiments, the virus is measles virus. In some embodiments, the existing immune response has been acquired after a natural viral infection. In some embodiments, the existing immune response has been acquired after vaccination against a virus. In some embodiments, the existing immune response is a cell-mediated response. In some embodiments, the existing immune response comprises cytotoxic T cells (CTLs) or Th cells.

In some embodiments, a method of redirecting an existing immune response to a tumor in a subject comprises administering a fusion protein comprising (i) an antibody that specifically binds a neoantigen and (ii) at least one neoantigenic peptide described herein, wherein (a) the fusion protein is internalized by a tumor cell after binding to the tumor-associated antigen or the neoepitope; (b) the neoantigenic peptide is processed and presented on the surface of the tumor cell associated with a MHC class I molecule; and (c) the neoantigenic peptide/MHC Class I complex is recognized by cytotoxic T cells. In some embodiments, the cytotoxic T cells are memory T cells. In some embodiments, the memory T cells are the result of a vaccination with the neoantigenic peptide.

The present disclosure provides methods of increasing the immunogenicity of a tumor. In some embodiments, a method of increasing the immunogenicity of a tumor comprises contacting a tumor or tumor cells with an effective amount of a neoantigen therapeutic described herein. In some embodiments, a method of increasing the immunogenicity of a tumor comprises administering to a subject a therapeutically effective amount of a neoantigen therapeutic described herein.

The present disclosure also provides methods for inhibiting growth of a tumor using a neoantigen therapeutic described herein. In certain embodiments, a method of inhibiting growth of a tumor comprises contacting a cell mixture with a neoantigen therapeutic in vitro. For example, an immortalized cell line or a cancer cell line mixed with immune cells (e.g., T cells) is cultured in medium to which a neoantigenic peptide is added. In some embodiments, tumor cells are isolated from a patient sample, for example, a tissue biopsy, pleural effusion, or blood sample, mixed with immune cells (e.g., T cells), and cultured in medium to which a neoantigen therapeutic is added. In some embodiments, a neoantigen therapeutic increases, promotes, and/or enhances the activity of the immune cells. In some embodiments, a neoantigen therapeutic inhibits tumor cell growth. In some embodiments, a neoantigen therapeutic activates killing of the tumor cells.

In certain embodiments, the subject is a human. In certain embodiments, the subject has a tumor or the subject had a tumor which was at least partially removed.

In some embodiments, a method of inhibiting growth of a tumor comprises redirecting an existing immune response to a new target, comprising administering to a subject a therapeutically effective amount of a neoantigen therapeutic, wherein the existing immune response is against an antigenic peptide delivered to the tumor cell by the neoantigenic peptide. In some embodiments, the method of treatment involves a step of identifying one or more HLA subtypes expressed in the subject before administrating a peptide, such that the peptide binds to at least one or more HLA subtype specifically expressed by the subject. In some embodiments, if one or more mutant BTK peptides selected from Table 34 are administered in a subject, a prior determination of the expression of the HLA subtype corresponding to the peptide from Table 34 is performed in the subject, such that the administered peptide binds to at least one or more HLA subtype specifically expressed by the subject. In some embodiments, the method comprises determining that the subject expresses a protein encoded by HLA-C14:02 allele, HLA-C14:03 allele, HLA-A33:03 allele, HLA-C04:01 allele, HLA-B15:09 allele or HLA-B38:02 allele, wherein the therapeutic comprises a mutant BTK peptide having the amino acid sequence EYMANGSLL. In some embodiments, if the method comprises determining that the subject expresses a protein encoded by any one of HLA-C02:02 allele, HLA-C03:02 allele, HLA-B53:01 allele, HLA-C12:02 allele, HLA-C12:03 allele, HLA-A36:01 allele, HLA-A26:01 allele, HLA-A25:01 allele, HLA-B57:01 allele, HLA-A03:01 allele, HLA-B46:01 allele, HLA-B15:03 allele, HLA-A33:03 allele, HLA-B35:03 allele or a HLA-A11:01 allele, wherein the therapeutic comprises a mutant BTK peptide having the amino acid sequence MANGSLLNY. In some embodiments, the method comprises determining that the subject expresses a protein encoded by any one of HLA-A02:04 allele, HLA-A02:03 allele, HLA-C03:02 allele, HLA-A03:01 allele, HLA-A32:01 allele, HLA-A02:07 allele, HLA-C14:03 allele, HLA-C14:02 allele, HLA-A31:01 allele, HLA-A30:02 allele, HLA-A74:01 allele, HLA-C06:02 allele, HLA-B15:03 allele, HLA-B46:01 allele, HLA-B13:02 allele, HLA-A25:01 allele, HLA-A29:02 allele or a HLA-C01:02 allele, wherein the therapeutic comprises a mutant BTK peptide having the amino acid sequence SLLNYLREM.

In some embodiments, the method comprises determining that the subject expresses a protein encoded by any one of HLA-B14:02 allele, HLA-B49:01 allele, HLA-B44:03 allele, HLA-B44:02 allele, HLA-B37:01 allele, HLA-B15:09 allele, HLA-B41:01 or HLA-B50:01 allele, wherein the therapeutic comprises a mutant BTK peptide having the amino acid sequence TEYMANGSL.

In certain embodiments, the tumor comprises cancer stem cells. In certain embodiments, the frequency of cancer stem cells in the tumor is reduced by administration of the neoantigen therapeutic. In some embodiments, a method of reducing the frequency of cancer stem cells in a tumor in a subject, comprising administering to the subject a therapeutically effective amount of a neoantigen therapeutic is provided.

In addition, in some aspects the present disclosure provides a method of reducing the tumorigenicity of a tumor in a subject, comprising administering to the subject a therapeutically effective amount of a neoantigen therapeutic described herein. In certain embodiments, the tumor comprises cancer stem cells. In some embodiments, the tumorigenicity of a tumor is reduced by reducing the frequency of cancer stem cells in the tumor. In some embodiments, the methods comprise using the neoantigen therapeutic described herein. In certain embodiments, the frequency of cancer stem cells in the tumor is reduced by administration of a neoantigen therapeutic described herein.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a breast tumor. In some embodiments, the tumor is a lung tumor. In certain embodiments, the tumor is a pancreatic tumor. In certain embodiments, the tumor is a melanoma tumor. In some embodiments, the tumor is a solid tumor.

The present disclosure further provides methods for treating cancer in a subject comprising administering to the subject a therapeutically effective amount of a neoantigen therapeutic described herein.

In some embodiments, a method of treating cancer comprises redirecting an existing immune response to a new target, the method comprising administering to a subject a therapeutically effective amount of neoantigen therapeutic, wherein the existing immune response is against an antigenic peptide delivered to the cancer cell by the neoantigenic peptide.

The present disclosure provides for methods of treating cancer comprising administering to a subject a therapeutically effective amount of a neoantigen therapeutic described herein (e.g., a subject in need of treatment). In certain embodiments, the subject is a human. In certain embodiments, the subject has a cancerous tumor. In certain embodiments, the subject has had a tumor at least partially removed.

Subjects can be, for example, mammal, humans, pregnant women, elderly adults, adults, adolescents, pre-adolescents, children, toddlers, infants, newborn, or neonates. A subject can be a patient. In some cases, a subject can be a human. In some cases, a subject can be a child (i.e. a young human being below the age of puberty). In some cases, a subject can be an infant. In some cases, the subject can be a formula-fed infant. In some cases, a subject can be an individual enrolled in a clinical study. In some cases, a subject can be a laboratory animal, for example, a mammal, or a rodent. In some cases, the subject can be a mouse. In some cases, the subject can be an obese or overweight subject.

In some embodiments, the subject has previously been treated with one or more different cancer treatment modalities. In some embodiments, the subject has previously been treated with one or more of radiotherapy, chemotherapy, or immunotherapy. In some embodiments, the subject has been treated with one, two, three, four, or five lines of prior therapy. In some embodiments, the prior therapy is a cytotoxic therapy.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma. In some embodiments, the cancer is a solid cancer. In some embodiments, the cancer comprises a solid tumor.

In some embodiments, the cancer is a hematologic cancer. In some embodiment, the cancer is selected from the group consisting of: acute myelogenous leukemia (AML), Hodgkin lymphoma, multiple myeloma, T cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T cell lymphoma (CTCL).

In some embodiments, the neoantigen therapeutic is administered as a combination therapy. Combination therapy with two or more therapeutic agents uses agents that work by different mechanisms of action, although this is not required. Combination therapy using agents with different mechanisms of action can result in additive or synergetic effects. Combination therapy can allow for a lower dose of each agent than is used in monotherapy, thereby reducing toxic side effects and/or increasing the therapeutic index of the agent(s). Combination therapy can decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some instances, an immunogenic pharmaceutical composition can be administered with an additional agent. In some embodiments, the neoantigen therapeutic can be administered with an immunotherapy. The immunotherapy can be, for example, an antibody targeting an immune checkpoint. In some embodiments, the antibody is a bispecific antibody. The choice of the additional agent can depend, at least in part, on the condition being treated. The additional agent can include, for example, a checkpoint inhibitor agent such as an anti-PD1, anti-CTLA4, anti-PD-L1, anti CD40, or anti-TIM3 agent (e.g., an anti-PD1, anti-CTLA4, anti-PD-L1, anti CD40, or anti-TIM3 antibody); or any agents having a therapeutic effect for a pathogen infection (e.g. viral infection), including, e.g., drugs used to treat inflammatory conditions such as an NSAID, e.g., ibuprofen, naproxen, acetaminophen, ketoprofen, or aspirin. For example, the checkpoint inhibitor can be a PD-1/PD-L1 antagonist selected from the group consisting of: nivolumab (ONO-4538/BMS-936558, MDX1 106, OPDIVO), pembrolizumab (MK-3475, KEYTRUDA), pidilizumab (CT-011), and MPDL3280A (ROCHE). As another example, formulations can additionally contain one or more supplements, such as vitamin C, E or other anti-oxidants.

The methods of the disclosure can be used to treat any type of cancer known in the art. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present disclosure is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a neoantigen therapeutic described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that can be administered in combination with the neoantigen therapeutic described herein include chemotherapeutic agents. Thus, in some embodiments, the method or treatment involves the administration of an agent described herein in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, Pa.

Useful classes of chemotherapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in the present disclosure include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6 mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In some embodiments, the additional therapeutic agent is albumin-bound paclitaxel.

In some embodiments, an additional therapeutic agent comprises an agent such as a small molecule. For example, treatment can involve the combined administration of an agent of the present disclosure with a small molecule that acts as an inhibitor against tumor-associated antigens including, but not limited to, EGFR, HER2 (ErbB2), and/or VEGF. In some embodiments, an agent of the present disclosure is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor. In another embodiment, the additional therapeutic agent is chemotherapy or other inhibitors that reduce the number of Treg cells. In certain embodiments, the therapeutic agent is cyclophosphamide or an anti-CTLA4 antibody. In another embodiment, the additional therapeutic reduces the presence of myeloid-derived suppressor cells. In a further embodiment, the additional therapeutic is carbotaxol. In another embodiment, the additional therapeutic agent shifts cells to a T helper 1 response. In a further embodiment, the additional therapeutic agent is ibrutinib.

In some embodiments, an additional therapeutic agent comprises a biological molecule, such as an antibody. For example, treatment can involve the combined administration of an agent of the present disclosure with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

The agents and compositions provided herein may be used alone or in combination with conventional therapeutic regimens such as surgery, irradiation, chemotherapy and/or bone marrow transplantation (autologous, syngeneic, allogeneic or unrelated). A set of tumor antigens can be useful, e.g., in a large fraction of cancer patients.

In some embodiments, at least one or more chemotherapeutic agents may be administered in addition to the composition comprising an immunogenic vaccine. In some embodiments, the one or more chemotherapeutic agents may belong to different classes of chemotherapeutic agents.

Examples of chemotherapy agents include, but are not limited to, alkylating agents such as nitrogen mustards (e.g. mechlorethamine (nitrogen mustard), chlorambucil, cyclophosphamide (Cytoxan®), ifosfamide, and melphalan); nitrosoureas (e.g. N-Nitroso-N-methylurea, streptozocin, carmustine (BCNU), lomustine, and semustine); alkyl sulfonates (e.g. busulfan); tetrazines (e.g. dacarbazine (DTIC), mitozolomide and temozolomide (Temodar®)); aziridines (e.g. thiotepa, mytomycin and diaziquone); and platinum drugs (e.g. cisplatin, carboplatin, and oxaliplatin); non-classical alkylating agents such as procarbazine and altretamine (hexamethylmelamine); anti-metabolite agents such as 5-fluorouracil (5-FU), 6-mercaptopurine (6-MP), capecitabine (Xeloda®), cladribine, clofarabine, cytarabine (Ara-C®), decitabine, floxuridine, fludarabine, nelarabine, gemcitabine (Gemzar®), hydroxyurea, methotrexate, pemetrexed (Alimta®), pentostatin, thioguanine, Vidaza; anti-microtubule agents such as vinca alkaloids (e.g. vincristine, vinblastine, vinorelbine, vindesine and vinflunine); taxanes (e.g. paclitaxel (Taxol®), docetaxel (Taxotere®)); podophyllotoxin (e.g. etoposide and teniposide); epothilones (e.g. ixabepilone (Ixempra®)); estramustine (Emcyt®); anti-tumor antibiotics such as anthracyclines (e.g. daunorubicin, doxorubicin (Adriamycin®, epirubicin, idarubicin); actinomycin-D; and bleomycin; topoisomerase I inhibitors such as topotecan and irinotecan (CPT-11); topoisomerase II inhibitors such as etoposide (VP-16), teniposide, mitoxantrone, novobiocin, merbarone and aclarubicin; corticosteroids such as prednisone, methylprednisolone (Solumedrol®), and dexamethasone (Decadron®); L-asparaginase; bortezomib (Velcade®); immunotherapeutic agents such as rituximab (Rituxan®), alemtuzumab (Campath®), thalidomide, lenalidomide (Revlimid®), BCG, interleukin-2, interferon-alfa and cancer vaccines such as Provenge®; hormone therapeutic agents such as fulvestrant (Faslodex®), tamoxifen, toremifene (Fareston®), anastrozole (Arimidex®), exemestan (Aromasin®), letrozole (Femara®), megestrol acetate (Megace®), estrogens, bicalutamide (Casodex®), flutamide (Eulexin®), nilutamide (Nilandron®), leuprolide (Lupron®) and goserelin (Zoladex®); differentiating agents such as retinoids, tretinoin (ATRA or Atralin®), bexarotene (Targretin®) and arsenic trioxide (Arsenox®); and targeted therapeutic agents such as imatinib (Gleevec®), gefitinib (Iressa®) and sunitinib (Sutent®). In some embodiments, the chemotherapy is a cocktail therapy. Examples of a cocktail therapy includes, but is not limited to, CHOP/R-CHOP (rituxan, cyclophosphamide, hydroxydoxorubicin, vincristine, and prednisone), EPOCH (etoposide, prednisone, vincristine, cyclophosphamide, hydroxydoxorubicin), Hyper-CVAD (cyclophosphamide, vincristine, hydroxydoxorubicin, dexamethasone), FOLFOX (fluorouracil (5-FU), leucovorin, oxaliplatin), ICE (ifosfamide, carboplatin, etoposide), DHAP (high-dose cytarabine [ara-C], dexamethasone, cisplatin), ESHAP (etoposide, methylprednisolone, cytarabine [ara-C], cisplatin) and CMF (cyclophosphamide, methotrexate, fluouracil).

In some embodiments, the immunogenic vaccine may be used in combination with an inhibitor of a phosphoinositide 3-kinase (PI3 kinase, PI3K). For example, the immunogenic vaccine may be used in combination with Wortnannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477, AEZS-136 or any combination thereof.

In some embodiments, doses of the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, employed for human treatment can be in the range of about 0.01 mg/kg to about 100 mg/kg per day (e.g., about 0.1 mg/kg to about 100 mg/kg per day, about 0.1 mg/kg to about 50 mg/kg per day, about 10 mg/kg per day or about 30 mg/kg per day). The desired dose may be conveniently administered in a single dose, or as multiple doses administered at appropriate intervals, for example as two, three, four or more sub-doses per day.

In some embodiments, the dosage of the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, may be from about 1 ng/kg to about 100 mg/kg. The dosage of the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, may be at any dosage including, but not limited to, about 1 μg/kg, 25 μg/kg, 50 μg/kg, 75 μg/kg, 100 μμg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

The mode of administration of the immunogenic vaccine and the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, may be simultaneously or sequentially, wherein the immunogenic vaccine and the at least one additional pharmaceutically active agent are sequentially (or separately) administered. For example, the immunogenic vaccine and the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, may be provided in a single unit dosage form for being taken together or as separate entities (e.g. in separate containers) to be administered simultaneously or with a certain time difference. This time difference may be between 1 hour and 1 month, e.g., between 1 day and 1 week, e.g., 48 hours and 3 days. In addition, it is possible to administer the immunogenic vaccine via another administration way than the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136. For example, it may be advantageous to administer either the immunogenic vaccine or the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, intravenously and the other systemically or orally. For example, the immunogenic vaccine is administered intravenously or subcutaneously and the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, orally.

In some embodiments, the immunogenic vaccine is administered chronologically before the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136. In some embodiments, the immunogenic vaccine is administered from 1-24 hours, 2-24 hours, 3-24 hours, 4-24 hours, 5-24 hours, 6-24 hours, 7-24 hours, 8-24 hours, 9-24 hours, 10-24 hours, 11-24 hours, 12-24 hours, 1-30 days, 2-30 days, 3-30 days, 4-30 days, 5-30 days, 6-30 days, 7-30 days, 8-30 days, 9,-30 days, 10-30 days, 11-30 days, 12-30 days, 13-30 days, 14-30 days, 15-30 days, 16-30 days, 17-30 days, 18-30 days, 19-30 days, 20-30 days, 21-30 days, 22-30 days, 23-30 days, 24-30 days, 25-30 days, 26-30 days, 27-30 days, 28-30 days, 29-30 days, 1-4 week, 2-4 weeks, 3-4 weeks, 1-12 months, 2-12 months, 3-12 months, 4-12 months, 5-12 months, 6-12 months, 7-12 months, 8-12 months, 9-12 months, 10-12 months, 11-12 months, or any combination thereof, before the PI3 kinase inhibitor is administered. In some embodiments, the immunogenic vaccine is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the PI3 kinase inhibitor is administered. For example, the immunogenic vaccine can be administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, is administered.

In some embodiments, the immunogenic vaccine is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the PI3 kinase inhibitor is administered. For example, the immunogenic vaccine can be administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, is administered.

In some embodiments, the immunogenic vaccine is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the PI3 kinase inhibitor is administered. For example, the immunogenic vaccine can be administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, is administered.

In some embodiments, the immunogenic vaccine is administered chronologically at the same time as the at least one additional pharmaceutically active agent.

In some embodiments, the immunogenic vaccine is administered chronologically after the PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136. In some embodiments, the PI3 kinase inhibitor is administered from 1-24 hours, 2-24 hours, 3-24 hours, 4-24 hours, 5-24 hours, 6-24 hours, 7-24 hours, 8-24 hours, 9-24 hours, 10-24 hours, 11-24 hours, 12-24 hours, 1-30 days, 2-30 days, 3-30 days, 4-30 days, 5-30 days, 6-30 days, 7-30 days, 8-30 days, 9,-30 days, 10-30 days, 11-30 days, 12-30 days, 13-30 days, 14-30 days, 15-30 days, 16-30 days, 17-30 days, 18-30 days, 19-30 days, 20-30 days, 21-30 days, 22-30 days, 23-30 days, 24-30 days, 25-30 days, 26-30 days, 27-30 days, 28-30 days, 29-30 days, 1-4 week, 2-4 weeks, 3-4 weeks, 1-12 months, 2-12 months, 3-12 months, 4-12 months, 5-12 months, 6-12 months, 7-12 months, 8-12 months, 9-12 months, 10-12 months, 11-12 months, or any combination thereof, before the immunogenic vaccine is administered. In some embodiments the PI3 kinase inhibitor is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered. For example, Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, can be administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered.

In some embodiments the PI3 kinase inhibitor is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered. For example, Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, can be administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered.

In some embodiments the PI3 kinase inhibitor is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered. For example, Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, can be administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered.

In some embodiments, provided herein is a method of treating a condition or disease comprising administering to a patient in need thereof a therapeutically effective amount of a immunogenic vaccine, in combination with a therapeutically effective amount of a PI3 kinase inhibitor. For example, provided herein is a method of treating a condition or disease comprising administering to a patient in need thereof a therapeutically effective amount of a immunogenic vaccine, in combination with a therapeutically effective amount of Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136.

In some embodiments, a immunogenic vaccine is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the immunogenic vaccine/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, or 28 day cycle; and the PI3 kinase inhibitor (e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136) is administered prior to, concomitantly with, or subsequent to administration of the immunogenic vaccine on one or more days (e.g., on day 1 of cycle 1). In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the combination therapy is administered for 1 to 12 or 13 cycles of 28 days (e.g., about 12 months).

In some embodiments, provided herein is a method of treating a condition or disease comprising administering to a patient in need thereof a therapeutically effective amount of a immunogenic vaccine in combination with a therapeutically effective amount of a PI3 kinase inhibitor, e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136, and a secondary active agent, such as a checkpoint inhibitor. In some embodiments, a immunogenic vaccine is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the immunogenic vaccine/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, or 28 day cycle; the PI3 kinase inhibitor (e.g., Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136) is administered prior to, concomitantly with, or subsequent to administration of the immunogenic vaccine on one or more days (e.g., on day 1 of cycle 1), and the secondary agent is administered daily, weekly, or monthly. In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the combination therapy is administered for 1 to 12 or 13 cycles of 28 days (e.g., about 12 months).

In some embodiments, the immunogenic vaccine may be used in combination with inhibitors of the cyclin-dependent kinases, for example with an inhibitor of CDK4 and/or CDK6. An example of such inhibitor that may be used in combination with the instant immunogenic vaccine is palbociclib (IBRANCE) (see, e.g., Clin. Cancer Res.; 2015, 21(13); 2905-10). An example of such inhibitor that may be used in combination with the instant immunogenic vaccine is ribociclib. An example of such inhibitor that may be used in combination with the instant immunogenic vaccine is abemaciclib. An example of such inhibitor that may be used in combination with the instant immunogenic vaccine is seliciclib. An example of such inhibitor that may be used in combination with the instant immunogenic vaccine is dinaciclib. An example of such inhibitor that may be used in combination with the instant immunogenic vaccine is milciclib. An example of such inhibitor that may be used in combination with the instant immunogenic vaccine is roniciclib. An example of such inhibitor that may be used in combination with the instant immunogenic vaccine is atuveciclib. An example of such inhibitor that may be used in combination with the instant immunogenic vaccine is briciclib. An example of such inhibitor that may be used in combination with the instant immunogenic vaccine is riviciclib. An example of such inhibitor that may be used in combination with the instant immunogenic vaccine is seliciclib. An example of such inhibitor that may be used in combination with the instant immunogenic vaccine is trilaciclib. An example of such inhibitor that may be used in combination with the instant immunogenic vaccine is voruciclib. In some examples, the immunogenic vaccines of the disclosure may be used in combination with an inhibitor of CDK4 and/or CDK6 and with an agent that reinforces the cytostatic activity of CDK4/6 inhibitors and/or with an agent that converts reversible cytostasis into irreversible growth arrest or cell death. Exemplary cancer subtypes include NSCLC, melanoma, neuroblastoma, glioblastoma, liposarcoma, and mantle cell lymphoma.

In some embodiments, doses of the cyclin dependent kinase inhibitor, e.g., seliciclib, ribociclib, abemaciclib, or palbociclib employed for human treatment can be in the range of about 0.01 mg/kg to about 100 mg/kg per day (e.g., about 0.1 mg/kg to about 100 mg/kg per day, about 0.1 mg/kg to about 50 mg/kg per day, about 10 mg/kg per day or about 30 mg/kg per day). The desired dose may be conveniently administered in a single dose, or as multiple doses administered at appropriate intervals, for example as two, three, four or more sub-doses per day.

In some embodiments, the dosage of the cyclin dependent kinase inhibitor, e.g., seliciclib, ribociclib, abemaciclib, or palbociclib may be from about 1 ng/kg to about 100 mg/kg. The dosage of the cyclin dependent kinase inhibitor, e.g., seliciclib, ribociclib, abemaciclib, or palbociclib may be at any dosage including, but not limited to, about 1 μg/kg, 25 μg/kg, 50 μg/kg, 75 μg/kg, 100 μμg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

The mode of administration of the immunogenic vaccine and the cyclin dependent kinase inhibitor, e.g., seliciclib, ribociclib, abemaciclib, or palbociclib may be simultaneously or sequentially, wherein the immunogenic vaccine and the at least one additional pharmaceutically active agent are sequentially (or separately) administered. For example, the immunogenic vaccine and the cyclin dependent kinase inhibitor, e.g., seliciclib, ribociclib, abemaciclib, or palbociclib may be provided in a single unit dosage form for being taken together or as separate entities (e.g. in separate containers) to be administered simultaneously or with a certain time difference. This time difference may be between 1 hour and 1 month, e.g., between 1 day and 1 week, e.g., 48 hours and 3 days. In addition, it is possible to administer the immunogenic vaccine via another administration way than the cyclin dependent kinase inhibitor, e.g., seliciclib, ribociclib, abemaciclib, or palbociclib. For example, it may be advantageous to administer either the immunogenic vaccine or the cyclin dependent kinase inhibitor, e.g., seliciclib, ribociclib, abemaciclib, or palbociclib intravenously and the other systemically or orally. For example, the immunogenic vaccine is administered intravenously or subcutaneously and the cyclin dependent kinase inhibitor, e.g., seliciclib, ribociclib, abemaciclib, or palbociclib orally.

In some embodiments, the immunogenic vaccine is administered chronologically before the cyclin dependent kinase inhibitor, e.g., seliciclib, ribociclib, abemaciclib, or palbociclib. In some embodiments, the immunogenic vaccine is administered from 1-24 hours, 2-24 hours, 3-24 hours, 4-24 hours, 5-24 hours, 6-24 hours, 7-24 hours, 8-24 hours, 9-24 hours, 10-24 hours, 11-24 hours, 12-24 hours, 1-30 days, 2-30 days, 3-30 days, 4-30 days, 5-30 days, 6-30 days, 7-30 days, 8-30 days, 9,-30 days, 10-30 days, 11-30 days, 12-30 days, 13-30 days, 14-30 days, 15-30 days, 16-30 days, 17-30 days, 18-30 days, 19-30 days, 20-30 days, 21-30 days, 22-30 days, 23-30 days, 24-30 days, 25-30 days, 26-30 days, 27-30 days, 28-30 days, 29-30 days, 1-4 week, 2-4 weeks, 3-4 weeks, 1-12 months, 2-12 months, 3-12 months, 4-12 months, 5-12 months, 6-12 months, 7-12 months, 8-12 months, 9-12 months, 10-12 months, 11-12 months, or any combination thereof, before the cyclin dependent kinase inhibitor is administered. In some embodiments, the immunogenic vaccine is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the cyclin dependent kinase inhibitor is administered. For example, the immunogenic vaccine can be administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before seliciclib, ribociclib, abemaciclib, or palbociclib is administered.

In some embodiments, the immunogenic vaccine is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the cyclin dependent kinase inhibitor is administered. For example, the immunogenic vaccine can be administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before seliciclib, ribociclib, abemaciclib, or palbociclib is administered.

In some embodiments, the immunogenic vaccine is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the cyclin dependent kinase inhibitor is administered. For example, the immunogenic vaccine can be administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before seliciclib, ribociclib, abemaciclib, or palbociclib is administered.

In some embodiments, the immunogenic vaccine is administered chronologically at the same time as the at least one additional pharmaceutically active agent.

In some embodiments, the immunogenic vaccine is administered chronologically after the cyclin dependent kinase inhibitor, e.g., seliciclib, ribociclib, abemaciclib, or palbociclib. In some embodiments, the cyclin dependent kinase inhibitor is administered from 1-24 hours, 2-24 hours, 3-24 hours, 4-24 hours, 5-24 hours, 6-24 hours, 7-24 hours, 8-24 hours, 9-24 hours, 10-24 hours, 11-24 hours, 12-24 hours, 1-30 days, 2-30 days, 3-30 days, 4-30 days, 5-30 days, 6-30 days, 7-30 days, 8-30 days, 9,-30 days, 10-30 days, 11-30 days, 12-30 days, 13-30 days, 14-30 days, 15-30 days, 16-30 days, 17-30 days, 18-30 days, 19-30 days, 20-30 days, 21-30 days, 22-30 days, 23-30 days, 24-30 days, 25-30 days, 26-30 days, 27-30 days, 28-30 days, 29-30 days, 1-4 week, 2-4 weeks, 3-4 weeks, 1-12 months, 2-12 months, 3-12 months, 4-12 months, 5-12 months, 6-12 months, 7-12 months, 8-12 months, 9-12 months, 10-12 months, 11-12 months, or any combination thereof, before the immunogenic vaccine is administered. In some embodiments the cyclin dependent kinase inhibitor is administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered. For example, seliciclib, ribociclib, abemaciclib, or palbociclib can be administered at least 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered.

In some embodiments the cyclin dependent kinase inhibitor is administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered. For example, seliciclib, ribociclib, abemaciclib, or palbociclib can be administered at most 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered.

In some embodiments the cyclin dependent kinase inhibitor is administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, three weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered. For example, seliciclib, ribociclib, abemaciclib, or palbociclib can be administered about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9, days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 week, 2 weeks, 3 weeks, 4 weeks, 1 month, 2 months, 3 months, 4 months, 5 months, 6 months, 7 months, 8 months, 9 months, 10 months, 11 months, 12 months, or any combination thereof, before the immunogenic vaccine is administered.

In some embodiments, provided herein is a method of treating a condition or disease comprising administering to a patient in need thereof a therapeutically effective amount of a immunogenic vaccine, in combination with a therapeutically effective amount of a cyclin dependent kinase inhibitor. For example, provided herein is a method of treating a condition or disease comprising administering to a patient in need thereof a therapeutically effective amount of a immunogenic vaccine, in combination with a therapeutically effective amount of seliciclib, ribociclib, abemaciclib, or palbociclib.

In some embodiments, a immunogenic vaccine is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the immunogenic vaccine/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, or 28 day cycle; and the cyclin dependent kinase inhibitor (e.g., seliciclib, ribociclib, abemaciclib, or palbociclib) is administered prior to, concomitantly with, or subsequent to administration of the immunogenic vaccine on one or more days (e.g., on day 1 of cycle 1). In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the combination therapy is administered for 1 to 12 or 13 cycles of 28 days (e.g., about 12 months).

In some embodiments, provided herein is a method of treating a condition or disease comprising administering to a patient in need thereof a therapeutically effective amount of a immunogenic vaccine in combination with a therapeutically effective amount of a cyclin dependent kinase inhibitor, e.g., seliciclib, ribociclib, abemaciclib, or palbociclib, and a secondary active agent, such as a checkpoint inhibitor. In some embodiments, a immunogenic vaccine is administered once, twice, or thrice daily for 2, 3, 4, 5, 6, 7, 8, 9, 10, 11, 12, 13, 14, 15, 16, 17, 18, 19, 20, 21, 22, 23, 24, 25, 26, 27, 28, 29, or 30, consecutive days followed by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of rest (e.g., no administration of the immunogenic vaccine/discontinuation of treatment) in a 1, 2, 3, 4, 5, 6, 7, 8, 9, 10, 11, 12, 13, 14, 15, 16, 17, 18, 19, 20, 21, 22, 23, 24, 25, 26, 27, or 28 day cycle; the cyclin dependent kinase inhibitor (e.g., seliciclib, ribociclib, abemaciclib, or palbociclib) is administered prior to, concomitantly with, or subsequent to administration of the immunogenic vaccine on one or more days (e.g., on day 1 of cycle 1), and the secondary agent is administered daily, weekly, or monthly. In some embodiments, the combination therapy is administered for 1, 2, 3, 4, 5, 6, 7, 8, 9, 10, 12, or 13 cycles of 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the combination therapy is administered for 1 to 12 or 13 cycles of 28 days (e.g., about 12 months).

In certain embodiments, an additional therapeutic agent comprises a second immunotherapeutic agent. In some embodiments, the additional immunotherapeutic agent includes, but is not limited to, a colony stimulating factor, an interleukin, an antibody that blocks immunosuppressive functions (e.g., an anti-CTLA-4 antibody, anti-CD28 antibody, anti-CD3 antibody, anti-PD-1 antibody, anti-PD-L1 antibody, anti-TIGIT antibody), an antibody that enhances immune cell functions (e.g., an anti-GITR antibody, an anti-OX-40 antibody, an anti-CD40 antibody, or an anti-4-1BB antibody), a toll-like receptor (e.g., TLR4, TLR7, TLR9), a soluble ligand (e.g., GITRL, GITRL-Fc, OX-40L, OX-40L-Fc, CD40L, CD40L-Fc, 4-1BB ligand, or 4-1BB ligand-Fc), or a member of the B7 family (e.g., CD80, CD86). In some embodiments, the additional immunotherapeutic agent targets CTLA-4, CD28, CD3, PD-1, PD-L1, TIGIT, GITR, OX-40, CD-40, or 4-1BB.

In some embodiments, the additional therapeutic agent is an immune checkpoint inhibitor. In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAG3 antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-PD-1 antibody selected from the group consisting of: nivolumab (OPDIVO), pembrolizumab (KEYTRUDA), pidilzumab, MEDI0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MED14736), and avelumab (MSB0010718C). In some embodiments, the additional therapeutic agent is an anti-CTLA-4 antibody selected from the group consisting of: ipilimumab (YERVOY) and tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566.

In some embodiments, the neoantigen therapeutic can be administered in combination with a biologic molecule selected from the group consisting of: adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF-β, TNF-α, VEGF, P1GF, gamma-IFN, IL-1, IL-2, IL-3, IL-4, IL-5, IL-6, IL-7, IL-12, IL-15, and IL-18.

In some embodiments, treatment with a neoantigen therapeutic described herein can be accompanied by surgical removal of tumors, removal of cancer cells, or any other surgical therapy deemed necessary by a treating physician.

In certain embodiments, treatment involves the administration of a neoantigen therapeutic described herein in combination with radiation therapy. Treatment with an agent can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously.

It will be appreciated that the combination of a neoantigen therapeutic described herein and at least one additional therapeutic agent can be administered in any order or concurrently. In some embodiments, the agent will be administered to patients that have previously undergone treatment with a second therapeutic agent. In certain other embodiments, the neoantigen therapeutic and a second therapeutic agent will be administered substantially simultaneously or concurrently. For example, a subject can be given an agent while undergoing a course of treatment with a second therapeutic agent (e.g., chemotherapy). In certain embodiments, a neoantigen therapeutic will be administered within 1 year of the treatment with a second therapeutic agent. It will further be appreciated that the two (or more) agents or treatments can be administered to the subject within a matter of hours or minutes (i.e., substantially simultaneously).

For the treatment of a disease, the appropriate dosage of a neoantigen therapeutic described herein depends on the type of disease to be treated, the severity and course of the disease, the responsiveness of the disease, whether the agent is administered for therapeutic or preventative purposes, previous therapy, the patient's clinical history, and so on, all at the discretion of the treating physician. The neoantigen therapeutic can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates.

In some embodiments, a neoantigen therapeutic can be administered at an initial higher “loading” dose, followed by one or more lower doses. In some embodiments, the frequency of administration can also change. In some embodiments, a dosing regimen can comprise administering an initial dose, followed by additional doses (or “maintenance” doses) once a week, once every two weeks, once every three weeks, or once every month. For example, a dosing regimen can comprise administering an initial loading dose, followed by a weekly maintenance dose of, for example, one-half of the initial dose. Or a dosing regimen can comprise administering an initial loading dose, followed by maintenance doses of, for example one-half of the initial dose every other week. Or a dosing regimen can comprise administering three initial doses for 3 weeks, followed by maintenance doses of, for example, the same amount every other week.

As is known to those of skill in the art, administration of any therapeutic agent can lead to side effects and/or toxicities. In some cases, the side effects and/or toxicities are so severe as to preclude administration of the particular agent at a therapeutically effective dose. In some cases, therapy must be discontinued, and other agents can be tried. However, many agents in the same therapeutic class display similar side effects and/or toxicities, meaning that the patient either has to stop therapy, or if possible, suffer from the unpleasant side effects associated with the therapeutic agent.

In some embodiments, the dosing schedule can be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

The present disclosure provides methods of administering to a subject a neoantigen therapeutic described herein comprising using an intermittent dosing strategy for administering one or more agents, which can reduce side effects and/or toxicities associated with administration of an agent, chemotherapeutic agent, etc. In some embodiments, a method for treating cancer in a human subject comprises administering to the subject a therapeutically effective dose of a neoantigen therapeutic in combination with a therapeutically effective dose of a chemotherapeutic agent, wherein one or both of the agents are administered according to an intermittent dosing strategy. In some embodiments, a method for treating cancer in a human subject comprises administering to the subject a therapeutically effective dose of a neoantigen therapeutic in combination with a therapeutically effective dose of a second immunotherapeutic agent, wherein one or both of the agents are administered according to an intermittent dosing strategy. In some embodiments, the intermittent dosing strategy comprises administering an initial dose of a neoantigen therapeutic to the subject, and administering subsequent doses of the agent about once every 2 weeks. In some embodiments, the intermittent dosing strategy comprises administering an initial dose of a neoantigen therapeutic to the subject, and administering subsequent doses of the agent about once every 3 weeks. In some embodiments, the intermittent dosing strategy comprises administering an initial dose of a neoantigen therapeutic to the subject, and administering subsequent doses of the agent about once every 4 weeks. In some embodiments, the agent is administered using an intermittent dosing strategy and the additional therapeutic agent is administered weekly.

The present disclosure provides compositions comprising the neoantigen therapeutic described herein. The present disclosure also provides pharmaceutical compositions comprising a neoantigen therapeutic described herein and a pharmaceutically acceptable vehicle. In some embodiments, the pharmaceutical compositions find use in immunotherapy. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a neoantigen therapeutic of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition. Exemplary formulations are listed in WO 2015/095811.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22st Edition, 2012, Pharmaceutical Press, London). In some embodiments, the vehicle is 5% dextrose in water.

The pharmaceutical compositions described herein can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intra-arterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories.

The neoantigenic peptides described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22st Edition, 2012, Pharmaceutical Press, London.

In certain embodiments, pharmaceutical formulations include a neoantigen therapeutic described herein complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the neoantigenic peptides described herein can be produced. Suitable examples of sustained-release preparations include semi-permeable matrices of solid hydrophobic polymers containing an agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The present disclosure provides methods of treatment comprising an immunogenic vaccine. Methods of treatment for a disease (such as cancer or a viral infection) are provided. A method can comprise administering to a subject an effective amount of a composition comprising an immunogenic antigen. In some embodiments, the antigen comprises a viral antigen. In some embodiments, the antigen comprises a tumor antigen.

Non-limiting examples of vaccines that can be prepared include a peptide-based vaccine, a nucleic acid-based vaccine, an antibody based vaccine, a T cell based vaccine, and an antigen-presenting cell based vaccine.

Vaccine compositions can be formulated using one or more physiologically acceptable carriers including excipients and auxiliaries which facilitate processing of the active agents into preparations which can be used pharmaceutically. Proper formulation can be dependent upon the route of administration chosen. Any of the well-known techniques, carriers, and excipients can be used as suitable and as understood in the art.

In some cases, the vaccine composition is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al., J. Clin. Invest. 95:341, 1995), naked cDNA or peptides, encapsulated e.g., in poly(DL-lactide-co-glycolide) (“PLG”) microspheres (see, e.g., Eldridge, et al., Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196:17-32, 1996). Sometimes, a vaccine is formulated as a peptide-based vaccine, or nucleic acid based vaccine in which the nucleic acid encodes the polypeptides. Sometimes, a vaccine is formulated as an antibody based vaccine. Sometimes, a vaccine is formulated as a cell based vaccine.

The amino acid sequence of an identified disease-specific immunogenic neoantigen peptide can be used develop a pharmaceutically acceptable composition. The source of antigen can be, but is not limited to, natural or synthetic proteins, including glycoproteins, peptides, and superantigens; antibody/antigen complexes; lipoproteins; RNA or a translation product thereof, and DNA or a polypeptide encoded by the DNA. The source of antigen may also comprise non-transformed, transformed, transfected, or transduced cells or cell lines. Cells may be transformed, transfected, or transduced using any of a variety of expression or retroviral vectors known to those of ordinary skill in the art that may be employed to express recombinant antigens. Expression may also be achieved in any appropriate host cell that has been transformed, transfected, or transduced with an expression or retroviral vector containing a DNA molecule encoding recombinant antigen(s). Any number of transfection, transformation, and transduction protocols known to those in the art may be used. Recombinant vaccinia vectors and cells infected with the vaccinia vector, may be used as a source of antigen.

A composition can comprise a synthetic disease-specific immunogenic neoantigen peptide. A composition can comprise two or more disease-specific immunogenic neoantigen peptides. A composition may comprise a precursor to a disease-specific immunogenic peptide (such as a protein, peptide, DNA and RNA). A precursor to a disease-specific immunogenic peptide can generate or be generated to the identified disease-specific immunogenic neoantigen peptide. In some embodiments, a therapeutic composition comprises a precursor of an immunogenic peptide. The precursor to a disease-specific immunogenic peptide can be a pro-drug. In some embodiments, the composition comprising a disease-specific immunogenic neoantigen peptide may further comprise an adjuvant. For example, the neoantigen peptide can be utilized as a vaccine. In some embodiments, an immunogenic vaccine may comprise a pharmaceutically acceptable immunogenic neoantigen peptide. In some embodiments, an immunogenic vaccine may comprise a pharmaceutically acceptable precursor to an immunogenic neoantigen peptide (such as a protein, peptide, DNA and RNA). In some embodiments, a method of treatment comprises administering to a subject an effective amount of an antibody specifically recognizing an immunogenic neoantigen peptide. In some embodiments, a method of treatment comprises administering to a subject an effective amount of a soluble TCR or TCR analog specifically recognizing an immunogenic neoantigen peptide.

The methods described herein are particularly useful in the personalized medicine context, where immunogenic neoantigen peptides are used to develop therapeutics (such as vaccines or therapeutic antibodies) for the same individual. Thus, a method of treating a disease in a subject can comprise identifying an immunogenic neoantigen peptide in a subject according to the methods described herein; and synthesizing the peptide (or a precursor thereof); and administering the peptide or an antibody specifically recognizing the peptide to the subject. In some embodiments, an expression pattern of an immunogenic neoantigen can serve as the essential basis for the generation of patient specific vaccines. In some embodiments, an expression pattern of an immunogenic neoantigen can serve as the essential basis for the generation of a vaccine for a group of patients with a particular disease. Thus, particular diseases, e.g., particular types of tumors, can be selectively treated in a patient group.

In some embodiments, the peptides described herein are structurally normal antigens that can be recognized by autologous anti-disease T cells in a large patient group. In some embodiments, an antigen-expression pattern of a group of diseased subjects whose disease expresses structurally normal neoantigens is determined.

In some embodiments, the peptides described herein comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, the peptides described herein comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof.

There are a variety of ways in which to produce immunogenic neoantigens. Proteins or peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. In general, such disease specific neoantigens may be produced either in vitro or in vivo. Immunogenic neoantigens may be produced in vitro as peptides or polypeptides, which may then be formulated into a personalized vaccine or immunogenic composition and administered to a subject. In vitro production of immunogenic neoantigens can comprise peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, immunogenic neoantigens can be produced in vivo by introducing molecules (e.g., DNA, RNA, and viral expression systems) that encode an immunogenic neoantigen into a subject, whereupon the encoded immunogenic neoantigens are expressed. In some embodiments, a polynucleotide encoding an immunogenic neoantigen peptide can be used to produce the neoantigen peptide in vitro.

In some embodiments, a polynucleotide comprises a sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a polynucleotide encoding an immunogenic neoantigen.

The polynucleotide may be, e.g., DNA, cDNA, PNA, CNA, RNA, single- and/or double-stranded, native or stabilized forms of polynucleotides, or combinations thereof. A nucleic acid sequence encoding an immunogenic neoantigen peptide may or may not contain introns so long as the nucliec acid sequence codes for the peptide. In some embodiments in vitro translation is used to produce the peptide.

Expression vectors comprising sequences encoding the neoantigen, as well as host cells containing the expression vectors, are also contemplated. Expression vectors suitable for use in the present disclosure can comprise at least one expression control element operationally linked to the nucleic acid sequence. The expression control elements are inserted in the vector to control and regulate the expression of the nucleic acid sequence. Examples of expression control elements are well known in the art and include, for example, the lac system, operator and promoter regions of phage lambda, yeast promoters and promoters derived from polyoma, adenovirus, retrovirus or SV40. Additional operational elements include, but are not limited to, leader sequences, termination codons, polyadenylation signals and any other sequences necessary or preferred for the appropriate transcription and subsequent translation of the nucleic acid sequence in the host system. It will be understood by one skilled in the art the correct combination of expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers.

The neoantigen peptides may be provided in the form of RNA or cDNA molecules encoding the desired neoantigen peptides. One or more neoantigen peptides of the present disclosure may be encoded by a single expression vector. Generally, the DNA is inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques. Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

In embodiments, a DNA sequence encoding a polypeptide of interest can be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems can also be employed. Appropriate cloning and expression vectors for use with bacterial, fungal, yeast, and mammalian cellular hosts are well known in the art. Various mammalian or insect cell culture systems can be employed to express recombinant protein. Exemplary mammalian host cell lines include, but are not limited to COS-7, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

A vaccine can comprise an entity that binds a polypeptide sequence described herein. The entity can be an antibody. Antibody-based vaccine can be formulated using any of the well-known techniques, carriers, and excipients as suitable and as understood in the art. In some embodiments, the peptides described herein can be used for making neoantigen specific therapeutics such as antibody therapeutics. For example, neoantigens can be used to raise and/or identify antibodies specifically recognizing the neoantigens. These antibodies can be used as therapeutics. The antibody can be a natural antibody, a chimeric antibody, a humanized antibody, or can be an antibody fragment. The antibody may recognize one or more of the polypeptides described herein. In some embodiments, the antibody can recognize a polypeptide that has a sequence with at most 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a polypeptide described herein. In some embodiments, the antibody can recognize a polypeptide that has a sequence with at least 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polypeptide described herein. In some embodiments, the antibody can recognize a polypeptide sequence that is at least 30%, 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a length of a polypeptide described herein. In some embodiments, the antibody can recognize a polypeptide sequence that is at most 30%, 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a length of a polypeptide described herein.

The present disclosure also contemplates the use of nucleic acid molecules as vehicles for delivering neoantigen peptides/polypeptides to the subject in need thereof, in vivo, in the form of, e.g., DNA/RNA vaccines.

In some embodiments, the vaccine is a nucleic acid vaccine. In some embodiments, neoantigens can be administered to a subject by use of a plasmid. Plasmids may be introduced into animal tissues by a number of different methods, e.g., injection or aerosol instillation of naked DNA on mucosal surfaces, such as the nasal and lung mucosa. In some embodiments, physical delivery, such as with a “gene-gun” may be used. The exact choice of expression vectors can depend upon the peptide/polypeptides to be expressed, and is well within the skill of the ordinary artisan.

In some embodiments, the nucleic acid encodes an immunogenic peptide or peptide precursor. In some embodiments, the nucleic acid vaccine comprises sequences flanking the sequence coding the immunogenic peptide or peptide precursor. In some embodiments, the nucleic acid vaccine comprises more than one immunogenic epitope. In some embodiments, the nucleic acid vaccine is a DNA-based vaccine. In some embodiments, the nucleic acid vaccine is a RNA-based vaccine. In some embodiments, the RNA-based vaccine comprises mRNA. In some embodiments, the RNA-based vaccine comprises naked mRNA. In some embodiments, the RNA-based vaccine comprises modified mRNA (e.g., mRNA protected from degradation using protamine. mRNA containing modified 5′ CAP structure or mRNA containing modified nucleotides). In some embodiments, the RNA-based vaccine comprises single-stranded mRNA.

The polynucleotide may be substantially pure, or contained in a suitable vector or delivery system. Suitable vectors and delivery systems include viral, such as systems based on adenovirus, vaccinia virus, retroviruses, herpes virus, adeno-associated virus or hybrids containing elements of more than one virus. Non-viral delivery systems include cationic lipids and cationic polymers (e.g., cationic liposomes).

One or more neoantigen peptides can be encoded and expressed in vivo using a viral based system. Viral vectors may be used as recombinant vectors in the present disclosure, wherein a portion of the viral genome is deleted to introduce new genes without destroying infectivity of the virus. The viral vector of the present disclosure is a nonpathogenic virus. In some embodiments the viral vector has a tropism for a specific cell type in the mammal. In another embodiment, the viral vector of the present disclosure is able to infect professional antigen presenting cells such as dendritic cells and macrophages. In yet another embodiment of the present disclosure, the viral vector is able to infect any cell in the mammal. The viral vector may also infect tumor cells. Viral vectors used in the present disclosure include but is not limited to Poxvirus such as vaccinia virus, avipox virus, fowlpox virus and a highly attenuated vaccinia virus (Ankara or MVA), retrovirus, adenovirus, baculovirus and the like.

A vaccine can be delivered via a variety of routes. Delivery routes can include oral (including buccal and sub-lingual), rectal, nasal, topical, transdermal patch, pulmonary, vaginal, suppository, or parenteral (including intramuscular, intra-arterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999). The vaccine described herein can be administered to muscle, or can be administered via intradermal or subcutaneous injections, or transdermally, such as by iontophoresis. Epidermal administration of the vaccine can be employed.

In some instances, the vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can include material for a single immunization, or may include material for multiple immunizations (i.e. a ‘multidose’ kit). The inclusion of a preservative is preferred in multidose arrangements. As an alternative (or in addition) to including a preservative in multidose compositions, the compositions can be contained in a container having an aseptic adaptor for removal of material.

The vaccine can be administered in a dosage volume of about 0.5 mL, although a half dose (i.e. about 0.25 mL) can be administered to children. Sometimes the vaccine can be administered in a higher dose e.g. about 1 ml.

The vaccine can be administered as a 1, 2, 3, 4, 5, 6, 7, 8, 9, 10, or more dose-course regimen. Sometimes, the vaccine is administered as a 1, 2, 3, or 4 dose-course regimen. Sometimes the vaccine is administered as a 1 dose-course regimen. Sometimes the vaccine is administered as a 2 dose-course regimen.

The administration of the first dose and second dose can be separated by about 0 day, 1 day, 2 days, 5 days, 7 days, 14 days, 21 days, 30 days, 2 months, 4 months, 6 months, 9 months, 1 year, 1.5 years, 2 years, 3 years, 4 years, or more.

The vaccine described herein can be administered every 1, 2, 3, 4, 5, 6, 7, 8, 9, 10, or more years. Sometimes, the vaccine described herein is administered every 2, 3, 4, 5, 6, 7, or more years. Sometimes, the vaccine described herein is administered every 4, 5, 6, 7, or more years. Sometimes, the vaccine described herein is administered once.

The dosage examples are not limiting and are only used to exemplify particular dosing regiments for administering a vaccine described herein. The effective amount for use in humans can be determined from animal models. For example, a dose for humans can be formulated to achieve circulating, liver, topical and/or gastrointestinal concentrations that have been found to be effective in animals. Based on animal data, and other types of similar data, those skilled in the art can determine the effective amounts of a vaccine composition appropriate for humans.

The effective amount when referring to an agent or combination of agents will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

In some aspects, the vaccine and kit described herein can be stored at between 2° C. and 8° C. In some instances, the vaccine is not stored frozen. In some instances, the vaccine is stored in temperatures of such as at −20° C. or −80° C. In some instances, the vaccine is stored away from sunlight.

### Kits

The neoantigen therapeutic described herein can be provided in kit form together with instructions for administration. Typically the kit would include the desired neoantigen therapeutic in a container, in unit dosage form and instructions for administration. Additional therapeutics, for example, cytokines, lymphokines, checkpoint inhibitors, antibodies, can also be included in the kit. Other kit components that can also be desirable include, for example, a sterile syringe, booster dosages, and other desired excipients.

Kits and articles of manufacture are also provided herein for use with one or more methods described herein. The kits can contain one or more neoantigenic polypeptides comprising one or more neoepitopes. The kits can also contain nucleic acids that encode one or more of the peptides or proteins described herein, antibodies that recognize one or more of the peptides described herein, or APC-based cells activated with one or more of the peptides described herein. The kits can further contain adjuvants, reagents, and buffers necessary for the makeup and delivery of the vaccines.

The kits can also include a carrier, package, or container that is compartmentalized to receive one or more containers such as vials, tubes, and the like, each of the container(s) comprising one of the separate elements, such as the peptides and adjuvants, to be used in a method described herein. Suitable containers include, for example, bottles, vials, syringes, and test tubes. The containers can be formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment. A kit typically includes labels listing contents and/or instructions for use, and package inserts with instructions for use. A set of instructions will also typically be included.

The present disclosure will be described in greater detail by way of specific examples. The following examples are offered for illustrative purposes, and are not intended to limit the present disclosure in any manner. Those of skill in the art will readily recognize a variety of non-critical parameters that can be changed or modified to yield alternative embodiments according to the present disclosure. All patents, patent applications, and printed publications listed herein are incorporated herein by reference in their entirety.

### EXAMPLES

These examples are provided for illustrative purposes only and not to limit the scope of the claims provided herein.

### Example 1—Induction of CD4⁺ and CD8⁺ T Cell Responses

In vitro T cell inductions are used to expand neo-antigen specific T cells. Mature professional APCs are prepared for these assays in the following way. Monocytes are enriched from healthy human donor PBMCs using a bead-based kit (Miltenyi). Enriched cells are plated in GM-CSF and IL-4 to induce immature DCs. After 5 days, immature DCs are incubated at 37° C. with pools of peptides for 1 hour before addition of a cytokine maturation cocktail (GM-CSF, IL-1β, IL-4, IL-6, TNFα, PGE1β). The pools of peptides can include multiple mutations, with both shortmers and longmers to expand CD8⁺ and CD4⁺ T cells, respectively. Cells are incubated at 37° C. to mature DCs.

After maturation of DCs, PBMCs (either bulk or enriched for T cells) are added to mature dendritic cells with proliferation cytokines. Cultures are monitored for peptide-specific T cells using a combination of functional assays and/or tetramer staining. Parallel immunogenicity assays with the modified and parent peptides allowed for comparisons of the relative efficiency with which the peptides expanded peptide-specific T cells.

### Example 2—Tetramer Staining Assay

MHC tetramers are purchased or manufactured on-site, and are used to measure peptide-specific T cell expansion in the immunogenicity assays. For the assessment, tetramer is added to 1×10⁵ cells in PBS containing 1% FCS and 0.1% sodium azide (FACS buffer) according to manufacturer's instructions. Cells are incubated in the dark for 20 minutes at room temperature. Antibodies specific for T cell markers, such as CD8, are then added to a final concentration suggested by the manufacturer, and the cells are incubated in the dark at 4° C. for 20 minutes. Cells are washed with cold FACS buffer and resuspended in buffer containing 1% formaldehyde. Cells are acquired on a FACS Calibur (Becton Dickinson) instrument, and are analyzed by use of Cellquest software (Becton Dickinson). For analysis of tetramer positive cells, the lymphocyte gate is taken from the forward and side-scatter plots. Data are reported as the percentage of cells that were CD8⁺/tetramer⁺.

### Example 3—Intracellular Cytokine Staining Assay

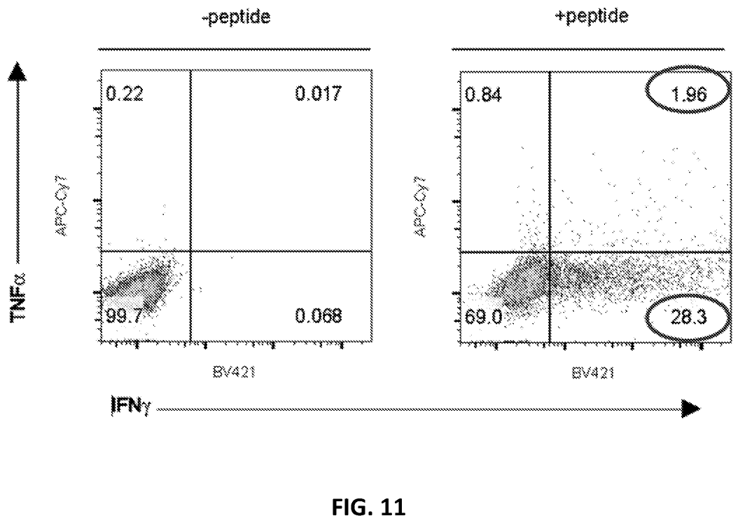

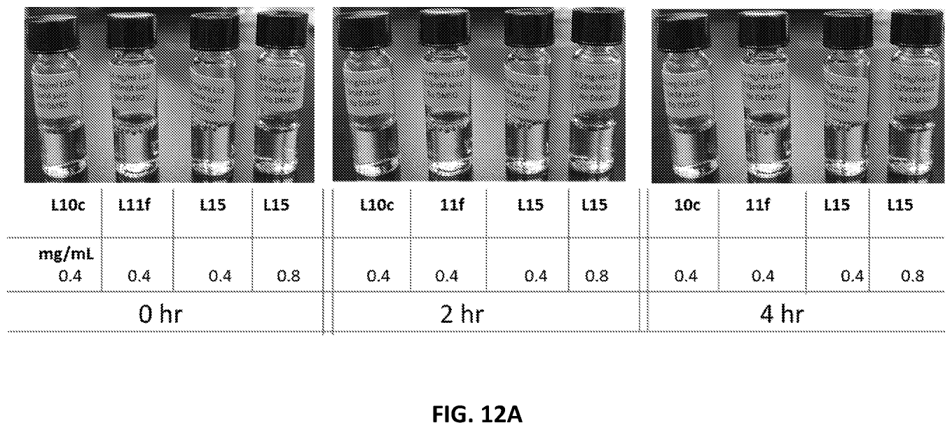

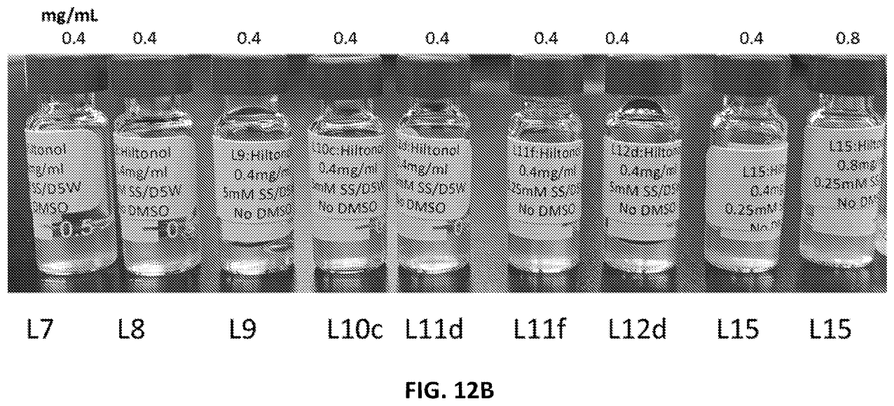

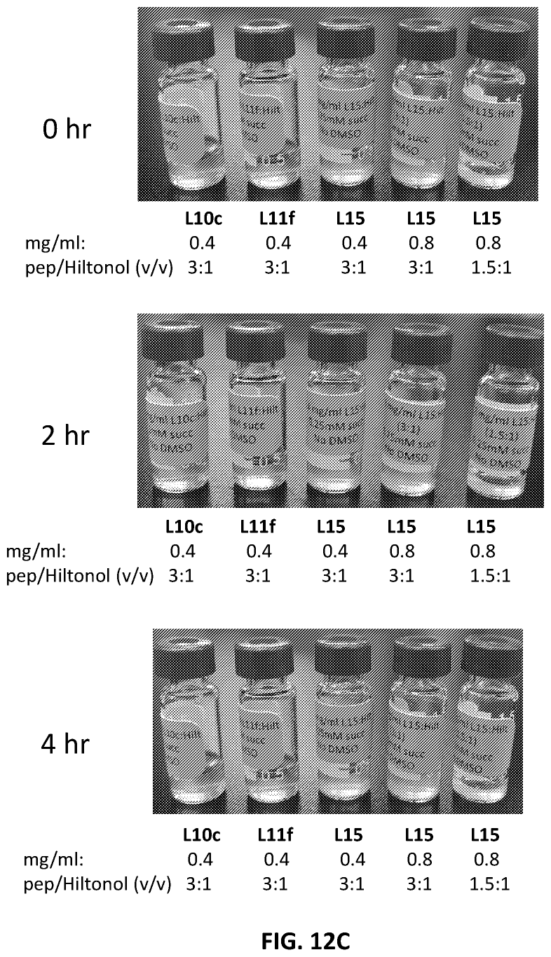

In the absence of well-established tetramer staining to identify antigen-specific T cell populations, antigen-specificity can be estimated using assessment of cytokine production using well-established flow cytometry assays. Briefly, T cells are stimulated with the peptide of interest and compared to a control. After stimulation, production of cytokines by CD4⁺ T cells (e.g., IFNγ and TNFα) are assessed by intracellular staining. These cytokines, especially IFNγ, can be used to identify stimulated cells. FIG. 11 depicts a FACS analysis of antigen-specific induction of IFNγ and TNFα levels of CD4⁺ cells from a healthy HLA-A02:01 donor stimulated with APCs loaded with or without a GATA3 neoORF peptide.

### Example 4—ELISPOT Assay

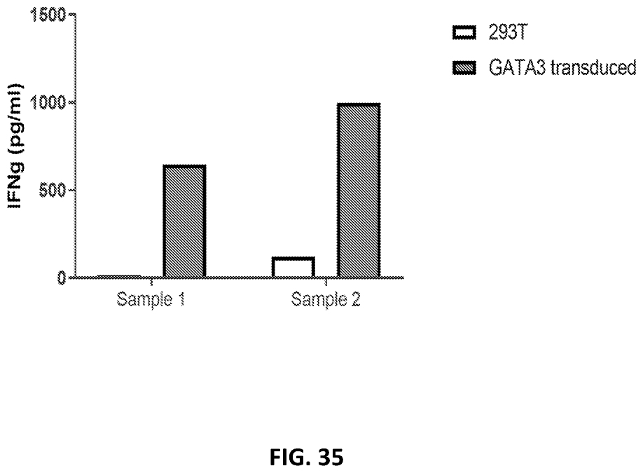

Peptide-specific T cells are functionally enumerated using the ELISPOT assay (BD Biosciences), which measures the release of IFNγ from T cells on a single cell basis. Target cells (T2 or HLA-A0201 transfected C1Rs) were pulsed with 10 μM peptide for 1 hour at 37° C., and washed three times. 1×10⁵ peptide-pulsed targets are co-cultured in the ELISPOT plate wells with varying concentrations of T cells (5×10² to 2×10³) taken from the immunogenicity culture. Plates are developed according to the manufacturer's protocol, and analyzed on an ELISPOT reader (Cellular Technology Ltd.) with accompanying software. Spots corresponding to the number of IFNγ-producing T cells are reported as the absolute number of spots per number of T cells plated. T cells expanded on modified peptides are tested not only for their ability to recognize targets pulsed with the modified peptide, but also for their ability to recognize targets pulsed with the parent peptide. FIG. 35 is a graph showing antigen-specific induction of IFNγ. The IFNγ levels of two samples mock transduced or transduced with a lentiviral expression vector encoding a GATA3 neoORF peptide are shown.

### Example 5—CD107 Staining Assay

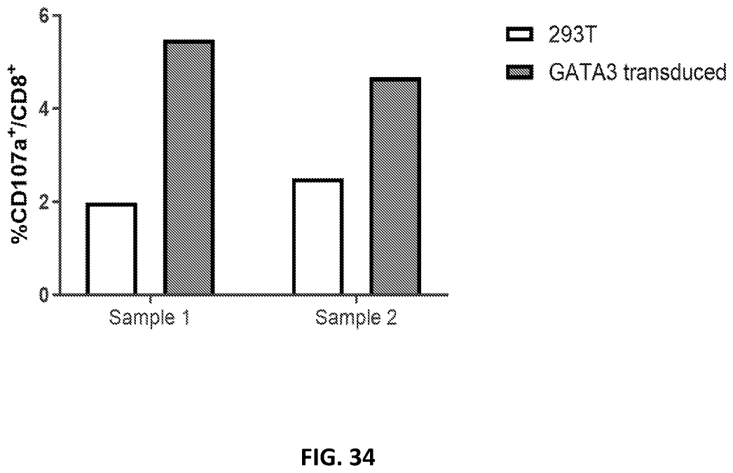

CD107a and b are expressed on the cell surface of CD8⁺ T cells following activation with cognate peptide. The lytic granules of T cells have a lipid bilayer that contains lysosomal-associated membrane glycoproteins (“LAMPs”), which include the molecules CD107a and b. When cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107a and b staining is reported on a single cell basis, the assay is used to functionally enumerate peptide-specific T cells. To perform the assay, peptide is added to HLA-A02:01-transfected cells CIR to a final concentration of 20 μM, the cells were incubated for 1 hour at 37° C., and washed three times. 1×10⁵ of the peptide-pulsed C1R cells were aliquoted into tubes, and antibodies specific for CD107a and b are added to a final concentration suggested by the manufacturer (Becton Dickinson). Antibodies are added prior to the addition of T cells in order to “capture” the CD107 molecules as they transiently appear on the surface during the course of the assay. 1×10⁵ T cells from the immunogenicity culture are added next, and the samples were incubated for 4 hours at 37° C. The T cells are further stained for additional cell surface molecules such as CD8 and acquired on a FACS Calibur instrument (Becton Dickinson). Data is analyzed using the accompanying Cellquest software, and results are reported as the percentage of CD8⁺/CD107a and b⁺ cells. FIG. 34 is a graph showing antigen-specific induction of the cytotoxic marker CD107a. The percent CD107a+ cells of total CD8+ cells of two samples mock transduced or transduced with a lentiviral expression vector encoding a GATA3 neoORF peptide are shown.

### Example 6—Cytotoxicity Assays

Cytotoxic activity is measured using method 1 or method 2. Method 1 entails a chromium release assay. Target T2 cells are labeled for 1 hour at 37° C. with Na⁵¹Cr and washed 5×10³ target T2 cells are then added to varying numbers of T cells from the immunogenicity culture. Chromium release is measured in supernatant harvested after 4 hours of incubation at 37° C. The percentage of specific lysis is calculated as:

Experimental release-spontaneous release/Total release-spontaneous release×100. Equation 10.

In method 2 cytotoxicity activity is measured with the detection of cleaved Caspase 3 in target cells by Flow cytometry. Target cancer cells are engineered to express the mutant peptide along with the proper MHC-I allele. Mock-transduced target cells (i.e. not expressing the mutant peptide) are used as a negative control. The cells are labeled with CFSE to distinguish them from the stimulated PBMCs used as effector cells. The target and effector cells are co-cultured for 6 hours before being harvested. Intracellular staining is performed to detect the cleaved form of Caspase 3 in the CFSE-positive target cancer cells. The percentage of specific lysis is calculated as:

Experimental cleavage of Caspase 3/spontaneous cleavage of Caspase 3(measured in the absence of mutant peptide expression)×100. Equation 11.

The method 2 cytotoxicity assay is provided in materials and methods section of Example 25 herein.

### Example 7—Enhanced CD8⁺ T Cell Responses In Vivo Using Longmers and Shortmers Sequentially

Vaccination with longmer peptides can induce both CD4⁺ and CD8⁺ T cell responses, depending on the processing and presentation of the peptides. Vaccination with minimal shortmer epitopes focuses on generating CD8⁺ T cell responses, but does not require peptide processing before antigen presentation. As such, any cell can present the epitope readily, not just professional antigen-presenting cells (APCs). This may lead to tolerance of T cells that come in contact with healthy cells presenting antigens as part of peripheral tolerance. To circumvent this, initial immunization with longmers allows priming of CD8⁺ T cells only by APCs that can process and present the peptides. Subsequent immunizations boosts the initial CD8⁺ T cell responses.

### In Vivo Immunogenicity Assays

Nineteen 8-12 week old female C57BL/6 mice (Taconic Biosciences) were randomly and prospectively assigned to treatment groups on arrival. Animals were acclimated for three (3) days prior to study commencement. Animals were maintained on LabDiet™ 5053 sterile rodent chow and sterile water provided ad libitum. Animals in Group 1 served as vaccination adjuvant-only controls and were administered polyinosinic:polycytidylic acid (polyL:C) alone at 100 μg in a volume of 0.1 mL administered via subcutaneous injection (s.c.) on day 0, 7, and 14. Animals in Group 2 were administered 50 μg each of six longmer peptides (described below) along with polyL:C at 100 μg s.c. in a volume of 0.1 mL on day 0, 7 and 14. Animals in Group 3 were administered 50 μg each of six longmer peptides (described below) along with polyL:C at 100 μg s.c. in a volume of 0.1 mL on day 0 and molar-matched equivalents of corresponding shortmer peptides (described below) along with polyL:C at 100 μg s.c. in a volume of 0.1 mL on day 7 and 14. Animals were weighed and monitored for general health daily. Animals were euthanized by CO2 overdose at study completion Day 21, if an animal lost >30% of its body weight compared to weight at Day 0; or if an animal was found moribund. At sacrifice, spleens were harvested and processed into single-cell suspensions using standard protocols. Briefly, spleens were mechanical degraded through a 70 μM filter, pelleted, and lysed with ACK lysis buffer (Sigma) before resuspension in cell culture media.

### Peptides

Six previously identified murine neoantigens were used based on their demonstrated ability to induce CD8⁺ T cell responses. For each neoantigen, shortmers (8-11 amino acids) corresponding to the minimal epitope have been defined. Longmers corresponding to 20-27 amino acids surrounding the mutation were used.

### ELISPOT

ELISPOT analysis (Mouse IFNγ ELISPOT Reasy-SET-Go; EBioscience) was performed according to the kit protocol. Briefly, one day prior to day of analysis, 96-well filter plates (0.45 μm pore size hydrophobic PVDF membrane; EMD Millipore) were activated (35% EtOH), washed (PBS) and coated with capture antibody (1:250; 4° C. O/N). On the day of analysis, wells were washed and blocked (media; 2 hours at 37° C.). Approximately 2×10⁵ cells in 100 μL was added to the wells along with 100 μL of 10 mM test peptide pool (shortmers), or PMA/ionomycin positive control antigen, or vehicle. Cells incubated with antigen overnight (16-18 hours) at 37° C. The next day, the cell suspension was discarded, and wells were washed once with PBS, and twice with deionized water. For all wash steps in the remainder of the assay, wells were allowed to soak for 3 minutes at each wash step. Wells were then washed three times with wash buffer (PBS+0.05% Tween-20), and detection antibody (1:250) was added to all wells. Plates were incubated for two hours at room temperature. The detection antibody solution was discarded, and wells were washed three times with wash buffer. Avidin-HRP (1:250) was added to all wells, and plates were incubated for one hour at room temperature. Conjugate solution was discarded, and wells washed three times with wash buffer, then once with PBS. Substrate (3-amino-9-ethyl-carbazole, 0.1 M Acetate buffer, H₂O₂) was added to all wells, and spot development monitored (approximately 10 minutes). Substrate reaction was stopped by washing wells with water, and plates were allowed to air-dry overnight. The plates were analyzed on an ELISPOT reader (Cellular Technology Ltd.) with accompanying software. Spots corresponding to the number of IFNγ-producing T cells are reported as the absolute number of spots per number of T cells plated.

### Example 8—Detection of GATA3 neoORF Peptides by Mass Spectrometry

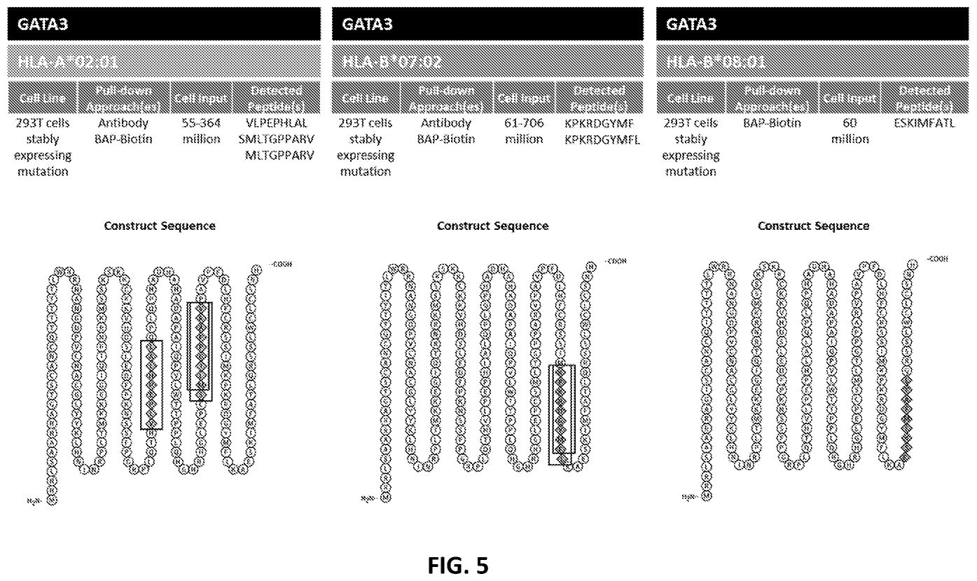

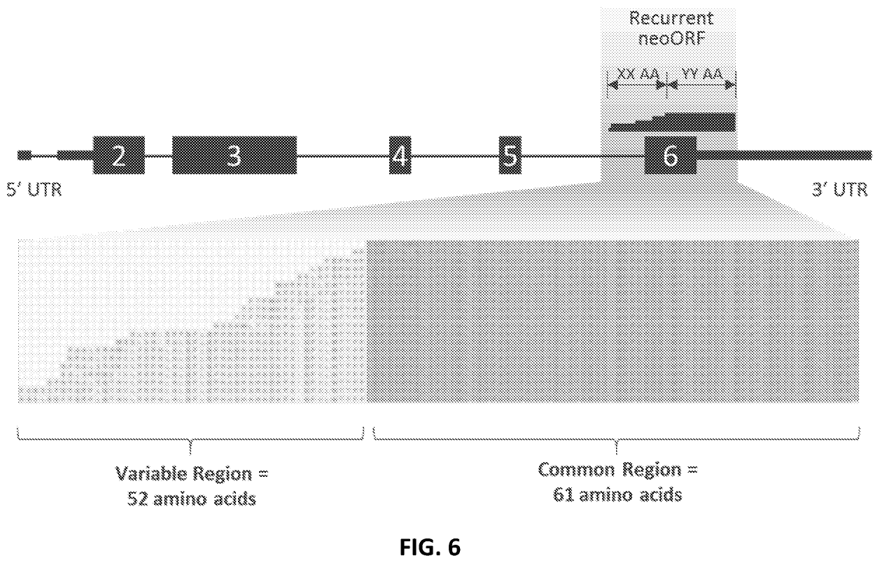

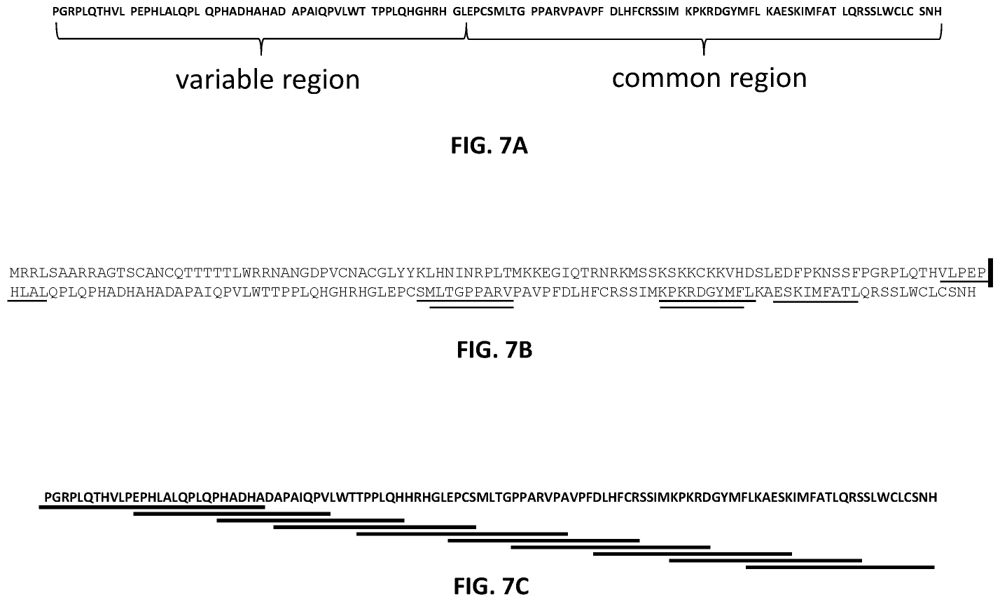

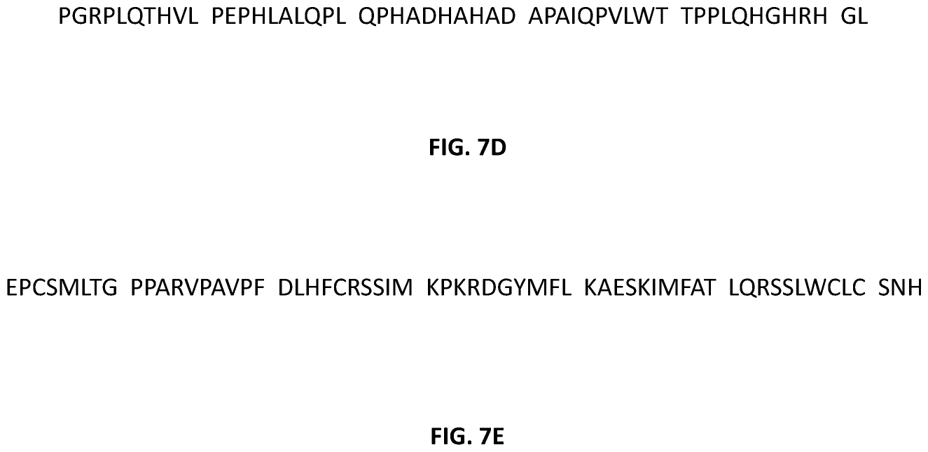

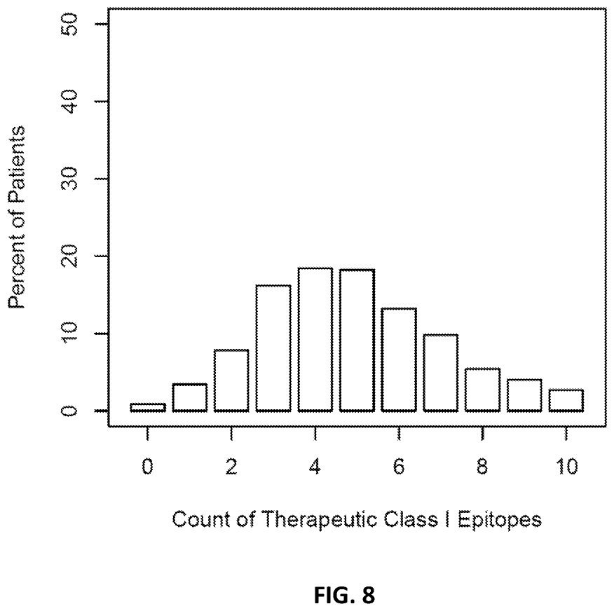

293T cells were transduced with a lentiviral vector encoding various regions of peptides encoded by the GATA3 neoORF. 50-700 million of the transduced cells expressing peptides encoded by the GATA3 neoORF sequence were cultured and peptides were eluted from HLA-peptide complexes using an acid wash. Eluted peptides were then analyzed by MS/MS. For 293T cells expressing an HLA-A02:01 protein, the peptides VLPEPHLAL, SMLTGPPARV and MLTGPPARV were detected by mass spectrometry (FIG. 5). For 293T cells expressing an HLA-B07:02 protein, the peptides KPKRDGYMF and KPKRDGYMFL were detected by mass spectrometry (FIG. 5). For 293T cells expressing an HLA-B08:01 protein, the peptide ESKIMFATL was detected by mass spectrometry (FIG. 5).

### Example 9—GATA3 neoORF Produces Strong Epitopes on Multiple Alleles

Multiple peptides containing the neoepitopes in Table 4 below were expressed or loaded onto antigen presenting cells (APCs). Mass spectrometry was then performed and the affinity of the neoepitopes for the indicated HLA alleles and stability of the neoepitopes with the HLA alleles was determined.

TABLE 4
lists exemplary GATA3 neoORF
produced epitopes on multiple alleles

| | | Common | Observed | Observed |
|---|---|---|---|---|
| | | (C) or | MHC | MHC |
| | Neo- | variable | affinity | stability |
| Allele | epitope | (V) region | (nM) | (hr) |
| A02.01 | AIQPVLWTT | V | 8 | 1.5 |
| | MLTGPPARV | C | 11 | 5.8 |
| | SMLTGPPARV | C | 14 | 21.7 |
| | VLPEPHLAL | V | 16 | 1.1 |
| | TLQRSSLWCL | C | 118 | 0.5 |
| | YMFLKAESKI | C | 141 | 0.6 |
| | ALQPLQPHA | V | 604 | 1.5 |
| A03:01 | KIMFATLQR | C | 3 | 5.6 |
| | VLWTTPPLQH | V | 16 | 0.3 |
| | YMFLKAESK | C | 80 | 0.3 |
| A11:01 | KIMFATLQR | C | 23 | 8.9 |
| | VLWTTPPLQH | V | 4539 | 0 |
| | YMFLKAESK | C | 1729 | 0 |
| A24:02 | MFLKAESKI | C | 332 | 0.2 |
| | YMFLKAESKI | C | 6,995 | 1.2 |
| B07:02 | FATLQRSSL | C | 14 | 0.7 |
| | EPHLALQPL | V | 17 | 7.2 |
| | KPKRDGYMF | C | 28 | 8.6 |
| | KPKRDGYMFL | C | 98 | 3.3 |
| | QPVLWTTPPL | V | 109 | 1.4 |
| | GPPARVPAV | C | 221 | 1.6 |
| | MFATLQRSSL | V | 267 | 0 |
| B08:01 | EPHLALQPL | V | 12 | 0 |
| | ESKIMFATL | C | 18 | 1.3 |
| | FLKAESKIM | C | 22 | 1.2 |
| | FATLQRSSL | C | 27 | 0 |
| | YMFLKAESKI | C | 32 | 0.4 |
| | IMKPKRDGYM | C | 33 | 0.4 |
| | MFATLQRSSL | C | 53 | 0 |
| | FLKAESKIMF | C | 82 | 0 |
| | LHFCRSSIM | C | 119 | 0 |

### Example 10—Multiple Neoepitopes Elicit CD8⁺ T Cell Responses

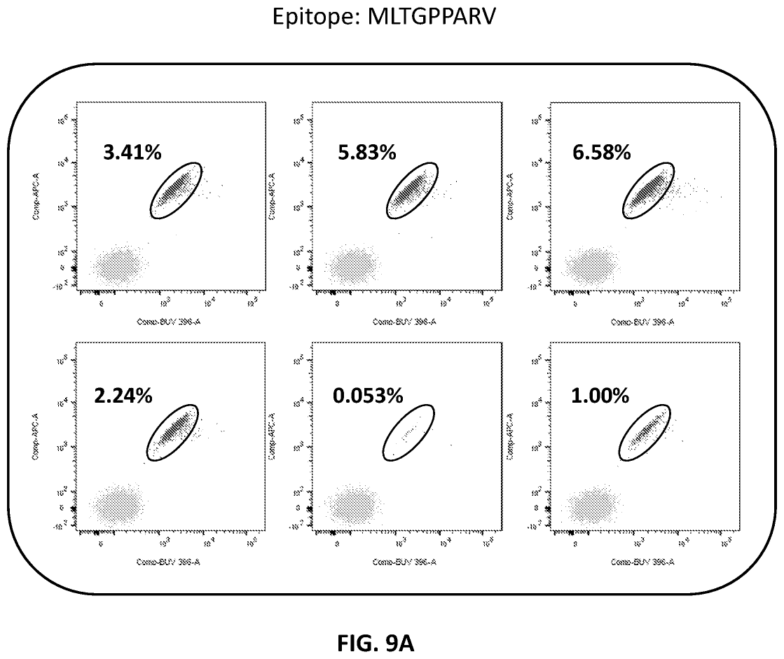

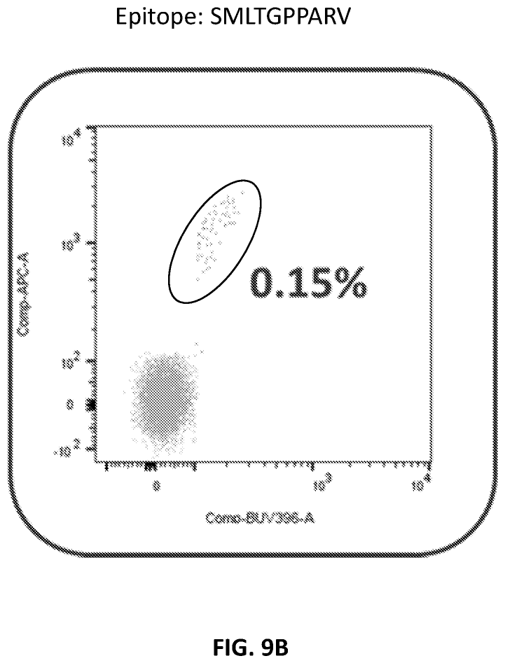

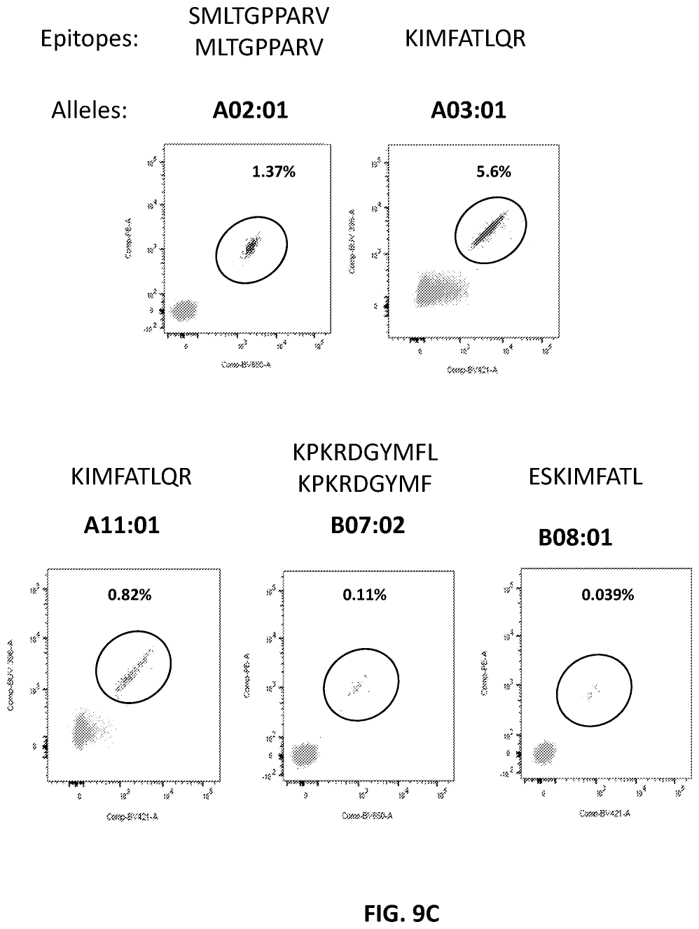

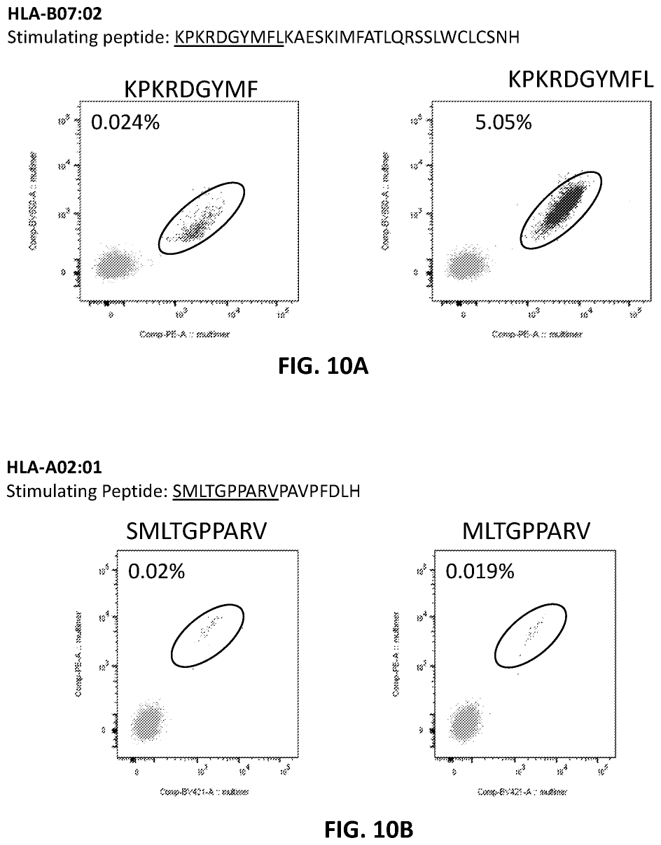

PBMC samples from a human donor were used to perform antigen specific T cell induction. CD8⁺ T cell inductions were analyzed after manufacturing T cells. Cell samples can be taken out at different time points for analysis. pMIHC multimers were used to monitor the fraction of antigen specific CD8⁺ T cells in the induction cultures. FIGS. 9A-9C and 10A-10B depict exemplary results showing the fraction of antigen specific CD8⁺ memory T cells induced with and SMLTGPPARV and MLTGPPARV, respectively. FIG. 9A depicts an exemplary result of a T cell response assay using PBMCs from 6 different healthy donors showing the fraction of antigen specific CD8⁺ T cells that responded to MLTGPPARV peptide analyzed by flow cytometry after stimulation or induction. An increase in the fraction of antigen specific T cells was observed. FIG. 9B depicts an exemplary result of a T cell response assay using PBMCs from a healthy donor showing fraction of antigen specific CD8⁺ T cells that responded to SMLTGPPARV peptide analyzed by flow cytometry after stimulation or induction. An increase in the fraction of antigen specific T cells was observed. Of the five healthy donors tested, 4 showed an increase in the fraction of antigen specific CD8⁺ T cells that responded to MLTGPPARV peptide. A T cell response assay using PBMCs from 3 different healthy donors showed an increase in the fraction of antigen specific CD8⁺ T cells that responded to VLPEPHLAL peptide analyzed by flow cytometry after stimulation or induction in one of the three donors. FIG. 9C depicts an exemplary result of a T cell response assay using PBMCs from HLA-A02:01, HLA-A03:01 HLA-A11:01, HLA-B07:02 and HLA-B08:01 healthy donors showing fraction of antigen specific CD8⁺ T cells that responded to SMLTGPPARV, MLTGPPARV, KIMFATLQR, KPKRDGYMFL KPKRDGYMF or ESKIMFATL peptide analyzed by flow cytometry after stimulation or induction. FIG. 10A depicts an exemplary result of a T cell response assay using PBMCs from an HLA-B07:02 healthy donor showing fraction of antigen specific CD8⁺ T cells that responded to stimulating peptide KPKRDGYMFLKAESKIMFATLQRSSLWCLCSNH (Minimal epitopes KPKRDGYMF and KPKRDGYMFL analyzed). FIG. 10B depicts an exemplary result of a T cell response assay using PBMCs from an HLA-A02:01 healthy donor showing fraction of antigen specific CD8⁺ T cells that responded to stimulating peptide SMLTGPPARVPAVPFDLH (Minimal epitopes SMLTGPPARV and MLTGPPARV analyzed).

### Example 11—Cytotoxicity Assay of Induced T Cells

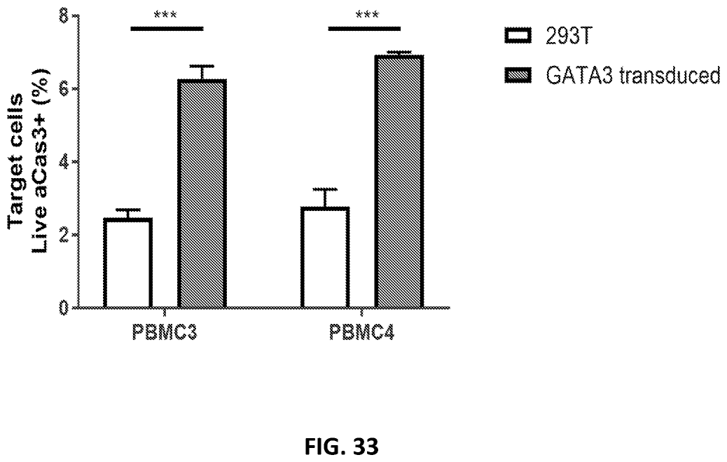

A cytotoxicity assay was used to assess whether the induced T cell cultures can kill antigen expressing tumor lines. In this example, expression of active caspase 3 on alive and dead tumor cells was measured to quantify early cell death and dead tumor cells. In FIG. 33, the induced CD8⁺ responses were capable of killing antigen expressing tumor targets. The percent live caspase-A positive target cells of two samples mock transduced or transduced with a lentiviral expression vector encoding a GATA3 neoORF peptide are shown.

### Example 12—Peptide Synthesis

The peptides in Table 5 below were synthesized and purified. The predicted and determined molecular weights are shown. Also shown are the crude purities and final purities for the indicated peptides.

TABLE 5

| | | Theoretical | Determined | Crude | Final |
|---|---|---|---|---|---|
| ID | Sequences | MW | MW | Purity | Purity |
| L7 | EPCSMLTGPPARVPAVPFDLH | 2234.6 | 2235.2 | 47% | 97.4% |
| L8 | GPPARVPAVPFDLHFCRSSIMKPKRD | 2922.5 | 2923.5 | 51% | 99.5% |
| L9 | LHFCRSSIMKPKRDGYMFLKAESKI | 2986.6 | 2987.7 | 37% | 98.1% |
| L10 | KPKRDGYMFLKAESKIMFATLQR | 2759.3 | 2760.3 | 53% | 92.6% |
| L10b | KPKRDGYMFLKAESKIMFAT | 2361.9 | 2362.4 | 41% | 97.6% |
| L10b-4K | KKKKKPKRDGYMFLKAESKIMFAT | 2874.5 | 2874.9 | 57% | 85.7% |
| L10c | KPKRDGYMFLKAESKI | 1911.3 | 1911.4 | 69% | 98.4% |
| L11 | FLKAESKIMFATLQRSSLWCL | 2473.0 | 2473.0 | 66% | 86% |
| L11b | YMFLKAESKIMFATLQRSSLWCL | 2767.4 | 2767.2 | 37% | 80.0% |
| L11c | YMFLKAESKIMFATLQRSS | 2251.7 | 2252.4 | 38% | 95.9% |
| L11c-4K | KKKKYMFLKAESKIMFATLQRSS | 2764.3 | 2764.8 | 45% | 82.0% |
| L11d | KAESKIMFATLQRSSLWCL | 2212.7 | 2213.0 | 32% | 96.6% |
| L11d-4K | KKKKKAESKIMFATLQRSSLWCL | 2725.3 | 2725.8 | 28% | 82.3% |
| L11e | DGYMFLKAESKIMFAT | 1852.2 | 1852.3 | 39% | 91.6% |
| L11f | FLKAESKIMFATLQRS | 1870.2 | 1870.2 | 62% | 95.0% |
| L11g | ESKIMFATLQRSSLWC | 1900.2 | 1900.2 | 41% | 91.0% |
| L11h | FLKAESKIMFATLQR | 1783.1 | 1783.8 | 35% | 84% |
| L11i | ESKIMFATLQRSSL | 1610.87 | 1610.80 | 75% | 97% |
| L12 | KIMFATLQRSSLWCLCSNH | 2238.7 | 2238.6 | 31% | 68.0% |
| L12-4K | KKKKKIMFATLQRSSLWCLCSNH | 2751.3 | 2751.8 | 49% | 75.7% |
| L12b | MFATLQRSSLWCLCSNH | 1997.3 | 1997.8 | 47% | 98.7% |
| L12b-4K | KKKKMFATLQRSSLWCLCSNH | 2510 | 2510.4 | 39% | 92.7% |
| L12c | MFATLQRSSLWCLC | 1659.0 | 1659 | 57% | 90% |
| L12d | TLQRSSLWCLCSNH | 1647.9 | 1648 | 60% | 99% |
| L14 | SMLTGPPARVPAVPFDLH | 1905.2 | 1905.3 | 64.5% | 99.5% |
| L15 | KPKRDGYMFLKAESKIMFATLQRSSL | 3890.58 | 3891 | 37% | 96% |
| | WCLCSNH | | | | |
| L15b | KPKRDGYMFLKAESKIMFATLQRSSL | 3449.1 | 3449.5 | 42% | 87% |
| | WCL | | | | |
| L15c | DLHFCRSSIMKPKRDGYMFLKAESKIM | 4639.5 | 4640.4 | 40% | 90% |
| | FATLQRSSLWCL | | | | |

### Example 13—Solubility Tests of Synthetic GATA3 neoORF Peptide

The solubility of each peptide in Table 6 below was tested in the various indicated solutions. SS=sodium succinate. Formulation A tested included 4 DMSO, 5 mM sodium succinate (SS) in D5W. Formulation B tested included no DMSO, 5 mM SS in D5W. Formulation C tested included no DMSO, 0.25 mM SS in D5W. Synthesis of the 33mer L15, which contains two cysteies, was carried out using by creating pseudo-proline building blocks through conjugation of the side chains of ES, AT and SS in the sequence. This allowed for L15 to be purified to 95% purity and prevented aggregation during solid phase peptide synthesis.

### Table 6 Below Lists Peptide Solubilities

| | | | | | | | Poly | Poly | Poly | Poly | | Poly | | Poly |
|---|---|---|---|---|---|---|---|---|---|---|---|---|---|---|
| | | | | | | | ICLC | ICLC | ICLC | ICLC | | ICLC | | ICLC |
| | | | | | | | + | + | + | + | | + | 5 | + |
| | | | 5 | 0.5 | 0.75 | 0.25 | 0.5 | 0.75 | 0.25 | 5 | 0.25 | 0.25 | mM | 5 |
| | | | mM | mM | mM | mM | mM | mM | mM | mM | mM | mM | SS | mM |
| | | | SS in | SS in | SS in | SS in | SS in | SS in | SS in | SS in | SS in | SS in | in | SS in |
| | | | D5W | D5W | D5W | D5W | D5W | D5W | D5W | D5W | D5W | D5W | D5W | D5W |
| | | | w/ 4% | w/ 4% | w/ 4% | w/ 4% | w/ 4% | w/ 4% | w/ 4% | w/ 4% | w/o | w/o | w/o | w/o |
| ID | Sequence | AA | DMSO | DMSO | DMSO | DMSO | DMSO | DMSO | DMSO | DMSO | DMSO | DMSO | DMSO | DMSO |
| 7 | EPCSMLTGP | 21 | Yes | Yes | Yes | Yes | Yes | Yes | Yes | | Yes | Yes | Yes | Yes |
| | PARVPAVPF | | | | | | | | | | | | | |
| | DLH | | | | | | | | | | | | | |
| 14 | SMLTGPPAR | 18 | Yes | Yes | Yes | Yes | Yes | Yes | Yes | Yes | | | Yes | Yes |
| | VPAVPFDLH | | | | | | | | | | | | | |
| 8 | GPPARVPAV | 26 | Yes | Yes | Yes | Yes | Yes | Yes | Yes | | Yes | Yes | Yes | Yes |
| | PFDLHFCRS | | | | | | | | | | | | | |
| | SIMKPKRD | | | | | | | | | | | | | |
| 9 | LHFCRSSIM | 25 | Yes | Yes | Yes | Yes | | Yes | Yes | | Yes | | Yes | Yes |
| | KPKRDGYMF | | | | | | | | | | | | | |
| | LKAESKI | | | | | | | | | | | | | |
| 10 | KPKRDGYMF | 23 | Yes | | | Yes | | No | | No | | | | |
| | LKAESKIMF | | | | | | | | | | | | | |
| | ATLQR | | | | | | | | | | | | | |
| 11 | FLKAESKIM | 21 | | | | | | | No | | | | | |
| | FATLQRSSL | | | | | | | | | | | | | |
| | WCL | | | | | | | | | | | | | |
| 12 | KIMFATLQR | 19 | | | | | | | | | | | | |
| | SSLWCLCSN | | | | | | | | | | | | | |
| | H | | | | | | | | | | | | | |
| 10b | KPKRDGYMF | 20 | | Yes | Yes | Yes | | No | No | | | | | |
| | LKAESKIMF | | | | | | | | | | | | | |
| | AT | | | | | | | | | | | | | |
| 11b | YMFLKAESK | 23 | | | | | | | | | | | | |
| | IMFATLQRS | | | | | | | | | | | | | |
| | SLWCL | | | | | | | | | | | | | |
| 11c | YMFLKAESK | 19 | | Yes | Yes | Yes | | No | No | | | | | |
| | IMFATLQRS | | | | | | | | | | | | | |
| | S | | | | | | | | | | | | | |
| 11d | KAESKIMFA | 19 | | Yes | Yes | Yes | | | | | Yes | Yes | | |
| | TLQRSSLWC | | | | | | | | | | | | | |
| | L | | | | | | | | | | | | | |
| 12b | MFATLQRSS | 17 | | Yes | Yes | Yes | | No | No | | | | | |
| | LWCLCSNH | | | | | | | | | | | | | |
| 10b- | KKKKKPKRD | 25 | Yes | | | | | | | | | | | |
| 4K | GYMFLKAES | | | | | | | | | | | | | |
| | KIMFAT | | | | | | | | | | | | | |
| 11c- | KKKKYMFLK | 23 | | | | | | | | | | | | |
| 4K | AESKIMFAT | | | | | | | | | | | | | |
| | LQRSS | | | | | | | | | | | | | |
| 11d- | KKKKKAESK | 23 | Yes | | | | | | | | | | | |
| 4K | IMFATLQRS | | | | | | | | | | | | | |
| | SLWCL | | | | | | | | | | | | | |
| 12b- | KKKKMFATL | 20 | Yes | | | | | | | | | | | |
| 4K | QRSSLWCLC | | | | | | | | | | | | | |
| | SNH | | | | | | | | | | | | | |
| 12- | KKKKKIMFA | 23 | Yes | | | | | | | | | | | |
| 4K | TLQRSSLWC | | | | | | | | | | | | | |
| | LCSNH | | | | | | | | | | | | | |
| L10c | KPKRDGYMF | 16 | Yes | | | Yes | | | | Yes | Yes | Yes | Yes | Yes |
| | LKAESKI | | | | | | | | | | | | | |
| L11e | DGYMFLKAE | 16 | No | | | No | | | | | | | | |
| | SKIMFAT | | | | | | | | | | | | | |
| L11f | FLKAESKIM | 16 | | | Yes | Yes | | | | | Yes | Yes | | |
| | FATLQRS | | | | | | | | | | | | | |
| L11g | ESKIMFATL | 16 | No | | | Yes | | | No | | | | | |
| | QRSSLWC | | | | | | | | | | | | | |
| L11h | FLKAESKIM | 15 | | | | | | | | | | | | |
| | FATLQR | | | | | | | | | | | | | |
| L11i | ESKIMFATL | 14 | | | | | | | | | Yes | Yes | | |
| | QRSSL | | | | | | | | | | | | | |
| L12c | MFATLQRSS | 14 | No | | | No | | | | | | | | |
| | LWCLC | | | | | | | | | | | | | |
| L12d | TLQRSSLWC | 14 | Yes | | | Yes | | | Yes | Yes | Yes | | | |
| | LCSNH | | | | | | | | | | | | | |
| L15 | KPKRDGYMF | 33 | No | | | | | | | | Yes | Yes | | |
| | LKAESKIMF | | | | | | | | | | | | | |
| | ATLQRSSLW | | | | | | | | | | | | | |
| | CLCSNH | | | | | | | | | | | | | |
| L15b | KPKRDGYMF | 29 | | | | | | | | | | | | |
| | LKAESKIMF | | | | | | | | | | | | | |
| | ATLQRSSLW | | | | | | | | | | | | | |
| | CL | | | | | | | | | | | | | |
| L15c | DLHFCRSSI | 39 | | | | | | | | | | | | |
| | MKPKRDGYM | | | | | | | | | | | | | |
| | FLKAESKIM | | | | | | | | | | | | | |
| | FATLQRSSL | | | | | | | | | | | | | |
| | WCL | | | | | | | | | | | | | |
| 10b- | KKKKKPKRD | 24 | Yes | | | | | | | | | | | |
| 4K | GYMFLKAES | | | | | | | | | | | | | |
| | KIMFAT | | | | | | | | | | | | | |
| 11c- | KKKKYMFLK | 23 | | | | | | | | | | | | |
| 4K | AESKIMFAT | | | | | | | | | | | | | |
| | LQRSS | | | | | | | | | | | | | |
| 11d- | KKKKKAESK | 23 | Yes | | | | | | | | | | | |
| 4K | IMFATLQRS | | | | | | | | | | | | | |
| | SLWCL | | | | | | | | | | | | | |
| 12b- | KKKKMFATL | 21 | Yes | | | | | | | | | | | |
| 4K | QRSSLWCLC | | | | | | | | | | | | | |
| | SNH | | | | | | | | | | | | | |
| 12- | KKKKKIMFA | 23 | Yes | | | | | | | | | | | |
| 4K | TLQRSSLWC | | | | | | | | | | | | | |
| | LCSNH | | | | | | | | | | | | | |

### Example 14—Design of Pools of Synthetic GATA3 neoORF Peptide for Administration to Subjects

Various pools of the indicated GATA3 peptides were designed according to Table 7 below. For example, “Design 1” contains three peptide pools where pool 1 contains three peptides (i.e., L7, L8 and L14), pool 2 contains two peptides (i.e., L9 and L10c) and pool 3 contains two peptides (i.e., L15 and L11f). For example, “Design 6” contains two peptide pools where pool 1 contains four peptides (i.e., L7, L8, L9 and L14) and pool 2 contains two peptides (i.e., L15 and L11f). For example, “Design 10” contains four peptide pools where pool 1 contains five peptides (i.e., L7, L8, L9, L10c and L14), pool 2 contains one peptide (i.e., L15), pool 3 contains one peptide (i.e., L11f) and pool 4 contains one peptide (i.e., L11i). The concentration of each peptide in the pools can be changed according to one skilled in the art of preparing peptide formulations. Table 7 below lists description of GATA3 pool designs.

TABLE 7

| | | Design 1 | Design 2 | Design 3 | Design 4 | Design 5 | Design 6 | Design 7 | Design 8 | Design 9 | Design 10 | Design 11 |
|---|---|---|---|---|---|---|---|---|---|---|---|---|
| # | | 3 pools | 3 pools | 3 pools | 2 pools | 4 pools | 2 pools | 3 pools | 4 pools | 3 pools | 4 pools | 3 pools |
| 1 | L7 | 1 | 1 | 1 | 1 | 1 | 1 | 1 | 1 | 1 | 1 | 1 |
| 2 | L14 | 1 | 1 | 1 | 1 | 1 | 1 | 1 | 1 | 1 | 1 | 1 |
| 3 | L8 | 1 | 2 | 2 | 1 | 2 | 1 | 2 | 1 | 1 | 1 | 1 |
| 4 | L9 | 2 | 2 | 2 | 1 | 2 | 1 | 2 | 1 | 1 | 1 | 1 |
| 5 | L15 | 3 | 3 | 3 | 2 | 4 | 2 | 3 | 2 | 2 | 2 | 2 |
| 6 | L11f | 3 | 3 | 3 | 2 | 3 | 2 | 3 | 3 | 3 | 3 | N/A |
| 7 | L10c | 2 | 2 | 1 | 1 | 2 | N/A | N/A | 4 | 1 | 1 | 1 |
| 8 | L11i | N/A | N/A | N/A | N/A | N/A | N/A | N/A | N/A | N/A | 4 | 3 |

### Example 15—GATA3 neoORF Peptide Syntheses

Conventional synthesis is performed with a target of 700 mg crude material. The following Fmoc-amino acids with proper side chain protections were used in constructing L7 peptide (EPCSMLTGPPARVPAVPFDLH): Fmoc-Ala-OH.H₂O, Fmoc-Cys(Trt)-OH, Fmoc-Asp(OtBu)-OH, Fmoc-Glu(OtBu)-OH, Fmoc-Gly-OH, Fmoc-Leu-OH, Fmoc-Met-OH, Fmoc-Pro-OH, Fmoc-Arg(Pbf)-OH, Fmoc-Ser(tBu)-OH, Fmoc-Thr(tBu)-OH, and Fmoc-Val-OH. C-terminal histidine was incorporated into the sequence by being preloaded onto the resin by using either H-His(Trt)-2Cl-Trt resin or Fmoc-His(Trt)-Wang resin. Fmoc-Asp(OMpe)-OH may be used in place of Fmoc-Asp(OtBu)-OH to help improve synthesis, such as with sequence combinations of “DG” to minimize aspartamide formation.

The peptide sequences were swelled with dimethylformamide (DMF) and drained twice. Synthesis began with the deprotection of the N-α-FMOC protecting group using 20% piperidine in DMF with nitrogen dispensing to mix. After draining, the resin was washed with DMF. Next, a 0.4 M amino acid solution was added along with 0.4 M HCTU and 0.8 M DIEA. The coupling reaction was run with nitrogen dispensing to mix, followed by draining the reaction vessel (RV). The amino acid, HCTU, and DIEA additions were repeated for a double coupling cycle with the same mixing and draining parameters as the first coupling step. The resin was then washed with DMF again. This cycle was repeated for every amino acid residue. The final deprotection method removed the N-terminal Fmoc via 20% piperidine in DMF, and the resin was washed with DMF followed by washes with MeOH. The resin was on the instrument under nitrogen until removed.

For microwave synthesis, the same Fmoc-amino acid starting materials were used, with only the Fmoc-His(Trt)-Wang resin (but not H-His(Trt)-2Cl Trt resin) utilized to incorporate the C-terminal histidine.

On the microwave synthesizer, resin was swelled in DMF until it was transferred through the HT lines to the microwave reaction vessel (RV). While in the RV, the Fmoc-His(Trt)-OH loaded resin was treated with 25% pyrrolidine in DMF to remove the N-α-FMOC under 85° C./90 W followed by 100° C./20 W. Next, the RV was drained and washed with DMF, and drained again. The programmed Fmoc-amino acid was added (0.5 M in DMF) to the RV along with 4M DIC and 0.25 M Oxymapure. This coupling reaction followed 105° C./288 W heating followed by 105° C./73 W heating. This first deprotection was initially diluted with DMF, however this step was not required for any subsequent deprotections as the RV already contained DMF from the coupling reaction. The deprotection, wash, and coupling cycles were repeated for each residue until the peptide had been synthesized. For arginine residues, a double coupling step was performed, where after the single coupling was performed, the solution was drained, and the coupling step was repeated before proceeding to the deprotection. The final deprotection of the N-terminal Fmoc group was performed as above, except for being drained and washed twice with DMF before being transferred via DMF back to the original HT resin position.

After synthesis, the resin was transferred to a fritted syringe using DMF, rinsed with MeOH, and dried using a vacuum manifold. Then the resin was cleaved using Reagent K (82.5% trifluoroacetic acid (TFA), 5% water, 5% thioanisole, 5% phenol, and 2.5% ethanedithiol) using an upright holder on an oscillating shaker for three hours at room temperature.

The cleavage cocktail was then dispensed through a filtered syringe frit into cold diethyl ether or cold methyl tert-butyl ether (MTBE). Each syringe was then rinsed with a 95:5 trifluoroacetic acid:water solution by agitation. The rinse was then added to the rest of the cocktail/ether mixture. The mixture was then centrifuged. After decanting the ether, another cold ether wash was added. The container was vortexed and centrifuged again. This was repeated to thoroughly rinse the pellet. The final wash was decanted and the pellet dried via vacuum desiccator. A sample of the pellet was dissolved in solvent (e.g., DMSO, DMF, water, or acetonitrile) and analyzed via UPLC-MS for identity, crude purity, and retention time. Other peptides, for example L14 (SMLTGPPARVPAVPFDLH), L8 (GPPARVPAVPFDLHFCRSSIMKPKRD), L10c (KPKRDGYMFLKAESKI), L11h (FLKAESKIMFATLQR), and L11i (ESKIMFATLQRSSL) were made in a similar fashion, using amino acids and pre-loaded resins specific to those sequences.

### Example 16—GATA3 neoORF Peptide Syntheses

The following Fmoc-amino acids were used in synthesizing peptide L15 (KPKRDGYMFLKAESKIMFATLQRSSLWCLCSNH): Fmoc-Ala-OH.H₂O, Fmoc-Cys(Trt)-OH, Fmoc-Asp(OtBu)-OH, Fmoc-Glu(OtBu)-OH, Fmoc-Phe-OH, Fmoc-Gly-OH, Fmoc-Ile-OH, Fmoc-Lys(Boc)-OH, Fmoc-Leu-OH, Fmoc-Met-OH, Fmoc-Asn(Trt)-OH, Fmoc-Pro-OH, Fmoc-Gln(Trt)-OH, Fmoc-Arg(Pbf)-OH, Fmoc-Ser(tBu)-OH, Fmoc-Thr(tBu)-OH, Fmoc-Trp(Boc)-OH, and Fmoc-Tyr(tBu)-OH. C-terminal histidine was incorporated into the sequence by being preloaded onto the resin by using either H-His(Trt)-2Cl-Trt resin or Fmoc-His(Trt)-Wang resin. Fmoc-Asp(OMpe)-OH may be used in place of Fmoc-Asp(OtBu)-OH to help improve synthesis, such as with sequence combinations of “DG” to minimize aspartamide formation. Where serine (Ser, S) and threonine (Thr, T) residues are present, amino acid dipeptides (psuedoprolines) were incorporated to improve synthesis yields, such as Fmoc-Ser(tBu)-Ser(psi(Me,Me)pro)-OH in place of “SS”, Fmoc-Ala-Thr(psi(Me,Me)pro)-OH in place of “AT”, and Fmoc-Glu(OtBu)-Ser(psi(Me,Me)pro)-OH in place of “ES”. For some syntheses of L15, the combination of all three pseudoprolines (i.e., Fmoc-Ser(tBu)-Ser(psi(Me,Me)pro)-OH in place of “SS”, Fmoc-Ala-Thr(psi(Me,Me)pro)-OH in place of “AT”, and Fmoc-Glu(OtBu)-Ser(psi(Me,Me)pro)-OH) in place of “ES”) was used. For other syntheses of L15, the following pesudoproline and pseudoproline combinations were used, respectively: Fmoc-Ala-Thr(psi(Me,Me)pro)-OH in place of “AT” and Fmoc-Glu(OtBu)-Ser(psi(Me,Me)pro)-OH in place of “ES”; Fmoc-Ser(tBu)-Ser(psi(Me,Me)pro)-OH in place of “SS” and Fmoc-Glu(OtBu)-Ser(psi(Me,Me)pro)-OH in place of “ES”; Fmoc-Ser(tBu)-Ser(psi(Me,Me)pro)-OH in place of “SS” and Fmoc-Ala-Thr(psi(Me,Me)pro)-OH in place of “AT”; Fmoc-Ala-Thr(psi(Me,Me)pro)-OH in place of “AT”; Fmoc-Glu(OtBu)-Ser(psi(Me,Me)pro)-OH) in place of “ES”; and Fmoc-Ser(tBu)-Ser(psi(Me,Me)pro)-OH in place of “SS”.

Peptide sequences were swelled with DMF and drained twice. Synthesis began with the deprotection of the N-α-Fmoc group using 20% piperidine in DMF with nitrogen dispensing to mix. After draining, the resin was washed with DMF. Next, 0.4 M amino acid solution was added along with 0.4 M HCTU and 0.8 M DIEA. The coupling reaction was carried out with nitrogen dispensing to mix, followed by draining the reaction vessel (RV). The amino acid, HCTU, and DIEA additions were repeated for a double coupling cycle with the same mixing and draining parameters as the first coupling step. The resin was then washed with DMF again. This cycle was repeated for every amino acid residue. The final deprotection method removed the N-terminal Fmoc via 20% piperidine in DMF, and the resin was washed with DMF followed by washes with MeOH. The resin was on the instrument under cover of nitrogen until removed.

For microwave synthesis, the same amino acid starting materials were used, with only the Fmoc-His(Trt)-Wang resin (but not H-His(Trt)-2Cl-Trt resin) utilized to incorporate the C-terminal histidine.

On the microwave synthesizer, resin was swelled in DMF until it was transferred through the HT lines to the microwave reaction vessel (RV). While in the RV, the Fmoc-His(Trt) loaded resin was treated with 25% pyrrolidine in DMF to remove the N-α-Fmoc under 85° C./90 W followed by 100° C./20 W. This first deprotection was initially diluted with DMF; however, this step was not required for any subsequent deprotections as the RV already contained DMF from the coupling reaction. Next the RV was drained and washed with DMF, and drained again. The programmed Fmoc-amino acid was then added (0.5 M in DMF) to the RV along with 4 M DIC and 0.25 M Oxymapure. This coupling reaction followed 105° C./288 W heating followed by 105° C./73 W heating. The deprotection, wash, and coupling cycles were repeated for each residue until the peptide had been synthesized. For arginine residues, there was a double coupling step, where after the single coupling was performed, the solution was drained, and the coupling step was repeated before proceeding to the deprotection. The final deprotection of the N-terminal Fmoc group was performed the same as all other deprotections steps, except for being drained and washed twice with DMF before being transferred via DMF back to the original HT resin position.

After synthesis, the resin was transferred to a fritted syringe using DMF, rinsed with MeOH, and dried using vacuum manifold. The resin was then cleaved using Reagent K (82.5% trifluoroacetic acid (TFA), 5% water, 5% thioanisole, 5% phenol, and 2.5% ethanedithiol) using an upright holder on an oscillating shaker at room temperature.

The cleavage cocktail was then dispensed through filtered syringe frit into cold diethyl ether (or cold MTBE). Each syringe was then rinsed with a 95:5 trifluoroacetic acid: water solution by agitation. The rinse was then added to the rest of the cocktail/ether mixture. Then the mixture was centrifuged. After decanting the ether, another cold ether wash was added. The container was vortexed and centrifuged again. This was repeated to thoroughly rinse the pellet. The final wash was decanted and the pellet was dried via vacuum desiccator. A sample of the pellet was dissolved in solvent (e.g., DMSO, DMF, water, or acetonitrile) and analyzed via UPLC-MS for identity, crude purity, and retention time.

Other peptides, for example L9 (LHFCRSSIMKPKRDGYMFLKAESKI), were made in a similar fashion, using amino acids and pre-loaded resins specific to those sequences, as well as pseudoproline derivatives where serine (Ser, S) or threonine (Thr, T) residues are present and Fmoc-Asp(OMpe)-OH as described above.

### Example 17—Solubility Studies

A number of GATA3 peptides with neoepitopes were first tested for solubility using 5 mM sodium succinate (SS) in D5W with 4% DMSO. Based on the initial results the formulation strategy was improved by adjusting the sodium succinate (SS) concentration and DMSO amount, which lead to the selection of 7 peptides. The pooling strategies of these peptides were determined for solubility and compatibility with polyICLC. Based on these results, three pools were selected. Two pools each with only one peptide in 0.25 mM SS in D5W and a third pool with 5 peptides in 5 mM SS in D5W. The pH of the pools after being combined with polyICLC were all pH 5.0-6.0 and there was minimal loss during filtration.

The peptides screened for the following studies are all listed in Table 8.

TABLE 8

| | | | | Theo- | |
|---|---|---|---|---|---|
| | | | Theo- | retical | |
| | | Molec- | retical | % | |
| | | ular | TFA | peptide | % |
| Name | Sequence | Weight | content | content | purity |
| L7 | EPCSMLTGPPA | 2234.6 | 13.3 | 80.3 | 97 |
| | RVPAVPFDLH | | | | |
| L8 | GPPARVPAVPF | 2922.4 | 21.5 | 72.1 | 100 |
| | DLHFCRSSIMK | | | | |
| | PKRD | | | | |
| L9 | LHFCRSSIMKP | 2986.6 | 23.4 | 70.2 | 98 |
| | KRDGYMFLKAE | | | | |
| | SKI | | | | |
| L10B | KPKRDGYMFLK | 2361.8 | 22.5 | 71.1 | 98 |
| | AESKIMFAT | | | | |
| L11C | YMFLKAESKIM | 2251.7 | 16.8 | 76.8 | 93 |
| | FATLQRSS | | | | |
| L11D | KAESKIMFATL | 2212.6 | 17.1 | 76.5 | 92 |
| | QRSSLWCL | | | | |
| L12B | MFATLQRSSLW | 1997.3 | 14.6 | 79.0 | 93 |
| | CLCSNH | | | | |
| L10 | KPKRDGYMFLK | 2759.3 | 22.4 | 71.2 | 93 |
| | AESKIMFATLQ | | | | |
| | R | | | | |
| L10c | KPKRDGYMFLK | 1911.3 | 26.4 | 67.2 | 98 |
| | AESKI | | | | |
| L11 | FLKAESKIMFA | 2473.0 | 15.6 | 78.0 | 86 |
| | TLQRSSLWCL | | | | |
| L11e | DGYMFLKAESK | 1852.2 | 15.6 | 78.0 | 92 |
| | IMFAT | | | | |
| L11f | FLKAESKIMFA | 1870.2 | 19.6 | 74.0 | 95 |
| | TLQRS | | | | |
| L11g | ESKIMFATLQR | 1900.2 | 15.3 | 78.3 | 91 |
| | SSLWC | | | | |
| L12c | MFATLQRSSLW | 1659.0 | 12.1 | 81.5 | 90 |
| | CLC | | | | |
| L12d | TLQRSSLWCLC | 1647.9 | 17.2 | 76.4 | 99 |
| | SNH | | | | |
| L15 | KPKRDGYMFLK | 3890.6 | 19.0 | 74.6 | 98 |
| | AESKIMFATLQ | | | | |
| | RSSLWCLCSNH | | | | |
| L14 | SMLTGPPARVP | 1905.2 | 15.2 | 78.4 | 99 |
| | AVPFDLH | | | | |
| L11i | ESKIMFATLQR | 1610.9 | 17.5 | 76.1 | 96 |
| | SSL | | | | |

All buffers were prepared daily. D5W was prepared by weighing the dextrose and adding milliQ water to the dextrose to reach the appropriate volume. For example, water was added to 12.5 g dextrose to reach a total volume of 250 mL. To prepare 50 mL 5 mM SS in D5W by weighing 67.54 mg SS was weighed and added to D5W to reach 50 mL total volume. To prepare 0.25 mM SS in DSW, 2.5 mL of 5 mM SS in D5W was diluted with 47.5 mL of DSW.

The 00 peptide content of each peptide was determined as follows: The total theoretical TFA is equal to the sum of the number of positive charges (N-terminus, Arg, Lys, and His). That number was entered in to the following equation where MW is the molecular weight of the peptide:

00 TFA=100*((0% TFA*114.02)/((% TFA*114.02)+MW)) Equation 1.

This value was then used to calculate the percent peptide content using 6.45% as theoretical water content:

% Peptide=100−% TFA−6.45% Equation 2.

The target gross weight for these experiments was calculated using the equation below.

Target gross weight=(13.2*10000)/(% peptide content*% purity) Equation 3.

Peptides were weighed into 15 mL or 50 mL conical tubes using a Mettler Toledo XP105 Delta Mass analytical balance and the actual gross weight was recorded and used to determine how much DMSO to obtain 50 mg/mL. The calculation is shown below:

DMSO (μL)=(Actual gross weight (mg)*264 μl)/(Target gross weight (mg)) Equation 4.

The stock was then diluted to 2 mg/mLL (1 part DMSO stock, 24 parts buffer) in the appropriate formulation buffer.

Peptides weights and percent content were calculated as described above and the appropriate buffer was added directly to the peptides. The target gross weight calculated using Eq. 3 and the volume of buffer used to obtain 2 mg/mL peptide was calculated using Equation 5.

Buffer (ml)=(Actual gross weight (mg)*6.6 mL)/(Target gross weight (mg)) Equation 5.

Peptides were further diluted 1:4 with buffer to obtain 0.4 mg/mL or, only when indicated, was buffer added directly to the dry peptide to obtain 0.4 mg/mL. In the latter case, Equation 6 to determine the appropriate volume to add.

Buffer (ml)=(Actual gross weight (mg)*33 mL)/(Target gross weight (mg)) Equation 6.

The peptides were dissolved by inverting the conical tubes and not by sonicating or vortexing them.

To pool 5 peptides, equal volume from each 2 mg/mL stock was combined to obtain 0.4 mg/mL of each peptide. In the case of pools with less than 5 peptides, equal volumes of the peptides were combined then the solution was diluted with the appropriate buffer to obtain 0.4 mg/mL of each peptide. The pools were inverted 3-5 times to mix. The formulated peptides were transferred to glass vials to visualize solubility. Photographs were taken every two hours to note any changes in appearance.

PolyICLC was obtained commercially. Pools were combined at a 3:1 ratio of peptide to polyICLC using 150 μL polyICLC with 450 μL peptide pool in a 2 mL glass vial. The solution was inverted 3-5 times to mix and photographs were taken every two hours for 6 hours to note any changes in appearance. All pH measurements were made using a Mettler Toledo inLab Micro pH meter, which was calibrated every day before use. 100 μL of the sample being analyzed was removed and added to a microcentrifuge tube to measure the pH. The sample was then discarded. Samples by UPLC-MS (Waters Acquity H-Class with an Acquity QDa mass spectrometer). A 2 μL injection of each sample was analyzed in duplicate using an 8 minute gradient from 10:90 solvent A:B to 50:50 solvent A:B (A:0.1% TFA/water, B: 0.1% TFA:acetonitrile). Initial solubility of peptides in the standard formulation was determined for each peptide at 0.4 mg/mL and 2 mg/mL. Though photographs were taken, they did not always clearly show solubility since gels were often clear or the peptides for small glassy particulates when dissolved. The peptide solubilities in 5 mM SS/D5W with 4% DMSO are indicated in Table 9.

Table 9 Below Lists Peptide Solubilities and Observations in 5 mM SS/D5W with 4% DMSO

| | Peptide | | Peptide | |
|---|---|---|---|---|
| | Solubility | Formulation | Solubility | Formulation |
| Peptide | (at 2 mg/mL) | Observations | (at 0.4 mg/mL) | Observations |
| Name | (Y/N) | (at 2 mg/mL) | (Y/N) | (at 0.4 mg/mL) |
| L7 | Y | clear | Y | clear |
| L8 | Y | clear | Y | clear |
| L9 | Y | clear | Y | clear |
| L15 | N | cloudy | N | Cloudy over |
| | | | | 4 hours |
| L14 | Y | clear | Y | clear |
| L10 | Y | clear | Y | clear |
| L10c | Y | clear | Y | clear |
| L11 | N | cloudy, | N | Glassy |
| | | precipitation | | particulates |
| L11e | N | cloudy, large | N | Glassy |
| | | precipitates | | particulates |
| L11f | N | cloudy | N | Glassy |
| | | | | particulates |
| L11g | N | cloudy | N | Glassy |
| | | | | particulates |
| L12c | N | cloudy, large | N | Glassy |
| | | precipitates | | particulates |
| L12d | Y | clear | Y | clear |

Peptides that were insoluble in 5 mM SS were tested using 0.25 mM SS in D5W with 4% DMSO. The results are summarized in Table 10. Many of these peptides that were insoluble in 5 mM SS, were soluble at a concentration of 0.4 mg/mL in 0.25 mM SS/D5W with 4% DMSO after 6 hours and were tested in further studies using this lower SS concentration.

Table 10 lists Peptide Solubilities and Observations in 0.25 mM SS/D5W with 4% DMSO

| | Peptide | | Peptide | |
|---|---|---|---|---|
| | Solubility | Formulation | Solubility | Formulation |
| Peptide | (at 2 mg/mL) | Observations | (at 0.4 mg/mL) | Observations |
| Name | (Y/N) | (at 2 mg/mL) | (Y/N) | (at 0.4 mg/mL) |
| L15 | Y | clear | Y | clear |
| L11 | N (glassy | glass | Y | clear |
| | particulates) | particulates | | |
| L11e | N | gel | N | glassy |
| | | | | particulates |
| L11f | Y | clear | Y | clear |
| L11g | N (gel over | gel over time | Y | clear |
| | time) | | | |
| L12c | N | large | N | cloudy |
| | | particulates | | |
| L12d | Y | clear | Y | clear |

Based on these results peptides L7, L8, L9, L14, L10c, L11d, L11f and L15 were selected for formulation studies. Formulations without DMSO were tested as way to improve stability of formulated peptides and to slow down dimerization of cysteine-containing peptides. All peptides tested (L7, L8, L9, L14, L10c, L11d, L11f and L15) at a concentration of 0.4 mg/mL in 0.25 mM SS and were soluble without DMSO after 6 hours. Peptides L7, L8, L9, L10c, L12d and L14 were tested in 5 mM SS and were also soluble without DMSO after 6 hours. The pH values of the peptide formulations in 0.25 mM SS/D5W and 5 mM SS/D5W are listed in Table 11.

Table 11 Below Shows pH of 0.4 mg/mL Peptide Formulations in 0.25 mM SS/D5W and 5 mM SS/D5W

| Peptide Name | pH in 5 mM SS/D5W | pH in 0.25 mM SS/D5W |
|---|---|---|
| L7 | 6.4 | 4.5 |
| L8 | 6.4 | 5.0 |
| L9 | 6.4 | 4.6 |
| L10c | 6.4 | 4.3 |
| L11f | N/A | 5.0 |
| L14 | 6.4 | 4.2 |
| L15 | N/A | 4.8 |

Because the peptides reported in Table 11 were all soluble in 0.25 mM SS the initial pool designs were studied using the lower SS concentration. The pools were also designed with individual solubility in mind. Because L15 and L11f were soluble in low SS, those two peptides were pooled together. The first three pool designs are shown in Table 12.

### Table 12 Below Shows Initial Peptide Pools in 0.25 mM SS/D5W

| | Design 1 | Design 2 | Design 3 |
|---|---|---|---|
| Peptide name | 3 pools | 3 pools | 3 pools |
| L7 | 1 | 1 | 1 |
| L14 | 1 | 1 | 1 |
| L8 | 1 | 2 | 2 |
| L9 | 2 | 2 | 2 |
| L15 | 3 | 3 | 3 |
| L11f | 3 | 3 | 3 |
| L10c | 2 | 2 | 1 |

All peptides remained soluble after pooling. The pH values of the peptide pools with or without addition of polyICLC are given in Table 13.

Table 13 Below Lists pH of Peptide Pools from Table 12 with or without Addition of PolyICLC

| | | |
|---|---|---|
| Pool | pH of Pool | pH of Pool with polyICLC |

Design 1

| | | |
|---|---|---|
| Pool 1 | 3.4 | 4.7 |
| Pool 2 | 3.8 | 5.1 |
| Pool 3 | 4.0 | 5.5 |

Design 2

| | | |
|---|---|---|
| Pool 1 | 3.7 | 5.0 |
| Pool 2 | 3.6 | 4.8 |
| Pool 3 | 4.0 | 5.5 |

Design 3

| | | |
|---|---|---|
| Pool 1 | 3.5 | 4.7 |
| Pool 2 | 3.9 | 5.5 |
| Pool 3 | 4.0 | 5.5 |

The pools from each design were then mixed with polyICLC to test their compatibility with the adjuvant. Pool 3 and every pool containing peptide L10c precipitated when combined with polyICLC. Additionally, pools with 3 peptides had a pH below 5.0 when combined with polyICLC, which suggests that the buffering capacity should be higher when a pool contains more than two peptides.

Because L7, L8, L9, L10c, and L14 are all soluble in 5 mM SS they were tested in pools with the high SS concentration. Three pools were tested with these five peptides. One pool was without L10c, one had L10c alone, and the third had all five peptides. Because L11f and L15 were not soluble in this higher concentration they were formulated in 0.25 mM SS. However, due to the observed precipitation they were formulated to 0.4 mg/mL separately rather than in a single pool. Each of the peptides was soluble in their respective formulations. These pools were also compatible with polyICLC based on visualization and the pH values after the pools were combined with polyICLC were all between 5.0 and 6.3 (Table 14), which is appropriate for subcutaneous injection.

Table 14 lists pH of Peptide Pools without PolylCLC versus pH of Peptide Pools with PolylCLC

| Pool | pH of pool without polyICLC | pH of pool with polyICLC |
|---|---|---|
| Pool 1 | 5.6 | 5.6 |
| Pool 2 | 5.4 | 5.5 |
| L10c | 6.4 | 6.3 |
| L11f | 5.0 | 5.9 |
| L15 | 4.8 | 6.0 |

Peptide L11i was tested in D5W with various succinate concentrations without DMSO. The peptide appeared soluble in all concentrations of SS at 0.4 mg/mL. Some precipitation was observed at 2 mg/mL in the higher SS concentration. All samples looked the same when combined with polyICLC. The pH values of the formulations of 2 mg/mL or 0.4 mg/mL peptide L11i in 0.25 mM SS, 0.5 mM SS or 5 mM SS without polyICLC and 0.4 mg/mL peptide L11i in 0.25 mM SS 0.5 mM SS or 5 mM SS with polyICLC is shown in Table 15.

Table 15 lists pH of 2 mg/mL or 0.4 mg/mL L11i Peptide in 0.25 mM SS, 0.5 mM SS and 5 mM SS without PolylCLC and 0.4 mg/mL in 0.25 mM SS. 0.5 mM SS and 5 mM SS with PolylCLC

| | pH | | |
|---|---|---|---|
| | 2 mg/mL | 0.4 mg/mL | 0.4 mg/mL peptide L11i |
| | peptide L11i | peptide L11i | with polyICLC |
| 5 mM SS | 5.8 | 6.7 | 6.6 |
| 0.5 mM SS | 4.1 | 5.8 | 6.1 |
| 0.25 mM SS | 3.7 | 5.3 | 6.1 |

The finalized pools based on the results included Pool 1 (L7, L8, L9, L10c, and L14 in 5 mM SS/D5W), pool 2 (either L11i or L11f in 0.25 mM SS/D5W), and pool 3 (L15 in 0.25 mM SS/D5W). Each of these pools was tested for retention on a 0.2 μm filter from Pall (HP1002). The pre-filtered sample as well as sample after each of 2 filtrations were analyzed by UPLC-MS. Less than 3% of L11f and L11i was lost after the first filtration step and no additional peptide was lost after the second filtration step. Only 4.9% L15 was lost after the first filtration and then 1.3% was lost after the second filtration. Less than a total of 3% of each peptide in Pool 1 was lost after the two filtrations steps.

### CONCLUSIONS

A series of potential GATA3 peptides were tested for solubility in the formulation buffer contain 5 mM SS/D5W with 4% DMSO. Peptides that were insoluble in 5 mM SS were also tested in lower SS concentrations. Based on these results seven peptides were selected with five of them being soluble in 5 mM SS (L7, L8, L9, L10c, and L14) and the others being soluble in the lower concentration (L11f, L11i, and L15). Removal of DMSO was also tested, and may improve solubility and slow disulfide formation that can make UPLC analysis more difficult. Each of the peptides selected was soluble without DMSO.

Based on the solubility results, 3 pool designs were generated using 0.25 mM SS/D5W. Although the pools were soluble, some were not compatible with polyICLC. In some instances, precipitation was observed when pools containing L10c were mixed with polyICLC. The same observation was made when the pool containing both L11f and L15 were combined with polyICLC. Based on these results, a fourth set of pools was designed. The first pool contained L7, L8, L9, L10c, and L14 in 5 mM SS/D5W and was compatible with polyICLC. Peptides L15 and L11f or L11i were kept as individual peptides to be prepared by dissolving them directly at 0.4 mg/mL with 0.25 mM SS/D5W. These pools were all above pH 5.0 when mixed with polyICLC, which is acceptable for subcutaneous injection.

### Example 18 Prevalence of GATA3 neoORF Mutation

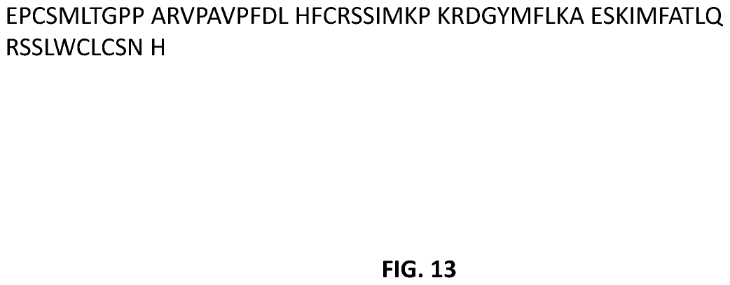

This example characterizes the prevalence and translational evidence of GATA3 neoORF mutation. The vaccine is comprised of a pool of long peptides that span a novel open reading frame in GATA3 (GATA Binding Protein 3) that is present only in cells harboring certain frame-shift mutations in this gene. Depending on the starting position of the frame-shift mutation, the resulting open reading frames may vary in length, but they all share a common translated region “GATA3 neoORF” FIG. 13 provides an exemplary amino acid sequence of a common translated region. Any genetic frame-shift mutations that result in GATA3 neoORF translated sequence is “GATA3 neoORF mutation”. Publically available genomic and proteomic datasets were investigated for prevalence of GATA3 neoORF mutation and evidence of translation for the GATA3 neoORF

### Materials and Methods

### Datasets

MSK-IMPACT breast cancer dataset: The MSK-IMPACT breast cancer dataset (Razavi et al., 2018) is a public dataset available at the cBioPortal for Cancer Genomics (http://www.cbioportal.org/study?id=breast_msk_2018). This dataset contains sequencing data using MSK-IMPACT, a hybridization capture-based next-generation sequencing assay, which analyzes all protein-coding exons between 341 and 468 of cancer-associated genes, from a total of 1918 breast tumor specimens and patient-matched normal from 1756 patients. Publicly available mutation data and clinical data that includes ER status, HER2 status, and overall survival, were downloaded for this study.

TCGA breast cancer proteome dataset: The TCGA breast cancer proteome dataset (NCI CPTAC et al., 2016) is a public dataset available at the CPTAC data portal (https://cptac-data-portal.georgetown.edu/cptac/s/S015). This dataset contains tandem mass spectrometry data from the global proteome of 105 TCGA breast cancer patients using iTRAQ protein quantification methods. Publicly available raw data were downloaded for this study.

### Mutation Prevalence Analysis

GATA3 neoORF identification: Each mutation event of the GATA3 gene from the MSK-IMPACT breast cancer dataset is mapped to the GATA3 transcript ENST00000346208.3 from the human genome (hg19, GRCh37 Genome Reference Consortium Human Reference 37), and then translated in silico into a full-length protein. If a full-length protein contains the GATA3 neoORF sequence, the sample that contains this mutation event is labeled as GATA3 neoORF positive.

GATA3 neoORF prevalence: For all subjects in a cohort, if a subject has at least one sequenced tumor sample that was identified as GATA3 neoORF positive, the subject is considered GATA3 neoORF positive. The GATA3 neoORF prevalence is defined as the percentage of GATA3 neoORF positive subjects in the cohort.

Peptide Identification from Proteomics Data

Protein sequence database: The protein sequence database contains 63,691 protein sequences from the UCSC protein sequence database (the February 2009 human reference sequence, GRCh37/hg19), and one full-length protein that contains the GATA3 neoORF sequence.

Peptide identification: The raw data of the TCGA breast cancer proteome dataset were analyzed with Comet search engine (http://comet-ms.sourceforge.net), an open source software package for interpretation of tandem mass spectra. Comet (version 2017.01 rev.2) was used to search all MS/MS spectra from the TCGA breast cancer proteome dataset against the UCSC protein sequence database. MS/MS spectra with precursor ions up to +6 were allowed in the search. Mass error tolerance for precursor ions was ±10 parts per million (ppm), and a m/z bin width of 0.02 was used for fragment ions. All searches were bounded by trypsin such that each peptide matched to the experimental spectrum had to conform to the cleavage specificity of the enzyme, i.e. C-terminal side of lysines or arginines. A maximum of 2 missed cleavages was allowed. A fixed modification of +144.1021 Da was applied to the N-terminus of a peptide and every Lysine residue as expected for iTRAQ labeling. Variable modifications included up to two oxidized Methionine residues per peptide. A fixed modification of +57.021464 Da was applied to all cysteines for carbamidomethylated cysteines. During the search, decoy peptides were automatically generated as part of the Comet search engine for estimating target-decoy false discovery rates. The search results were processed by Percolator (version 3.02.0) to calculate peptide level q-values, a conventional metric to estimate the false discovery rate of peptide identification using tandem mass spectrometry data. A standard threshold (q-value <0.01) was used to accept peptides identified from the dataset so that less than 1% of the accepted peptides were likely false discoveries.

GATA3 neoORF evidence of translation: Peptides specifically derived from a protein sequence containing the GATA3 neoORF and not from any other protein in the UCSC protein sequence database were called the GATA3 neoORF specific peptides. The identification of GATA3 neoORF specific peptides was considered evidence of translation of the GATA3 neoORF.

### Results

GATA3 neoORF Mutation Prevalence in Breast Cancer

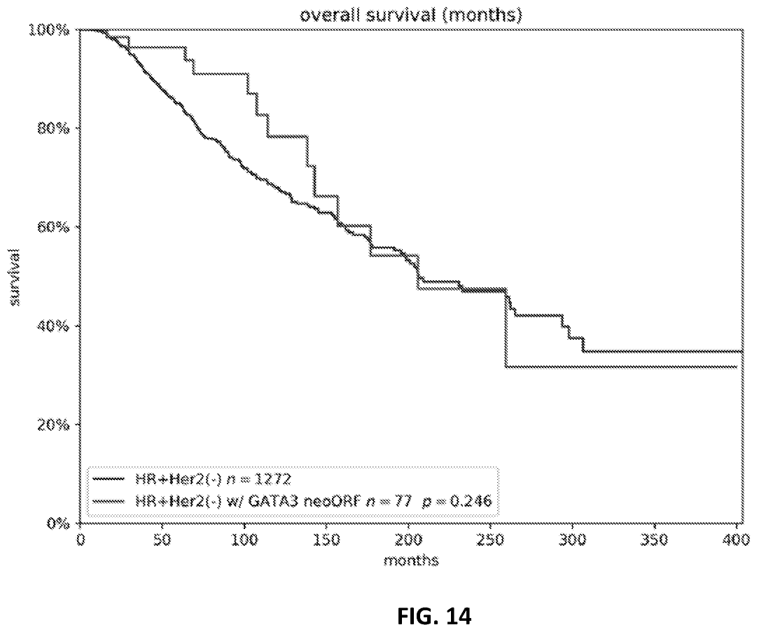

From the MSK-IMPACT breast cancer dataset of 1,756 patients, mutation prevalence analysis was performed (Materials and Methods of Section 1 above) and identified 91 patients that were GATA3 neoORF positive. Of these 91 patients, 77 patients were reported to be HR+Her2(−), and 62 patients were reported to be metastatic at diagnosis. The prevalence of GATA3 neoORF positive patients in each subgroup are reported in Table 16 below. Among the HR+Her2(−) patients, GATA3 neoORF positive patients do not have a statistical difference in overall survival compared to all HR+Her2(−) patients (regardless of their GATA3 neoORF status) (p-value=0.246) (FIG. 14).

Table 16 Below Lists Prevalance of GATA3 neoORF

| | Number | Number of patients positive | |
|---|---|---|---|
| | of patients | for GATA3 neoORF | Prevalence |
| all patients | 1,756 | 91 | 5.2% |
| HR + Her2(−) | 1,272 | 77 | 6.1% |
| HR + Her2(−) | 856 | 62 | 7.2% |
| metastatic | | | |

Table 17 Below Lists GATA3 neoORF Specific Peptides and Other Peptides Mapped to Canonical GATA3 Identified from the TCGA Breast Cancer Proteome Dataset

| # | peptide sequence | mapped proteins |
|---|---|---|
| 1 | HGLEPCSMLTGPPAR | GATA3 neoORF |
| 2 | RDGYMFLK | GATA3 neoORF |
| 3 | SSIMKPK | GATA3 neoORF |
| 4 | AGTSCANCQTTTTTLWR | GATA3 |
| 5 | ALGSHHTASPWNLSPFSK | GATA3 |
| 6 | DGTGHYLCNACGLYHK | GATA2, GATA3, GATA4, |
| | | GATA5 |
| 7 | DVSPDPSLSTPGSAGSAR | GATA3 |
| 8 | ECVNCGATSTPLWR | GATA3 |
| 9 | EGIQTR | GATA2, GATA3, GATA4, |
| | | GATA6 |
| 10 | EGIQTRNR | GATA2, GATA3 |
| 11 | KEGIQTR | GATA2, GATA3, GATA4, |
| | | GATA6 |
| 12 | KVHDSLEDFPK | GATA3 |
| 13 | LHNINRPLTMK | GATA3 |
| 14 | LHNINRPLTMKK | GATA3 |
| 15 | MNGQNRPLIKPK | GATA2, GATA3 |
| 16 | NANGDPVCNACGLYYK | GATA2, GATA3 |
| 17 | NSSFNPAALSR | GATA3 |
| 18 | RAGTSCANCQTTTTTLWR | GATA3 |
| 19 | RDGTGHYLCNACGLYHK | GATA2, GATA3, GATA4, |
| | | GATA5 |
| 20 | SSTEGRECVNCGATSTPLWR | GATA3 |
| 21 | VHDSLEDFPK | GATA3 |
| 22 | YQVPLPDSMK | GATA3 |

Investigation of a breast cancer genomic dataset showed that GATA3 neoORF was prevalent in 6-7% of the HR+Her2(−) breast cancer patients depending on their metastatic status. Multiple GATA3 neoORF specific peptides along with other peptides that mapped to canonical GATA3 were identified indicating translation of GATA3 neoORF. Together, these results demonstrated that GATA3 neoORF mutation is prevalent in HR+Her2(−) breast cancer, and when present, the GATA3 neoORF can be translated to yield protein products.

### Example 19 GATA3 neoORF Epitope Count Across HLA Types

This example provides estimation of the typical number of epitopes that could be expected from the GATA3 neoORF across a patient population with diverse HLA types.

### Materials and Methods

All peptides (lengths 8-11) within the common region of the GATA3 neoORF (as defined in EXAMPLE 18) were assessed for presentation probability using an in silico prediction algorithm that jointly considers gene expression, HLA binding potential, and proteasomal processing potential. The algorithm combines the three variables into an overall presentation prediction via a logistic regression fit on mono-allelic mass spectrometry HLA-I profiling data. The following assumptions and tools were used for defining the three input variables:

### Expression

Based on The Cancer Genome Atlas (TCGA) RNA-Seq data, breast cancer samples have a median GATA3 expression of ˜700 transcripts per million (TPM). Assuming that mutant allele and wildtype allele contribute equally to overall GATA3 expression, we estimated that the neoORF transcript would be expressed at 350 TPM.

### HLA Binding Potential

U.S. allele frequencies were imputed based on ethnicity-specific frequencies and assuming that the U.S. population is 62.3% European, 13.3% African American, 6.8% Asian Pacific Islander, and 17.6% Hispanic (Table 18). For the 21 most common HLA-A alleles and the 49 most common HLA-B alleles, peptide binding predictions were ran using the tool NetMHCpan-3.0 (21 and 49 alleles provide 95% population coverage for HLA-A and HLA-B, respectively).

### Processing Potential

A processing potential predictor was trained using publically available mass spectrometry-based HLA-I profiling data, for example, as described in Abelin. J. et al. Immunity, 2017, Bassani-Sternberg, M. et al Molecular & Cellular Proteomics 2015, a neural network configuration that determines processing potential based on the upstream and downstream sequence context of each peptide.

To simulate epitope count per patient, simulant HLA genotypes were created by randomly drawing two HLA-A and two HLA-B alleles (with replacement, to allow for homozygosity) according to their overall U.S. frequencies (Table 18). Most simulant patients had 4 distinct alleles, but because homozygosity was allowed, some simulant patients had just 2 or 3 distinct alleles. For each peptide-allele pair in a simulant patient, a Bernoulli coin flip parameterized by the imputed presentation probability (derived from the above model) was conducted; a positive result was taken to indicate that the given peptide would be presented on the given allele. For each simulant patient, the total number of unique positive peptides was summed to determine the total number of reactive epitopes (meaning that a peptide presented on multiple alleles in the simulation was only counted once). The results were further culled by counting nested epitopes (e.g. a positive 9mer completely contained within a positive 10mer) as a single epitope. Ten thousand patients were simulated in this manner using the statistical programming language R.

### Table 18 Below Lists Allele Frequencies Used in the Simulation

| | | African | Asian Pacific | | USA |
|---|---|---|---|---|---|
| Allele | European | American | Islander | Hispanic | Average |
| A*02:01 | 29.6% | 12.5% | 9.5% | 19.4% | 24.2% |
| A*01:01 | 17.2% | 4.7% | 5.1% | 6.7% | 12.9% |
| A*03:01 | 14.3% | 8.1% | 2.6% | 7.9% | 11.6% |
| A*24:02 | 8.7% | 2.2% | 18.2% | 12.3% | 9.1% |
| A*11:01 | 5.6% | 1.6% | 17.9% | 4.6% | 5.8% |
| A*29:02 | 3.3% | 3.6% | 0.1% | 4.2% | 3.3% |
| A*23:01 | 1.7% | 10.8% | 0.2% | 3.7% | 3.1% |
| A*68:01 | 2.5% | 3.7% | 1.9% | 4.7% | 3.0% |
| A*26:01 | 2.9% | 1.4% | 3.9% | 2.9% | 2.8% |
| A*32:01 | 3.1% | 1.4% | 1.3% | 2.7% | 2.7% |
| A*31:01 | 2.4% | 1.0% | 3.2% | 4.8% | 2.7% |
| A*30:01 | 1.3% | 6.9% | 2.1% | 2.1% | 2.3% |
| A*30:02 | 0.9% | 6.2% | 0.1% | 2.8% | 1.9% |
| A*68:02 | 0.8% | 6.5% | 0.0% | 2.5% | 1.8% |
| A*33:03 | 0.1% | 4.5% | 9.4% | 1.3% | 1.5% |
| A*25:01 | 1.9% | 0.5% | 0.1% | 0.9% | 1.4% |
| A*33:01 | 1.0% | 2.1% | 0.1% | 2.0% | 1.3% |
| A*02:06 | 0.2% | 0.0% | 4.8% | 3.9% | 1.1% |
| A*02:05 | 0.8% | 1.9% | 0.3% | 1.5% | 1.0% |
| A*74:01 | 0.0% | 5.2% | 0.1% | 0.8% | 0.8% |
| A*02:02 | 0.1% | 4.2% | 0.0% | 0.7% | 0.7% |
| B*07:02 | 14.0% | 7.3% | 2.6% | 5.5% | 10.8% |
| B*08:01 | 12.5% | 3.8% | 1.6% | 4.5% | 9.2% |
| B*44:02 | 9.0% | 2.1% | 0.8% | 3.3% | 6.5% |
| B*35:01 | 5.7% | 6.5% | 4.3% | 6.4% | 5.8% |
| B*44:03 | 5.0% | 5.4% | 4.2% | 6.1% | 5.2% |
| B*15:01 | 6.7% | 1.0% | 3.5% | 2.9% | 5.0% |
| B*51:01 | 4.5% | 2.2% | 6.3% | 5.8% | 4.6% |
| B*40:01 | 5.6% | 1.3% | 8.0% | 1.4% | 4.5% |
| B*18:01 | 4.6% | 3.6% | 1.2% | 4.0% | 4.1% |
| B*14:02 | 3.1% | 2.2% | 0.1% | 4.1% | 3.0% |
| B*57:01 | 3.8% | 0.5% | 2.1% | 1.2% | 2.8% |
| B*27:05 | 3.3% | 0.7% | 0.8% | 1.7% | 2.5% |
| B*13:02 | 2.6% | 1.0% | 2.3% | 1.2% | 2.1% |
| B*53:01 | 0.3% | 11.2% | 0.1% | 1.6% | 2.0% |
| B*38:01 | 2.2% | 0.2% | 0.5% | 1.9% | 1.7% |
| B*40:02 | 1.0% | 0.4% | 3.1% | 4.9% | 1.7% |
| B*49:01 | 1.3% | 2.8% | 0.1% | 2.4% | 1.6% |
| B*52:01 | 1.0% | 1.4% | 3.7% | 2.7% | 1.5% |
| B*35:03 | 1.6% | 0.4% | 2.4% | 1.4% | 1.4% |
| B*58:01 | 0.5% | 3.5% | 5.8% | 1.5% | 1.4% |
| B*55:01 | 1.7% | 0.4% | 0.5% | 1.1% | 1.4% |
| B*15:03 | 0.1% | 6.2% | 0.0% | 1.6% | 1.2% |
| B*45:01 | 0.4% | 4.5% | 0.2% | 1.5% | 1.1% |
| B*37:01 | 1.3% | 0.5% | 1.5% | 0.6% | 1.1% |
| B*50:01 | 0.8% | 0.9% | 0.7% | 1.5% | 0.9% |
| B*39:01 | 1.0% | 0.4% | 1.3% | 1.0% | 0.9% |
| B*35:02 | 1.1% | 0.1% | 0.2% | 1.1% | 0.9% |
| B*42:01 | 0.0% | 5.5% | 0.0% | 0.6% | 0.8% |
| B*14:01 | 0.8% | 0.9% | 0.3% | 0.9% | 0.8% |
| B*39:06 | 0.5% | 0.2% | 0.0% | 2.0% | 0.7% |
| B*58:02 | 0.0% | 4.1% | 0.0% | 0.5% | 0.6% |
| B*57:03 | 0.0% | 3.4% | 0.0% | 0.7% | 0.6% |
| B*48:01 | 0.1% | 0.0% | 2.0% | 2.2% | 0.6% |
| B*41:01 | 0.4% | 0.5% | 0.1% | 1.3% | 0.5% |
| B*15:10 | 0.0% | 3.0% | 0.1% | 0.5% | 0.5% |
| B*07:05 | 0.2% | 0.7% | 2.0% | 0.5% | 0.5% |
| B*56:01 | 0.5% | 0.2% | 0.8% | 0.4% | 0.5% |
| B*39:05 | 0.0% | 0.0% | 0.1% | 2.3% | 0.4% |
| B*41:02 | 0.4% | 0.7% | 0.0% | 0.6% | 0.4% |
| B*46:01 | 0.0% | 0.0% | 6.1% | 0.0% | 0.4% |
| B*35:08 | 0.4% | 0.0% | 0.2% | 0.9% | 0.4% |
| B*15:17 | 0.3% | 0.6% | 0.5% | 0.7% | 0.4% |
| B*35:12 | 0.0% | 0.0% | 0.0% | 1.9% | 0.3% |
| B*15:16 | 0.0% | 1.7% | 0.0% | 0.5% | 0.3% |
| B*81:01 | 0.0% | 2.0% | 0.1% | 0.3% | 0.3% |
| B*40:06 | 0.0% | 0.0% | 3.7% | 0.3% | 0.3% |
| B*35:17 | 0.0% | 0.0% | 0.0% | 1.6% | 0.3% |
| B*15:02 | 0.0% | 0.1% | 3.6% | 0.0% | 0.3% |
| B*38:02 | 0.0% | 0.0% | 3.7% | 0.0% | 0.2% |

### Results

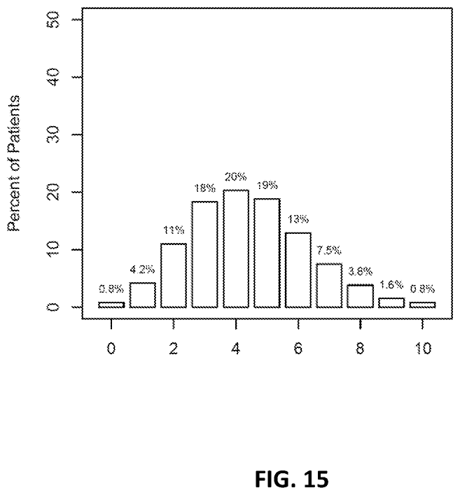

The analysis described in the methods section showed that 95% of patients can present >2 HLA-I epitopes from the GATA3 neoORF (FIG. 15). The GATA3 neoORF can harbor multiple presentable HLA-I epitopes regardless of the HLA genotype of the patient based on details presented above. This shows the effectiveness of a therapy inducing T cell responses against these predicted neoantigens. A subset of the predicted epitopes were selected for validation in follow up studies detailed in Examples 20, 21, 22, 23 below.

### Example 20 Biochemical Measurements of the Epitope

The example below provides biochemical validation of the affinity of epitopes from the GATA3neoORF. A large number of epitopes can bind to many HLA alleles (as described in Example 19). In this example epitopes were evaluated for their ability to bind to several common HLA alleles, namely HLA-A02:01, HLA-1B07:02 and HLA-1B08:01. Both the affinity and stability of the binding between the epitopes and their predicted HLA were evaluated.

The affinity is a measure of the strength of the binding of the epitope to the HLA. Strong binding (generally defined as <500 nM) is an important characteristic for a neoantigen that can be targeted by T cells. This is because the neoantigen must be presented on the surface of tumor cells and therefore must outcompete other antigens produced by the tumor cell for the binding pocket of one of the HLA molecules, as only one peptide can bind to an individual HLA molecule at a time. Further, it has been shown that immunogenic epitopes tend to have strong affinity for their specific HLA.

The stability is a measure of how long a given epitope stays bound to the cognate HLA. Stable binding (generally defined as >1 hour) is also an important characteristic for a neoantigen that can be targeted by T cells. Epitopes must stay bound to tumor cells on the cell surface in order to be recognized by T cells. Further, like affinity, it has been previously shown that immunogenic epitopes tend to bind stably to their specific HLA.

In order to evaluate the stability and affinity of epitopes for their cognate HLA molecules, peptides were synthesized at purities >70% (by UV analysis of % Area) and diluted to 20 mM or less and their affinities and stabilities were measured.

In this example, the affinity and stability of the binding between 14 epitopes and cognate HLA molecules is reported. Four epitopes were studied on HLA-A02:01, five epitopes were studied on HLA-B07:02, and eight epitopes were studied on HLA-B08:01 (three epitopes were studied on both HLA-B07:02 and HLA-B08:01). All peptides demonstrated strong binding by affinity, ranging from 9.5 nM to 242.8 nM. Stabilities ranged from 0 hours to 21.7 hours, with at least one epitope on each allele exceeding 1 hour. These results show that there is at least one strong epitope derived from the GATA3 neoORF per allele.

### Materials and Methods

### Selection of Epitopes for Biochemical Measurements

Multiple epitopes derived from the GATA3 neoORF were selected for confirmation of their ability to bind to a specific common HLA allele, namely HLA-A02:01, HLA-B07:02, or HLA-B08:01. These epitopes were predicted to range from weak to strong binders.

### Solid Phase Peptide Synthesis

Peptides were made on 5 μmol scale using solid phase peptide synthesis on the Intavis Peptide Synthesizer. Fmoc deprotections were performed using 20% piperidine in DMF and rinsed with neat DMF. All amino acids were double coupled at 15 minute durations at room temperature using 60 μL of 0.5M amino acid (6eq), 55 μL 0.5 M HCTU (5.5eq), 5 μL NMP (0.5eq) and 14 μL 4 M NMM (11.2eq). After each double coupling cycle, acetyl capping was performed by adding 100 μL of a DIEA solution (made first as a 2M solution in NMP and then diluted to 12.5% using DMF) and 6.25% acetic anhydride in DMF for 15 minutes before vacuum draining and rinsing with DMF. The deprotection, wash, double coupling, acetyl capping, wash cycle was repeated for each amino acid in the sequence. Final deprotection was performed with 20% piperidine in DMF and final washes with DMF, EtOH, and DCM. A final drain dry was completed for 5 minutes on the instrument after which plate bottoms were rinsed with DCM.

### Cleavage of Peptides

The peptides were cleaved using a solution of 92.5% TFA, 2.5% TIPS, 2.5% H₂O, 2.5% EDT. After 1 hour the plates were vacuum drained into 1.2 mL Micronic racks. After a total of 3 hours the peptides were then precipitated with cold diethyl ether via centrifugation.

### UPLC-UV-MS Analysis of Peptides

Crude peptides were dried and re-suspended in 1:1 ACN:H₂O containing 0.1% TFA and kept at −80° C. until completely frozen. Peptides were then freeze-dried to isolate the peptide in powder form. Peptide powders were dissolved first in neat DMSO and then diluted 3:1 in DMSO:H₂O for UPLC-UV-MS analysis. UV monitoring was performed at a wavelength of 214 nm with the mass detector range spanning 200-1250 Da. The UPLC-UV-MS method used for peptides less than 9 amino acids in length comprised a gradient of 0-100% mobile phase B (0.085% TFA in acetonitrile, with a corresponding mobile phase A of 0.1% TFA in water) over 5 minutes on a 2.1×50 mm 1.7 μM BEH Acquity UPLC column, while the method for peptides greater than 9 amino acids comprised a gradient of 10-80% mobile phase B over 8 minutes on a 2.1×100 mm 1.7 μM BEH Acquity UPLC column.

### Determination of Peptide Concentration by A214 Method

Crude peptides were dissolved in neat DMSO with concentrations of 2-5 mg/mL for evaluation by the A214 method. The peptide peak area of a UPLC-UV chromatogram is proportional to the amount of peptide injected for analysis and the extinction coefficient of the peptide at the detection wavelength. Therefore, the concentration of a peptide sample can be determined by comparing its UV peak area with the UV peak area of a reference peptide of known concentration and considering the respective extinction coefficients. The following equation is used to calculate the peptide concentration:

C=Cref*(Asam*Eref*Vref)/(Aref*Esam*Vsam) Equation 7.

where, C is the peptide sample concentration in mM, C_{ref} is the reference peptide concentration in mM, Aₛₐₘ is the UV peak area of peptide sample, A_{ref} is the UV peak area of reference peptide, E_{ref} is the extinction coefficient of reference peptide in M⁻¹ cm⁻¹, Eₛₐₘ is the extinction coefficient of peptide sample in M⁻¹ cm⁻¹, Vₛₐₘ is the injection volume of sample, and V_{ref} is the injection volume of reference peptide.

The extinction coefficient of a peptide at 214 nm is predicted by combining the extinction coefficients of individual amino acids and peptide bonds. A reference peptide with sequence of RAKFKQLL (peptide ID LS-18) at 0.2 mg/mL is run in sequence with the crude peptide samples on the UPLC-UV-MS. The UV peak areas and the calculated extinction coefficients are then used to calculate the peptide concentration in mM.

### Affinity Measurements

The binding affinity of a peptide to HLA molecules was measured by assessing its ability to outcompete a defined radiolabeled peptide for the binding pocket on the HLA molecule. This was done by purifying HLA molecules and incubating them with multiple concentrations of the peptide of interest and a high-affinity binding peptide that is radiolabeled. After 2 days of incubation, unbound radiolabeled peptide was separated by size-exclusion gel filtration chromatography, and the fraction of HLA molecules that have the radiolabeled peptide was determined. Peptides that have low percentages of bound radiolabeled peptide at the end of the assay have a strong affinity for the HLA. Quantitatively, the concentration of the peptide of interest required to inhibit the binding of the radiolabeled peptide by 50% can be determined by a regression analysis of the inhibition across multiple concentrations. This IC50 measurement was used as an approximation of the true binding affinity.

In the first wave of peptides analyzed, the actual concentrations of the peptides were known based on A214 measurements and any necessary corrections based on concentration were done. In the second wave of peptides analyzed, the concentration of all peptides were presumed to be 20 mM and initial IC50s were calculated based on that presumption, with adjustments accounting for actual concentration later performed. For peptides with actual concentration below 20 mM, the measured IC50 was corrected by multiplying by the actual concentration as determined by the A214 method and dividing by 20 mM.

### Stability Measurements

To measure the binding stability of peptides to Class I MHC, synthetic genes encoding biotinylated MHC-I heavy and light chains are expressed in E. coli and purified from inclusion bodies using standard methods. The light chain (β2m) was radio-labeled with iodine (125I), and combined with the purified MHC-I heavy chain and peptide of interest at 18° C. to initiate pMHC-I complex formation. These reactions were carried out in streptavidin coated microplates to bind the biotinylated MHC-I heavy chains to the surface and allow measurement of radiolabeled light chain to monitor complex formation. Dissociation was initiated by addition of higher concentrations of unlabeled light-chain and incubation at 37° C. Stability was defined as the length of time in hours it takes for half of the complexes to dissociate, as measured by scintillation counts. Duplicate measurements were performed. The average of the two measurements was taken as the stability.

### Results

### Affinity Measurements

### Table 19 Below Lists Epitope Affinity Measurements.

| | | | Actual | | |
|---|---|---|---|---|---|
| | | | Peptide | Meas- | Cor- |
| | | | Concen- | ured | rected |
| | Peptide | HLA | tration | IC50 | IC50 |
| Wave | Sequence | Allele | (mM) | (nM) | (nM) |
| 2 | MLTGPPARV | A02:01 | 13.7 | 15.4 | 10.6 |
| 1 | SMLTGPPARV | A02:01 | 20.0 | 15.4 | 15.4 |
| 2 | TLQRSSLWCL | A02:01 | 8.4 | 281.2 | 117.7 |
| 1 | YMFLKAESKI | A02:01 | 20.0 | 165.9 | 165.9 |
| 2 | FATLQRSSL | B07:02 | 20.0 | 14.0 | 14.0 |
| 2 | KPKRDGYMF | B07:02 | 20.0 | 28.2 | 28.2 |
| 2 | KPKRDGYMFL | B07:02 | 17.0 | 115.2 | 98.1 |
| 2 | GPPARVPAV | B07:02 | 15.7 | 281.9 | 221.2 |
| 2 | MFATLQRSSL | B07:02 | 15.2 | 350.3 | 266.9 |
| 2 | ESKIMFATL | B08:01 | 15.2 | 23.3 | 17.7 |
| 2 | FLKAESKIM | B08:01 | 20.0 | 21.9 | 21.9 |
| 2 | FATLQRSSL | B08:01 | 19.4 | 27.5 | 26.6 |
| 1 | YMFLKAESKI | B08:01 | 20.0 | 32.0 | 32.0 |
| 2 | IMKPKRDGYM | B08:01 | 16.5 | 40.2 | 33.2 |
| 2 | MFATLQRSSL | B08:01 | 20.0 | 53.4 | 53.4 |
| 2 | FLKAESKIMF | B08:01 | 18.3 | 90.0 | 82.3 |
| 2 | LHFCRSSIM | B08:01 | 14.2 | 167.3 | 118.7 |

*Note that actual peptide concentration and measured IC50 reported here are rounded from the raw data, but corrected IC50 values were calculated using the un-rounded raw data.

### Stability Measurements

### Table 20 Below Lists Stability Measurements

| | | | Experiment | Experiment | Average |
|---|---|---|---|---|---|
| | | | 1 | 2 | half- |
| | | HLA | half-life | half-life | life |
| Wave | Sequence | Allele | (hours) | (Hours) | (Hours) |
| 2 | TLQRSSLWCL | A02.01 | 0.5 | 0.5 | 0.5 |
| 2 | MLTGPPARV | A02.01 | 5.8 | 5.8 | 5.8 |
| 1 | YMFLKAESKI | A02:01 | 0.6 | 0.6 | 0.6 |
| 1 | SMLTGPPARV | A02:01 | 21.7 | 21.8 | 21.7 |
| 2 | KPKRDGYMFL | B07.02 | 3.3 | 3.3 | 3.3 |
| 2 | KPKRDGYMF | B07.02 | 8.3 | 9.0 | 8.6 |
| 2 | FATLQRSSL | B07.02 | 0.7 | 0.6 | 0.7 |
| 2 | MFATLQRSSL | B07.02 | 0.0 | 0.0 | 0.0 |
| 2 | GPPARVPAV | B07.02 | 1.5 | 1.8 | 1.6 |
| 2 | FLKAESKIMF | B08.01 | 0.0 | 0.0 | 0.0 |
| 2 | FATLQRSSL | B08.01 | 0.0 | 0.0 | 0.0 |
| 2 | MFATLQRSSL | B08.01 | 0.0 | 0.0 | 0.0 |
| 2 | ESKIMFATL | B08.01 | 1.0 | 1.6 | 1.3 |
| 2 | FLKAESKIM | B08.01 | 0.8 | 1.5 | 1.2 |
| 2 | LHFCRSSIM | B08.01 | 0.0 | 0.0 | 0.0 |
| 2 | IMKPKRDGYM | B08.01 | 0.4 | 0.4 | 0.4 |
| 1 | YMFLKAESKI | B08:01 | 0.4 | 0.5 | 0.4 |

In Example 20, predicted epitopes from the GATA3 neoORF on multiple common HLA molecules (HLA-A02:01, HLA-B07:02, HLA-B08:01) were evaluated. All epitopes were determined to have a strong affinity (<500 nM). A subset of these epitopes were also stable binders (>1 hour), with at least one strong binder on each of the HLA alleles evaluated. These data show that GATA3 neoORF epitopes are present across multiple HLA alleles.

### Example 21: Generation of Cell Line with GATA3 Mutation and HLA Allele

This Example described preparation of a cell line with the GATA binding protein 3 (GATA3) novel open reading frame (neoORF) mutation and high-prevalence HLA alleles, HLA-A02:01 and HLA-B07:02. This cell line can be used as an in vitro surrogate of tumor cells that contain GATA3 neoORF mutations. Cell lines that naturally harbor the specific GATA3 neoORF of focus are not readily available, one was prepared by stable lentiviral transduction of a commonly used cell line, HEK293T. This cell line was chosen because it naturally expresses two of the common HLA alleles, HLA-A02:01 and HLA-B07:02. This modified cell line was used for functional assays with T cells (Example 25 and Example 26). Additionally, this cell line was used for validation of neoantigens processing/presentation from the GATA3 neoORF on multiple HLA alleles (Example 22). For these studies, the HLA alleles were transiently transfected into the modified cell line.

### Materials and Methods

### Overview of Generation of GATA3 Mutation Cell Line

The generation of GATA3 mutation transduced HEK 293T cell entailed (a) GATA3 mutation encoded plasmid design, production of lenti-virus, transduction of GATA3 mutation to HEK 293T cell line, and selection of transduced cells. These steps for generation of GATA3 mutation cell line are described below.

### Cell Lines and Culture

HEK 293T cell lines were purchased from the American Type Culture Collection (Rockford, Md., USA) and maintained in DMEM 10% FBS, and Pen/Strep medium.

### GATA3 Mutation Encoded Plasmid Design

### GATA3 Mutation Gene

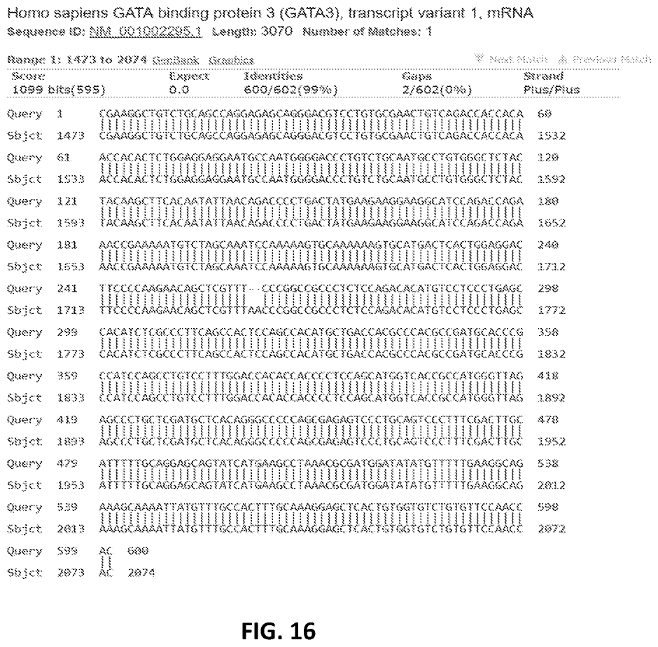

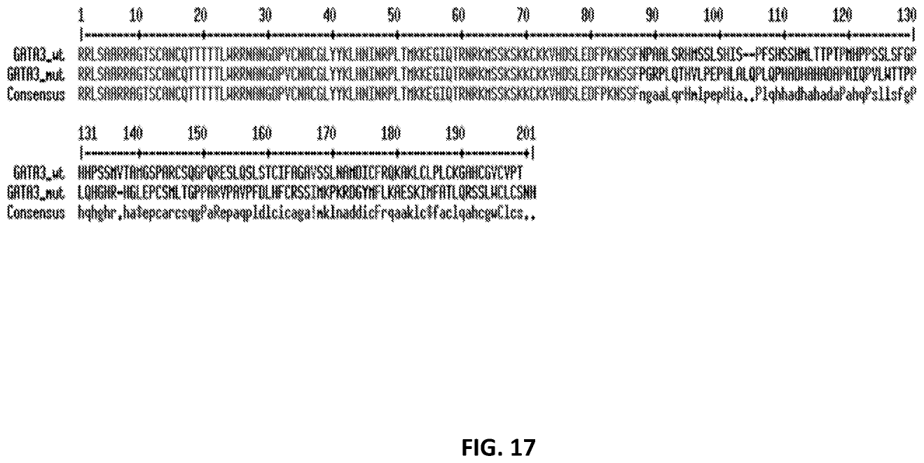

For the efficient expression of GATA3 mutation gene plasmid construct, 600 bp GATA binding protein3 (GATA3) wild-type sequence from 1473 to 2074 (which contains coding DNA sequence (CDS) sequence from 558 to 1892) was obtained from NCBI Reference Sequence. NM_001002295.01. GATA3 mutation sequence was further generated by deleting 2 nucleotides at 1734 and 1735 from reference sequence (FIG. 16). GATA3 mutation sequence then translates a frameshift at position 87 of amino acid sequence from wild-type sequence (FIG. 17). This DNA construct can cover 87 residues of wild type GATA3 amino acid sequence and 114 of the frameshifted GATA3 neoORF amino acid sequence which is caused by the deletion.

### GATA3 Mutation Plasmid Design

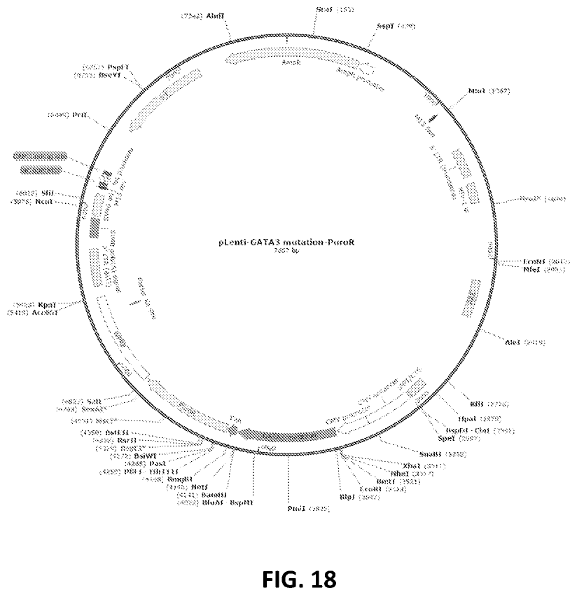

GATA3 mutation sequences were codon-optimized, synthesized and cloned into pCDH-CMV-Puro vector (Genescript) (FIG. 18).

### Lenti-Virus Production

Lenti-X 293T cells (ClonTech) were cultured in complete culture media (DMEM containing 10% FBS, Pen/Strep) and transfected with GATA3 mutation encoded lentiviral plasmid to produce lentivirus for GATA3 mutation gene. The day before the transfection, 8×10⁵ of the cells were plated per well of a 6 well plate. The culture media was replaced at the day of transfection. 4 μg of lentiviral construct plasmid and 4.6 μL of the lentiviral packaging plasmid mix (Sigma-Aldrich) were mixed in Opti-MEM (Thermo Fisher). The mixture was mixed with 10 μL of FuGENE HD (Promega) and added to the cells directly. At 24 hours later, the media was replaced with the fresh complete culture media. The supernatant contained lentivirus was harvested at 72 hours after transfection.

### Transduction of GATA3 Mutation

5×10⁵ of HEK 293T cells (ATCC) were plated in 2 mL of DMEM media contained 6 μg/mL polybrene and 10% FBS on 12-well plate. 130 μL of supernatant containing GATA3 lentivirus were added to cells directly. The cells were incubated at 5% CO₂ incubator. The media was replaced with DMEM media with 10% FBS and Pen/Strep at 24 hours.

### Puromycin Selection

1 μg/mL concentration of puromycin treatment started at day 2 after transduction of GATA3 mutation lenti-virus. The cells were cultured and expanded with DMEM media with 10% FBS, Pen/Strep and 1 μg/mL of Puromycin until harvest.

Transfection with HLA-Encoding Constructs

1.5×10⁷ of GATA3 mutation transduced HEK 293T cells were seeded in T175 flask. 15 μg of HLA-A02:01, HLA-B07:02 or HLA-B08.01 encoded plasmids (Genewiz) were mixed with 70 μL of Fugene HD (Promega) and incubated at room temperature for 15 minutes. The mixtures of each HLA type plasmids and Fugene HD were added to GATA3 mutation transduced HEK 293T cells in T175 flask for transfection. The 3 different HLA transfected and GATA3 mutation transduced HEK 293T cells were cultured for 48 hours before harvest.

### Harvest GATA3 Mutation Transduced and HLA Transfected Cells

The cells were washed with 1×PBS and added 0.25% Trypsin-EDTA (Thermo-Fisher scientific). After 3 minutes of incubation at 37° C., the cells were resuspended and harvested with DMEM media with 10% FBS and Pen/Strep. Washing steps were performed 3 times which includes centrifugation at 1,500 rpm for 5 min followed by suspension with PBS buffer. The cell pellets were snap frozen on dry-ice in 70% ethanol. The frozen cell pellets were stored at −80° C. freezer for proteomics analysis.

### Results

GATA3 mutation transduced HEK 293T were used as target cells to evaluate a GATA3 specific TCR functional assay, and as material to evaluate GATA mutation presentation on HLA-A02.01 by mass-spectrometry (Example 22). Outlined below are results demonstrating generation of GATA3 mutation expressed HEK 293T cells

### GATA3 Mutation Plasmid Construct

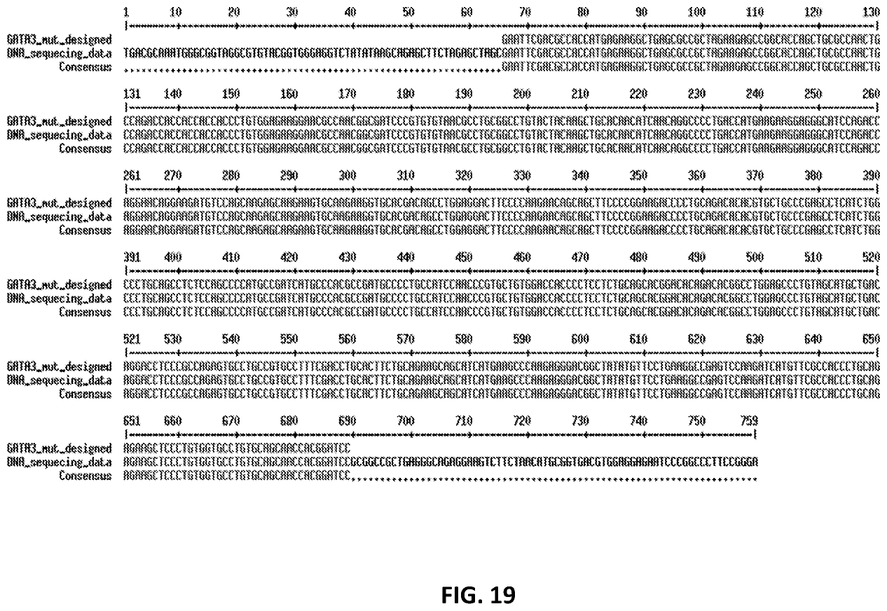

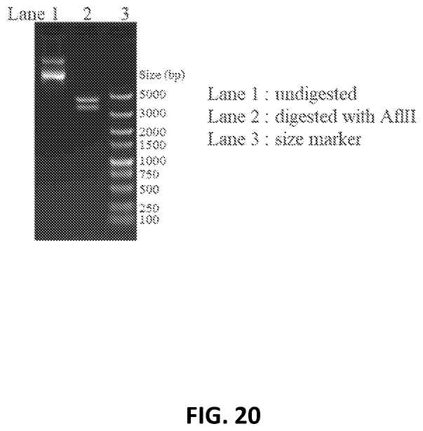

The GATA3 mutation encoded plasmid construct was generated and evaluated by DNA sequencing at GENEWIZ. DNA sequencing data of final GATA3 mutation encoded plasmid is 100% matched with GATA3 mutation gene sequence designed (FIG. 19). After the restriction enzyme AfIII digestion, two DNA bands were observed between 5000 bp and 3000 bp in lane 2 of a gel electrophoresis assay. These bands correlate with the expected sizes of 4243 bp and 3424 bp, respectively (FIG. 20).

### GATA3 Mutation Transduction and Harvest

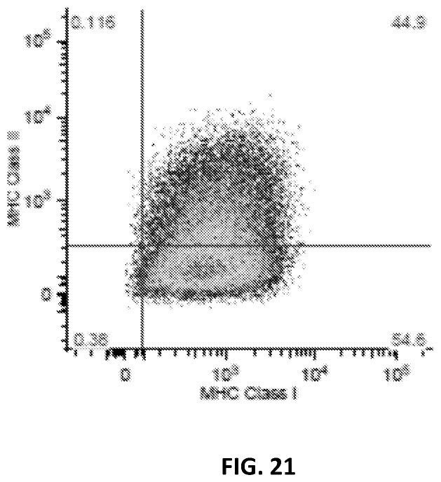

HEK 293T cells were used for GATA3 mutation transduction. The transduced cells were cultured until reached 200×10⁶ cells of total cell number. At the harvest date, 1×10⁶ cells were used for HLA-Class I and HLA-Class II expression by Flow cytometer (FIG. 21). 99.5% cells were HLA-ClassI positive. 193×10⁶ cells were frozen for proteomics analysis.

### HLA Transfection

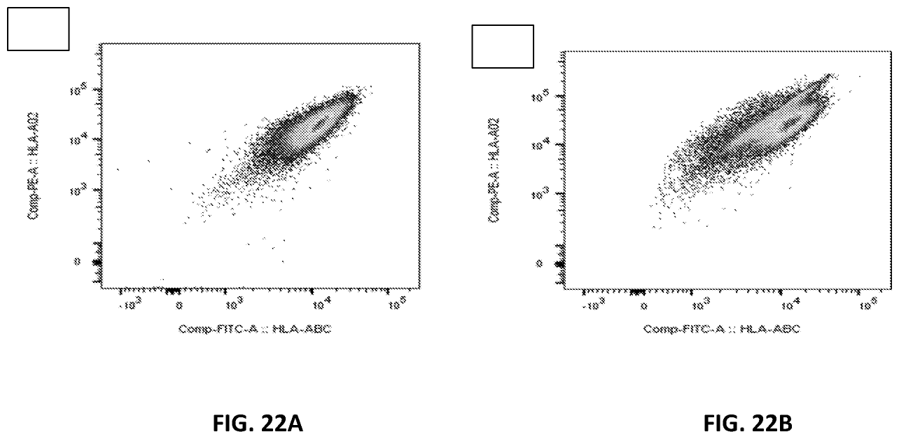

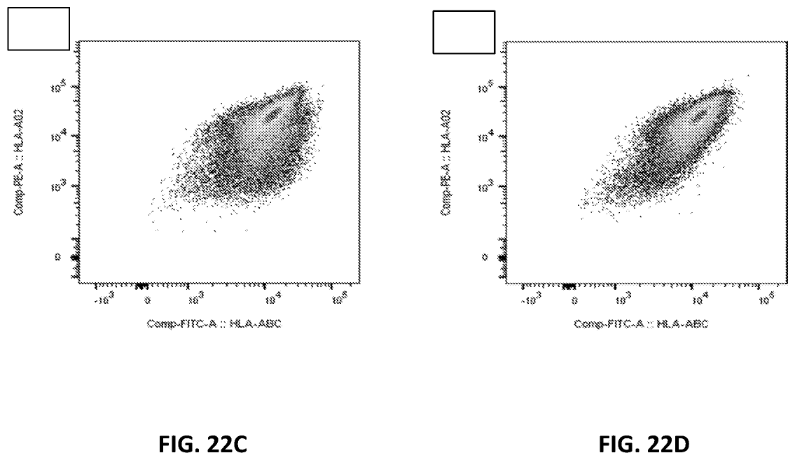

GATA3 mutation transduced HEK 293T cells were transiently transfected with BAP tagged HLA-A02.01, BAP tagged HLA-B07.02, and BAP tagged HLA-B08.01 encoded expression plasmid. The transfected cells were cultured for 48 hours and harvested. At the harvest date, 1×10⁶ cells were used for HLA-A02.01 and HLA-Class I expression by Flow cytometry (FIG. 22). Non-transfected (FIG. 22A), HLA-A02.01 transfected (FIG. 22B), HLA-B07.02 transfected (FIG. 22C) and HLA-B08.01 transfected (FIG. 22C) GATA3 HEK293T cells. All transfected cells highly expressed HLA-A02.01 and HLA-Class I.

A modified cell line was generated that expresses the GATA3 neoORF by stable transduction of lentivirus containing the mutated GATA3 gene into HEK293T cells. The GATA3 mutation transduced HEK 293T cells expressed HLA-Class I. This cell line was subsequently used as a target for functional assays with T cells specific for GATA3 neoantigens (Example 25 and Example 26). Further, after transfection with several common HLA alleles, these cell lines showed wider distribution of HLA-Class I and HLA-A:02 expression level and were used for evaluation of processing and presentation of multiple neoantigens on these alleles (Example 22).

### Example 22 Validation of GATA3 neoORF Peptide Epitopes by Mass Spectrometry

This Example provides validation of the endogenous processing and presentation of predicted peptide epitopes derived from the common region of the GATA binding protein 3 (GATA3) novel open reading frame (neoORF) for binding to HLA-A*02:01, HLA-B*07:02, and HLA-B*08:01 heterodimers by mass spectrometry. HEK293T cells were engineered to stably express GATA3 neoORF and transiently transfected to express biotin acceptor peptide (BAP)-tagged HLA alleles of interest. Prediction of allele-specific peptide epitopes and the generation of HEK293T cells are described in Example 19 and Example 21, respectively. HLA-peptide complexes were isolated from cellular lysates by affinity pull-down of the biotinylated BAP-tag expressed on the alpha chain of each HLA class I heterodimer. Peptide ligands were released from HLA-peptide complexes by treatment with acid and desalted by reverse phase liquid chromatography. HLA-peptide ligands were further separated by nano liquid chromatography coupled to a high-resolution tandem mass spectrometer (nLC-MS/MS). Predicted peptide epitopes derived from the GATA3 neoORF were subjected to targeted nLC-MS/MS whereby a priori knowledge of each peptide epitope's precursor mass was used to select each peptide epitope's theoretical monoisotopic mass for fragmentation by higher-energy collisional dissociation (HCD) and subsequent peptide sequencing. GATA3 neoORF peptide epitopes were matched to resulting tandem mass spectra (MS/MS) by a database matching algorithm against a database containing the GATA3 neoORF, and by spectral comparison of precursor mass (MS) and MS/MS spectra corresponding to their synthetic peptide counterparts.

In total, five peptide epitopes from the common region of the GATA3 neoORF that bound to three different HLA heterodimers were detected by nLC-MS/MS in engineered HEK293T cells. For HLA-A*02:01, the following two of four targeted peptide epitopes were detected: SMLTGPPARV and MLTGPPARV. For HLA-B*07:02, the following two of five targeted peptide epitopes were detected: KPKRDGYMF and KPKRDGYMFL. For HLA-B*08:01, the following one of eight targeted peptide epitopes was detected: ESKIMFATL. The detection and identification of these peptide epitopes by nLC-MS/MS from cells expressing the GATA3 neoORF demonstrated that they are endogenously processed and subsequently bound by HLA heterodimers.

### Materials and Methods

Peptides: ¹²C4N synthetic peptides corresponding to GATA3 neoORF peptide epitopes were synthesized.

Table 21 Below Provides List of Synthetic Peptides Corresponding to GATA3 neoORF Predicted Peptide Epitopes.

| | | | Theoretical |
|---|---|---|---|
| | | | Molecular |
| Allele | Sequence | Length | Weight |
| HLA-A*02:01 | SMLTGPPARV | 10 | 1027.5484 |
| HLA-A*02:01 | MLTGPPARV | 9 | 940.5164 |
| HLA-B*07:02 | KPKRDGYMF | 9 | 1140.5750 |
| HLA-B*07:02 | KPKRDGYMFL | 10 | 1253.6590 |
| HLA-B*08:01 | ESKIMFATL | 9 | 1054.5368 |

### Cell Culture

Generation of the engineered HEK293T cells that stably express the GATA3 neoORF and the transient transfection of each affinity-tagged (BAP-tagged) allele was described in Example 21. Table 22 lists the samples and cell numbers that were used for targeted nLC-MS/MS.

Table 22 Below Provides Summary of Samples for Targeted nLC-MS/MS

| | | Cell Number | |
|---|---|---|---|
| Allele | Cell Type | (×10⁶) | Pull-Down Type |
| HLA-A*02:01 | HEK293T | 55 | Affinity-tag |
| HLA-B*07:02 | HEK293T | 61 | Affinity-tag |
| HLA-B*08:01 | HEK293T | 60 | Affinity-tag |

### Pull-Down of Affinity-Tagged (BAP-Tagged) HLA-Peptide Complexes

Frozen cell pellets containing BAP-tagged HLA molecules were thawed on ice for 20 min then gently lysed by hand pipetting in cold lysis buffer [20 mM Tris-Cl pH 8, 100 mM NaCl, 6 mM MgCl₂, 1.5% (v/v) Triton X-100, 60 mM octyl B-D-glucopyranoside, 0.2 mM of 2-Iodoacetamide, 1 mM EDTA pH 8, 1 mM PMSF, 1× cOmplete EDTA-free protease inhibitor cocktail] at a ratio of 1.2 mL lysis buffer per 50×10⁶ cells. Lysates were incubated end/over/end at 4° C. for 15 min with Benzonase nuclease at a ratio of >250 units Benzonase per 50×10⁶ cells to degrade DNA/RNA, then centrifuged at 15,000×g at 4° C. for 20 min to remove cellular debris and insoluble materials. Cleared supernatants were transferred to new tubes and BAP-tagged HLA molecules were biotinylated by incubating end/over/end at room temperature for 10 min in a 1.5 mL tube with 0.56 μM biotin, 1 mM ATP/1 mM magnesium acetate, and 3 μM BirA. The supernatants were incubated end/over/end at 4° C. for 30 min with a volume corresponding to 200 μL of Pierce high-capacity NeutrAvidin beaded agarose resin slurry per 50×10⁶ cells to affinity-enrich biotinylated-HLA-peptide complexes. Finally, the HLA-bound resin was washed four times with 1 mL of cold wash buffer (20 mM Tris-Cl pH 8, 100 mM NaCl, 60 mM octyl B-D-glucopyranoside, 0.2 mM of 2-Iodoacetamide, 1 mM EDTA pH 8), then washed four times with 1 mL of cold 10 mM Tris-Cl pH 8. Between washes, the HLA-bound resin was gently mixed by hand then pelleted by centrifugation at 1,500×g at 4° C. for 1 min. The NeutrAvidin beaded agarose resin was washed three times with 1 mL cold PBS before use. The washed HLA-bound resin was stored at −80° C. for less than one week prior to HLA-peptide elution.

### HLA-Peptide Desalting, Reduction, and Alkylation

HLA-peptides were eluted from affinity-tagged (BAP-tagged) HLA complexes and simultaneously desalted using a Sep-Pak solid-phase extraction system. In brief, Sep-Pak cartridges were attached to a 24-position solid phase extraction manifold, activated two times with 200 μL of methanol followed by 100 μL of 50% (v/v) acetonitrile/0.1% (v/v) formic acid, then washed four times with 500 μL of 1% (v/v) formic acid. To dissociate HLA-peptides from affinity-tagged (BAP-tagged) HLA molecules and facilitate peptide binding to the tC18 solid-phase, 400 μL of 3% (v/v) acetonitrile/5% (v/v) formic acid was added to the tubes containing HLA-bound beaded agarose resin. The slurry was mixed by pipetting, then transferred to the Sep-Pak cartridges. The tubes and pipette tips were rinsed with 1% (v/v) of formic acid (2×200 μL) and the rinsate was transferred to the cartridges. 100 femtomole of Pierce peptide retention time calibration mixture was added to the cartridges as a loading control. The beaded agarose resin was incubated two times for 5 min with 200 μL of 10% (v/v) acetic acid to further dissociate HLA-peptides from the affinity-tagged (BAP-tagged) HLA molecules, then washed four times with 500 μL of 1% (v/v) formic acid. HLA-peptides were eluted off the tC18 into new 1.5 mL micro tubes by step fractionating with 250 μL of 15% (v/v) acetonitrile/1% (v/v) formic acid followed by 250 μL of 30% (v/v) acetonitrile/1% (v/v) formic acid. The solutions used for activation, sample loading, washing, and elution flowed via gravity, but vacuum (≥−2.5 PSI) was used to remove the remaining eluate from the cartridges. Eluates containing HLA-peptides were frozen, dried via vacuum centrifugation, and stored at −80° C. before being subjected to reduction, alkylation, and a second desalting workflow.

Reduction and alkylation of cysteine-containing HLA-peptides was performed in 1.5 mL micro tubes as follows. Dried peptides were solubilized in 200 μL of 10 mM Tris-Cl pH 8, then reduced by incubating with 5 mM of dithiothreitol at 60° C. for 30 min while shaking at 1,000 rpm in a ThermoMixer. Reduced thiols were alkylated by incubating with 15 mM 2-Iodoacetamide at room temperature for 30 min in the dark. Any unreacted 2-Iodoacetamide was quenched by incubating with 5 mM of Dithiothreitol for 15 min at room temperature in the dark. Samples were desalted immediately after reduction and alkylation.

Secondary desalting of the HLA-peptide samples was performed with in-house built StageTips packed using two 16-gauge punches of an Empore C18 solid phase extraction disk. StageTips were activated two times with 100 μL of methanol followed by 50 μL of 99.9% (v/v) acetonitrile/0.1% (v/v) formic acid, then washed three times with 100 μL of 1% (v/v) formic acid. The peptide solution was acidified by adding 200 μL of 3% (v/v) acetonitrile/5% (v/v) then and loaded onto StageTips. The tubes and pipette tips were rinsed with 200 μL of 3% (v/v) acetonitrile/5% (v/v) followed by 1% (v/v) formic acid (2×100 μL) and the rinse volume was transferred to the StageTips. StageTips were washed five times with 100 μL of 1% (v/v) formic acid. Peptides were eluted into 1.5 mL micro tubes using a step gradient of 20 μL 15% (v/v) acetonitrile/1% (v/v) formic acid followed by two 20 μL cuts of 30% (v/v) acetonitrile/1% (v/v) formic acid. Sample loading, washes, and elution were performed on a tabletop centrifuge with a maximum speed of 1,800-3,500×g at room temperature. Eluates were frozen, dried via vacuum centrifugation, and stored at −80° C.

HLA-Peptide Sequencing by nLC-MS/MS

All nLC-MS/MS analyses employed the same liquid chromatography separation conditions described below. Peptides were chromatographically separated using an EASY-nLC 1200 System fitted with a PicoFrit 75 μm inner diameter and 10 μm emitter nanospray column packed at −1,000 psi of pressure with helium to ˜35 cm with ReproSil-Pur 120A C18-AQ 1.9 μm packing material and heated at 60° C. during separation. The column was equilibrated with 10× bed volume of solvent A [3% (v/v) acetonitrile/0.1% (v/v) formic acid], samples were loaded in 4 μL 3% (v/v) acetonitrile/5% (v/v) formic acid, and peptides were eluted with a linear gradient of 6-40% Solvent B [80% (v/v) acetonitrile/0.1% (v/v) formic acid] over 84 min, 40-60% Solvent B over 9 min, then held at 90% Solvent B for 5 min and 50% Solvent B for 9 min to wash the column. Linear gradients for were run at a rate of 200 nL/min.

Peptides were eluted into an Orbitrap Fusion Lumos Tribrid Mass Spectrometer equipped with a Nanospray Flex Ion source at 2.5 kV. A full-scan MS was acquired at a resolution of 15,000 from 300-1,800 m/z with an automatic gain control (AGC) target of 4×10⁵ and 50 millisecond max injection time. Each MS scan was followed by MS/MS scans according to an inclusion mass list (Table 23) comprising the calculated ion masses (m/z) of the targeted GATA3 neoORF peptide epitopes and their predicted charge states (z). Additional calculated ion masses were included for peptides that met the following criteria: i) multiple charge states expected due to presence of one or more basic residues in the sequence and/or ii) peptides containing amino acids that were expected to be modified during sample processing such as cysteine, methionine, and N-terminal glutamine. Maximum injection times varied between 100 milliseconds and 120 milliseconds to maintain cycle times of 2-2.8 sec across the chromatographic peak. MS/MS scans were acquired at a resolution of 15,000 from 110 to 1,300-1,500 m/z, using an isolation width of 1 m/z, normalized HCD collision energy of 34, and an AGC target of 1×10⁵.

| | | Peptide | | |
|---|---|---|---|---|
| # | Allele | Sequence* | m/z | z |
| 1 | HLA-A*02:01 | MLTGPPARV | 471.2655 | 2 |
| 2 | HLA-A*02:01 | mLTGPPARV | 479.2629 | 2 |
| 3 | HLA-A*02:01 | SMLTGPPARV | 514.7815 | 2 |
| 4 | HLA-A*02:01 | SmLTGPPARV | 522.7790 | 2 |
| 5 | HLA-A*02:01 | TLQRSSLWCamcL | 421.8887 | 3 |
| 6 | HLA-A*02:01 | TLQRSSLWCamcL | 632.3294 | 2 |
| 7 | HLA-A*02:01 | TLQRSSLWCcysL | 442.5495 | 3 |
| 8 | HLA-A*02:01 | TLQRSSLWCcysL | 663.3207 | 2 |
| 9 | HLA-A*02:01 | TLQRSSLWCL | 402.8815 | 3 |
| 10 | HLA-A*02:01 | TLQRSSLWCL | 603.8186 | 2 |
| 11 | HLA-A*02:01 | YMFLKAESKI | 410.5581 | 3 |
| 12 | HLA-A*02:01 | YmFLKAESKI | 415.8898 | 3 |
| 13 | HLA-A*02:01 | YMFLKAESKI | 615.3336 | 2 |
| 14 | HLA-A*02:01 | YmFLKAESKI | 623.3310 | 2 |
| 1 | HLA-B*07:02 | FATLQRSSL | 511.7851 | 2 |
| 2 | HLA-B*07:02 | GPPARVPAV | 432.2585 | 2 |
| 3 | HLA-B*07:02 | KPKRDGYMF | 381.1989 | 3 |
| 4 | HLA-B*07:02 | KPKRDGYmF | 386.5306 | 3 |
| 5 | HLA-B*07:02 | KPKRDGYMF | 571.2948 | 2 |
| 6 | HLA-B*07:02 | KPKRDGYmF | 579.2922 | 2 |
| 7 | HLA-B*07:02 | KPKRDGYMFL | 418.8936 | 3 |
| 8 | HLA-B*07:02 | KPKRDGYmFL | 424.2253 | 3 |
| 9 | HLA-B*07:02 | KPKRDGYMFL | 627.8368 | 2 |
| 10 | HLA-B*07:02 | KPKRDGYmFL | 635.8343 | 2 |
| 11 | HLA-B*07:02 | MFATLQRSSL | 577.3053 | 2 |
| 12 | HLA-B*07:02 | mFATLQRSSL | 585.3028 | 2 |
| 1 | HLA-B*08:01 | ESKIMFATL | 520.2783 | 2 |
| 2 | HLA-B*08:01 | ESKImFATL | 528.2757 | 2 |
| 3 | HLA-B*08:01 | FATLQRSSL | 511.7851 | 2 |
| 4 | HLA-B*08:01 | FLKAESKIM | 356.2037 | 3 |
| 5 | HLA-B*08:01 | FLKAESKIm | 361.5353 | 3 |
| 6 | HLA-B*08:01 | FLKAESKIM | 533.8019 | 2 |
| 7 | HLA-B*08:01 | FLKAESKIm | 541.7994 | 2 |
| 8 | HLA-B*08:01 | FLKAESKIMF | 405.2265 | 3 |
| 9 | HLA-B*08:01 | FLKAESKImF | 410.5581 | 3 |
| 10 | HLA-B*08:01 | IMKPKRDGYM | 413.5510 | 3 |
| 11 | HLA-B*08:01 | ImKPKRDGYM | 418.8826 | 3 |
| 12 | HLA-B*08:01 | ImKPKRDGYm | 424.2143 | 3 |
| 13 | HLA-B*08:01 | IMKPKRDGYM | 619.8228 | 2 |
| 14 | HLA-B*08:01 | ImKPKRDGYM | 627.8203 | 2 |
| 15 | HLA-B*08:01 | ImKPKRDGYm | 635.8178 | 2 |
| 16 | HLA-B*08:01 | LHFCamcRSSIM | 384.1881 | 3 |
| 17 | HLA-B*08:01 | LHFCamcRSSIm | 389.5197 | 3 |
| 18 | HLA-B*08:01 | LHFCamcRSSIM | 575.7784 | 2 |
| 19 | HLA-B*08:01 | LHFCamcRSSIm | 583.7759 | 2 |
| 20 | HLA-B*08:01 | LHFCcysRSSIM | 606.7698 | 2 |
| 21 | HLA-B*08:01 | LHFCcysRSSIm | 614.7672 | 2 |
| 22 | HLA-B*08:01 | MFATLQRSSL | 577.3053 | 2 |
| 23 | HLA-B*08:01 | mFATLQRSSL | 585.3028 | 2 |
| 24 | HLA-B*08:01 | YMFLKAESKI | 410.5581 | 3 |
| 25 | HLA-B*08:01 | YmFLKAESKI | 415.8898 | 3 |
| 26 | HLA-B*08:01 | YMFLKAESKI | 615.3336 | 2 |
| 27 | HLA-B*08:01 | YmFLKAESKI | 623.3310 | 2 |

*lowercase m = oxidized methionine, Camc = carbamidomethylated cysteine, Ccys = cysteinylated cysteine

### Database Searching

Mass spectra were interpreted using the Spectrum Mill software package. MS/MS spectra were excluded from searching if they did not have a precursor MH+ in the range of 600-2,000 Da, had a precursor charge >5, or had <4 detected peaks. Merging of similar spectra with the same precursor m/z acquired in the same chromatographic peak was disabled. MS/MS spectra were searched against a database that contained all UCSC Genome Browser genes with hg19 annotation of the genome and its protein coding transcripts (63,691 entries) combined with a full-length GATA3 neoORF sequence and 150 common contaminants. Prior to the database search, all MS/MS had to pass the spectral quality filter with a sequence tag length >2 (i.e., minimum of 3 masses separated by the in-chain mass of an amino acid). A minimum backbone cleavage score was set to 5, and the “ESI QExactive HLA v2” scoring scheme was used. All spectra were searched using a no-enzyme specificity, a fixed modification of cysteine carbamidomethylation (Camc) and the following variable modifications: oxidized methionine (in), pyroglutamic acid, and cysteinylation (Ccys). Precursor and product mass tolerances were set at 0.1 Da and 10 ppm, respectively, and the minimum matched peak intensity was set at 30%. Peptide spectrum matches (PSMs) for individual spectra were automatically designated as confidently assigned using the Spectrum Mill autovalidation module to apply target-decoy based FDR estimation at the PSM rank to set scoring threshold criteria. An auto thresholds strategy using a minimum sequence length of 7, automatic variable range precursor mass filtering, and score and delta Rank1-Rank2 score thresholds were optimized across all nLC-MS/MS runs for an HLA allele yielding a PSM FDR estimate of <1% for each precursor charge state.

### Equation

The experimental monoisotopic molecular weight (MW) of each peptide epitope was calculated according to the following equation where m/z is the mass-to-charge ratio of the peptide epitope detected by the mass spectrometer, z is the charge of the peptide epitope, and 1.007276 is the monoisotopic molecular weight of a proton.

Experimental MW=((m/z)×(z))−((z)×(1.007276)) Equation 8.

### Results

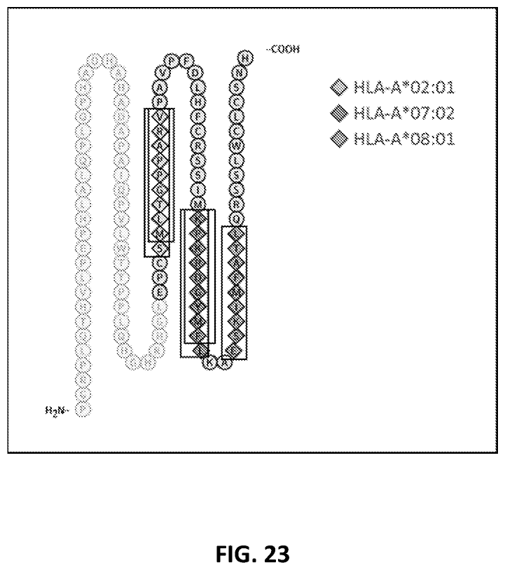

Targeted nLC-MS/MS was used to validate the endogenous processing of peptide epitopes derived from the GATA3 neoORF that were predicted to bind to HLA-A*02:01, HLA-B*07:02, and HLA-B*08:01. Five peptide epitopes derived from the common region of the GATA3 neoORF were detected in HEK293T cells across the three alleles (FIG. 23).

For the HLA-A*02:01 heterodimer, four peptides derived from the common region of the GATA3 neoORF were targeted by nLC-MS/MS. Two peptides from the common region, SMLTGPPARV and MLTGPPARV, were successfully identified by database search and by spectral match to their synthetic peptide counterparts. The theoretical and experimental monoisotopic molecular weights for each peptide epitope with associated mass error are shown in Table 24. Backbone cleavage score and scored peak intensity as reported by the Spectrum Mill database search workflow are listed in Table 25. Backbone cleavage score is indicative of the number of fragment-specific ions generated by HCD whereas scored peak intensity shows the percentage of ion current in the MS/MS spectrum that is explained by the search interpretation.

Table 24 Below Lists Theoretical and Experimental Molecular Weights with Mass Error

| | | | Experi- | |
|---|---|---|---|---|
| | | Theoretical | mental | Mass |
| | | MW | MW | Error |
| Allele | Sequence* | (Da) | (Da) | (Da) |
| HLA-A*02:01 | SMLTGPPARV | 1027.5484 | 1027.5570 | 0.0086 |
| HLA-A*02:01 | MLTGPPARV | 940.5164 | 940.5126 | -0.0038 |
| HLA-B*07:02 | KPKRDGYMF | 1140.5750 | 1140.5748 | -0.0002 |
| HLA-B*07:02 | KPKRDGYMFL | 1253.6590 | 1253.6514 | -0.0076 |
| HLA-B*08:01 | ESKImFATL | 1054.5368 | 1054.5370 | 0.0002 |

*Lowercase m indicates oxidation of methionine.

Table 25 Shows Interpretation Metrics from Database Search

| | Sequence | | |
|---|---|---|---|
| | Coverage Map* | | Scored |
| | Key: / y-ion, | Backbone | Peak |
| | \ b-ion, | Cleavage | Intensity |
| Allele | \| b- & y-ions | Score | (%) |
| HLA-A*02:01 | S M\|L\|T/G/P/P A R V | 5/9 | 78.5 |
| HLA-A*02:01 | M/L\|T/G/P/P A R V | 5/8 | 83.6 |
| HLA-B*07:02 | K/P\|K/R D G Y\|M\|F | 5/8 | 81.1 |
| HLA-B*07:02 | K/P/K R D G Y\M\|F\L | 5/9 | 72.2 |
| HLA-B*08:01 | E S K\I m\F\A/T/L | 5/8 | 73.9 |

*Lowercase m indicates oxidation of methionine.

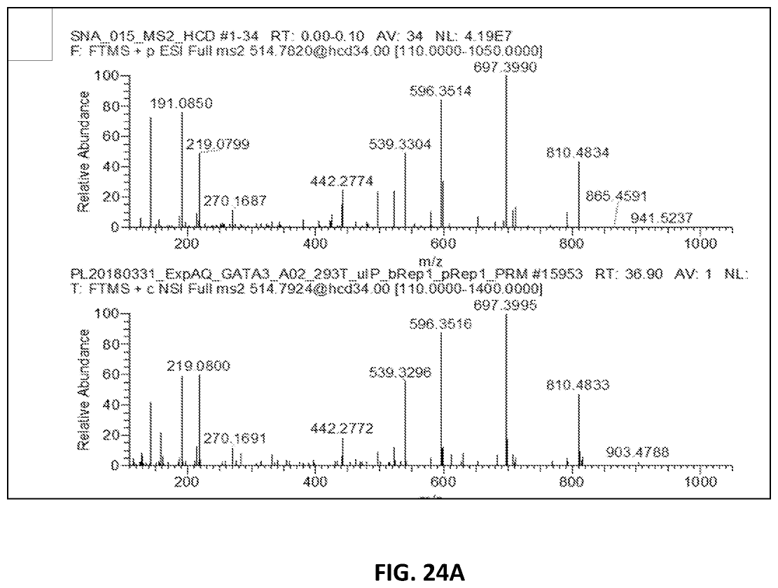

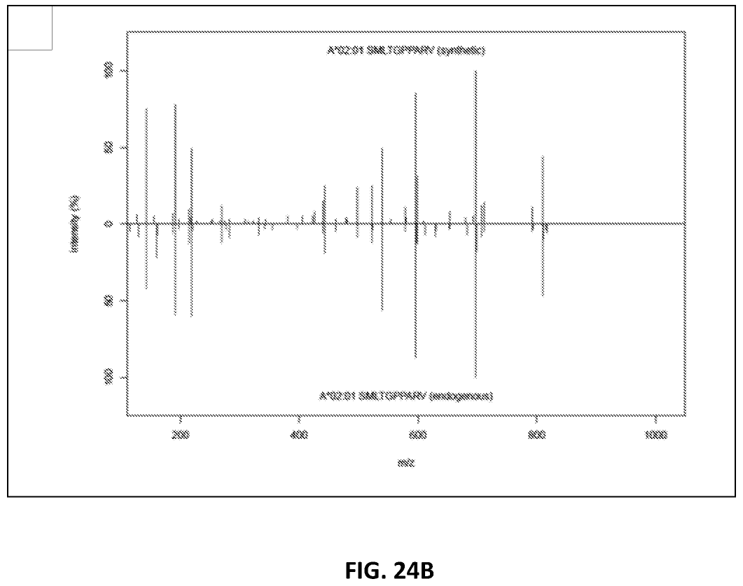

Each MS/MS spectrum acquired on the endogenously processed peptide epitope was matched to an MS/MS spectrum generated using the corresponding synthetic peptide. FIG. 24 shows the spectral comparison of the MS/MS spectrum for endogenously processed peptide epitope SMLTGPPARV (FIG. 24A bottom) and the MS/MS spectrum of its corresponding synthetic peptide (FIG. 24A top). FIG. 24B shows an alternative representation of the identical spectral match. These head-to-toe plots were generated using the top or 50 most abundant ions for 9mer and 10mer peptide epitopes, respectively (http://orgmassspec.github.io/).

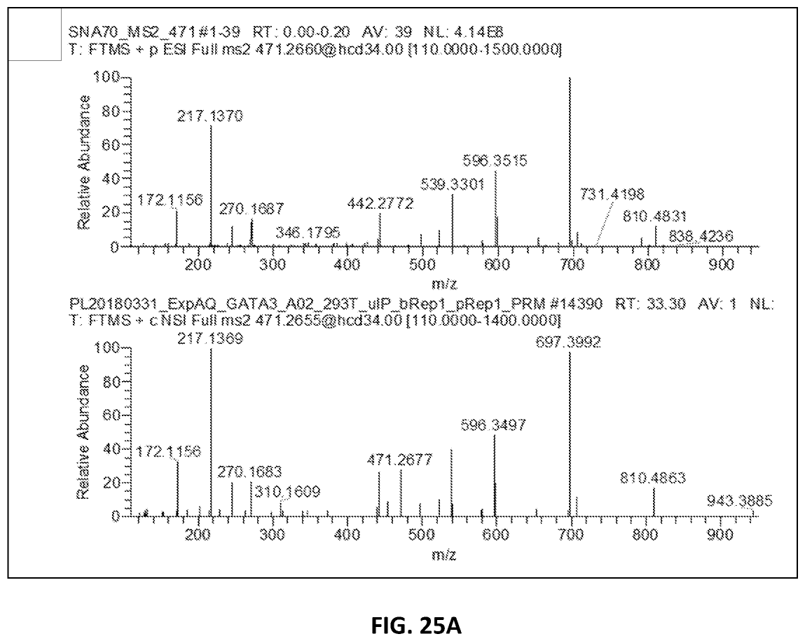

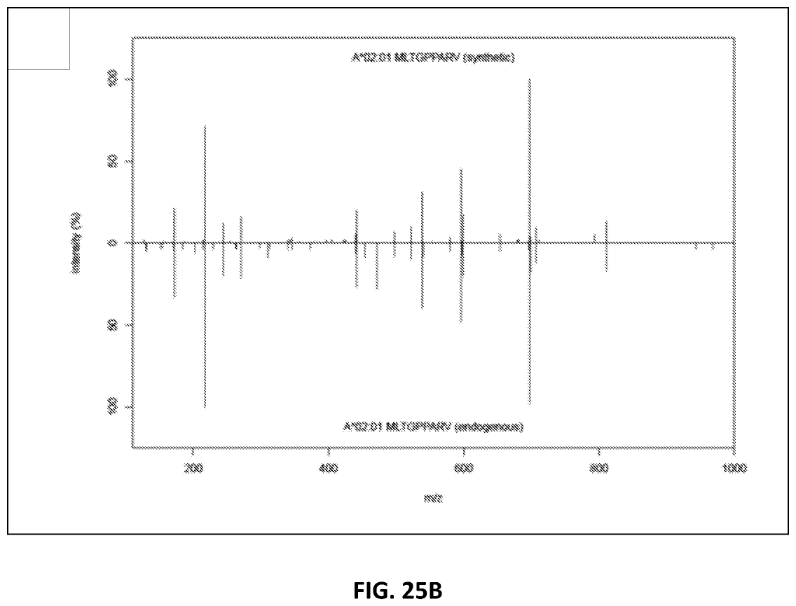

FIG. 25 shows the MS/MS spectral comparison for HLA-A*02:01 endogenously processed peptide MLTGPPARV.

For HLA-B*07:02, five peptide epitopes derived from the common region of the GATA3 neoORF were targeted by nLC-MS/MS. Two peptide epitopes from the common region, KPKRDGYMF and KPKRDGYMFL, were successfully identified by database search and by spectral match to their corresponding synthetic peptides. The theoretical and experimental molecular weights for each peptide epitope with associated mass error are shown in Table 24. Backbone cleavage score and scored peak intensity as reported by the search engine are listed in Table 25.

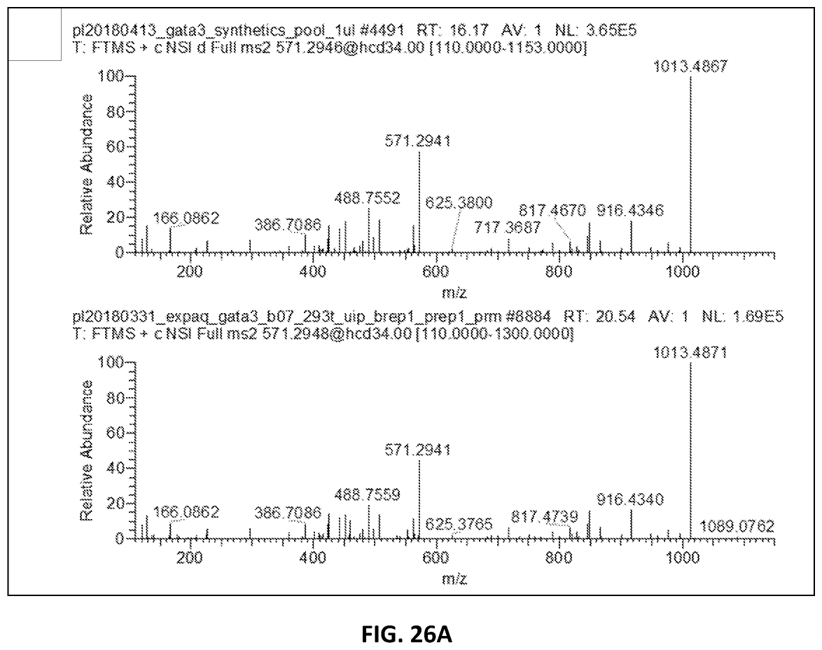

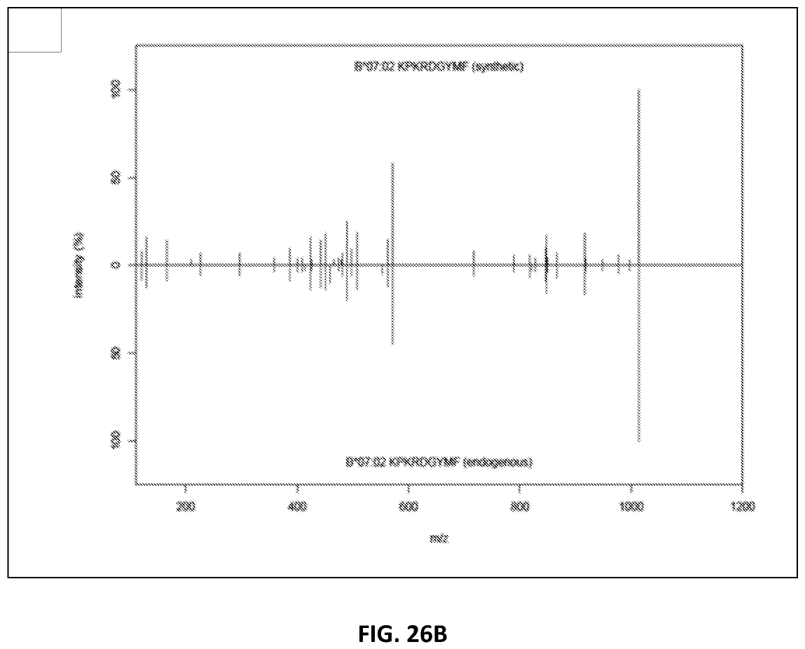

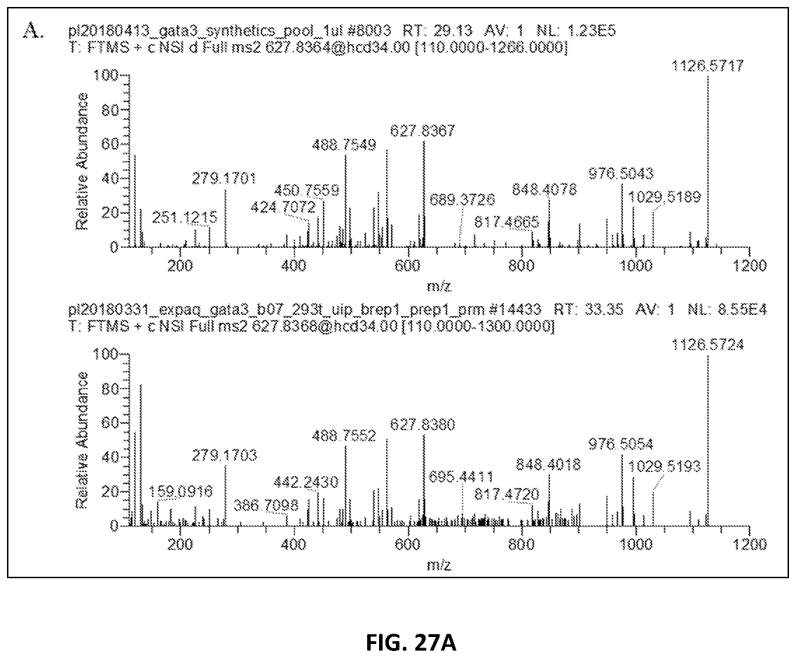

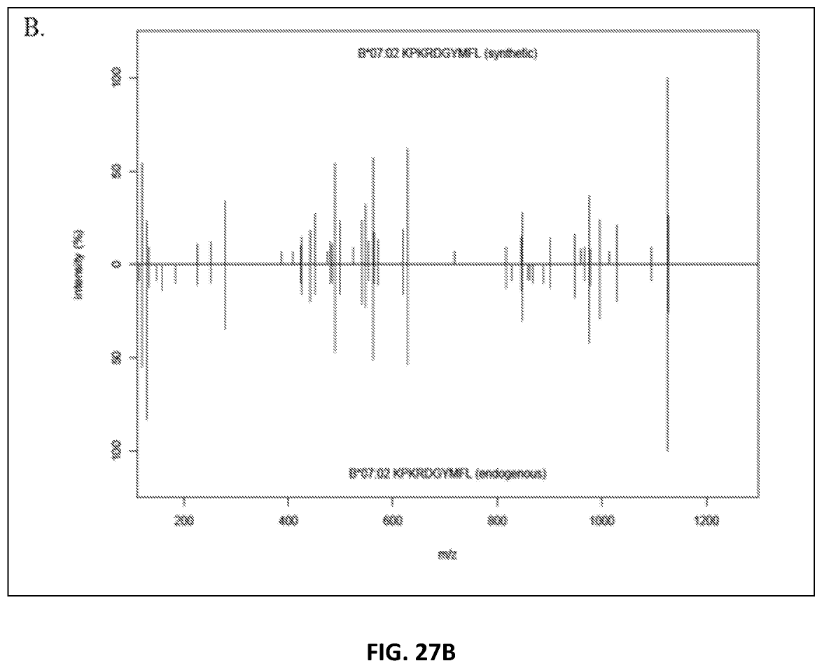

FIG. 26 and FIG. 27 show the spectral comparison for HLA-B*07:02 peptide epitopes KPKRDGYMF and KPKRDGYMFL, respectively.

For HLA-B*08:01, eight peptide epitopes derived from the common region of the GATA3 neoORF were targeted by nLC-MS/MS. One peptide epitope from the common region, ESKIMFATL, was successfully identified by database search and by spectral match to the corresponding synthetic peptide. This peptide was detected with the sulfoxide form of methionine that resulted in a mass shift of 15.999 Da indicative of the addition of oxygen to the side chain. Oxidation of methionine (indicated as lowercase m) to its sulfoxide form is a common result of sample processing. The theoretical and experimental molecular weight for ESKImFATL with associated mass error is shown in Table 24. Backbone cleavage score and scored peak intensity as reported by the search engine are listed in Table 25.

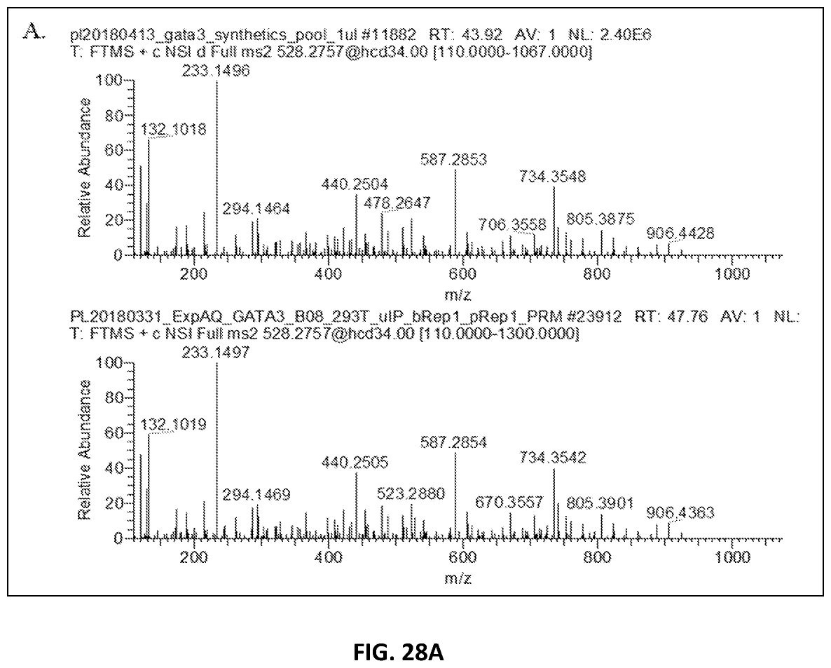

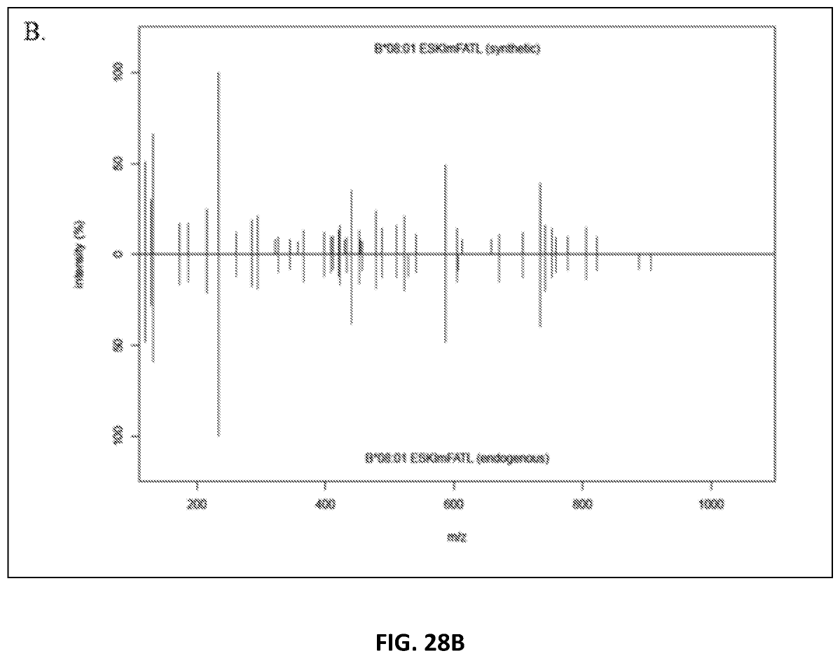

FIG. 28 shows the spectral comparison for HLA-B*08:01 peptide epitope ESKImFATL.

Targeted nLC-MS/MS was used to validate the processing and presentation of five peptide epitopes derived from the common region of the GATA3 neoORF that were predicted to bind to HLA-A*02:01, HLA-B*07:02, and HLA-B*08:01 heterodimers. Class I HLA heterodimers were purified from HEK293T cells stably expressing the GATA3 neoORF using an affinity-tag that was genetically expressed on the alpha chain of each class I HLA heterodimer. Each affinity-tagged heterodimer (BAP-tagged HLA allele) was transiently transfected into GATA3 neoORF expressing HEK293T cells as described in RP19-005. The correct linear sequence for each targeted peptide epitope was confirmed by nLC-MS/MS peptide sequencing. All MS/MS spectra were interpreted with the Spectrum Mill database search workflow that matched experimental MS/MS spectra against peptides from a database comprised of >63,000 entries including the full length GATA3 neoORF. The molecular weight for each observed peptide epitope was calculated within +/−0.01 Da of its theoretical molecular weight. Interpretation of the experimental MS/MS spectra showed >72% of the ion current in the MS/MS spectra could be explained by the sequence-specific fragment ions. Additional confirmation of the endogenously processed peptide epitope sequence was performed by spectral matching to the MS/MS spectrum of the corresponding synthetic peptide that showed identical fragment ion masses and backbone cleavage patterns. Together, targeted nLC-MS/MS confirmed that the HLA-A*02:01 peptide epitopes SMLTGPPARV and MLTGPPARV, the HLA-1*07:02 peptide epitopes KPKRDGYMF and KPKRDGYMFL, and the HLA-1*08:01 peptide epitope ESKIMFATL were endogenously processed in HEK293T cells and subsequently bound by HLA heterodimers.

### Example 23 Immunogenicity on MHC I

This Example evaluates the immunogenicity of the GATA3 neoORF on various, high prevalent HLA alleles. The GATA3 neoORF is a frame shift mutation occurring before the natural stop codon resulting in an extension of the protein by at least 61 novel amino acids. Immunogenicity was evaluated by an in vitro induction of healthy donor (HD) PBMCs against predicted minimal epitopes specific for HLA-A02:01, A03:01, A24:02, B07:02, or B08:01.

### Materials and Methods

| Peptide | | Peptide | Final | Experimental | HLA |
|---|---|---|---|---|---|
| Pool | Sequence | Length | Purity | MW | Allele |
| A02.01 | SMLTGPPARV | 10 | 100% | 1028.1 | A02.01 |
| | YMFLKAESKI | 10 | 100% | 1229.4 | B08.01/A02.01/ |
| | | | | | A24.02 |
| | VLPEPHLAL | 9 | 100% | 988.4 | A02.01 |
| | TLQRSSLWCL | 10 | 98% | 1206.5 | A02.01 |
| | MLTGPPARV | 9 | 100% | 941.3 | A02.01 |
| A03.01 | YMFLKAESK | 9 | 80% | 1116.2 | A03.01 |
| | KIMFATLQR | 9 | 71% | 1107.3 | A03.01 |
| | VLWTTPPLQH | 10 | 85% | 1191.3 | A03.01 |
| A24.02 | YMFLKAESKI | 10 | 85% | 1229.4 | B08.01/A02.01/ |
| | | | | | A24.02 |
| | MFLKAESKI | 9 | 86% | 1066.3 | A24.02 |
| | YMFLKAESK | 9 | 80% | 1116.2 | A03.01/A24.02 |
| | KIMFATLQR | 9 | 71% | 1107.3 | A03.01/A24.02 |
| | VLWTTPPLQH | 10 | 85% | 1191.3 | A03.01/A24.02 |
| B07.02 | KPKRDGYMFL | 10 | 71% | 1254.3 | B07.02 |
| | FATLQRSSL | 9 | 83% | 1022.2 | B08.01/B07.02 |
| | EPHLALQPL | 9 | 81% | 1017.2 | B08.01/B07.02 |
| | KPKRDGYMF | 9 | 73% | 1141.2 | B07.02 |
| | GPPARVPAV | 9 | 81% | 863.0 | B07.02 |
| B08.01-1 | FATLQRSSL | 9 | 83% | 1022.2 | B08.01/A24.02 |
| | ESKIMFATL | 9 | 76% | 1039.2 | B08.01/B07.02 |
| | FLKAESKIM | 9 | 78% | 1066.2 | B08.01/B07.02 |
| | EPHLALQPL | 9 | 81% | 1017.2 | B08.01 |
| B08.01-2 | YMFLKAESKI | 10 | 85% | 1229.4 | B08.01/A02.01/ |
| | | | | | A24.02 |
| | MFATLQRSSL | 10 | 80% | 1153.2 | B08.01/B07.02 |
| | IMKPKRDGYM | 10 | 74% | 1238.4 | B08.01 |
| | FLKAESKIMF | 10 | 71% | 1213.3 | B08.01 |

Table 27 lists healthy donor information

| Healthy | Class I Type | | | | | |
|---|---|---|---|---|---|---|
| Donor ID | HLA-A | | HLA-B | | HLA-C | |
| HD44 | 11:01:01 | 24:02:01 | 13:01:01 | 40:01:02 | 03:04:01 | N/A |
| HD45 | 03:01 | 68:01:02 | 39:01:01 | 51:08:01 | 07:02:01 | 16:02:01 |
| HD48 | 02:06:01 | 29:02:01 | 07:02:01 | 48:01:01 | 07:02:01 | 08:03:01 |
| HD50 | 02:01:01 | 68:03:01 | 35:01:01 | 51:01:01 | 07:02:01 | 16:02:01 |
| HD56 | 01:01:01 | 68:01:02 | 08:01:01 | 40:08 | 03:04:01 | 07:01:01 |

N/A = The donor is homozygous for the particular allele. The epitope targeted alleles are in bold

### Induction Using FMS-Like Tyrosine Kinase 3 Ligand (FLT3L) to Stimulate DCs

FLT3L Stimulation was performed by the following method. PBMCs were thawed and resuspended at 5×10\hat{ }7 cells/mL in AIM-V Media. Benzonase (Sigma-Aldrich 70746) was added at 25-29U/μL and incubated at 37 C for 30 min-1 hr. A CD14/CD25 depletion was performed to remove Monocytes (CD14) and T regulatory cells (CD25) according to the manufacturer's protocol (Miltenyi Biotec, Inc 130-050-201, 130-092-983). Cells at 2×10\hat{ }6 cells/mL were resuspended in AIM-V media with FLT3L (CellGenix 1415-050) at 50 ng/mL and plated 2 mL per well in a 24-well plate overnight. Peptide diluted in AIM-V was added at a final concentration of 2 uM and the wells were mixed gently. The cells were incubated with the peptide for 1 hour at 37° C.

Maturation cocktail with Tumor Necrosis Factor a (TNF-a) (1000U/mL) (CellGenix1406-050), IL-1b (10 ng/mL) (CellGenix1411-050), Prostaglandin-E 1 (PGE-1) (0.5 μg/mL), and IL-7 (0.5 ng/mL) was added and incubated at 37 C overnight. After the overnight maturation cocktail incubation, FBS was added to each well at a final concentration of 10% by volume and mixed. The co-culture was fed every 2-3 days starting at day 5 by carefully replacing 75% of the media with fresh Roswell Park Memorial Institute 1640 (RPMI)+10% FBS with enough IL-7 and IL-15 for a final concentration of 5 ng/mL in the well. For feeds after the first one, the media was replaced with fresh 20/80+10% FBS with enough IL-7 and IL-15 for a final concentration of 5 ng/mL in the well. Begin mDC Generation was begin on day 4.

Mature Dendritic Cell (mDC) Generation

PBMCs were thawed and resuspended at 5×10\hat{ }7 cells/mL in Dendritic Cell (DC) Media (Cellgenix 20801-0500). Benzonase was added (sigma-aldrich 70746) at 25-29U/uL and incubated at 37° C. for 30 min-1 hr.

A Pan Monocyte Isolation was performed according to the manufacturer's protocol (Miltenyi biotec, Inc 130-096-537). Cells were plated in 6-well plates at 3×10\hat{ }6 cells/well in 2 mL DC media with Granulocyte-macrophage colony-stimulating factor (GM-CSF) (800 U/mL) (CellGenix 1412-050) and IL-4 (400 U/mL) (CellGenix 1403-050) and incubated at 37° C. for 5 days.

Peptide loading and maturation was performed in the following manner. The immDCs from the wells were collected by pipetting and pellet by centrifuging at 1200 RPM for 5 minutes. The cells were resuspended at 1 mL in DC media. The cells were separated into pools with 0.2×10\hat{ }6 (or 0.5×10\hat{ }6) cells per well for each pool and incubate for 1 hr at 37 C with 1.6 uM of peptide (0.4 uM final concentration). 800 uL (or 2 mL) of DC media was added with per well to the peptide loaded immDCs and plated in a 6 well plate with the following cytokines and incubated at 37° C. for 2 days: IL-4 (400 U/mL) (CellGenix 1403-050), GM-CSF (800 U/mL) (CellGenix 1412-050), TNF-α (10 ng/mL) (CellGenix1406-050), IL-1b (10 ng/mL) (CellGenix1411-050), PGE-1 (0.5 μg/mL) (Cayman from Czech republic), IL-6 (10 ng/mL) (CellGenix 1004-50).

### Mature Dendritic Cell Mediated Long Term Stimulation (mDC LTS)

On day 12 FLT3L stimulated T Cells were added. The DCs were resuspended in 1 mL 20/80. The coculture wells were harvested and counted and cells were resuspended at 5×10\hat{ }6/mL in 20/80. 1 mL of naïve T cells were added to the mDCs at a ratio of 10:1 (T cells: mDC) with cytokines IL-7 (5 ng/mL) IL-15 (5 ng/mL). Media to a final volume of 5 mL per well was added. The coculture was incubated at 37° C. The co-culture was fed every 2-3 days starting at day 15. Cells were expanded into larger volume flasks as need.

Media to add(mL)=(current vol(mL)×(180−Glucose))/60 Equation 9.

The cultures were restimulated on day 23 on new mDCs. The DCs were resuspended in 1 mL 20/80. The coculture wells were harvested and cells resuspended at 2e6/mL (or 5e6/mL) in 20/80. 1 mL of naïve T cells were added to the mDCs at a ratio of 10:1 (T cells: mDC) with cytokines IL-7 (5 ng/mL) IL-15 (5 ng/mL). Media was added to a final volume of 5 mL per well. The coculture was incubated at 37° C. The co-culture was fed every 2-3 days. Cells were expanded into larger volume flasks as needed.

Media to add(mL)=(current vol(mL)×(180−Glucose))/60 Equation 9:

On day 31 the cells were frozen in 1 mL freeze media (90% FBS, 10% DMSO). Cells were kept overnight at −80° C. in a CoolCell Freeze Container (VWR 75779-816) before transferring them to the liquid nitrogen for long term storage.

### Multimer Generation and Analysis

### Peptide Exchange Monomers

HLA MHC Class I monomers loaded with a UV cleavable peptide were generated internally. The monomers were resuspended at 100 ug/mL in filtered PBS and the assay peptides resuspended at 10 mg/mL in DMSO and the UV cleavable peptide were exchanged with individual assay peptides at a ratio of 1 uL peptide: 50 uL monomer under a UV light for 1 hr at 4° C. Exchanged monomers were spun down at 3600 RPM and the supernatant collected.

### Fluorochrome Conjugation

50 uL pMHC was combined with the streptavidin-labeled fluorochrome on ice for 30 min in the dark according to each of the following fluorochrome's conjugation ratio (CR): PE (BioLegend 405203) (CR: 2); APC (BioLegend 405207) (CR: 3); BV421 (BioLegend 405226) (CR: 2); QD605 (Life Technologies Q10101 MP) (CR: 2); QD705 (Life Technologies Q10161 MP) (CR: 2); BUV395 (BD 564176) (CR: 2); BV650 (BD 563855) (CR: 2). Each pMHC was spilt and conjugated to two individual fluorochromes. Biotin was added (Avidity BIO200)+0.5% azide at a 1:20 ratio and multimers stored at 4° C. in the dark for between 1 and 3 days. Flow cytometry readout obtained on days 11, 22, and post freeze. ˜2×10\hat{ }6 cells were collected in a polypropylene V-bottom 96 well plate in media with Benzonase (sigma-aldrich 70746) at 25-29U/uL and incubate at 37 C for 30 min-1 hr.

Cells were stained with all the following fluorochrome conjugated multimers loaded with the induced peptides in 50 uL filtered Phosphate Buffered Saline (PBS) for 15 min at 37° C. in the dark. PE (BioLegend 405203) 1 uL; APC (BioLegend 405207) 3 uL; BV421 (BioLegend 405226) 1 uL; QD605 (Life Technologies Q10101 MP) 4.5 uL; QD705 (Life Technologies Q10161 MP) 4.5 uL; BUV395 (BD 564176) 3.5 uL; BV650 (BD 563855) 2.5 uL. The samples were stained with the following surface antibodies for 30 min on ice in the dark. Analyzed on the LSR-Fortessa for Cluster of differentiation (CD)8(+)/CD4(−)/CD14(−)/CD16(−)/CD19(−)/Dead(−)/Multimer_1(+)/Multimer_2 (+)/Irrelevant Multimer(−): CD8-FITC (Biolegend 344704) 2 uL, CD4-AF700 (BD 557922) 1 uL, CD14-AF700 (BD 557923) 1 uL, CD19-AFF700 (BD 557921) 1 uL, CD16-AF700 (BD 557920) 1 uL, Live dead stain (Molecular probes L-23101) 0.1 uL.

### Results

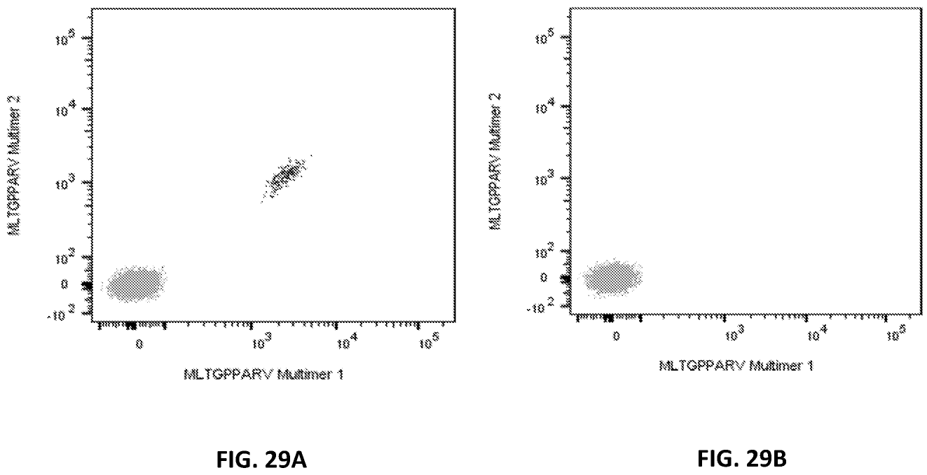

Each sample was stained with two multimers for every peptide the donor was induced against in a unique fluorochrome combination. Positive inductions were determined by at least 10 events positive for at least 1 specific fluorochrome combination and negative for irrelevant fluorochrome combinations. Data was collected after each stimulation and a positive result across two stimulations was considered a positive induction. FIG. 29A and FIG. 29B show representative induction of GATA3 Neoantigen CD8+ Responses.

### Table 28 Shows Percentage of Positive GATA3 Neoantigen CD8+ Responses

| | | Number | | |
|---|---|---|---|---|
| | | of wells | Number | |
| | | with a | of | Percent |
| Inducing | | reactive | wells | positive |
| Peptide | Allele | TCR | tested | inductions |
| MLTGPPARV | A02:01 | 5 | 5 | 100% |
| SMLTGPPARV | A02:01 | 1 | 5 | 20% |
| GPPARVPAV | B07:02 | 1 | 5 | 20% |
| KPKRDGYMF | B07:02 | 1 | 5 | 20% |
| ESKIMFATL | B08:01 | 1 | 5 | 20% |

Table 28 reports the percentage of replicates that resulted in a naïve CD8+ T cell induction confirmed over two independent stimulations.

These results show that there is at least one minimal epitope within the GATA3 neoORF that can generate a CD8+ specific induction in vitro to 4 out of the 5 assayed HLAs. The results indicate broad immunogenicity of the GATA3 neoORF across all HLAs and identify immunogenic minimal neoantigen epitopes specific to the high prevalent HLAs.

### Example 24 Immunogenicity on MHC II

This Example evaluates the CD4 immunogenicity of longmer peptides (>15 amino acids) specific to the GATA3neoORF. To predict in vivo CD4 immunogenicity an in vitro induction assay was used against five different healthy donors with minimal HLA-Class II overlap.

### Materials and Methods

### Table 29 Below Lists Inducing Peptides

| Peptide | | Peptide | Final | Molecular |
|---|---|---|---|---|
| Pool | Sequence | Length | Purity | Weight |
| L1 | PGRPLQTHVLPEPHLALQPLQPHADHA | 27 | 95% | 2971.2 |
| | APAIQPVLWTTPPLQHGHRHGLEPCS | 26 | 90% | 2843.4 |
| | PPARVPAVPFDLHFCRSSIMKPKRD | 25 | 93% | 2865.8 |
| L2 | EPHLALQPLQPHADHAHADAPAIQPV | 26 | 92% | 2774.2 |
| | TPPLQHGHRHGLEPCSMLTGPPARVPA | 27 | 96% | 2857.8 |
| | DLHFCRSSIMKPKRDGYMFLKAESK | 25 | 88% | 2989.0 |
| | KAESKIMFATLQRSSLWCLCSNH | 24 | 100% | 1810.0 |
| L3 | HADHAHADAPAIQPVLWTTPPLQH | 24 | 94% | 2624.1 |
| | EPCSMLTGPPARVPAVPFDLHFCR | 24 | 98% | 2641.4 |
| | KPKRDGYMFLKAESKIMFATLQRS | 24 | 98% | 2846.8 |

### Table 30 Below Provided Healthy Donor Information

| | | Healthy Donor ID | | | | |
|---|---|---|---|---|---|---|
| | | HD41 | HD44 | HD63 | HD47 | HD56 |
| Class II Allele | HLA-DPA1 | 01:03:01 | 02:02:02 | 01:03:01 | 01:03:01 | 01:03:01 |
| | | 01:04:01 | 02:02:02 | 01:03:01 | 01:03:01 | 02:01:02 |
| | HLA-DPB1 | 05:01:01 | 05:01:01 | 04:01:01 | 04:01:01 | 01:01:01 |
| | | 107:01 | 05:01:01 | 04:01:01 | 15:01:01 | 04:02:01 |
| | HLA-DQA1 | 03:03:01 | 01:02:02 | 02:01:01 | 05:01:01 | 03:01:01 |
| | | 01:03:01 | 05:08 | 04:01:01 | 05:05:01 | 05:01:01 |
| | HLA-DQB1 | 01:03:01 | 03:01:01 | 02:02:01 | 02:01:01 | 02:01:01 |
| | | 01:03:01 | 05:02:01 | 04:02:01 | 03:01:01 | 03:02:01 |
| | HLA-DRB1 | 04:05:01 | 12:01:01 | 07:01:01 | 03:01:01 | 03:01:01 |
| | | 11:01:01 | 16:02:01 | 08:02:01 | 11:02:01 | 04:04:01 |
| | HLA-DRB345 | 02:02:01 | 01:01:02 | 01:01:01 | 02:02:01 | 01:01:02 |
| | | 01:03:01 | 01:01:01 | Not Present | 02:02:01 | 01:03:01 |

Mature Dendritic Cell (mDC) Generation

### Monocyte Isolation

PBMCs were thawed and resuspended at 5×10\hat{ }7 cells/mL in Dendritic Cell (DC) Media (Cellgenix 20801-0500). Benzonase was added (sigma-aldrich 70746) at 25-29U/uL and incubated at 37° C. for 30 min-1 hr. A Pan Monocyte Isolation was performed according to the manufacturer's protocol (Miltenyi biotec, Inc 130-096-537). The cells were plated in 6-well plates at 3×10\hat{ }6 cells/well in 2 mL DC media with GM-CSF (800 U/mL) and IL-4 (400 U/mL) and incubate at 37° C. for 5 days.

### Peptide Loading and Maturation

The immDCs were collected from the wells by pipetting and pelleted by centrifuging at 1200 RPM for 5 minutes. The cells were resuspended at 1 mL in DC media. The cells were separated into pools with 0.2×10\hat{ }6 (or 0.5×10\hat{ }6) cells per well for each pool and incubate for 1 hr at 37° C. with 1.6 uM of peptide (0.4 uM final concentration). 800 uL (or 2 mL) of DC media was added with per well to the peptide loaded immDCs and plated in a 24 (or 6) well plate with the following cytokines and incubated at 37° C. for 2 days; IL-4 (400 U/mL) (CellGenix 1403-050), GM-CSF (800 U/mL) (CellGenix 1412-050), TNF-α (10 ng/mL) (CellGenix1406-050), IL-1b (10 ng/mL) (CellGenix 1411-050), PGE-1 (0.5 μg/mL) (Cayman from Czech republic), IL-6 (10 ng/mL) (CellGenix 1004-50)

### Long Term Stimulation (LTS)

Naïve T Cells were added. PBMCs were thawed and resuspended at 5×10\hat{ }7 cells/mL in DC Media (Cellgenix 20801-0500). Benzonase was added (sigma-aldrich 70746) at 25-29U/uL and incubate at 37° C. for 30 min-1 hr.

A CD14/CD25 depletion was performed to remove Monocytes (CD14) and T regulatory cells (CD25) according to the manufacturer's protocol (Miltenyi Biotec, Inc 130-050-201, 130-092-983). 1 mL of naïve T cells were added to the mDCs at a ratio of 10:1 (T cells: mDC) with cytokines IL-7 (5 ng/mL; CellGenix) IL-15 (5 ng/mL; CellGenix). The coculture was incubated at 37° C. The mDCs were either resuspended in 20/80 or kept in the DC media with cytokines.

The co-culture was fed every 2-3 days starting at day 5 following either method 1 or method 2 below (2.3.2.1.1 or 2.3.2.1.2). Cells were expanded into larger volume flasks as need. For method 1,

media to add was calculated as (mL)=(current vol(mL)×(180−Glucose))/60. For method 2, glucose meter was used to check if the media is yellow. If glucose remains high (>90 mg/dL), 100 uL of 20×IL-7 and IL-15 was added to the well. If glucose is low (<90 mg/dL), the cells were expanded to 6 well plate (4 mL/well) and supplemented with 1×IL-15 and IL-7. If glucose is very low (<60 mg/dL), expanded to 6 mL/well in a 6-well plate. On days 6 and 13 or 14 new mDCs were generated

On days 13 and 21 or 22 the cultures on new mDCs were restimulated. The coculture wells were harvested and counted and resuspended cells at 2×10\hat{ }6/mL (or 5×10\hat{ }6/mL) in 20/80. 1 mL of naïve T cells were added to the mDCs at a ratio of 10:1 (T cells: mDC) with cytokines IL-7 (5 ng/mL) IL-15 (5 ng/mL). The coculture was incubated at 37° C. The mDCs were either resuspended in 20/80 or kept in the DC media with cytokines. On day 28 or 29 the cells were frozen in 1 mL freeze media (90% FBS, 10% DMSO). The cells were kept overnight at −80 C in a CoolCell Freeze Container (VWR 75779-816) before transferring them to the liquid nitrogen for long term storage.

### CD4 Recall Assay

Either new mDCs were generated or fresh PBMCs were thawed from the same induction donor. Plate the cells at 0.26/well of PBMCs and 0.02×10\hat{ }6/well of mDCs in a 96-well u-bottom plate with peptide or DMSO at 0.8 uM final concentration. Induction sample was added at a 1:1 induction: PBMC or 10:1 induction: mDC ratio to the wells and incubate at 32 C for 20-24 hours

### Flow Cytometry Readout

Golgi stop (BD 554724) and golgi plug (BD 555029) was added to the cultures according to the manufacture's protocol and incubate for 4 hours. Cells were stained with CD4-BV786 (BD 563877), CD8-AF700 (BD 561453), CD14-FITC (BD 340682), CD16-FITC (BD 340704), CD19-FITC (BD 340864), Live dead stain (Molecular probes L-23101) for 20 minutes. The samples were fixed using Fixation/Permeabilization kit (BD 554714) according to the manufacture's protocol. The cells were stained with IFN-γ-PE (Biolegend 502508) for 20 minutes. Analysis was done on the LSR-Fortessa for CD4 (+)/CD8 (−)/CD14 (−)/CD16 (−)/CD19 (−), Dead (−)/IFNγ (+).

### Results

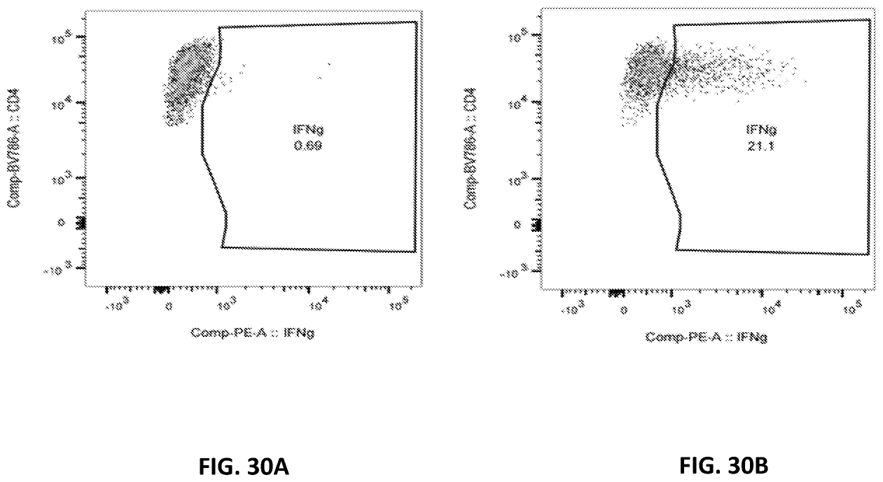

Using flow cytometry, antigen specific, CD4 T cells were identified (FIG. 30A and FIG. 30B). At least one induction to a GATA3 neoORF specific peptide in every healthy donor tested was identified.

Specific reactive peptides were identified by recalling the induced cells against individual peptides and comparing to a recall without peptide. Each induction sample was run initially against the inducing pools and the positive samples were subsequently recalled against individual peptides. All samples were run on duplicate plates and if the average CD4+/IFNy+ percentage of the induction samples with peptide was greater than 2% compared to the same sample without peptide the sample was considered a hit.

### Table 31 Shows Percentage of GATA3 Neoantigen CD4 Responses

| Inducing Peptide | HD41 | HD44 | HD56 | HD47 | HD63 |
|---|---|---|---|---|---|
| EPHLALQPLQPHADHAHADAPAIQPV | 0% | 20% | 0% | 0% | 0% |
| APAIQPVLWTTPPLQHGHRHGLEPCS | 0% | 0% | 0% | 20% | 0% |
| PPARVPAVPFDLHFCRSSIMKPKRD | 0% | 20% | 20% | 0% | 0% |
| DLHFCRSSIMKPKRDGYMFLKAESK | 40% | 0% | 20% | 40% | 20% |
| KPKRDGYMFLKAESKIMFATLQRS | 0% | 40% | 0% | 0% | 0% |
| LKAESKIMFATLQRSSLWCLCSNH | 20% | 0% | 0% | 0% | 0% |

Sequences in bold are in the region of the GATA3 NeoOrf common to all patients

At least one CD4 specific response was observed to the common region of the GATA3 neoORF in all healthy donors tested. These donors had a wide range of MHC Class II HLA alleles, indicating that the ability to generate a CD4 GATA3 response is not allelic dependent.

### Example 25 Functional Assays with Induced CD8+ T Cells

This Example shows the ability of CD8+ T cells specific for neoantigens derived from the GATA Binding Protein 3 (GATA3) novel open reading frame (neoORF) to kill cells harboring the appropriate GATA3 neoORF mutation. The induction of the neoantigen specific CD8+ T cells is described previously in Example 23 and the generation of the cell line harboring the GATA3 neoORF mutation is described in Example 21.

In this Example, CD8+ T cells specific for a single epitope from the GATA3 neoORF presented on HLA-A02:01 (covered by peptide sequence MLTGPPARV) were selected for this detailed analysis. Briefly, the induced CD8+ T cells were co-cultured with target cells either transduced with the GATA3 neoORF or unmanipulated as a negative control. After co-culture, the target cells were evaluated for their expression of Caspase 3, a marker of cell death, as a measure of cytotoxicity by the induced CD8+ T cells. Increased Caspase 3 on the target cells expressing the GATA3 neoORF relative to unmanipulated cells represents specific killing due to the cognate epitope being presented on the surface of the target cells. In addition, the CD8+ T cells were evaluated for the expression of CD107a, a T cell activation marker, to measure antigen specific T cell activation. To further evaluate antigen specific recognition of GATA3 induced PBMC, IFN-γ, a cytokine produced by CD8+ cytotoxic T cell upon antigen recognition, was also measured in the supernatant.

In total, four different CD8+ T cell populations specific for a GATA3 neoORF epitope on HLA-A02:01 were tested for their ability to kill target cells harboring the mutation. In all four cases, increased Caspase 3 was observed on the target cells harboring the GATA3 neoORF relative to target cells without the mutation. The increase in Caspase 3 demonstrates ability of CD8+ T cells specific for epitopes from GATA3 neoORF to kill cells harboring the GATA3 neoORF mutation.

### Materials and Methods

### Cytotoxicity Assay

HEK 293T cell lines were purchased from the American Type Culture Collection (Rockford, Md., USA) and maintained in DMEM, 10% FBS, and Pen/Strep medium. GATA3 gene encoded lentivirus was generated and transduced to HEK 293T cells. The GATA3 transduced HEK 293T cells were maintained under 1 μg/mL of puromycin in complete media for more than 2 weeks. Further details are described in Example 21.

1×10⁷ target cells in 1 mL were added to 1 μL of Tag-it Violet (Biolegend) followed by incubation in 5% CO₂ incubator for 20 minutes, washing 5 mL of culture media with 10% FBS twice, and resuspending cells in at 1×10⁶ cells of culture media.

The induced PBMC vials were thawed by placing in 37° C. water bath. Then 1 mL of FBS was added to each vial. The cells were transferred to 50 mL conical tube containing 15 ml culture media of AIM-V, 10% FBS, and Pen/Strep medium. The cells were centrifuged at 1500 rpm for 5 min and resuspended in 5 mL of culture media. The cells were rested for 1 hour and 30 min in 37° C., 5% CO₂ incubator before adding 5 μL of Benzonase (Millipore Sigma) and further incubating for 30 min in 37° C., 5% CO₂ incubator. The incubated cells were centrifuged again, and after removing supernatant were resuspended in 5 mL of AIM-V media. The cell number was counted using Vi-CELL counter (Beckman coulter).

The cells were centrifuged and resuspended in the 40 μL of MACS buffer per 1×10⁷ target cells. CD8+ positive cells were negatively enriched according to human CD8+ T cell isolation kit (Miltenyi Biotec). The CD8+ cells were resuspended at 2.5×10⁶ cells in 1 mL of AIM-V media. 5×10⁴ of target cells per well were seeded in 50 μL of culture media on 96 well flat bottom plate and cultured for overnight in 37° C., 5% CO₂ incubator. GATA3 induced and CD8+ enriched cells were seeded over the target cells at 2.5×10⁵ cells per well in 100 μL of AIM-V media. The co-culture cells were incubated for 6 hours in 37° C., 5% CO₂ incubator.

The culture supernatants of co-culture were harvested and assessed IFN-γ concentration with V-PLEX Human IFN-γ assays according to manufacturer's protocol (Meso Scale Discovery).

The suspension cells were transferred to a new staining plate and the adherent cells were transferred after adding 50 μL of trypsin per well, incubation at 37° C. and resuspending with AIM V media. The cells were combined, centrifuged and washed with FACS buffer. 50 μL of antibody mixture (anti-CD3-BUV8052, anti-CD4-BV711, anti-CD107a-BV786, anti-CD8+-PE-cy5, and IR dye Live/Dead; BD) were added to each sample following by incubation for 30 min on ice. The cells were washed with 100 μL of FACS buffer. Caspase-3 intra cellular staining was performed according to manufacture manual of Cytofix/Cytoperm kit (BD) with 2 μL of Caspase-3 antibody. The stained cells were analyzed by Fortessa II (BD).

### Results

### Cytotoxicity Assay by GATA3 Induced PBMC

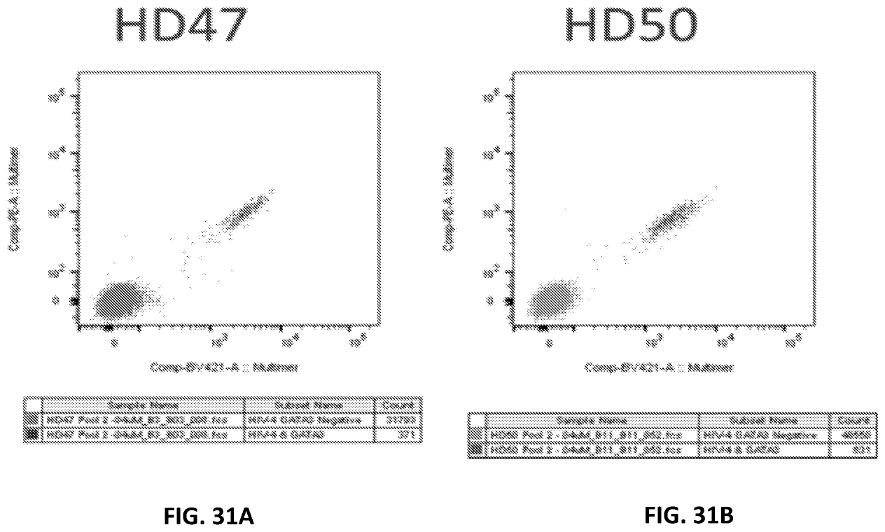

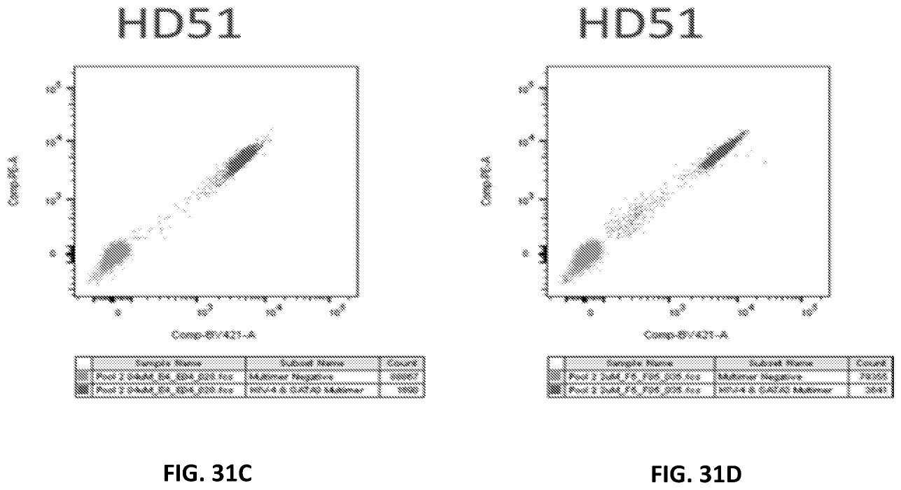

Three different healthy donor PBMCs (HD47, HD50 and HD51) were induced with the GATA3 HLA-A:02 neoantigen peptide MLTGPPARV by long term stimulation method. This epitope:allele combination was determined optimal for testing in cytotoxicity assay as compared to other epitope:alleles related to GATA3 neoORF considering allele frequency and cell counts. FIGS. 31A-31D show GATA3 specific CD8+ T cells by multimer staining. These T cells were selected as effector cells for cytotoxicity assay.

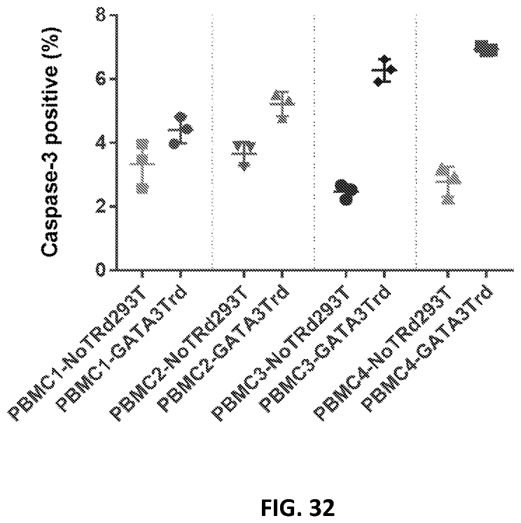

GATA3 transduced HEK293T cells were used as target cell (Example 21). Also, non-transduced HEK 293T cells were used for negative control. After 6 hours co-culture of effector cells and target cells, averages of 3.3%, 3.7%, 2.5% and 2.8% of Caspase-3 positive cells were found for the 4 experiments in non-transduced target cells, and averages of 4.4%, 5.2%, 6.3% and 6.9% of Caspase-3 positive cells were seen in GATA3 transduced cells, respectively (FIG. 32). Significantly higher Caspase-3 positive target cells were observed at co-culture with GATA3 induced PBMCs from HD51 (FIG. 33). The higher frequency of CD107a expressed CD8+ T cells were observed in GATA3 transduced HEK293T cells co-culture condition with 2 of GATA induced healthy donor PBMC (sample 1 and sample 2) (FIG. 34). Higher level of IFN-γ were detected in the same condition of co-culture with 2 of GATA induced healthy donor PBMC (sample 1 and sample 2) (FIG. 35).

An in vitro cytotoxicity assay was utilized to evaluate the ability of CD8+ T cells specific for the GATA3 neoORF to recognize and kill cells harboring the GATA3 frameshift mutation. PBMCs including T cells specific for GATA3 frameshift neoantigens on HLA-A02:01 derived from healthy donors stimulated with the GATA3 frameshift peptide MLTGPPARV were tested as representative of types of T cells that might be induced from a GATA3 frameshift vaccine. These T cells led to tumor cell death as confirmed by assaying the presence of target cell death marker, Caspase-3. Results from the CD8+ T cell activation marker CD107a and cytokine IFN-γ assays further suggest these peptide-induced T cells can recognize and kill cells that naturally process and present GATA3 frameshift neoantigens.

### Example 26 TCR Cloning and Functional Assays

The Example shows cloning of the T cell receptor (TCR) from a CD8+ T cell specific for a neoantigen from the GATA3 neoORF on HLA-A02:01 and functional assays. The induction of neoantigen-specific CD8+ T cells is described in Example 23. The specific CD8+ T cells were isolated using fluorescence-activated cell sorting (FACS) and the TCR sequence was identified using the 10× Genomics and MiSeq platforms. The selected TCR was then recombinantly expressed in a T cell line for both functional characterization of the TCR and evaluation of the processing and presentation of the neoantigen on the surface of a cell harboring the GATA3 neoORF mutation, the generation of which is described in Example 21.

The TCR was characterized to have an avidity below 40 nM. This demonstrates that it is possible to generate CD8+ T cells with TCRs to GATA3 neoantigens. Further, the TCR is able to recognize the processed and presented neoantigen on the surface of HEK293T cells harboring the GATA3 neoORF mutation, which supports the results in Example 22, demonstrating the processing and presentation of GATA3 neoantigens on the surface of cells harboring the mutation.

### Materials and Methods

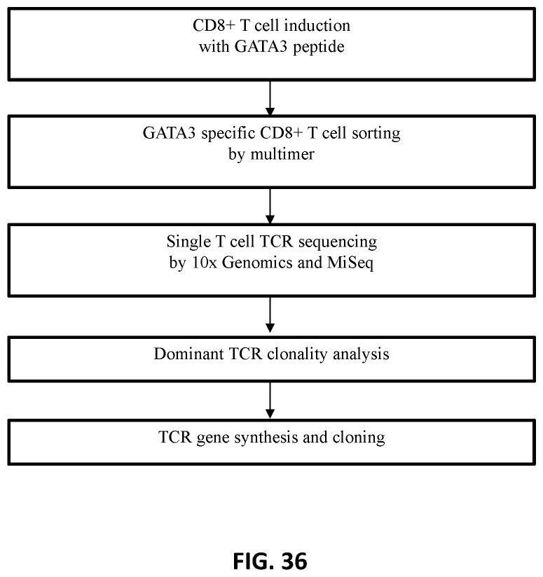

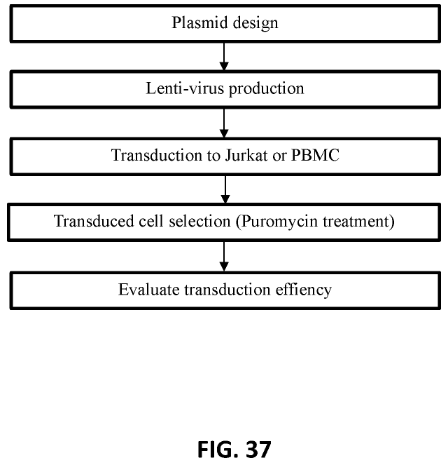

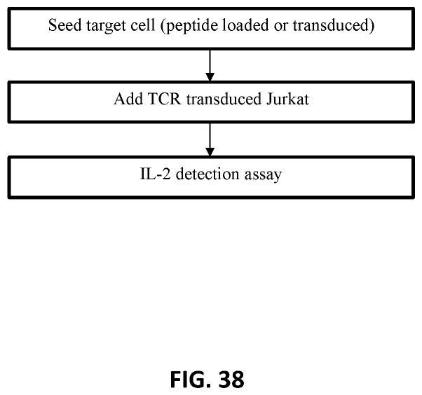

FIGS. 36-38 shows an overview of TCR cloning and functional assay. GATA3 CD8+ T cell sorting by multimer

GATA3 neoORF specific T cells were induced and expanded (Example 23). The induced cells were stained with GATA3 9mer peptide multimer and surface antibodies (CD8, CD4, CD14, CD16, CD19 and a near-IR fluorescent reactive dye). GATA3 specific T cells, which were GATA3 multimer and CD8 positive, were sorted by FACS ARIA fusion (BD) and collected into 1.5 mL tube containing 2% FBS in PBS.

### Single T Cell TCR Sequencing by 10× Genomics and MiSeq

After sorting, the collected cells were immediately processed for single cell barcoding and generating cDNA with Chromium Single Cell V(D)J Reagent Kits (10× Genomics). TCR sequence enrichment and library construction were performed according to the manufacturer's protocol. The sequencing was performed at 10 μM scale with MiSeq 300 cycles reagent kit and MiSeq (Illumina). Analysis of TCR sequence and clonality were made employing Cell ranger software and Loupe VDJ browser (10× Genomics).

### TCR Gene Synthesis and Cloning

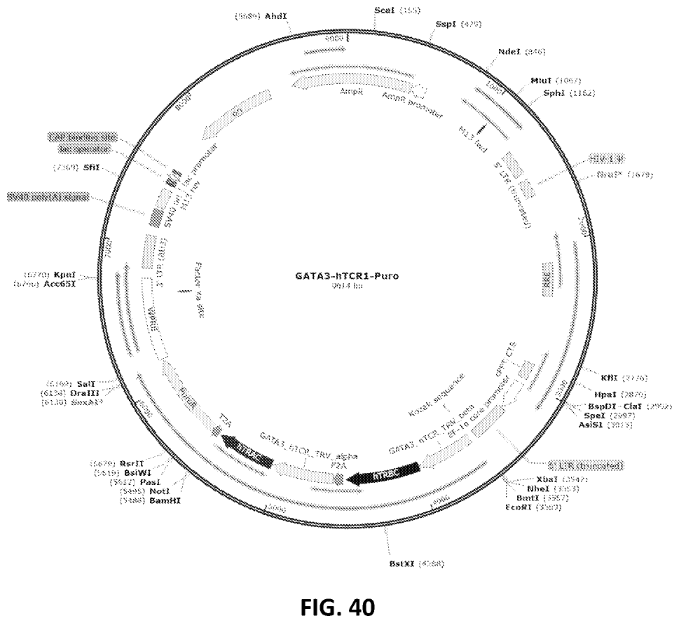

The selected TCR sequences were codon-optimized for mammalian system. The TCR DNA sequences were synthesized and cloned to lenti-virus vector (pCDH-EF1α-Puro, System Biosciences) by GENEWIZ (NJ, USA). The lenti-virus vector contained EF1α promotor followed by TCR beta, furin cleavage site, F2A, TCR alpha, T2A, and puromycin resistance site (FIG. 40)

### Lenti-Virus Production

To generate lenti-virus encoded GATA3 neoORF TCR gene, the lenti-virus vector, package plasmids and the fresh HEK 293T cells (ATCC) were used by transfection method. The transfection and harvest details are described in Example 21.

### Transduction to Jurkat Cells or PBMC

The modified Jurkat (J.RT3-T3.5, ATCC) cells, which lack the TCR beta, were modified to express the CD8+ alpha chain by lenti-virus encoding the CD8+ alpha gene. The modified Jurkat cells were used for transduction of GATA3 TCR lenti-virus. 1.8×10⁶ Jurkat cells were seeded in 1.2 mL of RPMI-1640 media with 10% FBS and 6 μg/mL of polybrene in 24 well plate. After 0.6 mL of GATA3 specific TCR lenti-virus was added to the cells, the plate was centrifuged at 2,400 rpm for 45 minutes at 32° C. The cells were incubated in 5% CO2 incubator for 24 hours. The transduced Jurkat cells were maintained in RPMI-1640 media with 10% FBS with 1 μg/mL of Puromycin for 10 days.

IL-2 Release Assay with Peptide Titration

To evaluate the sensitivity of the TCR that was cloned into the Jurkat cells, a peptide titration assay was performed. Jurkat cells secret IL-2 specifically in response to TCR signaling. Because the Jurkat cells employed in this assay lack an endogenous TCR beta, the TCR on the surface of these cells is specifically the cloned TCR. Peptides were added across a broad range of concentrations to HEK293T target cells with the relevant HLA (HLA-A02:01) but not the GATA3 mutation to evaluate the maximal IL-2 secretion, and to estimate the concentration of peptide required to release 50% of this maximal secretion (EC50). This EC50 is taken as the avidity of the TCR. 20,000 unmodified HEK 293T cells which endogenously express HLA:A02.01 were seeded on 96 well plate with addition of 100 μL GATA3 peptide or irrelevant peptide ranging from 20 μM to 2 μM concentration in DMEM with 10% FBS. After overnight incubation at 37° C., 200,000 GATA3 neoORF specific TCR transduced Jurkat cells were added into each well at a 10:1 ratio of TCR transduced Jurkat cells to peptide loaded HEK 293T cells. The co-culture was incubated in 5% CO2 incubator at 37° C. for 24 hours. 50 μl of supernatant from each well were harvested and the concentration of human IL-2 were measured by Meso scale discovery kit according to the manufacturer's protocol.

IL-2 Release Assay with GATA3 Mutation Transduced Target Cell

To evaluate the ability of the TCR to recognize a truly processed and presented neoantigen, a co-culture of TCR-transduced Jurkat cells and HEK 293T target cells. In this system, though, no peptides were exogenously added. Instead, cell lines transduced with either the GATA3 neoORF or an irrelevant gene were utilized as targets. In this way, for the TCR-transduced Jurkat cells to recognize its targets, the GATA3 neoantigen has to be processed and presented on the surface of the target cell.

20,000 of GATA3 mutation or irrelevant gene transduced HEK 293T cells were seeded on 96 well plate. After overnight incubation, 200,000 of GATA3 specific TCR transduced Jurkat cells were added into each well. The co-culture was incubated in 5% CO2 incubator, 37° C. for 24 hours. 50 μl of supernatant from each well were harvested and the concentration of human IL-2 were measured by Meso scale discovery kit according to the manufacturer's protocol (Meso Scale Discovery).

### Results

### GATA3 Specific TCR Jurkat Cells

### GATA3 Specific CD8⁺ T Cell Sorting

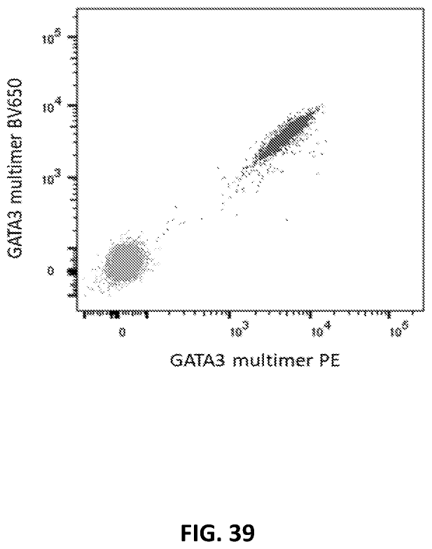

2.1% of GATA3 specific CD8+ T cells were detected in the well number 5 of healthy donor 42 by GATA3 HLA-A02 multimer after long term stimulation with GATA3 neoORF peptide MLTGPPARV. The multimer double positive 5,402 cells were sorted by FACSARIA (FIG. 39). The sorted cells were single cell barcoded with 10× Genomics V(D)J kit. The TCR alpha and beta paired sequences were analyzed with Loupe V(D)J browser. The dominant clonotype (clonotype1) has sequences CALDIYGNNRLAF and CASSLDFVLAGSYSYEQFF of CDR3 TCR alpha and beta amino acid sequences, respectively. Clonotype 2 has the same TCR beta sequence as clonotype1 without the sequence of TCR alpha. Clonotype 4 has same TCR alpha sequence as clonotype 1 without the sequence of TCR beta. The sum proportion of clonotype 1, 2 and 4 was 82.5% of all TCR clonotypes and other clonotypes were less than 1% (Table 32).

### Table 32 Below Shows Exemplary GATA3 Specific TCR Clonotype Analysis

| clonotype_id | frequency | proportion | CDR3 Amino acid sequence |
|---|---|---|---|
| clonotypel | 1178 | 48% | TRA:CALDIYGNNRLAF;TRB:CASSLDFVLAGSYSYNEQFF |
| clonotype2 | 848 | 34% | TRB:CASSLDFVLAGSYSYNEQFF |
| clonotype4 | 12 | 0.5% | TRA:CALDIYGNNRLAF |
| clonotype3 | 12 | 0.5% | TRA:CAEKVPNTGNQFYF;TRB:CASSSLGTVRTEAFF |
| clonotype5 | 10 | 0.4% | TRA:CAVEAYNFNKFYF;TRB:CASRSENTIYF |
| clonotype6 | 10 | 0.4% | TRA:CILSDSGNTPLVF;TRB:CASSDWAVSGNTIYF |
| clonotype7 | 9 | 0% | TRA:CAGAANAGGTSYGKLTF;TRB:CASSQAQGANYGYTF |
| clonotype9 | 7 | 0% | TRA:CAEIPTFSGGYNKLIF;TRB:CASSLAGQETQYF |
| clonotype8 | 7 | 0% | TRA:CLRGGSTLGRLYF;TRB:CASSLYPTGGSGMDEQYF |
| clonotype10 | 6 | 0% | TRA:CAVRDGNTGGFKTIF;TRB:CASSELKTGGAFF |

### GATA3 Specific TCR DNA Synthesis and Cloning

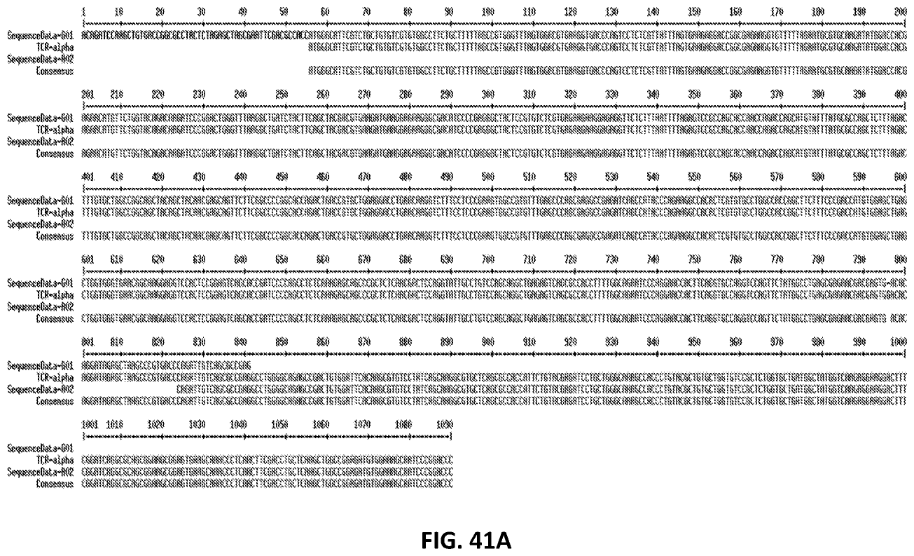

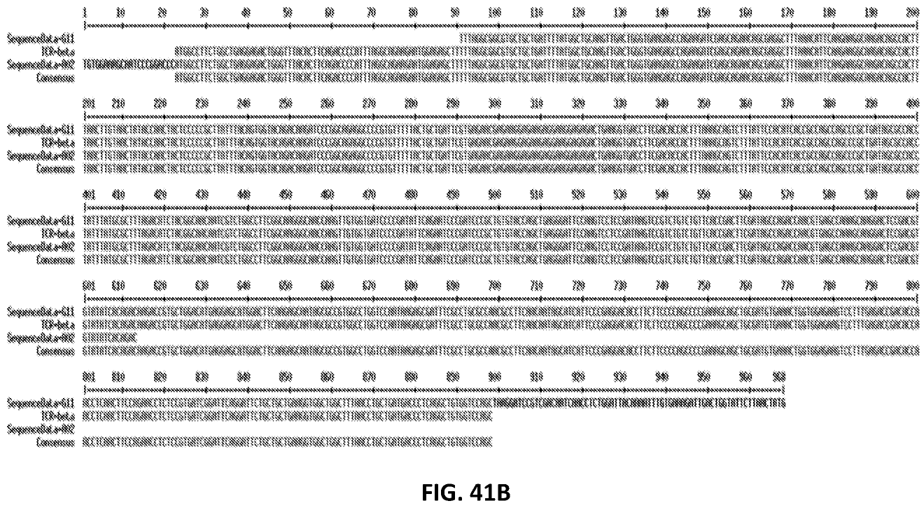

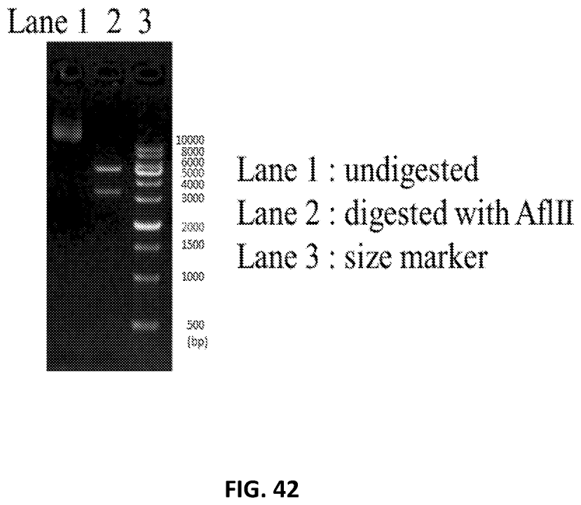

Clonotype 1 TCR alpha and beta sequence were codon optimized according to human codon usage frequency for maximum expression in human cell line and PBMC (Table 32). The TCR gene encoded lenti-virus plasmid (FIG. 40) was evaluated by DNA sequencing and restriction enzyme digest. DNA sequence data of final GATA3 neoORF specific TCR encoded plasmid is 100% matched with TCR alpha and beta codon optimized sequence (bold font in FIG. 41). After the restriction enzyme AfIII digestion, two DNA bands were observed; one band between 6000 bp and 5000 bp, and the other band between 4000 bp and 3000 bp. These bands correlate with the expected size of 5590 bp and 3424 bp, respectively (FIG. 42).

Table 33 below shows GATA3 specific TCR alpha and beta DNA sequence and codon optimized sequence.

| | Clonotype 1 TCR |
|---|---|
| Clonotype 1 TCR alpha | alpha codon optimized |
| ATGGCTTTTTGGCTGAGAAGGCTGGGTCTAC | ATGGCCTTCTGGCTGAGGAGACTGGGTTTAC |
| ATTTCAGGCCACATTTGGGGAGACGAATGGA | ACTTCAGACCCCATTTAGGCAGAAGAATGGA |
| GTCATTCCTGGGAGGTGTTTTGCTGATTTTG | GAGCTTTTTAGGCGGCGTGCTGCTGATTTTA |
| TGGCTTCAAGTGGACTGGGTGAAGAGCCAAA | TGGCTGCAAGTTGACTGGGTGAAGAGCCAGA |
| AGATAGAACAGAATTCCGAGGCCCTGAACAT | AGATCGAGCAGAACAGCGAGGCTTTAAACAT |
| TCAGGAGGGTAAAACGGCCACCCTGACCTGC | TCAAGAAGGCAAGACAGCCACTTTAACTTGT |
| AACTATACAAACTATTCTCCAGCATACTTAC | AACTATACCAACTACTCCCCCGCTTATTTAC |
| AGTGGTACCGACAAGATCCAGGAAGAGGCCC | AGTGGTACAGACAAGATCCCGGCAGAGGCCC |
| TGTTTTCTTGCTACTCATACGTGAAAATGAG | CGTGTTTTTACTGCTGATTCGTGAGAACGAG |
| AAAGAAAAAAGGAAAGAAAGACTGAAGGTCA | AAGGAGAAGAGGAAGGAGAGACTGAAGGTGA |
| CCTTTGATACCACCCTTAAACAGAGTTTGTT | CCTTCGACACCACTTTAAAGCAGTCTTTATT |
| TCATATCACAGCCTCCCAGCCTGCAGACTCA | CCACATCACCGCCAGCCAGCCCGCTGATAGC |
| GCTACCTACCTCTGTGCTCTAGACATTTATG | GCCACCTATTTATGCGCTTTAGACATCTACG |
| GGAACAACAGACTCGCTTTTGGGAAGGGGAA | GCACAAACAATCGTCTGGCCTTCGGCAAGGG |
| CGTGGTGGTCATACCA | CAACCAAGTTGTGGTGATCCCC |
| | Clonotype 1 TCR |
| Clonotype 1 TCR beta | beta codon optimized |
| ATGGGAATCAGGCTCCTCTGTCGTGTGGCCT | ATGGGCATTCGTCTGCTGTGTCGTGTGGCCT |
| TTTGTTTCCTGGCTGTAGGCCTCGTAGATGT | TCTGCTTTTTAGCCGTGGGTTTAGTGGACGT |
| GAAAGTAACCCAGAGCTCGAGATATCTAGTC | GAAGGTGACCCAGTCCTCTCGTTATTTAGTG |
| AAAAGGACGGGAGAGAAAGTTTTTCTGGAAT | AAGAGGACCGGCGAGAAGGTGTTTTTAGAAT |
| GTGTCCAGGATATGGACCATGAAAATATGTT | GCGTGCAAGATATGGACCACGAGAACATGTT |
| CTGGTATCGACAAGACCCAGGTCTGGGGCTA | CTGGTACAGACAAGATCCCGGACTGGGTTTA |
| CGGCTGATCTATTTCTCATATGATGTTAAAA | AGGCTGATCTACTTCAGCTACGACGTGAAGA |
| TGAAAGAAAAAGGAGATATTCCTGAGGGGTA | TGAAGGAGAAGGGCGACATCCCCGAGGGCTA |
| CAGTGTCTCTAGAGAGAAGAAGGAGCGCTTC | CTCCGTGTCTCGTGAGAAGAAGGAGAGGTTC |
| TCCCTGATTCTGGAGTCCGCCAGCACCAACC | TCTTTAATTTTAGAGTCCGCCAGCACCAACC |
| AGACATCTATGTACCTCTGTGCCAGCAGTTT | AGACCAGCATGTATTTATGCGCCAGCTCTTT |
| AGATTTTGTGCTAGCGGGGTCCTACTCCTAC | AGACTTTGTGCTGGCCGGCAGCTACAGCTAC |
| AATGAGCAGTTCTTCGGGCCAGGGACACGGC | AACGAGCAGTTCTTCGGCCCCGGCACCAGAC |
| TCACCGTGCTAG | TGACCGTGCTG |

### GATA3 Specific TCR Expression

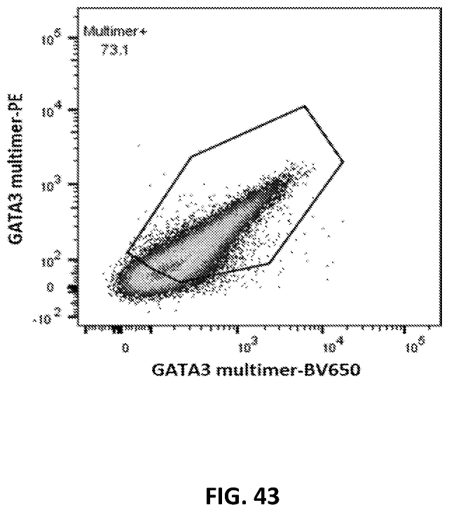

For GATA3 specific TCR expressed Jurkat cells, lenti-virus system was used after HEK 293T cell line transfection with GATA3 specific TCR construct and the lenti-virus was transduced into Jurkat cells. The transduced and puromycin selected Jurkat cells were stained with GATA3 multimer-PE and GATA3 multimer-BV650 and compared with non-transduced Jurkat cells to verify GATA3 specific TCR expression. 73.1% of cells were positive for both GATA3 multimer-PE and GATA3 multimer-BV650, indicating GATA3 neoORF specific TCR expression (FIG. 43)

### Peptide Titration Test

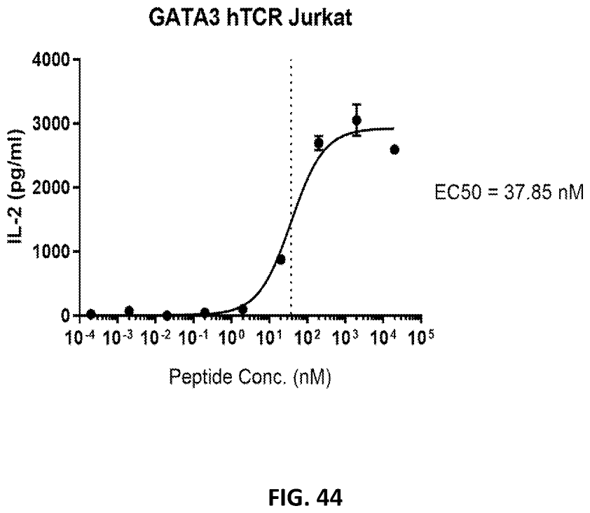

To verify the recombinant TCR is functional, peptide concentrations from 20 μM to 0.2 μM were tested with GATA3 specific TCR transduced Jurkat cells. IL-2 secretion levels from the Jurkat cells showed non-linear correlation with GATA3 peptide concentration with an observed EC₅₀=37.85 nM (FIG. 44).

### IL-2 Release Assay of GATA3 Specific TCR Transduced Jurkat

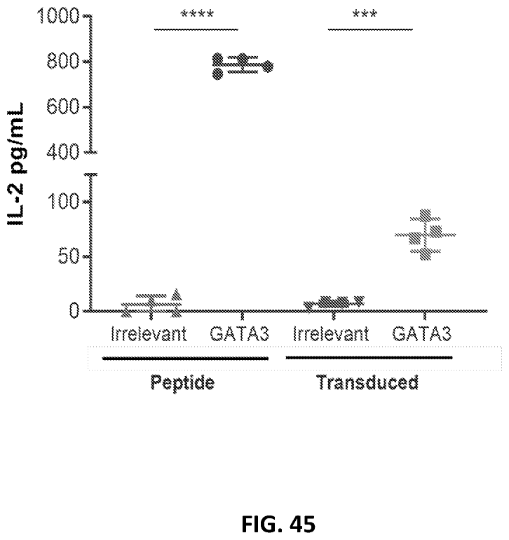

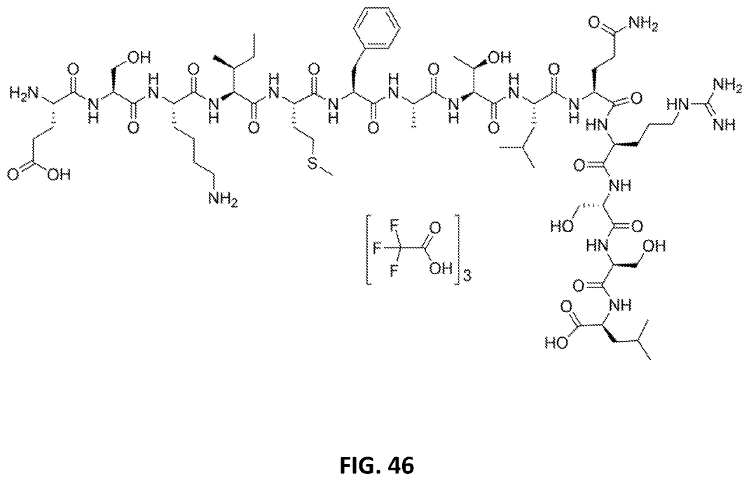

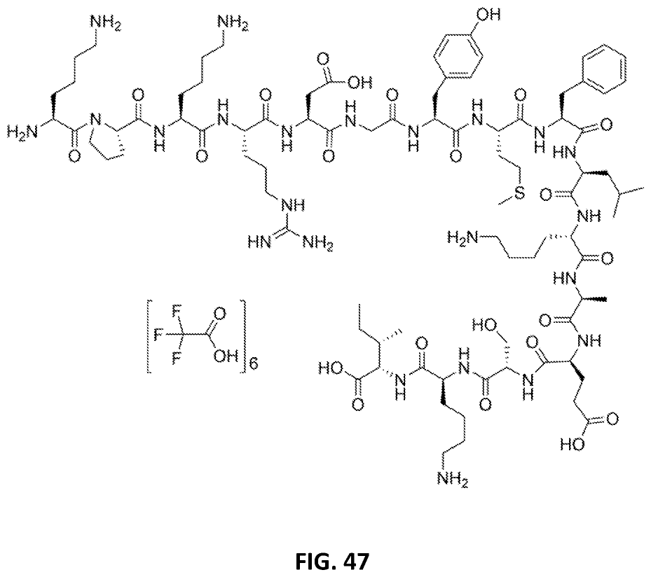

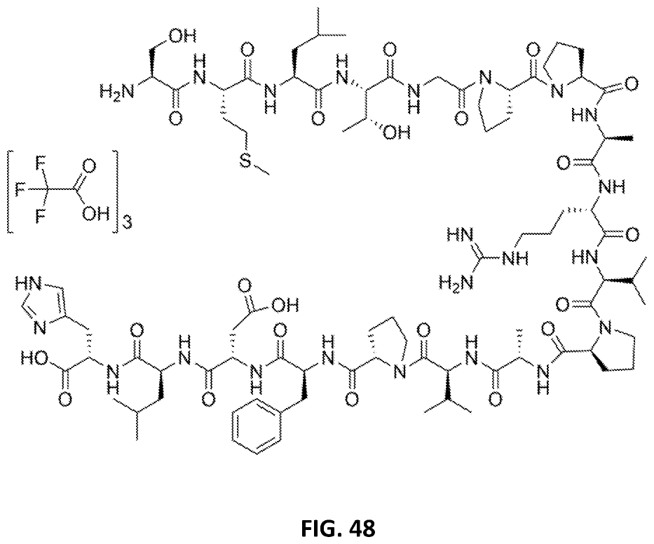

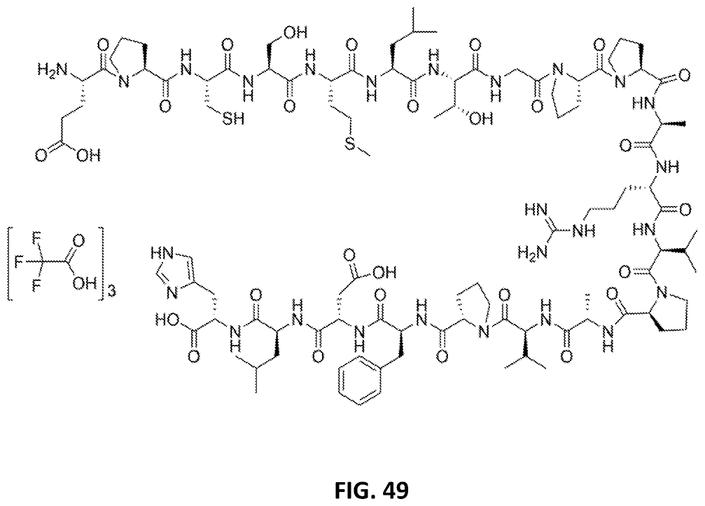

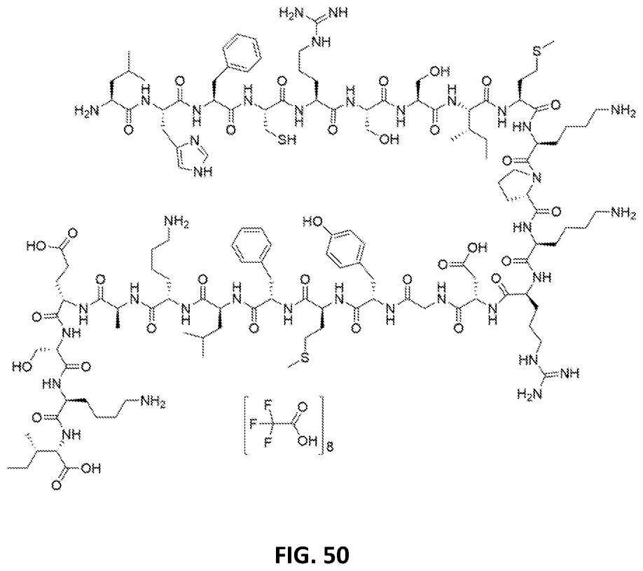

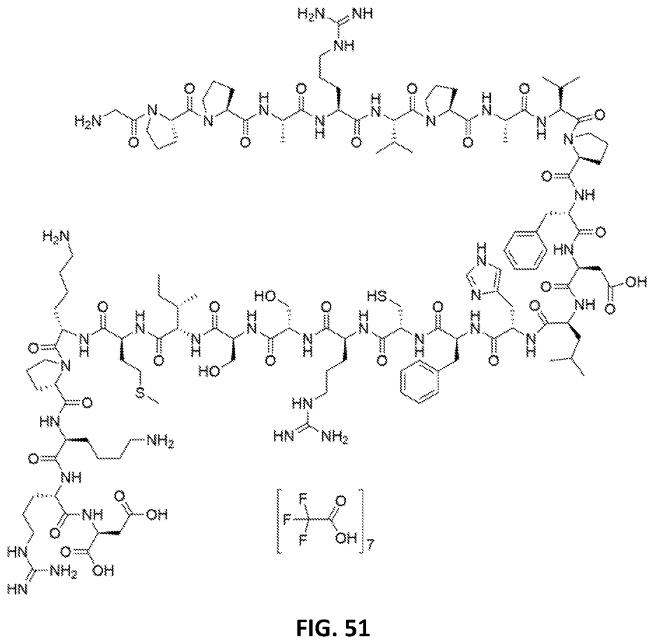

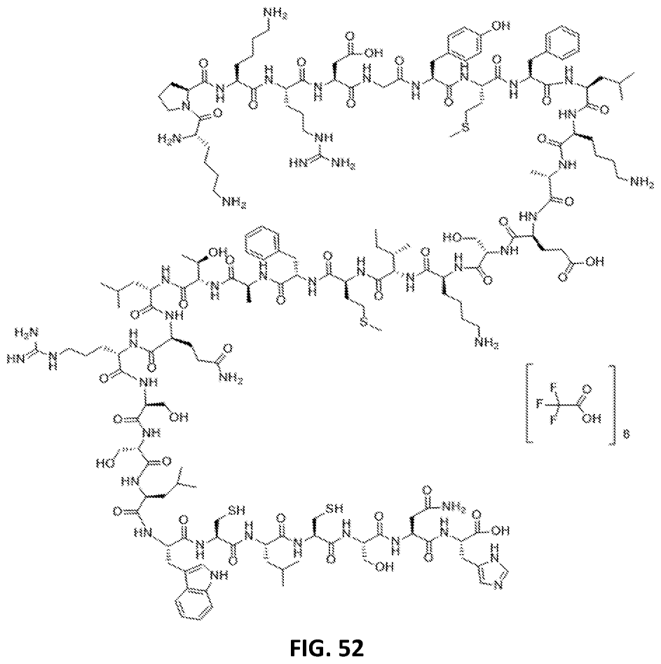

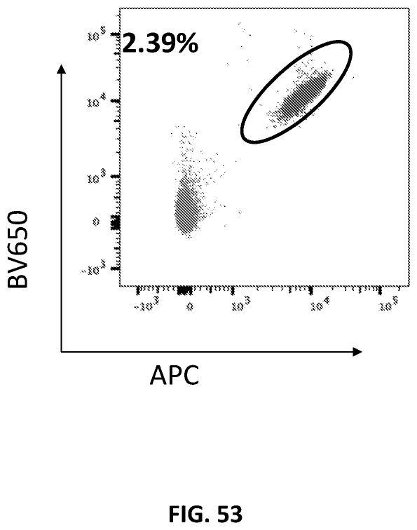

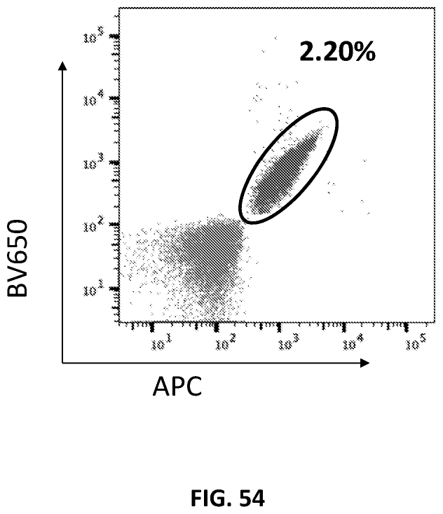

To verify the recognition of endogenous GATA3 mutation antigen by the GATA3 mutation specific TCR Jurkat, the mutation transduced HEK 293T cells were used as a target cell and co-cultured with GATA3 TCR transduced Jurkat cells. IL-2 level was higher in the GATA3 mutation peptide loaded HEK 293T cell group (circles) than irrelevant peptide loaded cell group (triangles) in FIG. 45. IL-2 level was also higher in the GATA3 mutation transduced target cell group (squares) than irrelevant gene transduced target cell group (inverted triangle) in FIG. 45.

In this study, a TCR was cloned from a CD8+ T cell specific for a GATA3 neoantigen presented on HLA-A02:01. The avidity of the TCR was defined to be less than 40 nM by peptide titration (EC50) and the TCR was able to recognize a GATA3 neoORF expressing cell line. These data confirm generation of CD8⁺ T cells that have potent TCRs that can recognize cells with the GATA3 neoORF mutation.

### Examples 27—Example 41 Described Below Relates to Mutant BTK and Mutant EGFR Peptides

### Example 27 Intracellular Cytokine Staining Assay

Induction of BTK neo-antigen specific CD4+ and CD8+ T cell responses and tetramer staining assay is performed as described in Example 1 and Example 2. Induction of EGFR neo-antigen specific CD4⁺ and CD8⁺ T cell responses and tetramer staining assay is performed as described in Example 1 and Example 2. In the absence of well-established tetramer staining to identify antigen-specific T cell populations, antigen-specificity can be estimated using assessment of cytokine production using well-established flow cytometry assays. Briefly, T cells are stimulated with the peptide of interest and compared to a control. After stimulation, production of cytokines by CD4⁺ T cells (e.g., IFNγ and TNFα) are assessed by intracellular staining. These cytokines, especially IFNγ, can be used to identify stimulated cells. FACS analysis of antigen-specific induction of IFNγ and TNFα levels of CD4+ cells from a healthy donor stimulated with APCs loaded with or without a mutant BTK is performed. FACS analysis of antigen-specific induction of IFNγ and TNFα levels of CD4+ cells from a healthy donor stimulated with APCs loaded with or without a mutant EGFR peptide is performed.

### Example 28—ELISPOT Assay

Peptide-specific T cells are functionally enumerated using the ELISPOT assay (BD Biosciences), which measures the release of IFNγ from T cells on a single cell basis. Target cells (T2 or HLA-A0201 transfected C1Rs) were pulsed with 10 μM peptide for 1 hour at 37° C., and washed three times. 1×10⁵ peptide-pulsed targets are co-cultured in the ELISPOT plate wells with varying concentrations of T cells (5×10² to 2×10³) taken from the immunogenicity culture. Plates are developed according to the manufacturer's protocol, and analyzed on an ELISPOT reader (Cellular Technology Ltd.) with accompanying software. Spots corresponding to the number of IFNγ-producing T cells are reported as the absolute number of spots per number of T cells plated. T cells expanded on modified peptides are tested not only for their ability to recognize targets pulsed with the modified peptide, but also for their ability to recognize targets pulsed with the parent peptide. The IFNγ levels of samples mock transduced or transduced with a lentiviral expression vector encoding a mutant BTK peptide or mutant EGFR peptide are determined.

### Example 29—CD107 Staining Assay

CD107a and b are expressed on the cell surface of CD8⁺ T cells following activation with cognate peptide. The lytic granules of T cells have a lipid bilayer that contains lysosomal-associated membrane glycoproteins (“LAMPs”), which include the molecules CD107a and b. When cytotoxic T cells are activated through the T cell receptor, the membranes of these lytic granules mobilize and fuse with the plasma membrane of the T cell. The granule contents are released, and this leads to the death of the target cell. As the granule membrane fuses with the plasma membrane, C107a and b are exposed on the cell surface, and therefore are markers of degranulation. Because degranulation as measured by CD107a and b staining is reported on a single cell basis, the assay is used to functionally enumerate peptide-specific T cells. To perform the assay, peptide is added to HLA-A02:01-transfected cells CIR to a final concentration of 20 μM, the cells are incubated for 1 hour at 37° C., and washed three times. 1×10⁵ of the peptide-pulsed C1R cells are aliquoted into tubes, and antibodies specific for CD107a and b are added to a final concentration suggested by the manufacturer (Becton Dickinson). Antibodies are added prior to the addition of T cells in order to “capture” the CD107 molecules as they transiently appear on the surface during the course of the assay. 1×10⁵ T cells from the immunogenicity culture are added next, and the samples were incubated for 4 hours at 37° C. The T cells are further stained for additional cell surface molecules such as CD8 and acquired on a FACS Calibur instrument (Becton Dickinson). Data is analyzed using the accompanying Cellquest software, and results are reported as the percentage of CD8⁺/CD107a and b⁺ cells.

### Example 30—Cytotoxicity Assays

Cytotoxic activity is measured using method 1 or method 2. Method 1 entails a chromium release assay. Target T2 cells are labeled for 1 hour at 37° C. with Na⁵¹Cr and washed 5×10³ target T2 cells are then added to varying numbers of T cells from the immunogenicity culture. Chromium release is measured in supernatant harvested after 4 hours of incubation at 37° C. The percentage of specific lysis is calculated as:

Experimental release−spontaneous release/Total release−spontaneous release×100. Equation 10.

In method 2 Cytotoxicity activity is measured with the detection of cleaved Caspase 3 in target cells by Flow cytometry. Target cancer cells are engineered to express the mutant peptide along with the proper MHC-I allele. Mock-transduced target cells (i.e. not expressing the mutant peptide) are used as a negative control. The cells are labeled with CFSE to distinguish them from the stimulated PBMCs used as effector cells. The target and effector cells are co-cultured for 6 hours before being harvested. Intracellular staining is performed to detect the cleaved form of Caspase 3 in the CFSE-positive target cancer cells. The percentage of specific lysis is calculated as:

Experimental cleavage of Caspase 3/spontaneous cleavage of Caspase 3(measured in the absence of mutant peptide expression)×100. Equation 11.

The method 2 cytotoxicity assay is provided in materials and methods section of Example 25 herein.

### Example 31—Enhanced CD8⁺ T Cell Responses In Vivo Using Longmers and Shortmers Sequentially

Vaccination with longmer peptides can induce both CD4⁺ and CD8⁺ T cell responses, depending on the processing and presentation of the peptides. Vaccination with minimal shortmer epitopes focuses on generating CD8⁺ T cell responses, but does not require peptide processing before antigen presentation. As such, any cell can present the epitope readily, not just professional antigen-presenting cells (APCs). This may lead to tolerance of T cells that come in contact with healthy cells presenting antigens as part of peripheral tolerance. To circumvent this, initial immunization with longmers allows priming of CD8⁺ T cells only by APCs that can process and present the peptides. Subsequent immunizations boosts the initial CD8⁺ T cell responses.

### In Vivo Immunogenicity Assays

Nineteen 8-12 week old female C57BL/6 mice (Taconic Biosciences) are randomly and prospectively assigned to treatment groups on arrival. Animals are acclimated for three (3) days prior to study commencement. Animals are maintained on LabDiet™ 5053 sterile rodent chow and sterile water provided ad libitum. Animals in Group 1 serve as vaccination adjuvant-only controls and are administered polyinosinic:polycytidylic acid (polyL:C) alone at 100 μg in a volume of 0.1 mL administered via subcutaneous injection (s.c.) on day 0, 7, and 14. Animals in Group 2 are administered 50 μg each of six longmer peptides (described below) along with polyL:C at 100 μg s.c. in a volume of 0.1 mL on day 0, 7 and 14. Animals in Group 3 are administered 50 μg each of six longmer peptides (described below) along with polyL:C at 100 μg s.c. in a volume of 0.1 mL on day 0 and molar-matched equivalents of corresponding shortmer peptides (described below) along with polyL:C at 100 μg s.c. in a volume of 0.1 mL on day 7 and 14. Animals are weighed and monitored for general health daily. Animals are euthanized by CO2 overdose at study completion Day 21, if an animal lost >30% of its body weight compared to weight at Day 0; or if an animal was found moribund. At sacrifice, spleens are harvested and processed into single-cell suspensions using standard protocols. Briefly, spleens are mechanical degraded through a 70 μM filter, pelleted, and lysed with ACK lysis buffer (Sigma) before resuspension in cell culture media.

### Peptides

Six previously identified murine neoantigens are used based on their demonstrated ability to induce CD8⁺ T cell responses. For each neoantigen, shortmers (8-11 amino acids) corresponding to the minimal epitope have been defined. Longmers corresponding to 20-27 amino acids surrounding the mutation are used.

### ELISPOT

ELISPOT analysis (Mouse IFNγ ELISPOT Reasy-SET-Go; EBioscience) is performed according to the kit protocol. Briefly, one day prior to day of analysis, 96-well filter plates (0.45 μm pore size hydrophobic PVDF membrane; EMD Millipore) are activated (35% EtOH), washed (PBS) and coated with capture antibody (1:250; 4° C. O/N). On the day of analysis, wells are washed and blocked (media; 2 hours at 37° C.). Approximately 2×10⁵ cells in 100 μL is added to the wells along with 100 μL of 10 mM test peptide pool (shortmers), or PMA/ionomycin positive control antigen, or vehicle. Cells are incubated with antigen overnight (16-18 hours) at 37° C. The next day, the cell suspension is discarded, and wells are washed once with PBS, and twice with deionized water. For all wash steps in the remainder of the assay, wells are allowed to soak for 3 minutes at each wash step. Wells are then washed three times with wash buffer (PBS+0.05% Tween-20), and detection antibody (1:250) is added to all wells. Plates are incubated for two hours at room temperature. The detection antibody solution is discarded, and wells are washed three times with wash buffer. Avidin-HRP (1:250) is added to all wells, and plates are incubated for one hour at room temperature. Conjugate solution is discarded, and wells washed three times with wash buffer, then once with PBS. Substrate (3-amino-9-ethyl-carbazole, 0.1 M Acetate buffer, H₂O₂) is added to all wells, and spot development monitored (approximately 10 minutes). Substrate reaction is stopped by washing wells with water, and plates are allowed to air-dry overnight. The plates are analyzed on an ELISPOT reader (Cellular Technology Ltd.) with accompanying software. Spots corresponding to the number of IFNγ-producing T cells are reported as the absolute number of spots per number of T cells plated.

### Example 32—Detection of Mutant BTK Peptides by Mass Spectrometry

293T cells are transduced with a lentiviral vector encoding various regions of a mutant BTK peptide. 50-100 million of the transduced cells expressing peptides encoded by the mutant BTK peptide are cultured and peptides are eluted from HLA-peptide complexes using an acid wash. Eluted peptides were then analyzed by MS/MS.

### Example 33 Mutant BTK Peptides Produce Strong Epitopes on Multiple Alleles

Multiple peptides containing the neoepitopes are expressed or loaded onto antigen presenting cells (APCs). Mass spectrometry was then performed and the affinity of the neoepitopes for the indicated HLA alleles and stability of the neoepitopes with the HLA alleles is determined.

### Example 34 Multiple BTK Neoepitopes Elicit CD8+ T Cell Responses

PBMC samples from a human donor can be used to perform antigen specific T cell induction. CD8+ T cell inductions are analyzed after manufacturing T cells. Cell samples can be taken out at different time points for analysis. pMHC multimers are used to monitor the fraction of antigen specific CD8+ T cells in the induction cultures.

### Example 35 Predicted HLA Specificities of Mutant BTK Neopeptides

Specific BTK neopeptides were run on proprietary RECON algorithm to predict HLA specificities. Neopeptides were ranked based on predicted binding affinities. Table 38 depicts the allelic specificities that are further ranked based on high to low affinity. A lower rank value indicates stronger affinity. Table 38 further demonstrates that the mutant BTK neopeptides identified and characterized herein have strong epitopes with multiple alleles.

| | IFIITEYMANGS | |
|---|---|---|
| BTK, C481S | LLNYLREMRHR | |
| PEPTIDE | ALLELE | RANK |
| ANGSLLNY | HLA-A36:01 | 24 |
| ANGSLLNYL | HLA-C15:02 | 14 |
| ANGSLLNYL | HLA-C08:01 | 19 |
| ANGSLLNYL | HLA-C06:02 | 19 |
| ANGSLLNYL | HLA-A02:04 | 21 |
| ANGSLLNYL | HLA-C12:02 | 25 |
| ANGSLLNYL | HLA-B44:02 | 26 |
| ANGSLLNYL | HLA-C17:01 | 27 |
| ANGSLLNYL | HLA-B38:01 | 27 |
| ANGSLLNYLR | HLA-A74:01 | 19 |
| ANGSLLNYLR | HLA-A31:01 | 26 |
| EYMANGSL | HLA-C14:02 | 13 |
| EYMANGSL | HLA-C14:03 | 13 |
| EYMANGSL | HLA-A24:02 | 25 |
| EYMANGSLL | HLA-A24:02 | 3 |
| EYMANGSLL | HLA-A23:01 | 9 |
| EYMANGSLL | HLA-C14:02 | 11 |
| EYMANGSLL | HLA-C14:03 | 12 |
| EYMANGSLL | HLA-A33:03 | 19 |
| EYMANGSLL | HLA-C04:01 | 20 |
| EYMANGSLL | HLA-B15:09 | 22 |
| EYMANGSLL | HLA-B38:01 | 23 |
| EYMANGSLLN | HLA-A24:02 | 24 |
| EYMANGSLLN | HLA-A23:01 | 27 |
| EYMANGSLLNY | HLA-A29:02 | 27 |
| GSLLNYLR | HLA-A31:01 | 16 |
| GSLLNYLR | HLA-A74:01 | 23 |
| GSLLNYLREM | HLA-B58:02 | 15 |
| GSLLNYLREM | HLA-B57:01 | 27 |
| ITEYMANGS | HLA-A01:01 | 23 |
| ITEYMANGSL | HLA-A01:01 | 20 |
| ITEYMANGSLL | HLA-A01:01 | 21 |
| MANGSLLNY | HLA-C02:02 | 1 |
| MANGSLLNY | HLA-C03:02 | 2 |
| MANGSLLNY | HLA-B53:01 | 2 |
| MANGSLLNY | HLA-B35:01 | 4 |
| MANGSLLNY | HLA-A29:02 | 11 |
| MANGSLLNY | HLA-C12:02 | 11 |
| MANGSLLNY | HLA-C12:03 | 11 |
| MANGSLLNY | HLA-A30:02 | 12 |
| MANGSLLNY | HLA-A36:01 | 12 |
| MANGSLLNY | HLA-A26:01 | 16 |
| MANGSLLNY | HLA-A01:01 | 17 |
| MANGSLLNY | HLA-B15:01 | 17 |
| MANGSLLNY | HLA-A25:01 | 18 |
| MANGSLLNY | HLA-B57:01 | 19 |
| MANGSLLNY | HLA-B58:01 | 22 |
| MANGSLLNY | HLA-A03:01 | 23 |
| MANGSLLNY | HLA-B46:01 | 23 |
| MANGSLLNY | HLA-B15:03 | 24 |
| MANGSLLNY | HLA-A33:03 | 25 |
| MANGSLLNY | HLA-B35:03 | 28 |
| MANGSLLNY | HLA-A11:01 | 28 |
| MANGSLLNYL | HLA-C17:01 | 17 |
| MANGSLLNYL | HLA-C02:02 | 18 |
| MANGSLLNYL | HLA-B35:01 | 18 |
| MANGSLLNYL | HLA-C03:03 | 21 |
| MANGSLLNYL | HLA-C08:01 | 24 |
| MANGSLLNYL | HLA-B35:03 | 24 |
| MANGSLLNYL | HLA-C12:02 | 25 |
| MANGSLLNYL | HLA-C01:02 | 26 |
| MANGSLLNYL | HLA-C03:04 | 28 |
| MANGSLLNYL | HLA-C08:02 | 28 |
| MANGSLLNYLR | HLA-A33:03 | 24 |
| MANGSLLNYLR | HLA-A74:01 | 28 |
| NGSLLNYL | HLA-B14:02 | 19 |
| NGSLLNYLR | HLA-A68:01 | 14 |
| NGSLLNYLR | HLA-A33:03 | 16 |
| NGSLLNYLR | HLA-A31:01 | 25 |
| NGSLLNYLR | HLA-A74:01 | 26 |
| SLLNYLREM | HLA-A02:04 | 5 |
| SLLNYLREM | HLA-A02:01 | 13 |
| SLLNYLREM | HLA-A02:03 | 16 |
| SLLNYLREM | HLA-C03:02 | 16 |
| SLLNYLREM | HLA-A03:01 | 19 |
| SLLNYLREM | HLA-A32:01 | 20 |
| SLLNYLREM | HLA-A02:07 | 20 |
| SLLNYLREM | HLA-C14:03 | 20 |
| SLLNYLREM | HLA-C14:02 | 20 |
| SLLNYLREM | HLA-A31:01 | 21 |
| SLLNYLREM | HLA-A30:02 | 22 |
| SLLNYLREM | HLA-A74:01 | 22 |
| SLLNYLREM | HLA-C06:02 | 24 |
| SLLNYLREM | HLA-B15:03 | 25 |
| SLLNYLREM | HLA-B46:01 | 25 |
| SLLNYLREM | HLA-B13:02 | 25 |
| SLLNYLREM | HLA-A25:01 | 26 |
| SLLNYLREM | HLA-A29:02 | 28 |
| SLLNYLREM | HLA-C01:02 | 28 |
| SLLNYLREMR | HLA-A74:01 | 14 |
| SLLNYLREMR | HLA-A31:01 | 20 |
| TEYMANGSL | HLA-B40:01 | 8 |
| TEYMANGSL | HLA-B40:02 | 8 |
| TEYMANGSL | HLA-B14:02 | 11 |
| TEYMANGSL | HLA-B49:01 | 14 |
| TEYMANGSL | HLA-B44:03 | 16 |
| TEYMANGSL | HLA-B44:02 | 17 |
| TEYMANGSL | HLA-B37:01 | 19 |
| TEYMANGSL | HLA-B18:01 | 20 |
| TEYMANGSL | HLA-B15:09 | 23 |
| TEYMANGSL | HLA-B41:01 | 25 |
| TEYMANGSL | HLA-B50:01 | 25 |
| TEYMANGSLL | HLA-B40:01 | 7 |
| TEYMANGSLL | HLA-B44:03 | 15 |
| TEYMANGSLL | HLA-B49:01 | 17 |
| TEYMANGSLL | HLA-B44:02 | 21 |
| TEYMANGSLL | HLA-B40:02 | 24 |
| TEYMANGSLLNY | HLA-B44:03 | 21 |
| YMANGSLL | HLA-B15:09 | 14 |
| YMANGSLL | HLA-C03:04 | 15 |
| YMANGSLL | HLA-C03:03 | 16 |
| YMANGSLL | HLA-C17:01 | 16 |
| YMANGSLL | HLA-C03:02 | 21 |
| YMANGSLL | HLA-C14:03 | 22 |
| YMANGSLL | HLA-C14:02 | 23 |
| YMANGSLL | HLA-C04:01 | 24 |
| YMANGSLL | HLA-C02:02 | 26 |
| YMANGSLL | HLA-A01:01 | 26 |
| YMANGSLLN | HLA-A29:02 | 25 |
| YMANGSLLN | HLA-A01:01 | 25 |
| YMANGSLLNY | HLA-A01:01 | 6 |
| YMANGSLLNY | HLA-A29:02 | 10 |
| YMANGSLLNY | HLA-A36:01 | 16 |
| YMANGSLLNY | HLA-A03:01 | 16 |
| YMANGSLLNY | HLA-B46:01 | 18 |
| YMANGSLLNY | HLA-A25:01 | 19 |
| YMANGSLLNY | HLA-B15:01 | 20 |
| YMANGSLLNY | HLA-A26:01 | 20 |
| YMANGSLLNY | HLA-A30:02 | 21 |
| YMANGSLLNY | HLA-A32:01 | 24 |

### Example 36 Affinity and Stability of Mutant BTK Neopeptides

Multiple peptides containing neoepitopes in the table below were either expressed or loaded onto antigen presenting cells. Mass spectrometry was then performed and the affinity of the neoepitopes for indicated HLA alleles were determined, and the stability of the neoepitopes with the HLA alleles were determined.

### Table 39 Shows the Respective Affinity and Stability of the Mutant BTK Peptides.

| | HLA | Peptide | Affinity | Stability |
|---|---|---|---|---|
| Gene | Allele | Sequence | (nM) | (1/2 hr) |
| BTK, C481S | A01.01 | YMANGSLLNY | 13.24495 | 0.866167 |
| BTK, C481S | A01.01 | MANGSLLNY | 439.029 | 0.216408 |
| BTK, C481S | A03.01 | MANGSLLNY | 35.62463 | 0.237963 |
| BTK, C481S | A03.01 | YMANGSLLNY | 95.93212 | 0.279088 |
| BTK, C481S | A11.01 | MANGSLLNY | 535.6333 | NB |
| BTK, C481S | A11.01 | YMANGSLLNY | 974.2881 | NB |
| BTK, C481S | A24.02 | EYMANGSLL | 4.961145 | 5.716141 |
| BTK_C481S | A02.01 | SLLNYLREM | 67.69132 | 3.043604 |
| BTK_C481S | A02.01 | MANGSLLNYL | 1006.566 | 0 |
| BTK_C481S | A02.01 | YMANGSLLN | 3999.442 | 0 |
| BTK_C481S | B07.02 | SLLNYLREM | 865.8805 | 0 |
| BTK_C481S | B07.02 | MANGSLLNYL | 16474.59 | 0 |
| BTK_C481S | B08.01 | SLLNYLREM | 959.6542 | 0 |
| BTK_C481S | B08.01 | MANGSLLNYL | 18463.09 | 0 |

### Example 37—Detection of Mutant EGFR Peptides by Mass Spectrometry

T cells are transduced with a lentiviral vector encoding various regions of a mutant EGFR peptide. 50-100 million of the transduced cells expressing peptides encoded by the mutant EGFR peptide are cultured and peptides are eluted from HLA-peptide complexes using an acid wash. Eluted peptides were then analyzed by MS/MS.

### Example 38—Mutant EGFR Peptides Produce Strong Epitopes on Multiple Alleles

Multiple peptides containing the neoepitopes are expressed or loaded onto antigen presenting cells (APCs). Mass spectrometry was then performed and the affinity of the neoepitopes for the indicated HLA alleles and stability of the neoepitopes with the HLA alleles is determined.

### Example 39—Multiple EGFR Neoepitopes Elicit CD8+ T Cell Responses

PBMC samples from a human donor can be used to perform antigen specific T cell induction. CD8⁺ T cell inductions are analyzed after manufacturing T cells. Cell samples can be taken out at different time points for analysis. pMHC multimers are used to monitor the fraction of antigen specific CD8⁺ T cells in the induction cultures.

### Example 40—Predicted HLA Specificities of EGFR Neopeptides

Specific neopeptides were run on proprietary RECON algorithm to predict HLA specificities. Neopeptides were ranked based on predicted binding affinities. Table 43 depicts the allelic specificities that are further ranked based on high to low affinity. A lower rank value indicates stronger affinity. Table 43 also demonstrates that the mutant EGFR neopeptides identified and characterized herein have strong epitopes with multiple alleles.

TABLE 43

| PEPTIDE | ALLELE | RANK |
|---|---|---|
| LIMQLMPF | HLA-C03:02 | 5 |
| LTSTVQLIM | HLA-C12:03 | 10 |
| | HLA-A01:01 | 13 |
| | HLA-C15:02 | 13 |
| | HLA-B57:01 | 14 |
| | HLA-B57:03 | 15 |
| | HLA-A36:01 | 16 |
| | HLA-C12:02 | 18 |
| | HLA-C03:03 | 19 |
| | HLA-B58:02 | 21 |
| QLIMQLMPF | HLA-B15:01 | 15 |
| | HLA-A26:01 | 21 |
| STVQLIMQL | HLA-A68:02 | 1 |
| | HLA-C15:02 | 2 |
| | HLA-A25:01 | 3 |
| | HLA-B57:03 | 4 |
| | HLA-C12:02 | 4 |
| | HLA-A26:01 | 5 |
| | HLA-C12:03 | 6 |
| | HLA-C06:02 | 7 |
| | HLA-C03:03 | 8 |
| | HLA-A30:01 | 9 |
| | HLA-C02:02 | 9 |
| | HLA-A11:01 | 10 |
| | HLA-A32:01 | 10 |
| | HLA-A02:04 | 10 |
| | HLA-A68:01 | 11 |
| | HLA-B15:09 | 11 |
| | HLA-C03:04 | 12 |
| | HLA-B38:01 | 18 |
| | HLA-B57:01 | 19 |
| | HLA-A02:03 | 20 |
| | HLA-C08:01 | 21 |
| | HLA-B35:01 | 21 |
| | HLA-B40:01 | 21 |
| STVQLIMQLM | HLA-A26:01 | 15 |
| | HLA-B57:01 | 17 |
| TSTVQLIMQL | HLA-C15:02 | 17 |
| TVQLIMQL | HLA-C17:01 | 11 |
| | HLA-B08:01 | 12 |
| | HLA-B42:01 | 13 |
| | HLA-B14:02 | 15 |
| | HLA-B37:01 | 15 |
| | HLA-B15:09 | 17 |
| TVQLIMQLM | HLA-B35:03 | 21 |
| VQLIMQLM | HLA-B52:01 | 8 |
| | HLA-B14:02 | 19 |
| | HLA-B37:01 | 19 |

### Example 41 Affinity and Stability of Mutant EGFR Neopeptides

Multiple peptides containing neoepitopes in the table below were either expressed or loaded onto antigen presenting cells. Mass spectrometry was then performed and the affinity of the neoepitopes for indicated HLA alleles were determined, and the stability of the neoepitopes with the HLA alleles were determined. Table 44 shows the respective affinity and stability of the mutant EGFR peptides.

TABLE 44

| | HLA | Peptide | Affinity | Stability |
|---|---|---|---|---|
| Gene | Allele | Sequence | (nM) | (1/2 hr) |
| EGFR, T790M | A01.01 | LTSTVQLIM | 2891.111 | 0.103721 |
| EGFR_T790M | A01.01 | CLTSTVQLIM | 8276.876 | 0 |
| EGFR_T790M | A02.01 | MQLMPFGCLL | 16.26147 | 0.381118 |
| EGFR_T790M | A02.01 | MQLMPFGCL | 116.3352 | 0.368273 |
| EGFR_T790M | A02.01 | LIMQLMPFGC | 132.4766 | 0.381284 |
| EGFR_T790M | A02.01 | QLIMQLMPF | 192.8406 | 0.34067 |
| EGFR_T790M | A02.01 | CLTSTVQLIM | 537.1391 | 0 |
| EGFR_T790M | A02.01 | IMQLMPFGCL | 653.1065 | 0.515559 |
| EGFR_T790M | A02.01 | IMQLMPFGC | 1205.368 | 0.370112 |
| EGFR_T790M | A02.01 | LIMQLMPFG | 3337.708 | 0 |
| EGFR_T790M | A02.01 | VQLIMQLMPF | 4942.892 | 0 |
| EGFR_T790M | A02.01 | QLIMQLMPFG | 5214.668 | 0 |
| EGFR_T790M | A02.01 | STVQLIMQL | 7256.773 | 0 |
| EGFR_T790M | A24.02 | QLIMQLMPF | 2030.807 | 0.368673 |
| EGFR_T790M | A24.02 | VQLIMQLMPF | 4103.131 | 0 |
| EGFR_T790M | A24.02 | IMQLMPFGCL | 14119.38 | 0 |
| EGFR_T790M | A24.02 | MQLMPFGCLL | 18857.47 | 0 |
| EGFR_T790M | B07.02 | MQLMPFGCL | 1589.188 | 0 |
| EGFR_T790M | B08.01 | QLIMQLMPF | 330.1933 | 0 |
| EGFR_T790M | B08.01 | IMQLMPFGCL | 427.3913 | 0 |
| EGFR_T790M | B08.01 | MQLMPFGCL | 4931.727 | 0 |
| EGFR_T790M | B08.01 | MQLMPFGCLL | 11244.9 | 0 |
| EGFR_T790M | B08.01 | VQLIMQLMPF | 16108.18 | 0 |
| EGFR_T790M | B08.02 | QLIMQLMPF | 5590.3 | ND |

What is claimed is:

1. A pharmaceutical composition comprising:
(a) at least one polypeptide or a pharmaceutically acceptable salt thereof comprising a first mutant GATA3 peptide sequence and a second mutant GATA3 peptide sequence, wherein
(i) the first mutant GATA3 peptide sequence and the second mutant GATA3 peptide sequence each comprise at least 8 contiguous amino acids of SEQ ID NO: 1, and
(ii) a C-terminal sequence of the first mutant GATA3 peptide sequence overlaps with an N-terminal sequence of the second mutant GATA3 peptide sequence; wherein the at least 8 contiguous amino acids of SEQ ID NO: 1 comprises at least one amino acid of sequence PGRPLQTHVL PEPHLALQPLQPHADHAHADAPAIQPVLWTTPPLQHGHRHGLEPCSMLTGPPARVPAVP FDLHFCRSSIMKPKRDGYMFLKAESKIMFAT LQRSSLWCLCSNH (SEQ ID NO: 2); or
(b) at least one polynucleotide comprising a sequence encoding the at least one polypeptide.

2. The pharmaceutical composition of claim 1, wherein the first mutant GATA3 peptide sequence or the second mutant GATA3 peptide sequence comprises at least 8 contiguous amino acids of SEQ ID NO: 2.

3. The pharmaceutical composition of any one of claims 1-2, wherein the first mutant GATA3 peptide sequence and the second mutant peptide sequence comprises at least 8 contiguous amino acids of SEQ ID NO: 2.

4. The pharmaceutical composition of any one of claims 2-3, wherein the at least 8 contiguous amino acids of SEQ ID NO: 2 comprises at least 8 contiguous amino acids of sequence PGRPLQTHVL PEPHLALQPLQPHADHAHADAPAIQPVLWTTPPLQHGHRHGL (SEQ ID NO: 3).

5. The pharmaceutical composition of any one of claims 2-4, wherein the at least 8 contiguous amino acids of SEQ ID NO: 2 comprises at least one amino acid of sequence EPCSMLTGPP ARVPAVPFDLHFCRSSIMKPKRDGYMFLKAESKIMFATLQRSSLWCLCSNH (SEQ ID NO: 4).

6. The pharmaceutical composition of any one of claims 1-5, wherein at least one of the first mutant GATA3 peptide sequence and the second mutant GATA3 peptide sequence comprise at least 14 mutant amino acids.

7. The pharmaceutical composition of any one of claims 1-6, wherein the at least one polypeptide comprises at least 3 mutant GATA3 peptide sequences.

8. The pharmaceutical composition of any one of claims 1-7, wherein the at least one polypeptide comprises at least two polypeptides.

9. The pharmaceutical composition of any one of claims 1-8, wherein the at least one polypeptide further comprises a third mutant GATA3 peptide sequence, wherein the third mutant GATA3 peptide sequence comprises at least 8 contiguous amino acids of SEQ ID NO: 1, wherein the at least 8 contiguous amino acids of SEQ ID NO: 1 comprises at least one amino acid of sequence SEQ ID NO: 2

10. The pharmaceutical composition of claim 9, wherein the third GATA3 mutant peptide comprises at least 8 contiguous amino acids of SEQ ID NO: 2.

11. The pharmaceutical composition of any one of claims 1-10, wherein the at least one polypeptide comprises at least one mutant GATA3 peptide sequence that binds to or is predicted to bind to a protein encoded by an HLA-A02:01 allele, an HLA-A24:02 allele, an HLA-A03:01 allele, an HLA-B07:02 allele and/or an HLA-B08:01 allele.

12. The pharmaceutical composition of any one of claims 1-11, wherein the at least one polypeptide comprises at least one mutant GATA3 peptide sequence that binds to or is predicted to bind to a protein encoded by:
(a) an HLA-A02:01 allele and an HLA-A24:02 allele;
(b) an HLA-A02:01 allele and an HLA-B08:01 allele;
(c) an HLA-A24:02 allele and an HLA-B08:01 allele; or
(d) HLA-A02:01 allele, an HLA-A24:02 allele and an HLA-B08:01 allele.

13. The pharmaceutical composition of any one of claims 1-12, wherein,
(a) the first mutant GATA3 peptide sequence binds to or is predicted to bind to a protein encoded by an HLA-A02:01 allele, an HLA-A24:02 allele, an HLA-A03:01 allele, an HLA-B07:02 allele or an HLA-B08:01 allele; and
(b) the second GATA3 peptide sequence binds to or is predicted to bind to a protein encoded by an HLA-A02:01 allele, an HLA-A24:02 allele, an HLA-A03:01 allele, an HLA-B07:02 allele or an HLA-B08:01 allele; wherein the first mutant GATA3 peptide sequence binds to or is predicted to bind to a protein encoded by different HLA allele than the second mutant GATA3 peptide sequence.

14. The pharmaceutical composition of any one of claims 1-13, wherein at least one of the first mutant GATA3 peptide sequence and the second mutant GATA 3 peptide sequence binds to a protein encoded by an HLA allele with an affinity of less than 500 nM.

15. The pharmaceutical composition of any one of claims 1-14, wherein at least one of the first mutant GATA3 peptide sequence and the second mutant peptide sequence binds to a protein encoded by an HLA allele with a stability of greater than 1 hour.

16. The pharmaceutical composition of any one of claims 1-15, wherein the at least one polypeptide comprises at least one of the following sequences:
(a) TLQRSSLWCL, VLPEPHLAL, HVLPEPHLAL, ALQPLQPHA, AIQPVLWTT, APAIQPVLWTT, SMLTGPPARV, MLTGPPARV, and/or YMFLKAESKI; and/or
(b) MFLKAESKI and/or YMFLKAESKI
(c) VLWTTPPLQH, YMFLKAESK and/or KIMFATLQR; and/or
(d) FATLQRSSL, EPHLALQPL, QPVLWTTPPL, GPPARVPAV, MFATLQRSSL KPKRDGYMF and/or KPKRDGYMFL and/or
(e) IMKPKRDGYM, MFATLQRSSL, FLKAESKIMF, LHFCRSSIM, EPHLALQPL, FATLQRSSL, ESKIMFATL, FLKAESKIM and/or YMFLKAESKI.

17. The pharmaceutical composition of any one of claims 1-16, wherein the at least one polypeptide comprises at least two of the following sequences:
(a) TLQRSSLWCL, VLPEPHLAL, HVLPEPHLAL, ALQPLQPHA, AIQPVLWTT, APAIQPVLWTT, SMLTGPPARV, MLTGPPARV, and/or YMFLKAESKI; and/or
(b) MFLKAESKI and/or YMFLKAESKI; and/or
(c) VLWTTPPLQH, YMFLKAESK and/or KIMFATLQR; and/or
(d) FATLQRSSL, EPHLALQPL, QPVLWTTPPL, GPPARVPAV, MFATLQRSSL KPKRDGYMF and/or KPKRDGYMFL and/or
(e) IMKPKRDGYM, MFATLQRSSL, FLKAESKIMF, LHFCRSSIM EPHLALQPL, FATLQRSSL, ESKIMFATL, FLKAESKIM and/or YMFLKAESKI.

18. The pharmaceutical composition of claim 16 or 17, wherein the mutant GATA3 peptide sequences comprise:
(a) the first mutant GATA3 peptide sequence from (a) and the second mutant GATA3 peptide sequence from (b);
(b) the first mutant GATA3 peptide sequence from (a) and the second mutant GATA3 peptide sequence from (c);
(c) the first mutant GATA3 peptide sequence from (a) and the second mutant GATA3 peptide sequence from (d);
(d) the first mutant GATA3 peptide sequence from (a) and the second mutant GATA3 peptide sequence from (e);
(e) the first mutant GATA3 peptide sequence from (b) and the second mutant GATA3 peptide sequence from (c);
(f) the first mutant GATA3 peptide sequence from (b) and the second mutant GATA3 peptide sequence from (d);
(g) the first mutant GATA3 peptide sequence from (b) and the second mutant GATA3 peptide sequence from (e);
(h) the first mutant GATA3 peptide sequence from (c) and the second mutant GATA3 peptide sequence from (d);
(i) the first mutant GATA3 peptide sequence from (c) and the second mutant GATA3 peptide sequence from (e); or
(j) the first mutant GATA3 peptide sequence from (d) and the second mutant GATA3 peptide sequence from (e).

19. The pharmaceutical composition of any one of claims 1-18, wherein the first mutant GATA3 peptide sequences, and the second mutant GATA 3 peptide sequence comprises a peptide of Table 5 and/or Table 6.

20. The pharmaceutical composition of any one of claims 1-19, wherein the first mutant GATA3 peptide sequence comprises a first neoepitope of GATA3 protein and the second peptide mutant GATA3 peptide sequence comprises a second neoepitope of a mutant GATA protein, wherein the first mutant GATA3 peptide sequence is different from the second mutant GATA3 peptide sequence, and wherein the first neoepitope comprises at least one mutant amino acid and the second neoepitope comprises the same mutant amino acid.

21. The pharmaceutical composition of any one of claims 1-20, wherein each of the first mutant GATA3 peptide sequence and the second mutant GATA3 peptide sequences comprising the at least eight contiguous amino acids are represented by a formula of
[Xaa]_{F}-[Xaa]_{N}-[Xaa]_{C} or [Xaa]_{N}-[Xaa]_{C}-[Xaa]_{F},
wherein each Xaa is an amino acid,
wherein [Xaa]_{N} and [Xaa]_{C} each comprise an amino acid sequence encoded by a different portion of the GATA3 gene,
wherein [Xaa]_{F} is any amino acid sequence,
wherein [Xaa]_{N} is encoded in a non-wild type reading frame of the GATA3 gene,
wherein [Xaa]_{C} comprises the at least one mutant amino acid and is encoded in a non-wild type reading frame of the GATA3 gene,
wherein N is an integer of from 0-100,
wherein C is an integer of from 1-100,
wherein F is an integer of from 0-100,
wherein the sum of N and M is at least 8.

22. The pharmaceutical composition of claim 21, wherein each Xaa of [Xaa]_{F} is a lysine residue and F is an integer of from 1-100, 1-10, 9, 8, 7, 6, 5, 4, 3, 2 or 1.

23. The pharmaceutical composition of claim 22, wherein F is 3, 4 or 5.

24. The pharmaceutical composition of any one of claims 1-23, wherein each of the mutant GATA3 peptide sequences are present at a concentration of at least 50 μg/mL-400 μg/mL.

25. The pharmaceutical composition of any one of claims 1-24, wherein the first mutant GATA3 peptide sequences and the second mutant GATA3 peptide sequence comprises a sequence of Table 1 or 2.

26. The pharmaceutical composition of any one of claims 1-25, wherein the composition further comprises an immunomodulatory agent or an adjuvant.

27. The pharmaceutical composition of claim 26, wherein the adjuvant is polyICLC.

28. A pharmaceutical composition comprising: one or more mutant GATA3 peptide sequence, the one or more mutant GATA3 peptide sequence comprises a sequence selected from group consisting of ESKIMFATLQRSSL, KPKRDGYMFLKAESKI, SMLTGPPARVPAVPFDLH, EPCSMLTGPPARVPAVPFDLH, LHFCRSSIMKPKRDGYMFLKAESKI, GPPARVPAVPFDLHFCRSSIMKPKRD, and KPKRDGYMFLKAESKIMFATLQRSSLWCLCSNH.

29. The pharmaceutical composition of any one of claims 1-28, wherein the pharmaceutical composition comprises a pH modifier present at a concentration of from 0.1 mM-1 mM.

30. The pharmaceutical composition of any one of claims 1-28, wherein the pharmaceutical composition comprises a pH modifier present at a concentration of from 1 mM-10 mM.

31. A method of synthesizing a GATA3 peptide, wherein the peptide comprises a sequence of at least two contiguous amino acids selected from the group consisting of Xaa-Cys, Xaa-Ser, and Xaa-Thr, wherein Xaa is any amino acid, the method comprising:
(a) coupling at least one di-peptide or derivative thereof to an amino acid or derivative thereof of a GATA3 peptide or derivative thereof to obtain a pseudo-proline containing GATA3 peptide or derivative thereof, wherein the di-peptide or derivative thereof comprises a pseudo-proline moiety;
(b) coupling one or more selected amino acids, small peptides or derivatives thereof to the pseudo-proline containing GATA3 peptide or derivative thereof; and
(c) cleaving the pseudo-proline containing GATA3 peptide or derivative thereof from the resin.

32. The method of claim 31, wherein the method comprises deprotecting the pseudo-proline containing GATA3 peptide or derivative thereof.

33. The method of any one of claims 31-32, wherein the amino acid or derivative thereof to which at least one di-peptide or derivative thereof is coupled is selected from the group consisting of Ala, Cys, Asp, Glu, Phe, Gly, Ile, Lys, Leu, Met, Asn, Pro, Gln, Arg, Ser, Thr, Trp, Tyr, His, and Val.

34. The method of any one of claims 31-33, wherein the one or more selected amino acids, small peptides or derivatives thereof optionally coupled to the pseudo-proline containing GATA3 peptide or derivative thereof comprise Fmoc-Ala-OH.H₂O, Fmoc-Cys(Trt)-OH, Fmoc-Asp(OtBu)-OH, Fmoc-Asp(OMpe)-OH, Fmoc-Glu(OtBu)-OH, Fmoc-Phe-OH, Fmoc-Gly-OH, Fmoc-Ile-OH, Fmoc-Lys(Boc)-OH, Fmoc-Leu-OH, Fmoc-Met-OH, Fmoc-Asn(Trt)-OH, Fmoc-Pro-OH, Fmoc-Gln(Trt)-OH, Fmoc-Arg(Pbf)-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-His(Trt)-OH and Fmoc-His(Boc)-OH.

35. The method of any one of claims 31-34, wherein an N-terminal amino acid or derivative thereof of the GATA3 peptide or derivative thereof is selected from the group consisting of Fmoc-Ala-OH.H₂O, Fmoc-Cys(Trt)-OH, Fmoc-Asp(OtBu)-OH, Fmoc-Asp(OMpe)-OH, Fmoc-Glu(OtBu)-OH, Fmoc-Phe-OH, Fmoc-Gly-OH, Fmoc-Ile-OH, Fmoc-Lys(Boc)-OH, Fmoc-Leu-OH, Fmoc-Met-OH, Fmoc-Asn(Trt)-OH, Fmoc-Pro-OH, Fmoc-Gln(Trt)-OH, Fmoc-Arg(Pbf)-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-His(Trt)-OH and Fmoc-His(Boc)-OH.

36. The method of any one of claims 31-35, wherein the pseudo-proline moiety is
(a) Fmoc-Ser(tBu)-Ser(psi(Me,Me)pro)-OH,
(b) Fmoc-Ala-Thr(psi(Me,Me)pro)-OH,
(c) Fmoc-Glu(OtBu)-Ser(psi(Me,Me)pro)-OH,
(d) Fmoc-Leu-Thr(psi(Me,Me)pro)-OH,
(e) Fmoc-Leu-Cys(psi(Dmp,H)pro)-OH.

37. The method of any one of claims 31-36, wherein
(a) Xaa-Ser is Ser-Ser,
(b) Xaa-Ser is Glu-Ser,
(c) Xaa-Thr is Ala-Thr,
(d) Xaa-Thr is Leu-Thr, or
(e) Xaa-Cys is Leu-Cys.

38. A method of treating a subject with cancer comprising administering to the subject the pharmaceutical composition of any one of claims 1-30.

39. A method of identifying a subject with cancer as a candidate for a therapeutic, the method comprising: identifying the subject as one that expresses a protein encoded by an HLA-A02:01 allele, an HLA-A24:02 allele, an HLA-A03:01 allele, an HLA-B07:02 allele and/or an HLA-B08:01 allele, wherein the therapeutic comprises
(a) at least one polypeptide comprising one or more mutant GATA3 peptide sequences, wherein each of the one or more mutant GATA3 peptide sequences comprises at least one mutant amino acid and is fragment of at least 8 contiguous amino acids of a mutant GATA3 protein arising from a mutation in a GATA3 gene of a cancer cell; or
(b) at least one polynucleotide comprising a sequence encoding the at least one polypeptide, wherein each of the one or more mutant GATA3 peptide sequences or a portion thereof binds to a protein encoded by an HLA-A02:01 allele, an HLA-A24:02 allele, an HLA-A03:01 allele, an HLA-B07:02 allele and/or an HLA-B08:01 allele.

40. The method of claim 39, wherein the method further comprises administering the therapeutic to the subject.

41. A method of treating a subject with cancer comprising administering to the subject a pharmaceutical composition comprising:
(a) at least one polypeptide comprising a first mutant GATA3 peptide sequence and a second mutant GATA3 peptide sequence, wherein
(i) the first mutant GATA3 peptide sequence and the second mutant GATA3 peptide sequence each comprise at least 8 contiguous amino acids of SEQ ID NO: 1, and
(ii) a C-terminal sequence of the first mutant GATA3 peptide sequence overlaps with an N-terminal sequence of the second mutant GATA3 peptide sequence; wherein the at least 8 contiguous amino acids of SEQ ID NO: 1 comprises at least one amino acid of sequence PGRPLQTHVL PEPHLALQPLQPHADHAHADAPAIQPVLWTTPPLQHGHRHGLEPCSMLTGPPARVPAVP FDLHFCRSSIMKPKRDGYMFLKAESKIMFAT LQRSSLWCLCSNH (SEQ ID NO: 2); or
(b) at least one polynucleotide comprising a sequence encoding the at least one polypeptide, wherein HLA alleles expressed by subject are unknown at the time of administering.

42. The method of claim 41, wherein the at least 8 contiguous amino acid of SEQ ID NO: 1 comprises at least one amino acid of sequence: PGRPLQTHVLPEPHLALQPLQPHADHAHADAPAIQPVLWTTPPLQHGHRHGLEPCSMLTGPPAR VPAVPFDLHFCRSSIMKPKRDGYMFLKAESKIMFAT LQRSSLWCLCSNH (SEQ ID NO: 2).

43. The method of any one of claims 41-42, wherein the cancer is selected from the group consisting of melanoma, ovarian cancer, lung cancer, prostate cancer, breast cancer, colorectal cancer, endometrial cancer, and chronic lymphocytic leukemia (CLL).

44. The method of any one of claims 41-43, wherein the subject has a breast cancer that is resistant to anti-estrogen therapy, is an MSI breast cancer, is a metastatic breast cancer, is a Her2 negative breast cancer, is a Her2 positive breast cancer, is an ER negative breast cancer, is an ER positive breast cancer, PR positive breast cancer, PR negetive breast cancer or any combination thereof.

45. The method of claim 44, wherein the breast cancer expresses an estrogen receptor with a mutation.

46. The method of any one of claims 41-45, further comprising administering at least one additional therapeutic agent or modality.

47. The method of claim 46, wherein the at least one additional therapeutic agent or modality is surgery, a checkpoint inhibitor, an antibody or fragment thereof, a chemotherapeutic agent, radiation, a vaccine, a small molecule, a T cell, a vector, and APC, a polynucleotide, an oncolytic virus or any combination thereof.

48. The method of claim 47, wherein the at least one additional therapeutic agent is an anti-PD-1 agent and anti-PD-L1 agent, an anti-CTLA-4 agent, an anti-CD40 agent, letrozole, fulvestrant, a PI3 kinase inhibitor and/or a CDK 4/6 inhibitor.

49. The method of claim 47, wherein the at least one additional therapeutic agent is palbociclib, ribociclib, abemaciclib, seliciclib, dinaciclib, milciclib, roniciclib, atuveciclib, briciclib, riviciclib, seliciclib, trilaciclib, voruciclib or any combination thereof.

50. The method of claim 47, wherein the at least one additional therapeutic agent is palbociclib (PD0332991); abemaciclib (LY2835219); ribociclib (LEE 011); voruciclib (P1446A-05); fascaplysin; arcyriaflavin; 2-bromo-12,13-dihydro-5H-indolo[2,3-a]pyrrolo[3,4-c]carbazole-5,7(6H)-dione; 3-amino thioacridone (3-ATA), trans-4-((6-(ethylamino)-2-((1-(phenylmethyl)-1H-indol-5-yl)amino)-4-pyrimidinyl)amino)-cyclohexano (CINK4); 1,4-dimethoxyacridine-9(10H)-thione (NSC 625987); 2-methyl-5-(p-tolylamino)benzo[d]thiazole-4,7-dione (ryuvidine); flavopiridol (alvocidib); seliciclib; dinaciclib; milciclib; roniciclib; atuveciclib; briciclib; riviciclib; trilaciclib (G1T28); or any combination thereof.

51. The method of claim 47, wherein the at least one additional therapeutic agent is Wortmannin, Demethoxyviridin, LY294002, hibiscone C, Idelalisib, Copanlisib, Duvelisib, Taselisib, Perifosine, Buparlisib, Duvelisib, Alpelisib (BYL719), Umbralisib, (TGR 1202), Copanlisib (BAY 80-6946), PX-866, Dactolisib, CUDC-907, Voxtalisib (SAR245409, XL765), CUDC-907, ME-401, IPI-549, SF1126, RP6530, INK1117, pictilisib (GDC-0941), XL147 (SAR245408), Palomid 529, GSK1059615, ZSTK474, PWT33597, IC87114, TG100-115, CAL263, RP6503, PI-103, GNE-477 or AEZS-136.

52. The method of any one of claims 41-51, wherein the cancer is recurrent or metastatic breast cancer.

53. The method of any one of claims 41-52, wherein the subject is a subject that has had disease progression following endocrine therapy in combination with a CDK 4/6 inhibitor; or wherein the subject has not received prior systemic therapy.

54. The method of any one of claims 41-53, wherein the method comprises determining a mutation status of an estrogen receptor gene of cells of the subject.

55. The method of claim 54, wherein the cells are isolated cells or cells enriched for expression of estrogen receptor.
